(19) United States
(12) Patent Application Publication
(10) Pub. No.: US 2023/0047519 A1
(43) Pub. Date: Feb. 16, 2023

(54) ORGANIC ELECTROLUMINESCENT MATERIAL AND DEVICE THEREOF

(71) Applicant: BEIJING SUMMER SPROUT TECHNOLOGY CO., LTD., Beijing (CN)

(72) Inventors: Zheng Wang, Beijing (CN); Hongbo Li, Beijing (CN); Ming Sang, Beijing (CN); Zhen Wang, Beijing (CN); Wei Cai, Beijing (CN); Chi Yuen Raymond Kwong, Beijing (CN); Chuanjun Xia, Beijing (CN)

(73) Assignee: BEIJING SUMMER SPROUT TECHNOLOGY CO., LTD., Beijing (CN)

(21) Appl. No.: 17/856,067

(22) Filed: Jul. 1, 2022

(30) Foreign Application Priority Data
Jul. 2, 2021 (CN) 202110747323.7
Jun. 22, 2022 (CN) 202210612368.8

(51) Int. Cl. H01L 51/00; H01L 51/52; C07D 487/04; C07D 487/14

(52) U.S. Cl. CPC H01L 51/0084; H01L 51/0054; H01L 51/0067; H01L 51/0072; H01L 51/0073; H01L 51/0074; H01L 51/0094; H01L 51/5206; H01L 51/5221; C07D 487/04; C07D 487/14

(57) ABSTRACT

Provided are an organic electroluminescent material and a device comprising the same. The organic electroluminescent material is a metal complex comprising a ligand Lₐ having a structure of Formula 1A and a ligand L_{b} having a structure of Formula 1B. Such new compounds each have a lower evaporation temperature, which is conducive to industrial application of the material and can reduce energy consumption in industrialization. Such metal complexes are used as a light-emitting material in an electroluminescent device. When applied to the electroluminescent device, such metal complexes can provide very excellent device performance, especially an improved device lifetime. Further provided are an organic electroluminescent device comprising the metal complex and a compound composition comprising the metal complex.

### CROSS-REFERENCE TO RELATED APPLICATION(S)

This application claims priority to Chinese Patent Application No. 202110747323.7 filed on Jul. 2, 2021 and Chinese Patent Application No. 202210612368.8 filed on Jun. 2, 2022, the disclosure of which are incorporated herein by reference in their entireties.

### TECHNICAL FIELD

The present disclosure relates to compounds for organic electronic devices such as organic light-emitting devices. In particular, the present disclosure relates to a metal complex comprising a ligand Lₐ having a structure of Formula 1A and a ligand L_{b} having a structure of Formula 1B and an organic electroluminescent device and compound composition comprising the metal complex.

### BACKGROUND

Organic electronic devices include, but are not limited to, the following types: organic light-emitting diodes (OLEDs), organic field-effect transistors (O-FETs), organic light-emitting transistors (OLETs), organic photovoltaic devices (OPVs), dye-sensitized solar cells (DSSCs), organic optical detectors, organic photoreceptors, organic field-quench devices (OFQDs), light-emitting electrochemical cells (LECs), organic laser diodes and organic plasmon emitting devices.

In 1987, Tang and Van Slyke of Eastman Kodak reported a bilayer organic electroluminescent device, which includes an arylamine hole transporting layer and a tris-8-hydroxyquinolato-aluminum layer as the electron and emitting layer (Applied Physics Letters, 1987, 51 (12): 913-915). Once a bias is applied to the device, green light was emitted from the device. This device laid the foundation for the development of modern organic light-emitting diodes (OLEDs). State-of-the-art OLEDs may include multiple layers such as charge injection and transporting layers, charge and exciton blocking layers, and one or multiple emissive layers between the cathode and anode. Since the OLED is a self-emitting solid state device, it offers tremendous potential for display and lighting applications. In addition, the inherent properties of organic materials, such as their flexibility, may make them well suited for particular applications such as fabrication on flexible substrates.

The OLED can be categorized as three different types according to its emitting mechanism. The OLED invented by Tang and van Slyke is a fluorescent OLED. It only utilizes singlet emission. The triplets generated in the device are wasted through nonradiative decay channels. Therefore, the internal quantum efficiency (IQE) of the fluorescent OLED is only 25%. This limitation hindered the commercialization of OLED. In 1997, Forrest and Thompson reported phosphorescent OLED, which uses triplet emission from heavy metal containing complexes as the emitter. As a result, both singlet and triplets can be harvested, achieving 100% IQE. The discovery and development of phosphorescent OLED contributed directly to the commercialization of active-matrix OLED (AMOLED) due to its high efficiency. Recently, Adachi achieved high efficiency through thermally activated delayed fluorescence (TADF) of organic compounds. These emitters have small singlet-triplet gap that makes the transition from triplet back to singlet possible. In the TADF device, the triplet excitons can go through reverse intersystem crossing to generate singlet excitons, resulting in high IQE.

OLEDs can also be classified as small molecule and polymer OLEDs according to the forms of the materials used. A small molecule refers to any organic or organometallic material that is not a polymer. The molecular weight of the small molecule can be large as long as it has well defined structure. Dendrimers with well-defined structures are considered as small molecules. Polymer OLEDs include conjugated polymers and non-conjugated polymers with pendant emitting groups. Small molecule OLED can become the polymer OLED if post polymerization occurred during the fabrication process.

There are various methods for OLED fabrication. Small molecule OLEDs are generally fabricated by vacuum thermal evaporation. Polymer OLEDs are fabricated by solution process such as spin-coating, inkjet printing, and slit printing. If the material can be dissolved or dispersed in a solvent, the small molecule OLED can also be produced by solution process.

The emitting color of the OLED can be achieved by emitter structural design. An OLED may include one emitting layer or a plurality of emitting layers to achieve desired spectrum. In the case of green, yellow, and red OLEDs, phosphorescent emitters have successfully reached commercialization. Blue phosphorescent device still suffers from non-saturated blue color, short device lifetime, and high operating voltage. Commercial full-color OLED displays normally adopt a hybrid strategy, using fluorescent blue and phosphorescent yellow, or red and green. At present, efficiency roll-off of phosphorescent OLEDs at high brightness remains a problem. In addition, it is desirable to have more saturated emitting color, higher efficiency, and longer device lifetime.

US20190280221A1 has disclosed a metal complex comprising a ligand having the following structure:

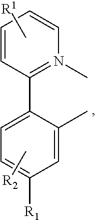

and the metal complex further has the following structure of a general formula:

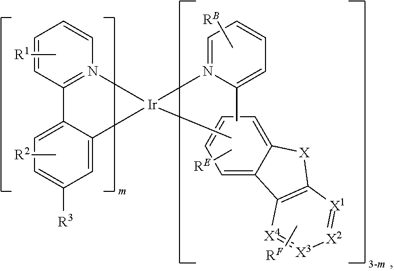

wherein R³ is selected from alkyl or cycloalkyl, and R¹ represents mono-substitution, multiple substitutions or non-substitution. The application has further disclosed the following specific structures:

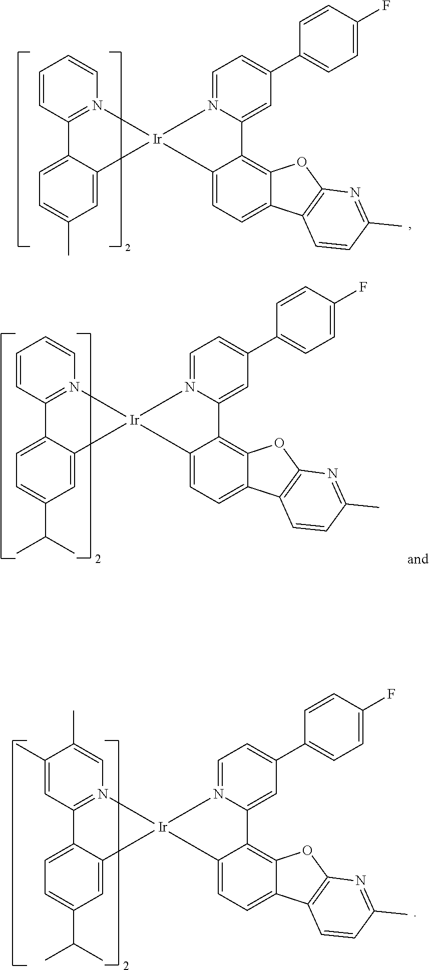

However, the application has not disclosed or taught a metal complex where R¹ and R³ are particular substitutions at the same time and an effect of the metal complex on performance of an organic electroluminescent device.

EP3663308A1 has disclosed a metal complex comprising ligands having the following structures:

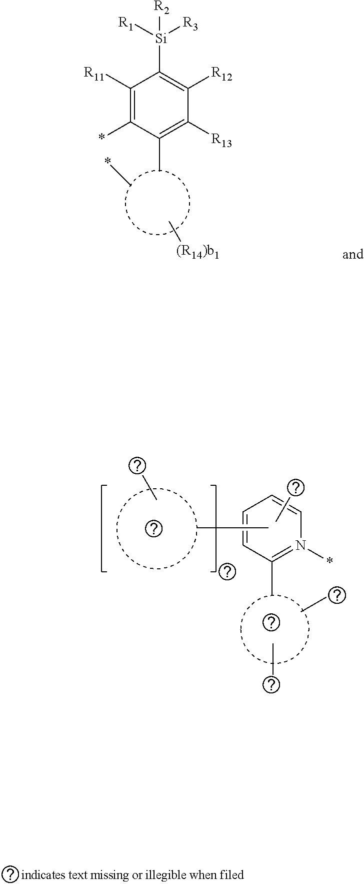

and the metal complex further has the following structure of a general formula:

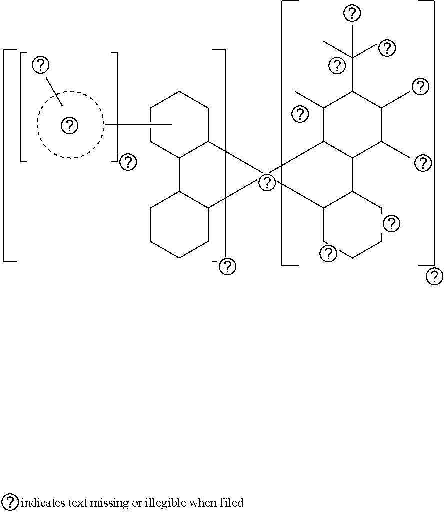

wherein R₁₂ is neither hydrogen nor methyl, CY₁ is selected from a C₅-C₃₀ carboncyclic ring or a C₁-C₃₀ heterocyclic ring, and c1 is selected from 1 to 4. The application has further disclosed the following specific structure:

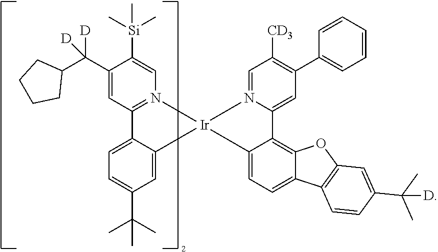

The application has disclosed a metal complex where pyridyl in one ligand comprises a silyl substitution and a substituent which is neither hydrogen nor methyl and pyridyl in another ligand has a substituent which is a carboncyclic ring or a heterocyclic ring and an effect of the metal complex on performance of an organic electroluminescent device. However, the application has not disclosed and taught a metal complex where both Z₁ and Z₂ are particular substitutions and an effect of the metal complex on the device performance.

### SUMMARY

The present disclosure aims to provide a series of metal complexes each comprising a ligand Lₐ having a structure of Formula 1A and a ligand L_{b} having a structure of Formula 1B to solve at least part of the preceding problems. These metal complexes may be used as a light-emitting material in an electroluminescent device. Such novel metal complexes each have a lower evaporation temperature. When applied to the electroluminescent device, such novel metal complexes can provide better device performance such as an improved device lifetime and a narrower full width at half maximum (FWHM).

According to an embodiment of the present disclosure, disclosed is a metal complex having a general formula of M(Lₐ)ₘ(L_{b})ₙ(L_{c})_{q};

wherein

Lₐ, L_{b} and L_{c} are a first ligand, a second ligand and a third ligand coordinated to the metal M, respectively, and Lₐ, L_{b} and L_{c} are the same or different; wherein Lₐ, L_{b} and L_{c} can be optionally joined to form a multidentate ligand;

the metal M is selected from a metal with a relative atomic mass greater than 40; and

m is selected from 1 or 2, n is selected from 1 or 2, q is selected from 0 or 1, and m+n+q equals an oxidation state of M; when m is 2, two Lₐ are identical or different; when n is 2, two L_{b} are identical or different;

Lₐ has, at each occurrence identically or differently, a structure represented by Formula 1A and L_{b} has, at each occurrence identically or differently, a structure represented by Formula 1B:

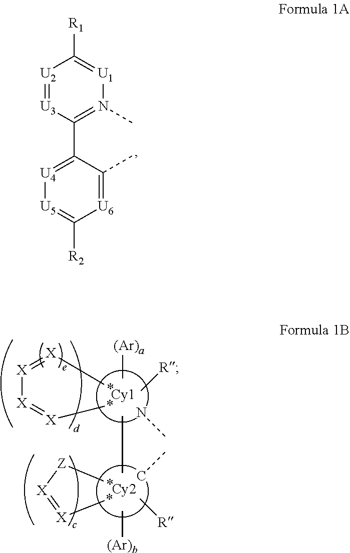

wherein

the ring Cy1 is, at each occurrence identically or differently, selected from a heteroaromatic ring having 5 to 6 ring atoms;

the ring Cy2 is, at each occurrence identically or differently, selected from a benzene ring or a heteroaromatic ring having 5 to 6 ring atoms;

Ar is, at each occurrence identically or differently, selected from substituted or unsubstituted aryl having 6 to 30 carbon atoms, substituted or unsubstituted heteroaryl having 3 to 30 carbon atoms or a combination thereof;

a, b, c, d and e are, at each occurrence identically or differently, selected from 0 or 1, and at least one of a, b, c and d is selected from 1;

Z is, at each occurrence identically or differently, selected from O or X═X;

X is, at each occurrence identically or differently, selected from N or CR′;

U₁ to U₆ are, at each occurrence identically or differently, selected from CRᵤ or N;

R″ is, at each occurrence identically or differently, selected from mono-substitution, multiple substitutions or non-substitution;

R′, R″ and Rᵤ are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group and combinations thereof;

“*” represents a position where the ring Cy1 or the ring Cy2 is joined;

in Formula 1A, adjacent substituents Rᵤ can be optionally joined to form a ring;

in Formula 1B, adjacent substituents R′ and R″ can be optionally joined to form a ring; and

R₁ is, at each occurrence identically or differently, selected from the group consisting of: substituted or unsubstituted alkyl having 4 to 20 carbon atoms, substituted or unsubstituted cycloalkyl having 4 to 20 ring carbon atoms, substituted or unsubstituted heteroalkyl having 4 to 20 carbon atoms, a substituted or unsubstituted heterocyclic group having 4 to 20 ring atoms, substituted or unsubstituted arylalkyl having 7 to 30 carbon atoms, substituted or unsubstituted alkoxy having 4 to 20 carbon atoms, substituted or unsubstituted aryloxy having 6 to 30 carbon atoms, substituted or unsubstituted alkenyl having 4 to 20 carbon atoms, substituted or unsubstituted alkynyl having 4 to 20 carbon atoms, substituted or unsubstituted aryl having 6 to 30 carbon atoms, substituted or unsubstituted heteroaryl having 4 to 30 carbon atoms and combinations thereof;

R₂ has a structure represented by Formula 2:

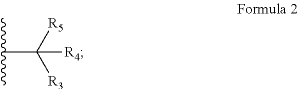

wherein

the number of carbon atoms in R₂ is greater than or equal to 4;

R₃, R₄ and R₅ are, at each occurrence identically or differently, selected from the group consisting of: halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group and combinations thereof;

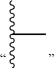

represents a position where Formula 2 is joined to Formula 1A;

two adjacent substituents of R₃, R₄ and R₅ can be optionally joined to form a ring; and

L_{c} is a monoanionic bidentate ligand.

According to another embodiment of the present disclosure, further disclosed is an electroluminescent device. The electroluminescent device includes:

an anode,

a cathode, and

an organic layer disposed between the anode and the cathode, wherein at least one layer of the organic layer comprises the metal complex according to the preceding embodiment.

According to another embodiment of the present disclosure, further disclosed is a compound composition. The compound composition comprises the metal complex according to the preceding embodiment.

The present disclosure has disclosed the series of metal complexes each comprising the ligand Lₐ having the structure of Formula 1A and the ligand L_{b} having the structure of Formula 1B, and such novel metal complexes each have the lower evaporation temperature. Since a large number of materials need to be heated and evaporated for a long time in an industrial process, the low evaporation temperature not only can reduce energy consumption in industrialization, but is also conducive to improving thermal stability of the material in a process of preparing the device and conducive to industrial application of the material. Such metal complexes may be used as the light-emitting material in the electroluminescent device. When such metal complexes are applied to the electroluminescent device, the electroluminescent device can obtain very excellent device performance, especially an improved device lifetime which is difficult to predict.

### BRIEF DESCRIPTION OF DRAWINGS

FIG. 1 is a schematic diagram of an organic light-emitting apparatus that may comprise a metal complex and a compound composition disclosed herein.

FIG. 2 is a schematic diagram of another organic light-emitting apparatus that may comprise a metal complex and a compound composition disclosed herein.

### DETAILED DESCRIPTION

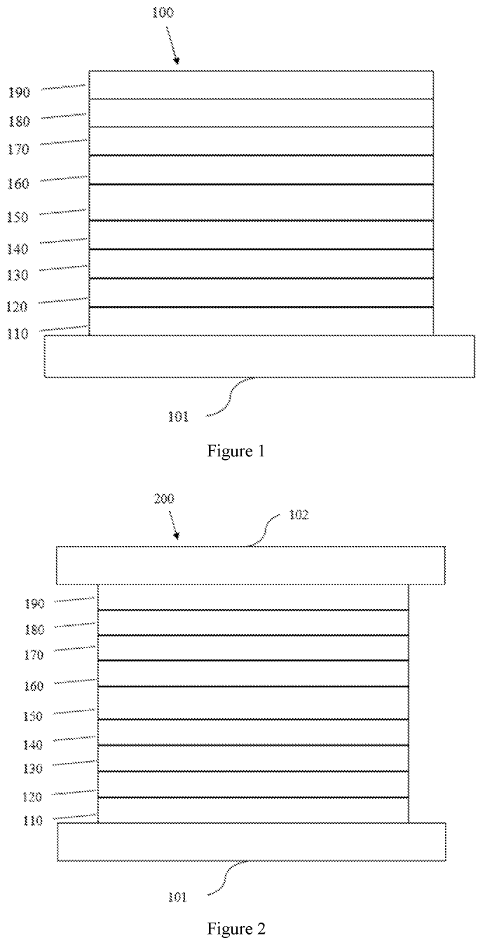

OLEDs can be fabricated on various types of substrates such as glass, plastic, and metal foil. FIG. 1 schematically shows an organic light-emitting device 100 without limitation.

The figures are not necessarily drawn to scale. Some of the layers in the figures can also be omitted as needed. Device 100 may include a substrate 101, an anode 110, a hole injection layer 120, a hole transport layer 130, an electron blocking layer 140, an emissive layer 150, a hole blocking layer 160, an electron transport layer 170, an electron injection layer 180 and a cathode 190. Device 100 may be fabricated by depositing the layers described in order. The properties and functions of these various layers, as well as example materials, are described in more detail in U.S. Pat. No. 7,279,704 at cols. 6-10, the contents of which are incorporated by reference herein in its entirety.

More examples for each of these layers are available. For example, a flexible and transparent substrate-anode combination is disclosed in U.S. Pat. No. 5,844,363, which is incorporated by reference herein in its entirety. An example of a p-doped hole transport layer is m-MTDATA doped with F4-TCNQ at a molar ratio of 50:1, as disclosed in U.S. Patent Application Publication No. 2003/0230980, which is incorporated by reference herein in its entirety. Examples of host materials are disclosed in U.S. Pat. No. 6,303,238 to Thompson et al., which is incorporated by reference herein in its entirety. An example of an n-doped electron transport layer is BPhen doped with Li at a molar ratio of 1:1, as disclosed in U.S. Patent Application Publication No. 2003/0230980, which is incorporated by reference herein in its entirety. U.S. Pat. Nos. 5,703,436 and 5,707,745, which are incorporated by reference herein in their entireties, disclose examples of cathodes including composite cathodes having a thin layer of metal such as Mg:Ag with an overlying transparent, electrically-conductive, sputter-deposited ITO layer. The theory and use of blocking layers are described in more detail in U.S. Pat. No. 6,097,147 and U.S. Patent Application Publication No. 2003/0230980, which are incorporated by reference herein in their entireties. Examples of injection layers are provided in U.S. Patent Application Publication No. 2004/0174116, which is incorporated by reference herein in its entirety. A description of protective layers may be found in U.S. Patent Application Publication No. 2004/0174116, which is incorporated by reference herein in its entirety.

The layered structure described above is provided by way of non-limiting examples. Functional OLEDs may be achieved by combining the various layers described in different ways, or layers may be omitted entirely. It may also include other layers not specifically described. Within each layer, a single material or a mixture of multiple materials can be used to achieve optimum performance. Any functional layer may include several sublayers. For example, the emissive layer may have two layers of different emitting materials to achieve desired emission spectrum.

In one embodiment, an OLED may be described as having an “organic layer” disposed between a cathode and an anode. This organic layer may include a single layer or multiple layers.

An OLED can be encapsulated by a barrier layer. FIG. 2 schematically shows an organic light emitting device 200 without limitation. FIG. 2 differs from FIG. 1 in that the organic light emitting device include a barrier layer 102, which is above the cathode 190, to protect it from harmful species from the environment such as moisture and oxygen. Any material that can provide the barrier function can be used as the barrier layer such as glass or organic-inorganic hybrid layers. The barrier layer should be placed directly or indirectly outside of the OLED device. Multilayer thin film encapsulation was described in U.S. Pat. No. 7,968,146, which is incorporated by reference herein in its entirety.

Devices fabricated in accordance with embodiments of the present disclosure can be incorporated into a wide variety of consumer products that have one or more of the electronic component modules (or units) incorporated therein. Some examples of such consumer products include flat panel displays, monitors, medical monitors, televisions, billboards, lights for interior or exterior illumination and/or signaling, heads-up displays, fully or partially transparent displays, flexible displays, smart phones, tablets, phablets, wearable devices, smart watches, laptop computers, digital cameras, camcorders, viewfinders, micro-displays, 3-D displays, vehicles displays, and vehicle tail lights.

The materials and structures described herein may be used in other organic electronic devices listed above.

As used herein, “top” means furthest away from the substrate, while “bottom” means closest to the substrate. Where a first layer is described as “disposed over” a second layer, the first layer is disposed further away from the substrate. There may be other layers between the first and second layers, unless it is specified that the first layer is “in contact with” the second layer. For example, a cathode may be described as “disposed over” an anode, even though there are various organic layers in between.

As used herein, “solution processible” means capable of being dissolved, dispersed, or transported in and/or deposited from a liquid medium, either in solution or suspension form.

A ligand may be referred to as “photoactive” when it is believed that the ligand directly contributes to the photoactive properties of an emissive material. A ligand may be referred to as “ancillary” when it is believed that the ligand does not contribute to the photoactive properties of an emissive material, although an ancillary ligand may alter the properties of a photoactive ligand.

It is believed that the internal quantum efficiency (IQE) of fluorescent OLEDs can exceed the 25% spin statistics limit through delayed fluorescence. As used herein, there are two types of delayed fluorescence, i.e. P-type delayed fluorescence and E-type delayed fluorescence. P-type delayed fluorescence is generated from triplet-triplet annihilation (TTA).

On the other hand, E-type delayed fluorescence does not rely on the collision of two triplets, but rather on the transition between the triplet states and the singlet excited states. Compounds that are capable of generating E-type delayed fluorescence are required to have very small singlet-triplet gaps to convert between energy states. Thermal energy can activate the transition from the triplet state back to the singlet state. This type of delayed fluorescence is also known as thermally activated delayed fluorescence (TADF). A distinctive feature of TADF is that the delayed component increases as temperature rises. If the reverse intersystem crossing (RISC) rate is fast enough to minimize the non-radiative decay from the triplet state, the fraction of back populated singlet excited states can potentially reach 75%. The total singlet fraction can be 100%, far exceeding 25% of the spin statistics limit for electrically generated excitons.

E-type delayed fluorescence characteristics can be found in an exciplex system or in a single compound. Without being bound by theory, it is believed that E-type delayed fluorescence requires the luminescent material to have a small singlet-triplet energy gap (ΔE_{S-T}). Organic, non-metal containing, donor-acceptor luminescent materials may be able to achieve this. The emission in these materials is generally characterized as a donor-acceptor charge-transfer (CT) type emission. The spatial separation of the HOMO and LUMO in these donor-acceptor type compounds generally results in small ΔE_{S-T}. These states may involve CT states. Generally, donor-acceptor luminescent materials are constructed by connecting an electron donor moiety such as amino- or carbazole-derivatives and an electron acceptor moiety such as N-containing six-membered aromatic rings.

### Definition of Terms of Substituents

Halogen or halide—as used herein includes fluorine, chlorine, bromine, and iodine.

Alkyl—as used herein includes both straight and branched chain alkyl groups. Alkyl may be alkyl having 1 to 20 carbon atoms, preferably alkyl having 1 to 12 carbon atoms, and more preferably alkyl having 1 to 6 carbon atoms. Examples of alkyl groups include a methyl group, an ethyl group, a propyl group, an isopropyl group, an n-butyl group, an s-butyl group, an isobutyl group, a t-butyl group, an n-pentyl group, an n-hexyl group, an n-heptyl group, an n-octyl group, an n-nonyl group, an n-decyl group, an n-undecyl group, an n-dodecyl group, an n-tridecyl group, an n-tetradecyl group, an n-pentadecyl group, an n-hexadecyl group, an n-heptadecyl group, an n-octadecyl group, a neopentyl group, a 1-methylpentyl group, a 2-methylpentyl group, a 1-pentylhexyl group, a 1-butylpentyl group, a 1-heptyloctyl group, and a 3-methylpentyl group. Of the above, preferred are a methyl group, an ethyl group, a propyl group, an isopropyl group, a n-butyl group, an s-butyl group, an isobutyl group, a t-butyl group, an n-pentyl group, a neopentyl group, and an n-hexyl group. Additionally, the alkyl group may be optionally substituted.

Cycloalkyl—as used herein includes cyclic alkyl groups. The cycloalkyl groups may be those having 3 to 20 ring carbon atoms, preferably those having 4 to 10 carbon atoms. Examples of cycloalkyl include cyclobutyl, cyclopentyl, cyclohexyl, 4-methylcyclohexyl, 4,4-dimethylcylcohexyl, 1-adamantyl, 2-adamantyl, 1-norbornyl, 2-norbornyl, and the like. Of the above, preferred are cyclopentyl, cyclohexyl, 4-methylcyclohexyl, and 4,4-dimethylcylcohexyl. Additionally, the cycloalkyl group may be optionally substituted.

Heteroalkyl—as used herein, includes a group formed by replacing one or more carbons in an alkyl chain with a hetero-atom(s) selected from the group consisting of a nitrogen atom, an oxygen atom, a sulfur atom, a selenium atom, a phosphorus atom, a silicon atom, a germanium atom, and a boron atom. Heteroalkyl may be those having 1 to 20 carbon atoms, preferably those having 1 to 10 carbon atoms, and more preferably those having 1 to 6 carbon atoms. Examples of heteroalkyl include methoxymethyl, ethoxymethyl, ethoxyethyl, methylthiomethyl, ethylthiomethyl, ethylthioethyl, methoxymethoxymethyl, ethoxymethoxymethyl, ethoxyethoxyethyl, hydroxymethyl, hydroxyethyl, hydroxypropyl, mercaptomethyl, mercaptoethyl, mercaptopropyl, aminomethyl, aminoethyl, aminopropyl, dimethylaminomethyl, trimethylgermanylmethyl, trimethylgermanylethyl, trimethylgermanylisopropyl, dimethylethylgermanylmethyl, dimethylisopropylgermanylmethyl, tert-butylmethylgermanylmethyl, triethylgermanylmethyl, triethylgermanylethyl, triisopropylgermanylmethyl, triisopropylgermanylethyl, trimethylsilylmethyl, trimethylsilylethyl, and trimethylsilylisopropyl, triisopropylsilylmethyl, triisopropylsilylethyl. Additionally, the heteroalkyl group may be optionally substituted.

Alkenyl—as used herein includes straight chain, branched chain, and cyclic alkene groups. Alkenyl may be those having 2 to 20 carbon atoms, preferably those having 2 to 10 carbon atoms. Examples of alkenyl include vinyl, 1-propenyl group, 1-butenyl, 2-butenyl, 3-butenyl, 1,3-butandienyl, 1-methylvinyl, styryl, 2,2-diphenylvinyl, 1,2-diphenylvinyl, 1-methylallyl, 1,1-dimethylallyl, 2-methylallyl, 1-phenylallyl, 2-phenylallyl, 3-phenylallyl, 3,3-diphenylallyl, 1,2-dimethylallyl, 1-phenyl-1-butenyl, 3-phenyl-1-butenyl, cyclopentenyl, cyclopentadienyl, cyclohexenyl, cycloheptenyl, cycloheptatrienyl, cyclooctenyl, cyclooctatetraenyl, and norbornenyl. Additionally, the alkenyl group may be optionally substituted.

Alkynyl—as used herein includes straight chain alkynyl groups. Alkynyl may be those having 2 to 20 carbon atoms, preferably those having 2 to 10 carbon atoms. Examples of alkynyl groups include ethynyl, propynyl, propargyl, 1-butynyl, 2-butynyl, 3-butynyl, 1-pentynyl, 2-pentynyl, 3,3-dimethyl-1-butynyl, 3-ethyl-3-methyl-1-pentynyl, 3,3-diisopropyl-1-pentynyl, phenylethynyl, phenylpropynyl, etc. Of the above, preferred are ethynyl, propynyl, propargyl, 1-butynyl, 2-butynyl, 3-butynyl, 1-pentynyl, and phenylethynyl. Additionally, the alkynyl group may be optionally substituted.

Aryl or an aromatic group—as used herein includes non-condensed and condensed systems. Aryl may be those having 6 to 30 carbon atoms, preferably those having 6 to 20 carbon atoms, and more preferably those having 6 to 12 carbon atoms. Examples of aryl groups include phenyl, biphenyl, terphenyl, triphenylene, tetraphenylene, naphthalene, anthracene, phenalene, phenanthrene, fluorene, pyrene, chrysene, perylene, and azulene, preferably phenyl, biphenyl, terphenyl, triphenylene, fluorene, and naphthalene. Examples of non-condensed aryl groups include phenyl, biphenyl-2-yl, biphenyl-3-yl, biphenyl-4-yl, p-terphenyl-4-yl, p-terphenyl-3-yl, p-terphenyl-2-yl, m-terphenyl-4-yl, m-terphenyl-3-yl, m-terphenyl-2-yl, o-tolyl, m-tolyl, p-tolyl, p-(2-phenylpropyl)phenyl, 4′-methylbiphenylyl, 4″-t-butyl-p-terphenyl-4-yl, o-cumenyl, m-cumenyl, p-cumenyl, 2,3-xylyl, 3,4-xylyl, 2,5-xylyl, mesityl, and m-quarterphenyl. Additionally, the aryl group may be optionally substituted.

Heterocyclic groups or heterocycle—as used herein include non-aromatic cyclic groups. Non-aromatic heterocyclic groups include saturated heterocyclic groups having 3 to 20 ring atoms and unsaturated non-aromatic heterocyclic groups having 3 to 20 ring atoms, where at least one ring atom is selected from the group consisting of a nitrogen atom, an oxygen atom, a sulfur atom, a selenium atom, a silicon atom, a phosphorus atom, a germanium atom, and a boron atom. Preferred non-aromatic heterocyclic groups are those having 3 to 7 ring atoms, each of which includes at least one hetero-atom such as nitrogen, oxygen, silicon, or sulfur. Examples of non-aromatic heterocyclic groups include oxiranyl, oxetanyl, tetrahydrofuranyl, tetrahydropyranyl, dioxolanyl, dioxanyl, aziridinyl, dihydropyrrolyl, tetrahydropyrrolyl, piperidinyl, oxazolidinyl, morpholinyl, piperazinyl, oxepinyl, thiepinyl, azepinyl, and tetrahydrosilolyl. Additionally, the heterocyclic group may be optionally substituted.

Heteroaryl—as used herein, includes non-condensed and condensed hetero-aromatic groups having 1 to 5 hetero-atoms, where at least one hetero-atom is selected from the group consisting of a nitrogen atom, an oxygen atom, a sulfur atom, a selenium atom, a silicon atom, a phosphorus atom, a germanium atom, and a boron atom. A hetero-aromatic group is also referred to as heteroaryl. Heteroaryl may be those having 3 to 30 carbon atoms, preferably those having 3 to 20 carbon atoms, and more preferably those having 3 to 12 carbon atoms. Suitable heteroaryl groups include dibenzothiophene, dibenzofuran, dibenzoselenophene, furan, thiophene, benzofuran, benzothiophene, benzoselenophene, carbazole, indolocarbazole, pyridoindole, pyrrolodipyridine, pyrazole, imidazole, triazole, oxazole, thiazole, oxadiazole, oxatriazole, dioxazole, thiadiazole, pyridine, pyridazine, pyrimidine, pyrazine, triazine, oxazine, oxathiazine, oxadiazine, indole, benzimidazole, indazole, indoxazine, benzoxazole, benzisoxazole, benzothiazole, quinoline, isoquinoline, cinnoline, quinazoline, quinoxaline, naphthyridine, phthalazine, pteridine, xanthene, acridine, phenazine, phenothiazine, benzofuropyridine, furodipyridine, benzothienopyridine, thienodipyridine, benzoselenophenopyridine, and selenophenodipyridine, preferably dibenzothiophene, dibenzofuran, dibenzoselenophene, carbazole, indolocarbazole, imidazole, pyridine, triazine, benzimidazole, 1,2-azaborine, 1,3-azaborine, 1,4-azaborine, borazine, and aza-analogs thereof. Additionally, the heteroaryl group may be optionally substituted.

Alkoxy—as used herein, is represented by —O-alkyl, —O-cycloalkyl, —O-heteroalkyl, or —O-heterocyclic group. Examples and preferred examples of alkyl, cycloalkyl, heteroalkyl, and heterocyclic groups are the same as those described above. Alkoxy groups may be those having 1 to 20 carbon atoms, preferably those having 1 to 6 carbon atoms. Examples of alkoxy groups include methoxy, ethoxy, propoxy, butoxy, pentyloxy, hexyloxy, cyclopropyloxy, cyclobutyloxy, cyclopentyloxy, cyclohexyloxy, tetrahydrofuranyloxy, tetrahydropyranyloxy, methoxypropyloxy, ethoxyethyloxy, methoxymethyloxy, and ethoxymethyloxy. Additionally, the alkoxy group may be optionally substituted.

Aryloxy—as used herein, is represented by —O-aryl or —O-heteroaryl. Examples and preferred examples of aryl and heteroaryl are the same as those described above. Aryloxy groups may be those having 6 to 30 carbon atoms, preferably those having 6 to 20 carbon atoms. Examples of aryloxy groups include phenoxy and biphenyloxy. Additionally, the aryloxy group may be optionally substituted.

Arylalkyl—as used herein, contemplates alkyl substituted with an aryl group. Arylalkyl may be those having 7 to 30 carbon atoms, preferably those having 7 to 20 carbon atoms, and more preferably those having 7 to 13 carbon atoms. Examples of arylalkyl groups include benzyl, 1-phenylethyl, 2-phenylethyl, 1-phenylisopropyl, 2-phenylisopropyl, phenyl-t-butyl, alpha-naphthylmethyl, 1-alpha-naphthylethyl, 2-alpha-naphthylethyl, 1-alpha-naphthylisopropyl, 2-alpha-naphthylisopropyl, beta-naphthylmethyl, 1-beta-naphthylethyl, 2-beta-naphthylethyl, 1-beta-naphthylisopropyl, 2-beta-naphthylisopropyl, p-methylbenzyl, m-methylbenzyl, o-methylbenzyl, p-chlorobenzyl, m-chlorobenzyl, o-chlorobenzyl, p-bromobenzyl, m-bromobenzyl, o-bromobenzyl, p-iodobenzyl, m-iodobenzyl, o-iodobenzyl, p-hydroxybenzyl, m-hydroxybenzyl, o-hydroxybenzyl, p-aminobenzyl, m-aminobenzyl, o-aminobenzyl, p-nitrobenzyl, m-nitrobenzyl, o-nitrobenzyl, p-cyanobenzyl, m-cyanobenzyl, o-cyanobenzyl, 1-hydroxy-2-phenylisopropyl, and 1-chloro-2-phenylisopropyl. Of the above, preferred are benzyl, p-cyanobenzyl, m-cyanobenzyl, o-cyanobenzyl, 1-phenylethyl, 2-phenylethyl, 1-phenylisopropyl, and 2-phenylisopropyl. Additionally, the arylalkyl group may be optionally substituted.

Alkylsilyl—as used herein, contemplates a silyl group substituted with an alkyl group. Alkylsilyl groups may be those having 3 to 20 carbon atoms, preferably those having 3 to 10 carbon atoms. Examples of alkylsilyl groups include trimethylsilyl, triethylsilyl, methyldiethylsilyl, ethyldimethylsilyl, tripropylsilyl, tributylsilyl, triisopropylsilyl, methyldiisopropylsilyl, dimethylisopropylsilyl, tri-t-butylsilyl, triisobutylsilyl, dimethyl t-butylsilyl, and methyldi-t-butylsilyl. Additionally, the alkylsilyl group may be optionally substituted.

Arylsilyl—as used herein, contemplates a silyl group substituted with an aryl group. Arylsilyl groups may be those having 6 to 30 carbon atoms, preferably those having 8 to 20 carbon atoms. Examples of arylsilyl groups include triphenylsilyl, phenyldibiphenylylsilyl, diphenylbiphenylsilyl, phenyldiethylsilyl, diphenylethylsilyl, phenyldimethylsilyl, diphenylmethylsilyl, phenyldiisopropylsilyl, diphenylisopropylsilyl, diphenylbutylsilyl, diphenylisobutylsilyl, diphenyl t-butylsilyl. Additionally, the arylsilyl group may be optionally substituted.

Alkylgermanyl—as used herein contemplates germanyl substituted with an alkyl group. The alkylgermanyl may be those having 3 to 20 carbon atoms, preferably those having 3 to 10 carbon atoms. Examples of alkylgermanyl include trimethylgermanyl, triethylgermanyl, methyldiethylgermanyl, ethyldimethylgermanyl, tripropylgermanyl, tributylgermanyl, triisopropylgermanyl, methyldiisopropylgermanyl, dimethylisopropylgermanyl, tri-t-butylgermanyl, triisobutylgermanyl, dimethyl-t-butylgermanyl, and methyldi-t-butylgermanyl. Additionally, the alkylgermanyl may be optionally substituted.

Arylgermanyl—as used herein contemplates a germanyl substituted with at least one aryl group or heteroaryl group. Arylgermanyl may be those having 6 to 30 carbon atoms, preferably those having 8 to 20 carbon atoms. Examples of arylgermanyl include triphenylgermanyl, phenyldibiphenylylgermanyl, diphenylbiphenylgermanyl, phenyldiethylgermanyl, diphenylethylgermanyl, phenyldimethylgermanyl, diphenylmethylgermanyl, phenyldiisopropylgermanyl, diphenylisopropylgermanyl, diphenylbutylgermanyl, diphenylisobutylgermanyl, and diphenyl-t-butylgermanyl. Additionally, the arylgermanyl may be optionally substituted.

The term “aza” in azadibenzofuran, azadibenzothiophene, etc. means that one or more of C—H groups in the respective aromatic fragment are replaced by a nitrogen atom. For example, azatriphenylene encompasses dibenzo[f,h]quinoxaline, dibenzo[f,h]quinoline and other analogs with two or more nitrogens in the ring system. One of ordinary skill in the art can readily envision other nitrogen analogs of the aza-derivatives described above, and all such analogs are intended to be encompassed by the terms as set forth herein.

In the present disclosure, unless otherwise defined, when any term of the group consisting of substituted alkyl, substituted cycloalkyl, substituted heteroalkyl, substituted heterocyclic group, substituted arylalkyl, substituted alkoxy, substituted aryloxy, substituted alkenyl, substituted alkynyl, substituted aryl, substituted heteroaryl, substituted alkylsilyl, substituted arylsilyl, substituted alkylgermanyl, substituted arylgermanyl, substituted amino, substituted acyl, substituted carbonyl, a substituted carboxylic acid group, a substituted ester group, substituted sulfinyl, substituted sulfonyl, and substituted phosphino is used, it means that any group of alkyl, cycloalkyl, heteroalkyl, heterocyclic group, arylalkyl, alkoxy, aryloxy, alkenyl, alkynyl, aryl, heteroaryl, alkylsilyl, arylsilyl, amino, acyl, carbonyl, a carboxylic acid group, an ester group, sulfinyl, sulfonyl, and phosphino may be substituted with one or more moieties selected from the group consisting of deuterium, halogen, unsubstituted alkyl having 1 to 20 carbon atoms, unsubstituted cycloalkyl having 3 to 20 ring carbon atoms, unsubstituted heteroalkyl having 1 to 20 carbon atoms, an unsubstituted heterocyclic group having 3 to 20 ring atoms, unsubstituted arylalkyl having 7 to 30 carbon atoms, unsubstituted alkoxy having 1 to 20 carbon atoms, unsubstituted aryloxy having 6 to 30 carbon atoms, unsubstituted alkenyl having 2 to 20 carbon atoms, unsubstituted alkynyl having 2 to 20 carbon atoms, unsubstituted aryl having 6 to 30 carbon atoms, unsubstituted heteroaryl having 3 to 30 carbon atoms, unsubstituted alkylsilyl having 3 to 20 carbon atoms, unsubstituted arylsilyl having 6 to 20 carbon atoms, unsubstituted alkylgermanyl having 3 to 20 carbon atoms, unsubstituted arylgermanyl group having 6 to 20 carbon atoms, unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group, and combinations thereof.

It is to be understood that when a molecular fragment is described as being a substituent or otherwise attached to another moiety, its name may be written as if it were a fragment (e.g. phenyl, phenylene, naphthyl, dibenzofuryl) or as if it were the whole molecule (e.g. benzene, naphthalene, dibenzofuran). As used herein, these different ways of designating a substituent or an attached fragment are considered to be equivalent.

In the compounds mentioned in the present disclosure, hydrogen atoms may be partially or fully replaced by deuterium. Other atoms such as carbon and nitrogen can also be replaced by their other stable isotopes. The replacement by other stable isotopes in the compounds may be preferred due to its enhancements of device efficiency and stability.

In the compounds mentioned in the present disclosure, multiple substitution refers to a range that includes a di-substitution, up to the maximum available substitution. When substitution in the compounds mentioned in the present disclosure represents multiple substitution (including di-, tri-, and tetra-substitutions, etc.), that means the substituent may exist at a plurality of available substitution positions on its linking structure, the substituents present at a plurality of available substitution positions may be the same structure or different structures.

In the compounds mentioned in the present disclosure, adjacent substituents in the compounds cannot be joined to form a ring unless otherwise explicitly defined, for example, adjacent substituents can be optionally joined to form a ring. In the compounds mentioned in the present disclosure, the expression that adjacent substituents can be optionally joined to form a ring includes a case where adjacent substituents may be joined to form a ring and a case where adjacent substituents are not joined to form a ring. When adjacent substituents can be optionally joined to form a ring, the ring formed may be monocyclic or polycyclic (including spirocyclic, endocyclic, fused cyclic, and etc.), as well as alicyclic, heteroalicyclic, aromatic, or heteroaromatic. In such expression, adjacent substituents may refer to substituents bonded to the same atom, substituents bonded to carbon atoms which are directly bonded to each other, or substituents bonded to carbon atoms which are more distant from each other. Preferably, adjacent substituents refer to substituents bonded to the same carbon atom and substituents bonded to carbon atoms which are directly bonded to each other.

The expression that adjacent substituents can be optionally joined to form a ring is also intended to mean that two substituents bonded to the same carbon atom are joined to each other via a chemical bond to form a ring, which can be exemplified by the following formula:

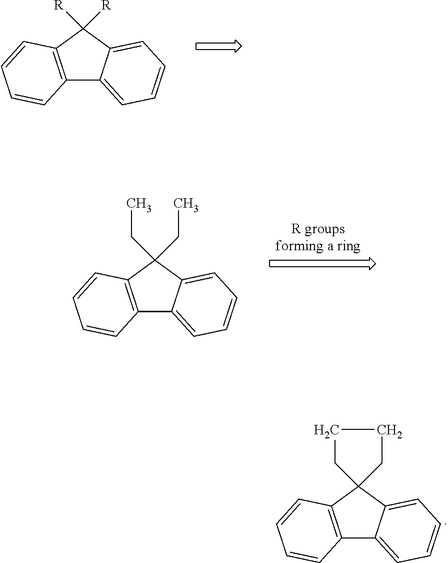

The expression that adjacent substituents can be optionally joined to form a ring is also intended to mean that two substituents bonded to carbon atoms which are directly bonded to each other are joined to each other via a chemical bond to form a ring, which can be exemplified by the following formula:

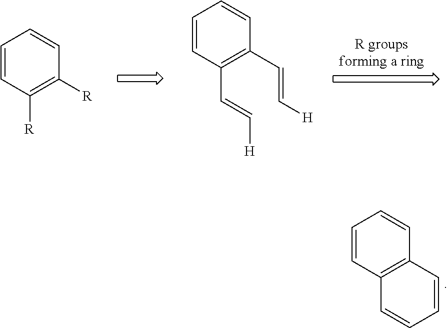

The expression that adjacent substituents can be optionally joined to form a ring is also intended to mean that two substituents bonded to a further distant carbon atom are joined to each other via a chemical bond to form a ring, which can be exemplified by the following formula:

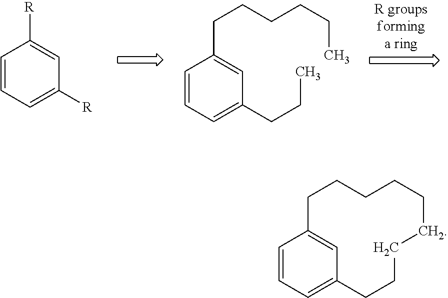

Furthermore, the expression that adjacent substituents can be optionally joined to form a ring is also intended to mean that, in the case where one of the two substituents bonded to carbon atoms which are directly bonded to each other represents hydrogen, the second substituent is bonded at a position at which the hydrogen atom is bonded, thereby forming a ring. This is exemplified by the following formula:

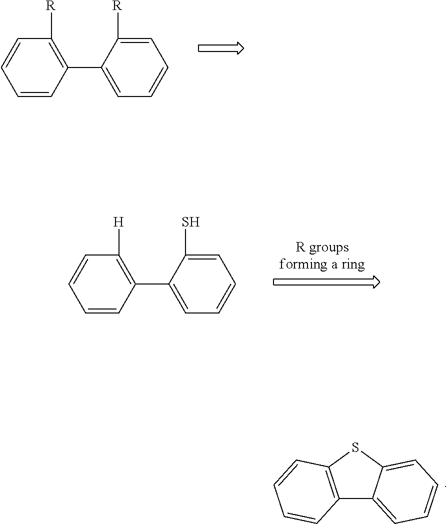

According to an embodiment of the present disclosure, disclosed is a metal complex having a general formula of M(Lₐ)ₘ(L_{b})ₙ(L_{c})_{q};

wherein

Lₐ, L_{b} and L_{c} are a first ligand, a second ligand and a third ligand coordinated to the metal M, respectively, and Lₐ, L_{b} and L_{c} are the same or different; wherein Lₐ, L_{b} and L_{c} can be optionally joined to form a multidentate ligand;

the metal M is selected from a metal with a relative atomic mass greater than 40; and

m is selected from 1 or 2, n is selected from 1 or 2, q is selected from 0 or 1, and m+n+q equals an oxidation state of M; when m is 2, two Lₐ are identical or different; when n is 2, two L_{b} are identical or different;

Lₐ has, at each occurrence identically or differently, a structure represented by Formula 1A and L_{b} has, at each occurrence identically or differently, a structure represented by Formula 1B:

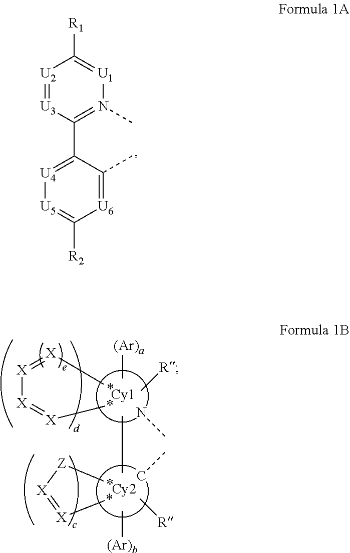

wherein

the ring Cy1 is, at each occurrence identically or differently, selected from a heteroaromatic ring having 5 to 6 ring atoms;

the ring Cy2 is, at each occurrence identically or differently, selected from a benzene ring or a heteroaromatic ring having 5 to 6 ring atoms;

Ar is, at each occurrence identically or differently, selected from substituted or unsubstituted aryl having 6 to 30 carbon atoms, substituted or unsubstituted heteroaryl having 3 to 30 carbon atoms or a combination thereof;

a, b, c, d and e are, at each occurrence identically or differently, selected from 0 or 1, and at least one of a, b, c and d is selected from 1;

Z is, at each occurrence identically or differently, selected from O or X═X;

X is, at each occurrence identically or differently, selected from N or CR′;

U₁ to U₆ are, at each occurrence identically or differently, selected from CRᵤ or N;

R″ is, at each occurrence identically or differently, selected from mono-substitution, multiple substitutions or non-substitution;

R′, R″ and Rᵤ are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group and combinations thereof;

“*” represents a position where the ring Cy1 or the ring Cy2 is joined;

in Formula 1A, adjacent substituents Rᵤ can be optionally joined to form a ring;

in Formula 1B, adjacent substituents R′ and R″ can be optionally joined to form a ring; and

R₁ is, at each occurrence identically or differently, selected from the group consisting of: substituted or unsubstituted alkyl having 4 to 20 carbon atoms, substituted or unsubstituted cycloalkyl having 4 to 20 ring carbon atoms, substituted or unsubstituted heteroalkyl having 4 to 20 carbon atoms, a substituted or unsubstituted heterocyclic group having 4 to 20 ring atoms, substituted or unsubstituted arylalkyl having 7 to 30 carbon atoms, substituted or unsubstituted alkoxy having 4 to 20 carbon atoms, substituted or unsubstituted aryloxy having 6 to 30 carbon atoms, substituted or unsubstituted alkenyl having 4 to 20 carbon atoms, substituted or unsubstituted alkynyl having 4 to 20 carbon atoms, substituted or unsubstituted aryl having 6 to 30 carbon atoms, substituted or unsubstituted heteroaryl having 4 to 30 carbon atoms and combinations thereof;

R₂ has a structure represented by Formula 2:

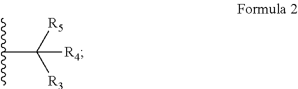

wherein

the number of carbon atoms in R₂ is greater than or equal to 4;

R₃, R₄ and R₅ are, at each occurrence identically or differently, selected from the group consisting of: halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group and combinations thereof;

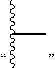

represents a position where Formula 2 is joined to Formula 1A;

two adjacent substituents of R₃, R₄ and R₅ can be optionally joined to form a ring; and

L_{c} is a mono anionic bidentate ligand.

In the present disclosure, the expression “adjacent substituents R′ and R″ can be optionally joined to form a ring” is intended to mean that any one or more of groups of adjacent substituents, such as two adjacent substituents R′, and two adjacent substituents R″, can be joined to form a ring. Obviously, it is possible that none of these substituents are joined to form a ring.

In the present disclosure, the expression that “adjacent substituents Rᵤ can be optionally joined to form a ring” is intended to mean that when more than one of U₁ to U₆ are selected from CRᵤ, any one or more of groups consisting of any two adjacent substituents Rᵤ can be joined to form a ring. Obviously, it is possible that none of these substituents are joined to forma ring.

In the present disclosure, the expression that “two adjacent substituents of R₃, R₄ and R₅ can be optionally joined to form a ring” is intended to mean that any one or more of groups of adjacent substituents, such as substituents R₃ and R₄, substituents R₄ and R₅, and substituents R₃ and R₅, can be joined to form a ring. Obviously, it is possible that none of these substituents are joined to form a ring.

In the present disclosure, “a, b, c, d and e are, at each occurrence identically or differently, selected from 0 or 1” is intended to mean that when a to e are selected from 0, relevant substituents or groups are absent; when a to e are selected from 1, relevant substituents or groups are present. For example, when a is 0, it means that the ring Cy1 does not have the substituent Ar; when a is 1, it means that the ring Cy1 must have the substituent Ar. Similarly, other cases are explained in the same manner.

In the present disclosure, “Z is, at each occurrence identically or differently, selected from O or X═X” is intended to mean that

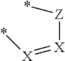

has the following two structures:

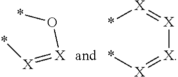

The two “*” in the ring Cy2 means that when c is 1, at least two adjacent C are present in the ring Cy2, and the ring Cy2 and

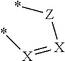

can form any one of the following structures:

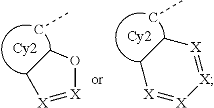

when c is 0, the ring Cy2 is not joined to

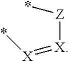

In the present disclosure, “e is, at each occurrence identically or differently, selected from 0 or 1” is intended to mean that when e is 0 or 1,

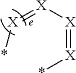

has the structure

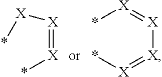

respectively. The two “*” in the ring Cy1 means that when d is 1, at least two adjacent C are present in the ring Cy1, and the ring Cy1 is separately joined to

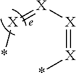

to form the following structures:

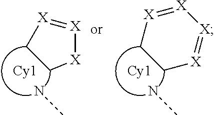

when d is 0, the ring Cy1 is not joined to

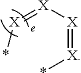

According to an embodiment of the present disclosure, L_{c} is, at each occurrence identically or differently, selected from a structure represented by any one of the group consisting of the following:

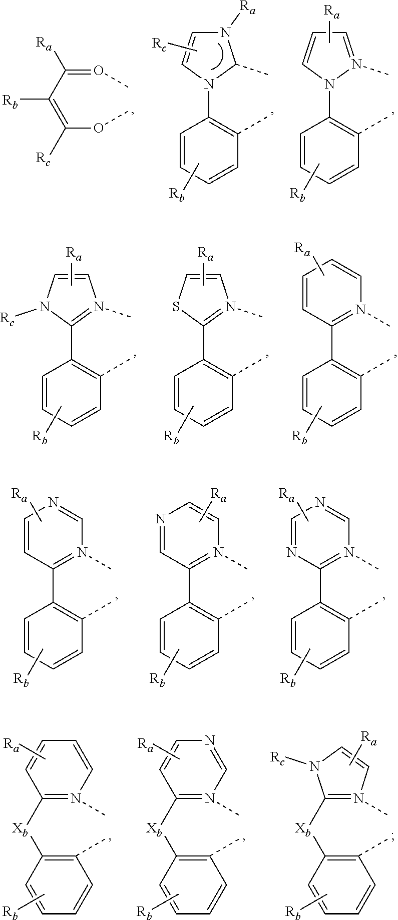

wherein

Rₐ, R_{b} and R_{c} represent, at each occurrence identically or differently, mono-substitution, multiple substitutions or non-substitution;

X_{b} is, at each occurrence identically or differently, selected from the group consisting of O, S, Se, NR_{N1} and CR_{C1}R_{C2};

Rₐ, R_{b}, R_{c}, R_{N1}, R_{C1} and R_{C2} are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group and combinations thereof; and adjacent substituents Rₐ, R_{b}, R_{N1}, R_{C1} and R_{C2} can be optionally joined to form a ring.

In the present disclosure, the expression that “adjacent substituents Rₐ, R_{b}, R_{N1}, R_{C1} and R_{C2} can be optionally joined to form a ring” is intended to mean that any one or more of groups of adjacent substituents, such as two substituents Rₐ, two substituents R_{b}, two substituents Rₐ and R_{b}, substituents Rₐ and R_{c}, substituents R_{b} and R_{c}, substituents Rₐ and R_{N1}, substituents R_{b} and R_{N1}, substituents Rₐ and R_{C1}, substituents Rₐ and R_{C2}, substituents R_{b} and R_{C1}, substituents R_{b} and R_{C2}, and substituents R_{C1} and R_{C2}, can be joined to form a ring. Obviously, it is possible that none of these substituents are joined to form a ring.

According to an embodiment of the present disclosure, the ring Cy1 is, at each occurrence identically or differently, selected from any structure of the group consisting of the following:

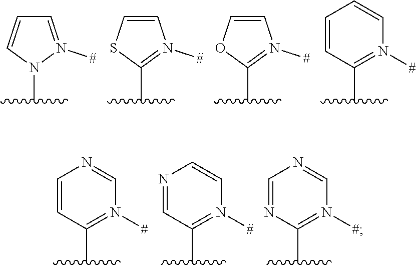

wherein “#” represents a position where the ring Cy1 is joined to the metal M, and

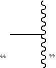

represents a position where the ring Cy1 is joined to the ring Cy2.

According to an embodiment of the present disclosure, the ring Cy2 is, at each occurrence identically or differently, selected from any structure of the group consisting of the following:

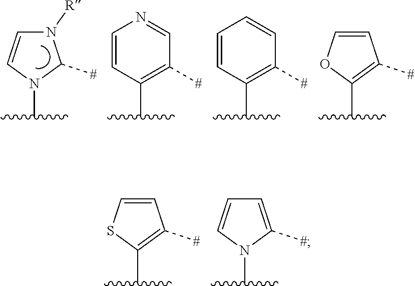

wherein “#” represents a position where the ring Cy2 is joined to the metal M, and

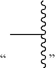

represents a position where the ring Cy2 is joined to the ring Cy1.

According to an embodiment of the present disclosure, L_{b} has a structure represented by any one of Formulas 1Ba to 1Bm:

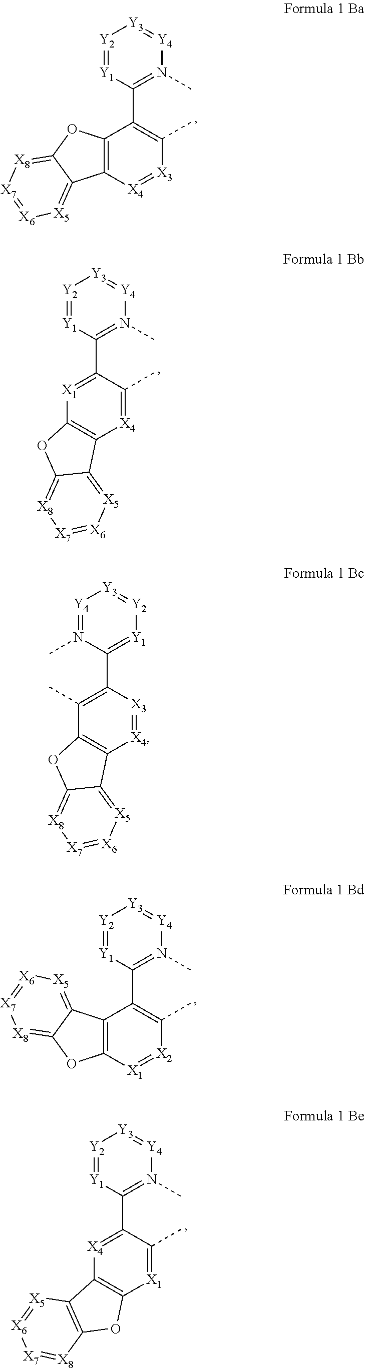

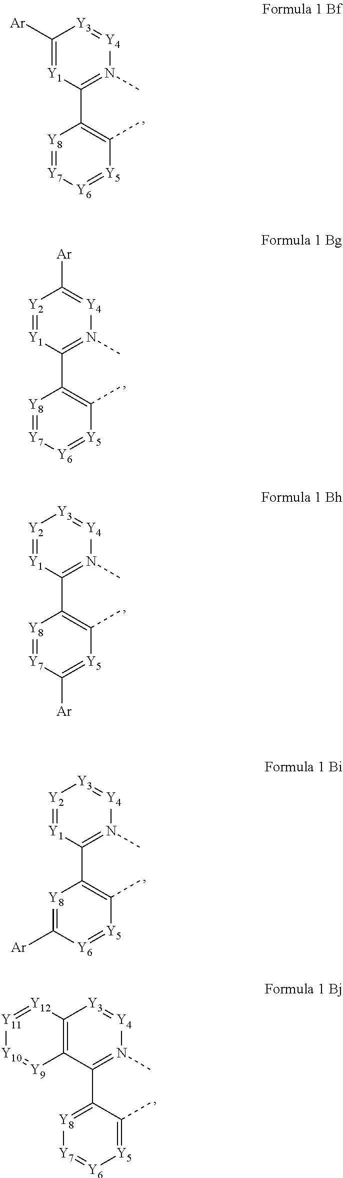

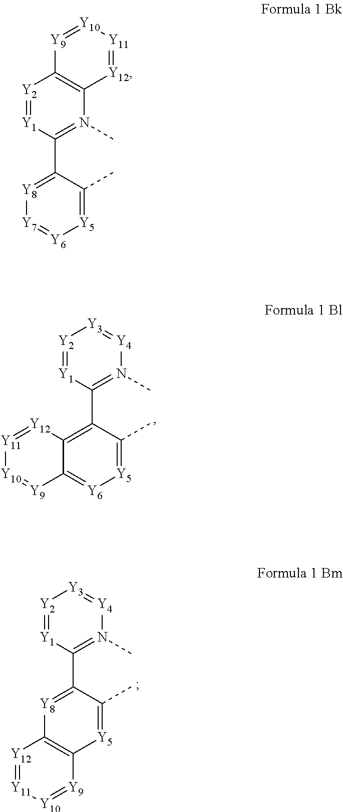

wherein

X₁ to X₈ are, at each occurrence identically or differently, selected from CRₓ or N;

Y₁ to Y₁₂ are, at each occurrence identically or differently, selected from CR_{y} or N;

Ar is, at each occurrence identically or differently, selected from substituted or unsubstituted aryl having 6 to 30 carbon atoms, substituted or unsubstituted heteroaryl having 3 to 30 carbon atoms or a combination thereof;

Rₓ and R_{y} are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group and combinations thereof; and

adjacent substituents Rₓ and R_{y} can be optionally joined to form a ring.

In the present disclosure, the expression that “adjacent substituents Rₓ and R_{y} can be optionally joined to form a ring” is intended to mean that any one or more of groups of adjacent substituents, such as two substituents Rₓ, two substituents R_{y}, and substituents Rₓ and R_{y}, can be joined to form a ring. Obviously, it is possible that none of these substituents are joined to form a ring.

According to an embodiment of the present disclosure, L_{b} has, at each occurrence identically or differently, a structure represented by any one of Formulas 1Ba to 1Bi.

According to an embodiment of the present disclosure, Ar is, at each occurrence identically or differently, selected from substituted or unsubstituted aryl having 6 to 24 carbon atoms, substituted or unsubstituted heteroaryl having 3 to 24 carbon atoms or a combination thereof.

According to an embodiment of the present disclosure, Ar is, at each occurrence identically or differently, selected from substituted or unsubstituted aryl having 6 to 18 carbon atoms, substituted or unsubstituted heteroaryl having 3 to 18 carbon atoms or a combination thereof.

According to an embodiment of the present disclosure, Ar is, at each occurrence identically or differently, selected from substituted or unsubstituted aryl having 6 to 12 carbon atoms, substituted or unsubstituted heteroaryl having 3 to 12 carbon atoms or a combination thereof.

According to an embodiment of the present disclosure, Ar is, at each occurrence identically or differently, selected from substituted or unsubstituted phenyl, substituted or unsubstituted biphenyl, substituted or unsubstituted pyridyl or a combination thereof.

According to an embodiment of the present disclosure, R₁ is, at each occurrence identically or differently, selected from the group consisting of: substituted or unsubstituted alkyl having 4 to 20 carbon atoms, substituted or unsubstituted cycloalkyl having 4 to 20 ring carbon atoms, substituted or unsubstituted heteroalkyl having 4 to 20 carbon atoms, a substituted or unsubstituted heterocyclic group having 4 to 20 ring atoms and combinations thereof.

According to an embodiment of the present disclosure, R₁ is, at each occurrence identically or differently, selected from the group consisting of: substituted or unsubstituted alkyl having 4 to 10 carbon atoms, substituted or unsubstituted cycloalkyl having 4 to 10 ring carbon atoms and combinations thereof.

According to an embodiment of the present disclosure, R₁ has a structure represented by Formula 2.

According to an embodiment of the present disclosure, wherein the metal M is, at each occurrence identically or differently, selected from the group consisting of Cu, Ag, Au, Ru, Rh, Pd, Os, Ir and Pt.

According to an embodiment of the present disclosure, wherein the metal M is, at each occurrence identically or differently, selected from Pt or Ir.

According to an embodiment of the present disclosure, R₃, R₄ and R₅ are, at each occurrence identically or differently, selected from the group consisting of: halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkenyl having 2 to 20 carbon atoms, substituted or unsubstituted aryl having 6 to 30 carbon atoms or substituted or unsubstituted heteroaryl having 3 to 30 carbon atoms.

According to an embodiment of the present disclosure, R₃, R₄ and R₅ are, at each occurrence identically or differently, selected from the group consisting of: halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms and combinations thereof.

According to an embodiment of the present disclosure, R₃, R₄ and R₅ are, at each occurrence identically or differently, selected from the group consisting of: halogen, substituted or unsubstituted alkyl having 1 to 6 carbon atoms, substituted or unsubstituted cycloalkyl having 3 to 6 ring carbon atoms and combinations thereof.

According to an embodiment of the present disclosure, the structure represented by Formula 2 represents, at each occurrence identically or differently, any one of the following structures:

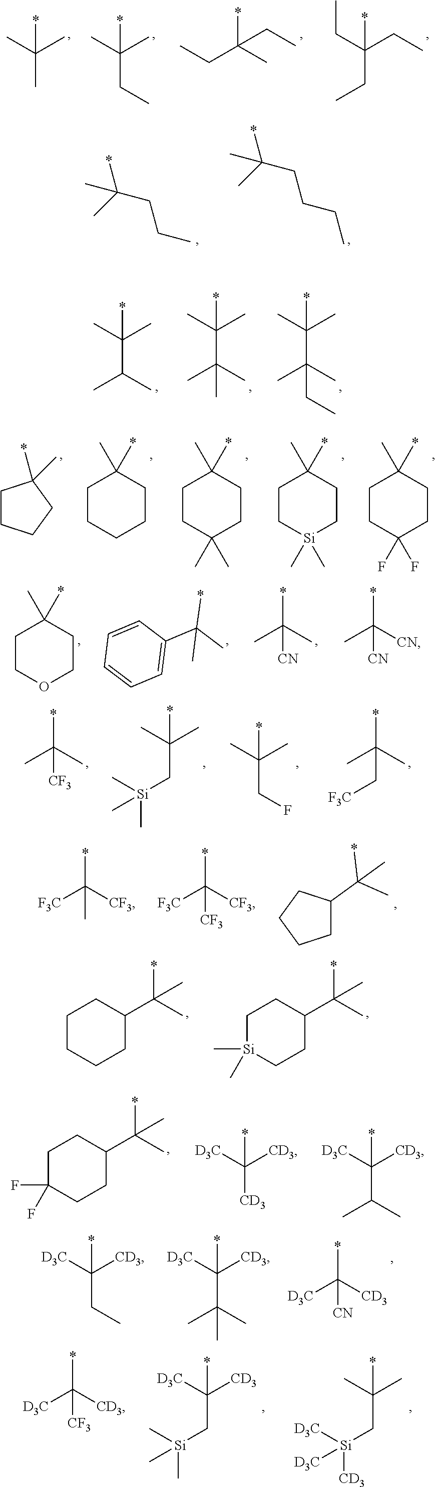

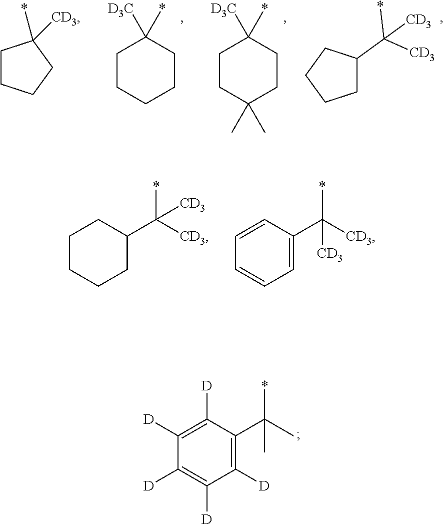

wherein “*” represents a position where the structure is joined to Formula 1A; and

optionally, hydrogen atoms in the above structures can be partially or fully substituted with deuterium atoms.

According to an embodiment of the present disclosure, the metal complex has a structure of a general formula of Ir(Lₐ)ₘ(L_{b})₃₋ₘ which is represented by Formula 3, Formula 4 or Formula 5:

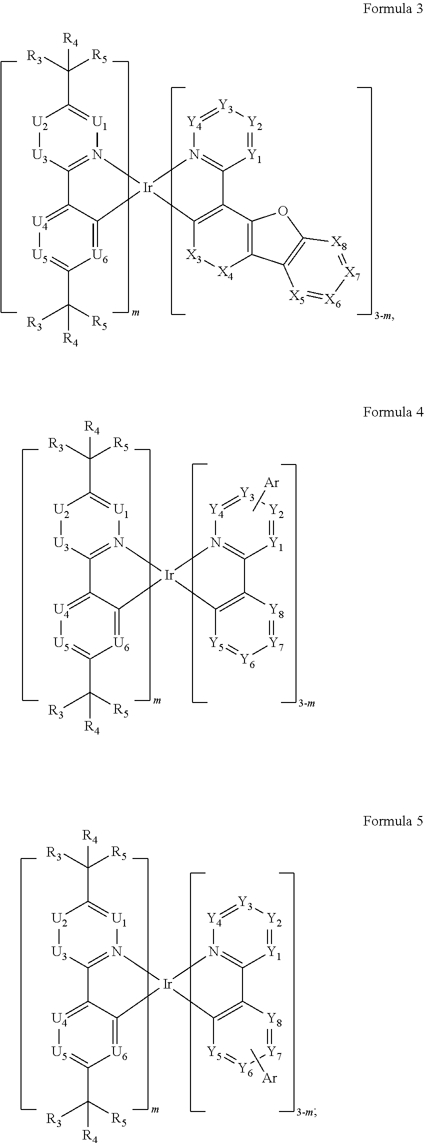

wherein

m is selected from 1 or 2; when m=1, two L_{b} are identical or different; when m=2, two Lₐ are identical or different;

X₃ to X₈ are, at each occurrence identically or differently, selected from CRₓ or N;

Y₁ to Y₈ are, at each occurrence identically or differently, selected from C, CR_{y} or N;

U₁ to U₆ are, at each occurrence identically or differently, selected from CRᵤ or N;

Ar is, at each occurrence identically or differently, selected from substituted or unsubstituted aryl having 6 to 30 carbon atoms, substituted or unsubstituted heteroaryl having 3 to 30 carbon atoms or a combination thereof;

Rₓ, R_{y} and Rᵤ are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group and combinations thereof;

R₃, R₄ and R₅ are, at each occurrence identically or differently, selected from the group consisting of: substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkenyl having 2 to 20 carbon atoms, substituted or unsubstituted aryl having 6 to 30 carbon atoms or substituted or unsubstituted heteroaryl having 3 to 30 carbon atoms;

adjacent substituents Rₓ and R_{y} can be optionally joined to form a ring; and

two adjacent substituents of R₃, R₄ and R₅ can be optionally joined to form a ring.

According to an embodiment of the present disclosure, the metal complex has a structure of the general formula of Ir(Lₐ)ₘ(L_{b})₃₋ₘ which is represented by Formula 6, Formula 7, Formula 8 or Formula 9:

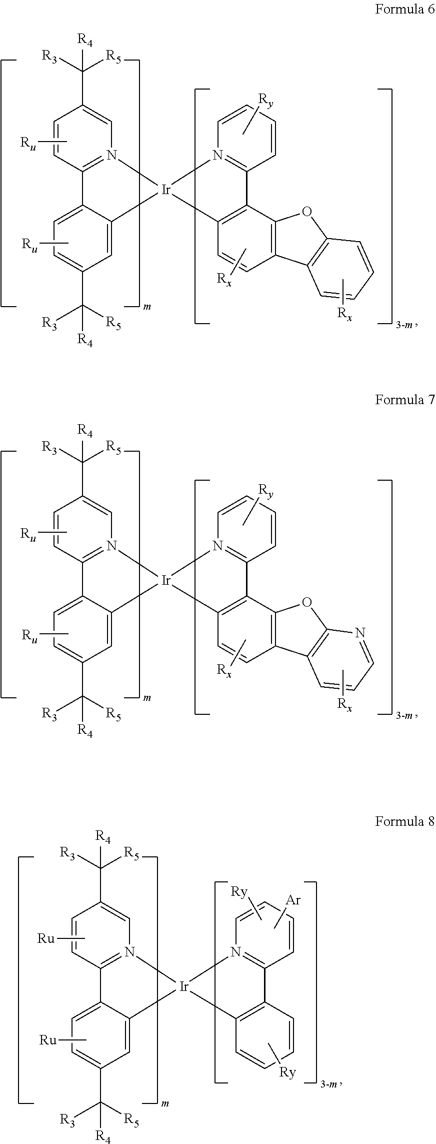

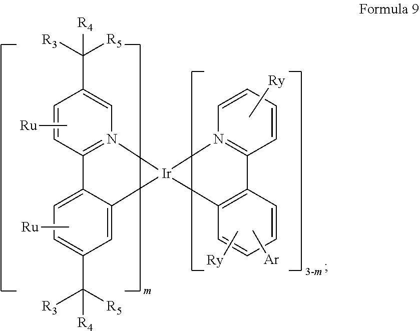

wherein

m is selected from 1 or 2; when m=1, two L_{b} are identical or different; when m=2, two Lₐ are identical or different;

Rₓ, R_{y} and Rᵤ represent, at each occurrence identically or differently, mono-substitution, multiple substitutions or non-substitution;

Ar is, at each occurrence identically or differently, selected from substituted or unsubstituted aryl having 6 to 30 carbon atoms, substituted or unsubstituted heteroaryl having 3 to 30 carbon atoms or a combination thereof;

Rₓ, R_{y} and Rᵤ are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group and combinations thereof;

R₃, R₄ and R₅ are, at each occurrence identically or differently, selected from the group consisting of: substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkenyl having 2 to 20 carbon atoms, substituted or unsubstituted aryl having 6 to 30 carbon atoms or substituted or unsubstituted heteroaryl having 3 to 30 carbon atoms;

adjacent substituents Rₓ and R_{y} can be optionally joined to form a ring; and

two adjacent substituents of R₃, R₄ and R₅ can be optionally joined to form a ring.

According to an embodiment of the present disclosure, Rₓ is, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted amino having 0 to 20 carbon atoms, a cyano group, a hydroxyl group, a sulfanyl group and combinations thereof.

According to an embodiment of the present disclosure, Rₓ is, at each occurrence identically or differently, selected from the group consisting of: hydrogen, deuterium, halogen, substituted or unsubstituted alkyl having 1 to 20 carbon atoms, substituted or unsubstituted heteroalkyl having 3 to 20 ring carbon atoms, substituted or unsubstituted aryl having 6 to 30 carbon atoms, substituted or unsubstituted heteroaryl having 3 to 30 carbon atoms, a cyano group and combinations thereof.

According to an embodiment of the present disclosure, at least one of X₃ to X₈ is selected from CRₓ, and Rₓ is selected from cyano or fluorine.

According to an embodiment of the present disclosure, at least one of X₅ to X₈ is selected from CRₓ, and Rₓ is selected from cyano or fluorine.

According to an embodiment of the present disclosure, X₇ or X₈ is selected from CRₓ, and Rₓ is selected from cyano or fluorine.

According to an embodiment of the present disclosure, at least two of X₃ to X₈ are selected from CRₓ, wherein one Rₓ is selected from cyano or fluorine, and at least another one Rₓ is selected from the group consisting of: deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group and combinations thereof.

According to an embodiment of the present disclosure, at least two of X₅ to X₈ are selected from CRₓ, wherein one Rₓ is selected from cyano or fluorine, and at least another one Rₓ is selected from the group consisting of: deuterium, halogen, substituted or unsubstituted alkyl having 1 to 20 carbon atoms, substituted or unsubstituted cycloalkyl having 3 to 20 ring carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted amino having 0 to 20 carbon atoms, a cyano group, a hydroxyl group, a sulfanyl group and combinations thereof.

According to an embodiment of the present disclosure, X₇ and X₈ are selected from CRₓ, wherein one Rₓ is cyano, and another one Rₓ is selected from the group consisting of: deuterium, halogen, substituted or unsubstituted alkyl having 1 to 20 carbon atoms, substituted or unsubstituted cycloalkyl having 3 to 20 ring carbon atoms, substituted or unsubstituted aryl having 6 to 30 carbon atoms, substituted or unsubstituted heteroaryl having 3 to 30 carbon atoms and combinations thereof.

According to an embodiment of the present disclosure, U₁ to U₆ are, at each occurrence identically or differently, selected from CRᵤ.

According to an embodiment of the present disclosure, X₁ to X₈ are, at each occurrence identically or differently, selected from CRₓ.

According to an embodiment of the present disclosure, X₃ to X₈ are, at each occurrence identically or differently, selected from CRₓ.

According to an embodiment of the present disclosure, Y₁ to Y₄ are, at each occurrence identically or differently, selected from CR_{y}.

According to an embodiment of the present disclosure, Rᵤ is, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted aryl having 6 to 30 carbon atoms, substituted or unsubstituted heteroaryl having 3 to 30 carbon atoms and combinations thereof.

According to an embodiment of the present disclosure, Rᵤ is, at each occurrence identically or differently, selected from the group consisting of: hydrogen, deuterium, substituted or unsubstituted alkyl having 1 to 10 carbon atoms, substituted or unsubstituted cycloalkyl having 3 to 10 ring carbon atoms, substituted or unsubstituted aryl having 6 to 10 carbon atoms and combinations thereof.

According to an embodiment of the present disclosure, Rᵤ is, at each occurrence identically or differently, selected from the group consisting of: hydrogen, deuterium, substituted or unsubstituted alkyl having 1 to 10 carbon atoms, substituted or unsubstituted cycloalkyl having 3 to 10 ring carbon atoms and combinations thereof.

According to an embodiment of the present disclosure, R_{y} is, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted aryl having 6 to 30 carbon atoms, substituted or unsubstituted heteroaryl having 3 to 30 carbon atoms and combinations thereof.

According to an embodiment of the present disclosure, R_{y} is, at each occurrence identically or differently, selected from the group consisting of: hydrogen, deuterium, substituted or unsubstituted alkyl having 1 to 10 carbon atoms, substituted or unsubstituted cycloalkyl having 3 to 10 ring carbon atoms, substituted or unsubstituted aryl having 6 to 10 carbon atoms and combinations thereof.

According to an embodiment of the present disclosure, R_{y} is, at each occurrence identically or differently, selected from the group consisting of: hydrogen, deuterium, substituted or unsubstituted alkyl having 1 to 10 carbon atoms, substituted or unsubstituted cycloalkyl having 3 to 10 ring carbon atoms and combinations thereof.

According to an embodiment of the present disclosure, R′ and R″ are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted alkenyl having 2 to 20 carbon atoms, substituted or unsubstituted aryl having 6 to 30 carbon atoms, substituted or unsubstituted heteroaryl having 3 to 30 carbon atoms and combinations thereof.

According to an embodiment of the present disclosure, R′ and R″ are, at each occurrence identically or differently, selected from the group consisting of: hydrogen, deuterium, substituted or unsubstituted alkyl having 1 to 10 carbon atoms, substituted or unsubstituted cycloalkyl having 3 to 10 ring carbon atoms, substituted or unsubstituted alkenyl having 2 to 10 carbon atoms, substituted or unsubstituted aryl having 6 to 10 carbon atoms and combinations thereof.

According to an embodiment of the present disclosure, R′ and R″ are, at each occurrence identically or differently, selected from the group consisting of: hydrogen, deuterium, substituted or unsubstituted alkyl having 1 to 10 carbon atoms, substituted or unsubstituted alkenyl having 2 to 10 carbon atoms, substituted or unsubstituted cycloalkyl having 3 to 10 ring carbon atoms and combinations thereof.

According to an embodiment of the present disclosure, at least one of U₁ to U₃ is selected from N, for example, one of U₁ to U₃ is selected from N, or two of U₁ to U₃ are selected from N.

According to an embodiment of the present disclosure, at least one of U₄ to U₆ is selected from N, for example, one of U₄ to U₆ is selected from N, or two of U₄ to U₆ are selected from N.

According to an embodiment of the present disclosure, at least one of Y₁ to Y₄ is selected from N, for example, one of Y₁ to Y₄ is selected from N, or two of Y₁ to Y₄ are selected from N.

According to an embodiment of the present disclosure, at least one of X₃ to X₈ is selected from N, for example, one of X₃ to X₈ is selected from N, or two of X₃ to X₈ are selected from N.

According to an embodiment of the present disclosure, Lₐ is, at each occurrence identically or differently, selected from the group consisting of Lₐ₁₋₁ to Lₐ₁₋₂₃₁, Lₐ₂₋₁ to Lₐ₂₋₁₆₁ and Lₐ₃₋₁ to Lₐ₃₋₁₃₀, wherein the specific structures of Lₐ₁₋₁ to Lₐ₁₋₂₃₁, Lₐ₂₋₁ to Lₐ₂₋₁₆₁ and Lₐ₁₋₁ to Lₐ₃₋₁₃₀ are referred to claim 17.

According to an embodiment of the present disclosure, hydrogen atoms in Lₐ₁₋₁ to Lₐ₁₋₂₃₁, Lₐ₂₋₁ to Lₐ₂₋₁₆₁ and Lₐ₃₋₁ to Lₐ₃₋₁₃₀ can be partially or fully substituted with deuterium atoms.

According to an embodiment of the present disclosure, L_{b} is, at each occurrence identically or differently, selected from the group consisting of L_{b1-1} to L_{b1-355}, L_{b2-1} to L_{b2-261} and L_{b34} to L_{b3-650}, wherein the specific structures of L_{b1-1} to L_{b1-355}, L_{b2-1} to L_{b2-261} and L_{b3-1} to L_{b3-650} are referred to claim 18.

According to an embodiment of the present disclosure, hydrogen atoms in L_{b1-1} to L_{b1-355}, L_{b2-1} to L_{b2-261} and L_{b34} to L_{b3-650} can be partially or fully substituted with deuterium atoms.

According to an embodiment of the present disclosure, L_{c} is, at each occurrence identically or differently, selected from the group consisting of L_{c1} to L_{c360}, wherein the specific structures of L_{c1} to L_{c360} are referred to claim 19.

According to an embodiment of the present disclosure, the metal complex has a structure of Ir(Lₐ)₂L_{b}, wherein the two Lₐ are identical or different, Lₐ is, at each occurrence identically or differently, selected from the group consisting of Lₐ₁₋₁ to Lₐ₁₋₂₃₁, Lₐ₂₋₁ to Lₐ₂₋₁₆₁ and Lₐ₃₋₁ to Lₐ₃₋₁₃₀, and L_{b} is selected from the group consisting of L_{b1-1} to L_{b1-355}, L_{b2-1} to L_{b2-261} and L_{b3-1} to L_{b3-650}.

According to an embodiment of the present disclosure, the metal complex has a structure of IrLₐ(L_{b})₂, wherein the two L_{b} are identical or different, Lₐ is selected from the group consisting of Lₐ₁₋₁ to Lₐ₁₋₂₃₁, Lₐ₂₋₁ to Lₐ₂₋₁₆₁ and Lₐ₃₋₁ to Lₐ₃₋₁₃₀, and L_{b} is, at each occurrence identically or differently, selected from the group consisting of L_{b1-1} to L_{b1-355}, L_{b2-1} to L_{b2-261} and L_{b3-1} to L_{b3-650}.

According to an embodiment of the present disclosure, the metal complex has a structure of Ir(Lₐ)(L_{b})(L_{c}), wherein Lₐ is selected from the group consisting of Lₐ₁₋₁ to Lₐ₁₋₂₃₁, Lₐ₂₋₁ to Lₐ₁₋₁₆₁ and Lₐ₃₋₁ to Lₐ₃₋₁₃₀, L_{b} is selected from the group consisting of L_{b1-1} to L_{b1-355}, L_{b2-1} to L_{b2-261} and L_{b3-1} to L_{b3-650}, and L_{c} is selected from the group consisting of L_{c1} to L_{c360}.

According to an embodiment of the present disclosure, the metal complex is selected from the group consisting of Metal Complex 1 to Metal Complex 2128, wherein the specific structures of Metal Complex 1 to Metal Complex 2128 are referred to claim 20.

According to an embodiment of the present disclosure, further disclosed is an electroluminescent device. The electroluminescent device includes:

an anode,

a cathode, and

an organic layer disposed between the anode and the cathode, wherein at least one layer of the organic layer comprises the metal complex according to any one of the preceding embodiments.

According to an embodiment of the present disclosure, the organic layer comprising the metal complex in the electroluminescent device is an emissive layer.

According to an embodiment of the present disclosure, the electroluminescent device emits green light.

According to an embodiment of the present disclosure, the emissive layer of the electroluminescent device further comprises a first host compound.

According to an embodiment of the present disclosure, the emissive layer of the electroluminescent device further comprises a first host compound and a second host compound.

According to an embodiment of the present disclosure, the first host compound and/or the second host compound in the electroluminescent device comprises at least one chemical group selected from the group consisting of: benzene, pyridine, pyrimidine, triazine, carbazole, azacarbazole, indolocarbazole, dibenzothiophene, azadibenzothiophene, dibenzofuran, azadibenzofuran, dibenzoselenophene, triphenylene, azatriphenylene, fluorene, silafluorene, naphthalene, quinoline, isoquinoline, quinazoline, quinoxaline, phenanthrene, azaphenanthrene, and combinations thereof.

According to an embodiment of the present disclosure, the first host compound in the electroluminescent device has a structure represented by Formula X:

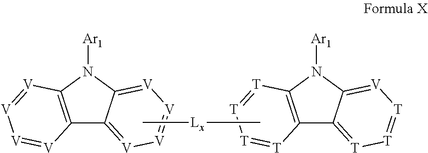

wherein,

Lₓ is, at each occurrence identically or differently, selected from a single bond, substituted or unsubstituted alkylene having 1 to 20 carbon atoms, substituted or unsubstituted cycloalkylene having 3 to 20 carbon atoms, substituted or unsubstituted arylene having 6 to 20 carbon atoms, substituted or unsubstituted heteroarylene having 3 to 20 carbon atoms or combinations thereof;

V is, at each occurrence identically or differently, selected from C, CRᵥ or N, and at least one of V is C and is attached to Lₓ;

T is, at each occurrence identically or differently, selected from C, CRₜ or N, and at least one of T is C and is attached to Lₓ;

Rᵥ and Rₜ are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group, and combinations thereof;

Ar₁ is, at each occurrence identically or differently, selected from substituted or unsubstituted aryl having 6 to 30 carbon atoms, substituted or unsubstituted heteroaryl having 3 to 30 carbon atoms or combinations thereof;

adjacent substituents Rᵥ and Rₜ can be optionally joined to form a ring.

Herein, the expression that “adjacent substituents Rᵥ and Rₜ can be optionally joined to form a ring” is intended to mean that any one or more of groups of adjacent substituents, such as two substituents Rᵥ, two substituents Rₜ, and substituents Rᵥ and Rₜ, can be joined to form a ring. Obviously, it is possible that none of these substituents are joined to form a ring. Obviously, it is possible that none of these substituents are joined to form a ring.

According to an embodiment of the present disclosure, the first host compound in the electroluminescent device has a structure represented by one of Formula X-a to Formula X-j:

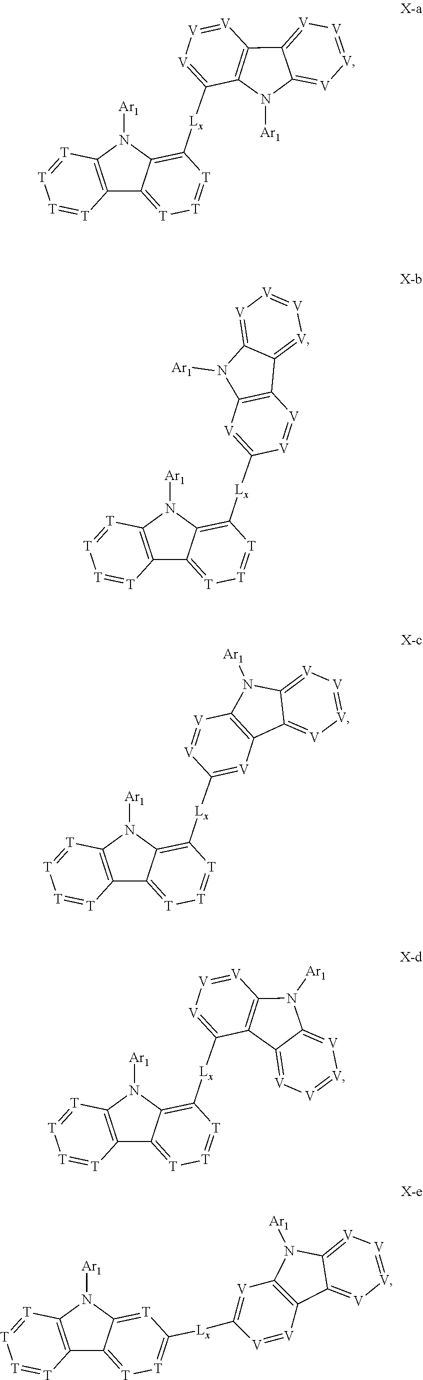

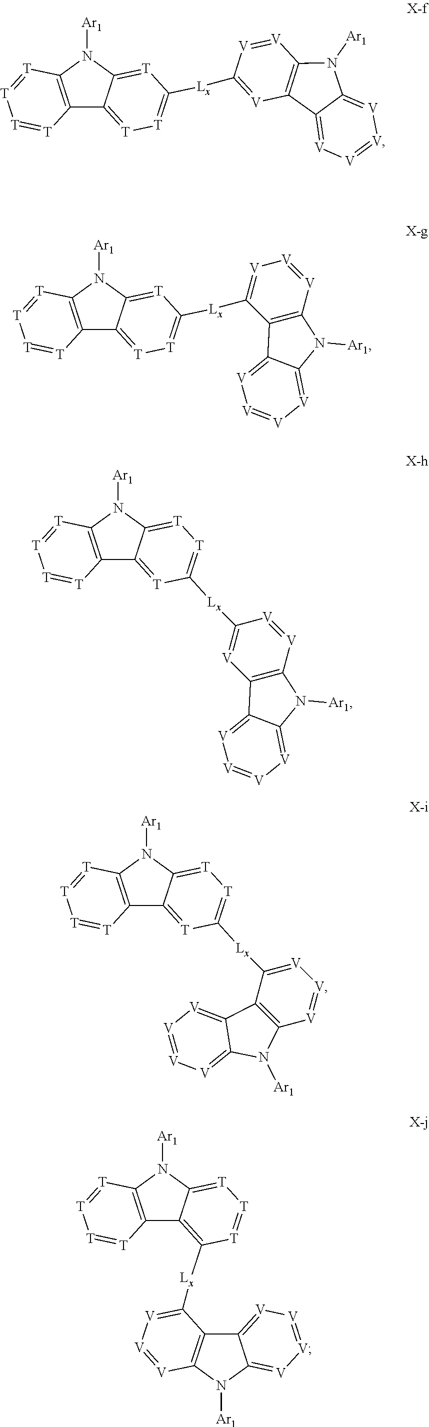

wherein,

Lₓ is, at each occurrence identically or differently, selected from a single bond, substituted or unsubstituted alkylene having 1 to 20 carbon atoms, substituted or unsubstituted cycloalkylene having 3 to 20 carbon atoms, substituted or unsubstituted arylene having 6 to 20 carbon atoms, substituted or unsubstituted heteroarylene having 3 to 20 carbon atoms or combinations thereof;

V is, at each occurrence identically or differently, selected from CRᵥ or N;

T is, at each occurrence identically or differently, selected from CRₜ or N;

Rᵥ and Rₜ are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group, and combinations thereof;

Ar₁ is, at each occurrence identically or differently, selected from substituted or unsubstituted aryl having 6 to 30 carbon atoms, substituted or unsubstituted heteroaryl having 3 to 30 carbon atoms or combinations thereof;

adjacent substituents Rᵥ and Rₜ can be optionally joined to form a ring.

According to an embodiment of the present disclosure, in the electroluminescent device, the metal complex is doped in the first host compound and the second host compound, and the weight of the metal complex accounts for 1% to 30% of the total weight of the emissive layer.

According to an embodiment of the present disclosure, in the electroluminescent device, the metal complex is doped in the first host compound and the second host compound, and the weight of the metal complex accounts for 3% to 13% of the total weight of the emissive layer.

According to another embodiment of the present disclosure, a compound composition is further disclosed. The compound composition comprises the metal complex described in any one of the above-mentioned embodiments.

Combination with Other Materials

The materials described in the present disclosure for a particular layer in an organic light-emitting device can be used in combination with various other materials present in the device. The combinations of these materials are described in more detail in U.S. Pat. App. No. 20160359122 at paragraphs 0132-0161, which is incorporated by reference herein in its entirety. The materials described or referred to the disclosure are non-limiting examples of materials that may be useful in combination with the compounds disclosed herein, and one of skill in the art can readily consult the literature to identify other materials that may be useful in combination.

The materials described herein as useful for a particular layer in an organic light-emitting device may be used in combination with a variety of other materials present in the device. For example, dopants disclosed herein may be used in combination with a wide variety of hosts, transport layers, blocking layers, injection layers, electrodes and other layers that may be present. The combination of these materials is described in detail in paragraphs 0080-0101 of U.S. Pat. App. No. 20150349273, which is incorporated by reference herein in its entirety. The materials described or referred to the disclosure are non-limiting examples of materials that may be useful in combination with the compounds disclosed herein, and one of skill in the art can readily consult the literature to identify other materials that may be useful in combination.

In the embodiments of material synthesis, all reactions were performed under nitrogen protection unless otherwise stated. All reaction solvents were anhydrous and used as received from commercial sources. Synthetic products were structurally confirmed and tested for properties using one or more conventional equipment in the art (including, but not limited to, nuclear magnetic resonance instrument produced by BRUKER, liquid chromatograph produced by SHIMADZU, liquid chromatograph-mass spectrometry produced by SHIMADZU, gas chromatograph-mass spectrometry produced by SHIMADZU, differential Scanning calorimeters produced by SHIMADZU, fluorescence spectrophotometer produced by SHANGHAI LENGGUANG TECH., electrochemical workstation produced by WUHAN CORRTEST, and sublimation apparatus produced by ANHUI BEQ, etc.) by methods well known to the persons skilled in the art. In the embodiments of the device, the characteristics of the device were also tested using conventional equipment in the art (including, but not limited to, evaporator produced by ANGSTROM ENGINEERING, optical testing system produced by SUZHOU FATAR, life testing system produced by SUZHOU FATAR, and ellipsometer produced by BEIJING ELLITOP, etc.) by methods well known to the persons skilled in the art. As the persons skilled in the art are aware of the above-mentioned equipment use, test methods and other related contents, the inherent data of the sample can be obtained with certainty and without influence, so the above related contents are not further described in this patent.

### Synthesis Example 1: Synthesis of Metal Complex 43

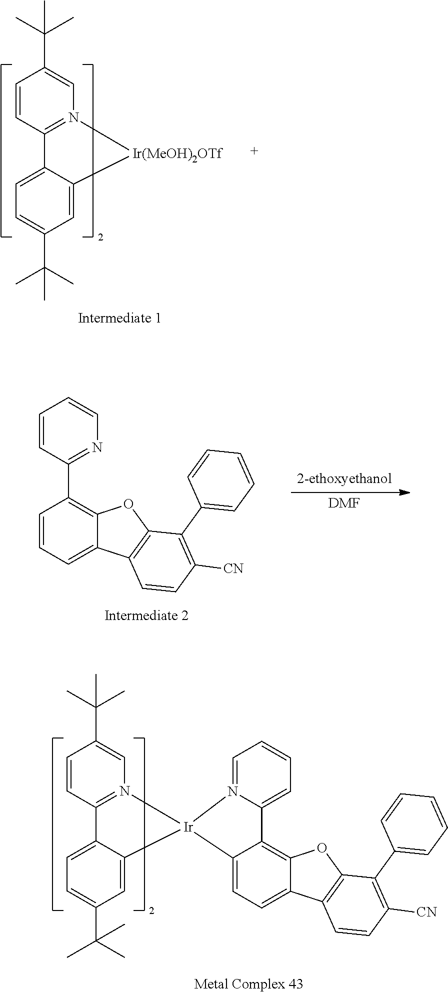

Intermediate 1 (2.9 g, 3.1 mmol), Intermediate 2 (1.5 g, 4.3 mmol), 2-ethoxyethanol (30 mL) and DMF (30 mL) were sequentially added into a dry 250 mL round-bottom flask and heated to react for 144 h at 90° C. under N₂ protection. The solution was cooled, filtered through Celite, and washed twice with dichloromethane. The organic phases were collected, concentrated under reduced pressure, and purified through column chromatography to obtain Metal Complex 43 as a yellow solid (2.0 g, 1.9 mmol with a yield of 60%). The product was confirmed as the target product with a molecular weight of 1070.4.

### Synthesis Example 2: Synthesis of Metal Complex 91

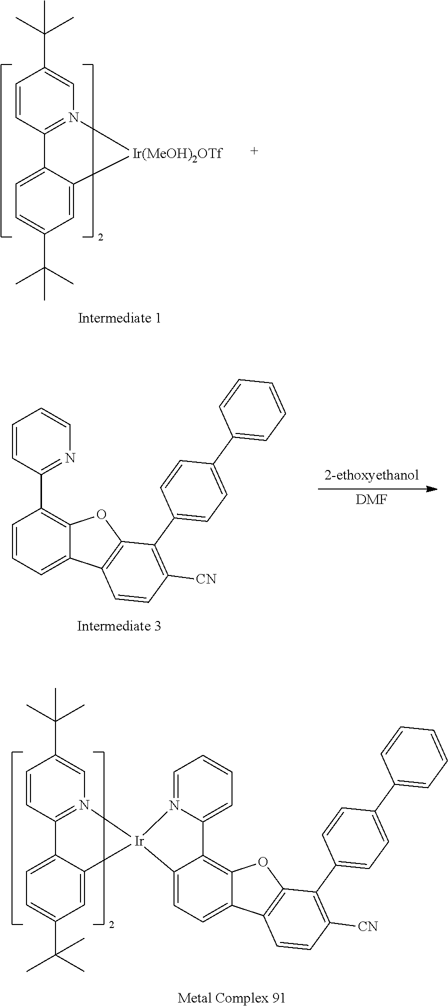

Intermediate 3 (1.1 g, 2.5 mmol), Intermediate 1 (1.7 g, 1.8 mmol), 2-ethoxyethanol (30 mL) and DMF (30 mL) were sequentially added into a dry 250 mL round-bottom flask and heated to react for 120 h at 100° C. under N₂ protection. The solution was cooled, filtered through Celite, and washed twice with dichloromethane. The organic phases were collected, concentrated under reduced pressure, and purified through column chromatography to obtain Metal Complex 91 as a yellow solid (0.95 g, 0.8 mmol with a yield of 46%). The product was confirmed as the target product with a molecular weight of 1146.5.

### Synthesis Example 3: Synthesis of Metal Complex 227

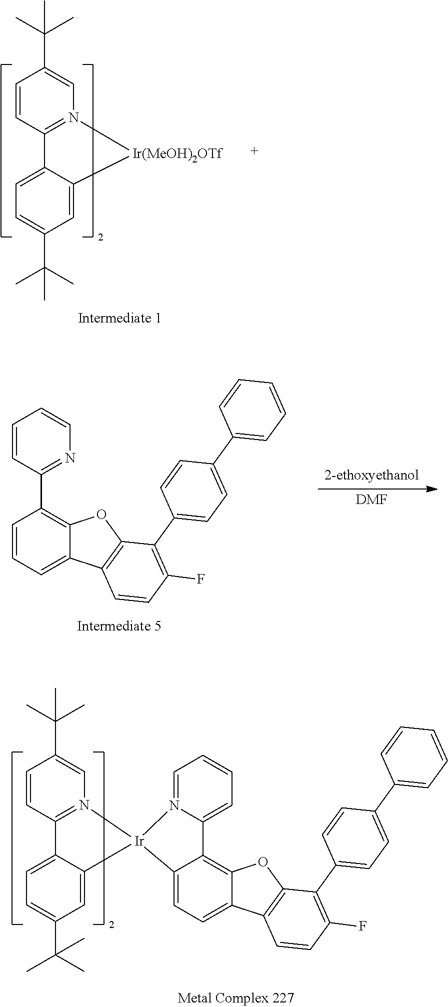

Intermediate 5 (0.75 g, 1.8 mmol), Intermediate 1 (0.9 g, 0.95 mmol), 2-ethoxyethanol (30 mL) and DMF (30 mL) were sequentially added into a dry 250 mL round-bottom flask and heated to react for 120 h at 100° C. under N₂ protection. The solution was cooled, filtered through Celite, and washed twice with dichloromethane. The organic phases were collected, concentrated under reduced pressure, and purified through column chromatography to obtain Metal Complex 227 as a yellow solid (0.34 g, 0.2 mmol with a yield of 31%). The product was confirmed as the target product with a molecular weight of 1139.5.

### Synthesis Example 4: Synthesis of Metal Complex 275

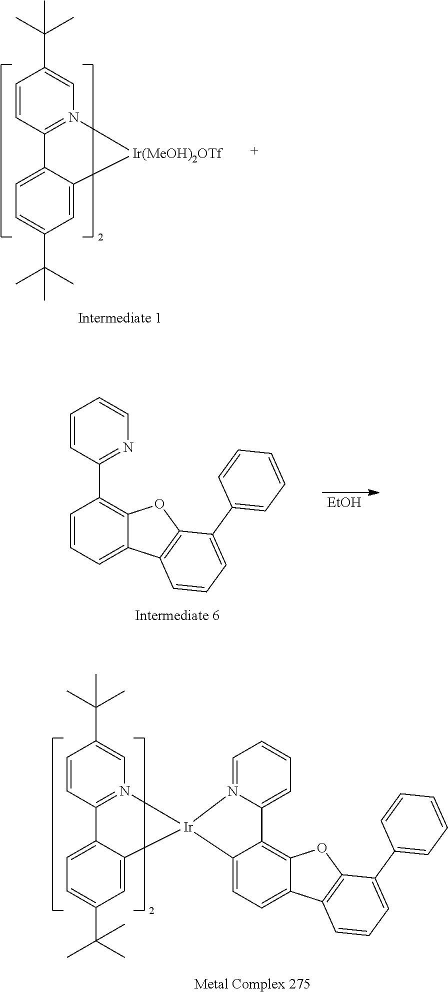

Intermediate 6 (0.45 g, 0.14 mmol), Intermediate 1 (0.88 g, 0.94 mmol) and ethanol (60 mL) were sequentially added into a dry 250 mL round-bottom flask and heated to reflux to react for 48 h under N₂ protection. The reaction was cooled, filtered through Celite, and washed twice with methanol and n-hexane separately. Yellow solids on the Celite were dissolved with dichloromethane. The organic phases were collected, concentrated under reduced pressure, and purified through column chromatography to obtain Metal Complex 275 as a yellow solid (0.13 g, 0.1 mmol with a yield of 13.2%). The product was confirmed as the target product with a molecular weight of 1045.4.

### Synthesis Example 5: Synthesis of Metal Complex 297

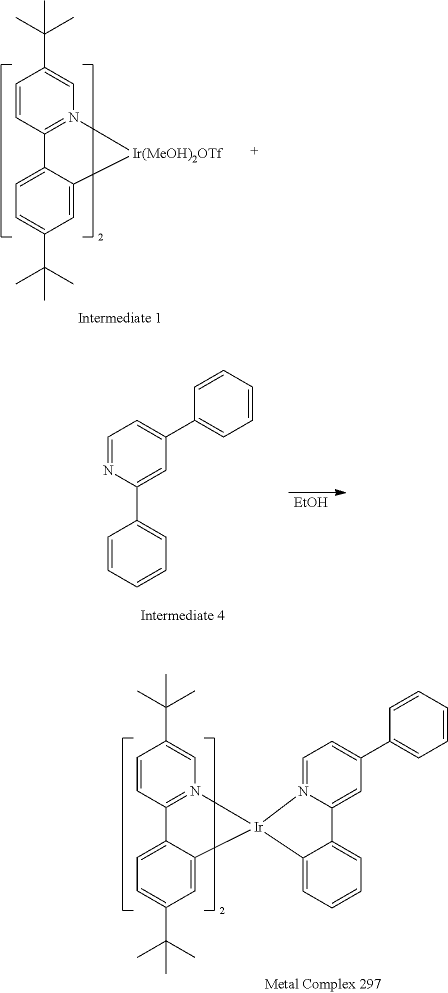

Intermediate 4 (1.0 g, 4.3 mmol), Intermediate 1 (3.1 g, 3.3 mmol), 2-ethoxyethanol (30 mL) and DMF (30 mL) were sequentially added into a dry 250 mL round-bottom flask and heated to reflux to react for 120 h under N₂ protection. The reaction was cooled, filtered through Celite, and washed twice with methanol and n-hexane separately. Yellow solids on the Celite were dissolved with dichloromethane. The organic phases were collected, concentrated under reduced pressure, and purified through column chromatography to obtain Metal Complex 297 as a yellow solid (0.41 g, 0.43 mmol with a yield of 13%). The product was confirmed as the target product with a molecular weight of 955.4.

Those skilled in the art will appreciate that the above preparation methods are merely exemplary. Those skilled in the art can obtain other compound structures of the present disclosure through the modifications of the preparation methods.

### Device Example 1

First, a glass substrate having an indium tin oxide (ITO) anode with a thickness of 80 nm was cleaned and then treated with oxygen plasma and UV ozone. After the treatment, the substrate was dried in a glovebox to remove moisture. Then, the substrate was mounted on a substrate holder and placed in a vacuum chamber. Organic layers specified below were sequentially deposited through vacuum thermal evaporation on the ITO anode at a rate of 0.2 to 2 Angstroms per second and a vacuum degree of about 10⁻⁸ torr. Compound HI was used as a hole injection layer (HIL). Compound HT was used as a hole transporting layer (HTL). Compound H1 was used as an electron blocking layer (EBL). Metal Complex 43 of the present disclosure was doped in Compound H1 and Compound H2 as a dopant, and the resulting mixture was deposited for use as an emissive layer (EML). On the EML, Compound HB was deposited as a hole blocking layer (HBL). On the HBL, Compound ET and 8-hydroxyquinolinolato-lithium (Liq) were co-deposited for use as an electron transporting layer (ETL). Finally, 8-hydroxyquinolinolato-lithium (Liq) was deposited as an electron injection layer with a thickness of 1 nm and Al was deposited as a cathode with a thickness of 120 nm. The device was transferred back to the glovebox and encapsulated with a glass lid to complete the device.

### Device Comparative Example 1

The implementation mode in Device Comparative Example 1 was the same as that in Device Example 1, except that in the EML, Metal Complex 43 of the present disclosure was replaced with Compound GD1.

### Device Comparative Example 2

The implementation mode in Device Comparative Example 2 was the same as that in Device Example 1, except that in the EML, Metal Complex 43 of the present disclosure was replaced with Compound GD2.

Detailed structures and thicknesses of layers of the devices are shown in the following table. A layer using more than one material is obtained by doping different compounds at their weight ratio as recorded.

TABLE 1
Part of device structures in Example 1 and Comparative Examples 1 and 2

| Device ID | HIL | HTL | EBL | EML | HBL | ETL |
|---|---|---|---|---|---|---|
| Example 1 | Compound HI | Compound HT | Compound H1 | Compound | Compound HB | Compound |
| | (100 Å) | (350 Å) | (50 Å) | H1:Compound | (50 Å) | ET:Liq |
| | | | | H2:Metal Complex 43 | | (40:60) |
| | | | | (46:46:8) (400 Å) | | (350 Å) |
| Comparative | Compound HI | Compound HT | Compound H1 | Compound | Compound HB | Compound |
| Example 1 | (100 Å) | (350 Å) | (50 Å) | H1:Compound | (50 Å) | ET:Liq |
| | | | | H2:Compound GD1 | | (40:60) |
| | | | | (46:46:8) (400 Å) | | (350 Å) |
| Comparative | Compound HI | Compound HT | Compound H1 | Compound | Compound HB | Compound |
| Example 2 | (100 Å) | (350 Å) | (50 Å) | H1:Compound | (50 Å) | ET:Liq |
| | | | | H2:Compound GD2 | | (40:60) |
| | | | | (46:46:8) (400 Å) | | (350 Å) |

The structures of the materials used in the devices are shown as follows:

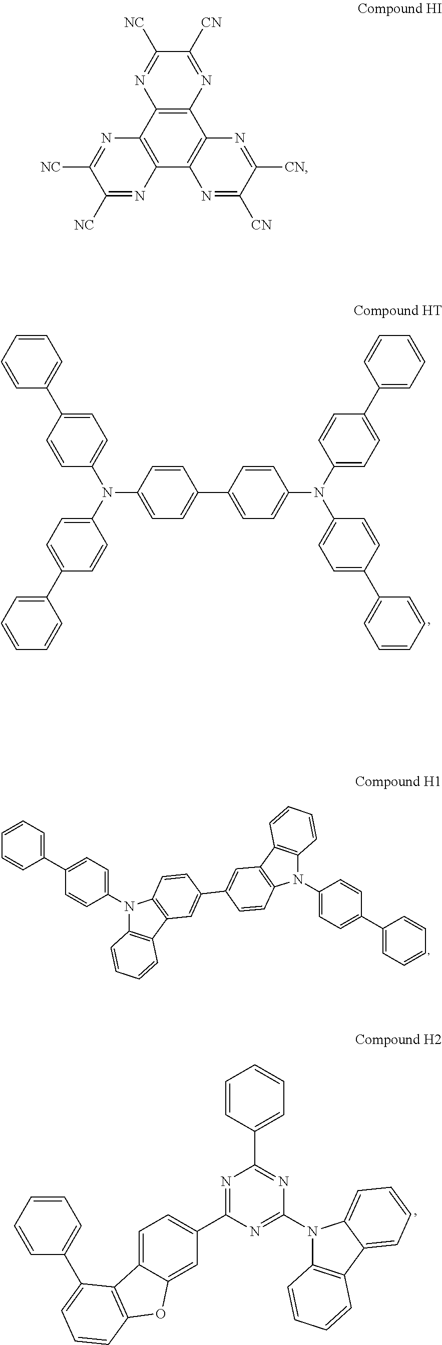 [SMILES: N#Cc1nc2c3nc(C#N)c(C#N)nc3c3nc(C#N)c(C#N)nc3c2nc1C#N ; c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1 ; c1ccc(-c2ccc(-n3c4ccccc4c4cc(-c5ccc6c(c5)c5ccccc5n6-c5ccc(-c6ccccc6)cc5)ccc43)cc2)cc1 ; c1ccc(-c2nc(-c3ccc4c(c3)oc3cccc(-c5ccccc5)c34)nc(-n3c4ccccc4c4ccccc43)n2)cc1]

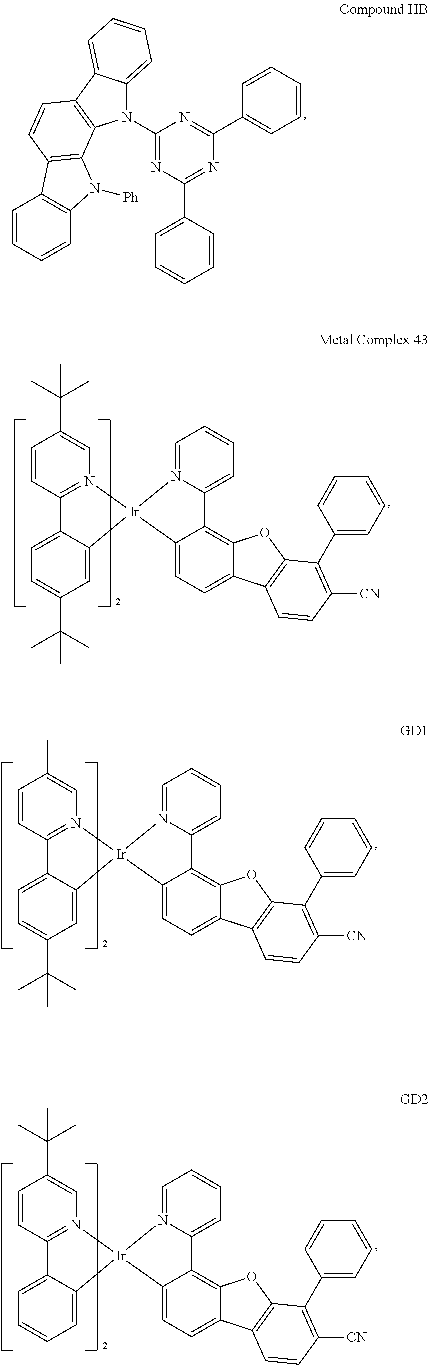

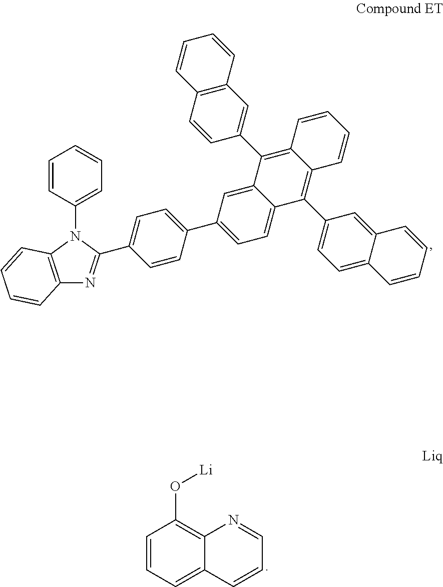

Current-voltage-luminance (IVL) characteristics of the devices were measured. The CIE data, maximum emission wavelength λₘₐₓ, current efficiency (CE) and external quantum efficiency (EQE) of each device were measured at 1000 cd/m². The evaporation temperature (T_{Sub}) of a material is the temperature tested when the metal complex is subjected to vacuum thermal evaporation at a rate of 0.2 angstroms per second and a vacuum degree of about 10⁻⁸ Torr. Lifetime (LT97) data was tested at a constant current of 80 mA/cm². The data was recorded and shown in Table 2.

TABLE 2
Relevant data in Example 1 and Comparative Examples 1 and 2

| | T_{Sub} | CIE | λₘₐₓ | CE | EQE | LT97 |
|---|---|---|---|---|---|---|
| Device ID | (° C.) | (x, y) | (nm) | (cd/A) | (%) | (h) |
| Example 1 | 241 | (0.363, 0.619) | 535 | 101 | 25.62 | 50.53 |
| Comparative | 250 | (0.355, 0.625) | 534 | 98 | 24.77 | 18.51 |
| Example 1 | | | | | | |
| Comparative | 263 | (0.346, 0.632) | 531 | 100 | 25.38 | 36.01 |
| Example 2 | | | | | | |

As can be seen from the data in Table 2, the metal complexes used in Example 1, Comparative Example 1 and Comparative Example 2 all have the same ligand L_{b}; the metal complex in Example 1 differs from that in Comparative Example 1 merely in whether a substituent having four or more carbon atoms is present at a particular position of a six-membered heteroaromatic ring of the ligand Lₐ; and the metal complex in Example 1 differs from that in Comparative Example 2 merely in whether a substituent having the structure of Formula 2 is present at a particular position of an aromatic ring of the ligand Lₐ. Both the CE and EQE in Example 1 are superior to those in Comparative Example 1 and those in Comparative Example 2. In addition, it is particularly apparent that an increase in the lifetime in Example 1 is as high as 172% and 40.3% compared to that in Comparative Example 1 and that in Comparative Example 2, respectively, which is difficult for those skilled in the art to predict, indicating that a device with better performance may be obtained with the technical solution provided by the present disclosure. Moreover, Example 1 has an unexpectedly lower evaporation temperature than Comparative Example 1 and Comparative Example 2. The lower evaporation temperature indicates that the complex of the present disclosure has higher thermal stability in a process of preparing the device, which is conducive to industrial application of the material and can reduce energy consumption in industrialization.

### Device Example 2

The implementation mode in Device Example 2 was the same as that in Device Example 1, except that in the EML, Metal Complex 43 of the present disclosure was replaced with Metal Complex 91.

### Device Comparative Example 3

The implementation mode in Device Comparative Example 3 was the same as that in Device Example 1, except that in the EML, Metal Complex 43 of the present disclosure was replaced with Compound GD3.

### Device Comparative Example 4

The implementation mode in Device Comparative Example 4 was the same as that in Device Example 1, except that in the EML, Metal Complex 43 of the present disclosure was replaced with Compound GD4.

### Device Comparative Example 5

The implementation mode in Device Comparative Example 5 was the same as that in Device Example 1, except that in the EML, Metal Complex 43 of the present disclosure was replaced with Compound GD5.

Detailed structures and thicknesses of layers of the devices are shown in the following table. A layer using more than one material is obtained by doping different compounds at their weight ratio as recorded.

TABLE 3
Device structures in Example 2 and Comparative Examples 3 to 5

| Device ID | HIL | HTL | EBL | EML | HBL | ETL |
|---|---|---|---|---|---|---|
| Example 2 | Compound HI | Compound HT | Compound H1 | Compound | Compound HB | Compound |
| | (100 Å) | (350 Å) | (50 Å) | H1:Compound | (50 Å) | ET:Liq |
| | | | | H2:Metal Complex 91 | | (40:60) |
| | | | | (46:46:8) (400 Å) | | (350 Å) |
| Comparative | Compound HI | Compound HT | Compound H1 | Compound | Compound HB | Compound |
| Example 3 | (100 Å) | (350 Å) | (50 Å) | H1:Compound | (50 Å) | ET:Liq |
| | | | | H2:Compound GD3 | | (40:60) |
| | | | | (46:46:8) (400 Å) | | (350 Å) |
| Comparative | Compound HI | Compound HT | Compound H1 | Compound | Compound HB | Compound |
| Example 4 | (100 Å) | (350 Å) | (50 Å) | H1:Compound | (50 Å) | ET:Liq |
| | | | | H2:Compound GD4 | | (40:60) |
| | | | | (46:46:8) (400 Å) | | (350 Å) |
| Comparative | Compound HI | Compound HT | Compound H1 | Compound | Compound HB | Compound |
| Example 5 | (100 Å) | (350 Å) | (50 Å) | H1:Compound | (50 Å) | ET:Liq |
| | | | | H2:Compound GD5 | | (40:60) |
| | | | | (46:46:8) (400 Å) | | (350 Å) |

The structures of the new materials used in the devices are shown as follows:

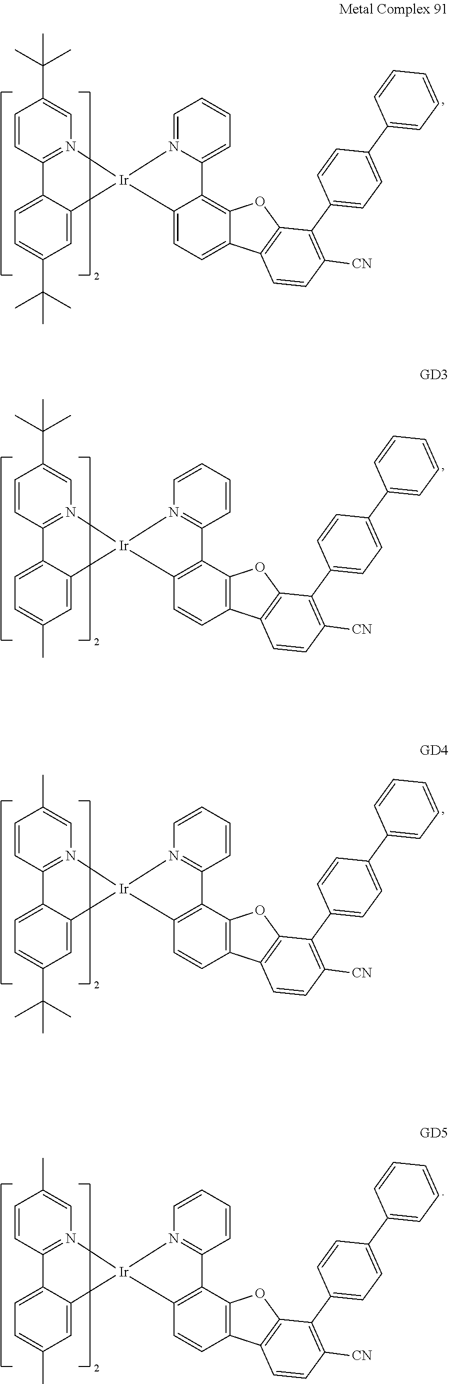

IVL characteristics of the devices were measured. The CIE data, maximum emission wavelength λₘₐₓ, FWHM, CE and EQE of each device were measured at 15 mA/cm². The evaporation temperature (T_{Sub}) of a material is the temperature tested when the metal complex is subjected to vacuum thermal evaporation at a rate of 0.2 angstroms per second and a vacuum degree of about 10⁻⁸ Torr. Lifetime (LT97) data was tested at a constant current of 80 mA/cm². The data was recorded and shown in Table 4.

TABLE 4
Relevant data in Example 2 and Comparative Examples 3 to 5

| | T_{Sub} | CIE | λₘₐₓ | FWHM | CE | EQE | LT97 |
|---|---|---|---|---|---|---|---|
| Device ID | (° C.) | (x, y) | (nm) | (nm) | (cd/A) | (%) | (h) |
| Example 2 | 273 | (0.359, 0.623) | 535 | 36.5 | 98 | 24.89 | 44.52 |
| Comparative | 290 | (0.359, 0.623) | 534 | 38.8 | 99 | 25.08 | 30.83 |
| Example 3 | | | | | | | |
| Comparative | 280 | (0.355, 0.625) | 534 | 42.0 | 97 | 24.78 | 25.01 |
| Example 4 | | | | | | | |
| Comparative | 308 | (0.352, 0.627) | 533 | 38.9 | 100 | 25.46 | 23.85 |
| Example 5 | | | | | | | |

As can be seen from the data in Table 4, the metal complexes used in Example 2 and Comparative Examples 3 to 5 all have the same ligand L_{b}, and the metal complex in Example 2 differs from that in Comparative Examples 3 to 5 merely in substituent at a particular position of the ligand Lₐ. In the case where the EQE in Example 2 and Comparative Examples 3 to 5 reaches a relatively high level, the lifetime in Example 2 is significantly improved by 44.4%, 78% and 86.7% compared to that in Comparative Examples 3 to 5, respectively, which is difficult for those skilled in the art to predict; Example 2 has a narrower FWHM, which is narrowed by 2.3 nm, 5.5 nm and 2.4 nm, respectively; moreover, Example 2 has a lower evaporation temperature, which is unexpectedly lowered by 17° C., 7° C. and 35° C., respectively. This further indicates that the device with better performance may be obtained with the technical solution provided by the present disclosure and that the complex of the present disclosure has higher thermal stability in the process of preparing the device, which is more conducive to the industrial application of the material and can reduce the energy consumption in the industrialization.

### Device Example 3

The implementation mode in Device Example 3 was the same as that in Device Example 1, except that in the EML, Metal Complex 43 of the present disclosure was replaced with Metal Complex 275, and in the EML, the ratio of Compound H1, Compound H2 and Metal Complex 275 was 47:47:6.

### Device Example 4

The implementation mode in Device Example 4 was the same as that in Device Example 1, except that in the EML, Metal Complex 43 of the present disclosure was replaced with Metal Complex 227.

### Device Comparative Example 6

The implementation mode in Device Comparative Example 6 was the same as that in Device Example 3, except that in the EML, Metal Complex 275 of the present disclosure was replaced with Compound GD6.

### Device Comparative Example 7

The implementation mode in Device Comparative Example 7 was the same as that in Device Example 1, except that in the EML, Metal Complex 43 of the present disclosure was replaced with Compound GD7.

### Device Comparative Example 8

The implementation mode in Device Comparative Example 8 was the same as that in Device Example 1, except that in the EML, Metal Complex 43 of the present disclosure was replaced with Compound GD8.

Detailed structures and thicknesses of layers of the devices are shown in the following table. A layer using more than one material is obtained by doping different compounds at their weight ratio as recorded.

TABLE 5
Device structures in Examples 3 and 4 and Comparative Examples 6 to 8

| Device ID | HIL | HTL | EBL | EML | HBL | ETL |
|---|---|---|---|---|---|---|
| Example 3 | Compound HI | Compound HT | Compound H1 | Compound | Compound HB | Compound |
| | (100 Å) | (350 Å) | (50 Å) | H1:Compound | (50 Å) | ET:Liq |
| | | | | H2:Metal Complex 275 | | (40:60) |
| | | | | (47:47:6) (400 Å) | | (350 Å) |
| Example 4 | Compound HI | Compound HT | Compound H1 | Compound | Compound HB | Compound |
| | (100 Å) | (350 Å) | (50 Å) | H1:Compound | (50 Å) | ET:Liq |
| | | | | H2:Metal Complex 227 | | (40:60) |
| | | | | (46:46:8) (400 Å) | | (350 Å) |
| Comparative | Compound HI | Compound HT | Compound H1 | Compound | Compound HB | Compound |
| Example 6 | (100 Å) | (350 Å) | (50 Å) | H1:Compound | (50 Å) | ET:Liq |
| | | | | H2:Compound GD6 | | (40:60) |
| | | | | (47:47:6) (400 Å) | | (350 Å) |
| Comparative | Compound HI | Compound HT | Compound H1 | Compound | Compound HB | Compound |
| Example 7 | (100 Å) | (350 Å) | (50 Å) | H1:Compound | (50 Å) | ET:Liq |
| | | | | H2:Compound GD7 | | (40:60) |
| | | | | (46:46:8) (400 Å) | | (350 Å) |
| Comparative | Compound HI | Compound HT | Compound H1 | Compound | Compound HB | Compound |
| Example 8 | (100 Å) | (350 Å) | (50 Å) | H1:Compound | (50 Å) | ET:Liq |
| | | | | H2:Compound GD8 | | (40:60) |
| | | | | (46:46:8) (400 Å) | | (350 Å) |

The structures of the new materials used in the devices are shown as follows:

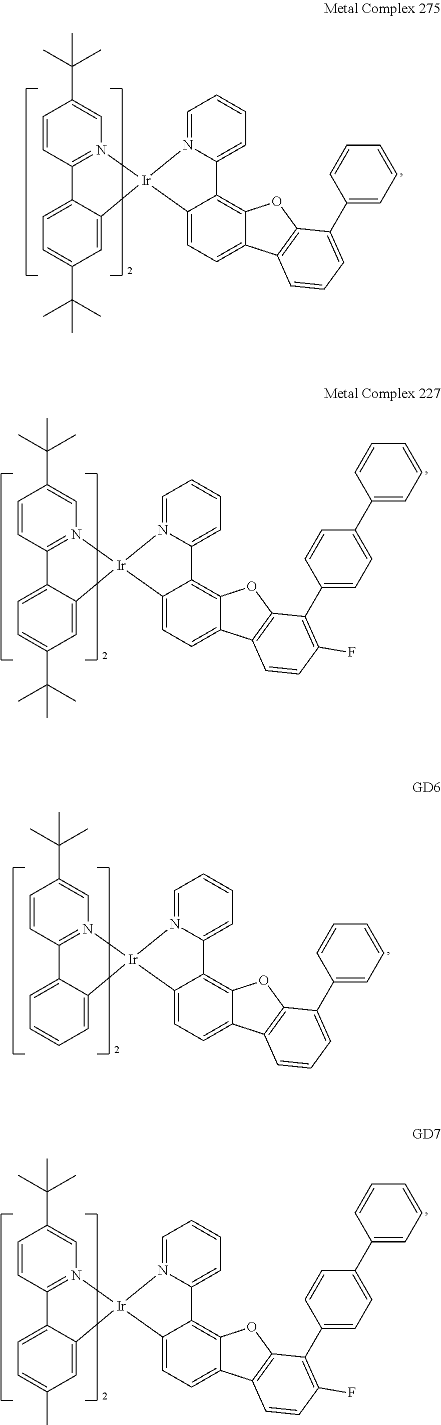

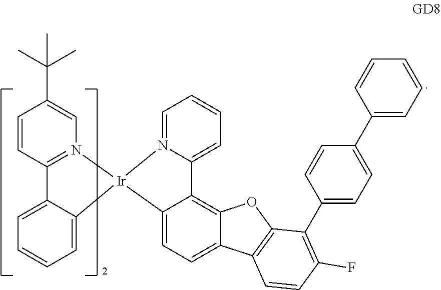

IVL characteristics of the devices were measured. The CIE data, maximum emission wavelength λₘₐₓ, FWHM, CE and EQE of each device were measured at 10 mA/cm². The evaporation temperature (T_{Sub}) of a material is the temperature tested when the metal complex is subjected to vacuum thermal evaporation at a rate of 0.2 angstroms per second and a vacuum degree of about 10⁻⁸ Torr. The data was recorded and shown in Table 6.

TABLE 6
Relevant data in Examples 3 and
4 and Comparative Examples 6 to 8

| | T_{Sub} | CIE | λₘₐₓ | FWHM | CE | EQE |
|---|---|---|---|---|---|---|
| Device ID | (° C.) | (x, y) | (nm) | (nm) | (cd/A) | (%) |
| Example 3 | 249 | (0.365, 0.614) | 533 | 60.0 | 85 | 22.20 |
| Comparative | 258 | (0.352, 0.623) | 531 | 58.8 | 82 | 21.35 |
| Example 6 | | | | | | |
| Example 4 | 248 | (0.374, 0.608) | 534 | 60.5 | 94 | 24.58 |
| Comparative | 261 | (0.375, 0.607) | 534 | 61.6 | 90 | 23.51 |
| Example 7 | | | | | | |
| Comparative | 292 | (0.360, 0.618) | 531 | 59.8 | 87 | 22.59 |
| Example 8 | | | | | | |

As can be seen from the data in Table 6, the metal complexes of the present disclosure used in Example 3 and Comparative Example 6 both have the same ligand L_{b}, and the metal complex in Example 3 differs from that in Comparative Example 6 merely in whether the substituent represented by Formula 2 is present at the particular position of the ligand Lₐ. The CE and EQE in Example 3 are improved by 3.7% and 4.0% compared to those in Comparative Example 6, respectively. Moreover, the evaporation temperature in Example 3 is lowered by 9° C. compared to that in Comparative Example 6.

Similarly, Example 4 differs from Comparative Examples 7 and 8 merely in whether the substituent represented by Formula 2 is present at the particular position of the ligand Lₐ. The CE and EQE in Example 4 are improved by 4.4% and 4.6% compared to those in Comparative Example 7, respectively. The CE and EQE in Example 4 are improved by 8.0% and 8.8% compared to those in Comparative Example 8, respectively. In the case where Comparative Examples 7 and 8 have excellent performance, the performance in Example 4 is very rare compared to that in Comparative Examples 7 and 8. Moreover, Example 4 has a lower evaporation temperature than Comparative Examples 7 and 8, and the evaporation temperature is lowered by 13° C. and 44° C., respectively.

The above results indicate that the device using the metal complex of the present disclosure can obtain better device performance and that the metal complex of the present disclosure has higher thermal stability in the process of preparing the device, which is more conducive to the industrial application of the material and can reduce the energy consumption in the industrialization.

To conclude, the metal complex comprising both the ligand Lₐ having the structure of Formula 1A and the ligand L_{b} having the structure of Formula 1B in the present application can obtain very excellent device performance, and in particular, an significant increase in device lifetime which is difficult to predict. Moreover, unexpectedly, the evaporation temperature can be lowered so that the metal complex of the present disclosure has higher thermal stability in the process of preparing the device, which is more conducive to the industrial application of the material and can reduce the energy consumption in the industrialization.

It is to be understood that various embodiments described herein are merely examples and not intended to limit the scope of the present disclosure. Therefore, it is apparent to the persons skilled in the art that the present disclosure as claimed may include variations of specific embodiments and preferred embodiments described herein. Many of materials and structures described herein may be substituted with other materials and structures without departing from the spirit of the present disclosure. It is to be understood that various theories as to why the present disclosure works are not intended to be limitative.

What is claimed is:

1. A metal complex having a general formula of M(Lₐ)ₘ(L_{b})ₙ(L_{c})_{q};
wherein
Lₐ, L_{b} and L_{c} are a first ligand, a second ligand and a third ligand coordinated to the metal M, respectively, and Lₐ, L_{b} and L_{c} are the same or different; wherein Lₐ, L_{b} and L_{c} can be optionally joined to form a multidentate ligand;
the metal M is selected from a metal with a relative atomic mass greater than 40; and
m is selected from 1 or 2, n is selected from 1 or 2, q is selected from 0 or 1, and m+n+q equals an oxidation state of M; when m is 2, two Lₐ are identical or different; when n is 2, two L_{b} are identical or different;
Lₐ has, at each occurrence identically or differently, a structure represented by Formula 1A and L_{b} has, at each occurrence identically or differently, a structure represented by Formula 1B: 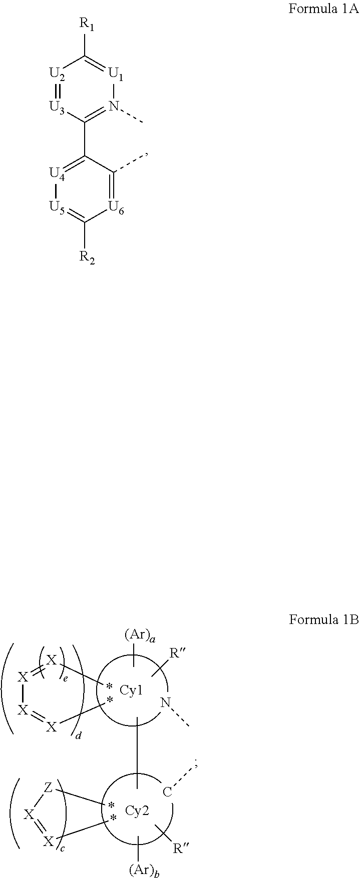
wherein
the ring Cy1 is, at each occurrence identically or differently, selected from a heteroaromatic ring having 5 to 6 ring atoms;
the ring Cy2 is, at each occurrence identically or differently, selected from a benzene ring or a heteroaromatic ring having 5 to 6 ring atoms;
Ar is, at each occurrence identically or differently, selected from substituted or unsubstituted aryl having 6 to 30 carbon atoms, substituted or unsubstituted heteroaryl having 3 to 30 carbon atoms or a combination thereof;
a, b, c, d and e are, at each occurrence identically or differently, selected from 0 or 1, and at least one of a, b, c and d is selected from 1;
Z is, at each occurrence identically or differently, selected from O or X═X;
X is, at each occurrence identically or differently, selected from N or CR′;
U₁ to U₆ are, at each occurrence identically or differently, selected from CRᵤ or N;
R″ is, at each occurrence identically or differently, selected from mono-substitution, multiple substitutions or non-substitution;
R′, R″ and Rᵤ are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group and combinations thereof;
“*” represents a position where the ring Cy1 or the ring Cy2 is joined;
in Formula 1A, adjacent substituents Rᵤ can be optionally joined to form a ring;
in Formula 1B, adjacent substituents R′ and R″ can be optionally joined to form a ring; and
R₁ is, at each occurrence identically or differently, selected from the group consisting of: substituted or unsubstituted alkyl having 4 to 20 carbon atoms, substituted or unsubstituted cycloalkyl having 4 to 20 ring carbon atoms, substituted or unsubstituted heteroalkyl having 4 to 20 carbon atoms, a substituted or unsubstituted heterocyclic group having 4 to 20 ring atoms, substituted or unsubstituted arylalkyl having 7 to 30 carbon atoms, substituted or unsubstituted alkoxy having 4 to 20 carbon atoms, substituted or unsubstituted aryloxy having 6 to 30 carbon atoms, substituted or unsubstituted alkenyl having 4 to 20 carbon atoms, substituted or unsubstituted alkynyl having 4 to 20 carbon atoms, substituted or unsubstituted aryl having 6 to 30 carbon atoms, substituted or unsubstituted heteroaryl having 4 to 30 carbon atoms and combinations thereof;
R₂ has a structure represented by Formula 2: 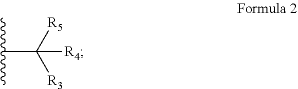
wherein
the number of carbon atoms in R₂ is greater than or equal to 4;
R₃, R₄ and R₅ are, at each occurrence identically or differently, selected from the group consisting of: halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group and combinations thereof; 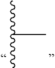
represents a position where Formula 2 is joined to Formula 1A;
two adjacent substituents of R₃, R₄ and R₅ can be optionally joined to form a ring; and
L_{c} is a monoanionic bidentate ligand.

2. The metal complex according to claim 1, wherein the ring Cy1 is, at each occurrence identically or differently, selected from any structure of the group consisting of the following: 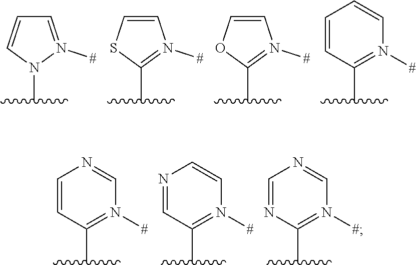
and/or
the ring Cy2 is, at each occurrence identically or differently, selected from any structure of the group consisting of the following: 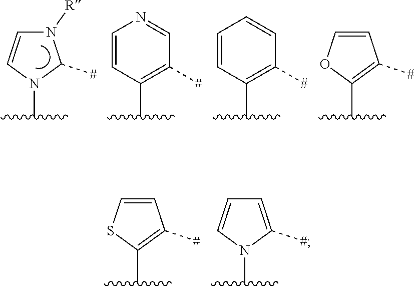
wherein “#” represents a position where the ring Cy1/Cy2 is joined to the Metal M, and 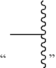
represents a position where the ring Cy1 is joined to the ring Cy2; and
definitions of substituents R″ and Ar on the ring Cy1 and that the structure is fused at the position “*” are the same as those in claim 1, and definitions of substituents R″ and Ar on the ring Cy2 and that the structure is fused at the position “*” are the same as those in claim 1.

3. The metal complex according to claim 1, wherein L_{b} has, at each occurrence identically or differently, a structure represented by any one of Formulas 1Ba to 1Bm: 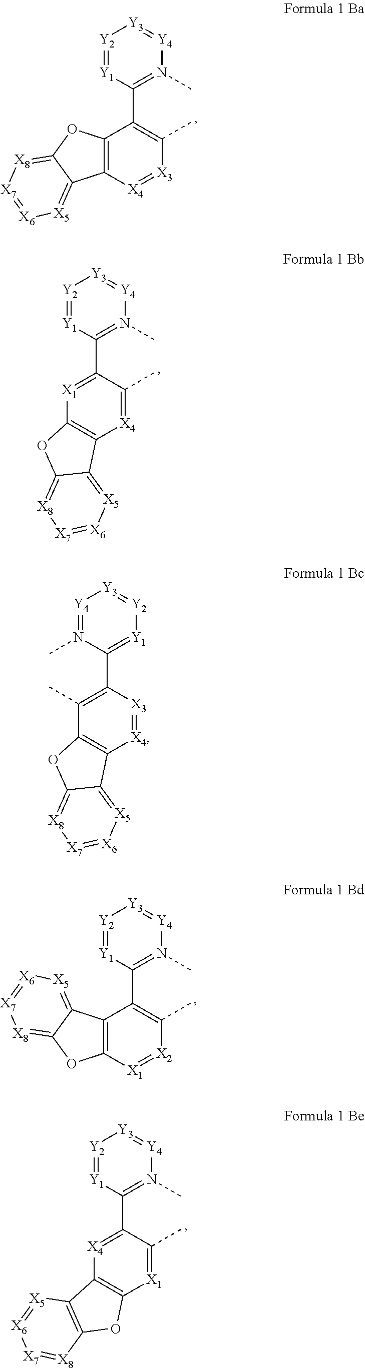 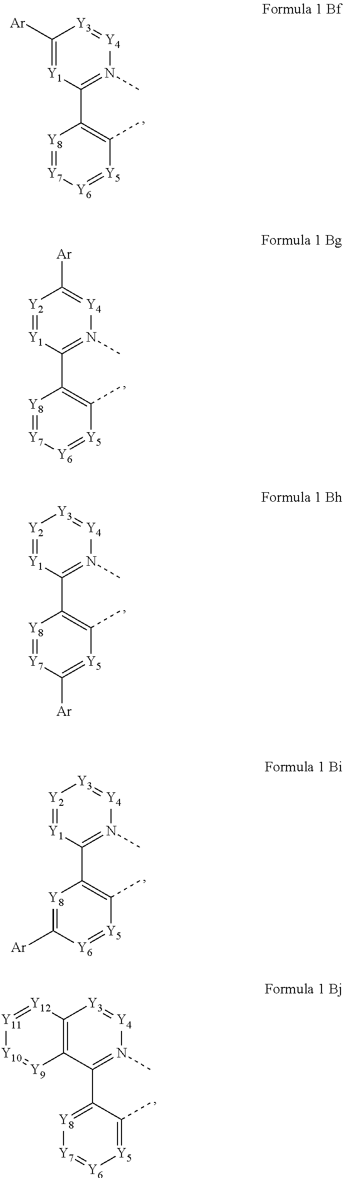 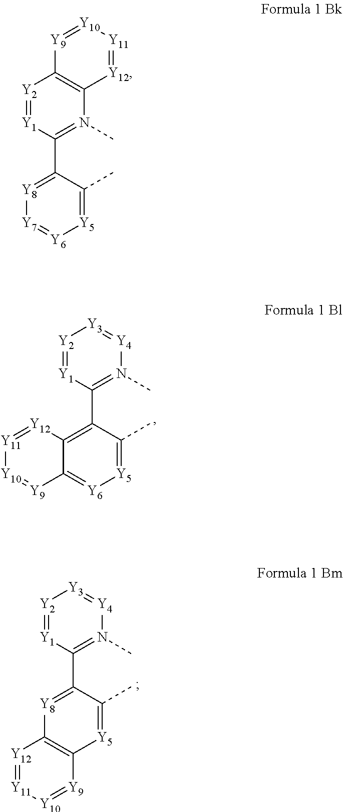
wherein
X₁ to X₈ are, at each occurrence identically or differently, selected from CRₓ or N;
Y₁ to Y₁₂ are, at each occurrence identically or differently, selected from CR_{y} or N;
Ar is, at each occurrence identically or differently, selected from substituted or unsubstituted aryl having 6 to 30 carbon atoms, substituted or unsubstituted heteroaryl having 3 to 30 carbon atoms or a combination thereof;
Rₓ and R_{y} are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group and combinations thereof;
adjacent substituents Rₓ and R_{y} can be optionally joined to form a ring; and
preferably, L_{b} has, at each occurrence identically or differently, a structure represented by any one of Formulas 1Ba to 1Bi.

4. The metal complex according to claim 1, wherein R₁ is, at each occurrence identically or differently, selected from the group consisting of: substituted or unsubstituted alkyl having 4 to 20 carbon atoms, substituted or unsubstituted cycloalkyl having 4 to 20 ring carbon atoms, substituted or unsubstituted heteroalkyl having 4 to 20 carbon atoms, a substituted or unsubstituted heterocyclic group having 4 to 20 ring atoms and combinations thereof; and
preferably, R₁ is, at each occurrence identically or differently, selected from the group consisting of: substituted or unsubstituted alkyl having 4 to 10 carbon atoms, substituted or unsubstituted cycloalkyl having 4 to 10 ring carbon atoms and combinations thereof.

5. The metal complex according to claim 1, wherein the metal M is, at each occurrence identically or differently, selected from the group consisting of Cu, Ag, Au, Ru, Rh, Pd, Os, Ir and Pt; and
preferably, M is, at each occurrence identically or differently, selected from Pt or Ir.

6. The metal complex according to claim 1, wherein R₁ has a structure represented by Formula 2.

7. The metal complex according to claim 1, wherein R₃, R₄ and R₅ are, at each occurrence identically or differently, selected from the group consisting of: halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 ring carbon atoms, a substituted or unsubstituted heterocyclic group having 3 to 20 ring atoms, substituted or unsubstituted arylalkyl having 7 to 30 carbon atoms, substituted or unsubstituted alkenyl having 2 to 20 carbon atoms, substituted or unsubstituted aryl having 6 to 30 carbon atoms or substituted or unsubstituted heteroaryl having 3 to 30 carbon atoms;
preferably, R₃, R₄ and R₅ are, at each occurrence identically or differently, selected from the group consisting of: halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms and combinations thereof; and
more preferably, R₃, R₄ and R₅ are, at each occurrence identically or differently, selected from the group consisting of: halogen, substituted or unsubstituted alkyl having 1 to 6 carbon atoms, substituted or unsubstituted cycloalkyl having 3 to 6 ring carbon atoms and combinations thereof.

8. The metal complex according to claim 1, wherein the structure represented by Formula 2 represents, at each occurrence identically or differently, any one of the following structures: 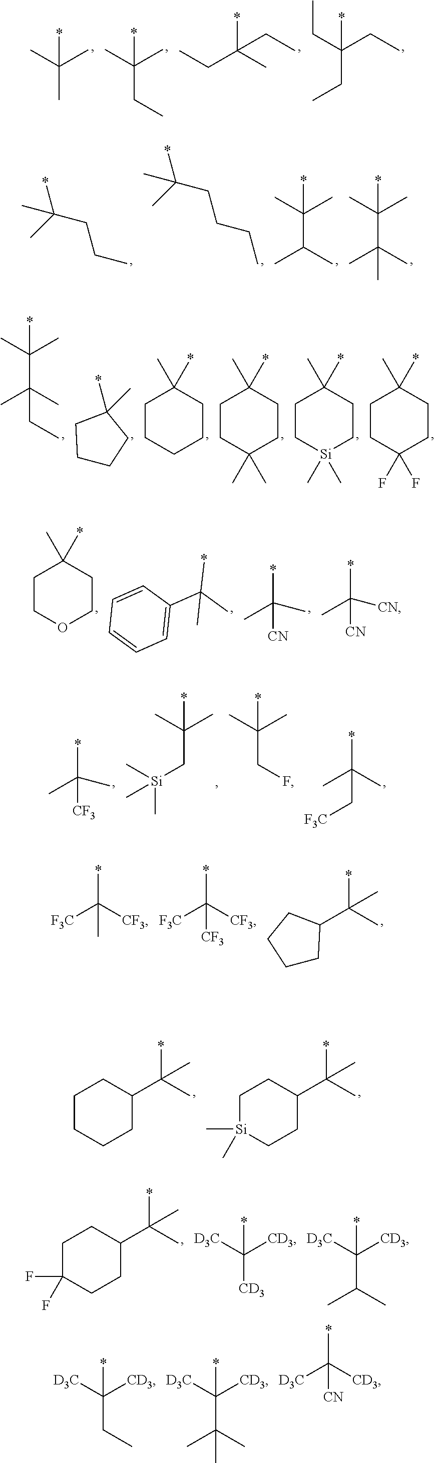 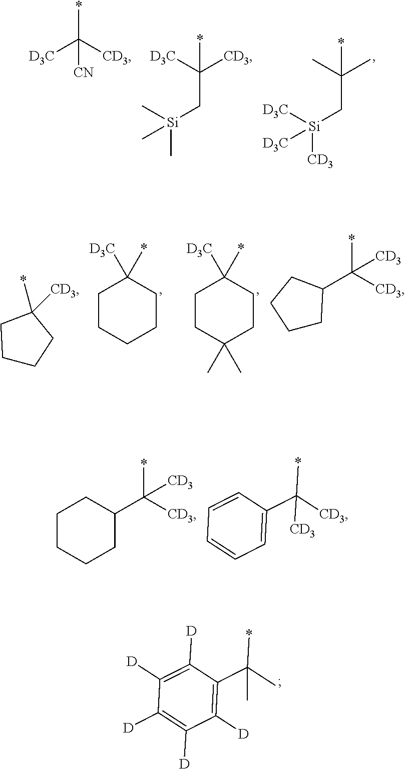
wherein “*” represents a position where the structure is joined to Formula 1A; and
optionally, hydrogen atoms in the above structures can be partially or fully substituted with deuterium atoms.

9. The metal complex according to claim 1, wherein the metal complex has a structure of a general formula of Ir(Lₐ)ₘ(L_{b})₃₋ₘ which is represented by Formula 3, Formula 4 or Formula 5: 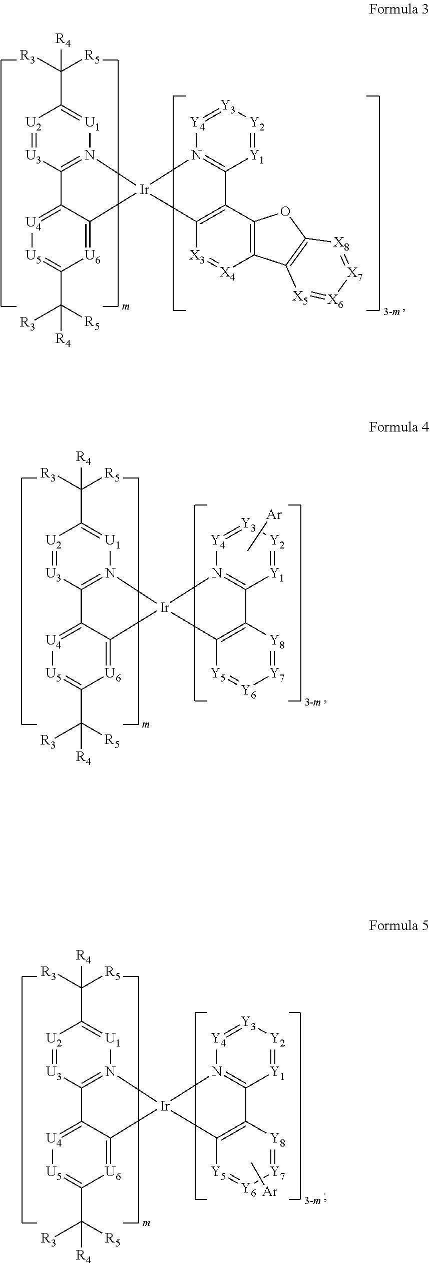
wherein
m is selected from 1 or 2; when m=1, two L_{b} are identical or different; when m=2, two Lₐ are identical or different;
X₃ to X₈ are, at each occurrence identically or differently, selected from CRₓ or N;
Y₁ to Y₈ are, at each occurrence identically or differently, selected from C, CR_{y} or N;
U₁ to U₆ are, at each occurrence identically or differently, selected from CRᵤ or N;
Ar is, at each occurrence identically or differently, selected from substituted or unsubstituted aryl having 6 to 30 carbon atoms, substituted or unsubstituted heteroaryl having 3 to 30 carbon atoms or a combination thereof;
Rₓ, R_{y} and Rᵤ are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group and combinations thereof;
R₃, R₄ and R₅ are, at each occurrence identically or differently, selected from the group consisting of: substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkenyl having 2 to 20 carbon atoms, substituted or unsubstituted aryl having 6 to 30 carbon atoms or substituted or unsubstituted heteroaryl having 3 to 30 carbon atoms;
adjacent substituents Rₓ and R_{y} can be optionally joined to form a ring; and
two adjacent substituents of R₃, R₄ and R₅ can be optionally joined to form a ring.

10. The metal complex according to claim 3, wherein Rₓ is, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted amino having 0 to 20 carbon atoms, a cyano group, a hydroxyl group, a sulfanyl group and combinations thereof; and
preferably, Rₓ is, at each occurrence identically or differently, selected from the group consisting of: hydrogen, deuterium, halogen, substituted or unsubstituted alkyl having 1 to 20 carbon atoms, substituted or unsubstituted heteroalkyl having 3 to 20 ring carbon atoms, substituted or unsubstituted aryl having 6 to 30 carbon atoms, substituted or unsubstituted heteroaryl having 3 to 30 carbon atoms, a cyano group and combinations thereof.

11. The metal complex according to claim 3, wherein at least one of X₃ to X₈ is selected from CRₓ, and Rₓ is selected from cyano or fluorine;
preferably, at least one of X₅ to X₈ is selected from CRₓ, and Rₓ is selected from cyano or fluorine; and
more preferably, X₇ or X₈ is selected from CRₓ, and Rₓ is selected from cyano or fluorine.

12. The metal complex according to claim 3, wherein at least two of X₃ to X₈ are selected from CRₓ, wherein one Rₓ is selected from cyano or fluorine, and at least another one Rₓ is selected from the group consisting of: deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group and combinations thereof;
preferably, at least two of X₅ to X₈ are selected from CRₓ, wherein one Rₓ is selected from cyano or fluorine, and at least another one Rₓ is selected from the group consisting of: deuterium, halogen, substituted or unsubstituted alkyl having 1 to 20 carbon atoms, substituted or unsubstituted cycloalkyl having 3 to 20 ring carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted amino having 0 to 20 carbon atoms, a cyano group, a hydroxyl group, a sulfanyl group and combinations thereof; and
more preferably, X₇ and X₈ are selected from CRₓ, wherein one Rₓ is cyano or fluorine, and another one Rₓ is selected from the group consisting of: deuterium, halogen, substituted or unsubstituted alkyl having 1 to 20 carbon atoms, substituted or unsubstituted cycloalkyl having 3 to 20 ring carbon atoms, substituted or unsubstituted aryl having 6 to 30 carbon atoms, substituted or unsubstituted heteroaryl having 3 to 30 carbon atoms and combinations thereof.

13. The metal complex according to claim 9, wherein U₁ to U₆ are, at each occurrence identically or differently, selected from CRᵤ, and/or Y₁ to Y₄ are, at each occurrence identically or differently, selected from CR_{y}, and/or X₃ to X₈ are, at each occurrence identically or differently, selected from CRₓ.

14. The metal complex according to claim 9, wherein Rᵤ and R_{y} are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted aryl having 6 to 30 carbon atoms, substituted or unsubstituted heteroaryl having 3 to 30 carbon atoms and combinations thereof;
preferably, Rᵤ and R_{y} are, at each occurrence identically or differently, selected from the group consisting of: hydrogen, deuterium, substituted or unsubstituted alkyl having 1 to 10 carbon atoms, substituted or unsubstituted cycloalkyl having 3 to 10 ring carbon atoms, substituted or unsubstituted aryl having 6 to 10 carbon atoms and combinations thereof; and
more preferably, Rᵤ and R_{y} are, at each occurrence identically or differently, selected from the group consisting of: hydrogen, deuterium, substituted or unsubstituted alkyl having 1 to 10 carbon atoms, substituted or unsubstituted cycloalkyl having 3 to 10 ring carbon atoms and combinations thereof.

15. The metal complex according to claim 9, wherein at least one of U₁ to U₃ is selected from N, and/or at least one of U₄ to U₆ is selected from N, and/or at least one of Y₁ to Y₄ is selected from N, and/or at least one of X₃ to X₈ is selected from N.

16. The metal complex according to claim 1, wherein Ar is, at each occurrence identically or differently, selected from substituted or unsubstituted aryl having 6 to 24 carbon atoms, substituted or unsubstituted heteroaryl having 3 to 24 carbon atoms or a combination thereof;
preferably, Ar is, at each occurrence identically or differently, selected from substituted or unsubstituted aryl having 6 to 12 carbon atoms, substituted or unsubstituted heteroaryl having 3 to 12 carbon atoms or a combination thereof; and
more preferably, Ar is, at each occurrence identically or differently, selected from substituted or unsubstituted phenyl, substituted or unsubstituted biphenyl, substituted or unsubstituted pyridyl or a combination thereof.

17. The metal complex according to claim 1, wherein Lₐ is, at each occurrence identically or differently, selected from the group consisting of the following: 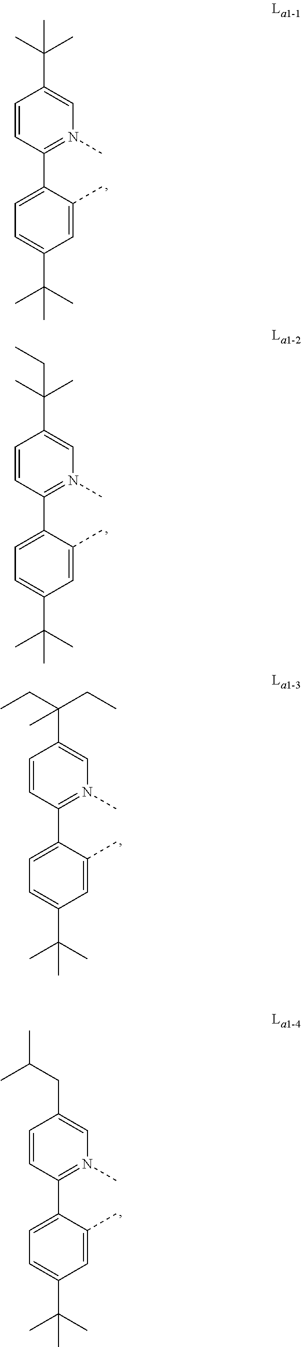 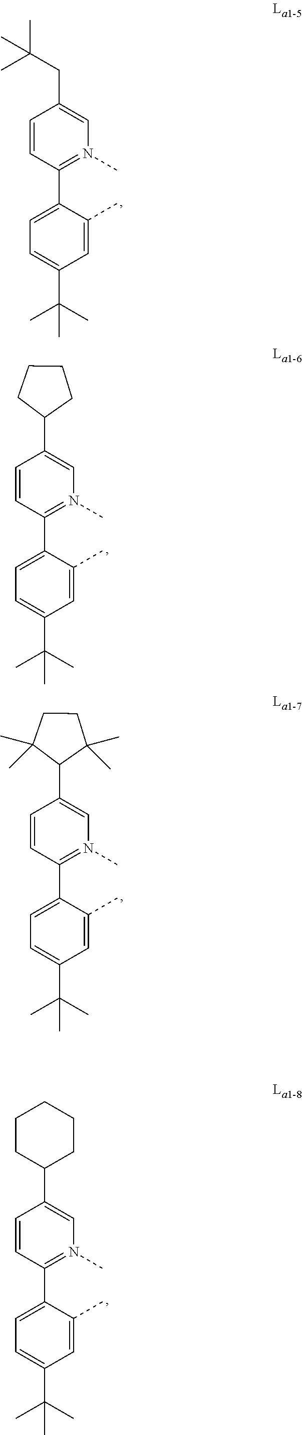 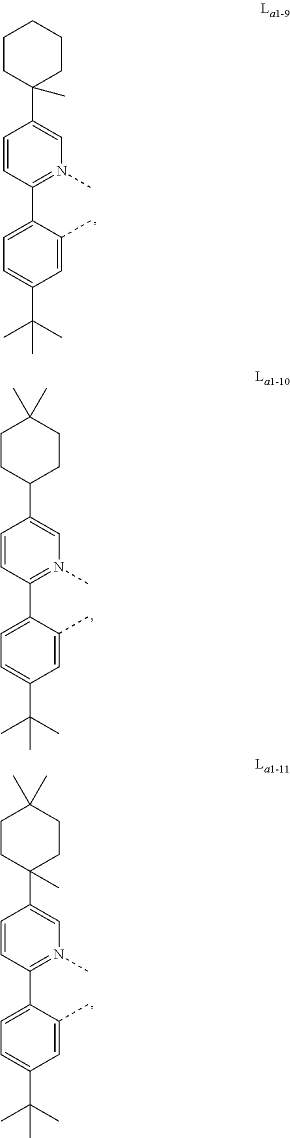 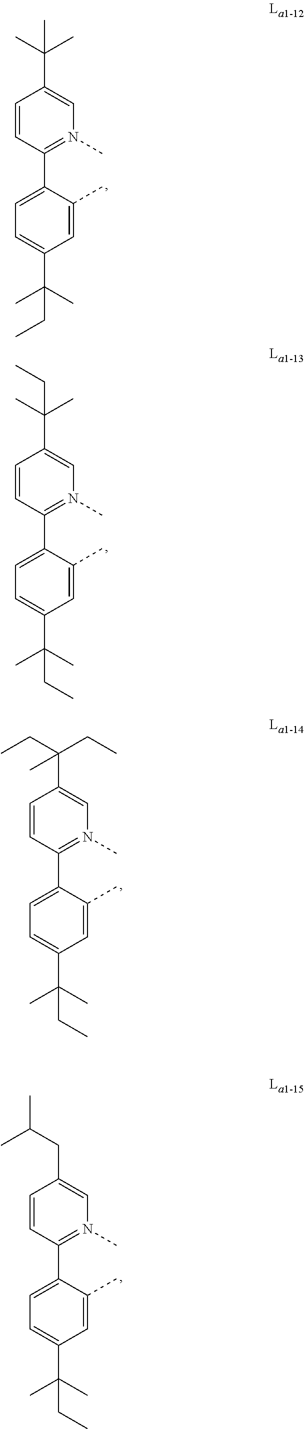 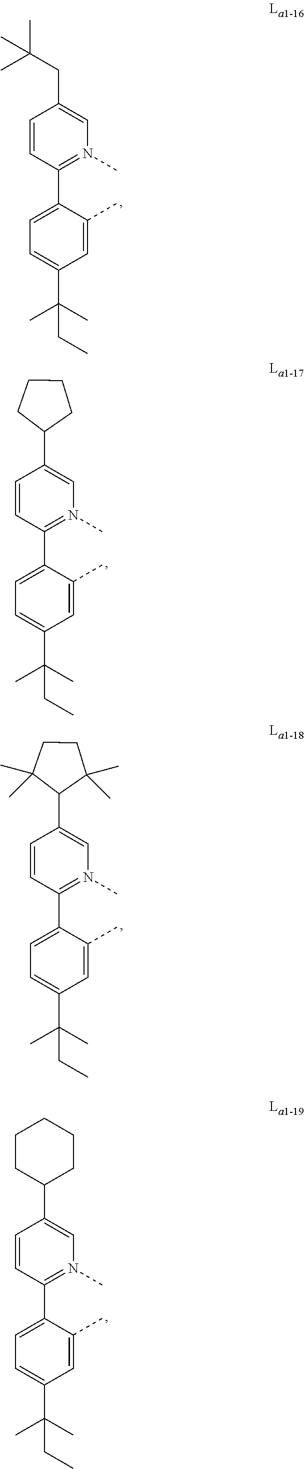 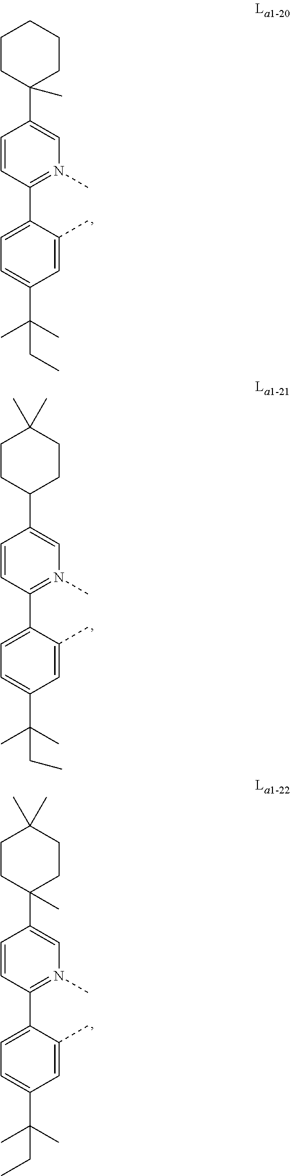 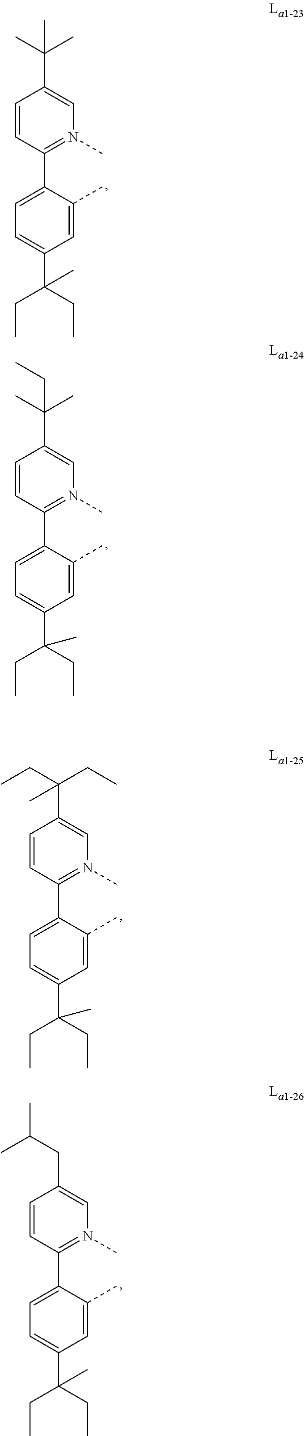 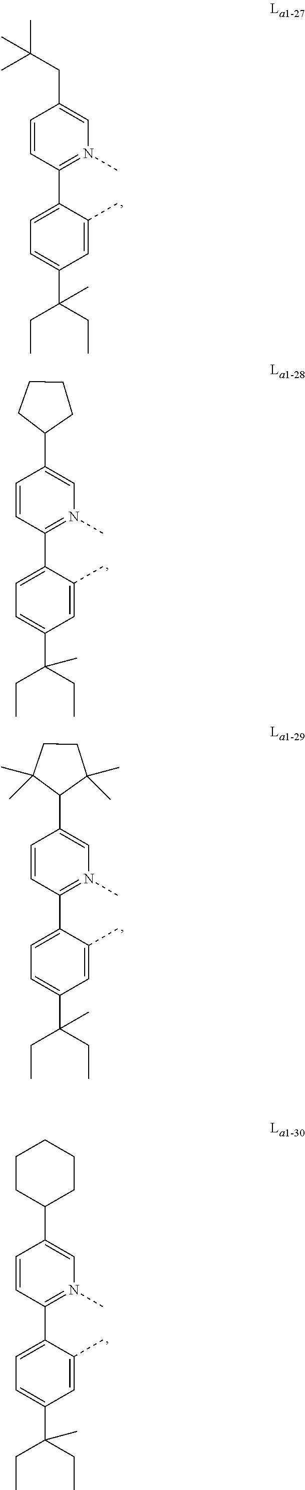 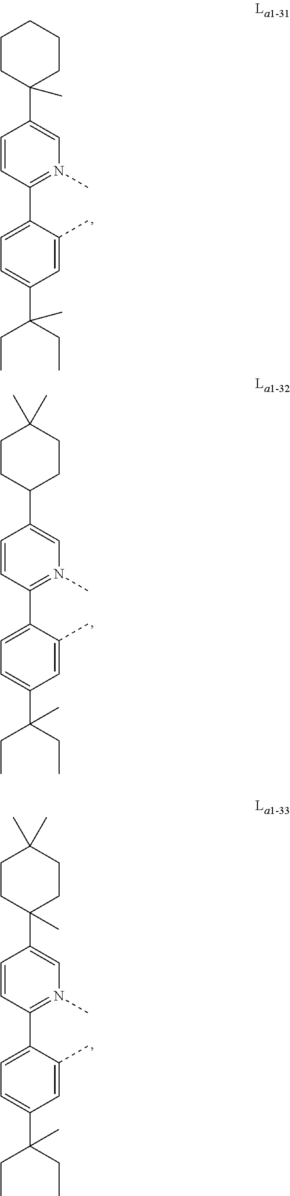 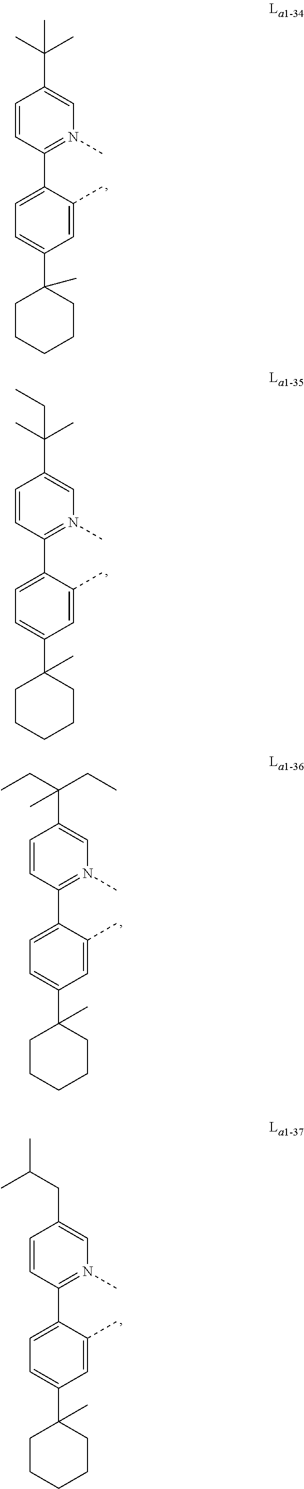 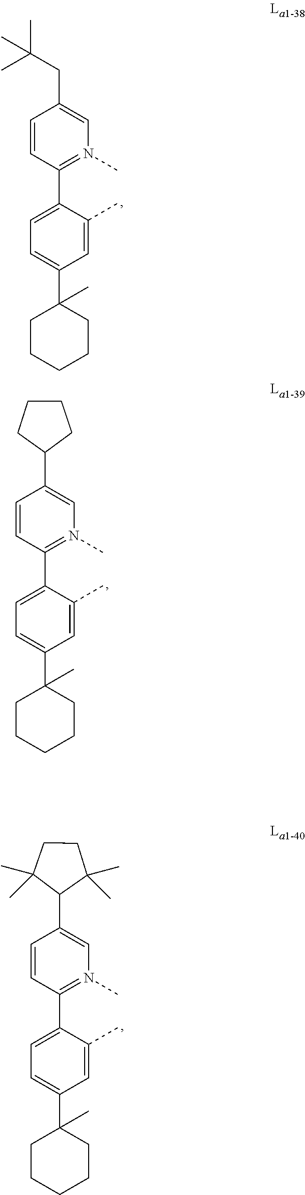 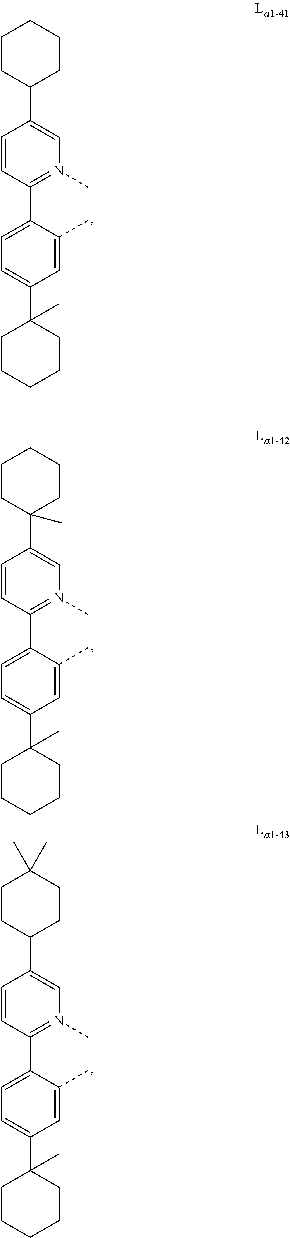 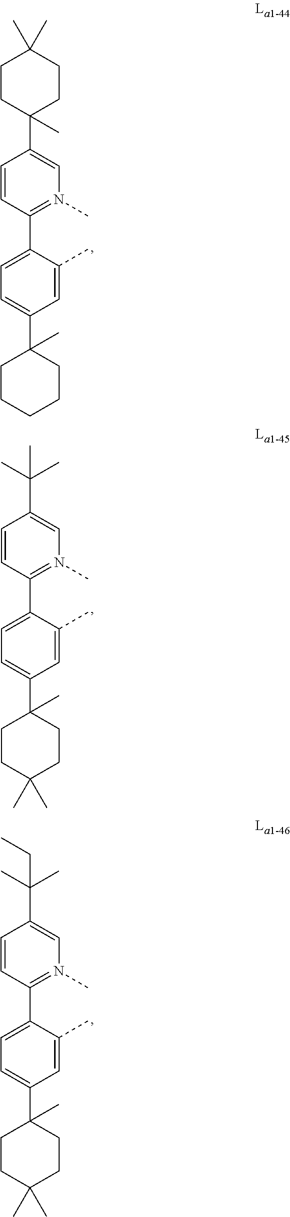 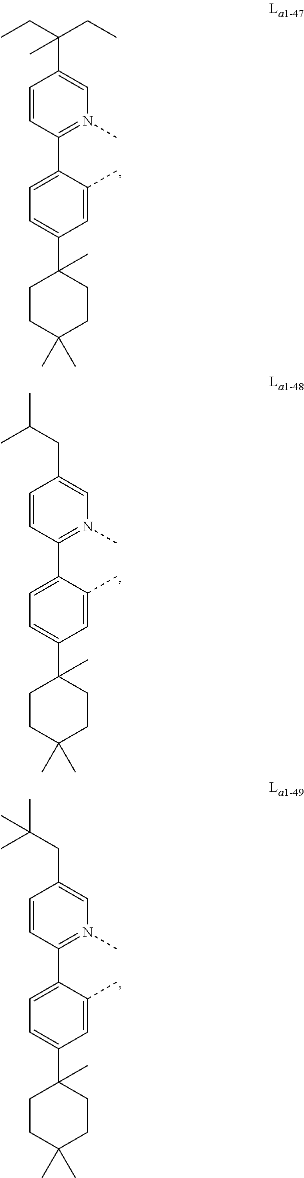 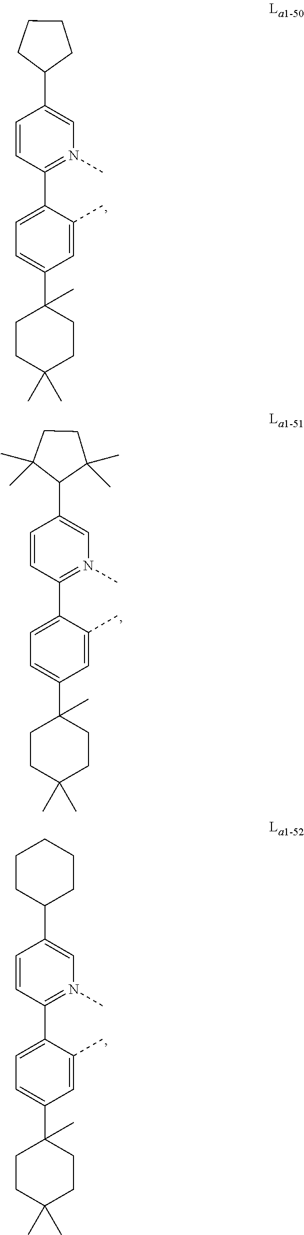 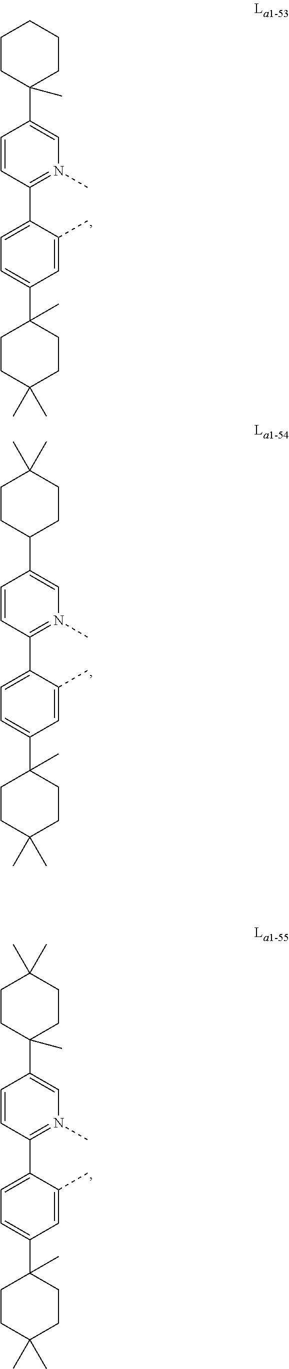 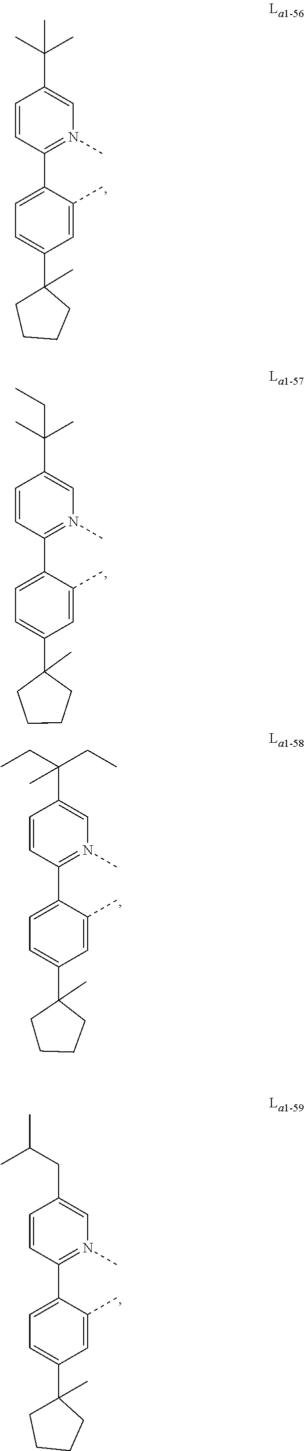 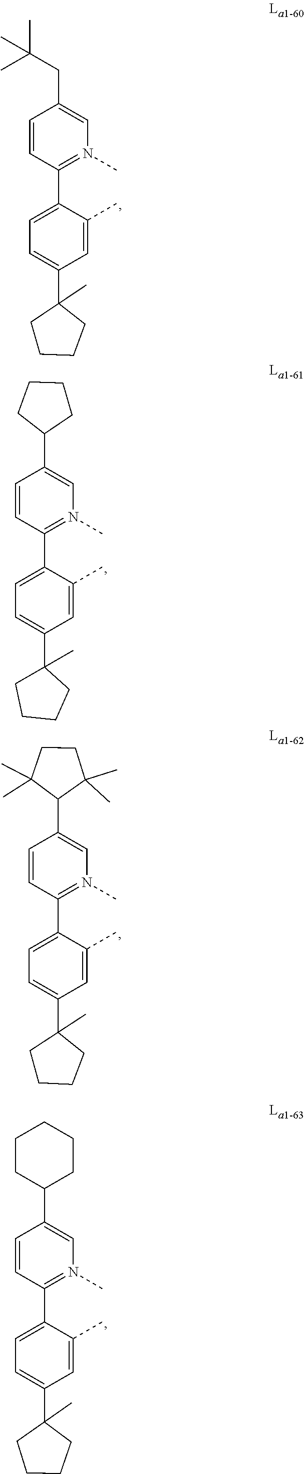 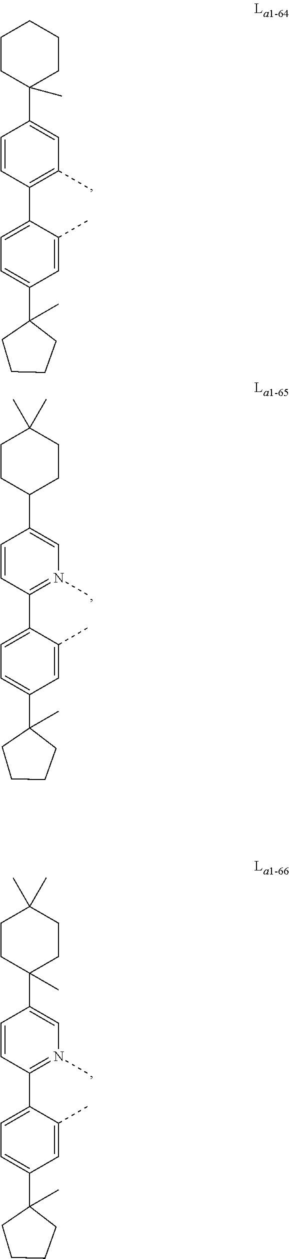 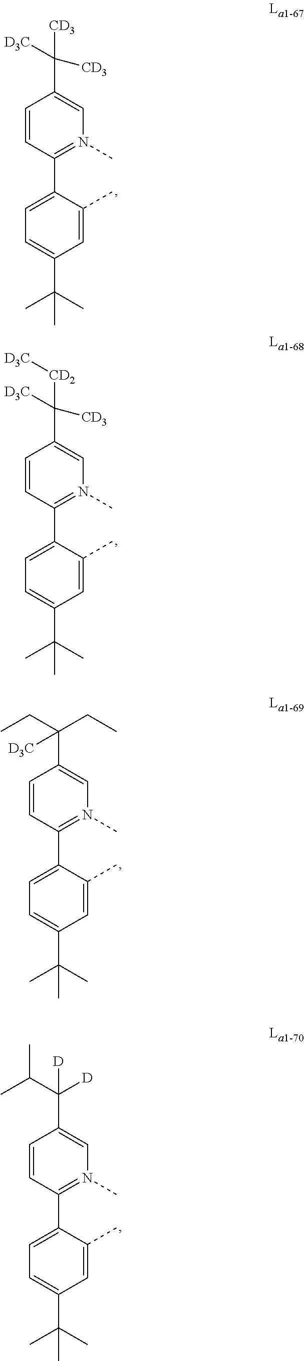 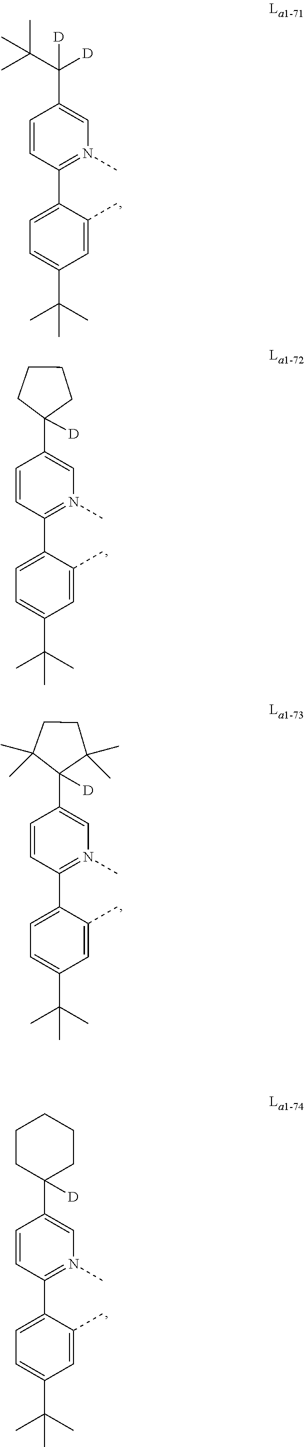 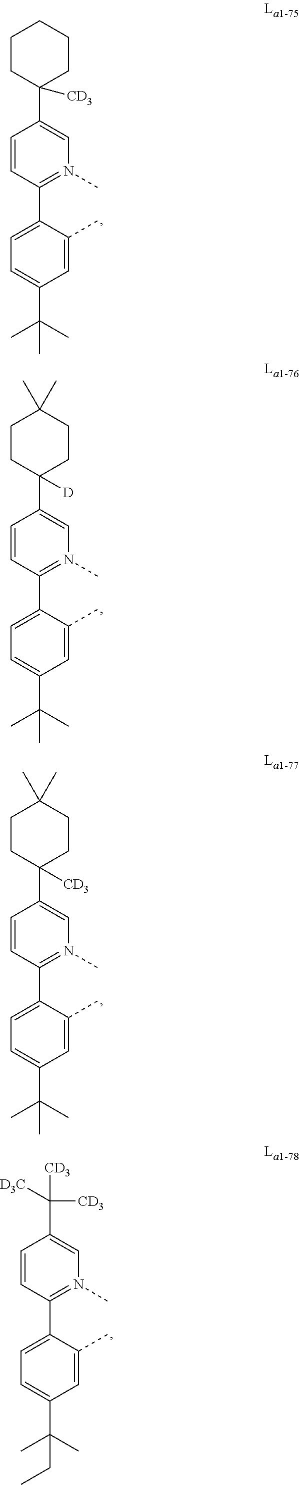 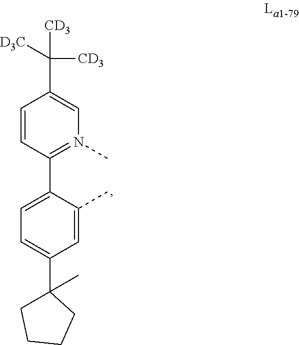 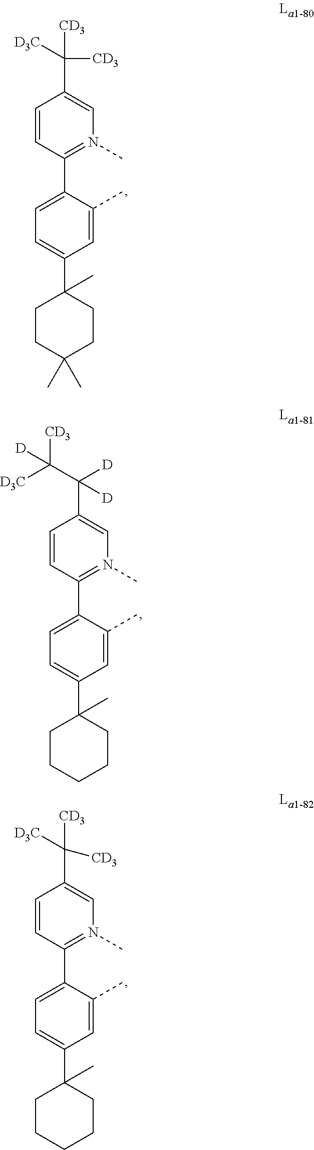 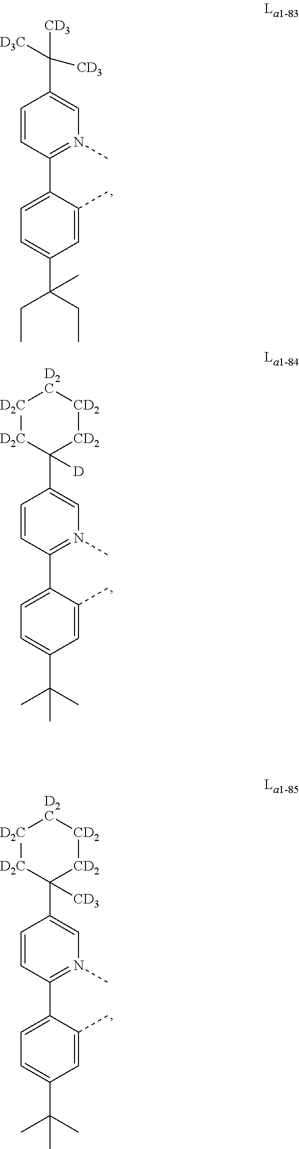 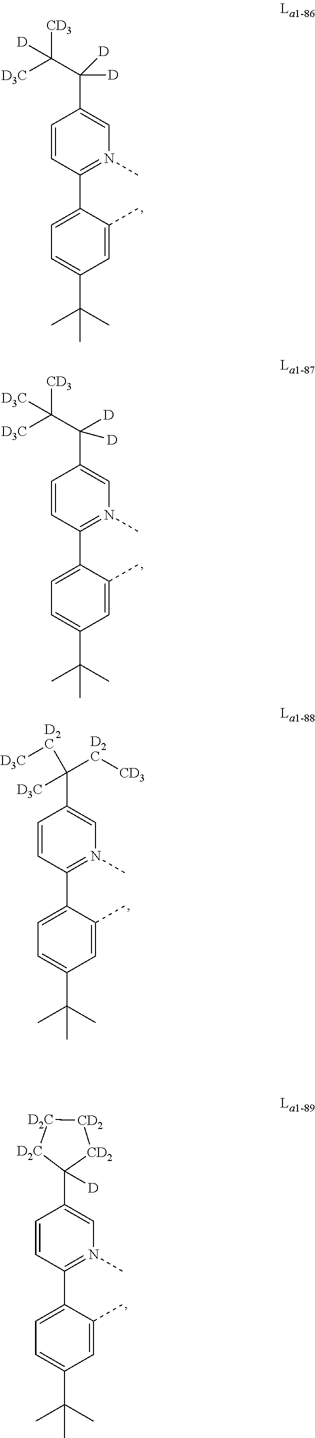 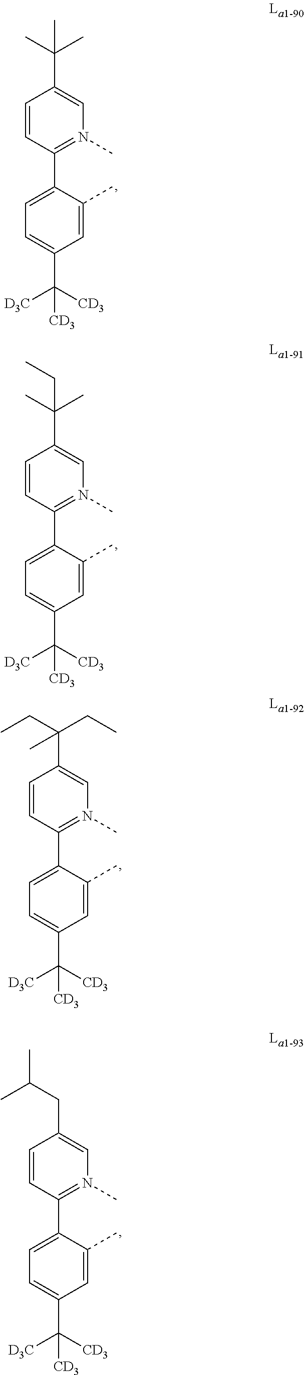 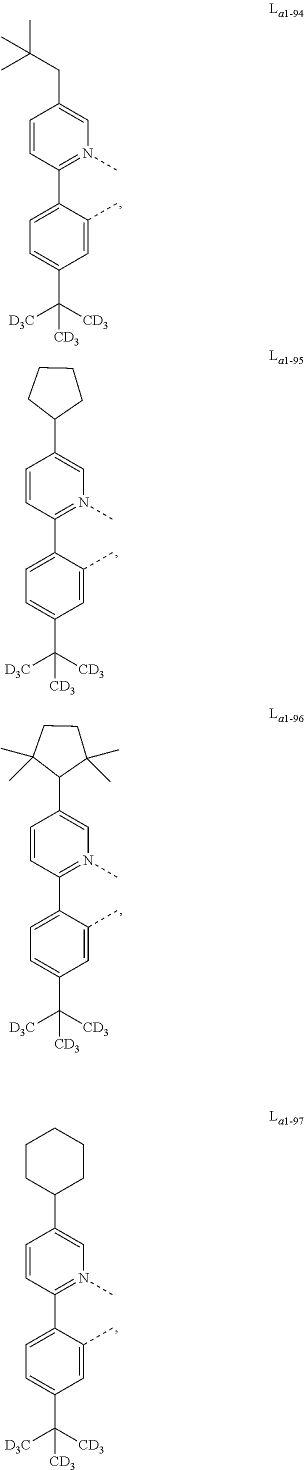 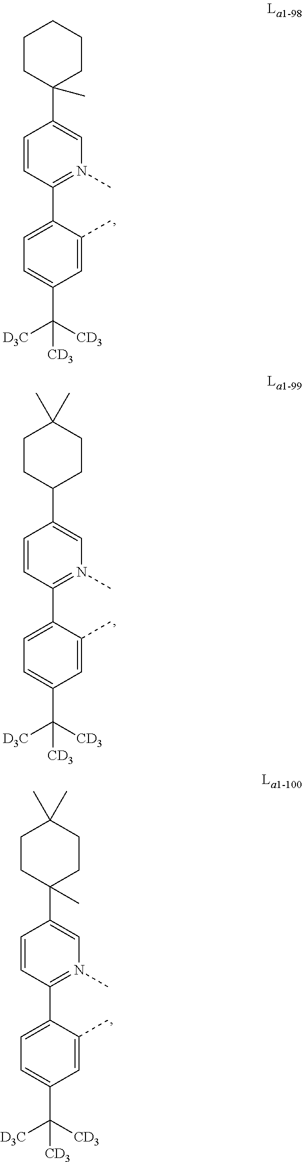 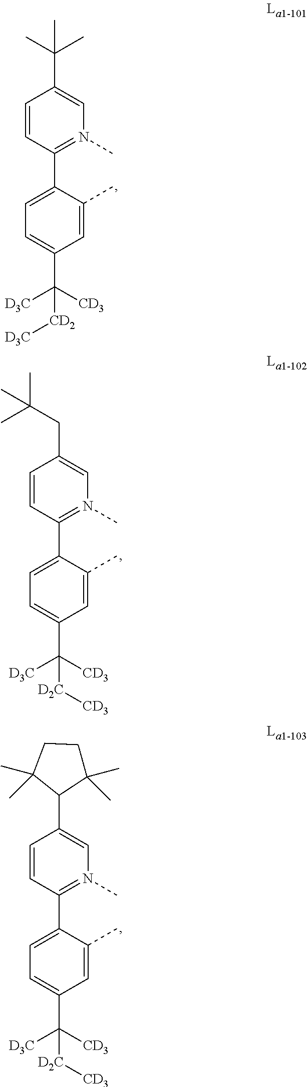 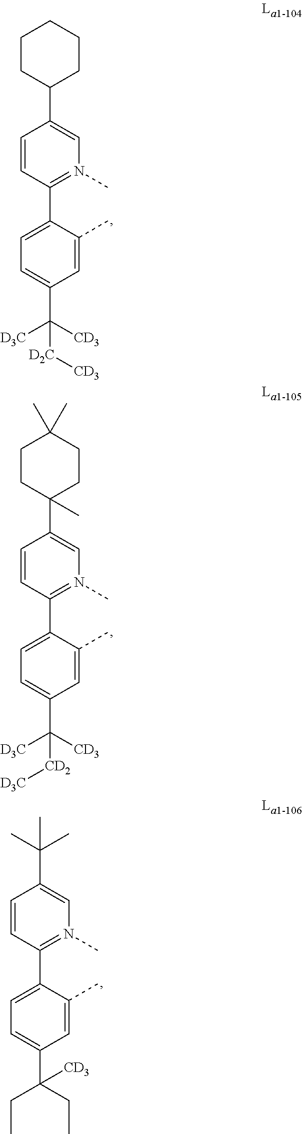 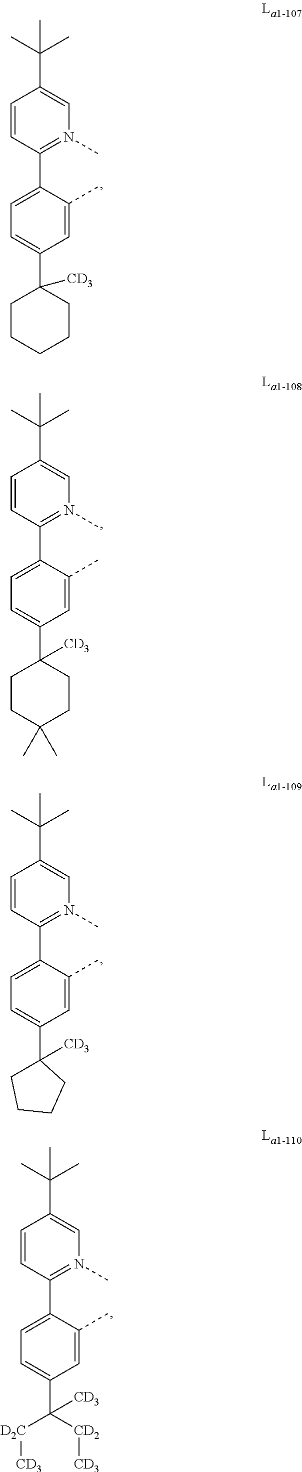 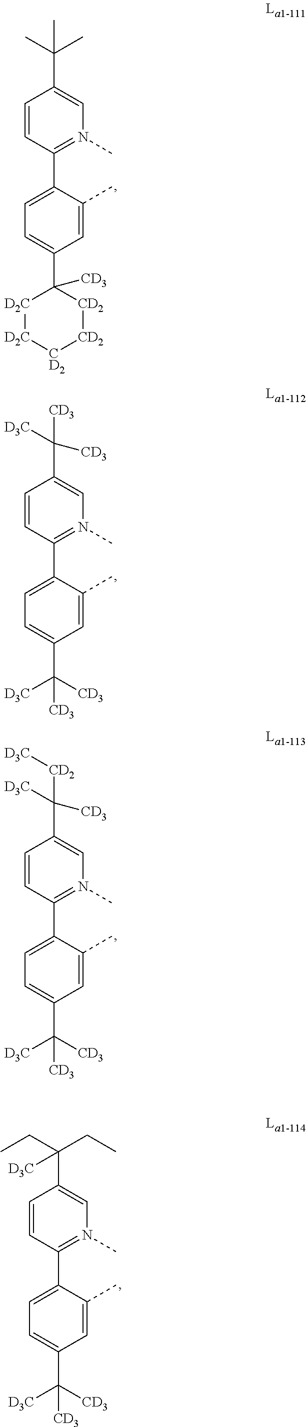 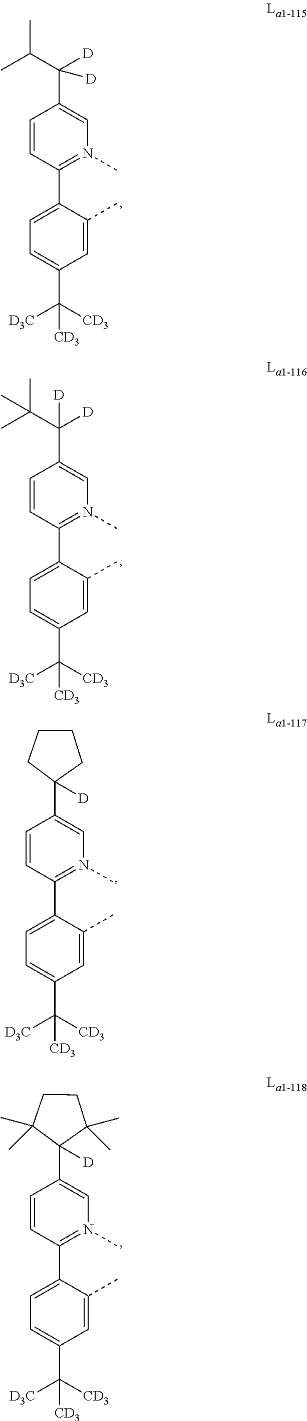 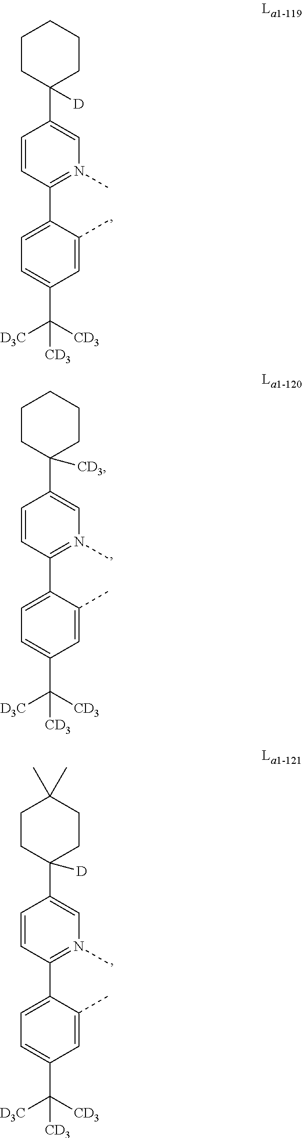 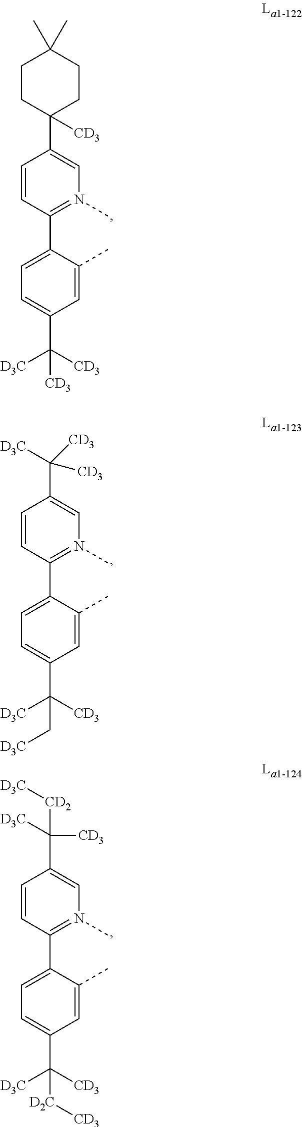 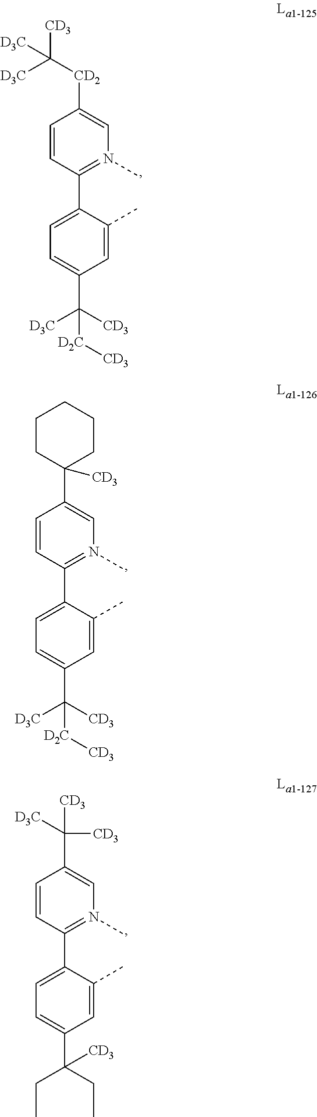 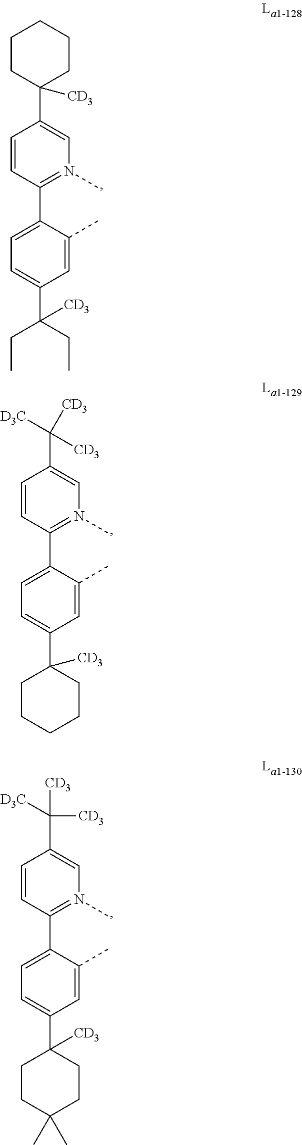 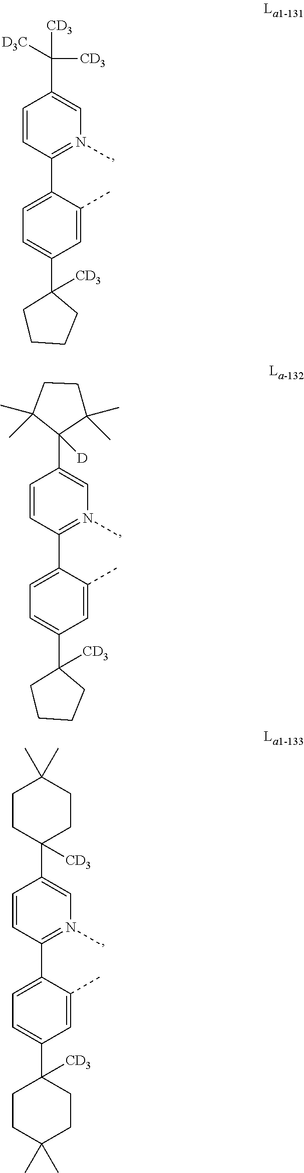 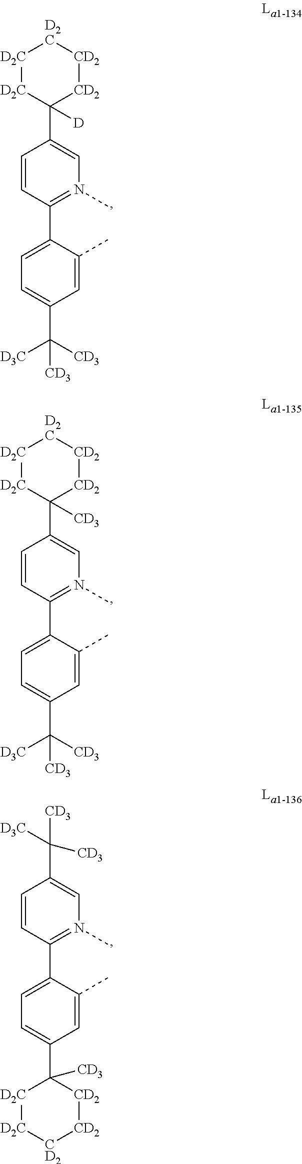 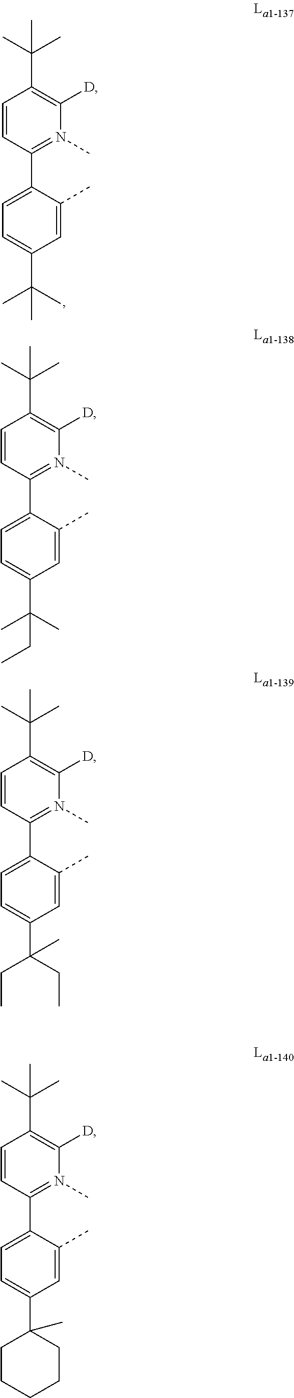 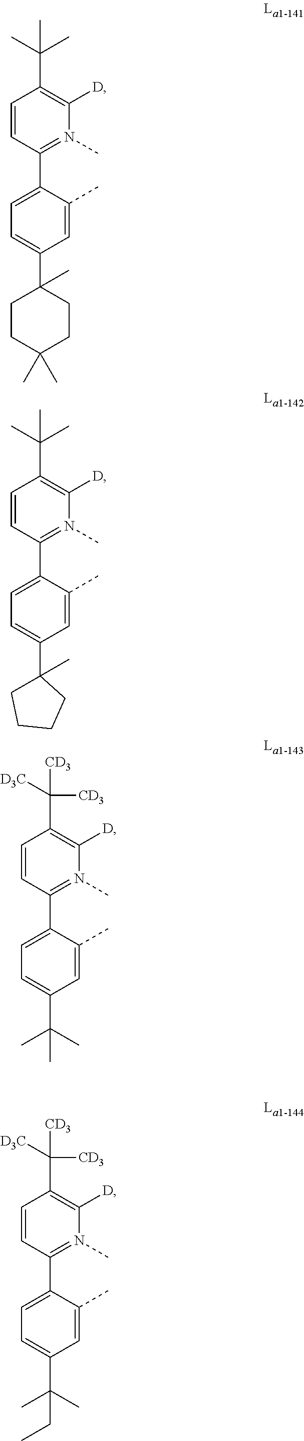 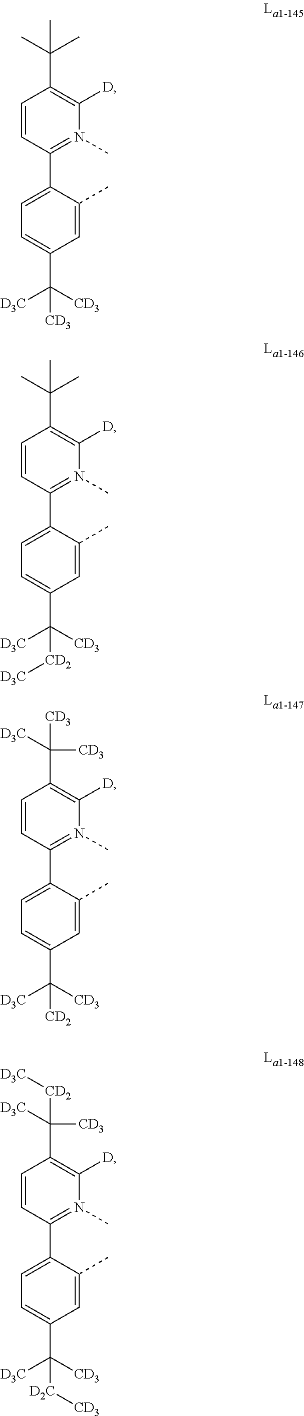 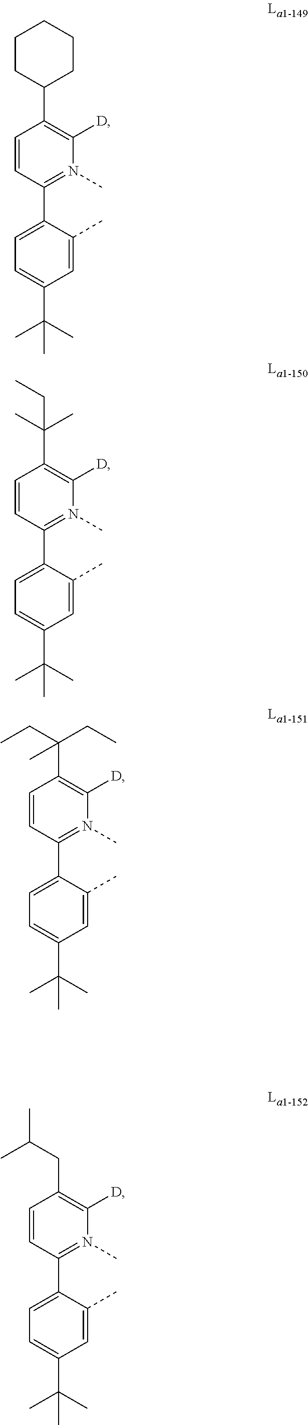 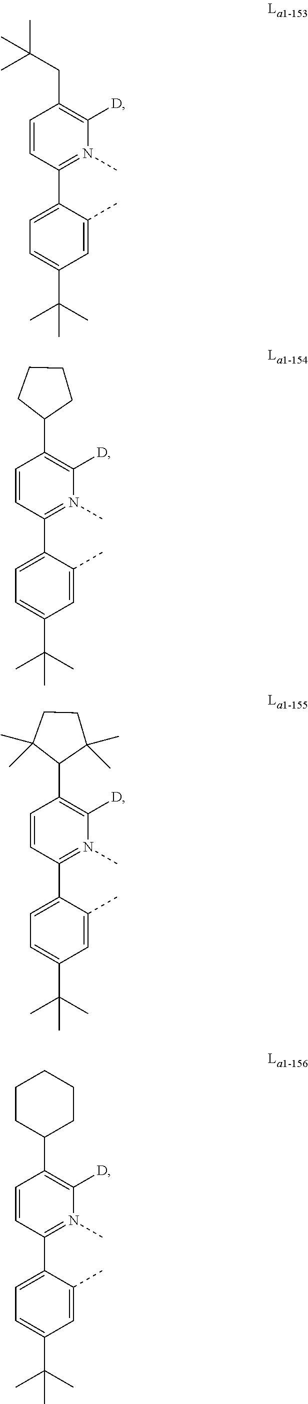 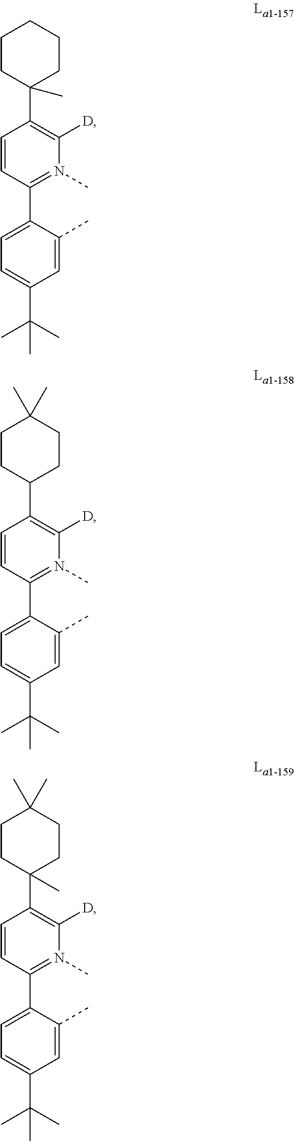 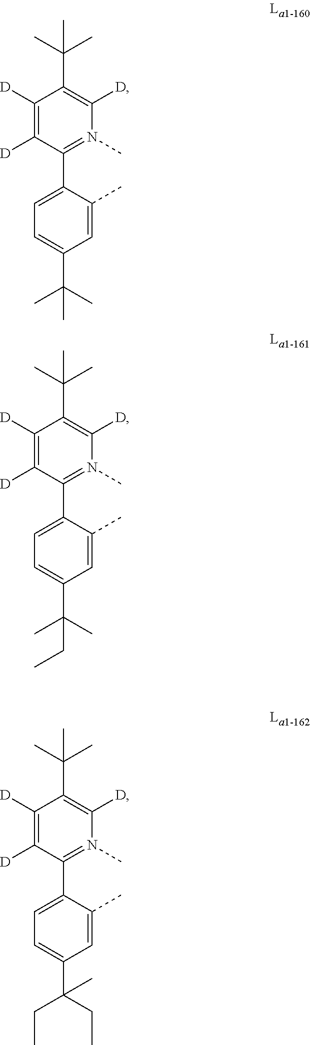 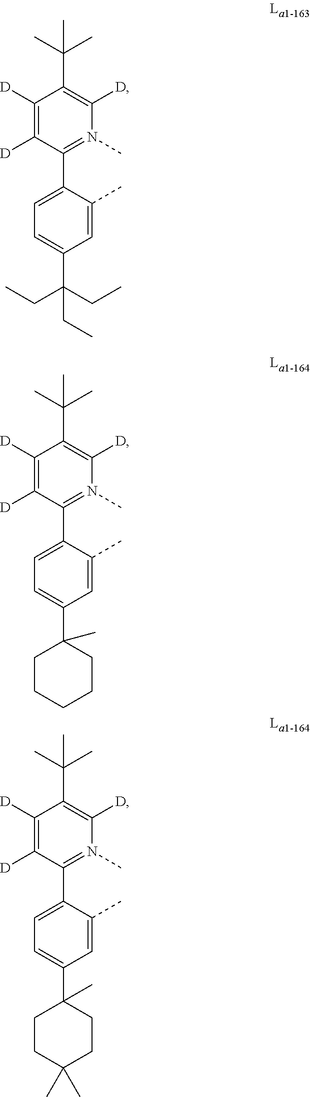 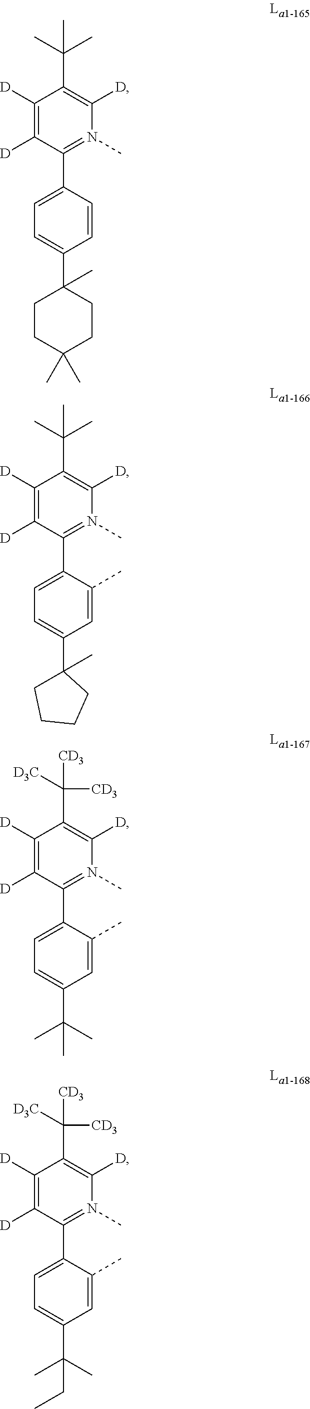 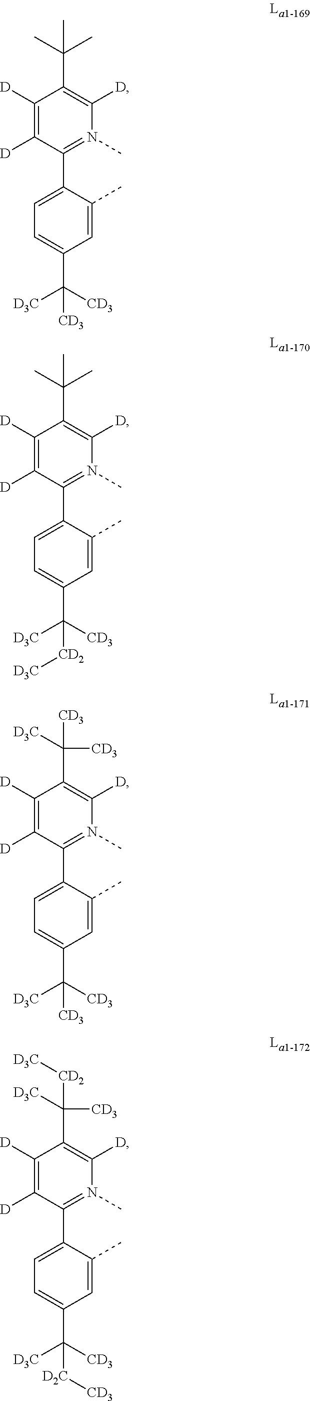 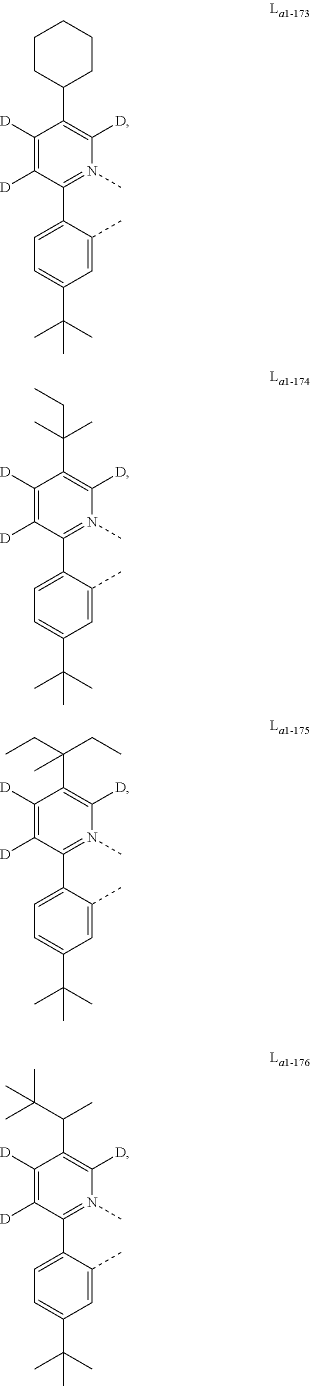 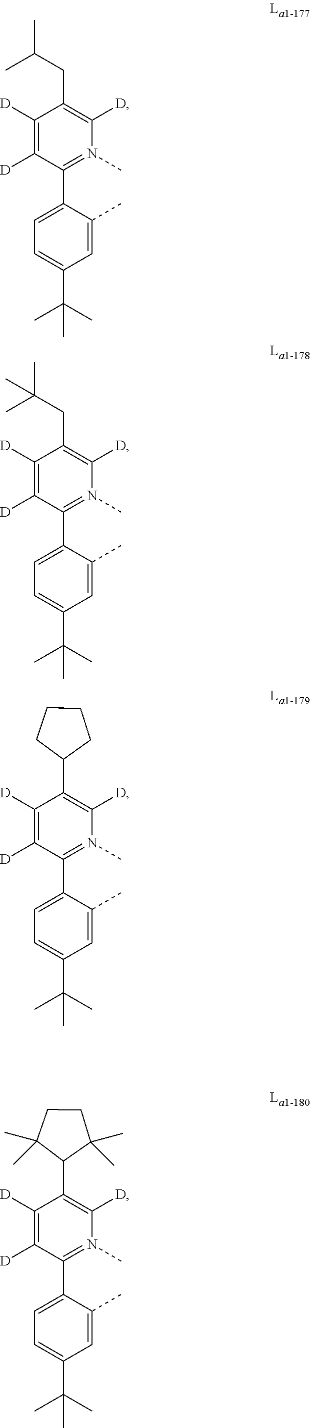 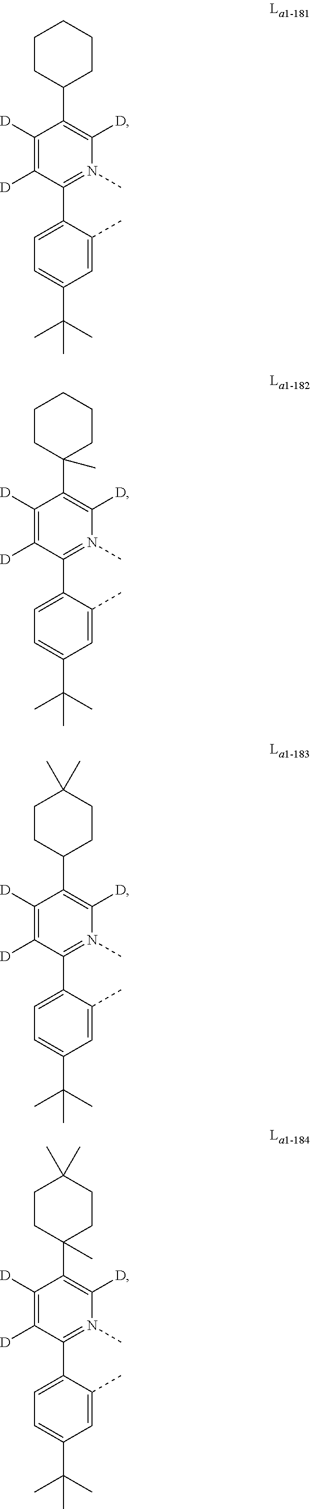 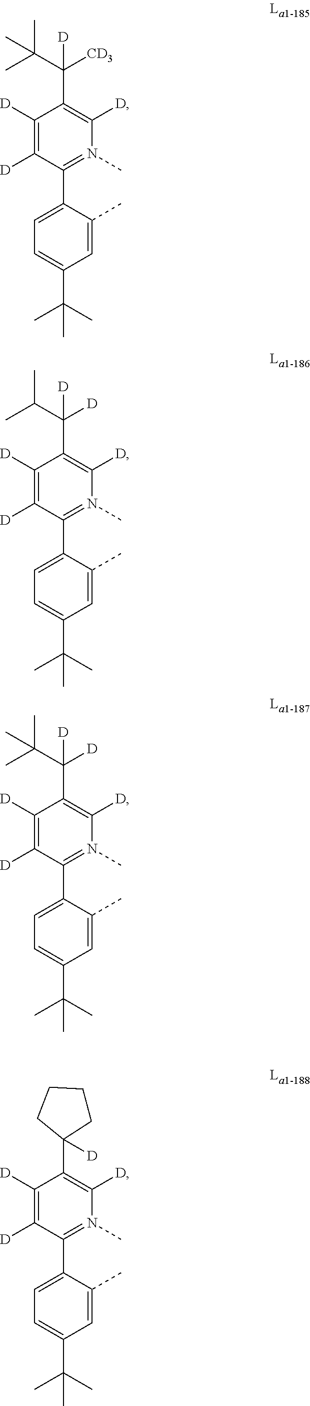 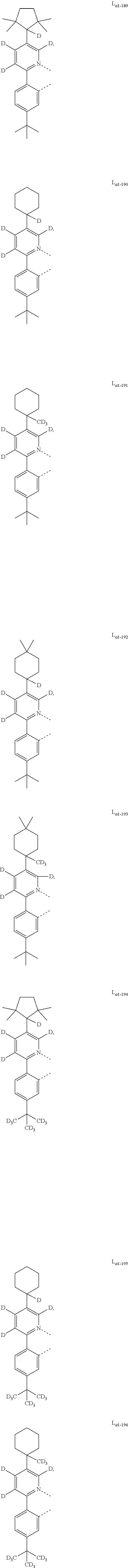 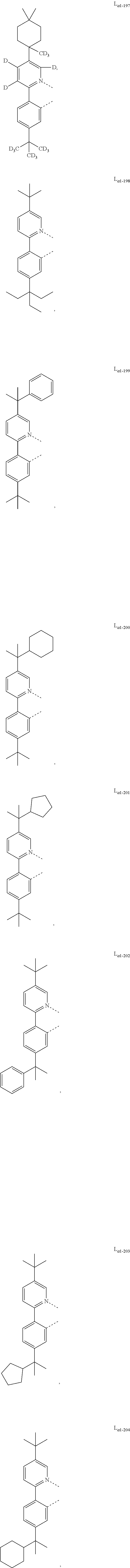 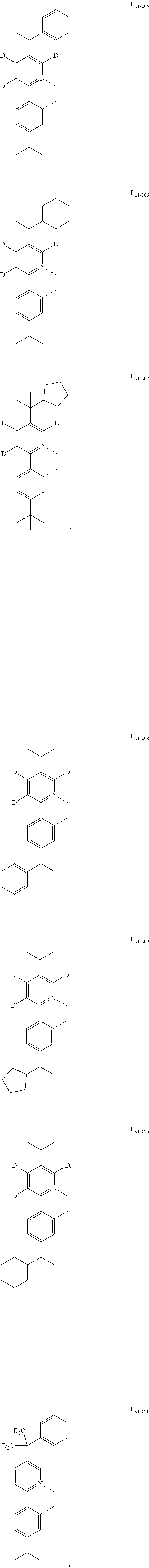 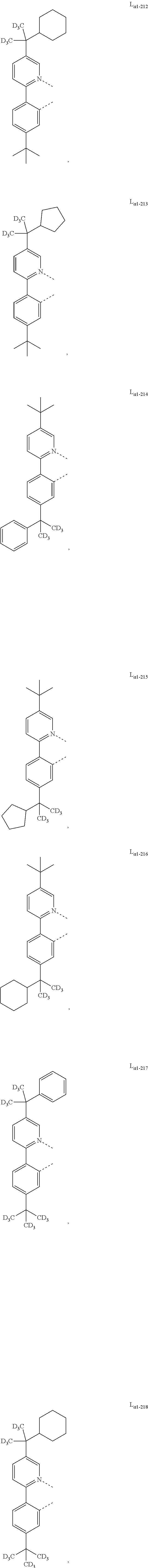 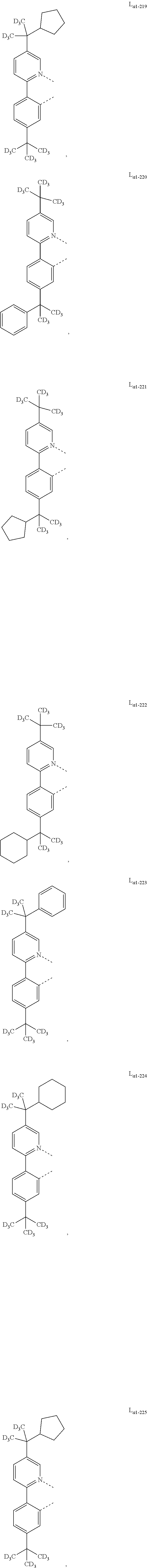 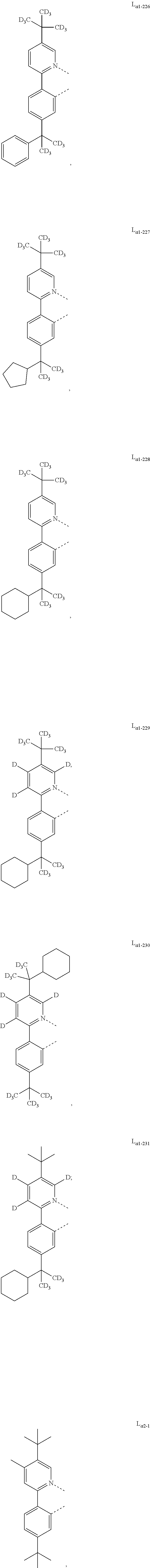 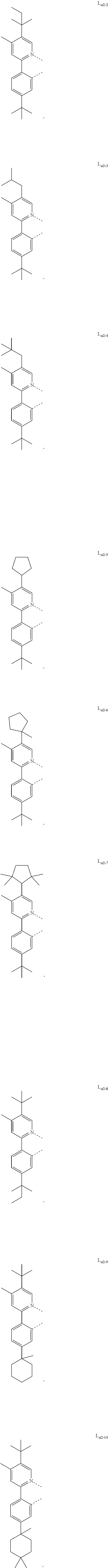 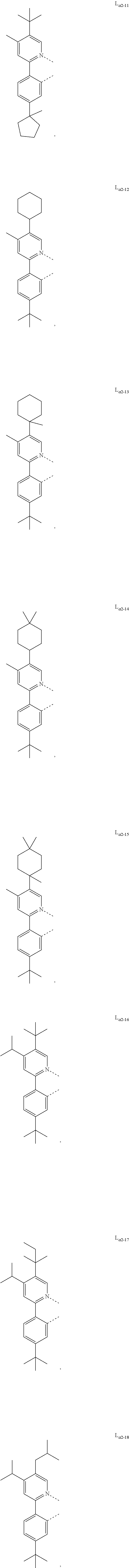 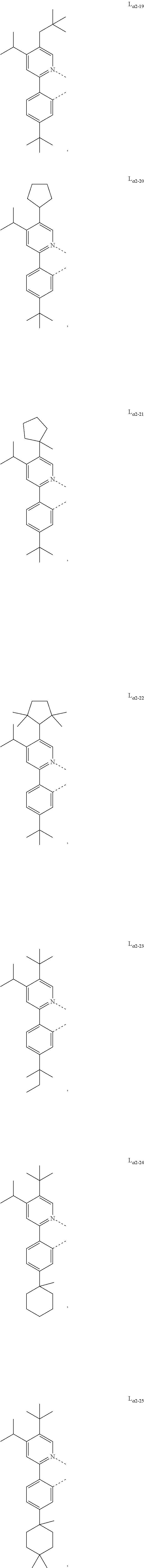 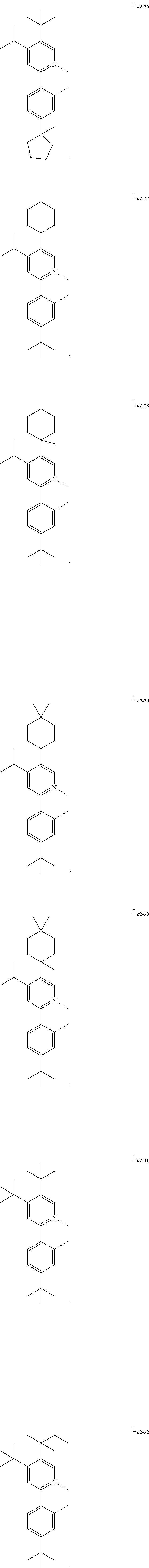 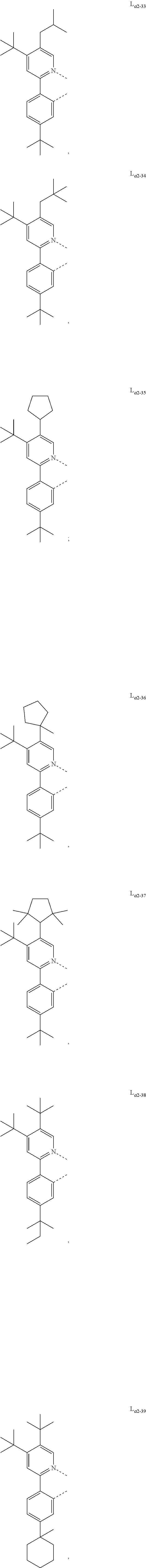 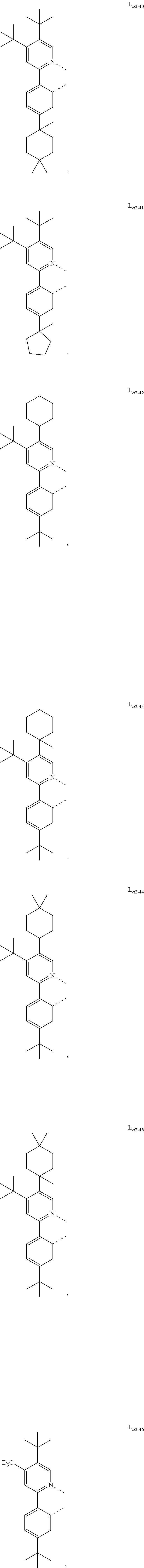 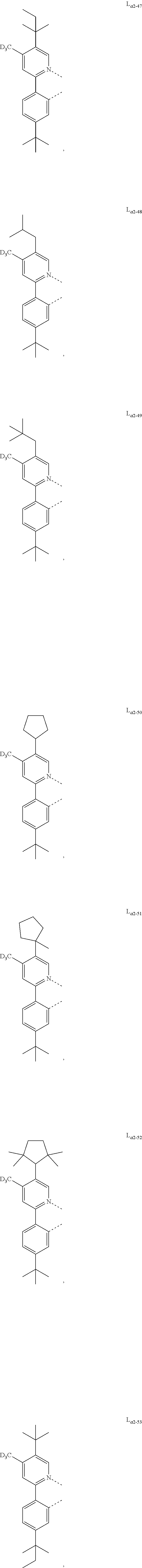 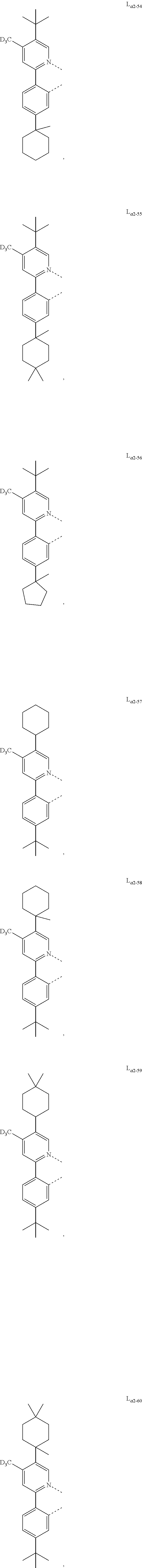 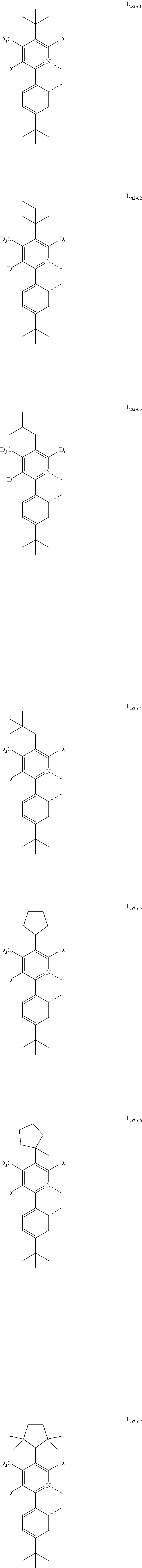 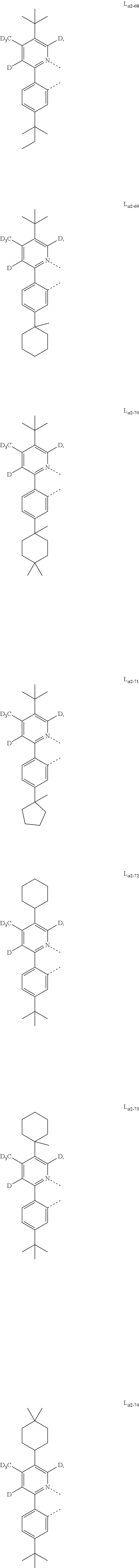 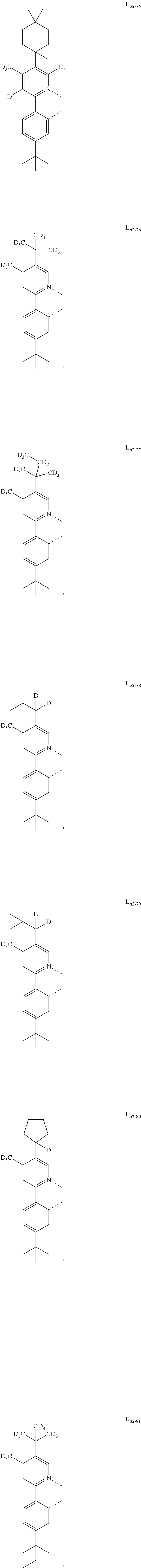 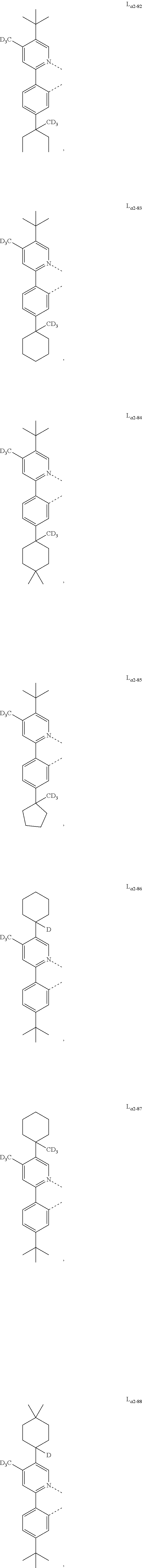 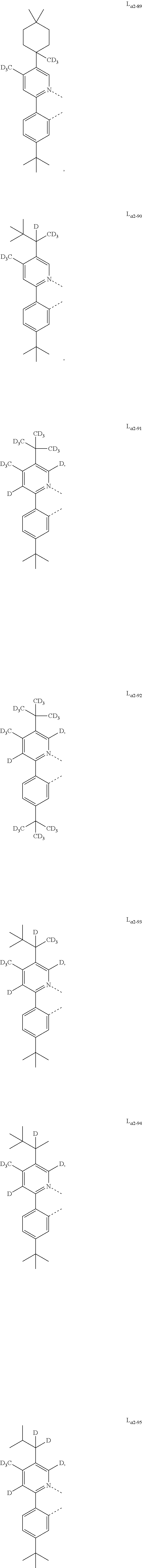 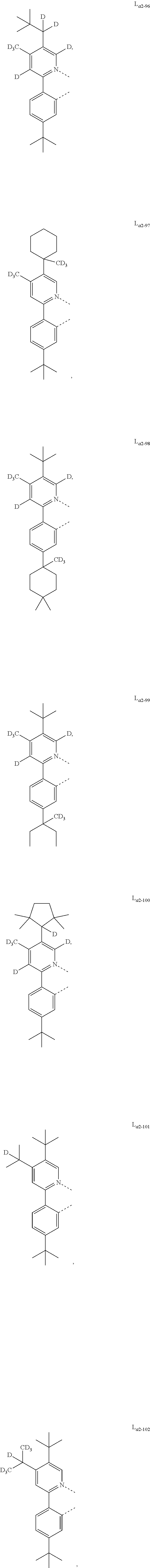 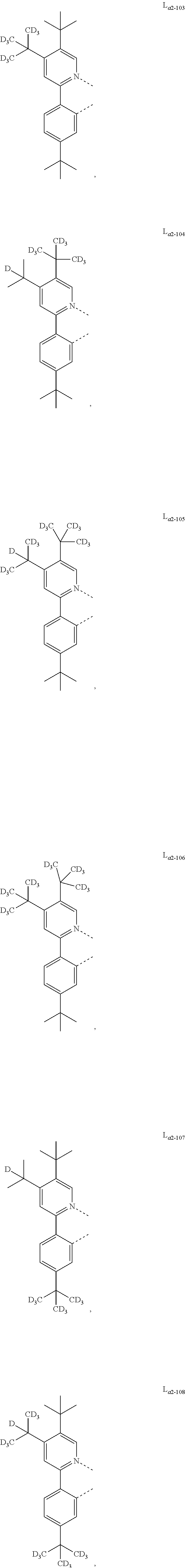 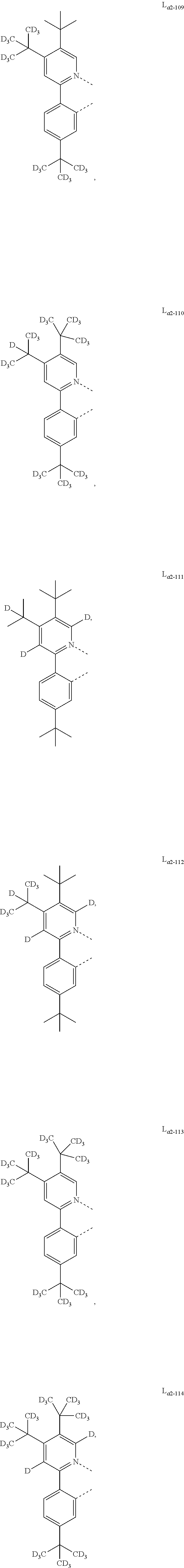 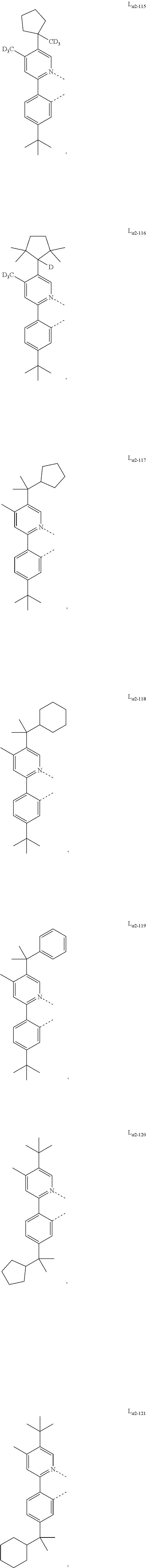 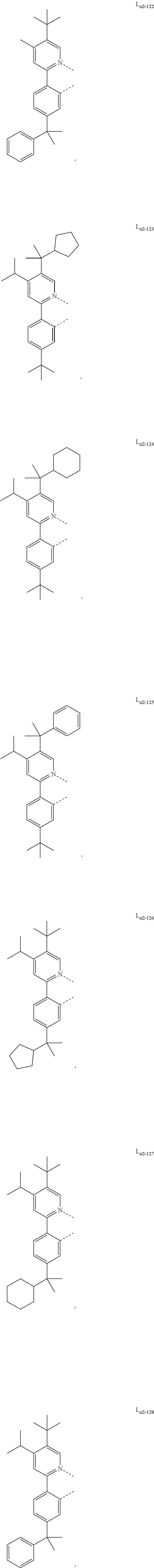 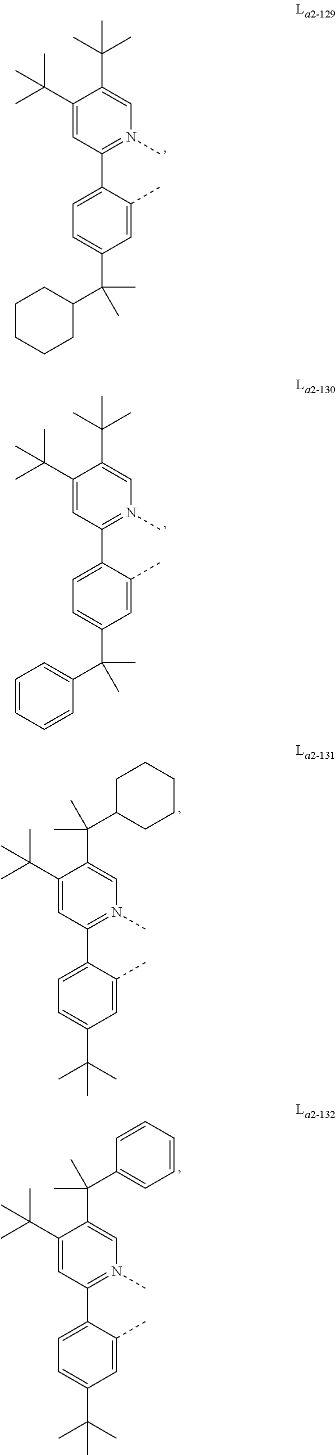 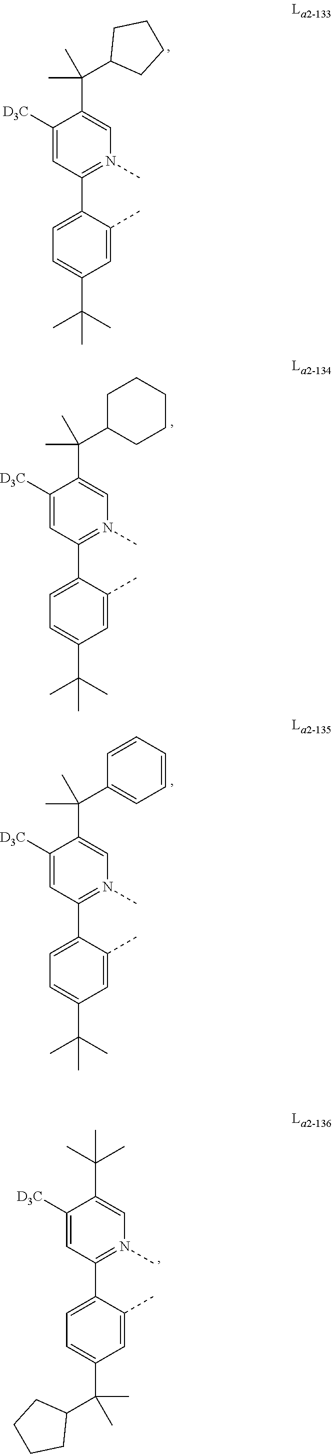 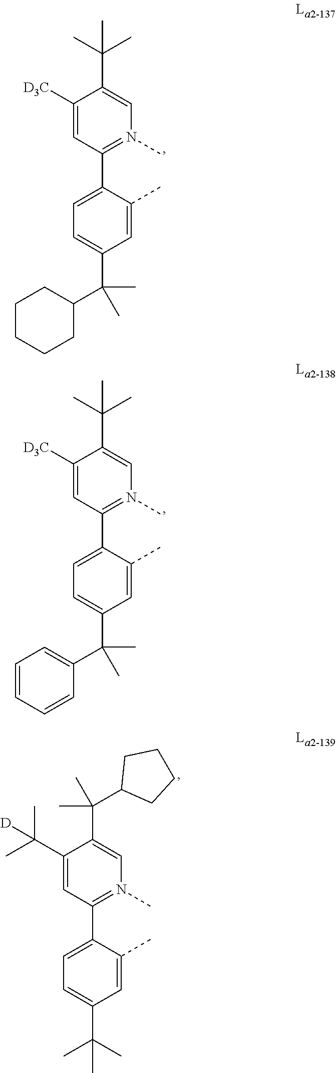 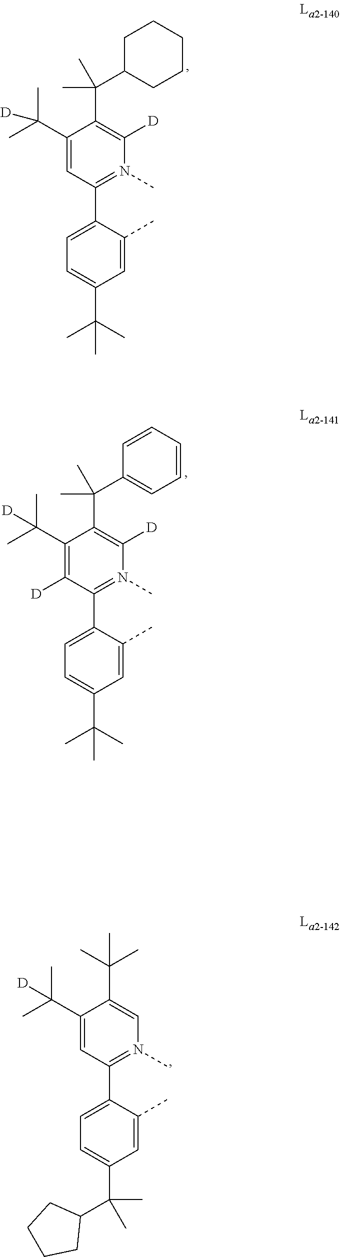 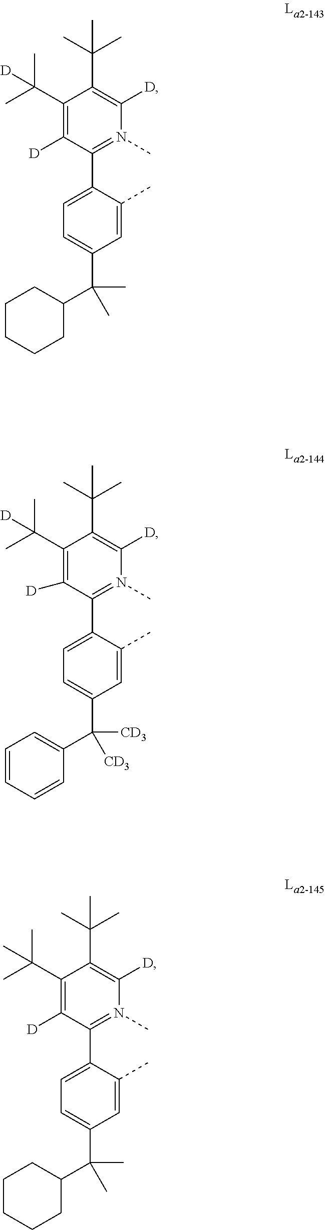 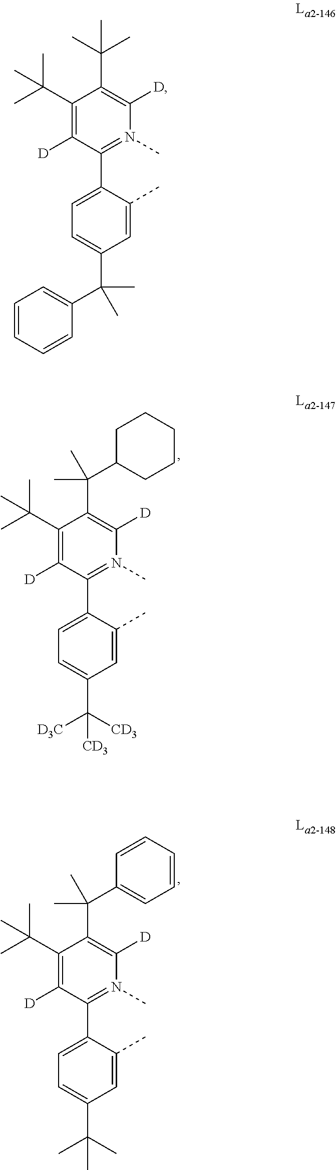 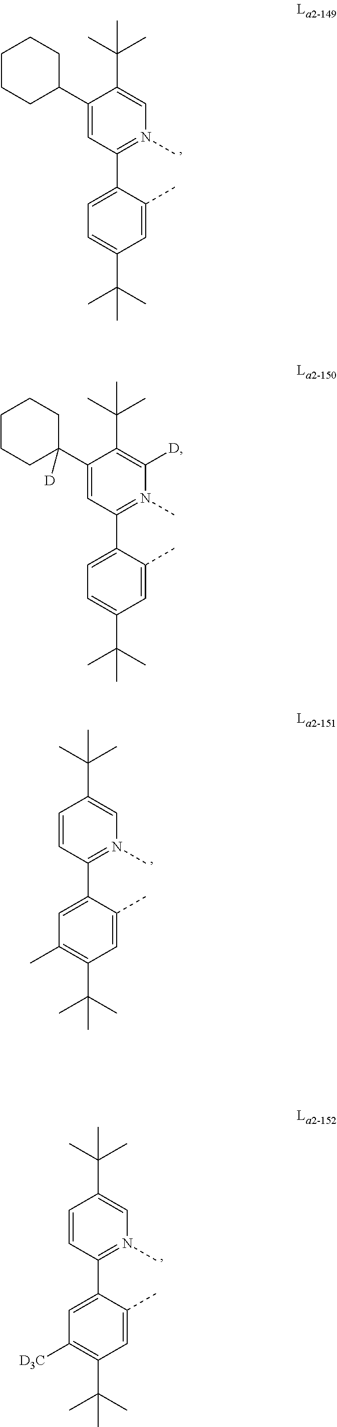 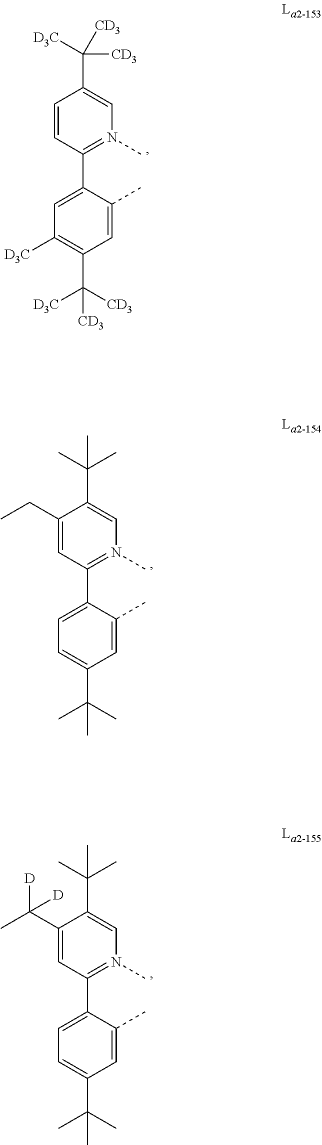 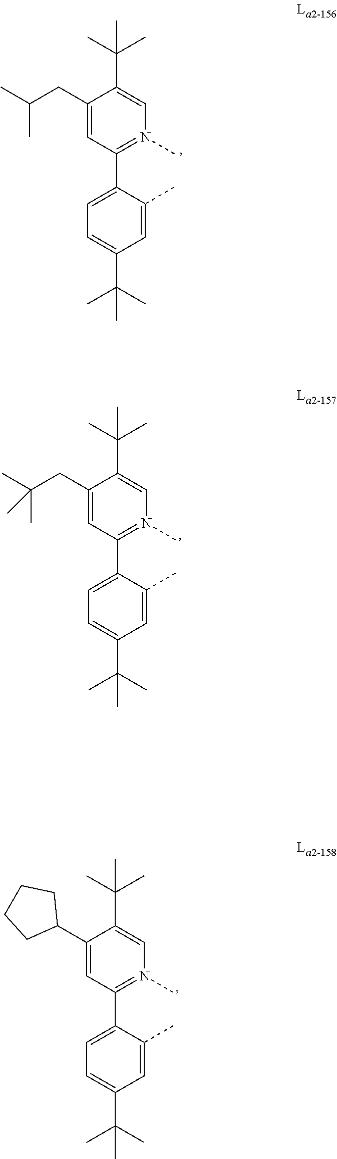 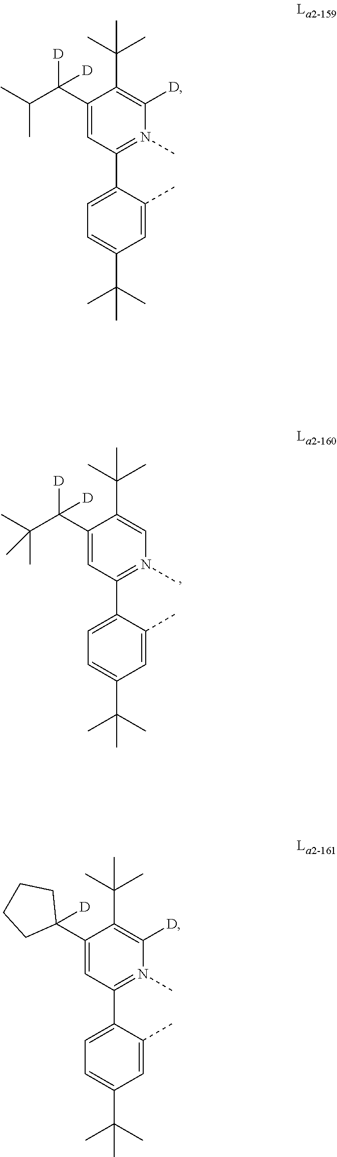 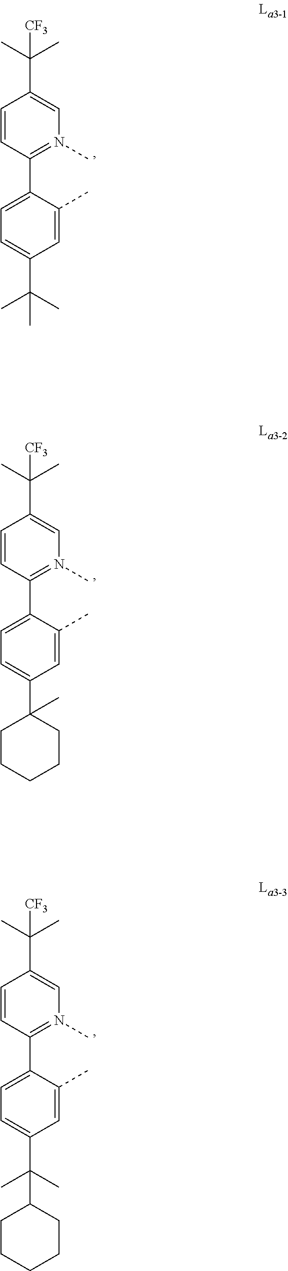 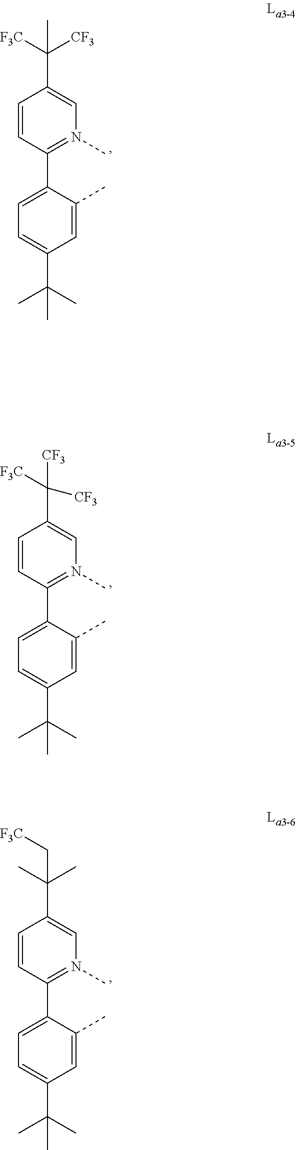 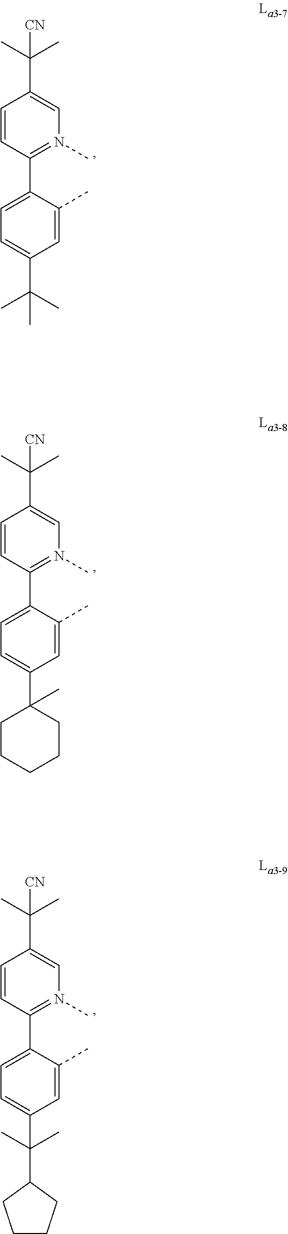 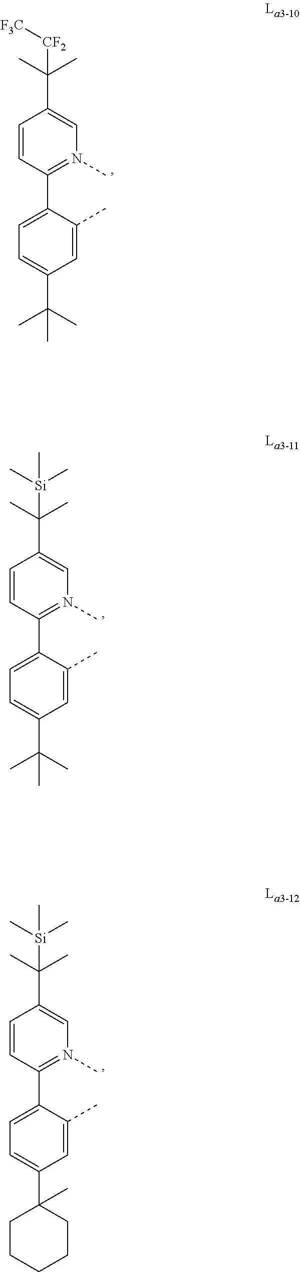 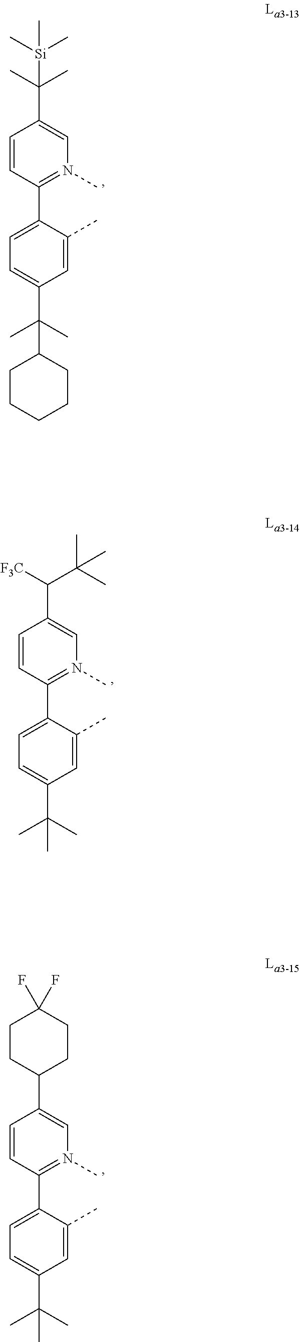 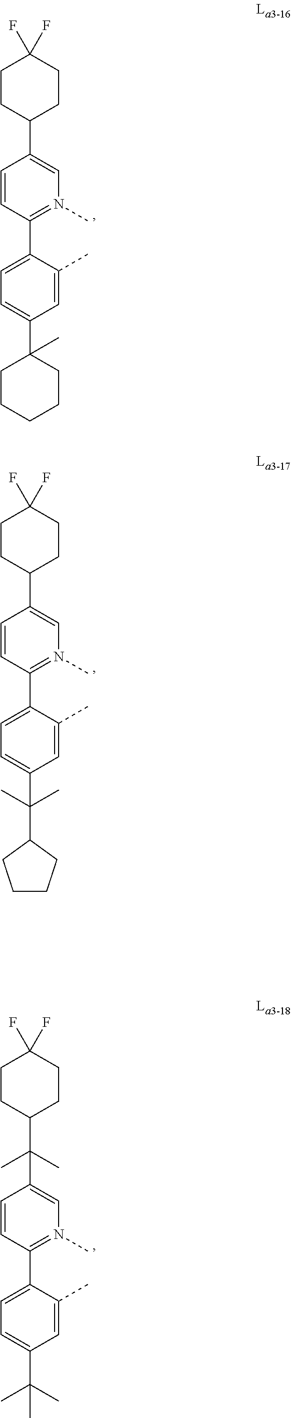 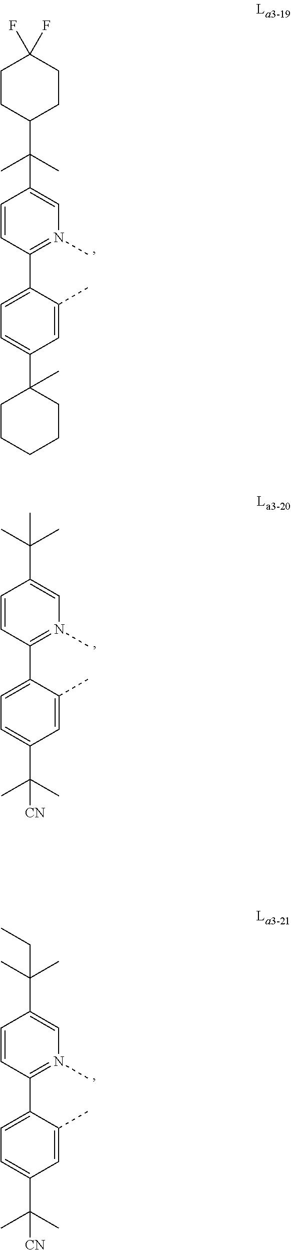 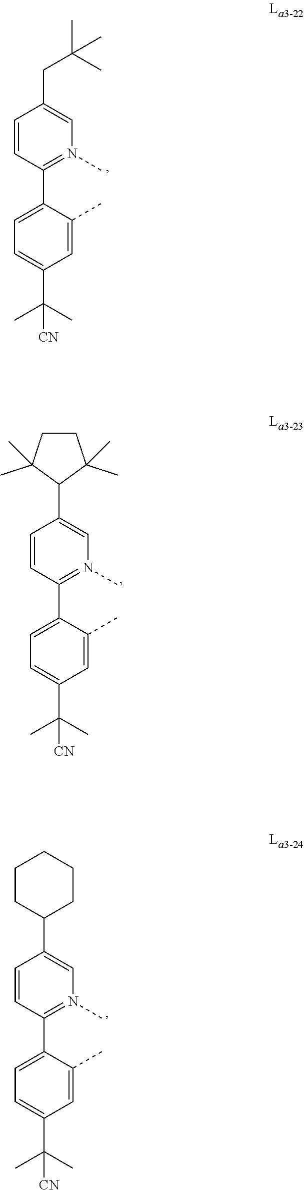 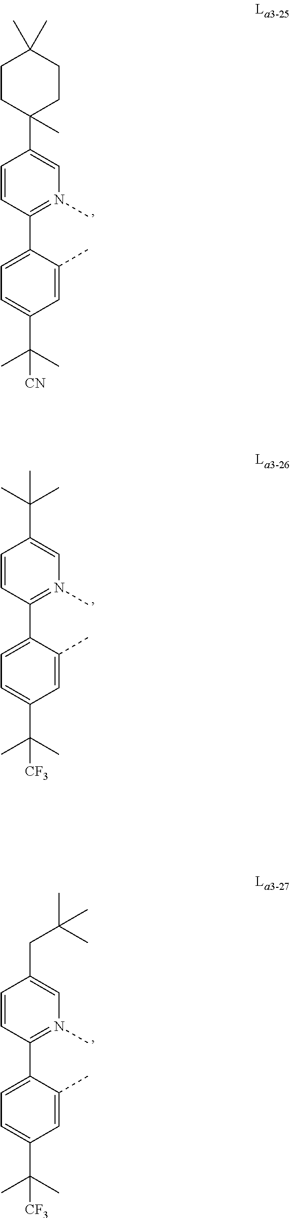 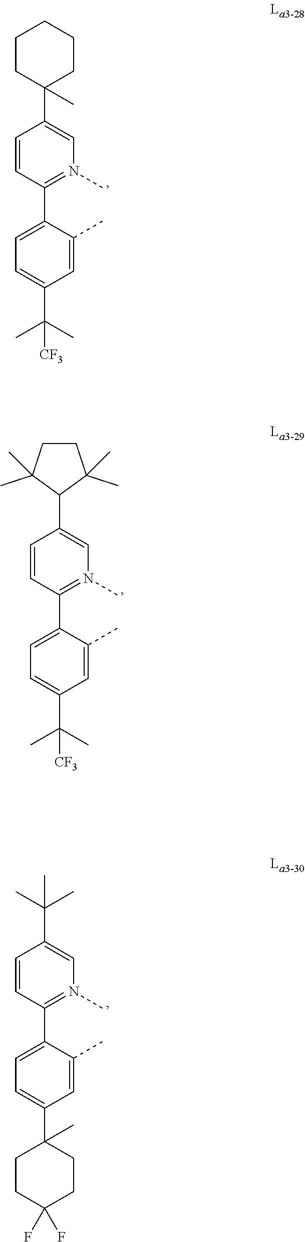 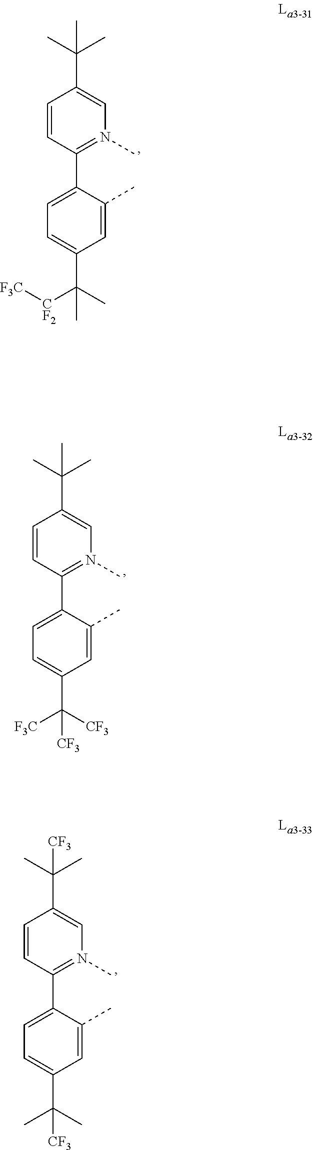 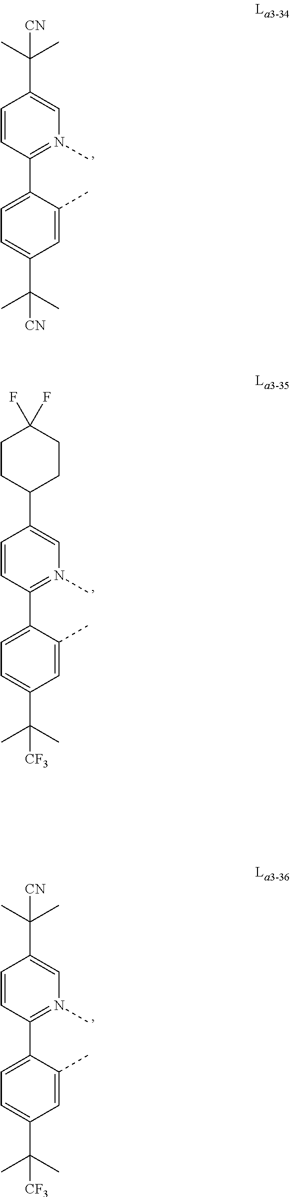 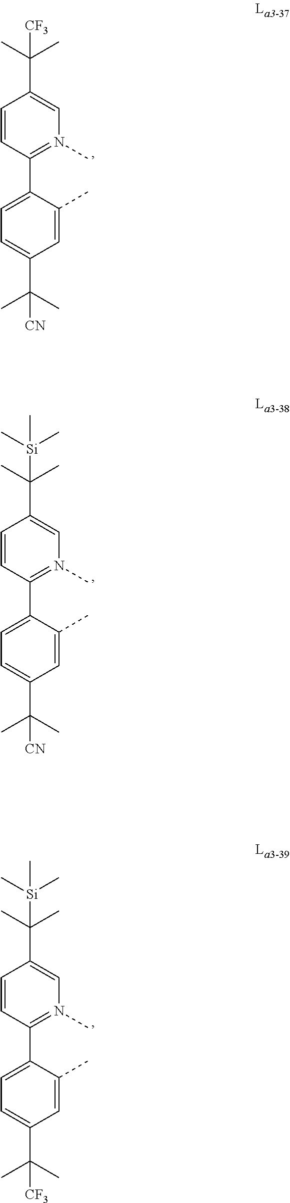 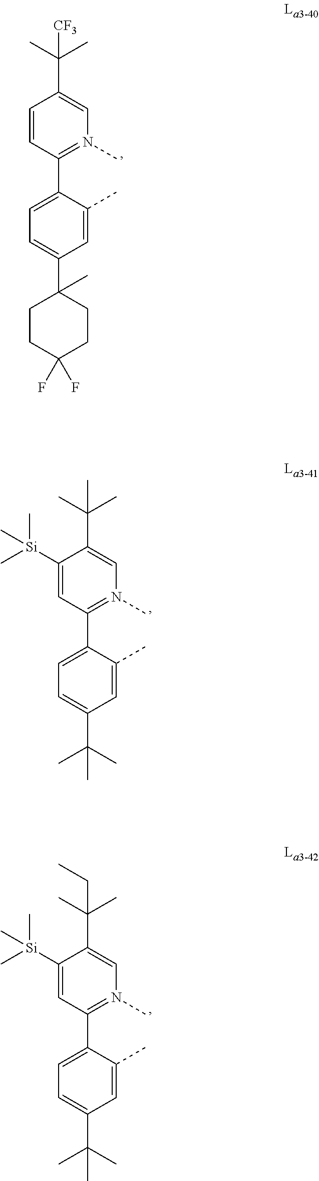 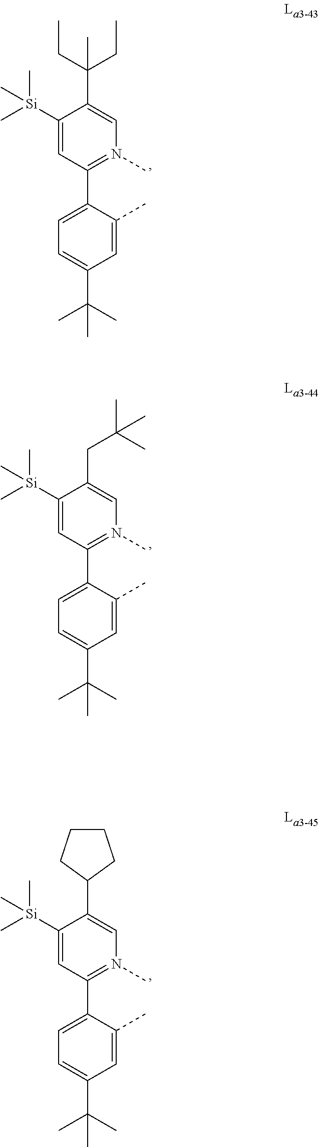 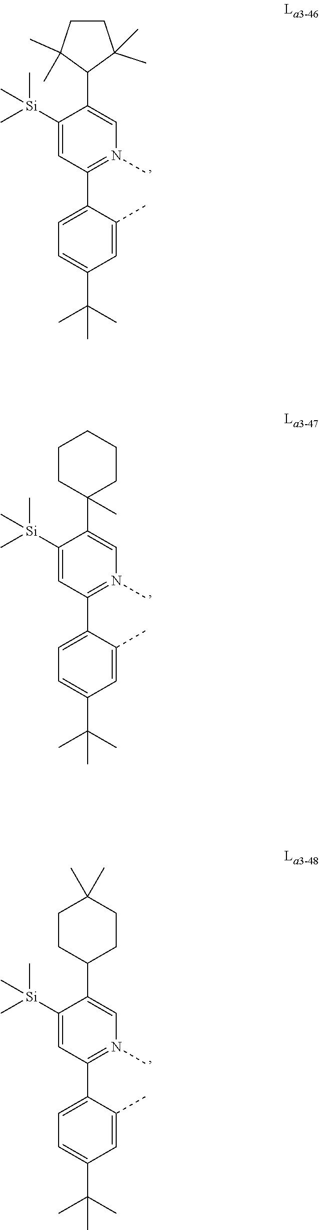 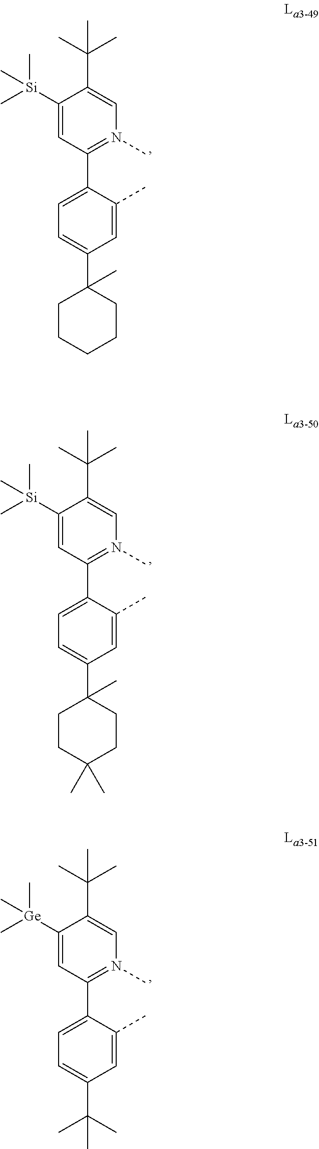 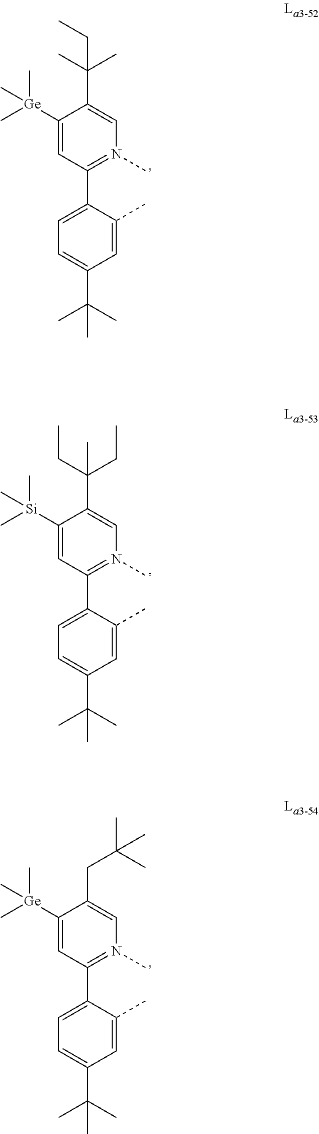 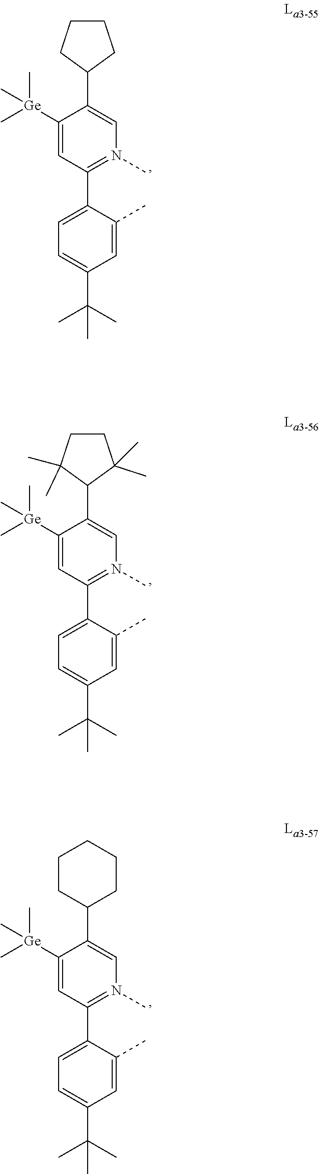 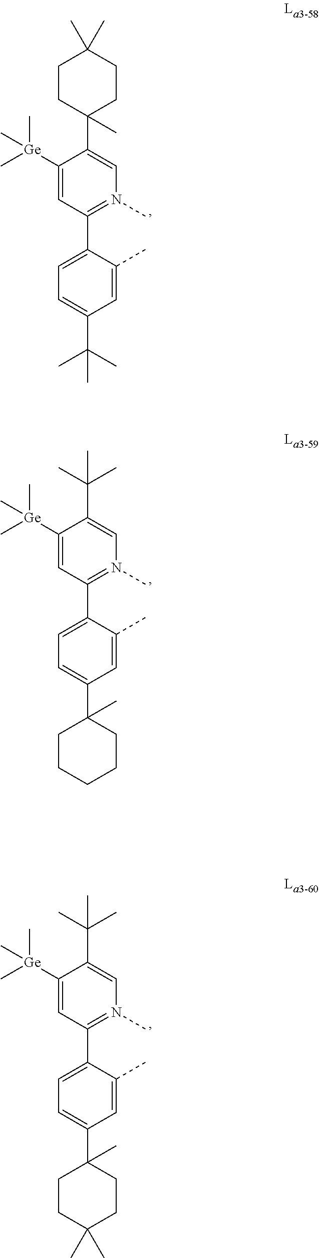 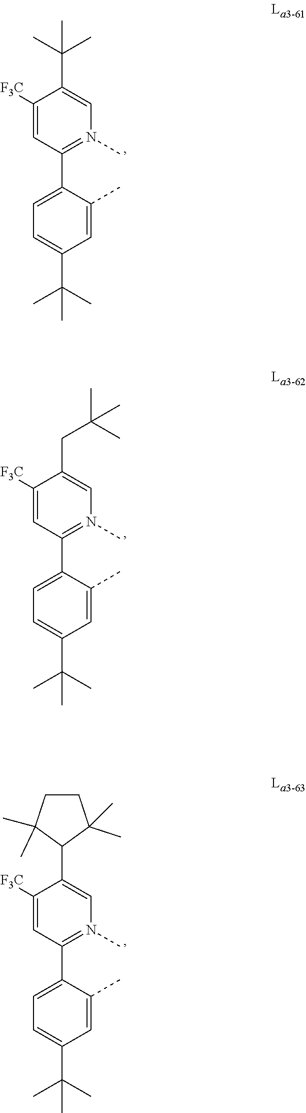 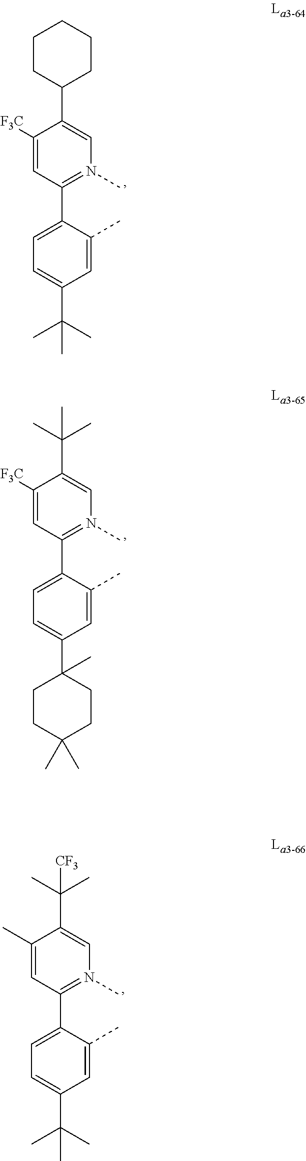 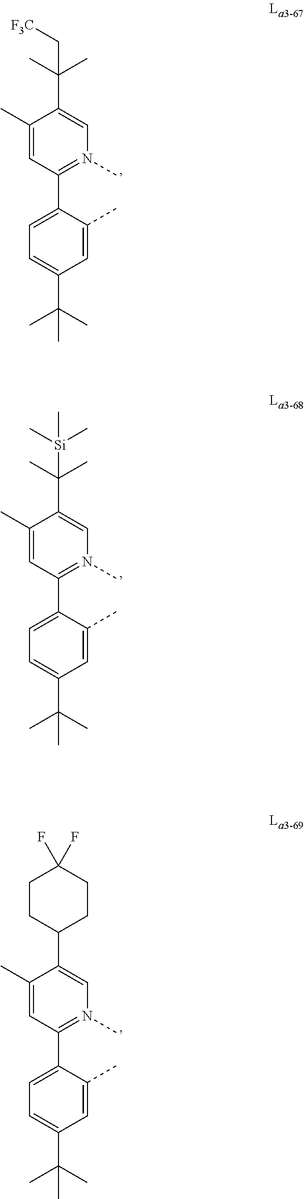 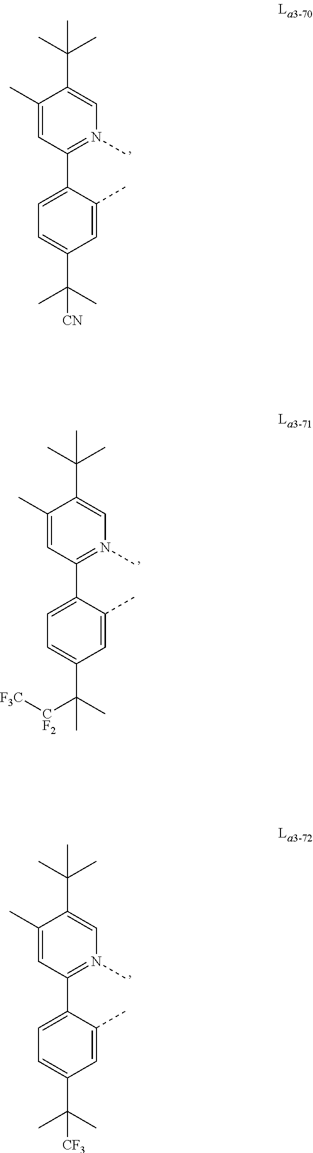 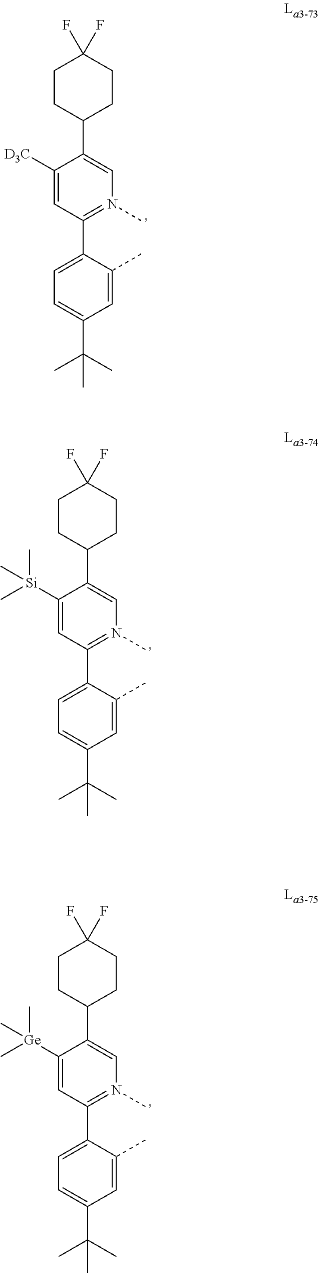 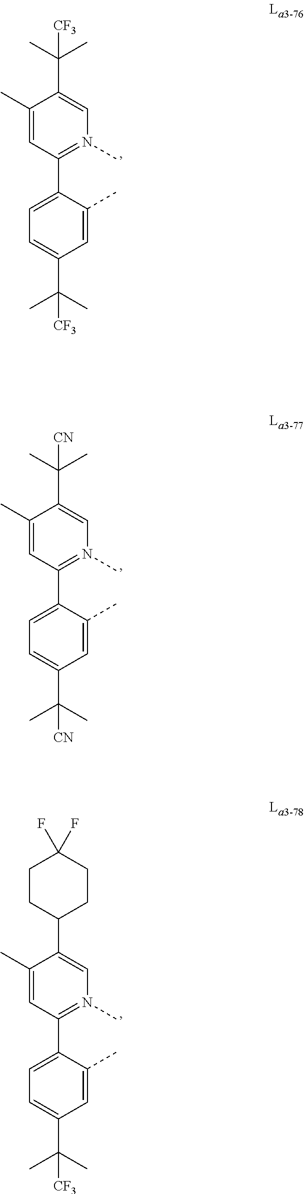 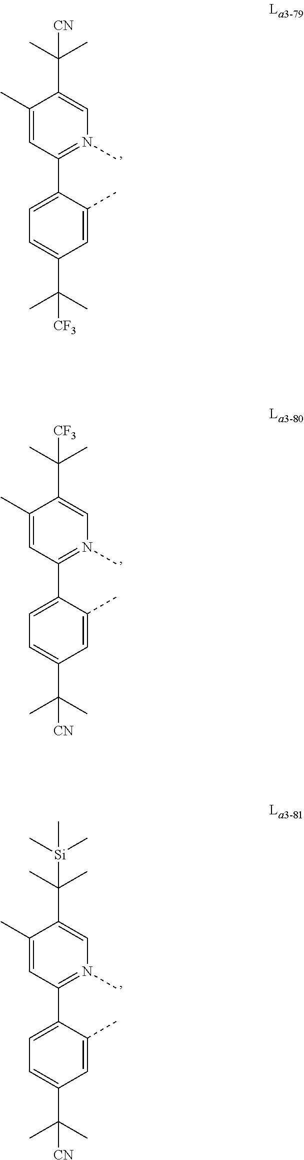 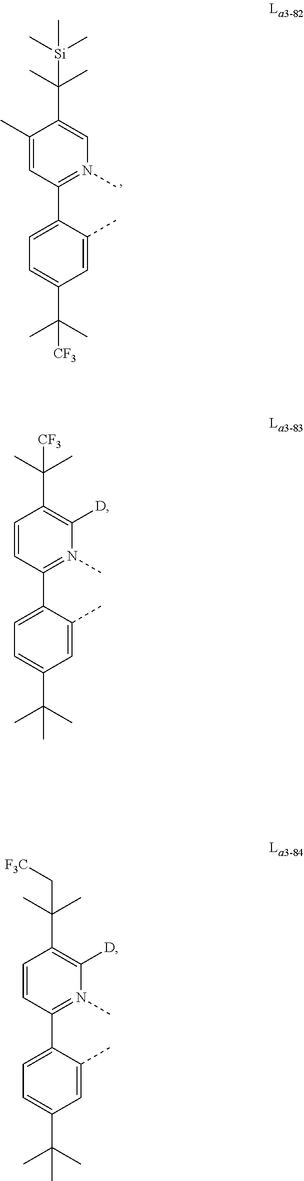 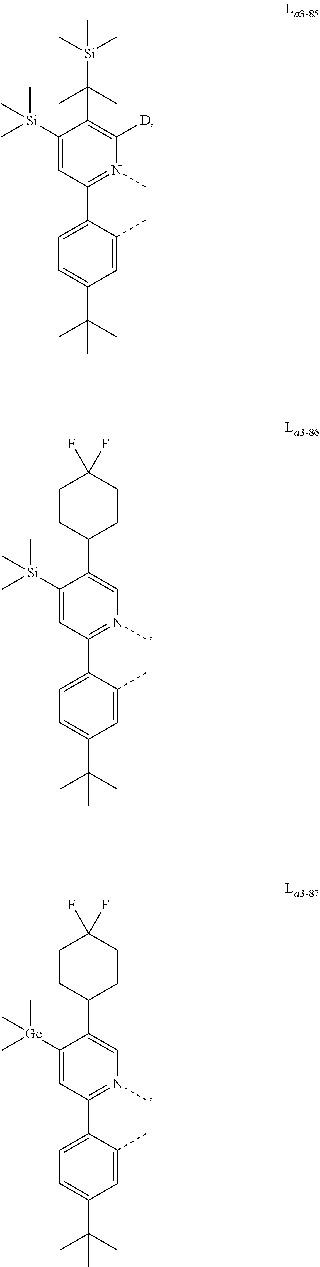 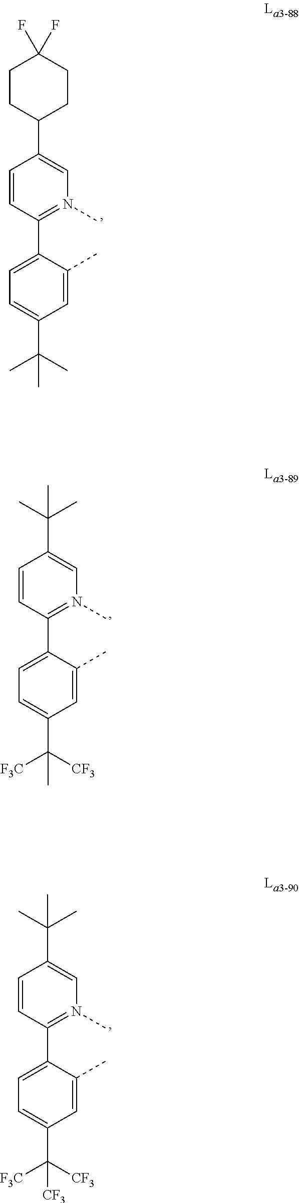 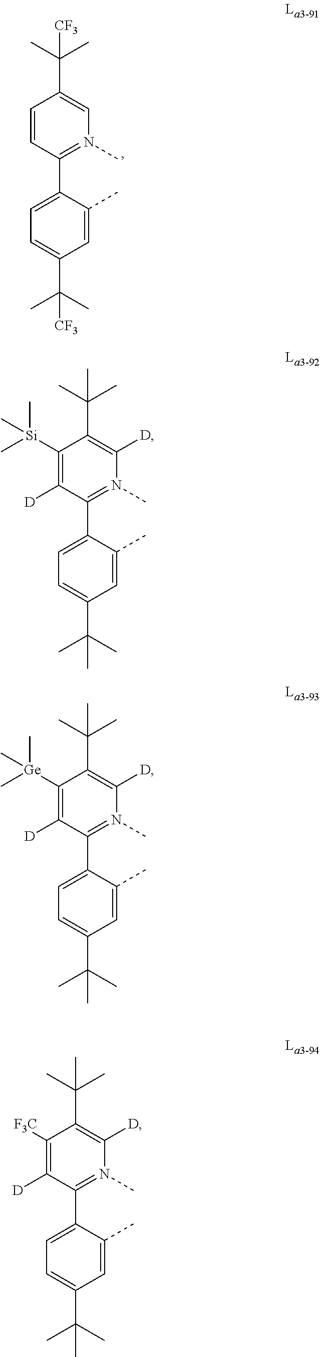 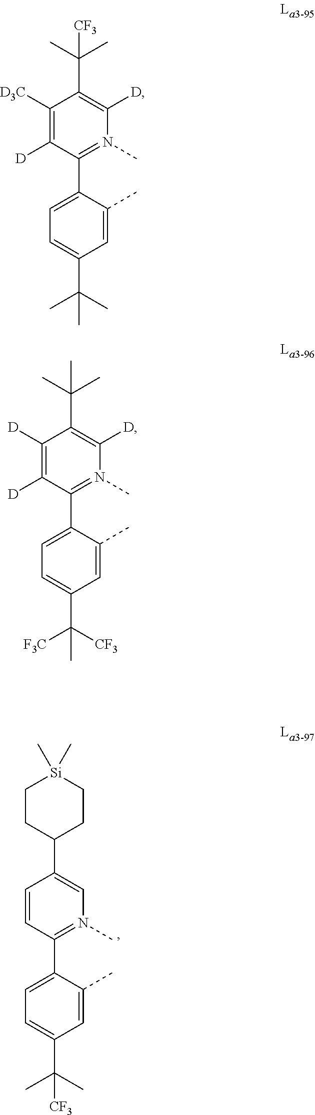 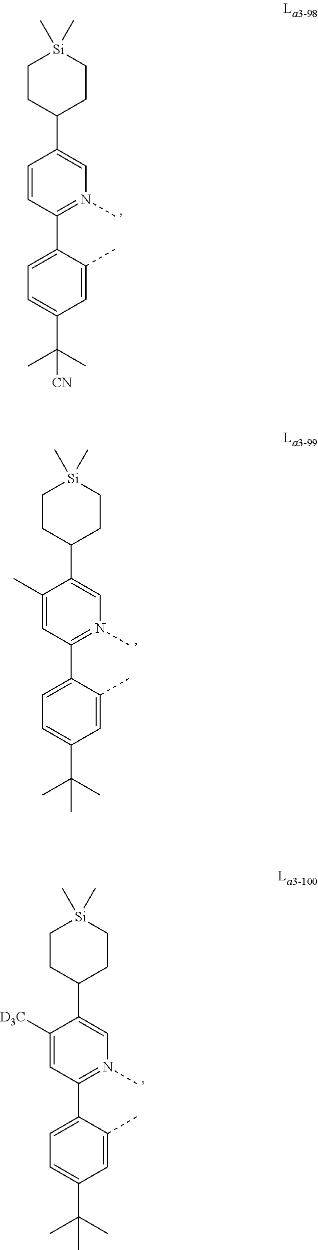 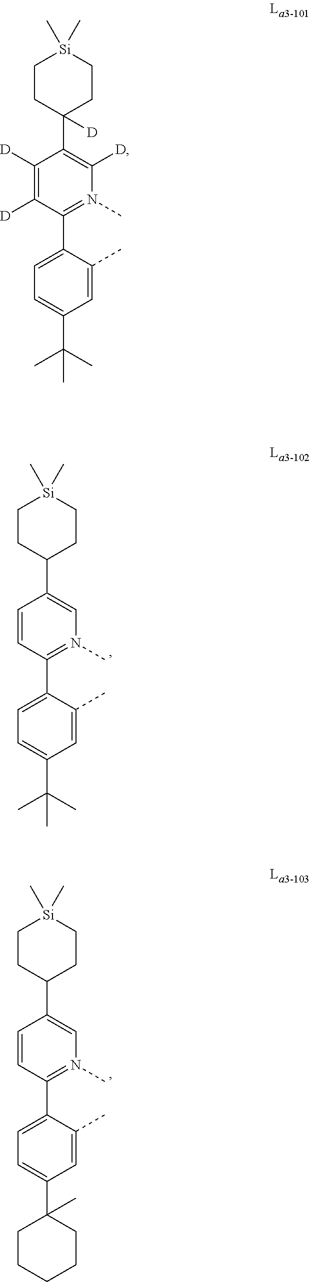 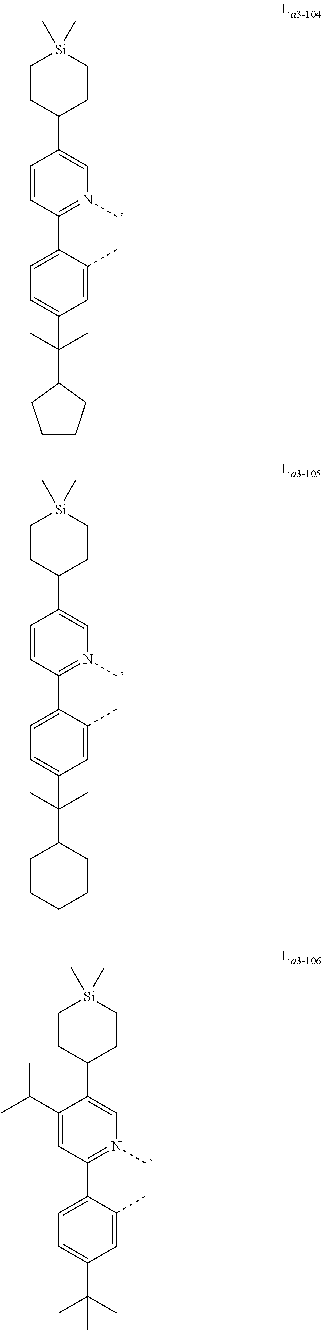 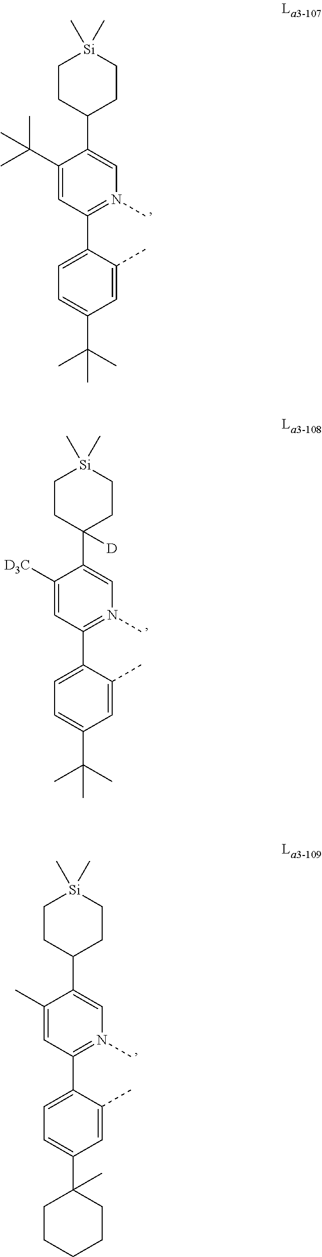 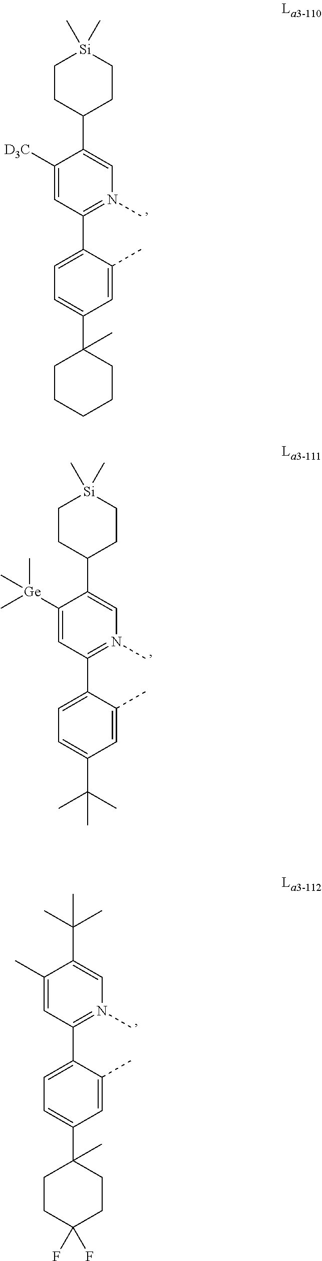 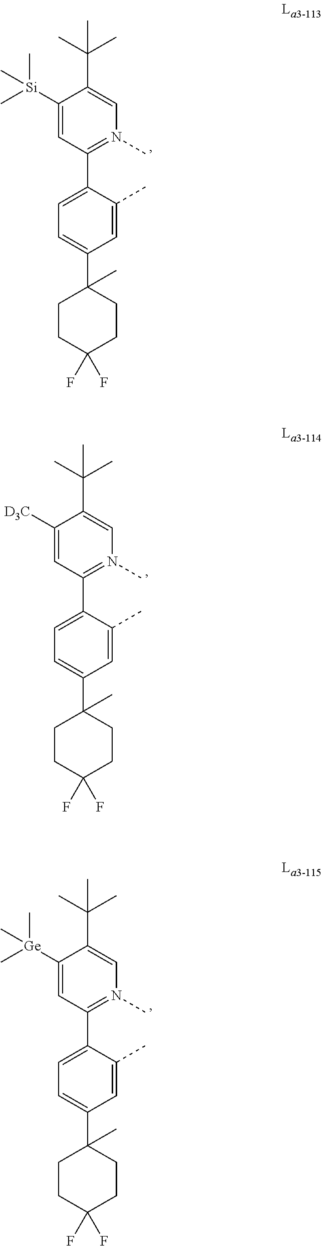 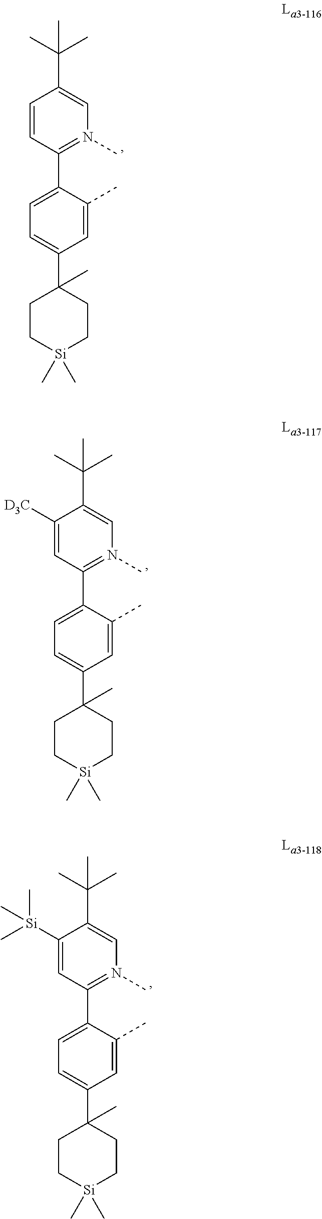 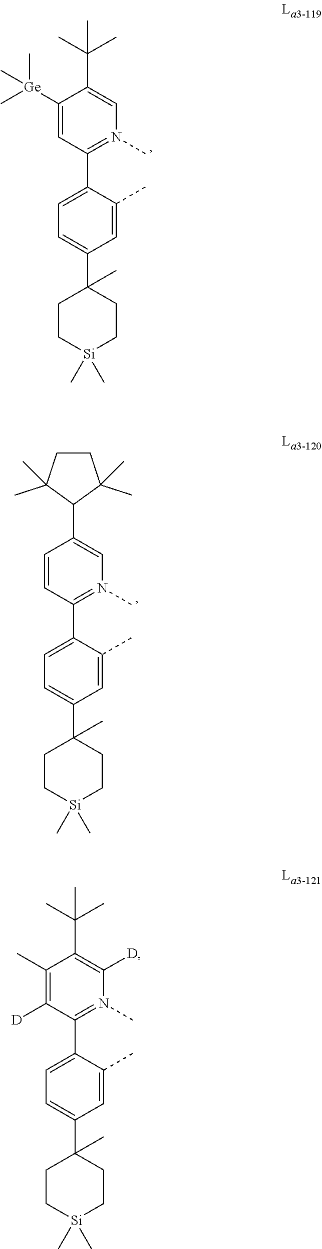 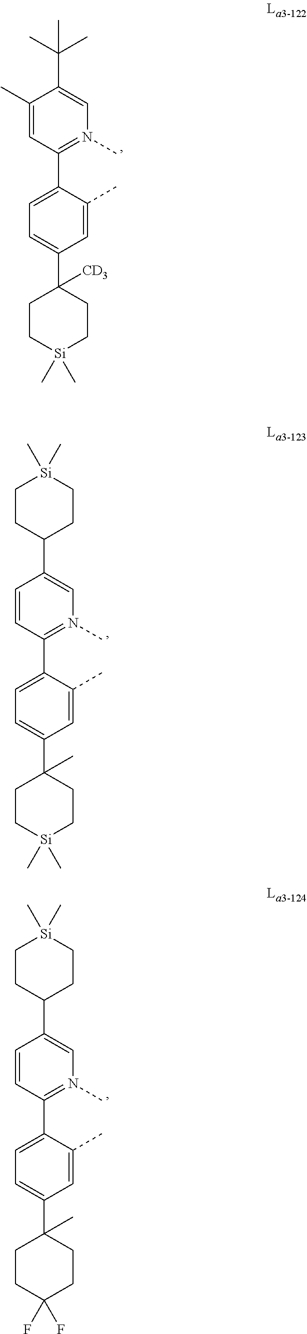 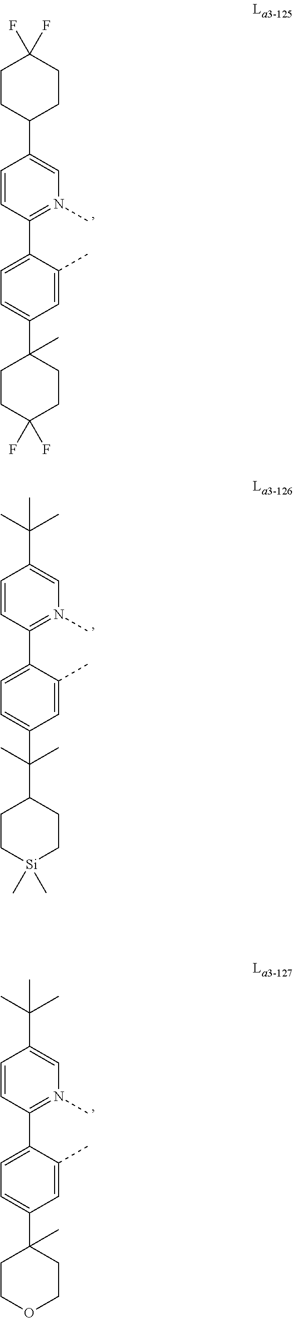 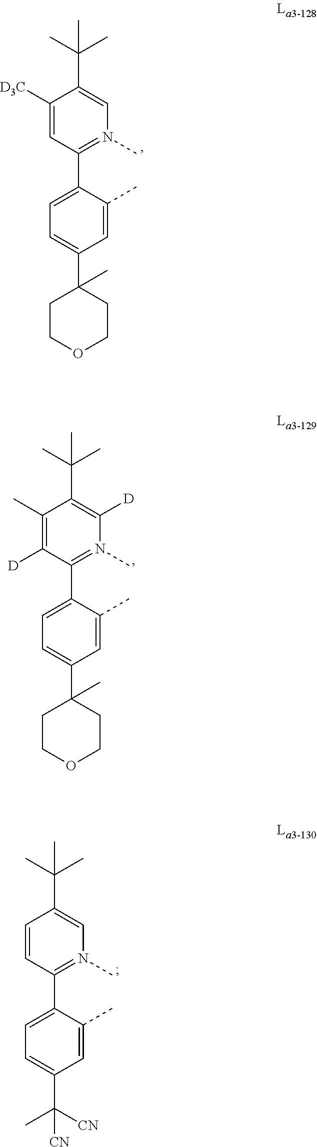
wherein optionally, hydrogen atoms in Lₐ₁₋₁ to Lₐ₁₋₂₃₁, Lₐ₂₋₁ to Lₐ₂₋₁₆₁ and Lₐ₃₋₁ to Lₐ₃₋₁₃₀ can be partially or fully substituted with deuterium atoms.

18. The metal complex according to claim 1, wherein L_{b} is, at each occurrence identically or differently, selected from the group consisting of the following: 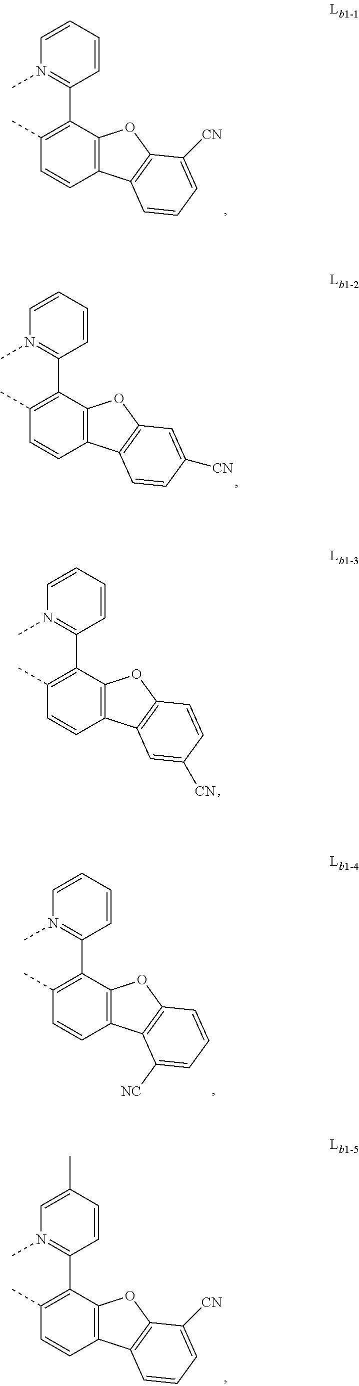 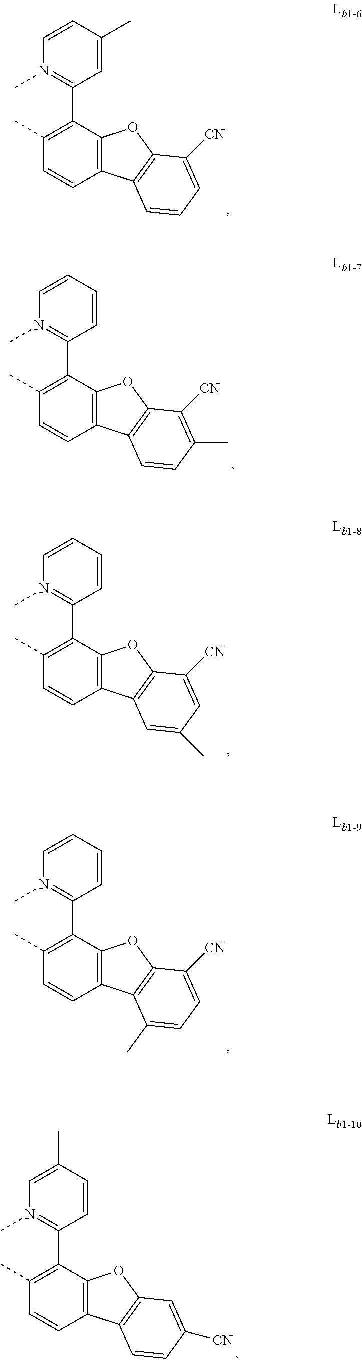 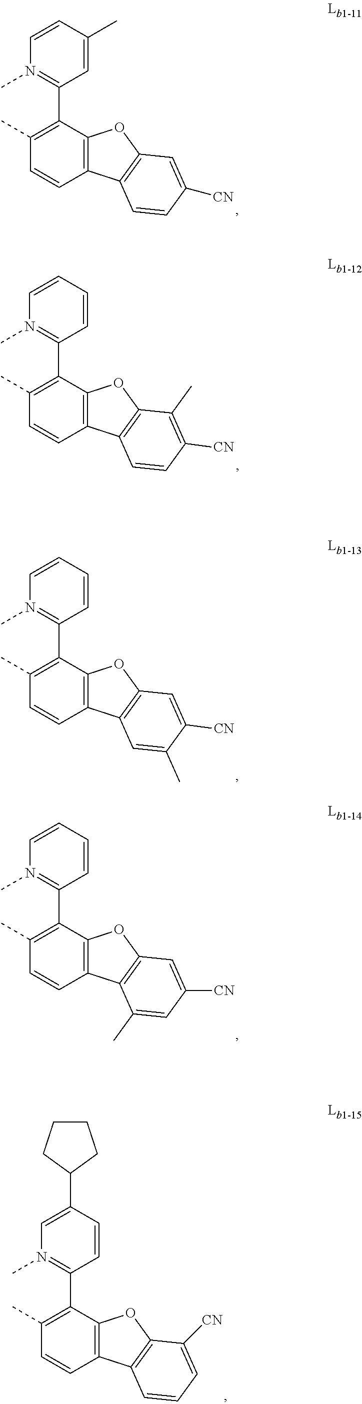 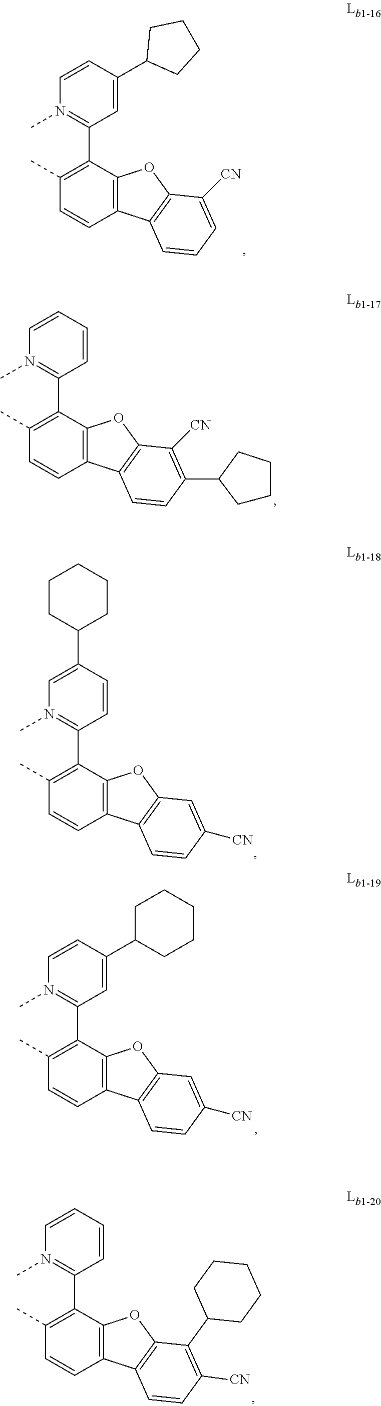 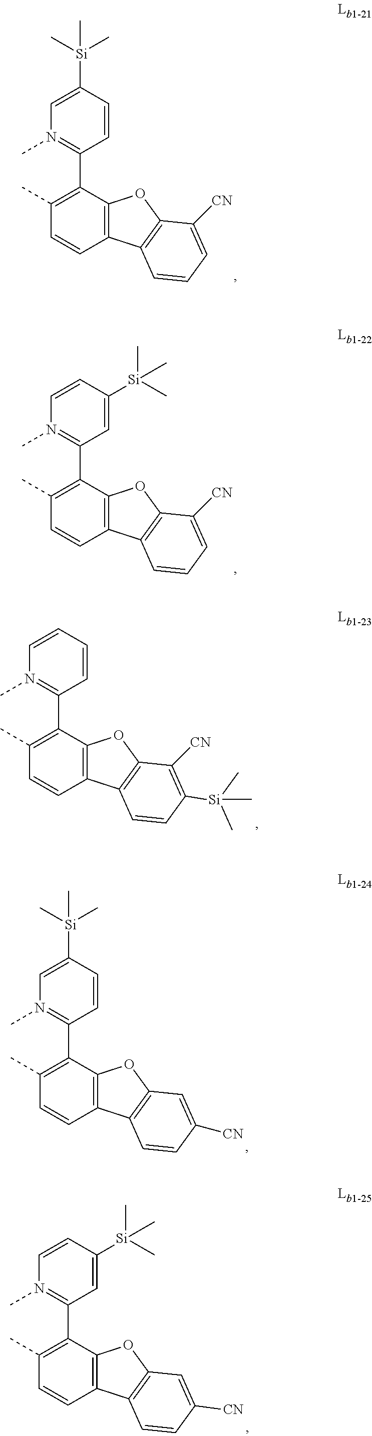 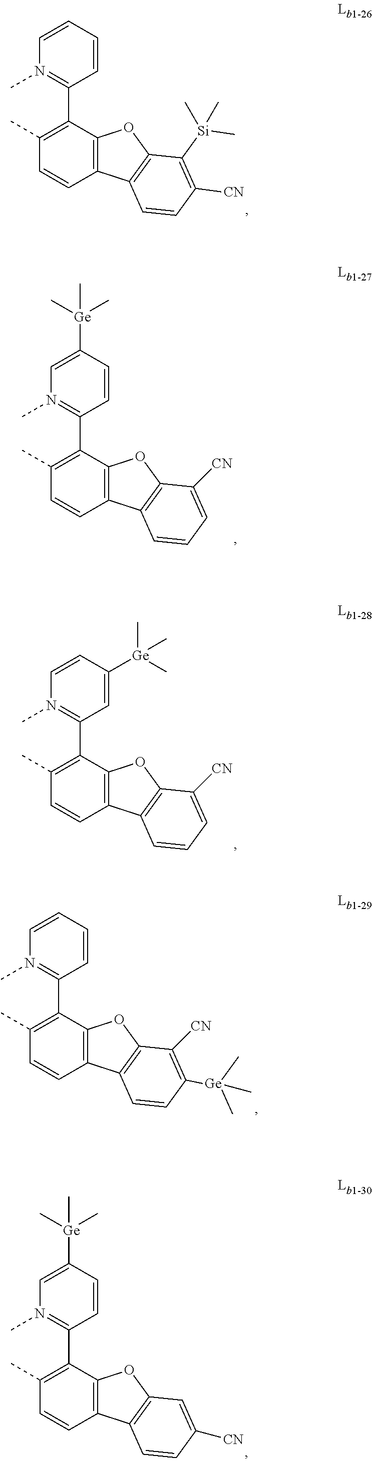 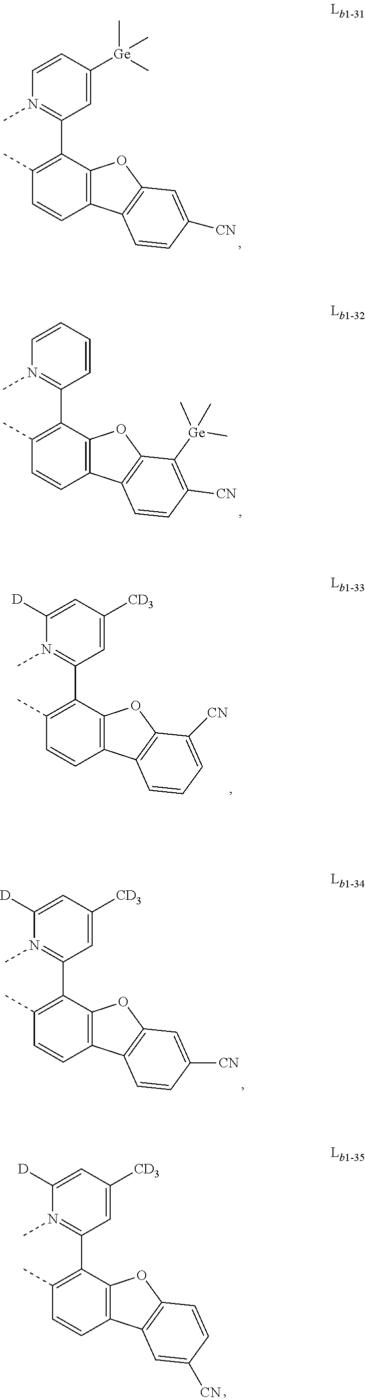 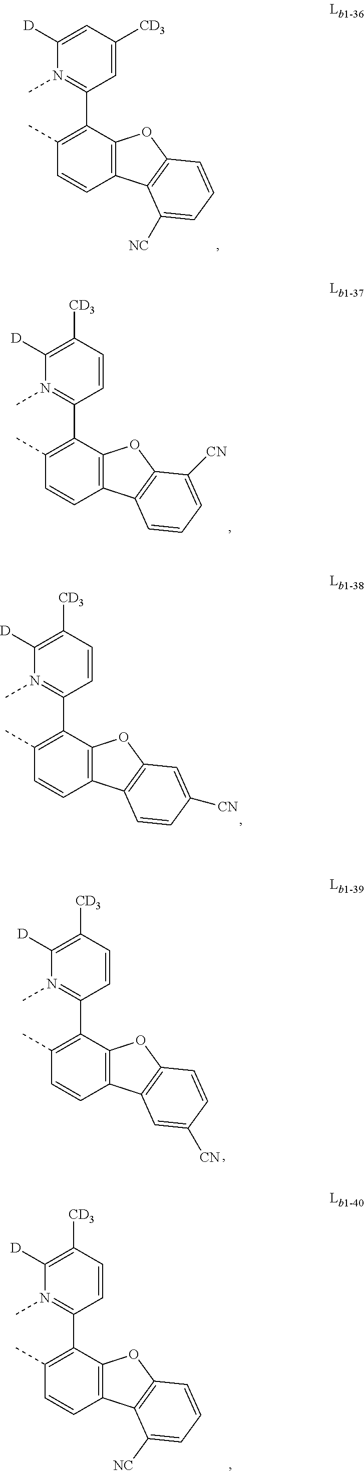 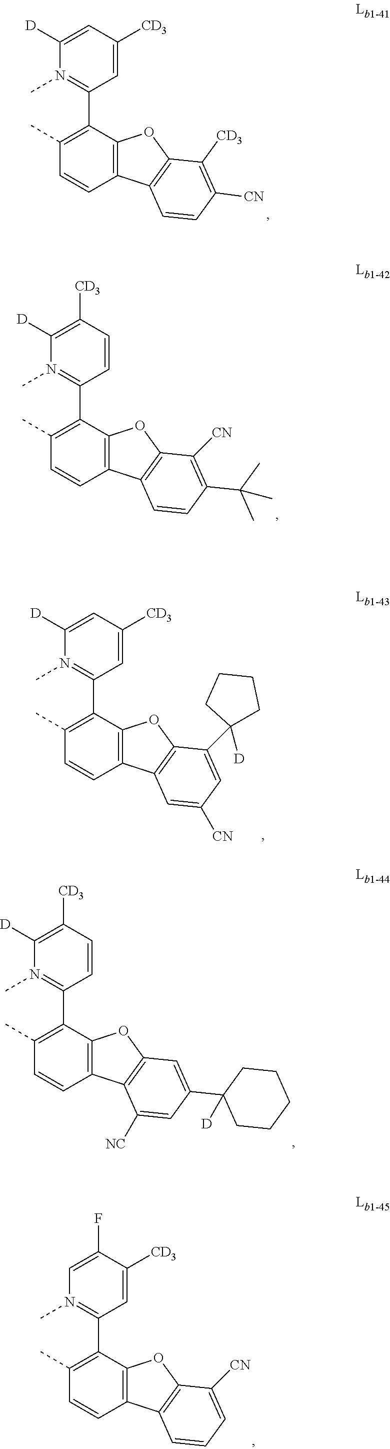 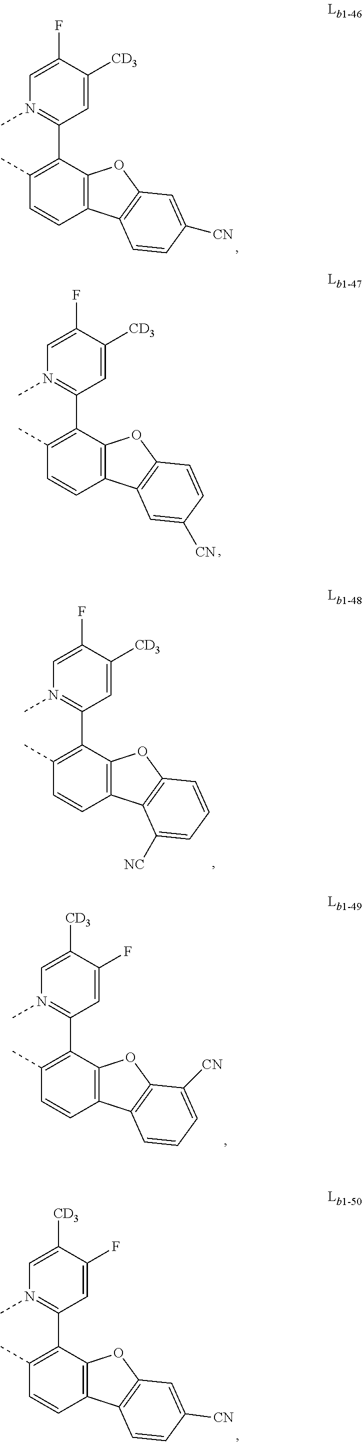 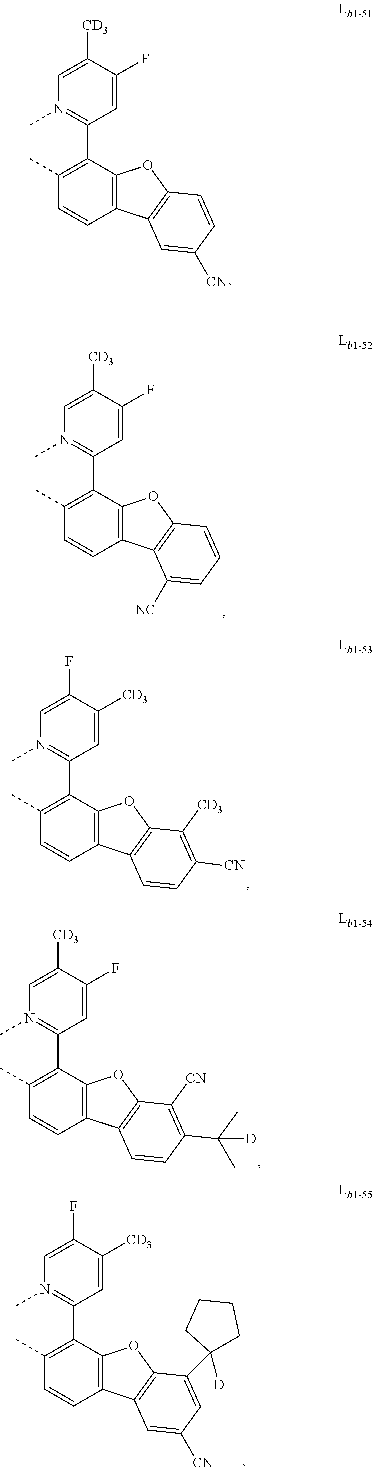 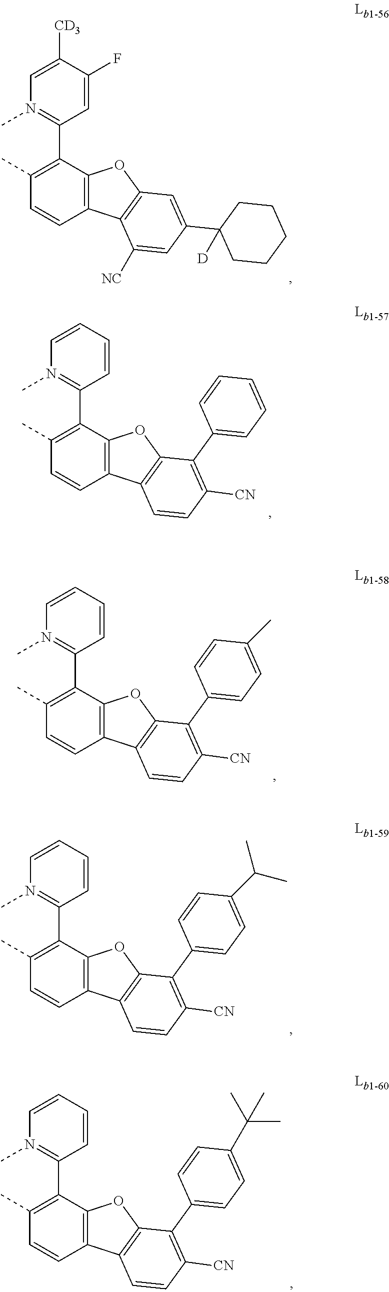 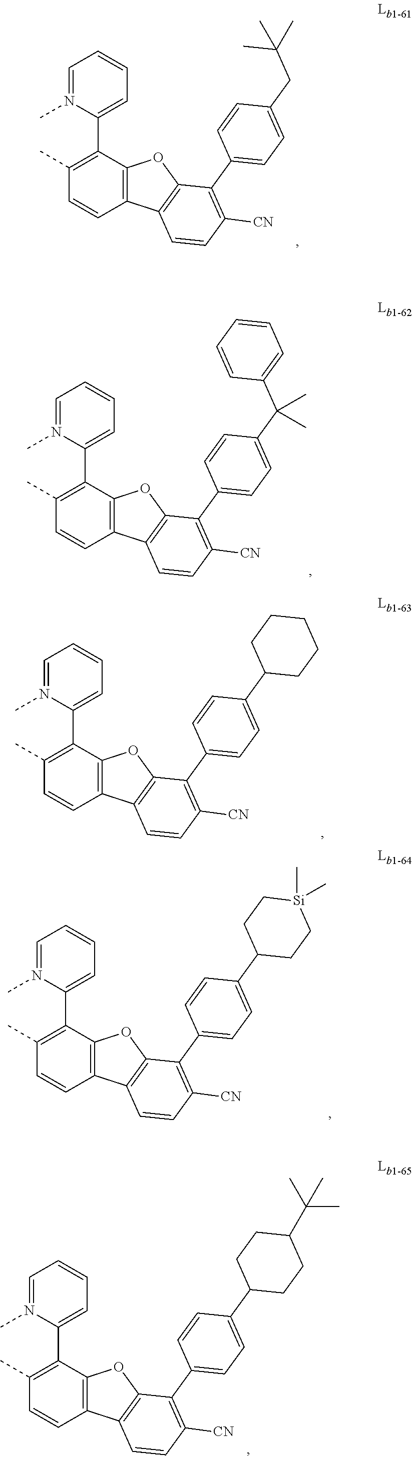 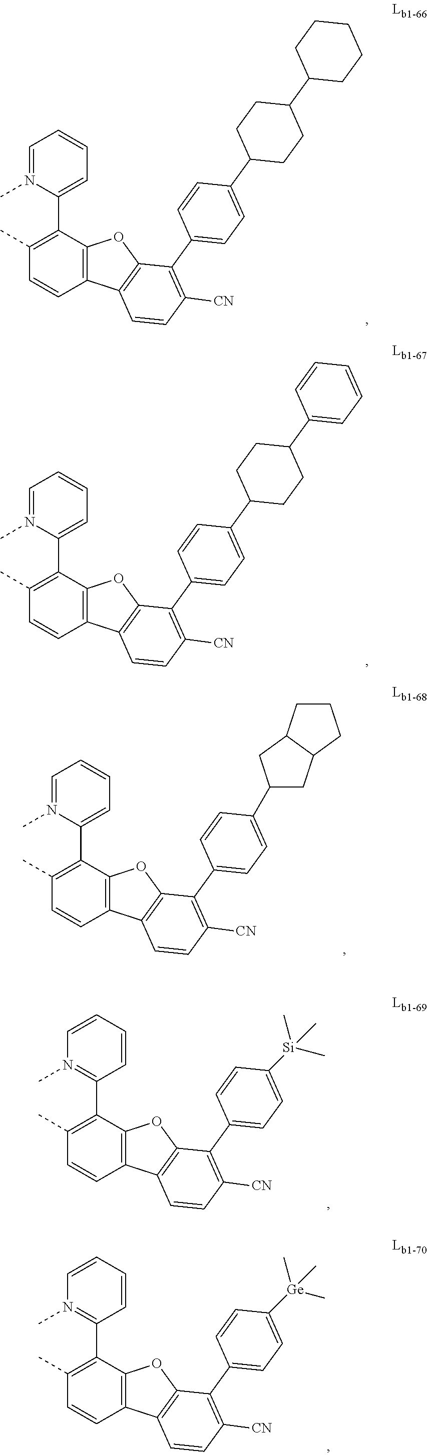 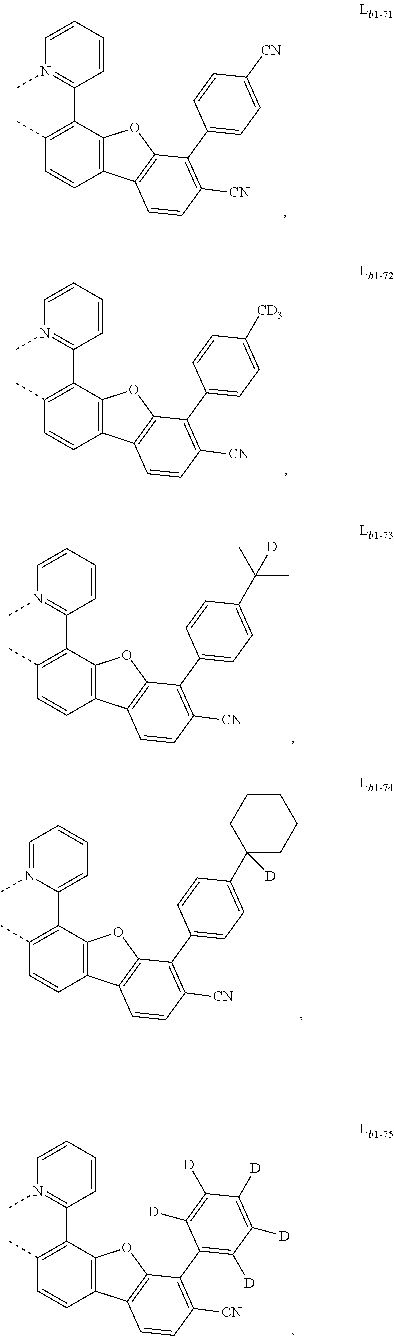 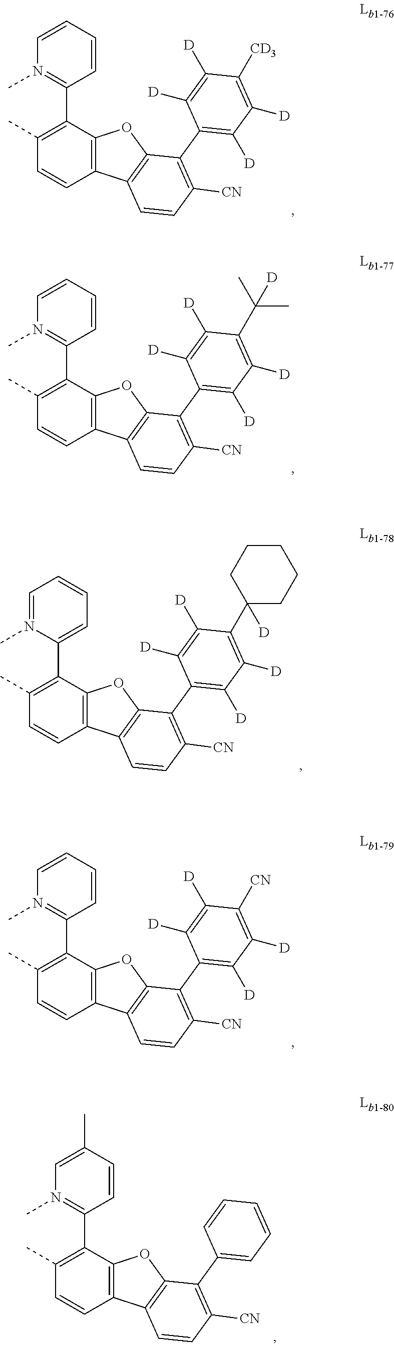 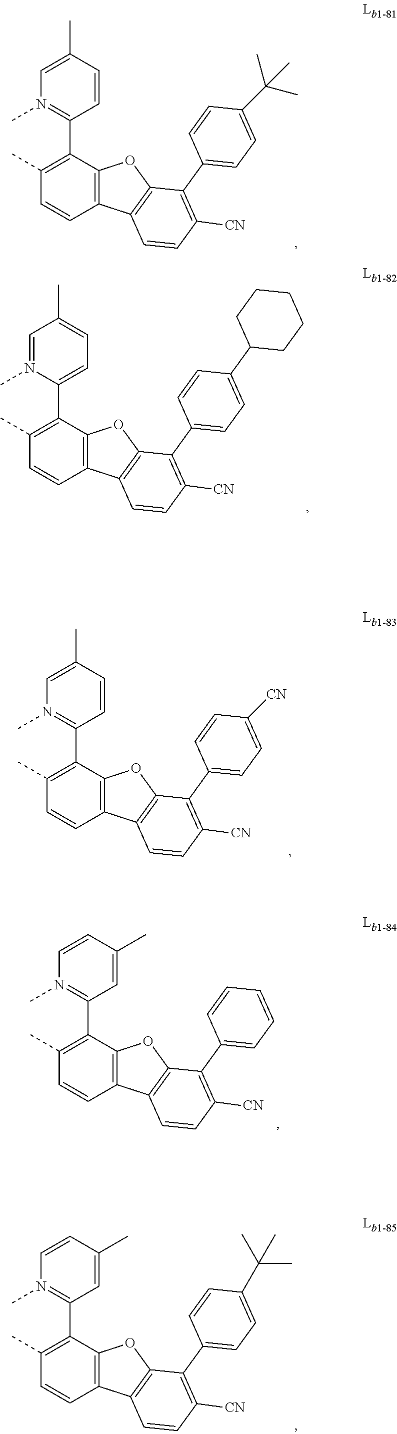 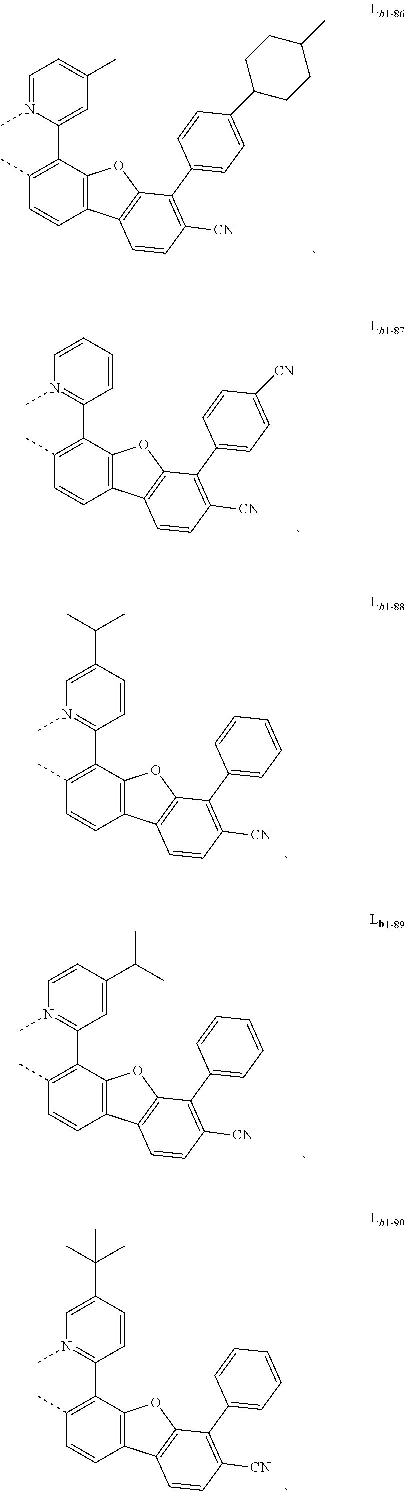 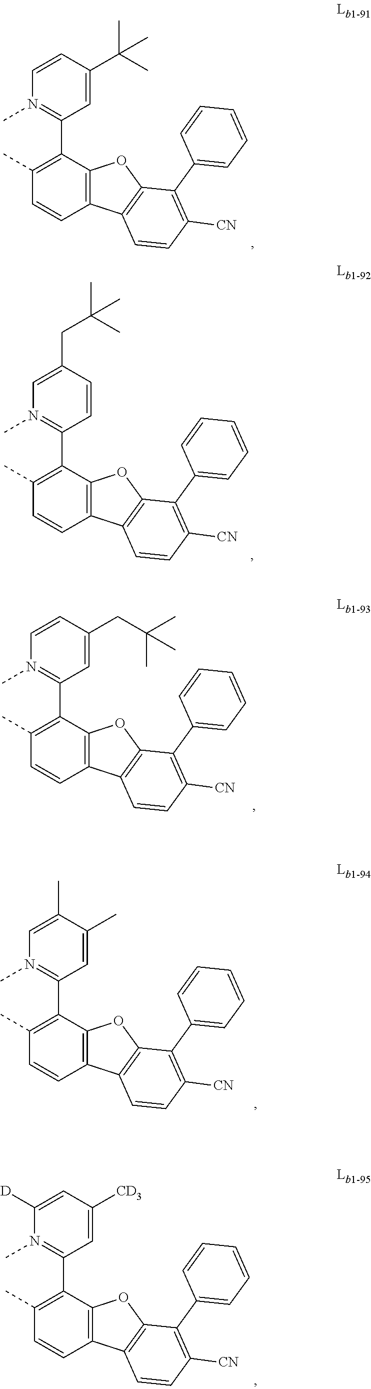 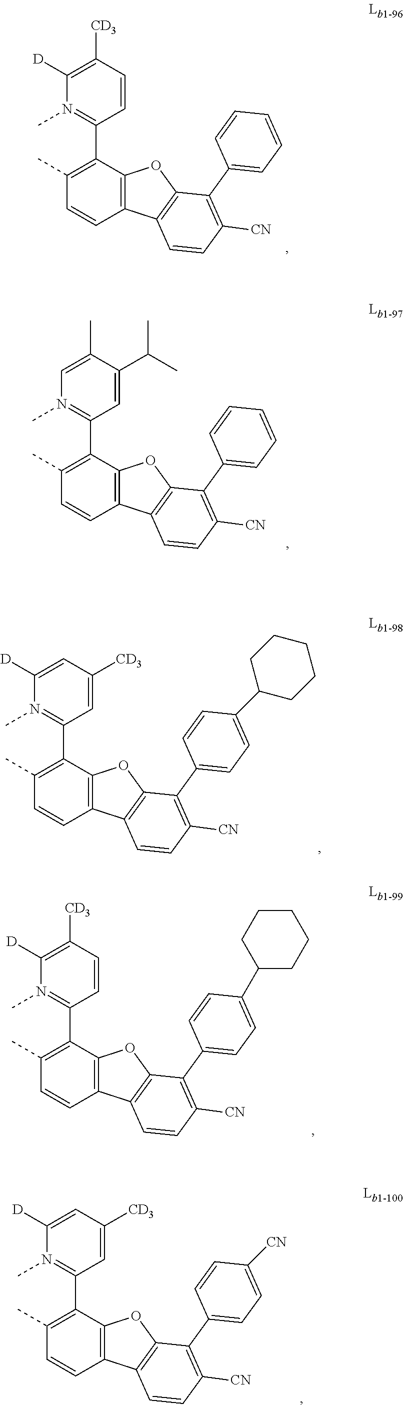 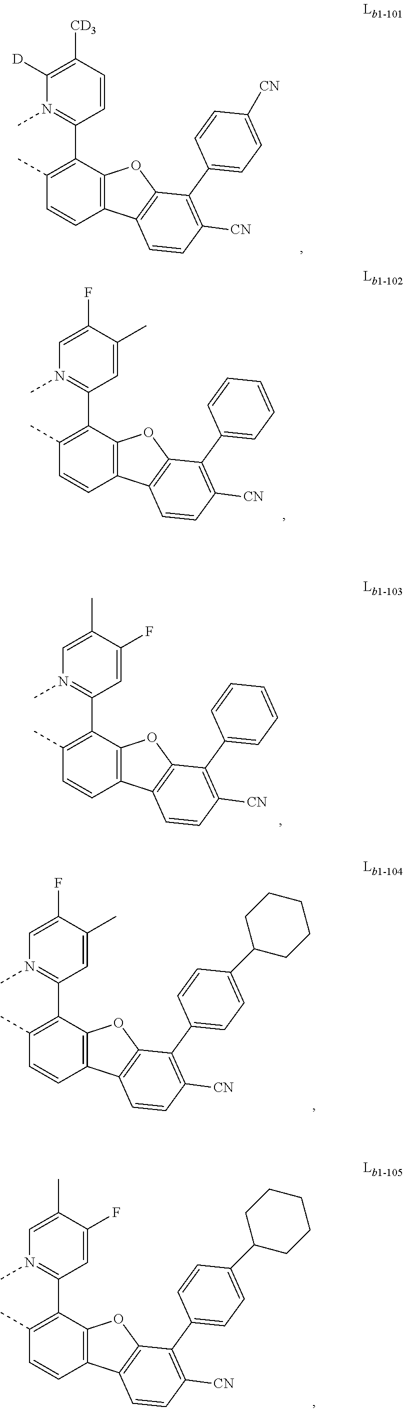 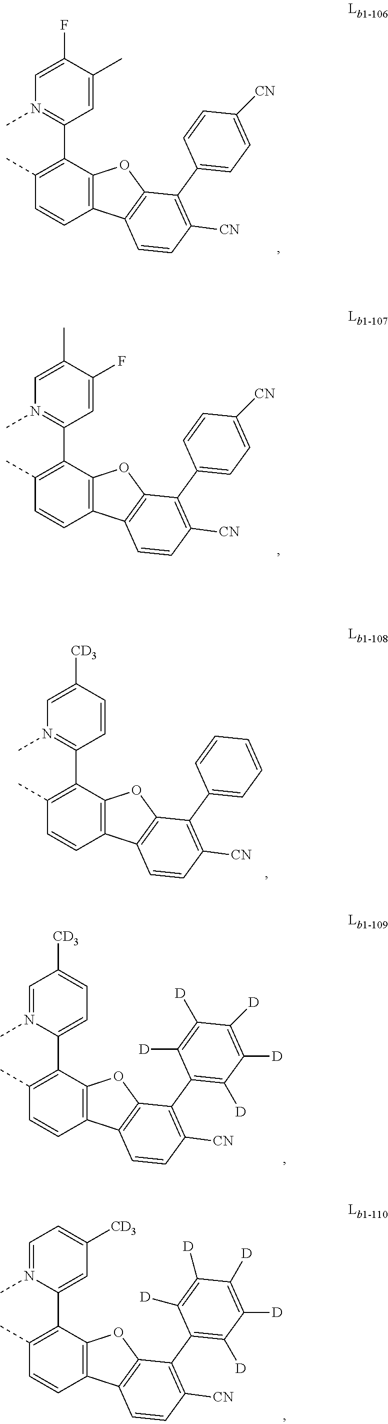 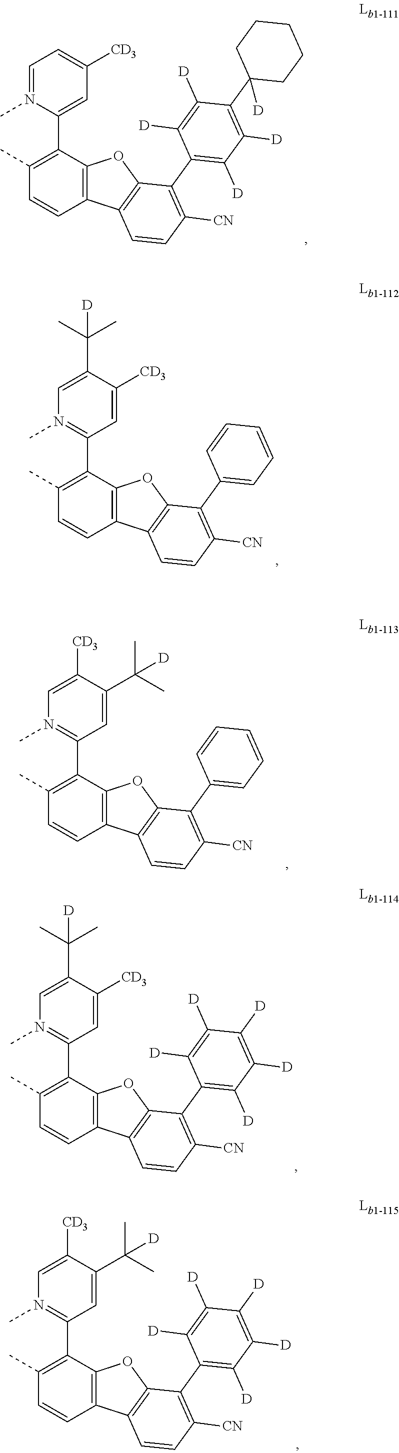 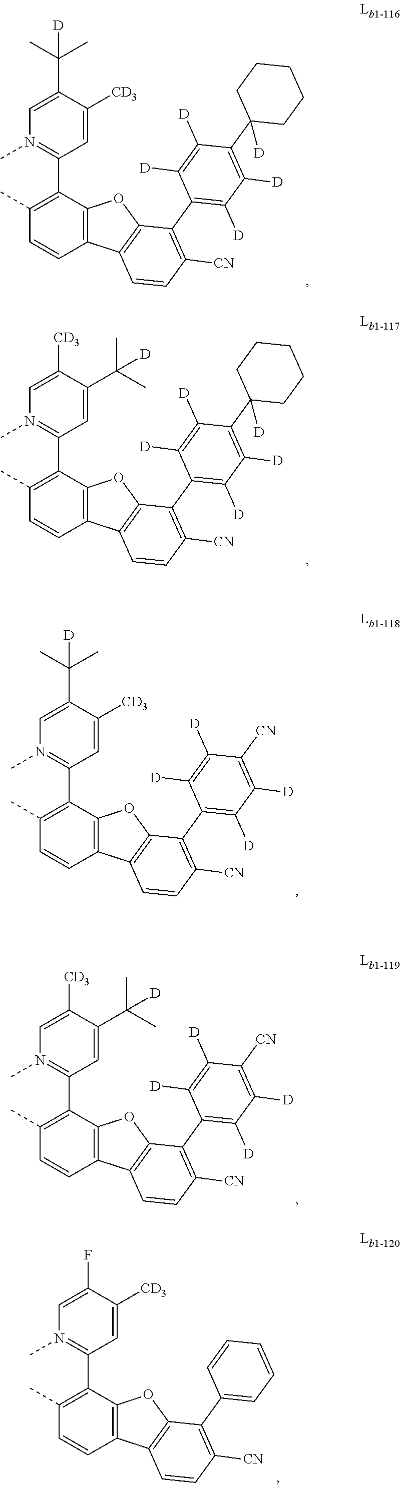 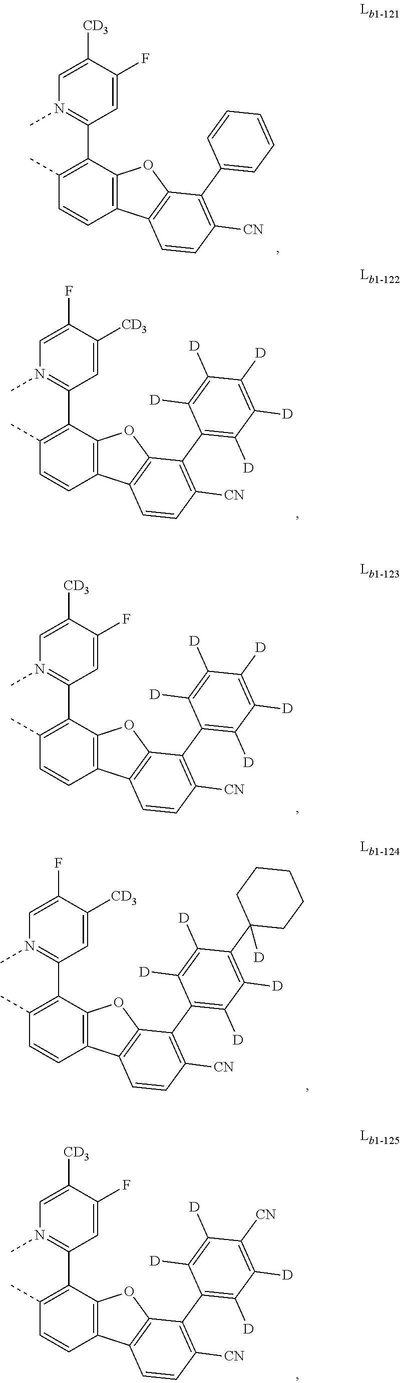 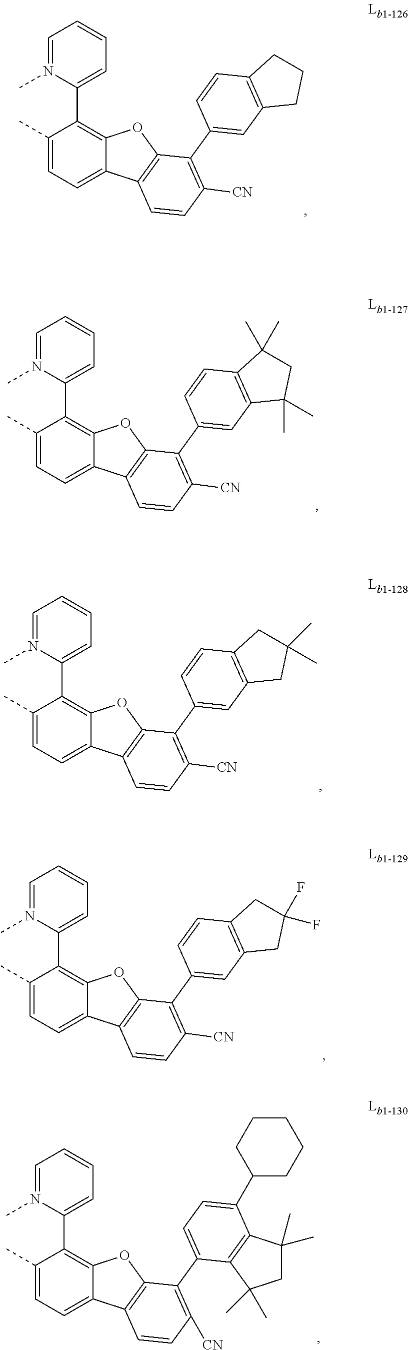 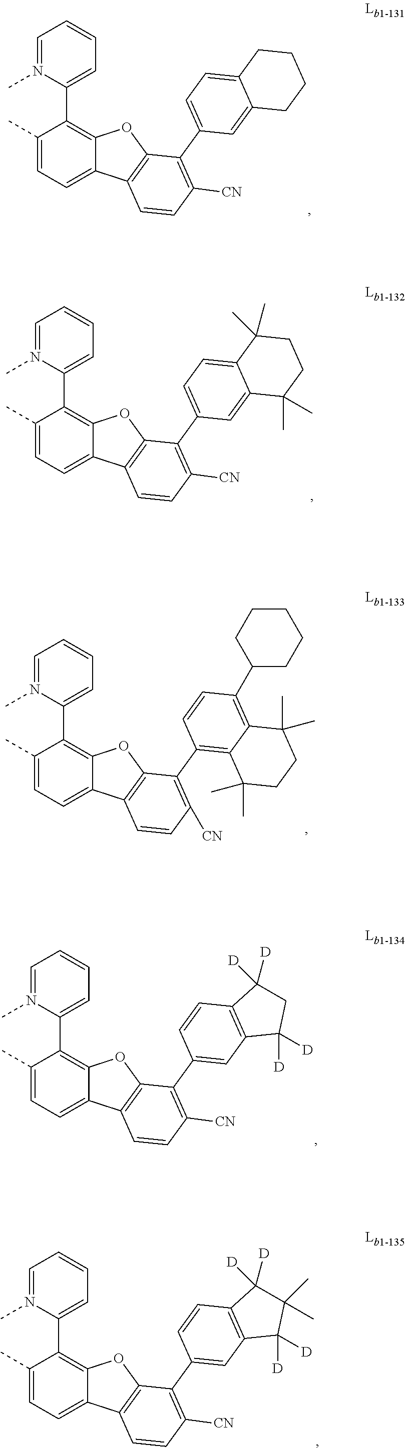 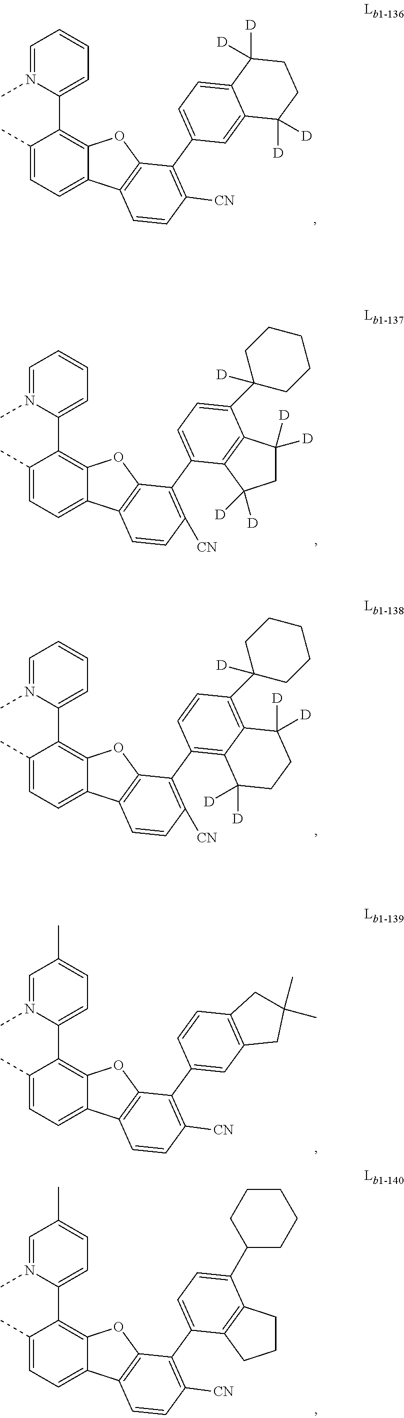 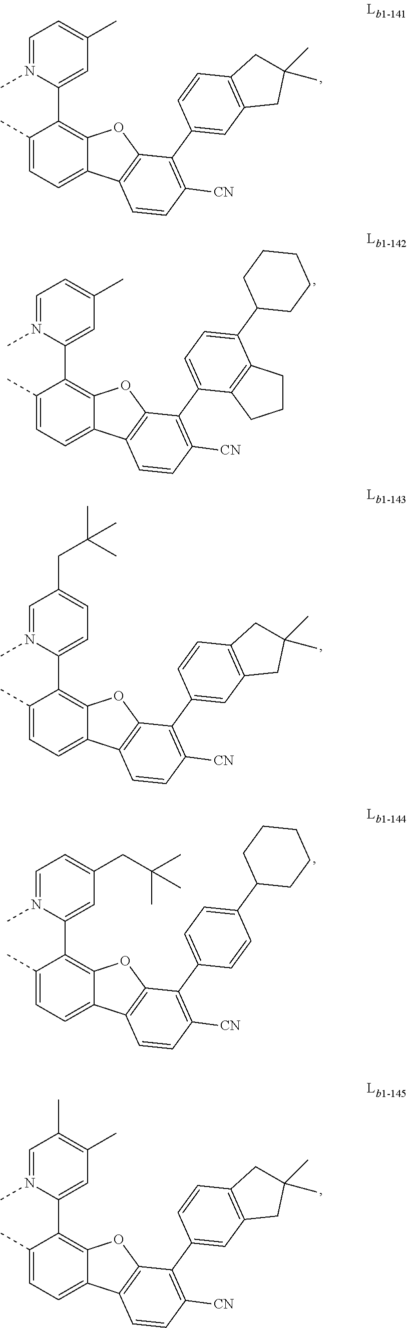 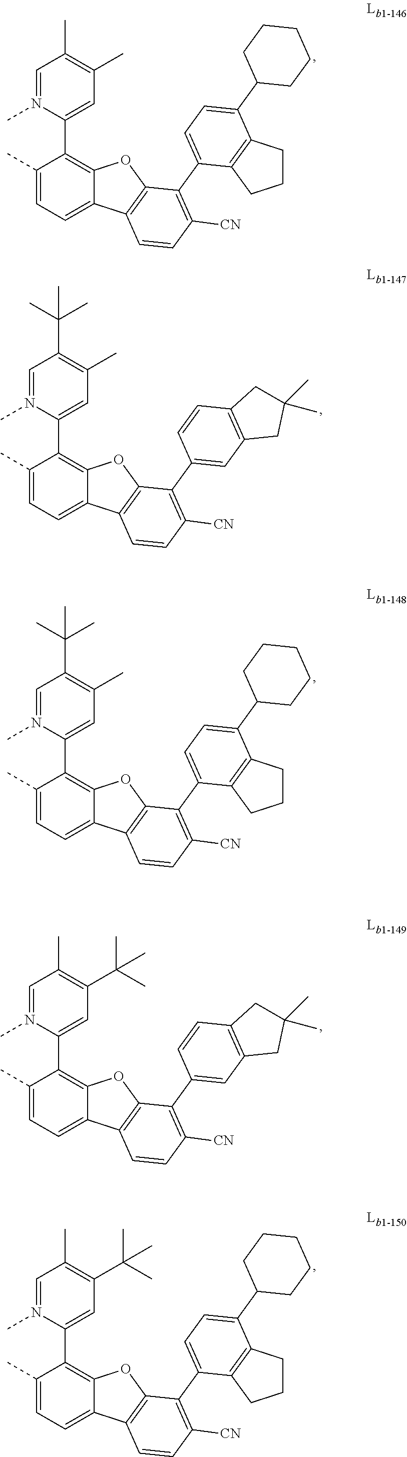 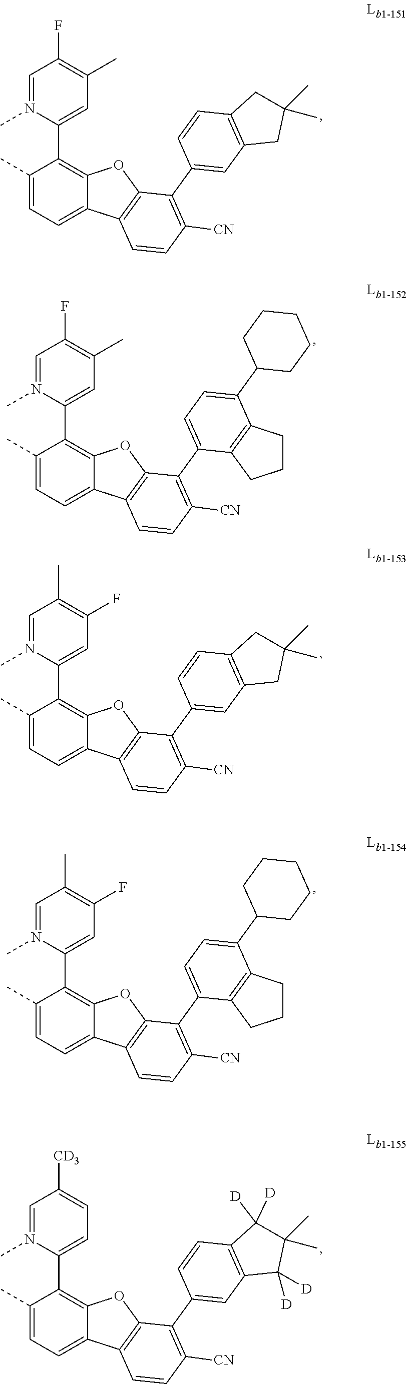 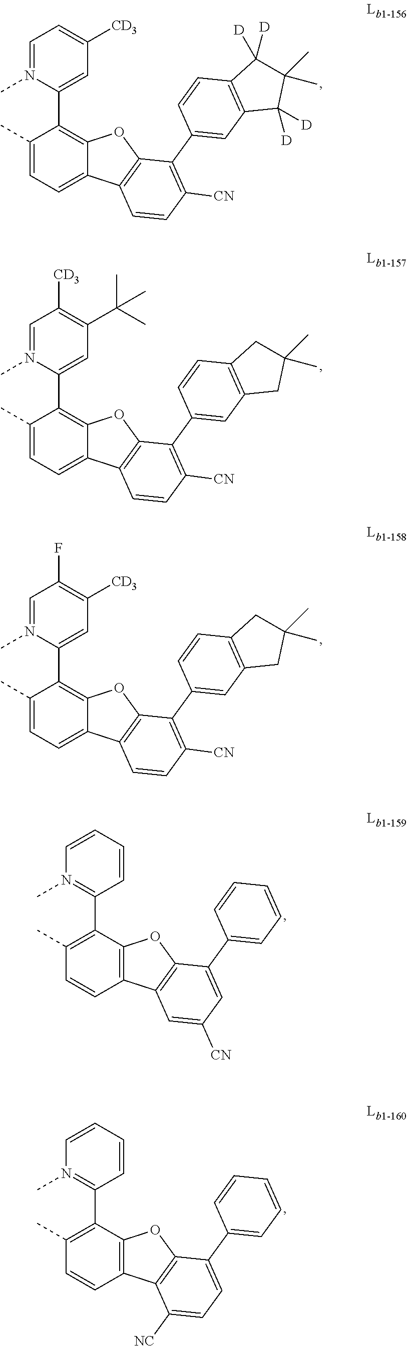 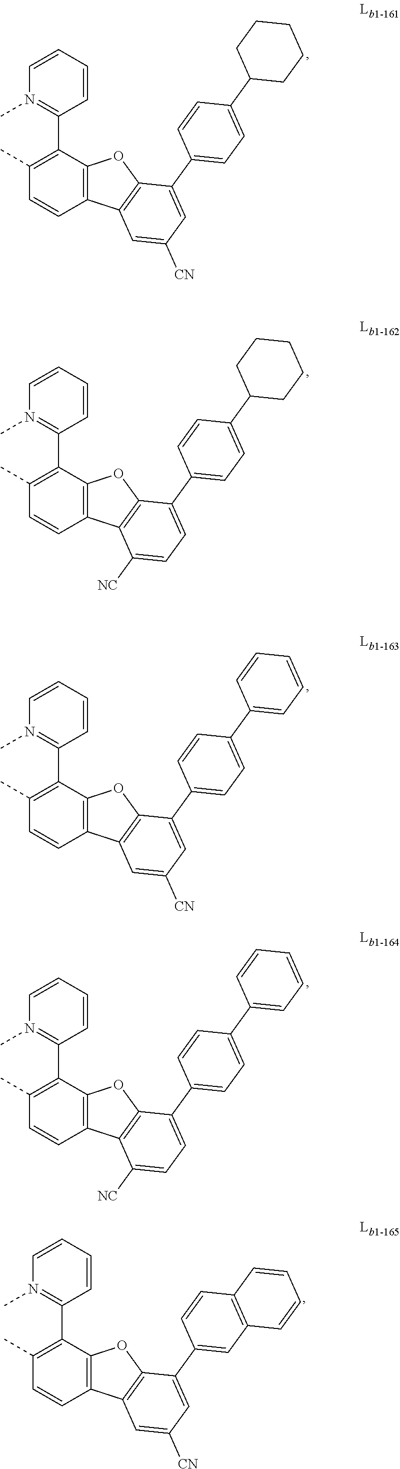 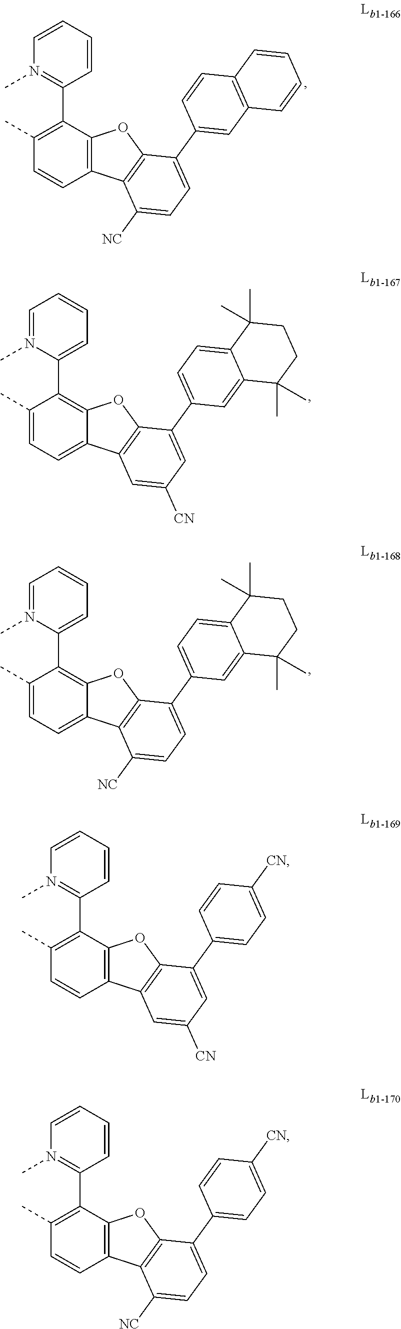 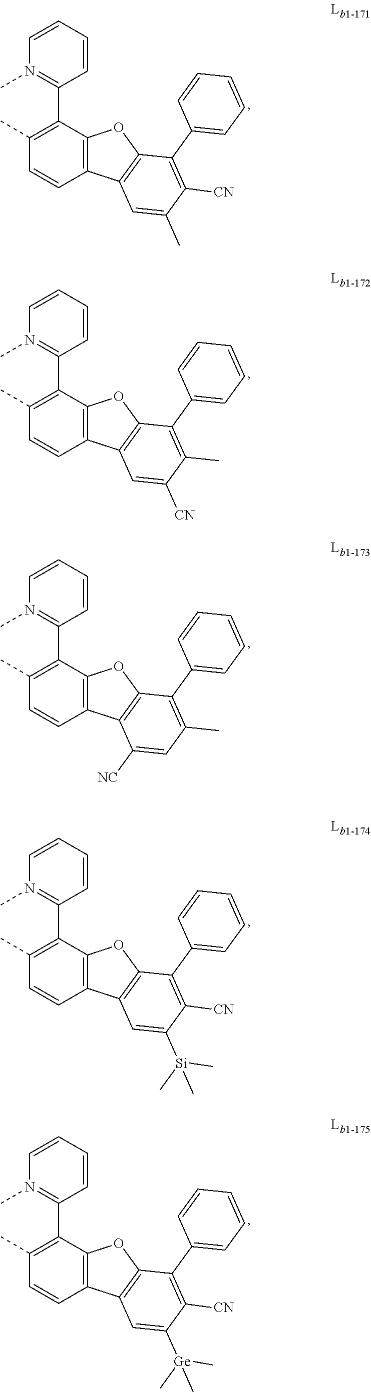 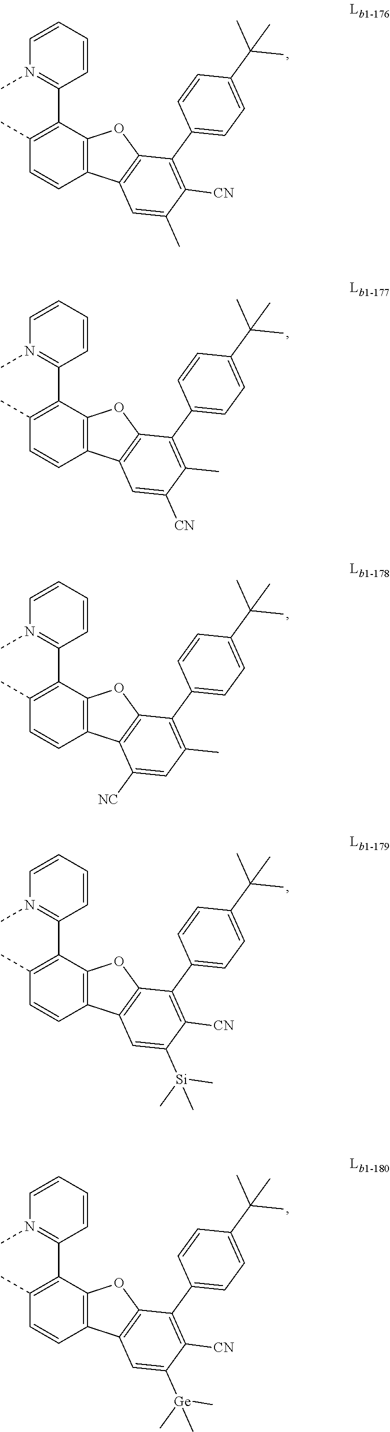 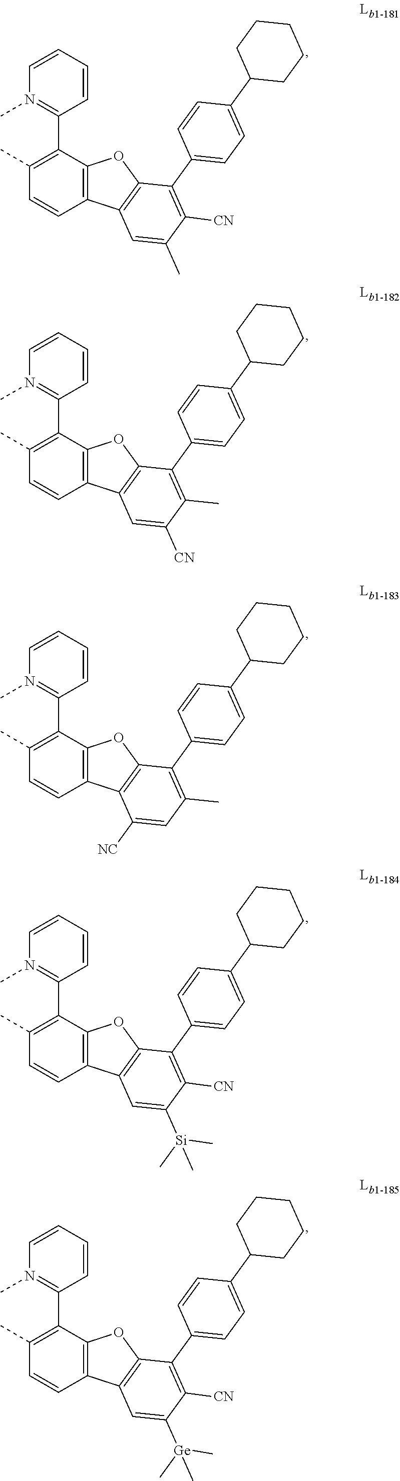 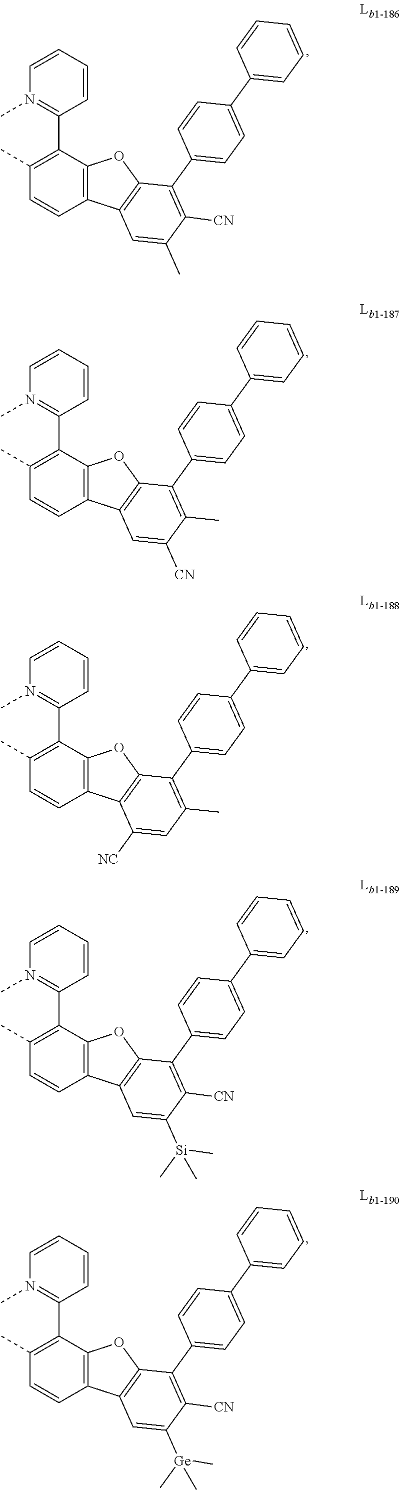 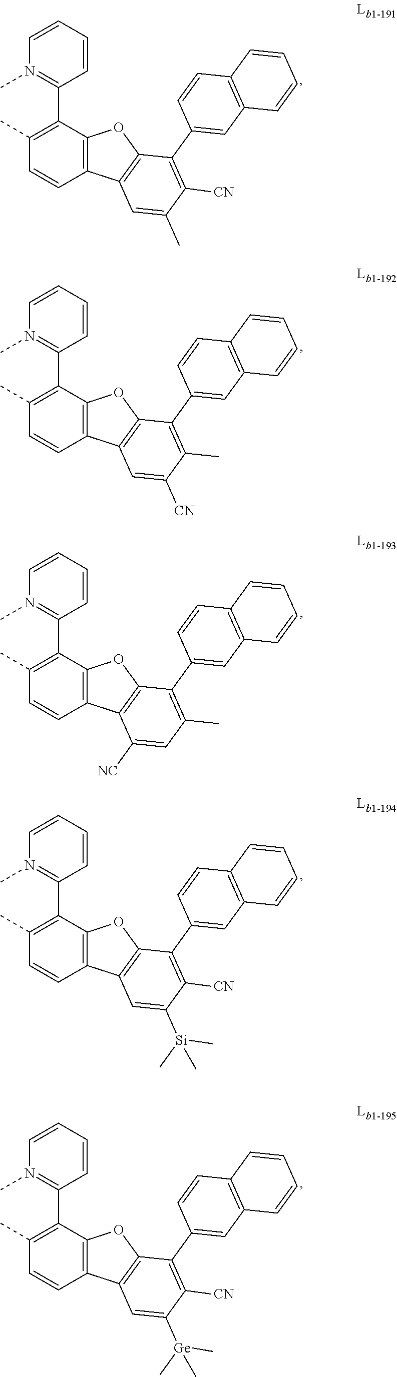 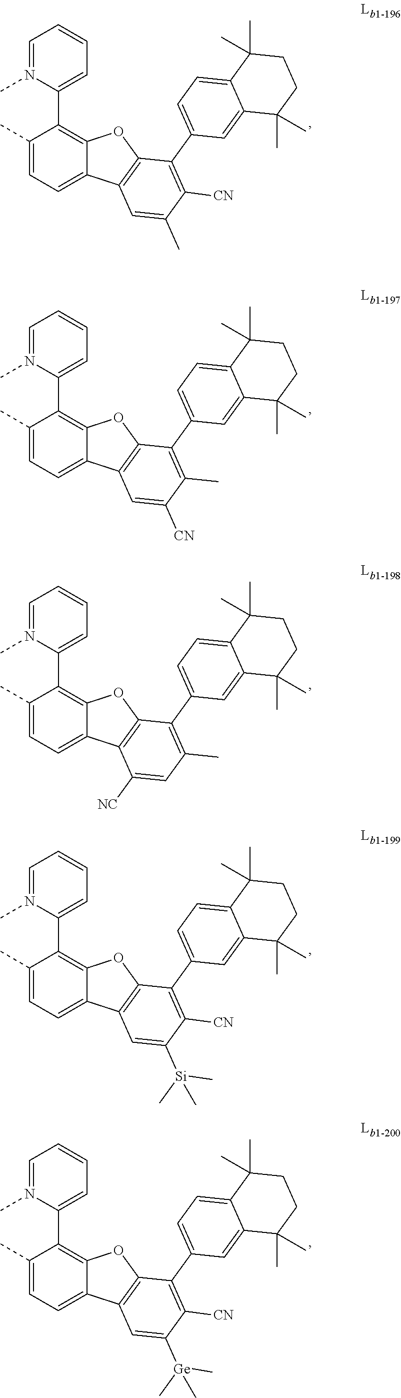 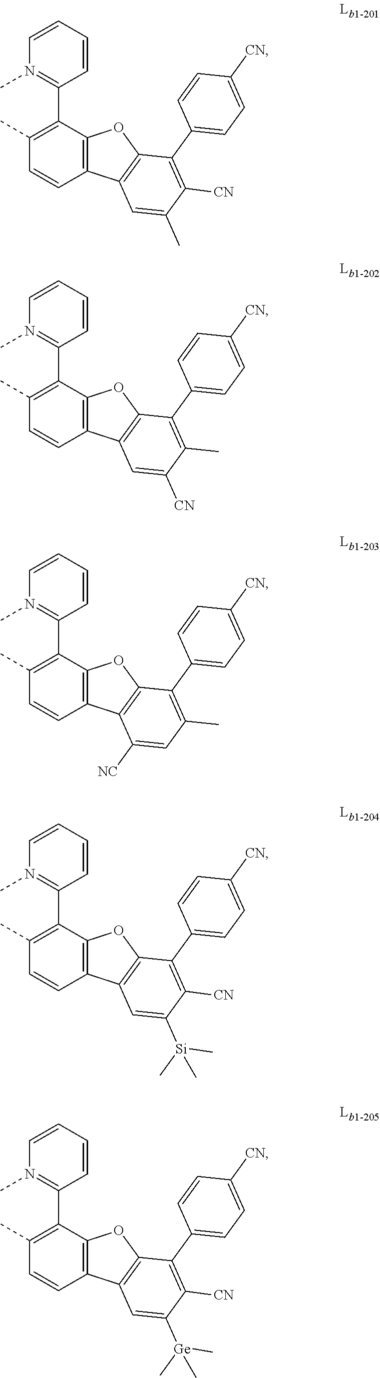 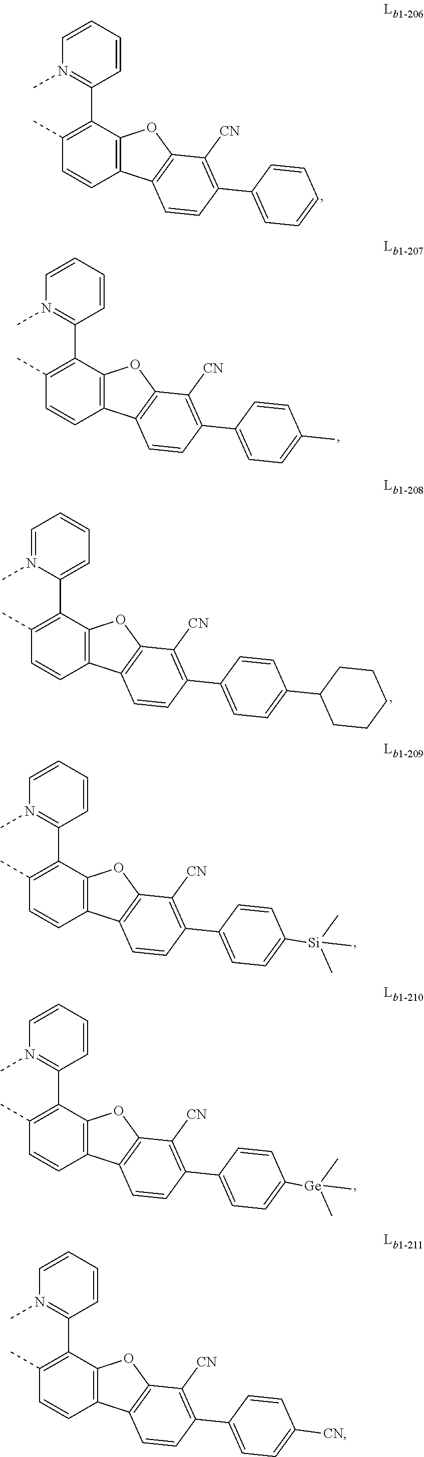 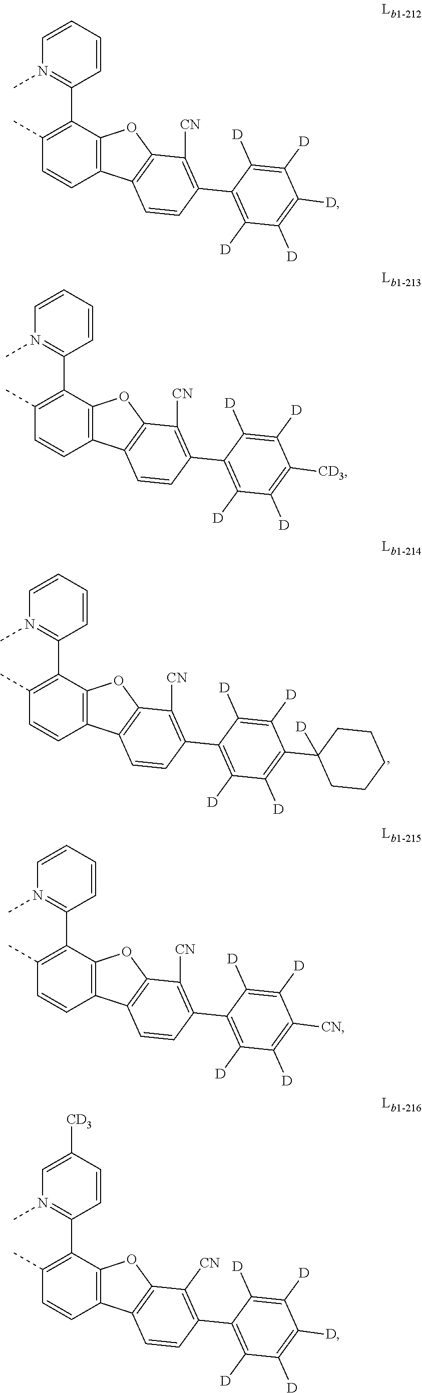 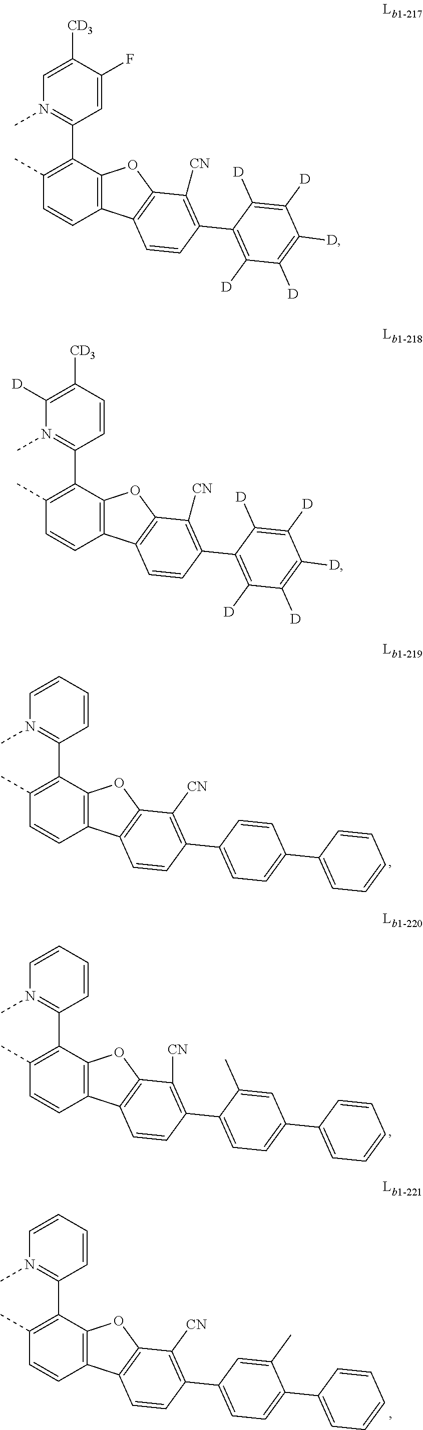 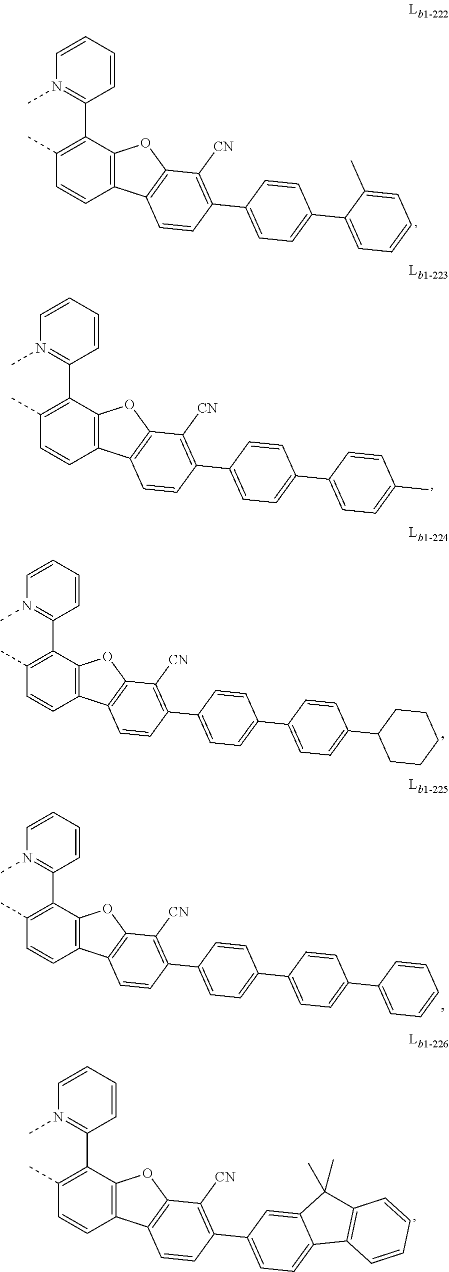 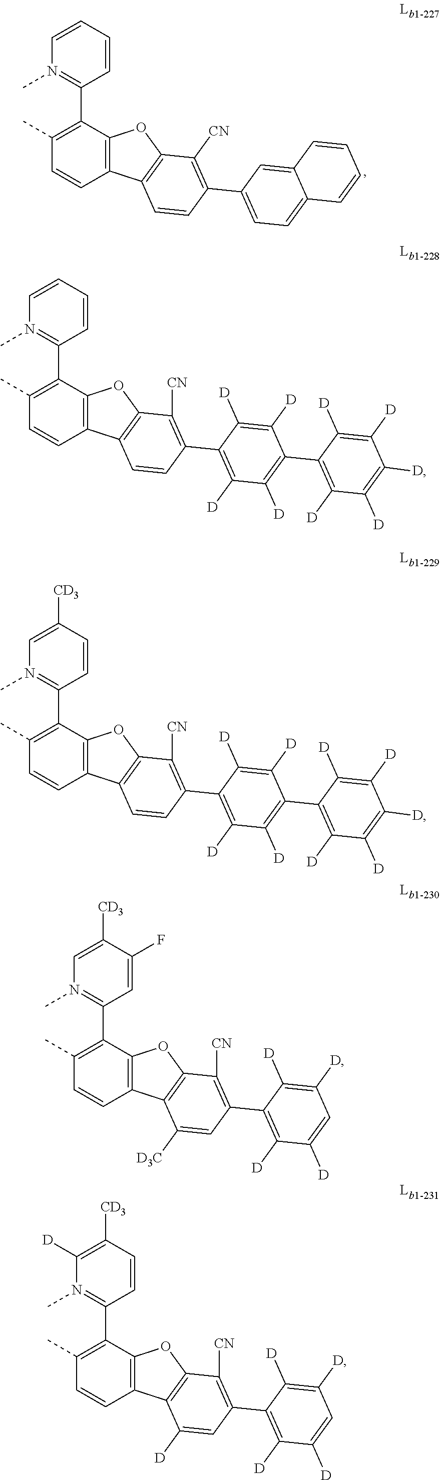 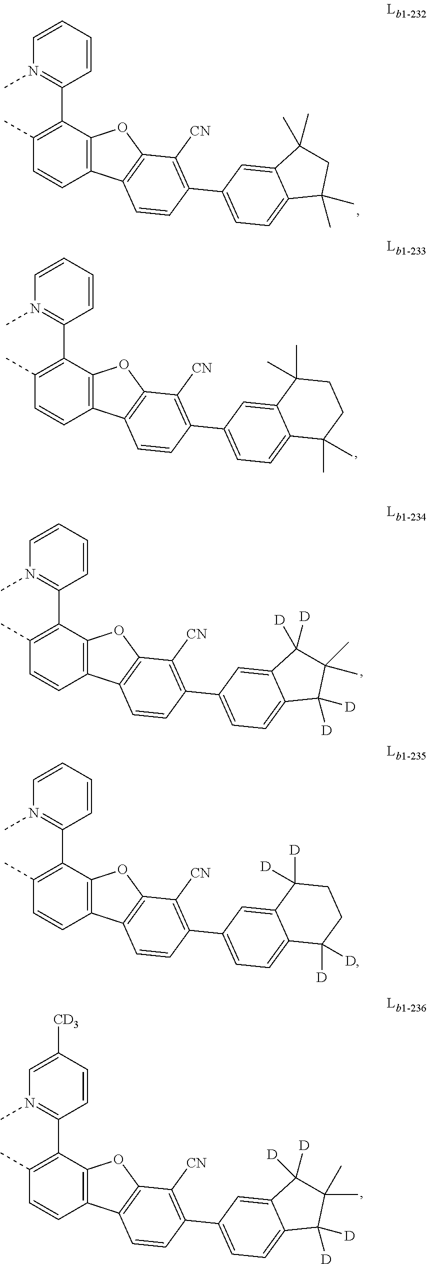 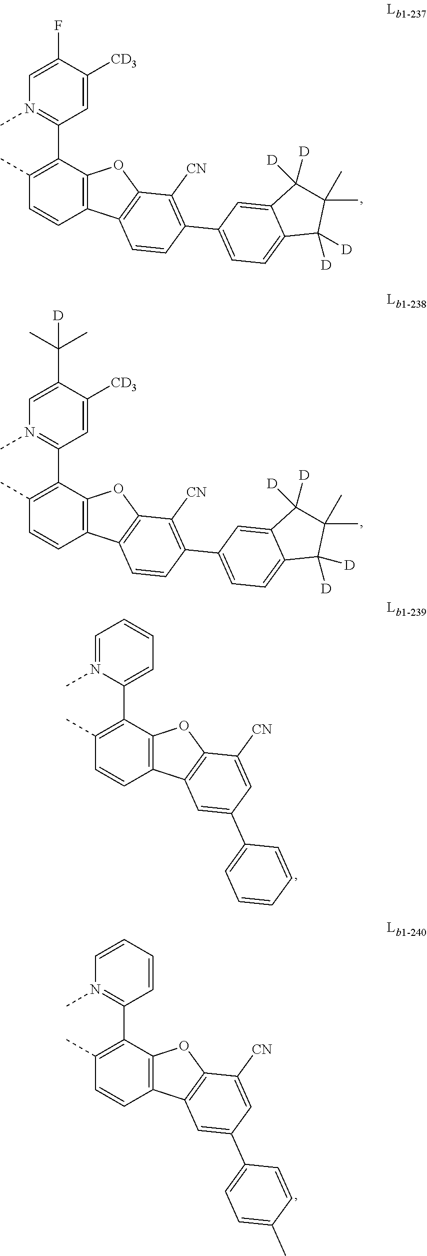 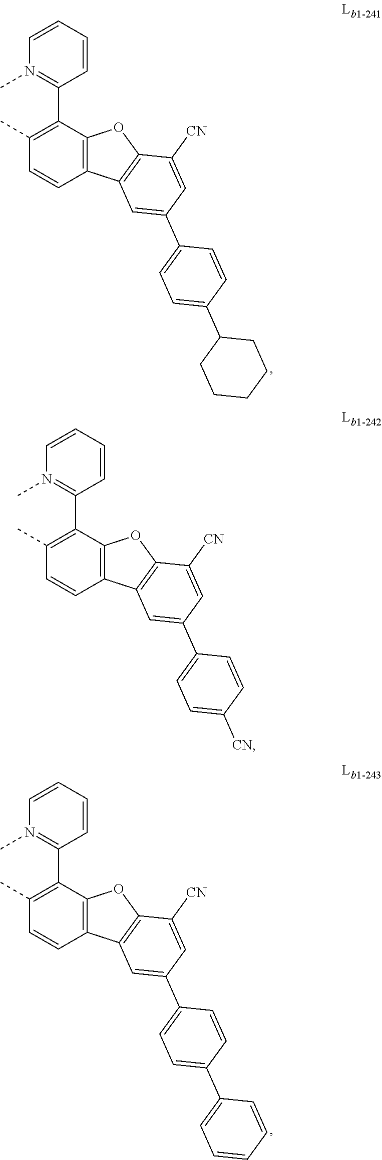 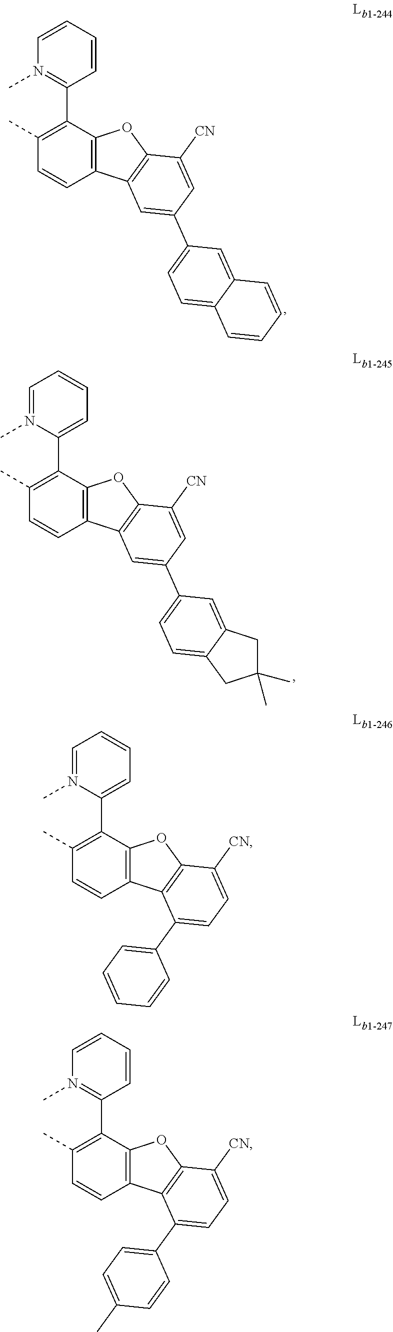 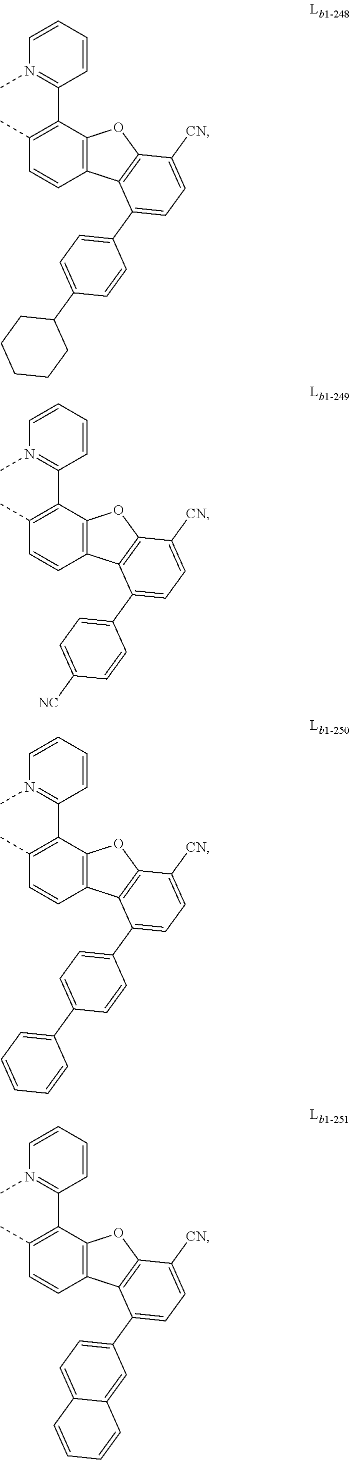 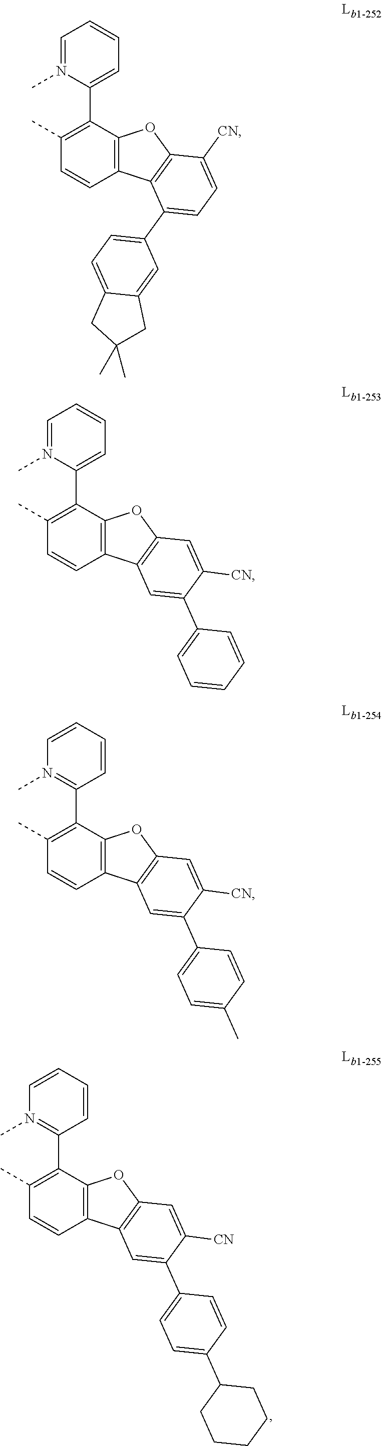 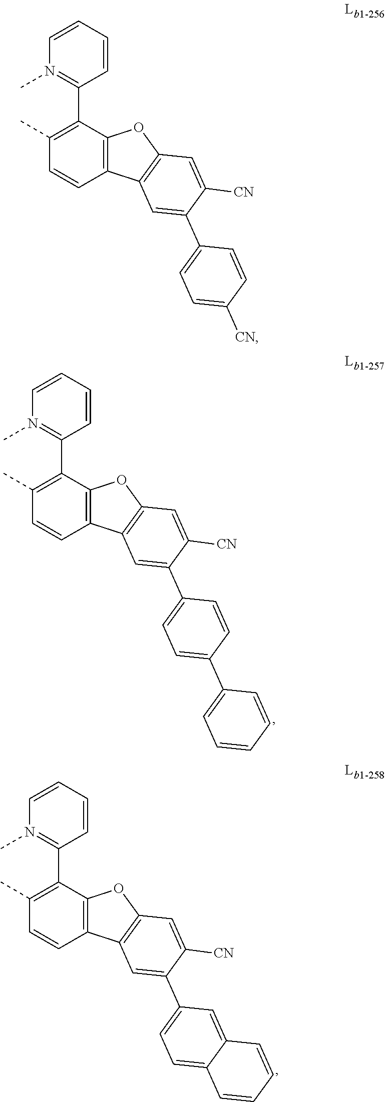 [chemical structure] [chemical structure] 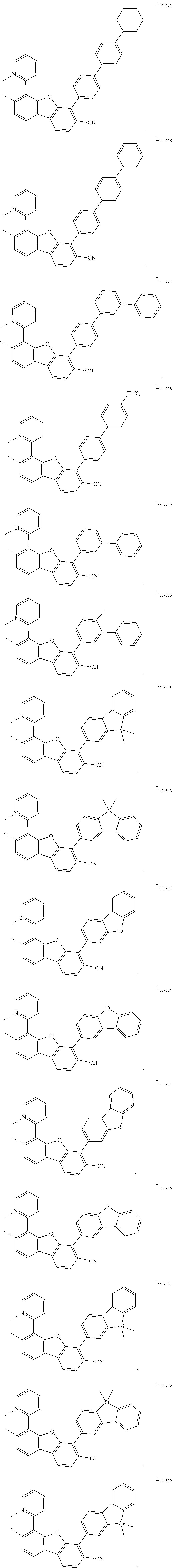 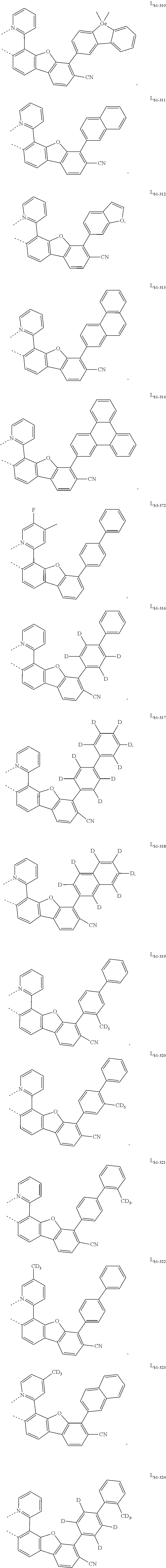 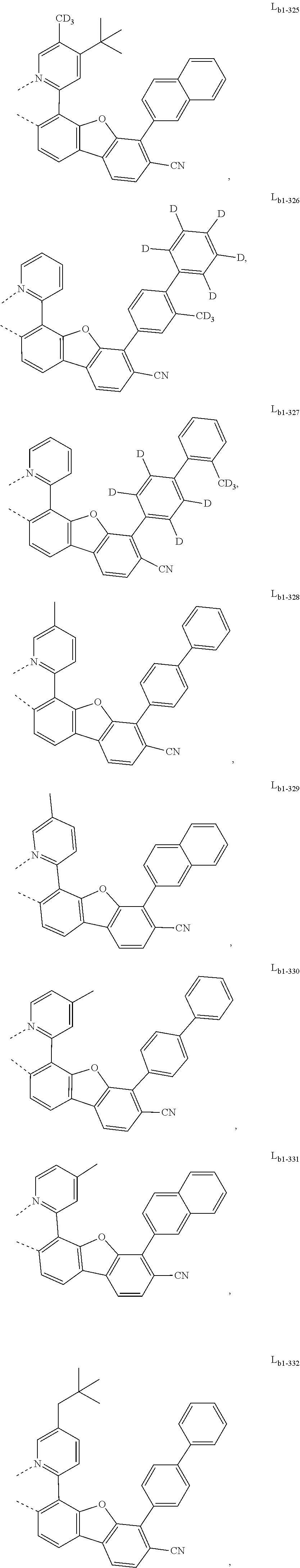 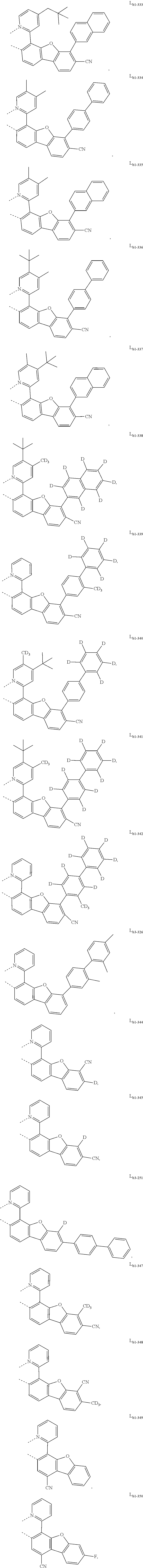 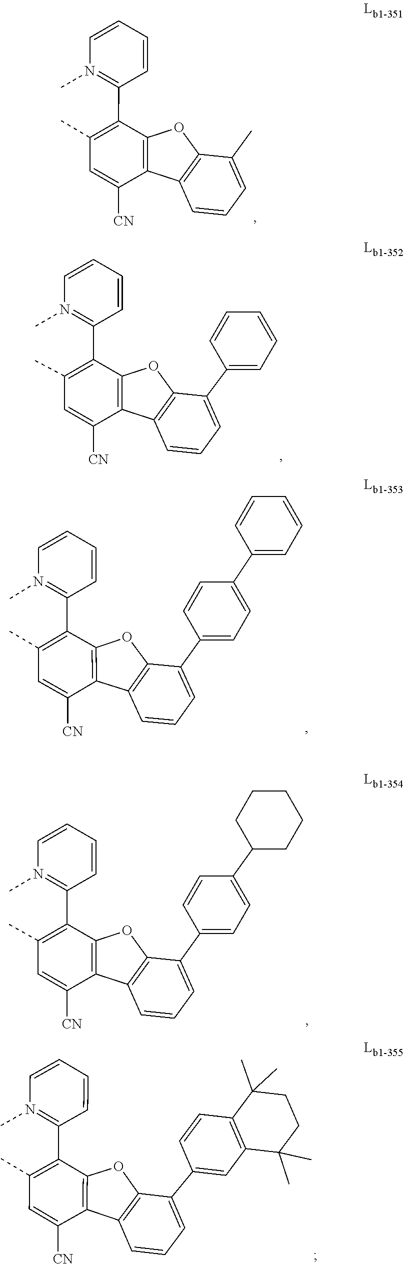 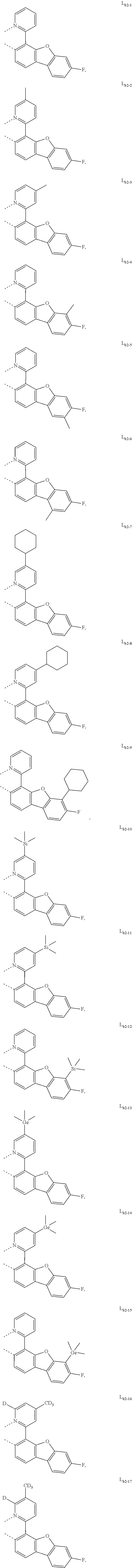 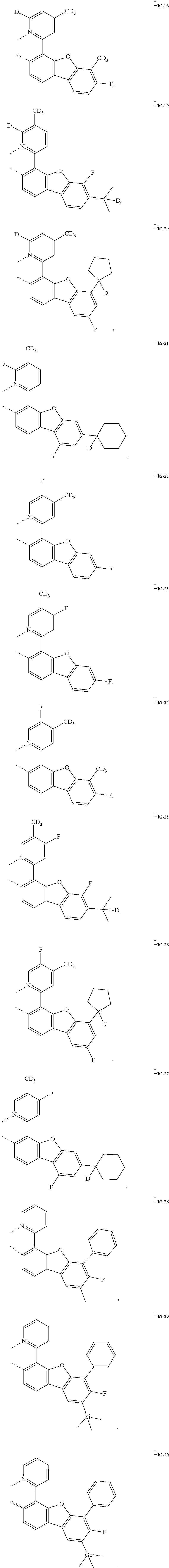 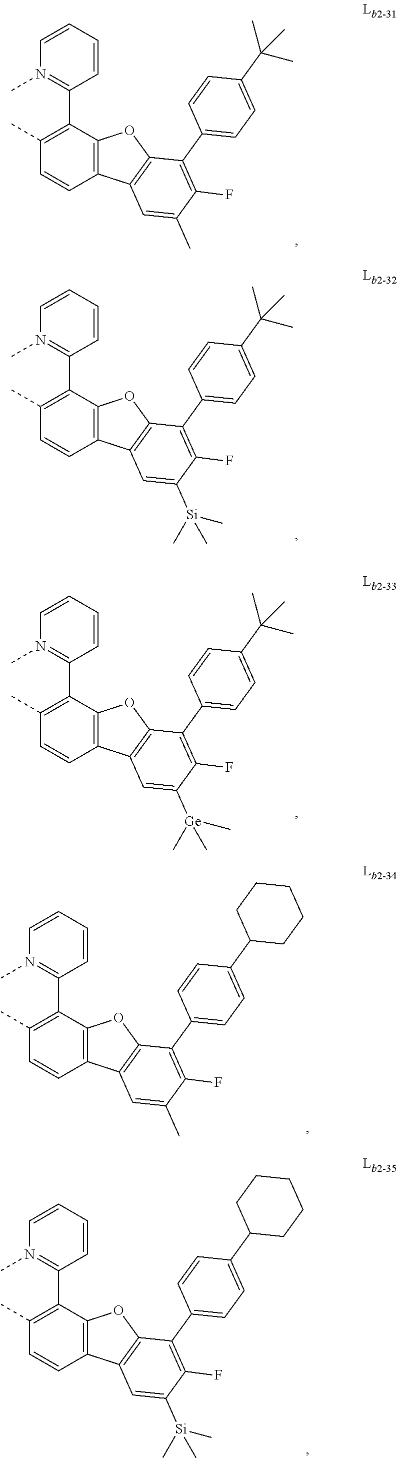 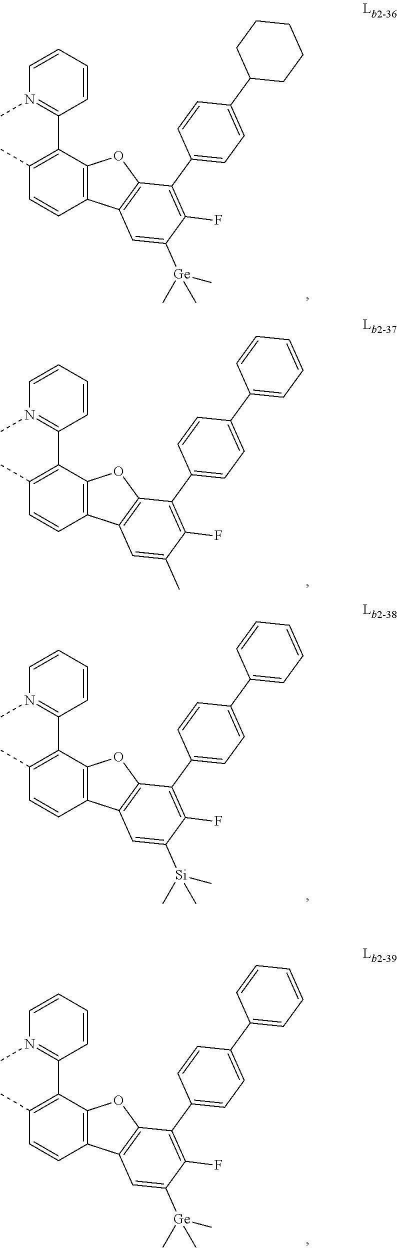 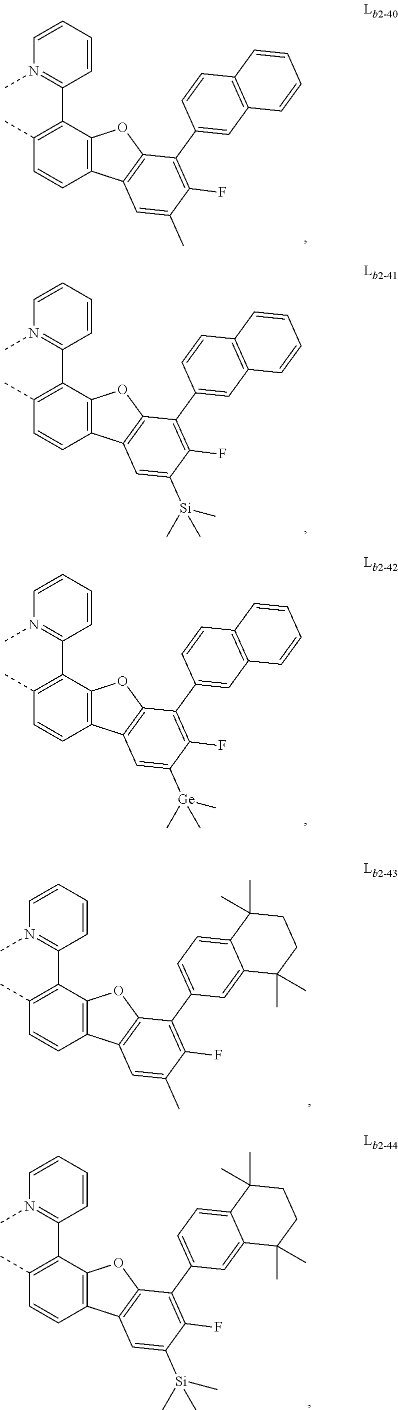 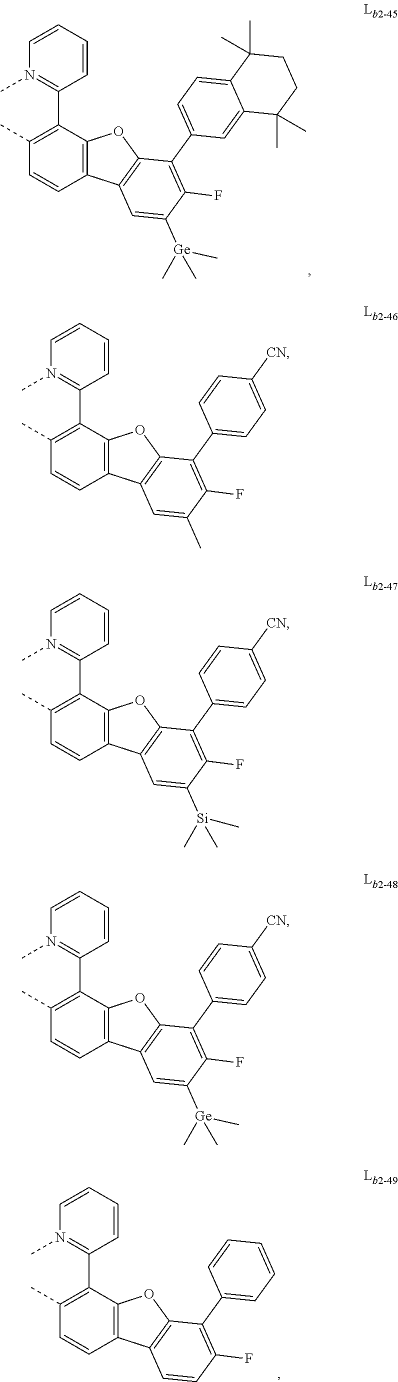 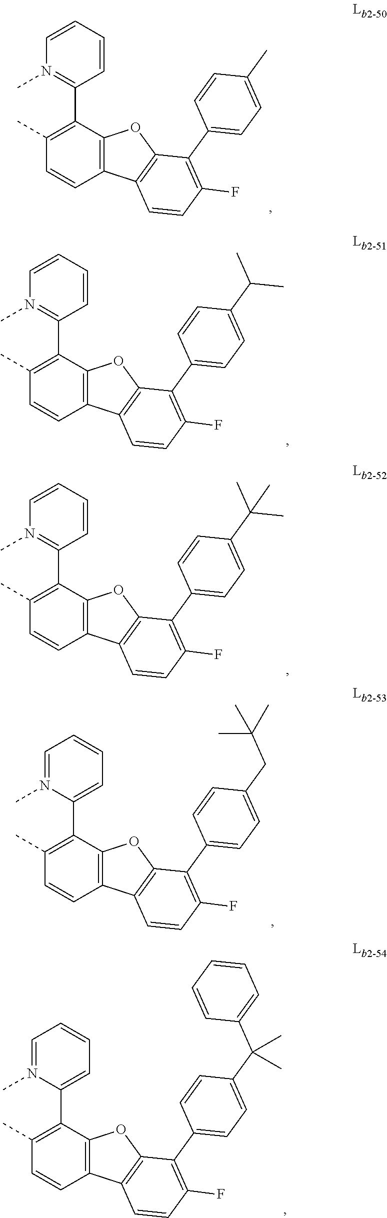 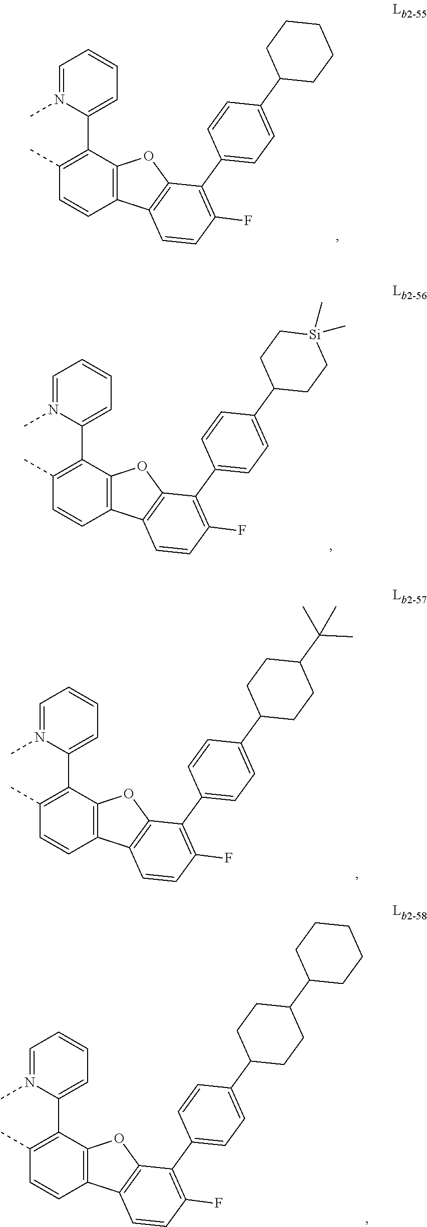 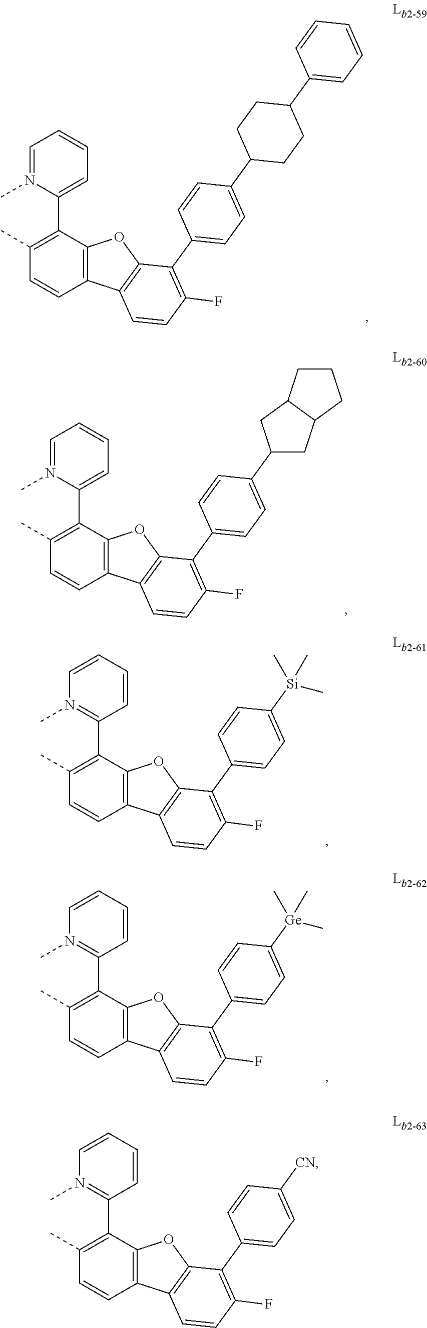 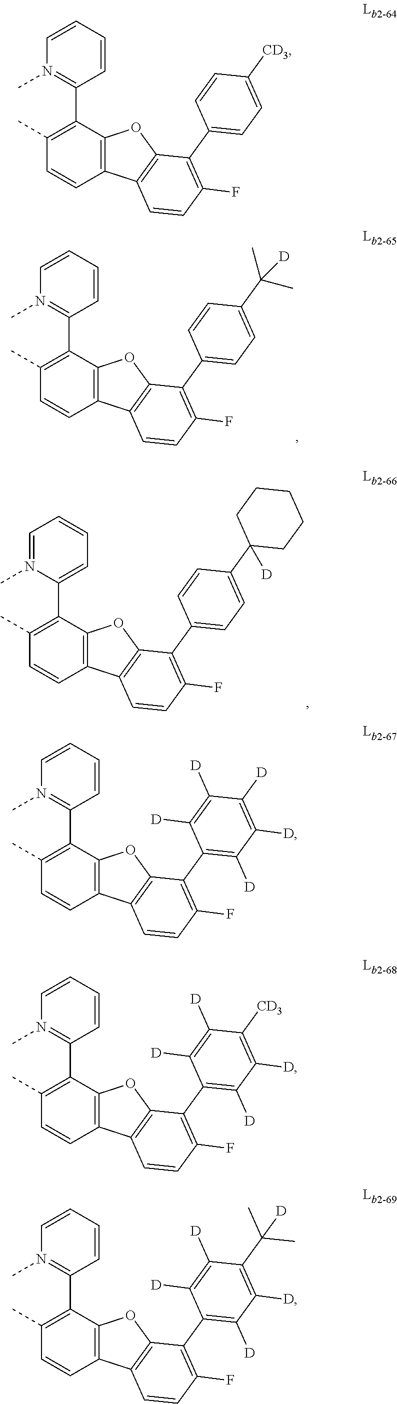 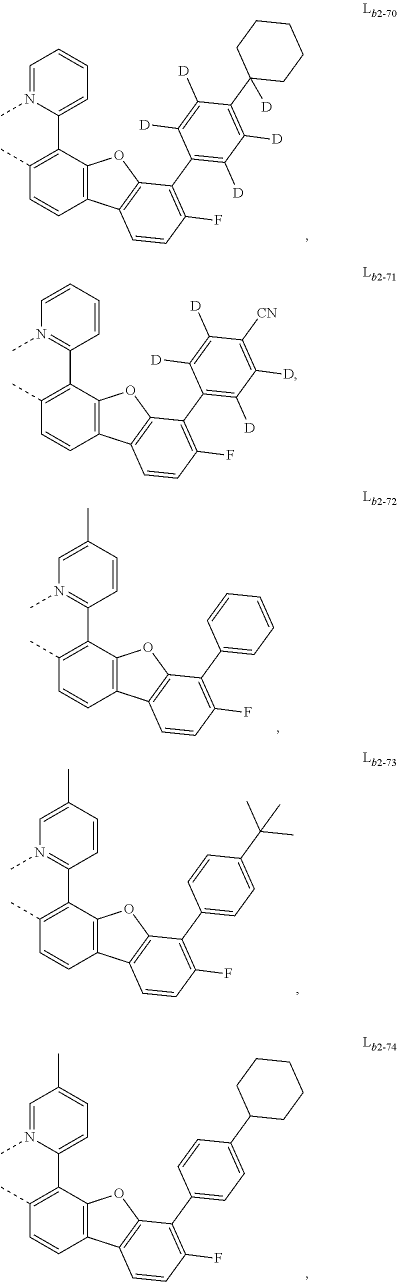 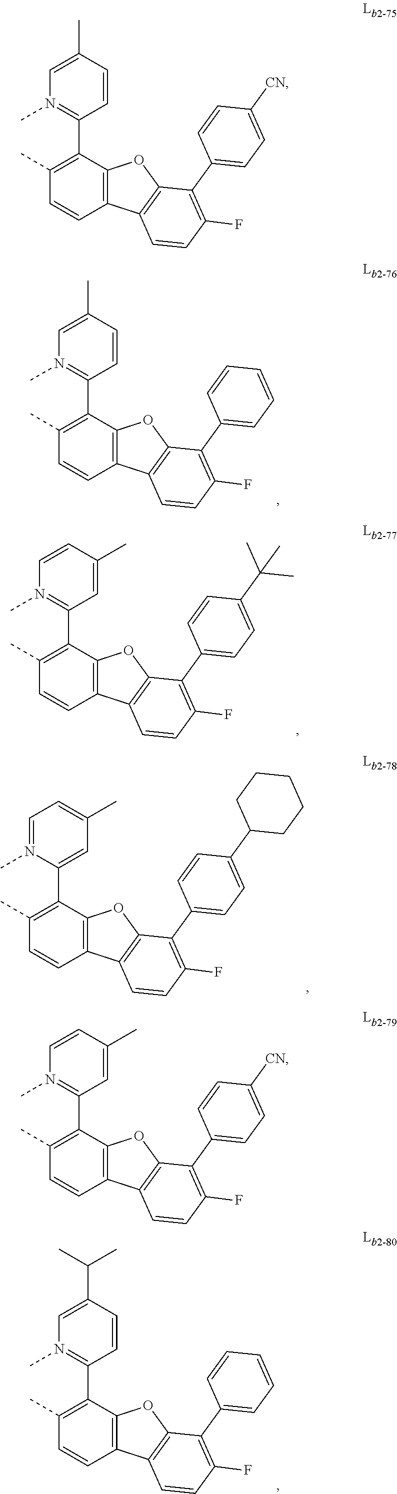 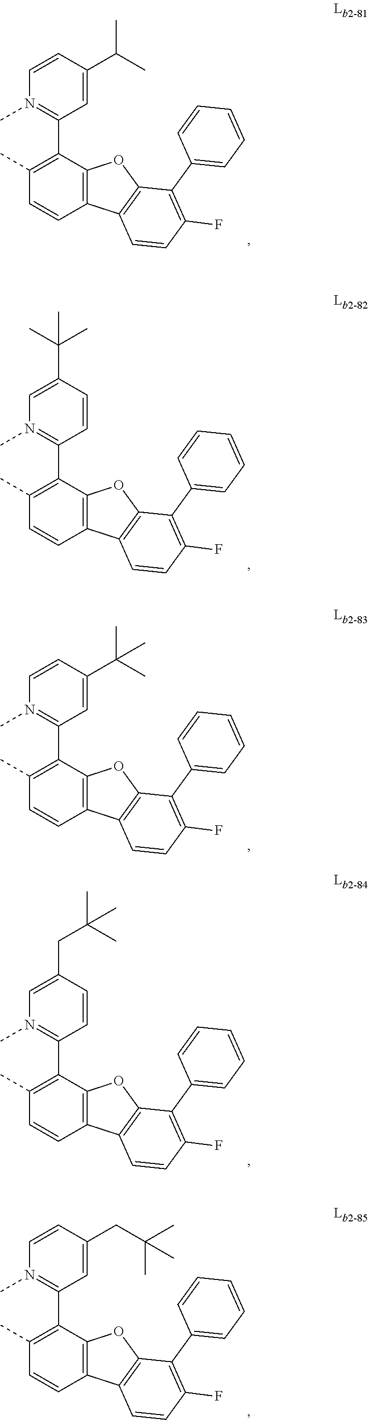 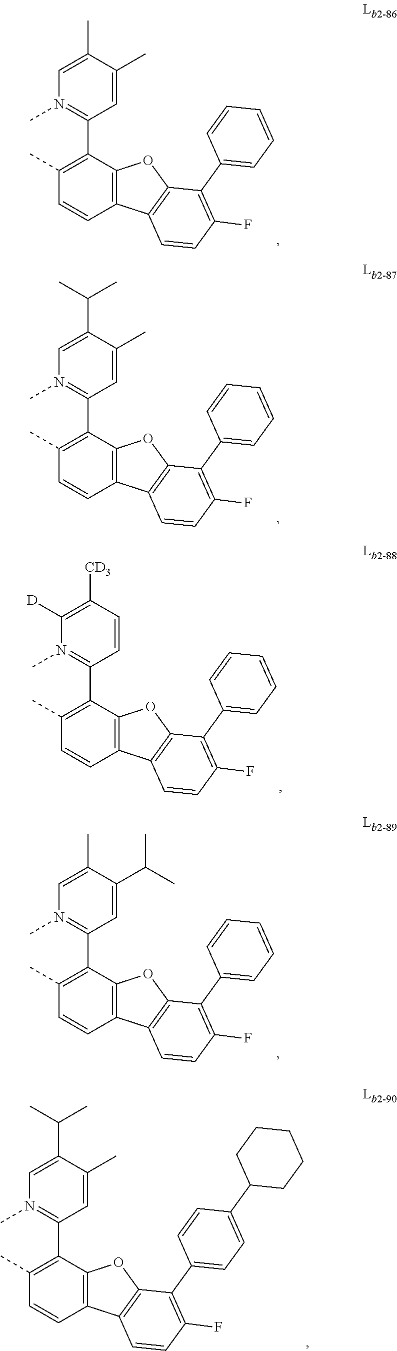 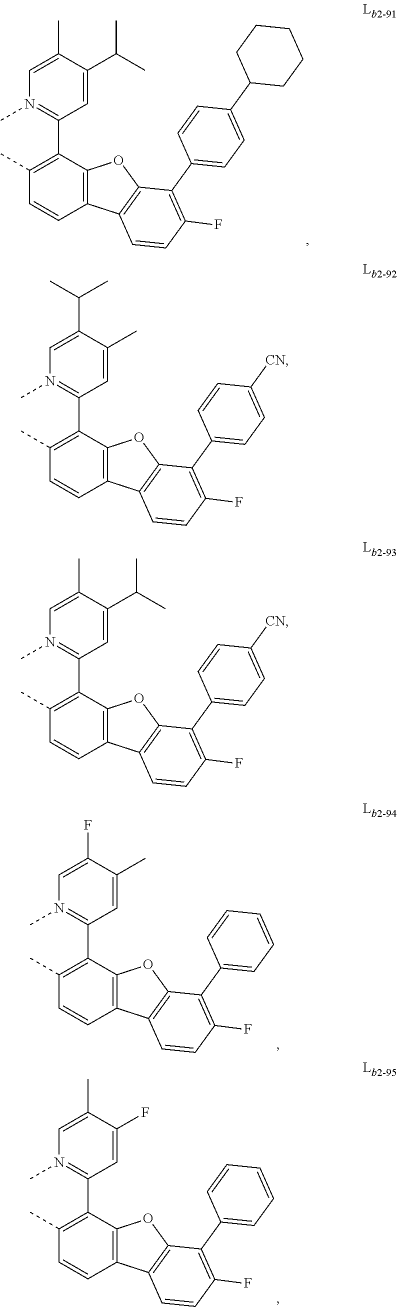 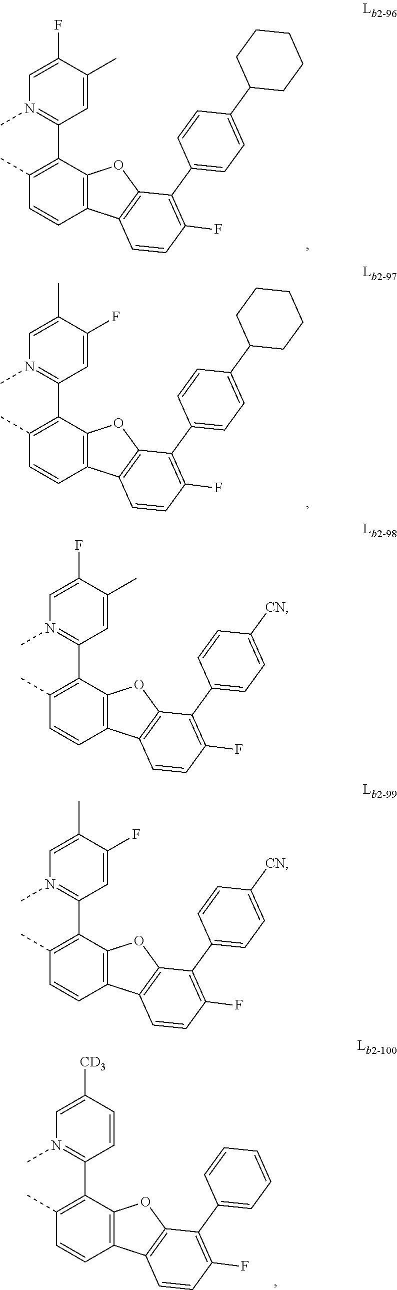 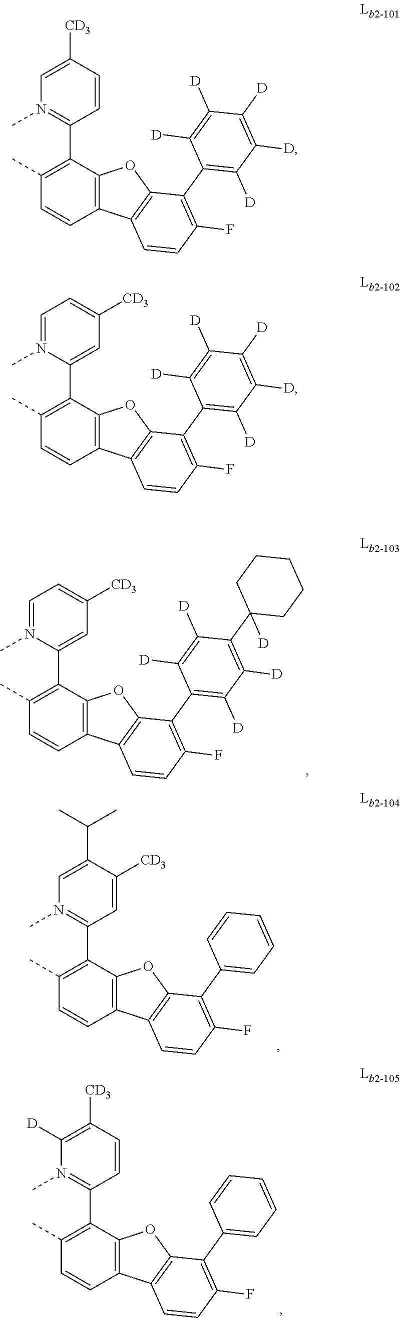 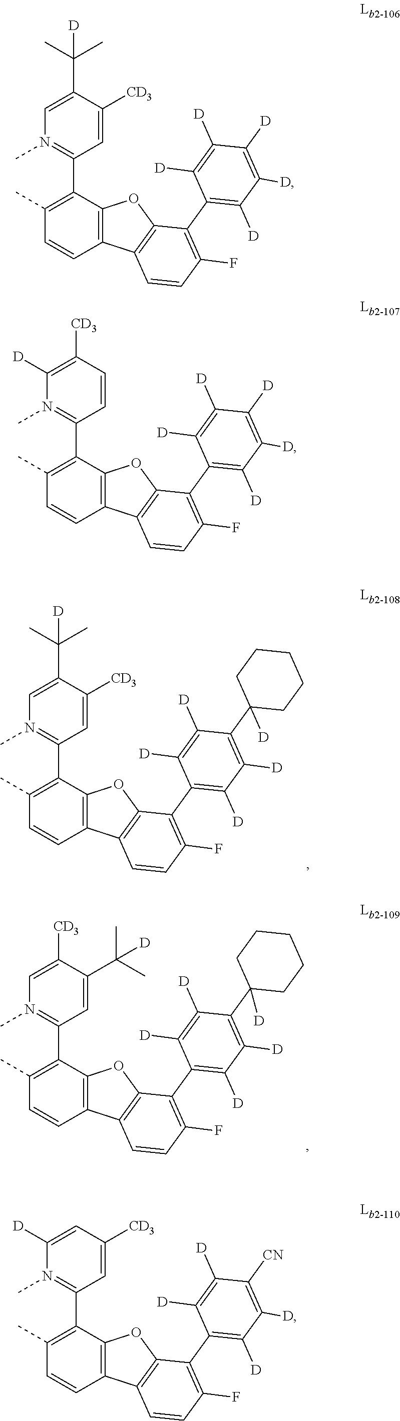 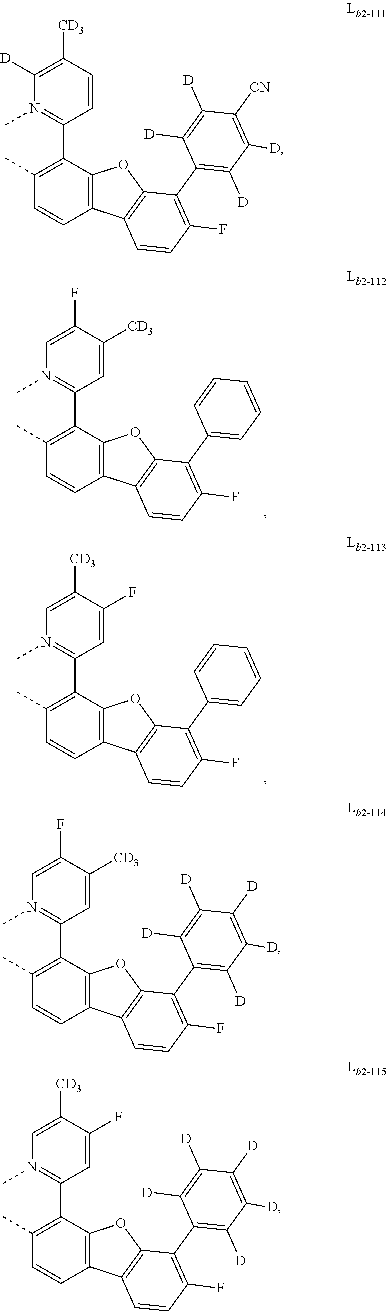 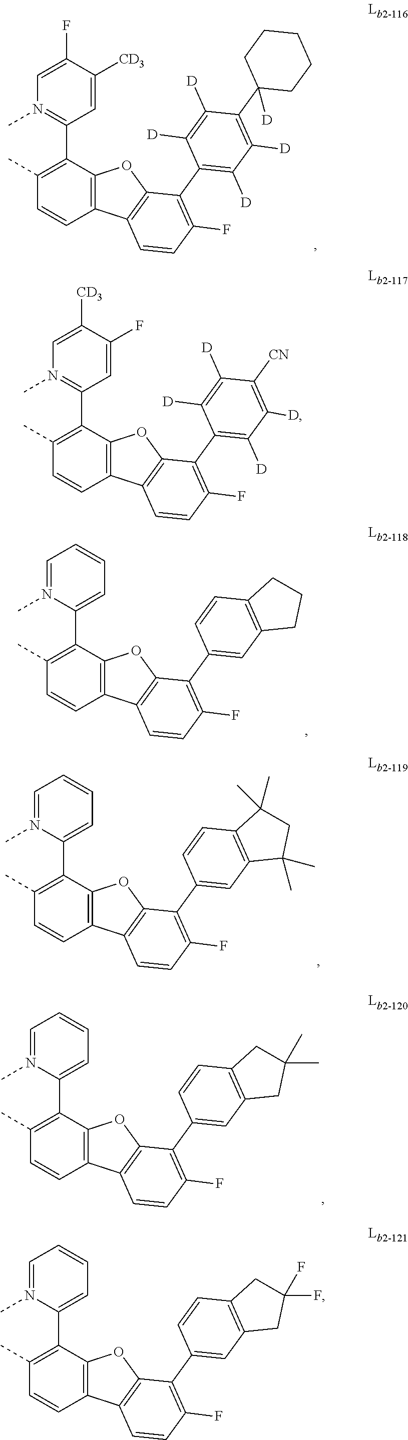 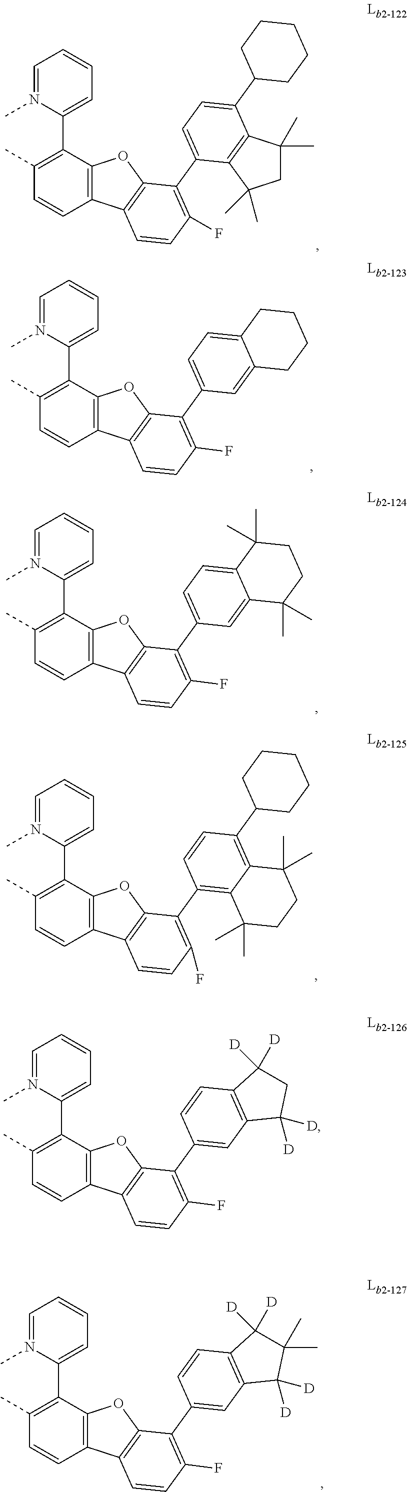 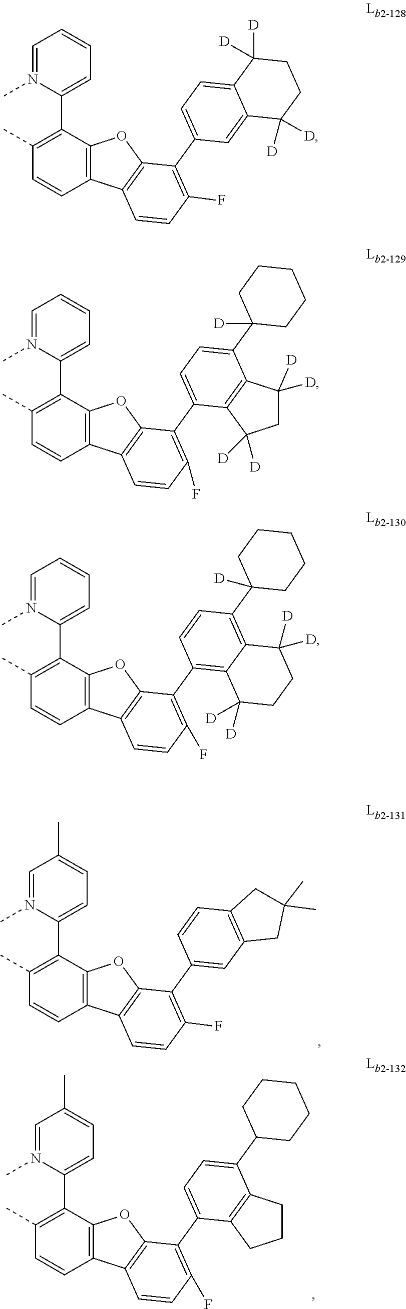 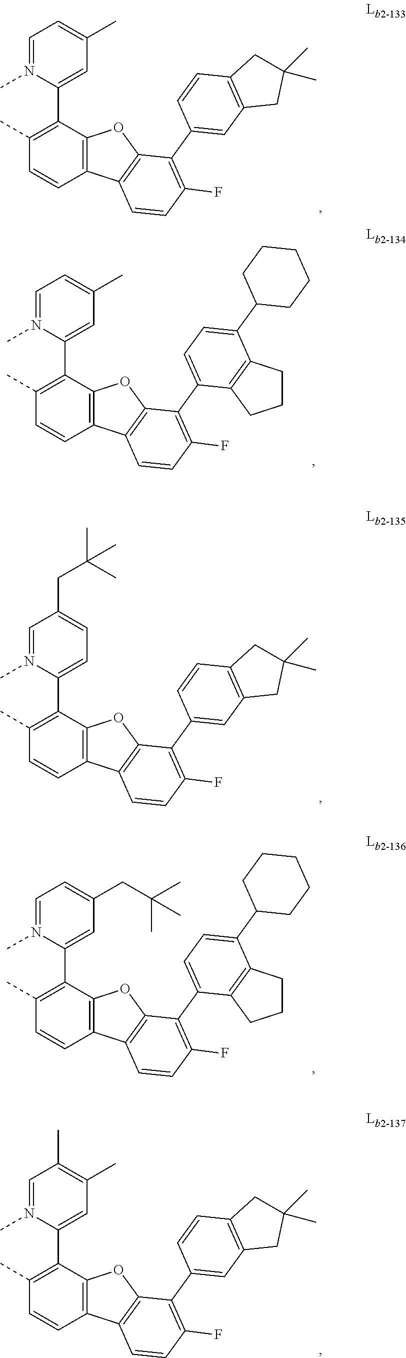 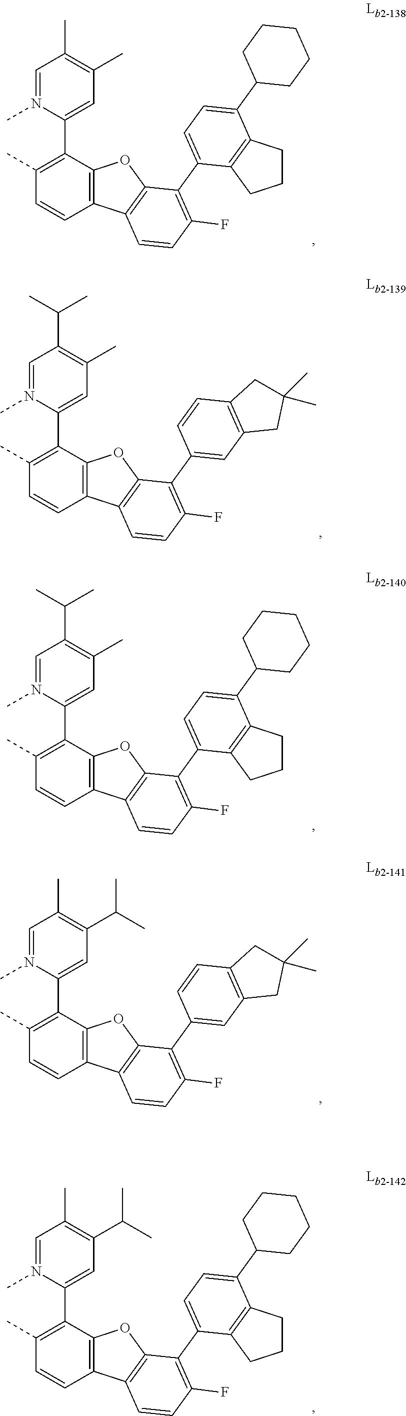 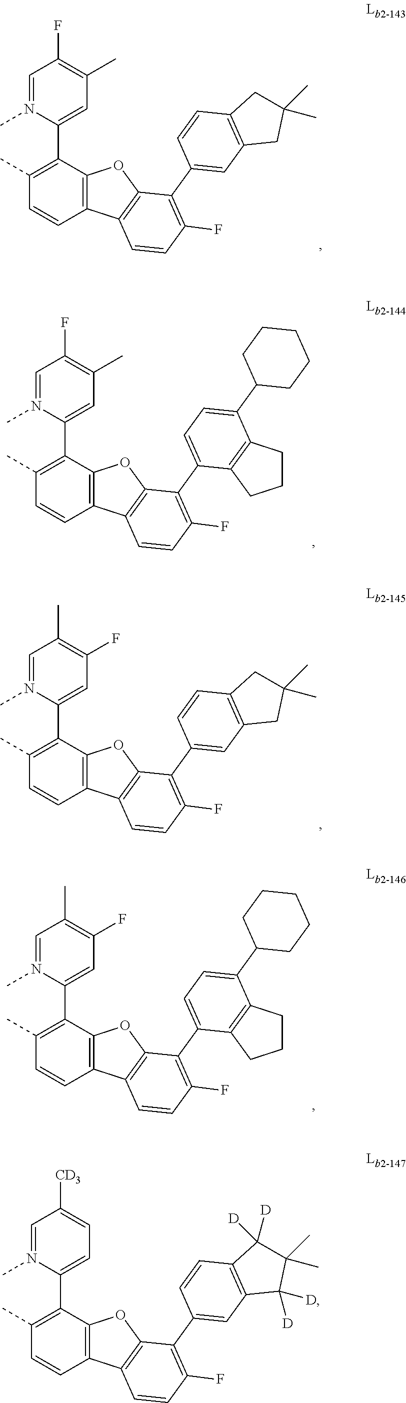 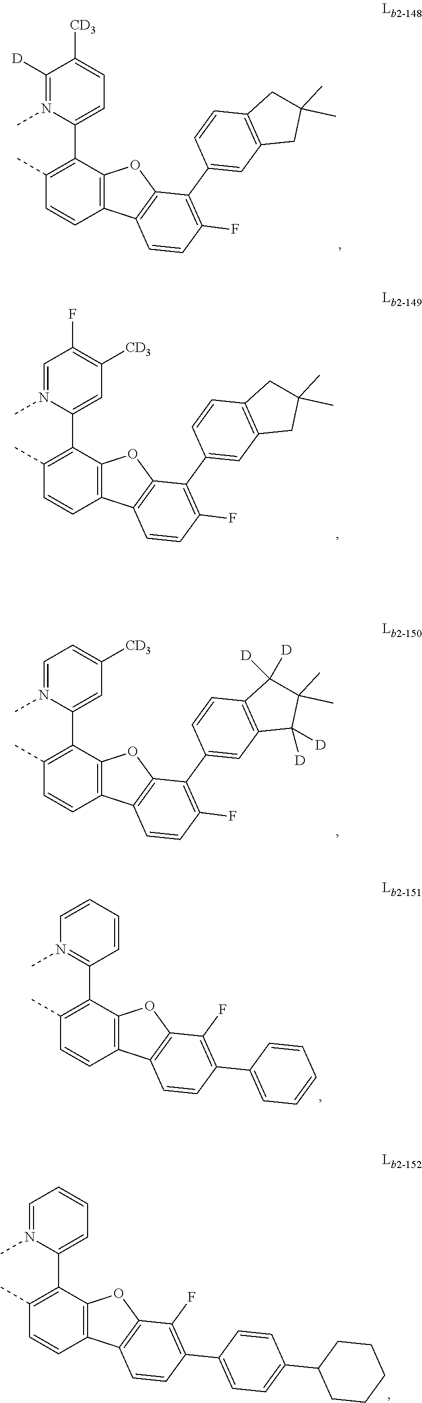 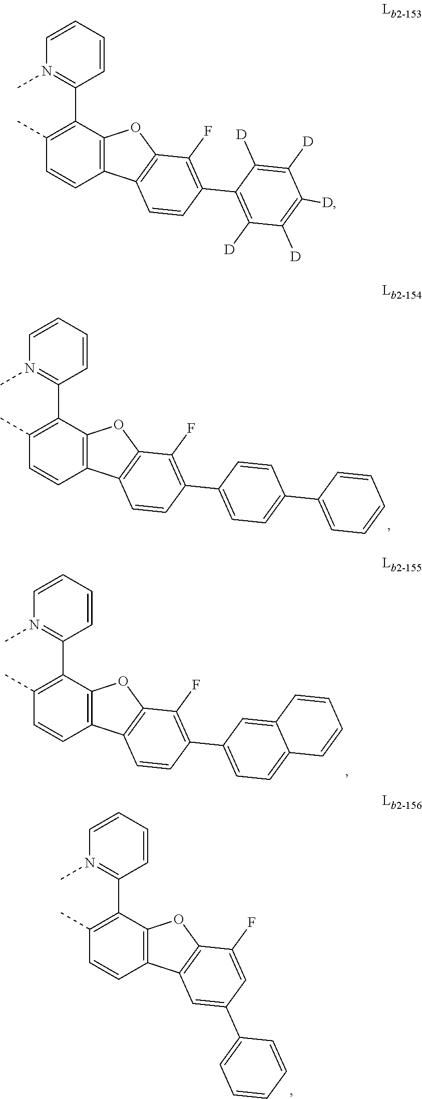 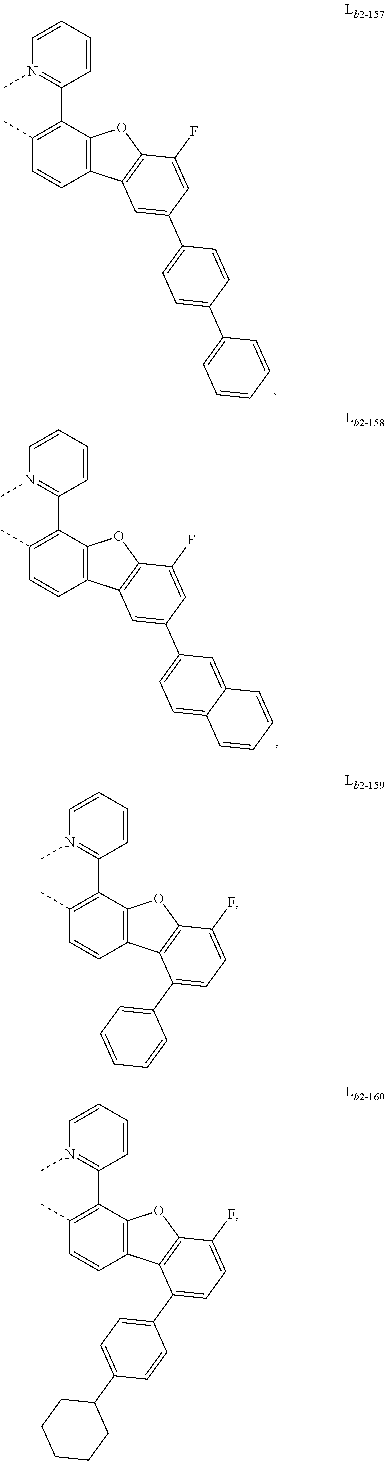 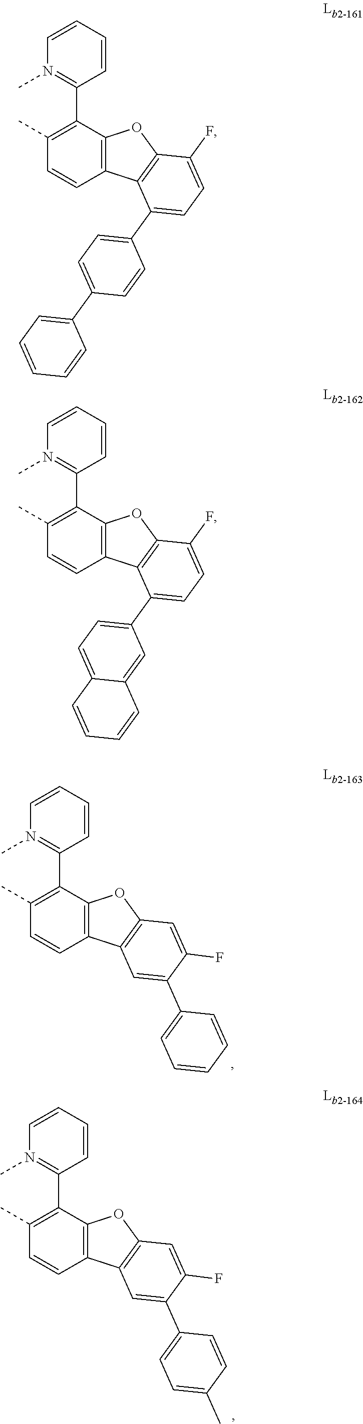 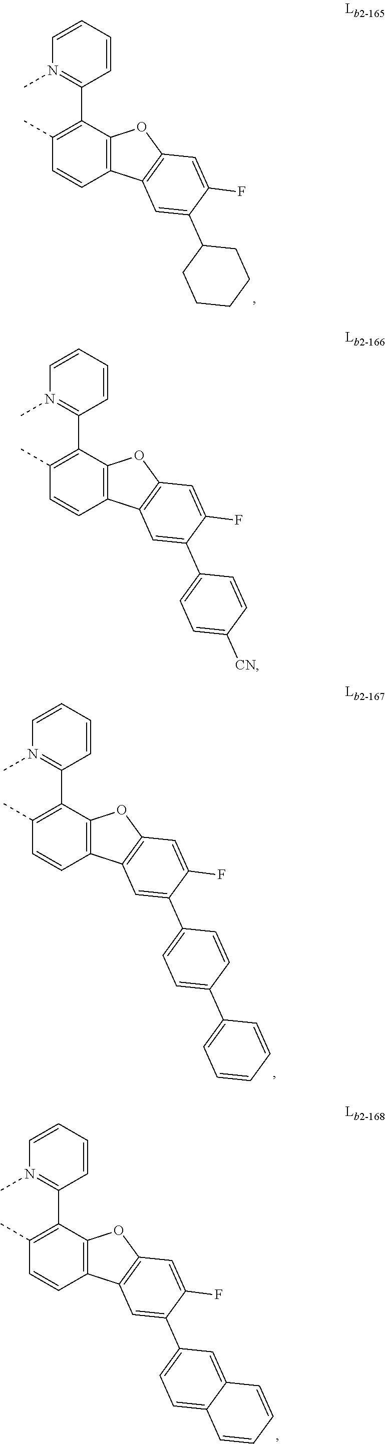 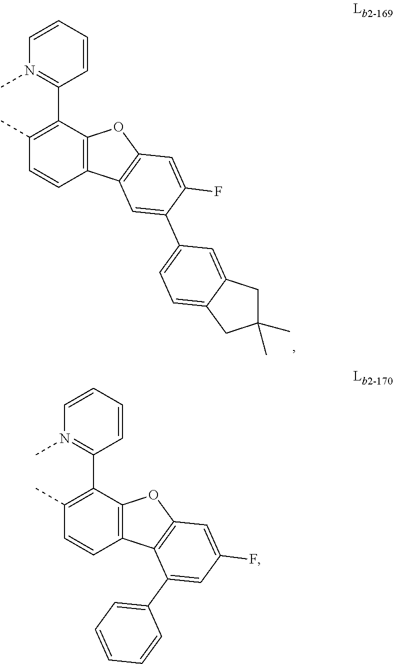 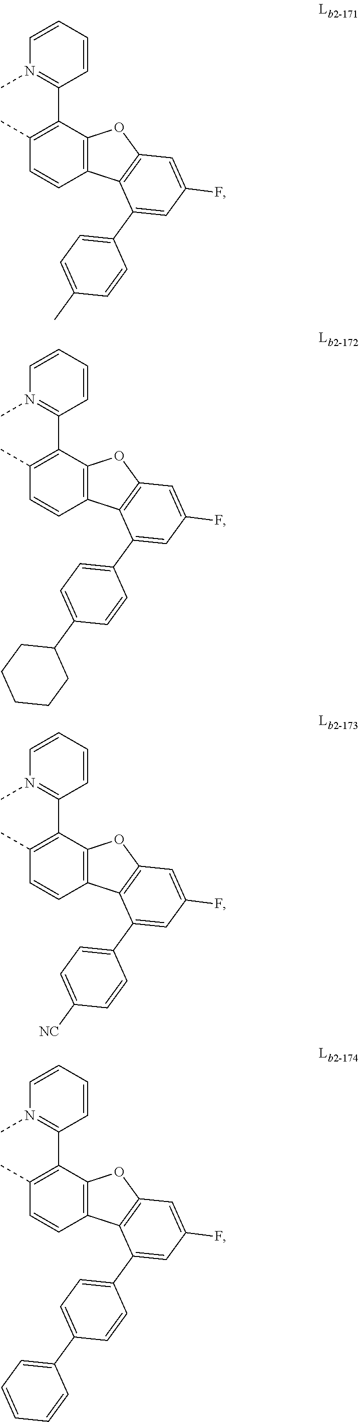 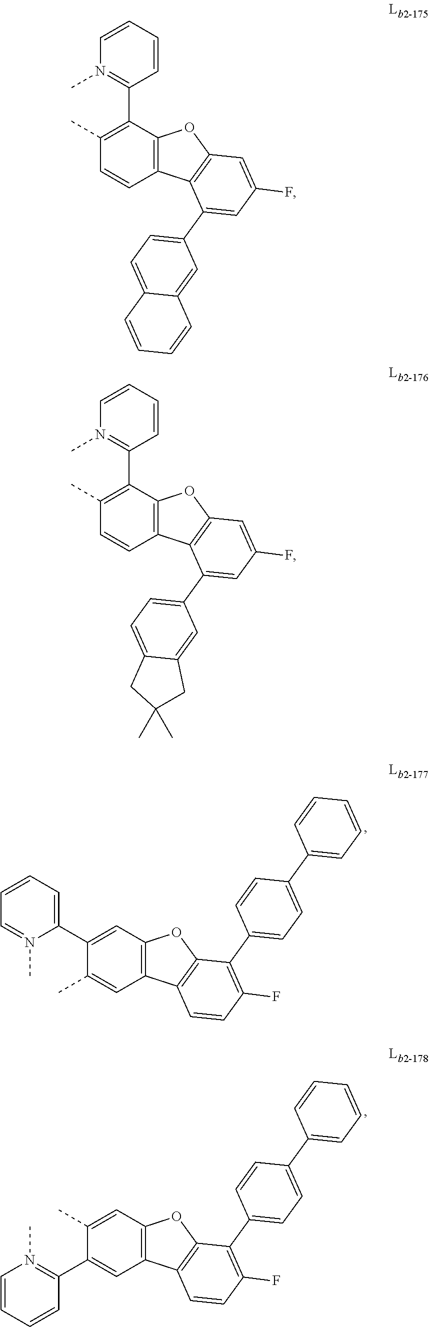 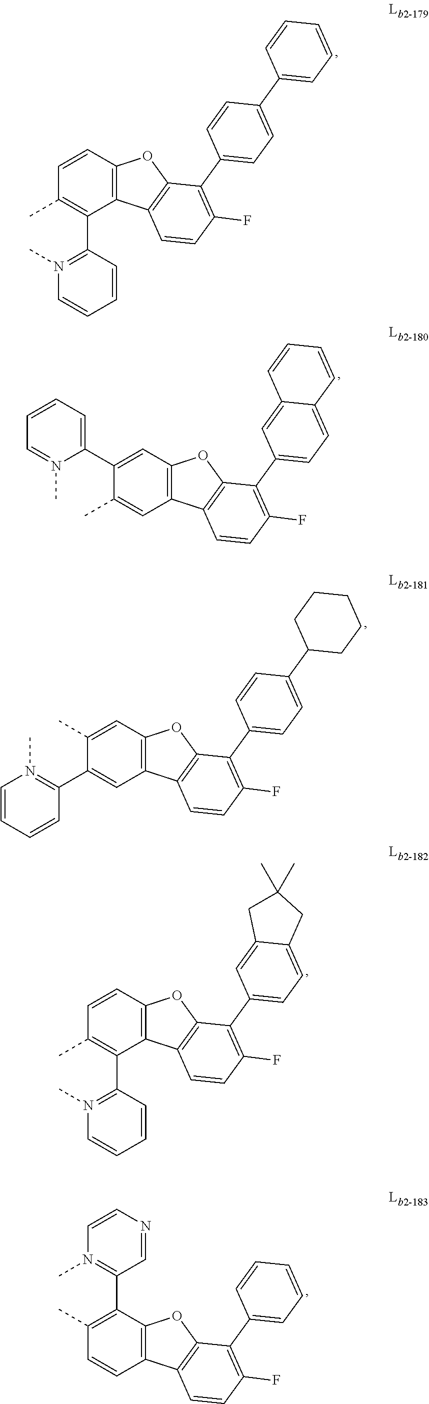 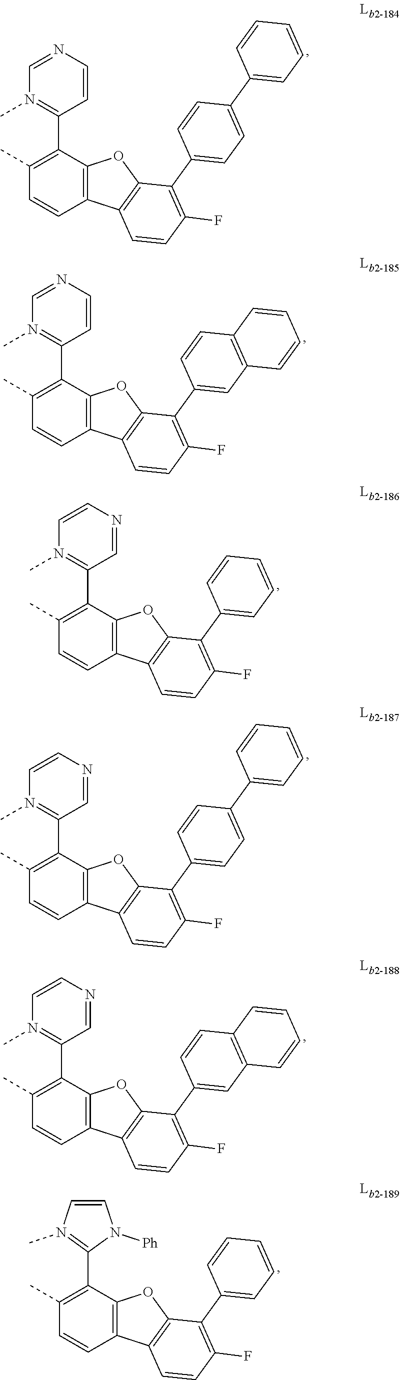 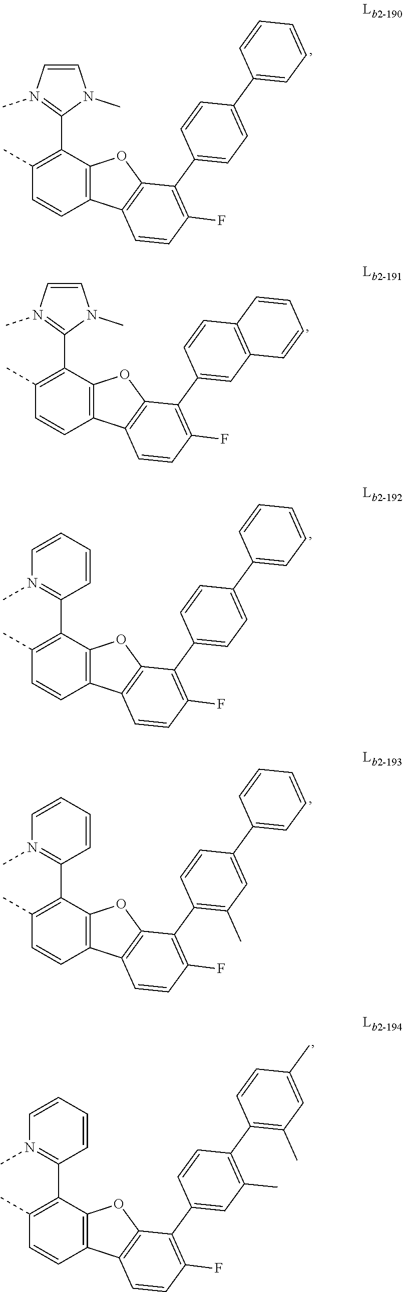 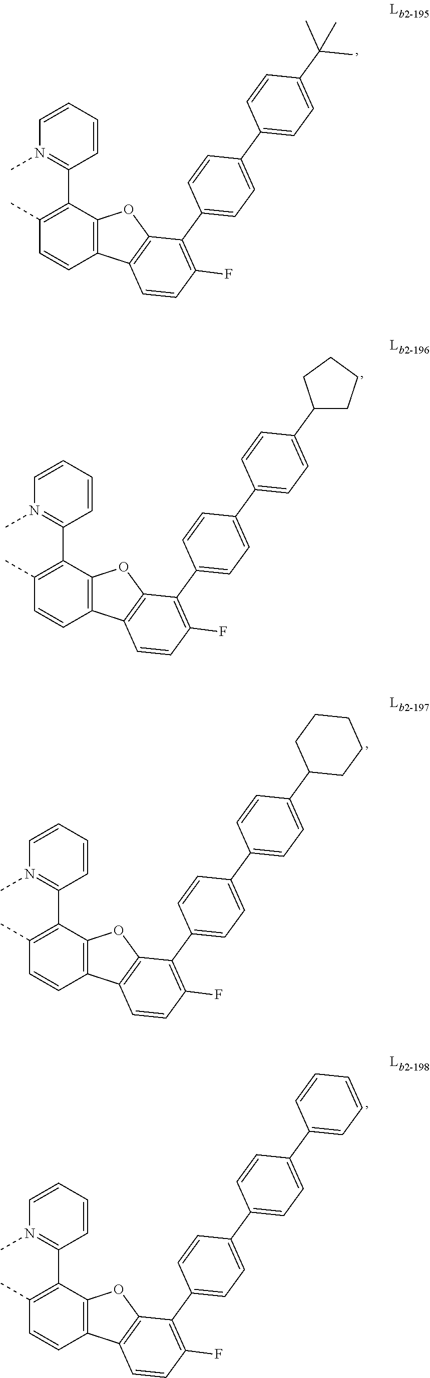 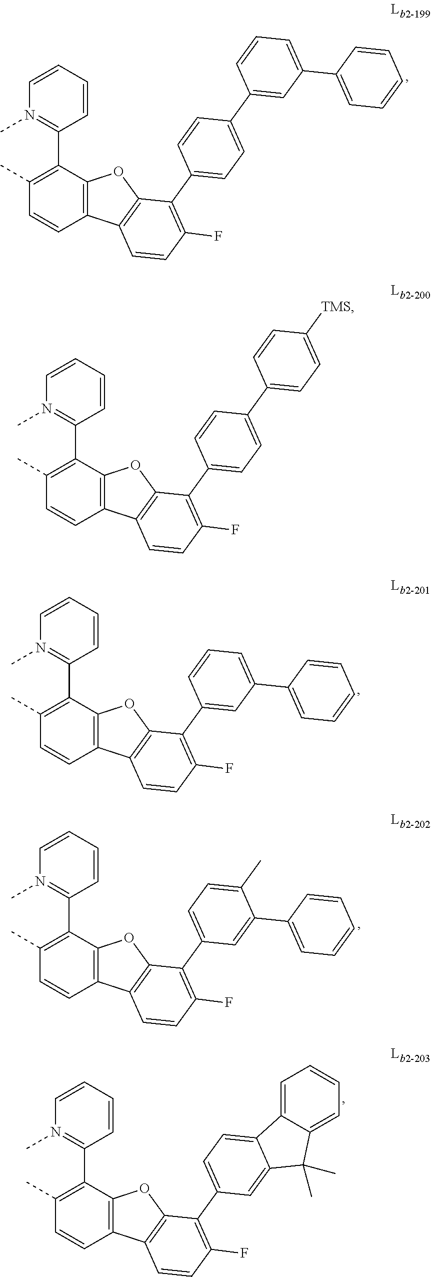 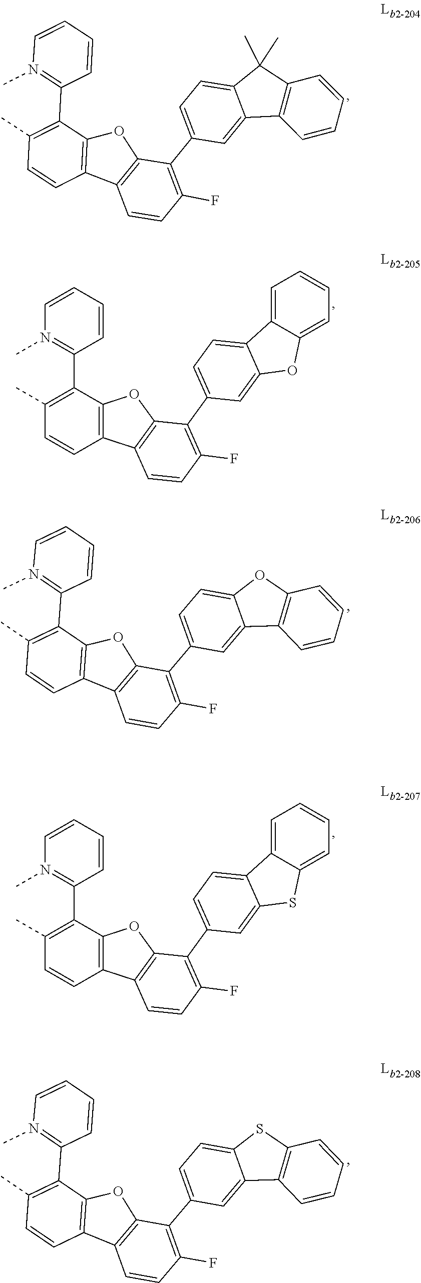 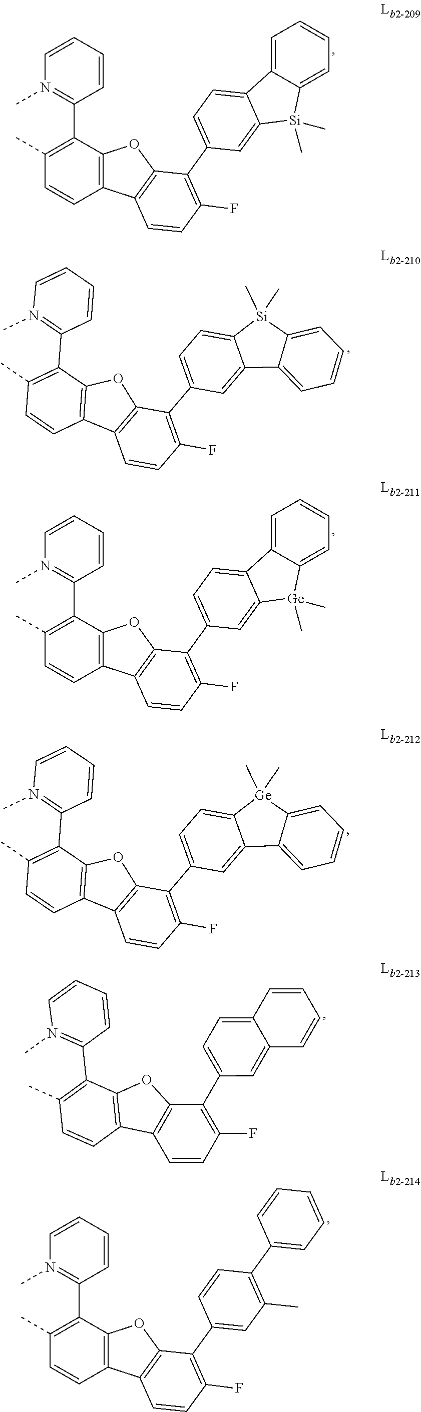 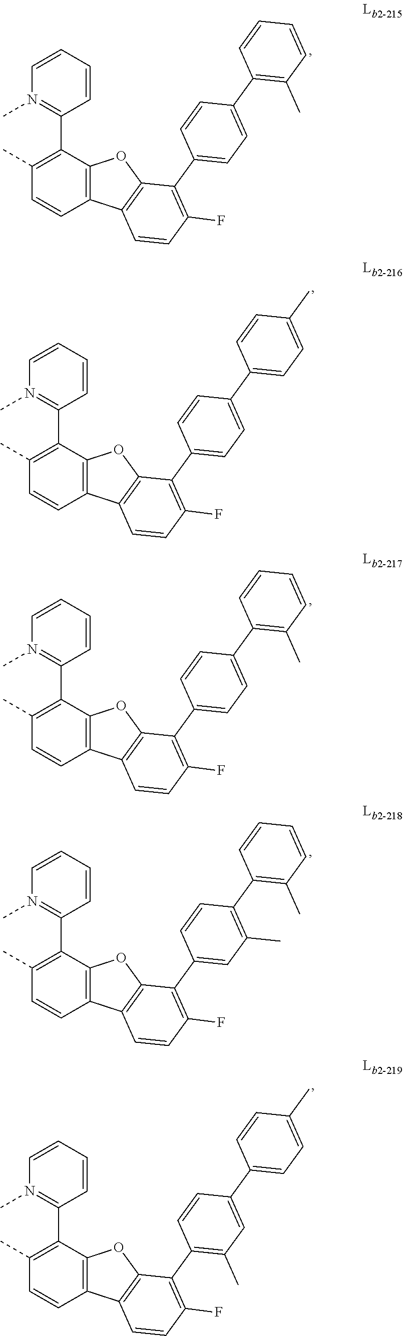 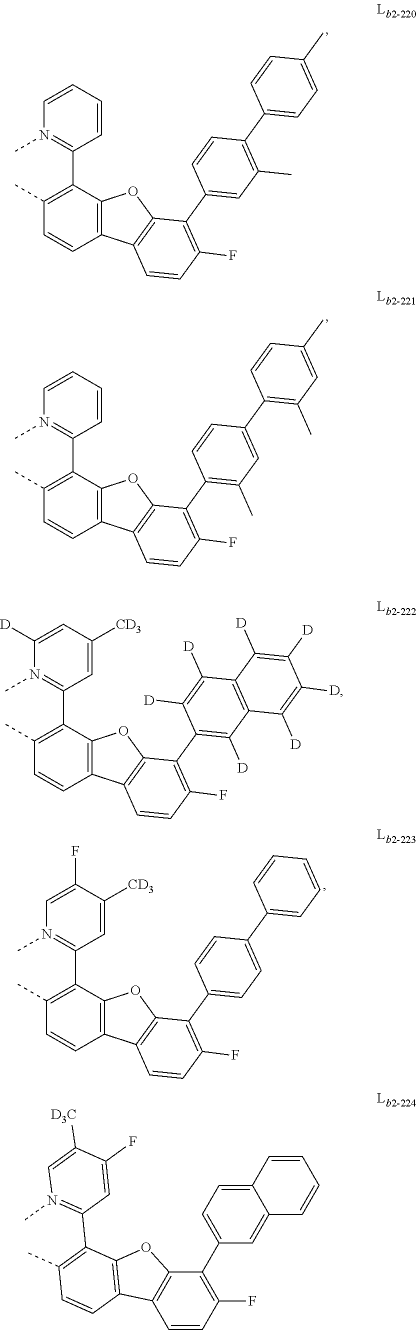 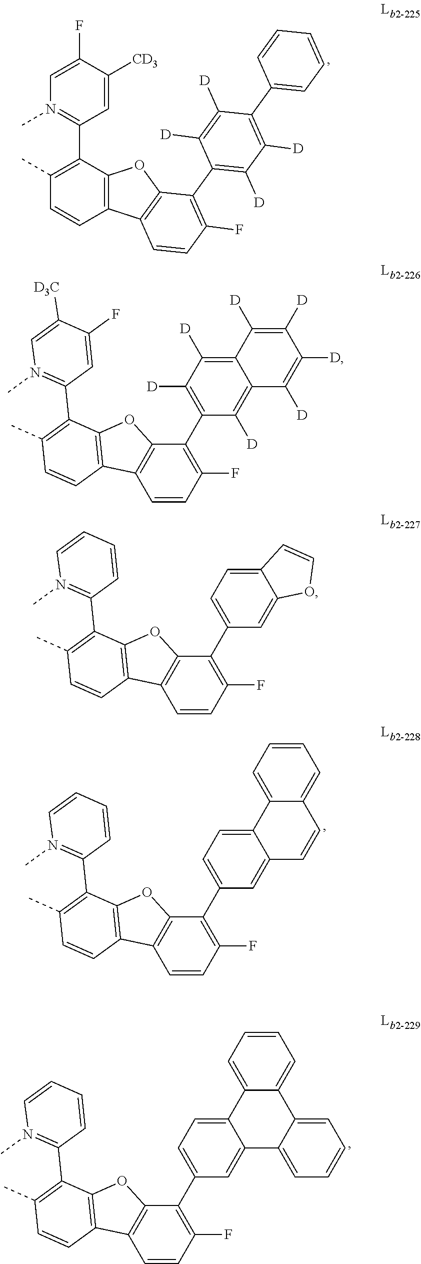 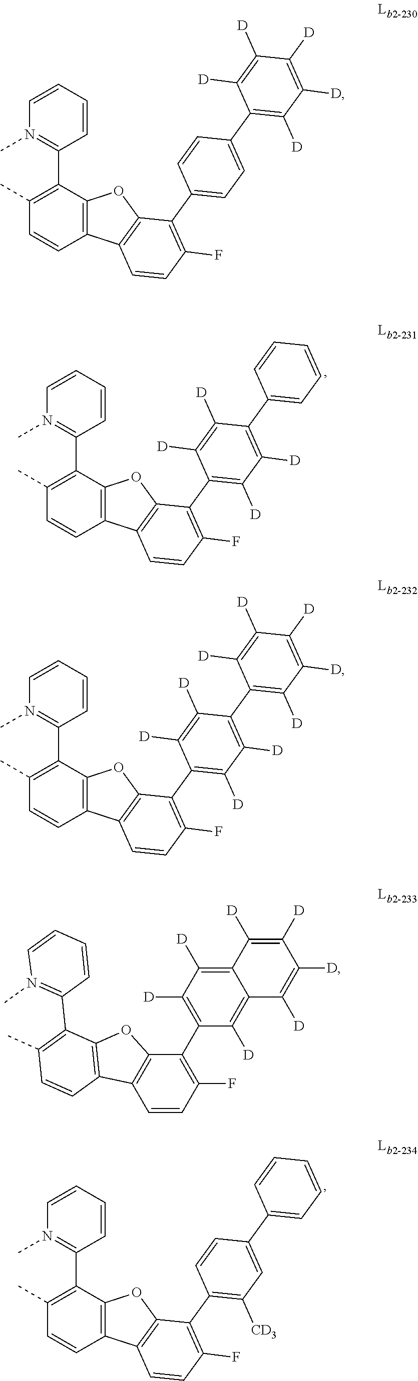 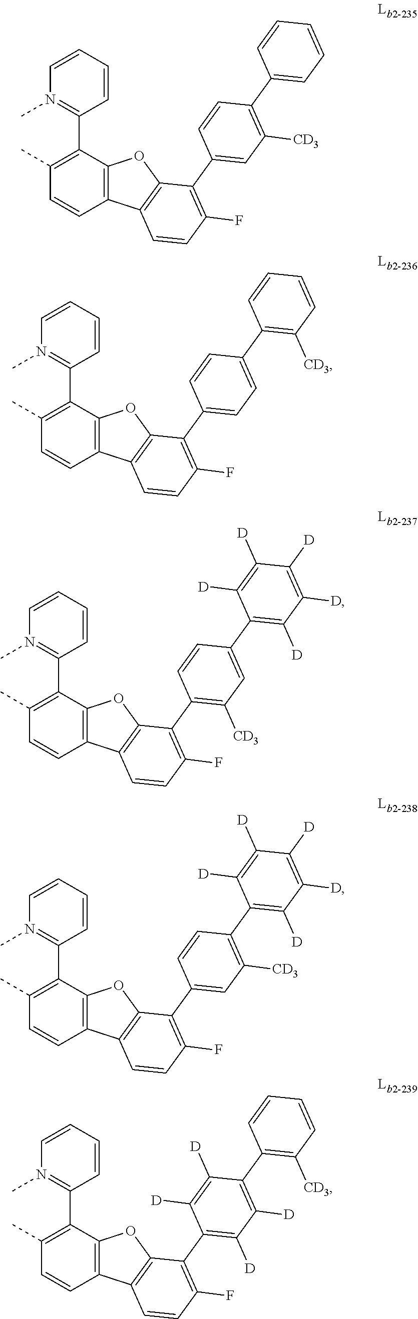 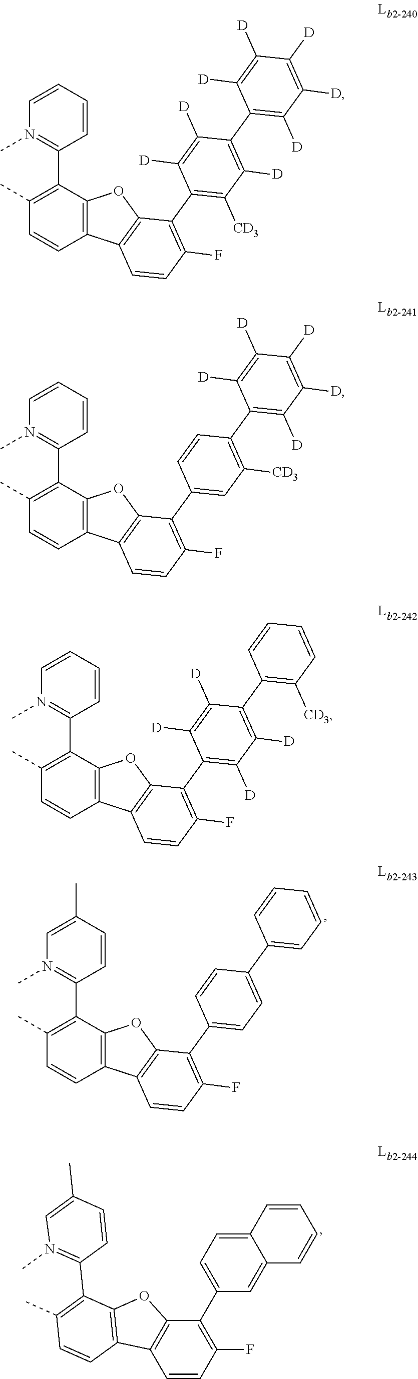 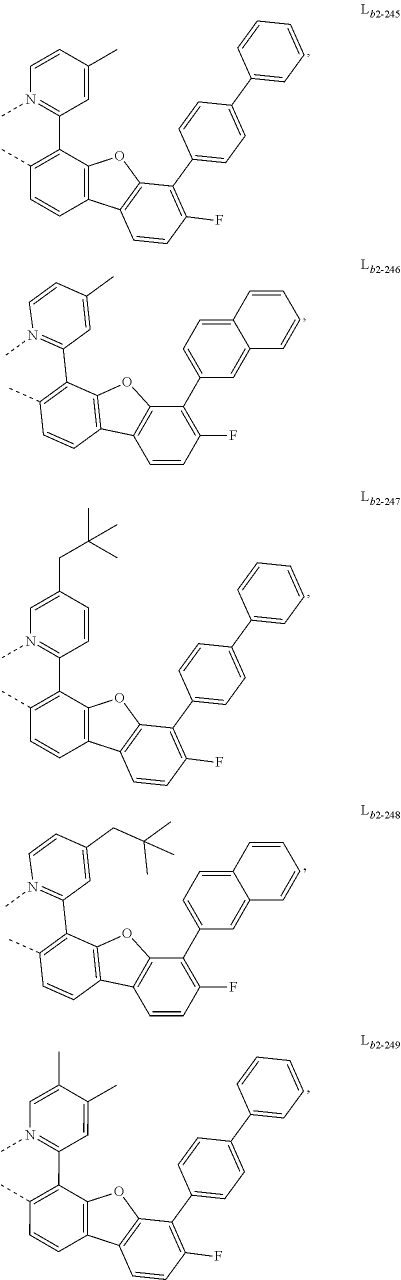 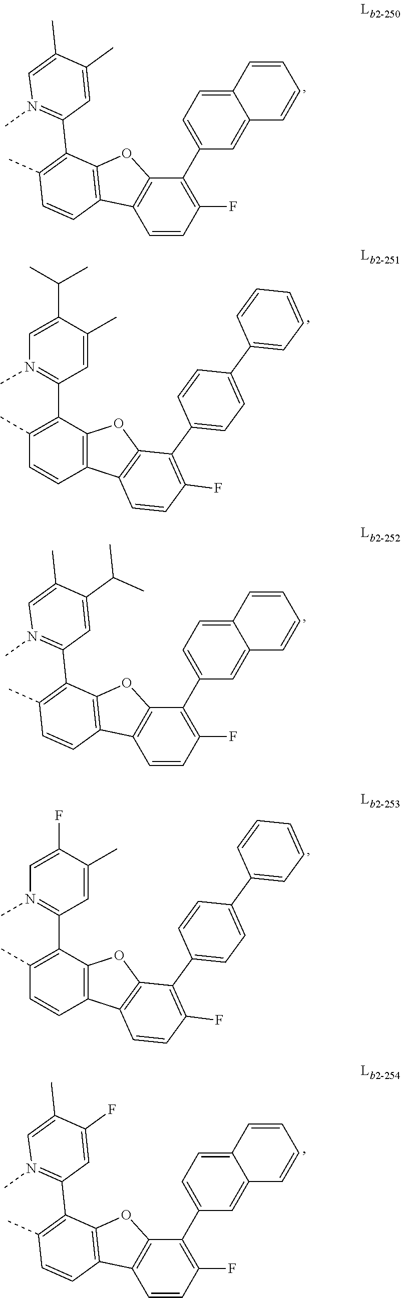 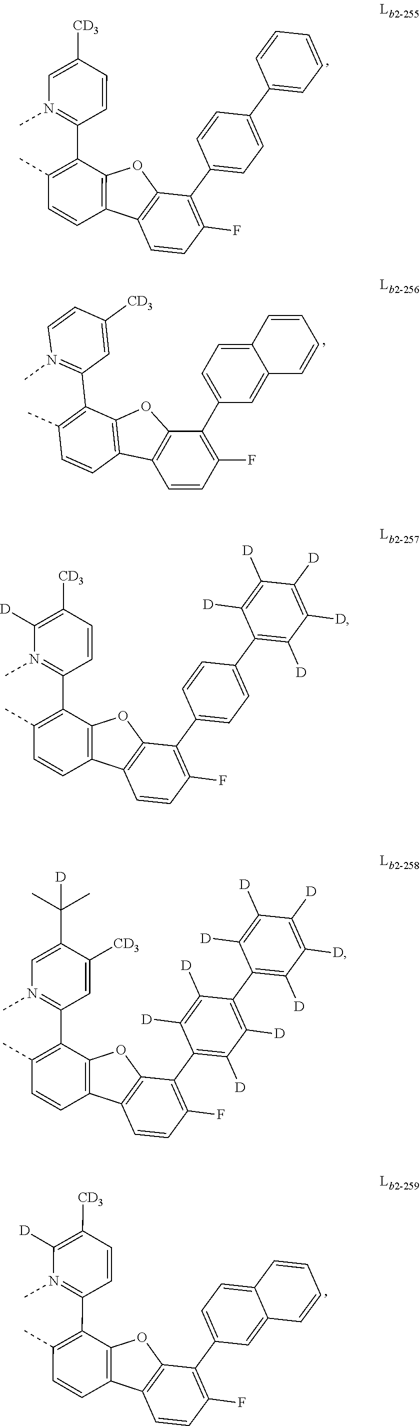 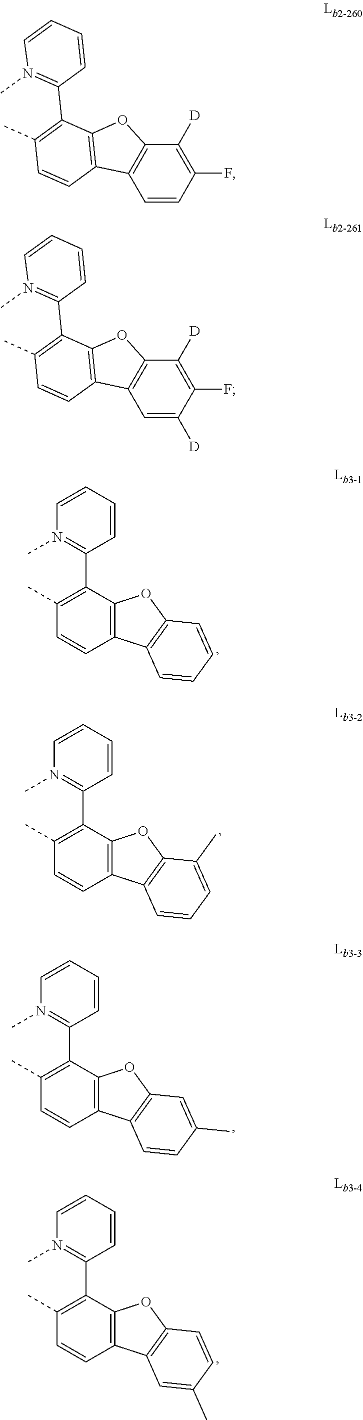 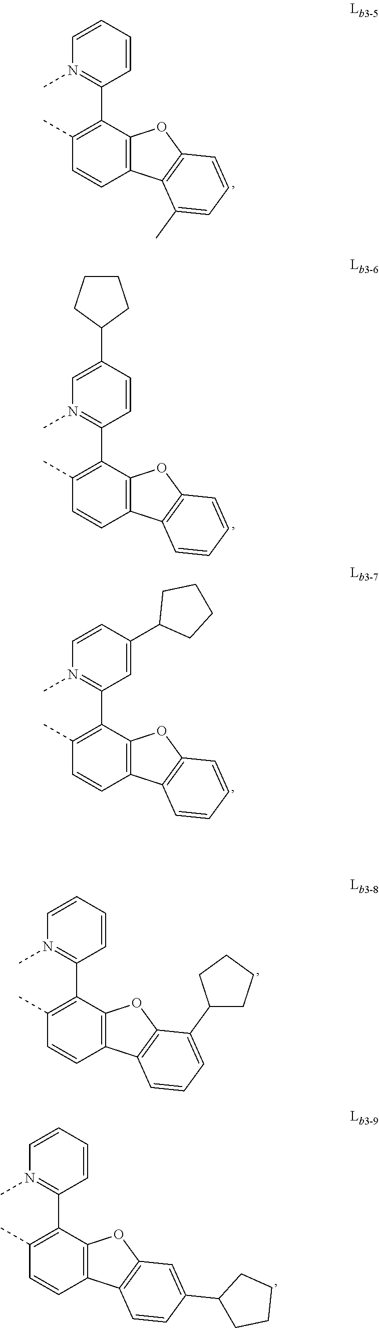 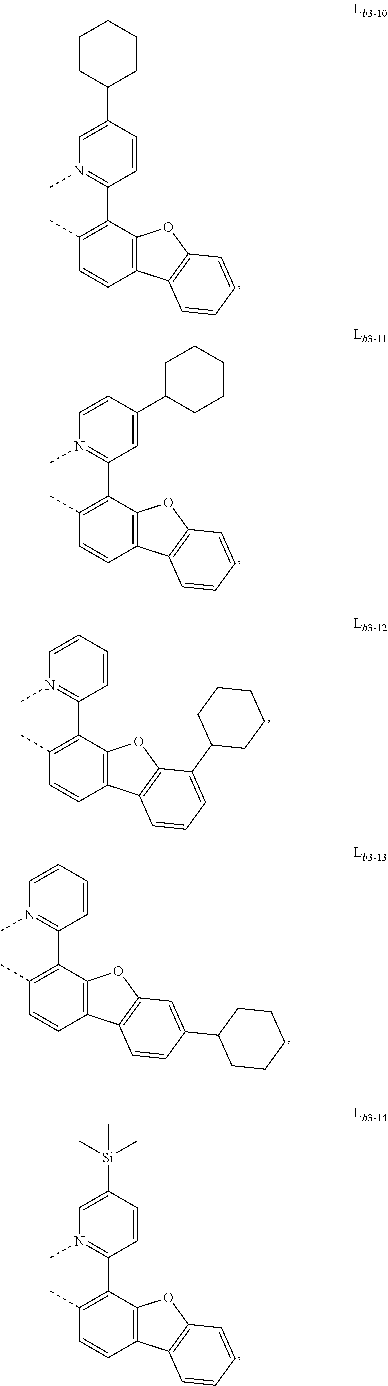 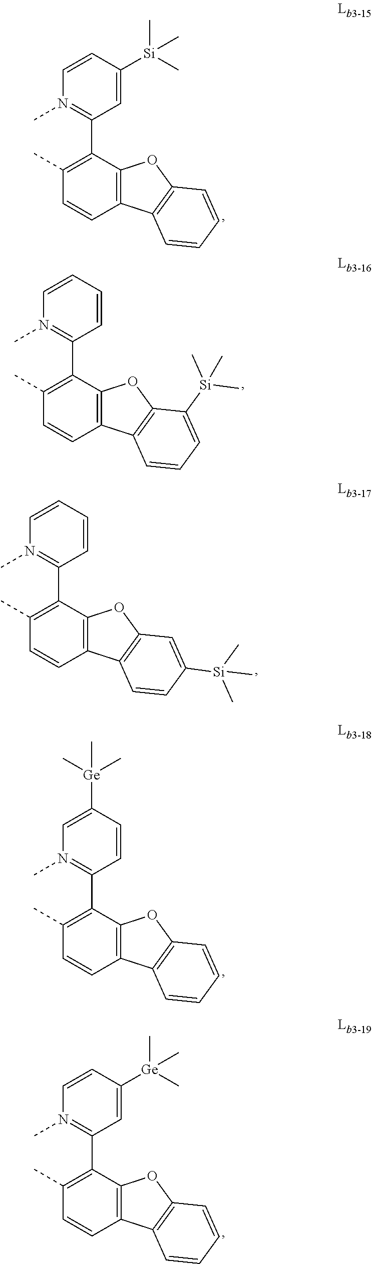 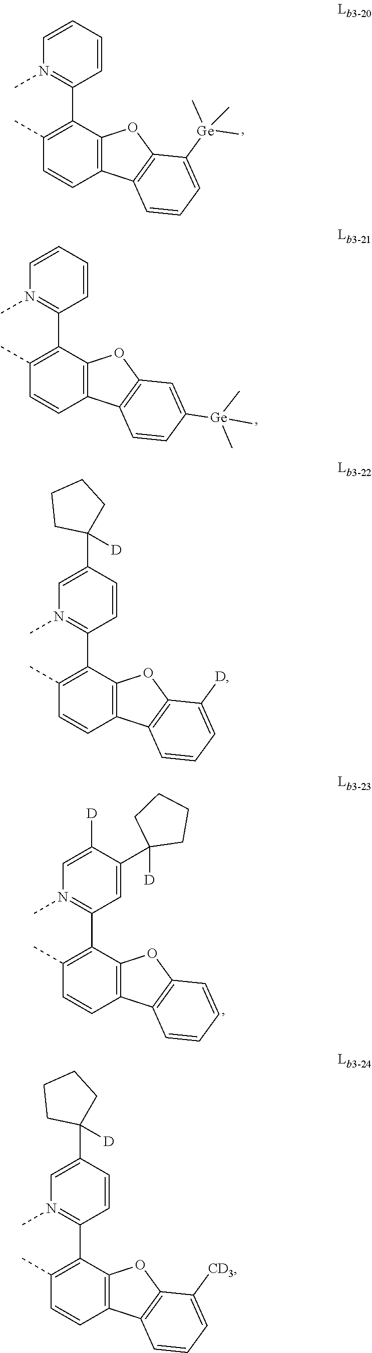 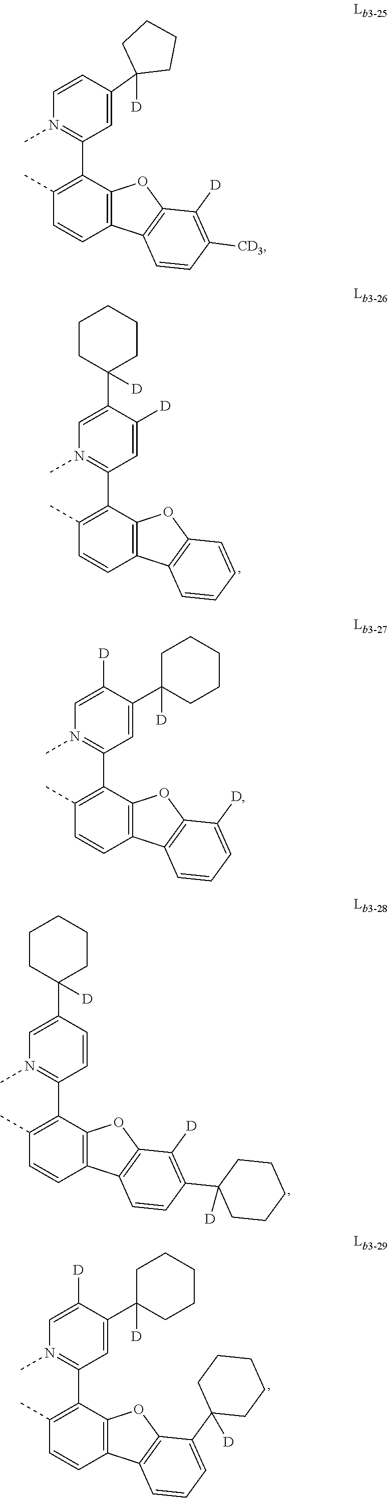 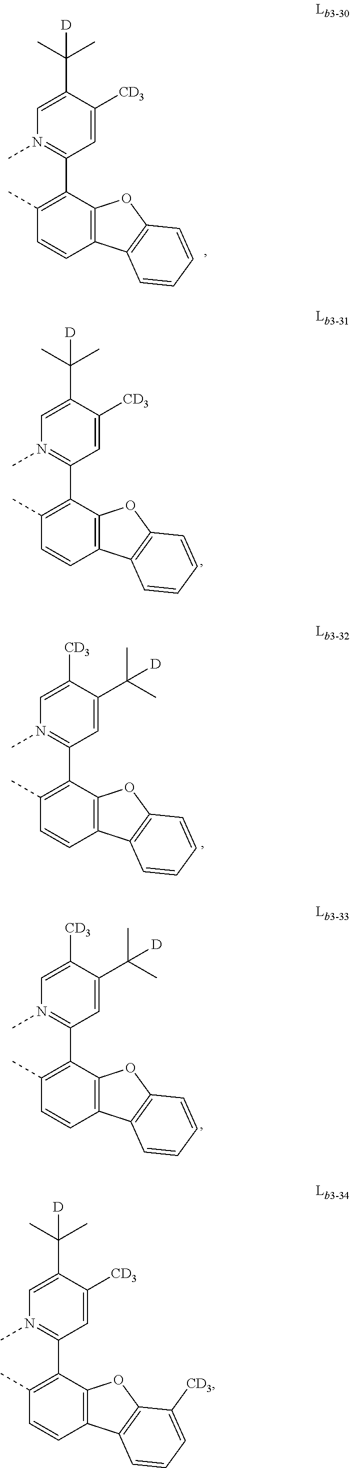 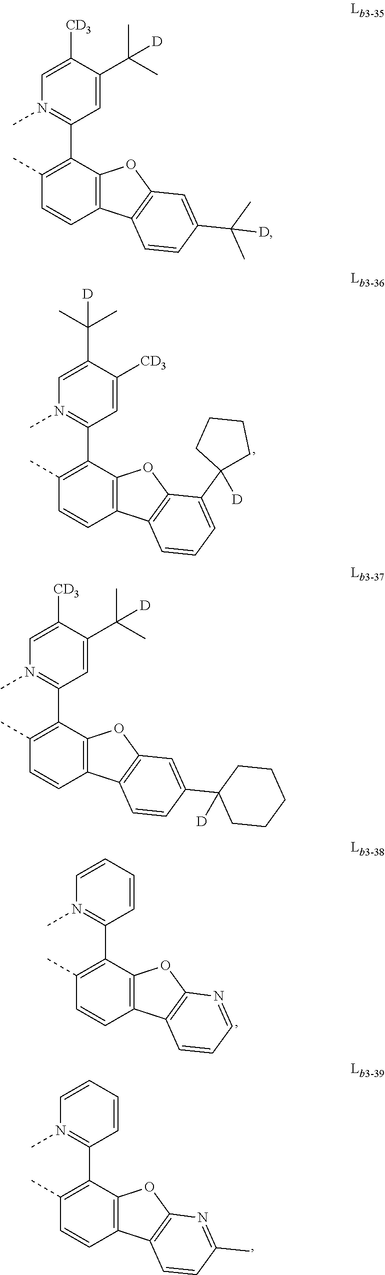 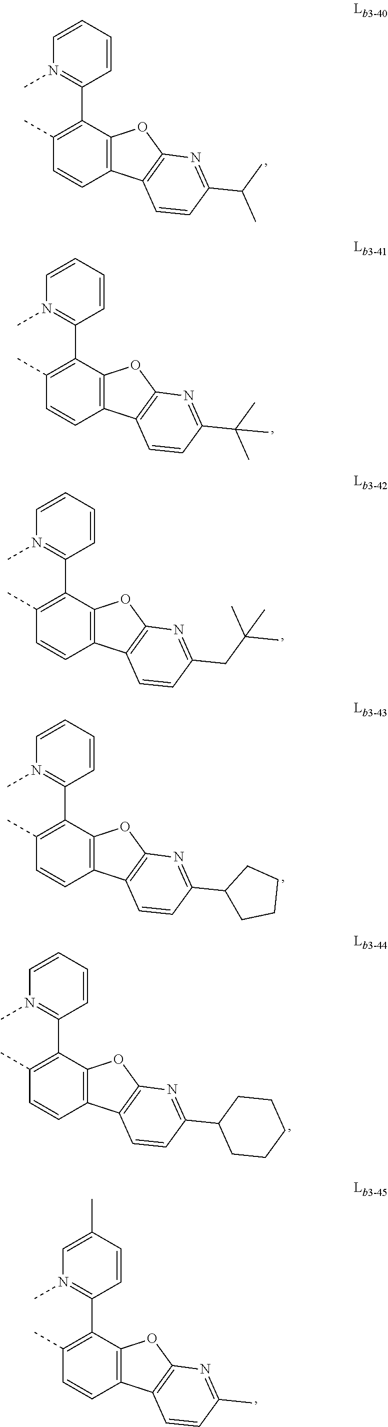 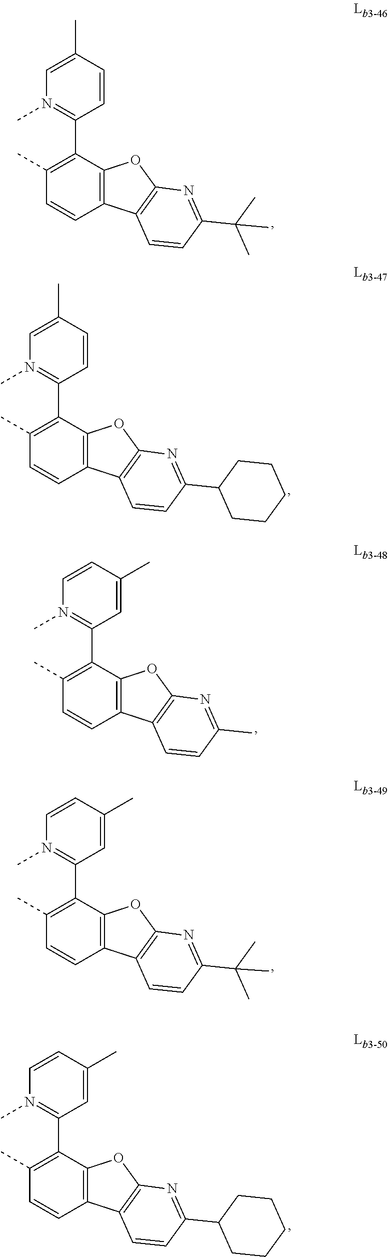 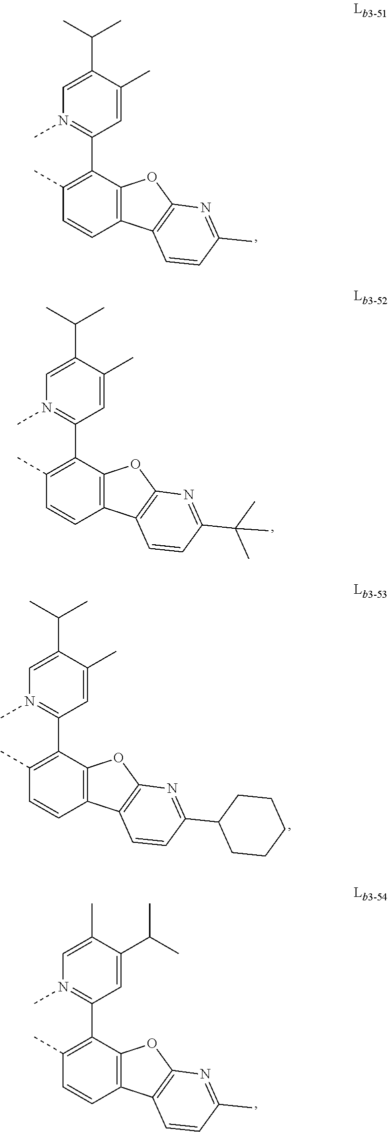 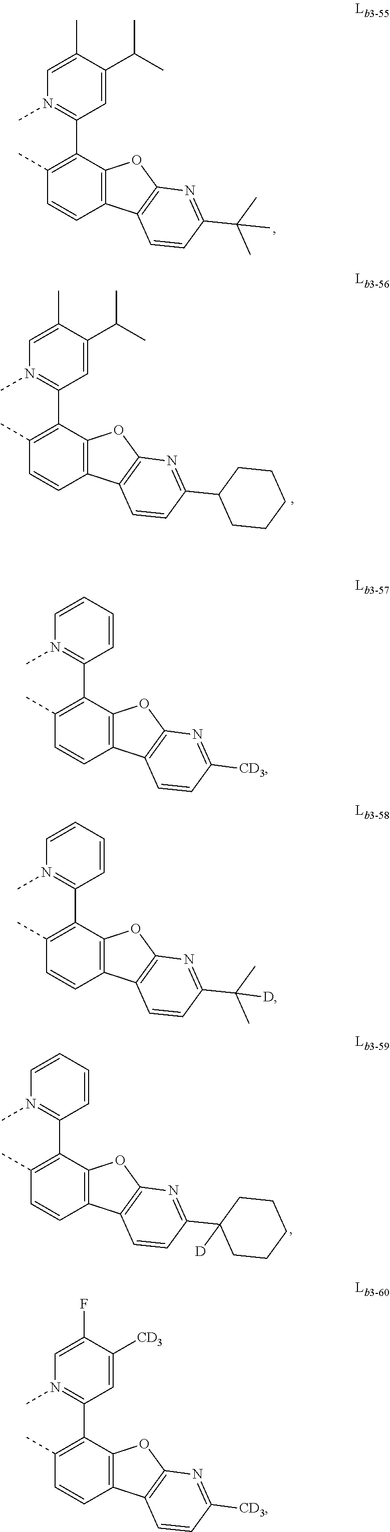 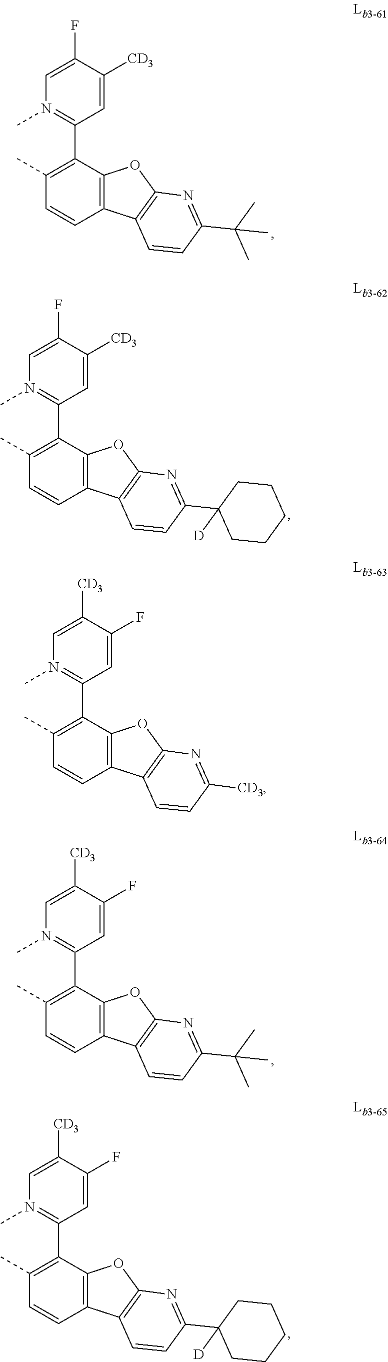 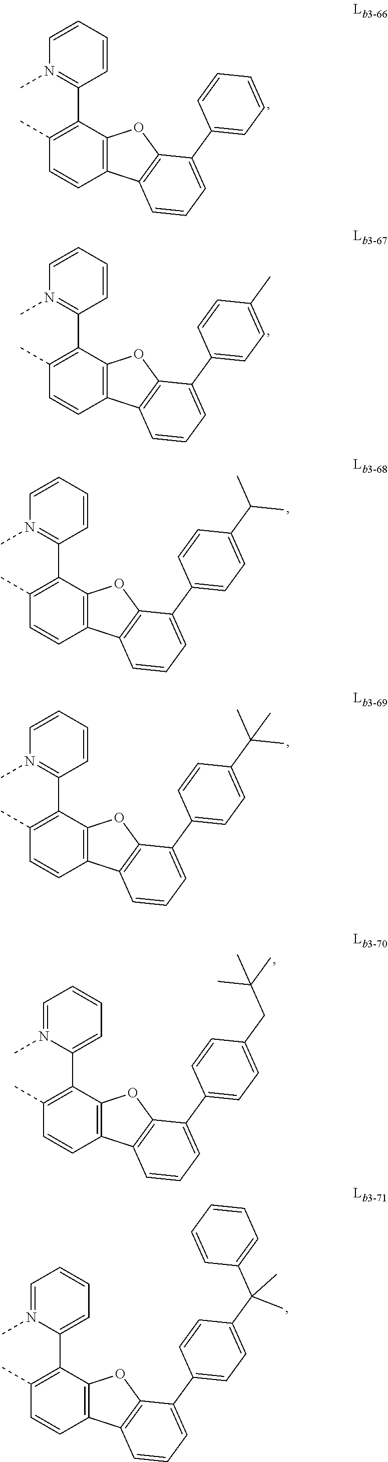 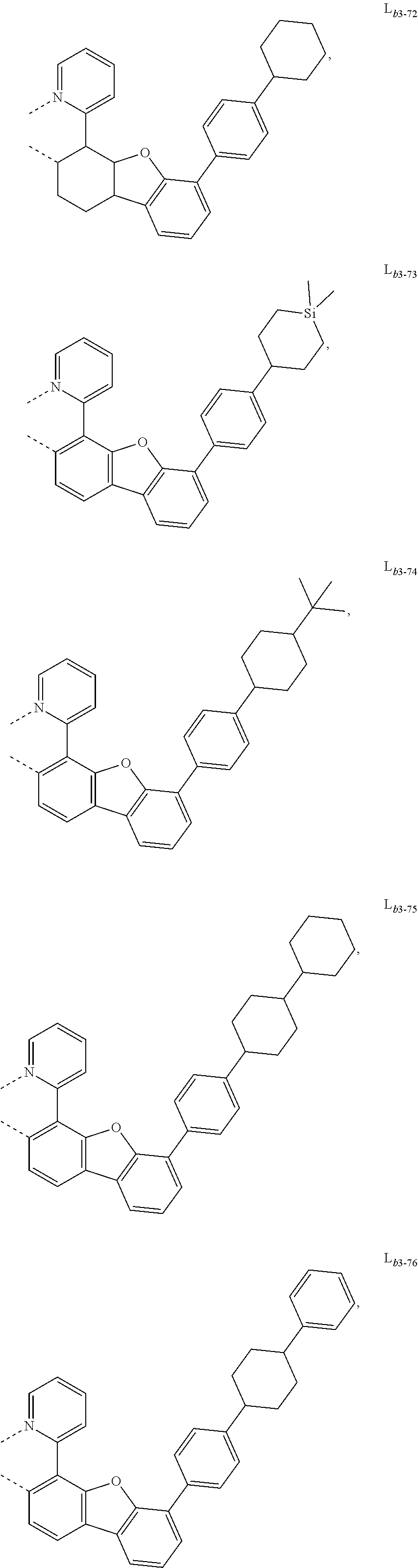 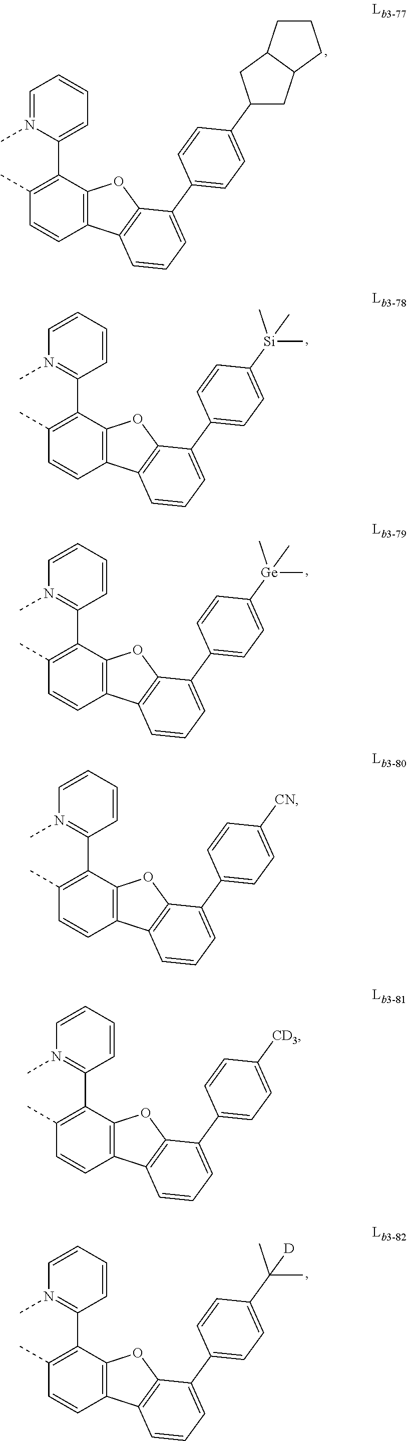 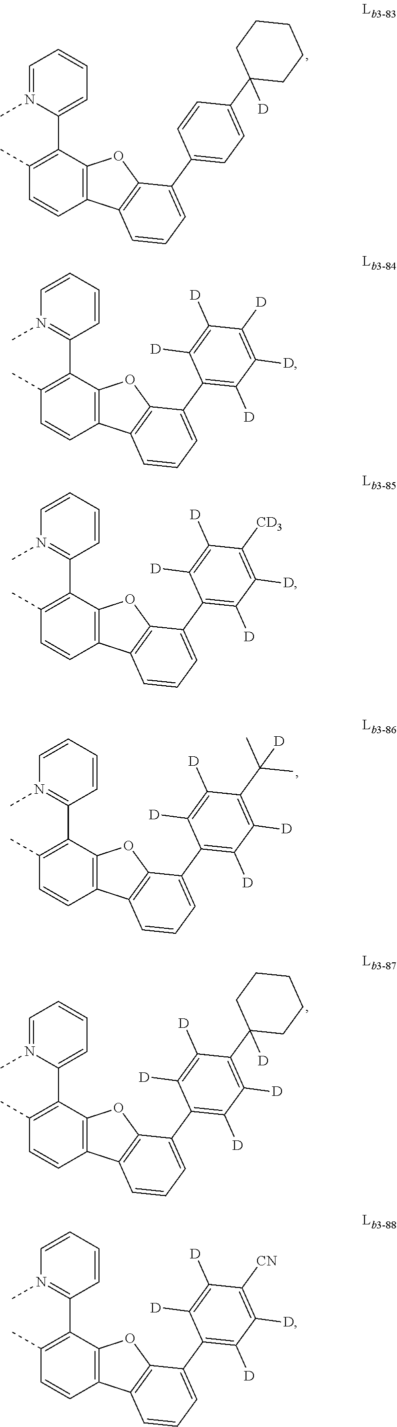 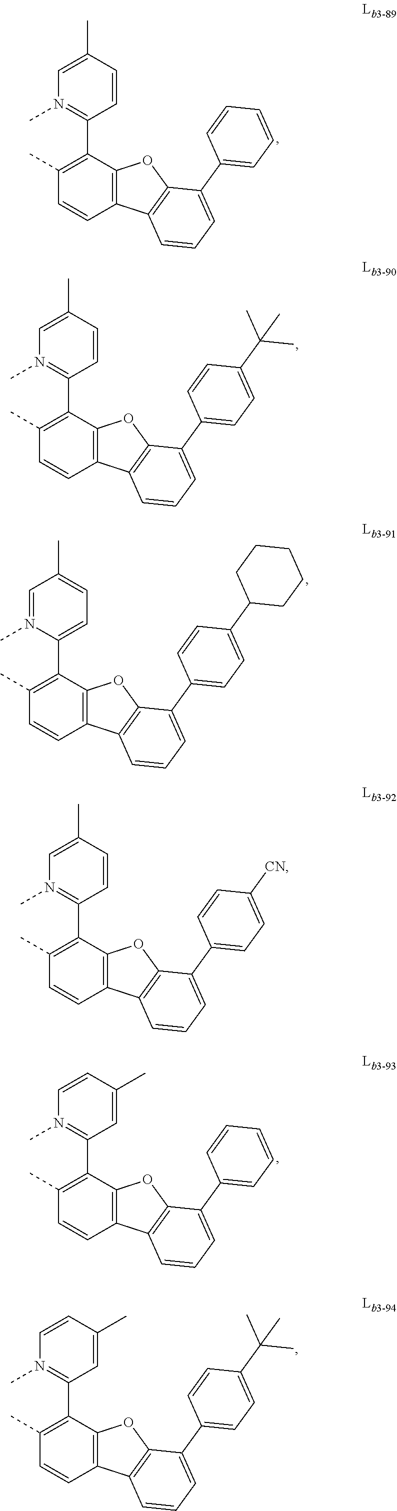 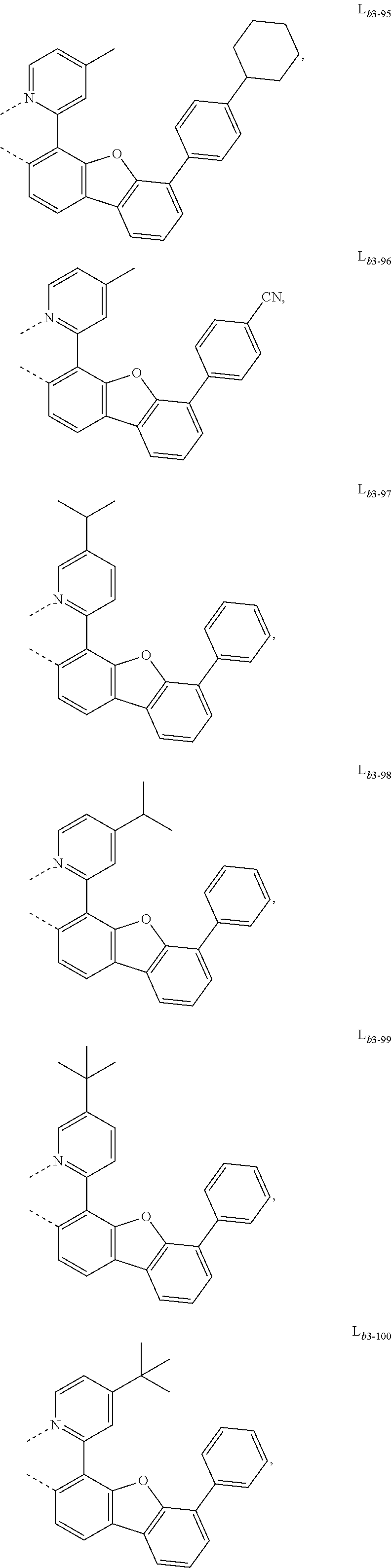 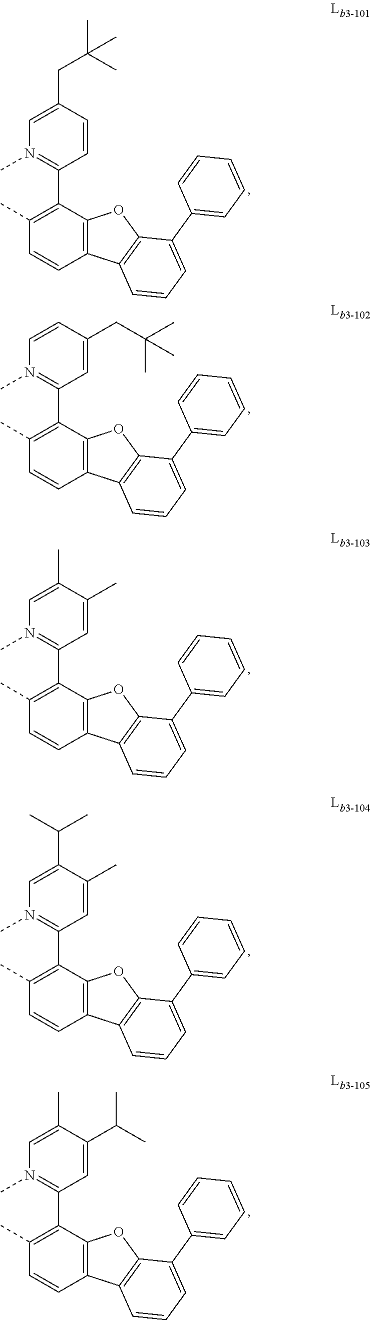 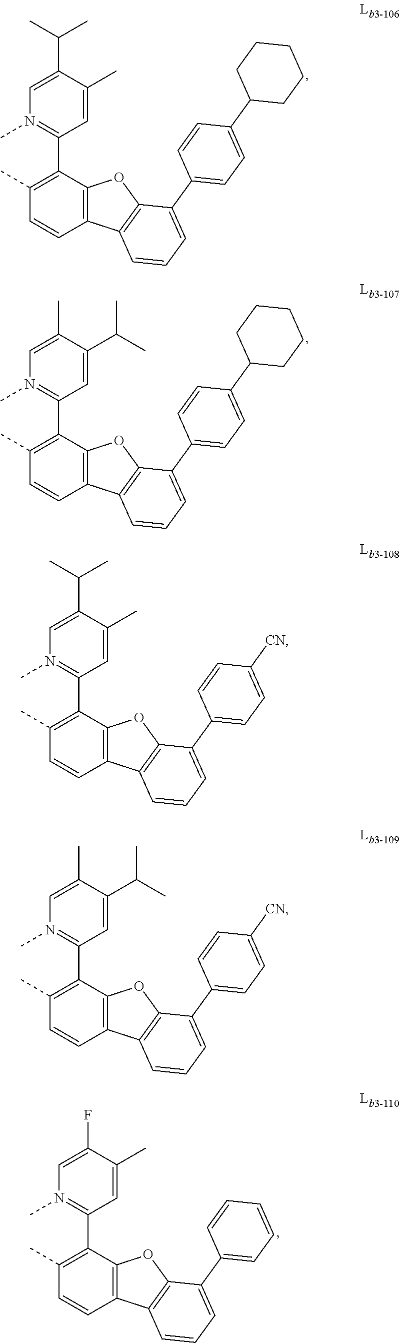 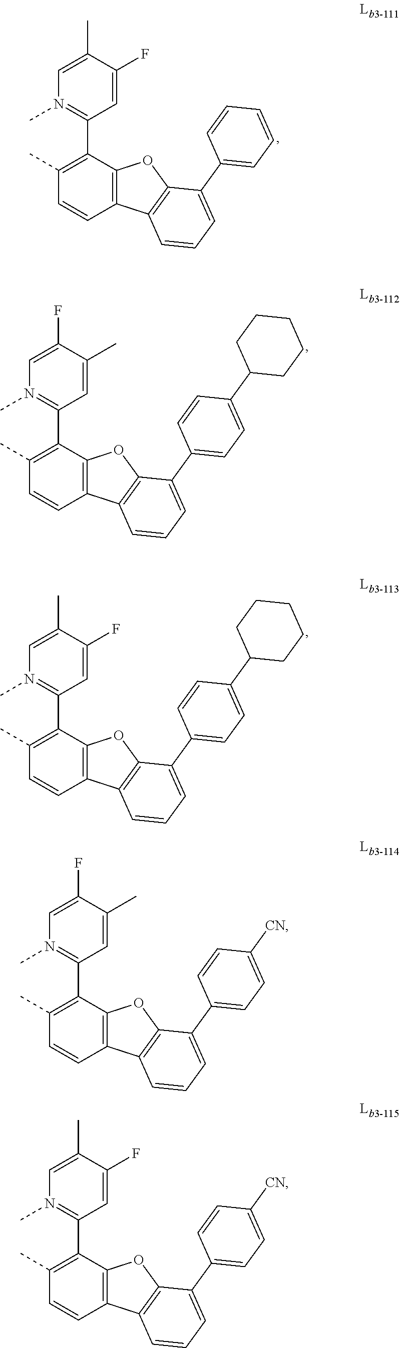 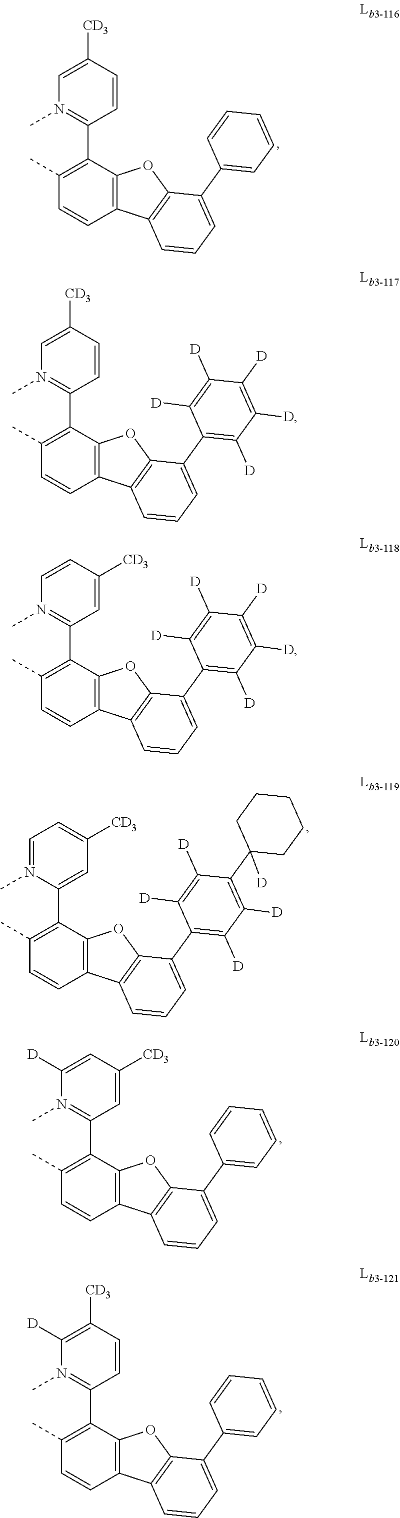 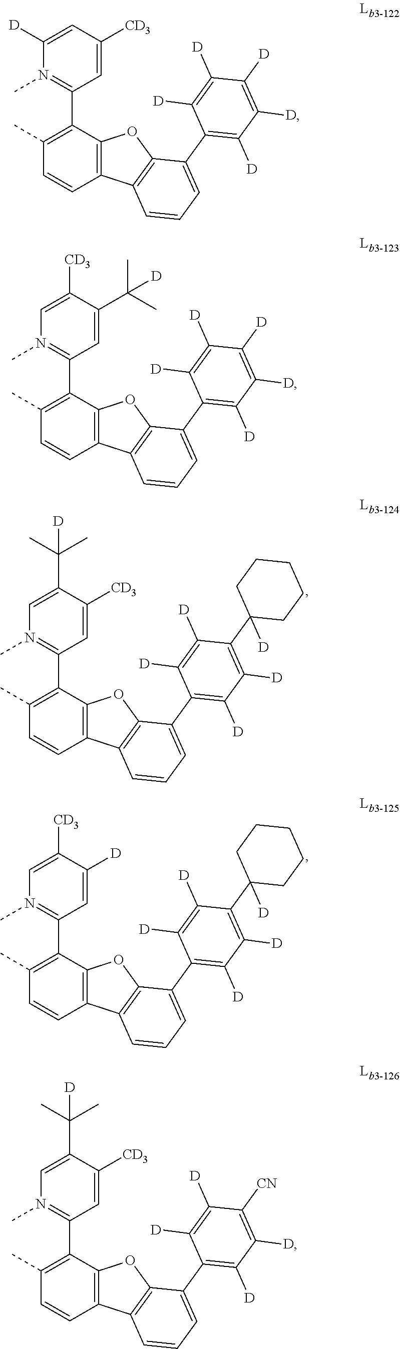 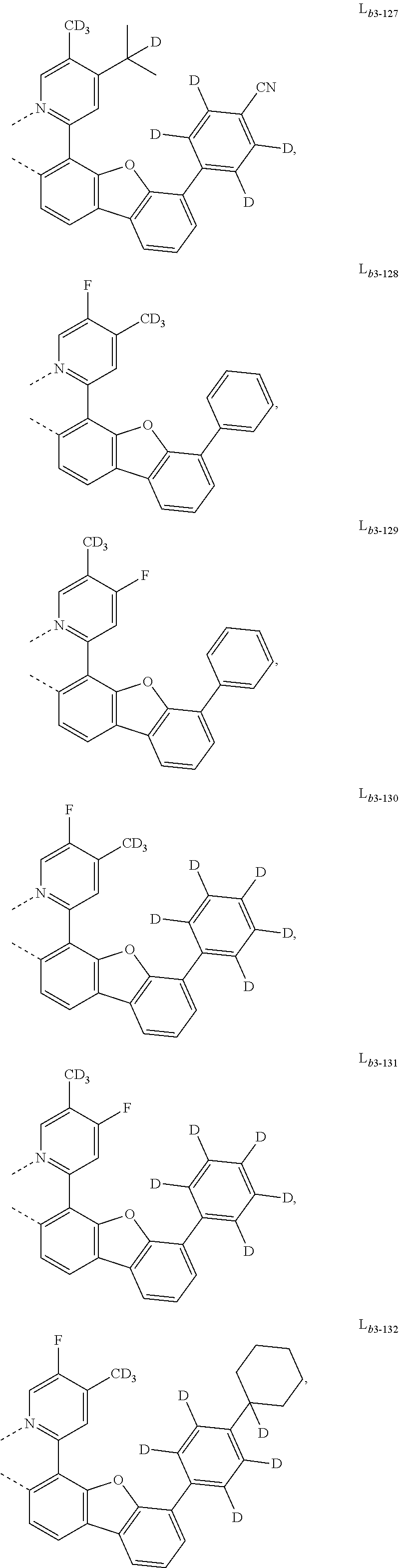 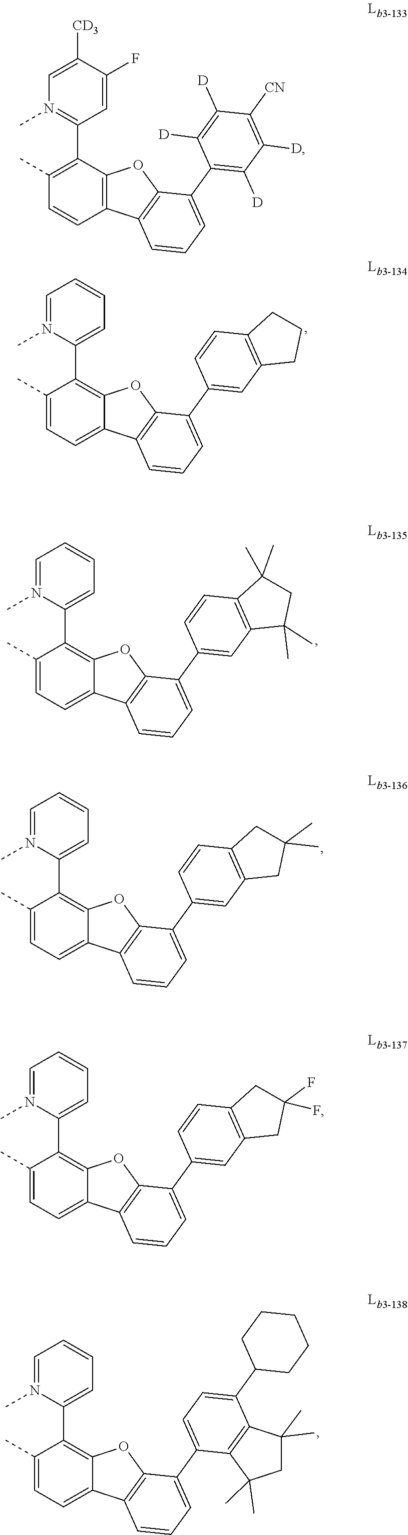 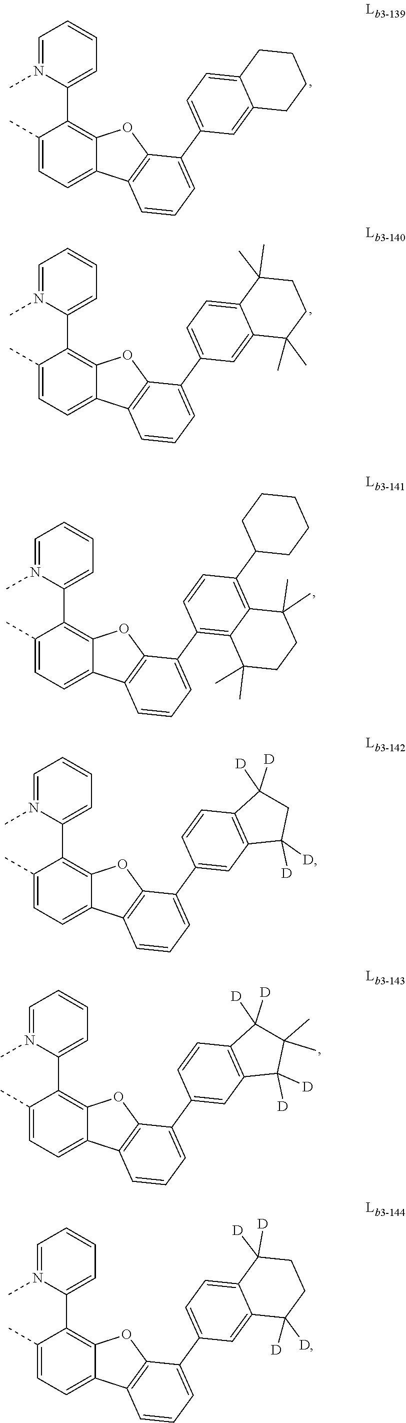 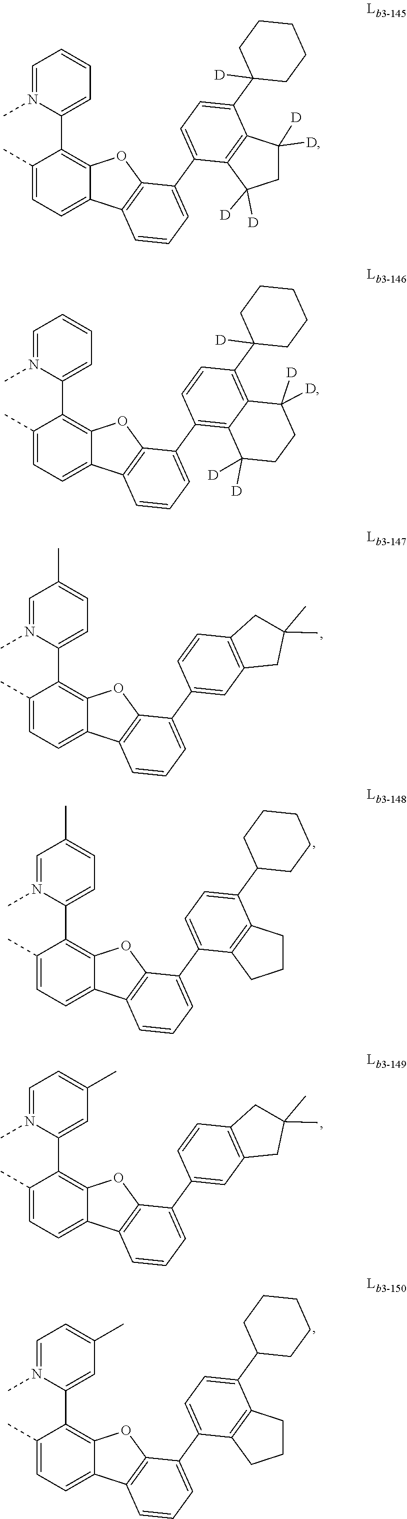 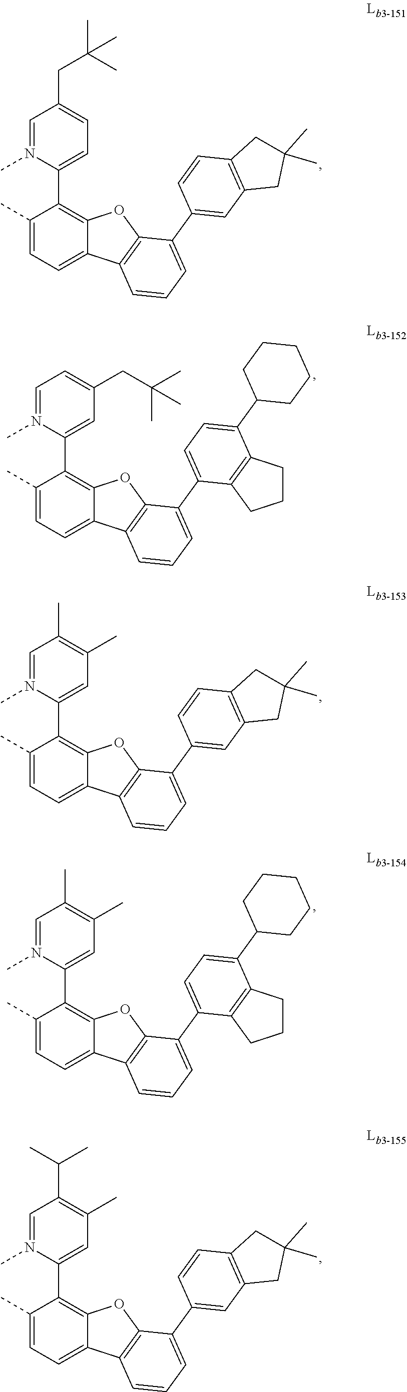 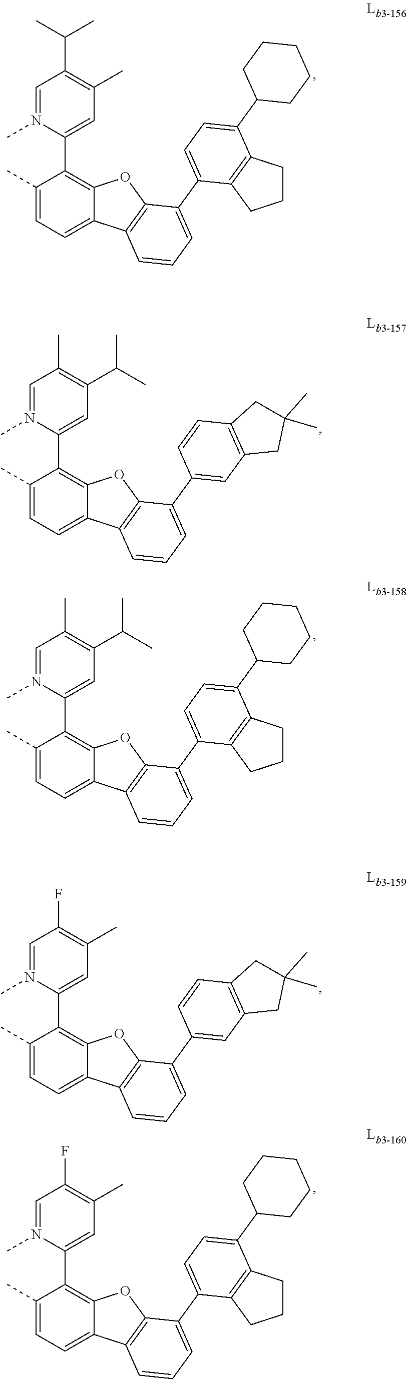 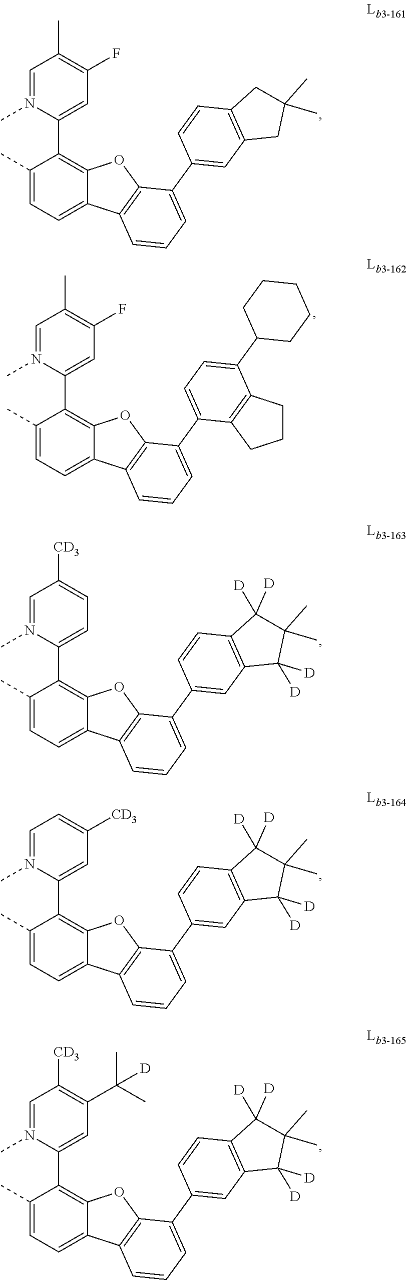 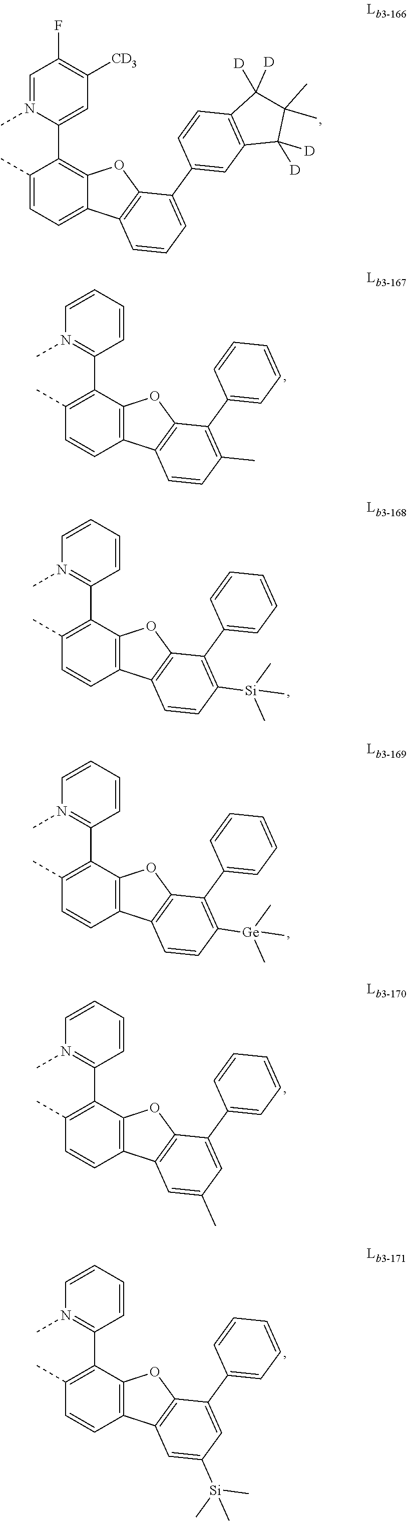 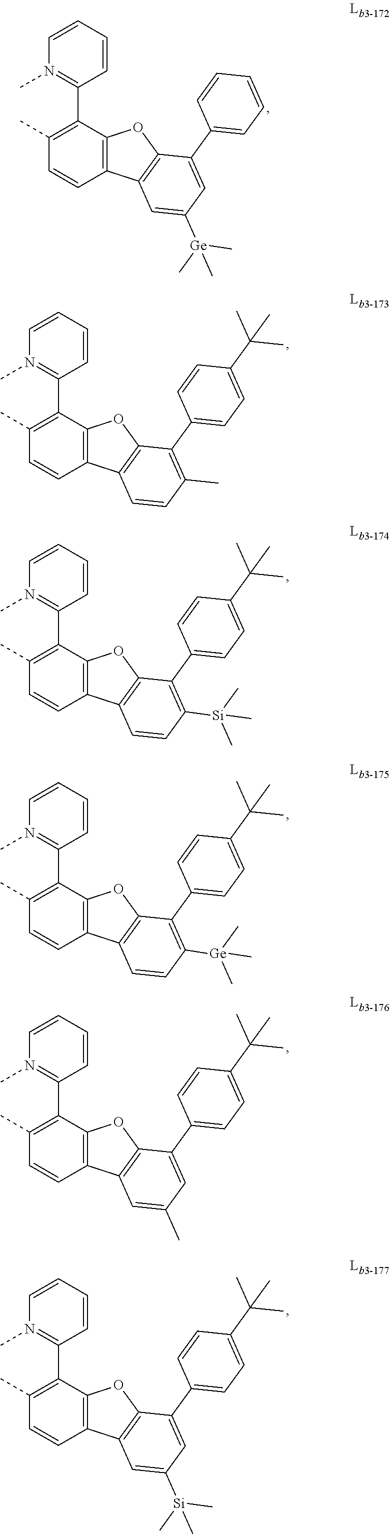 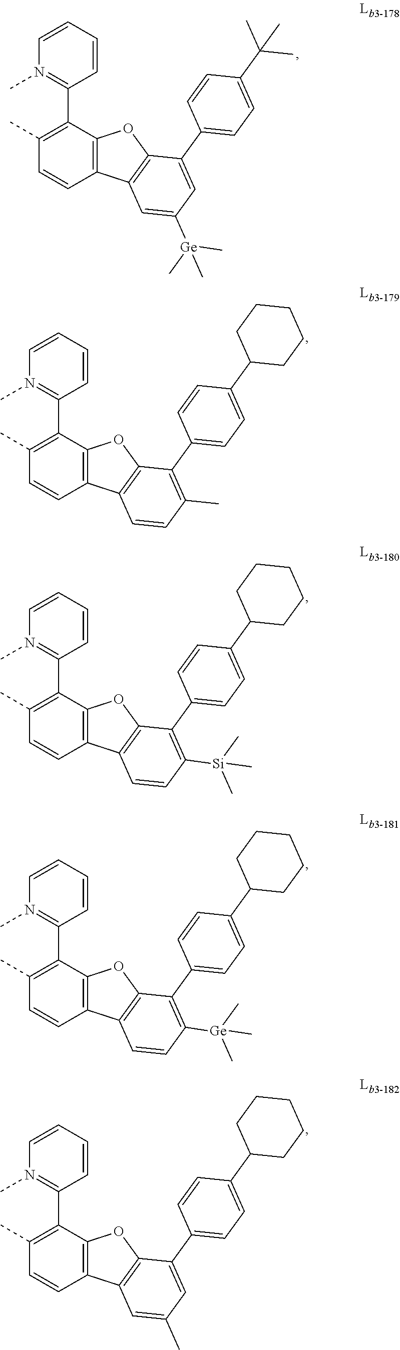 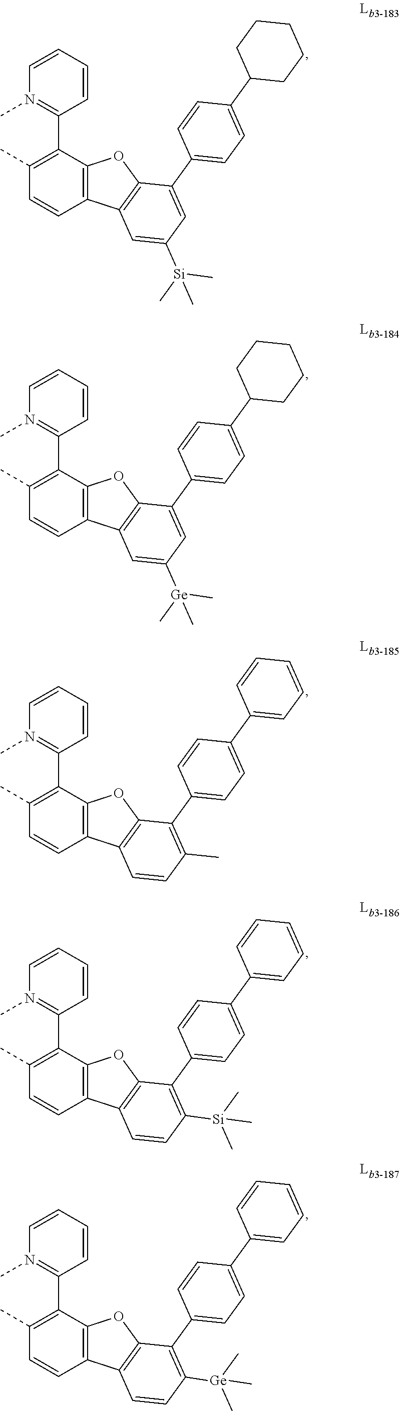 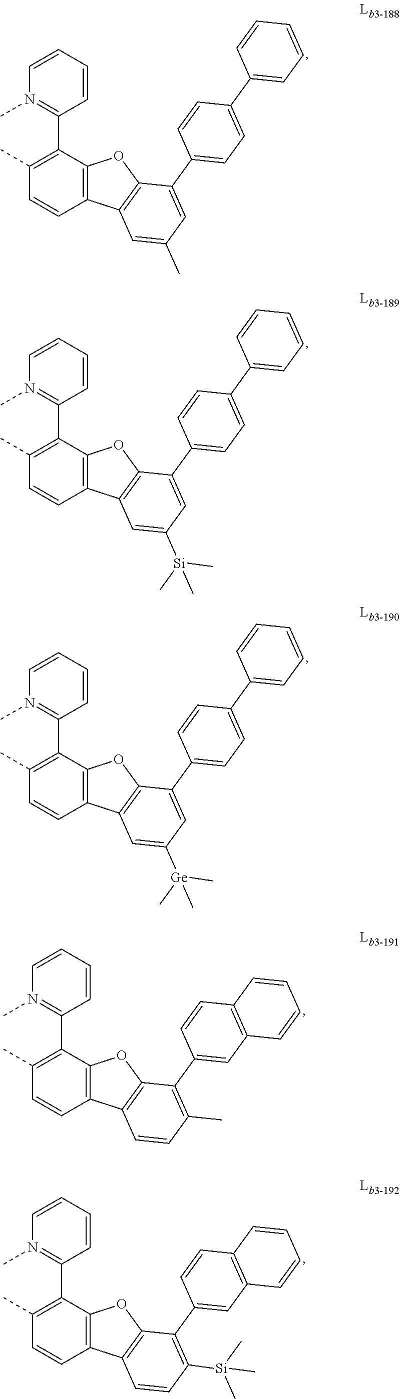 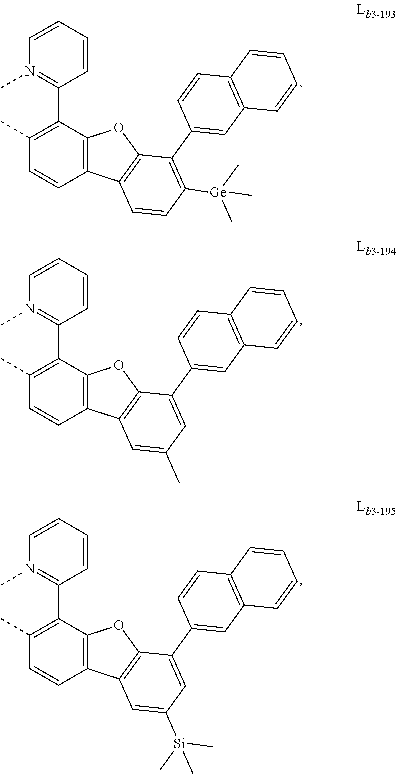 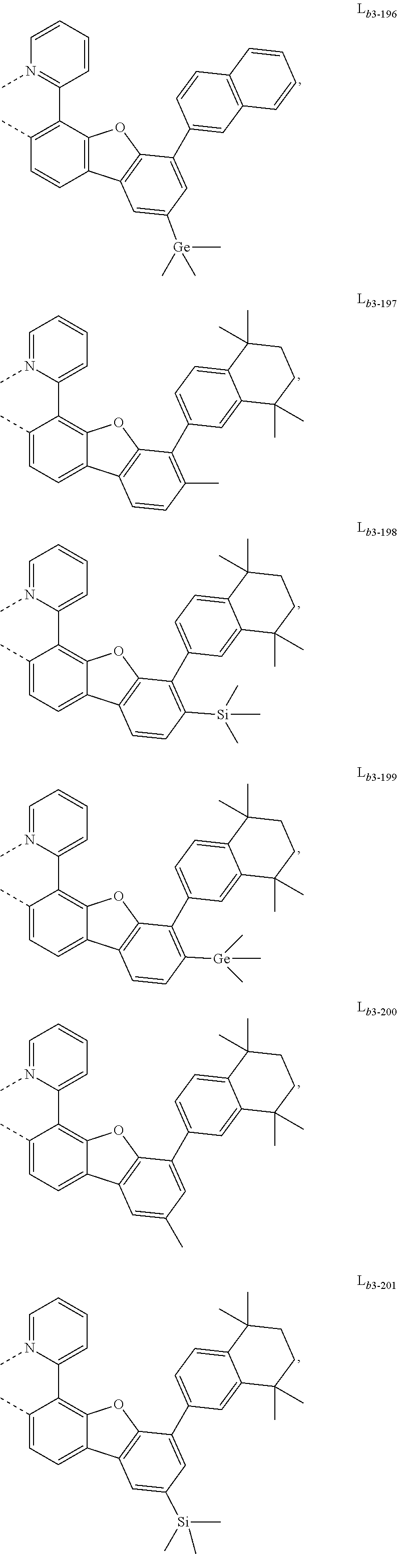 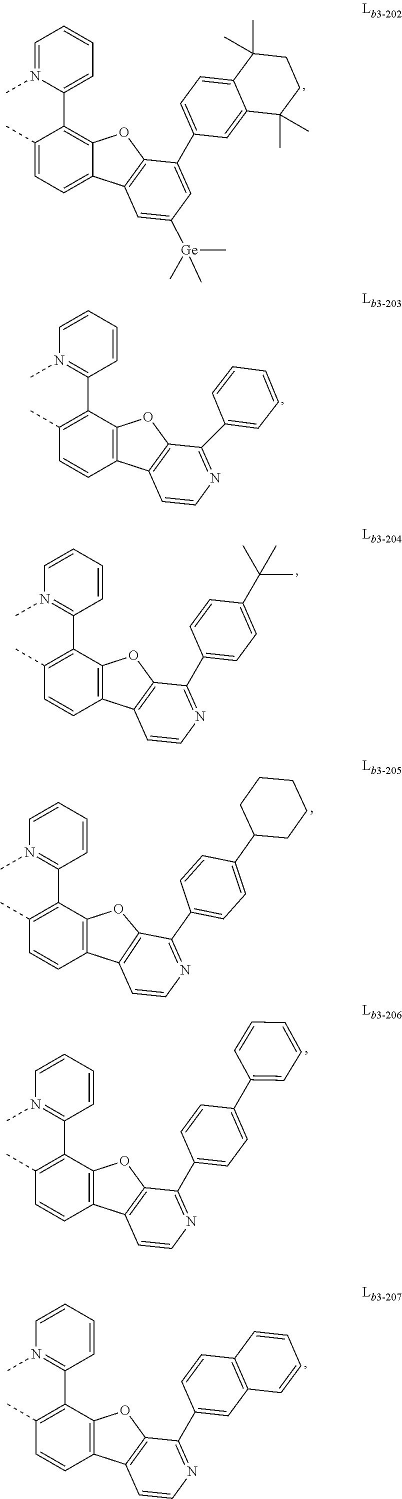 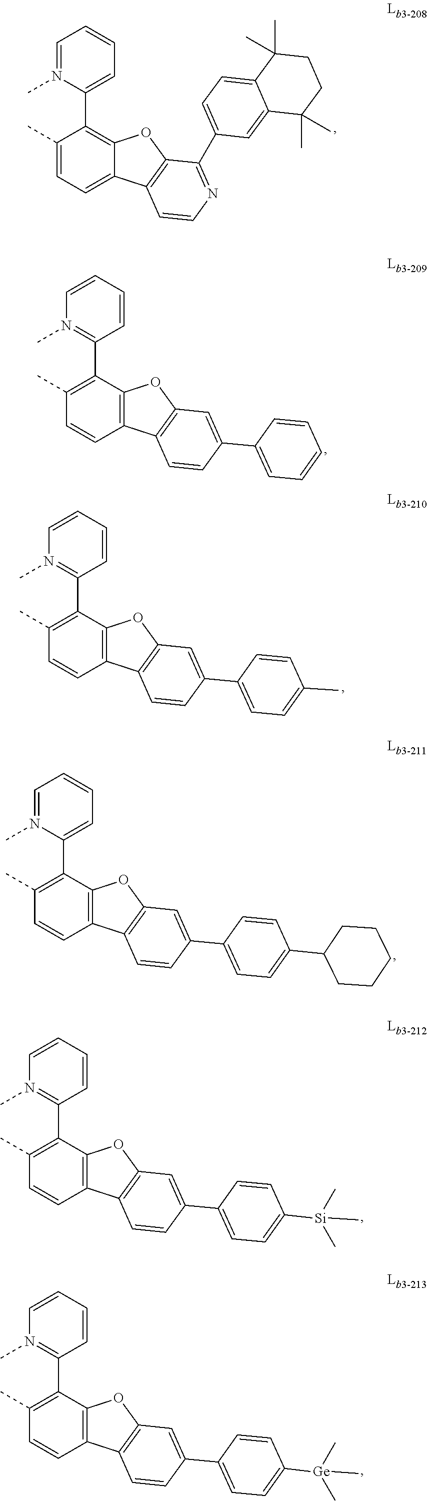 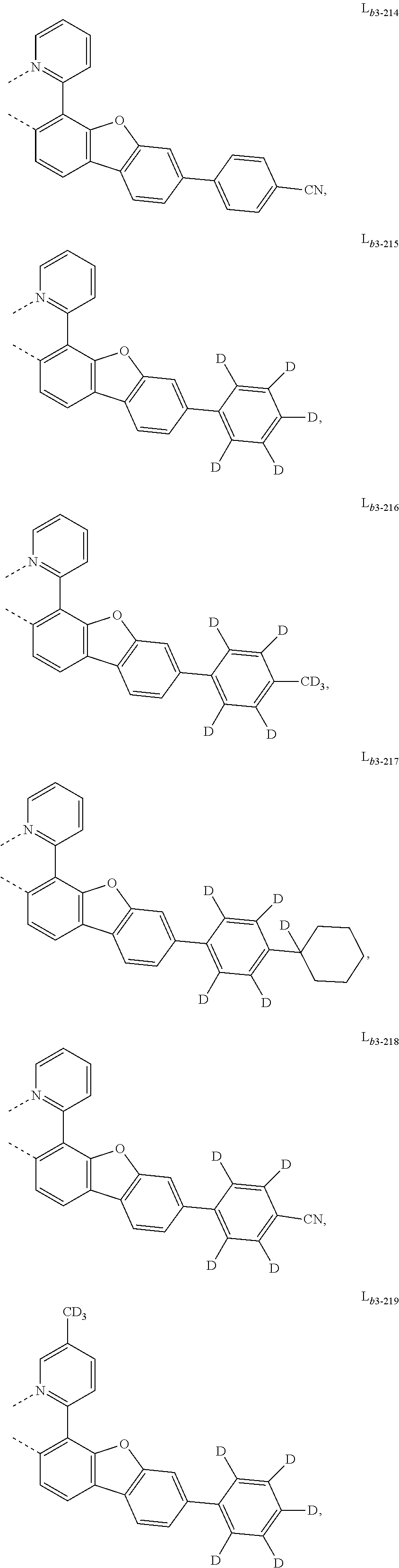 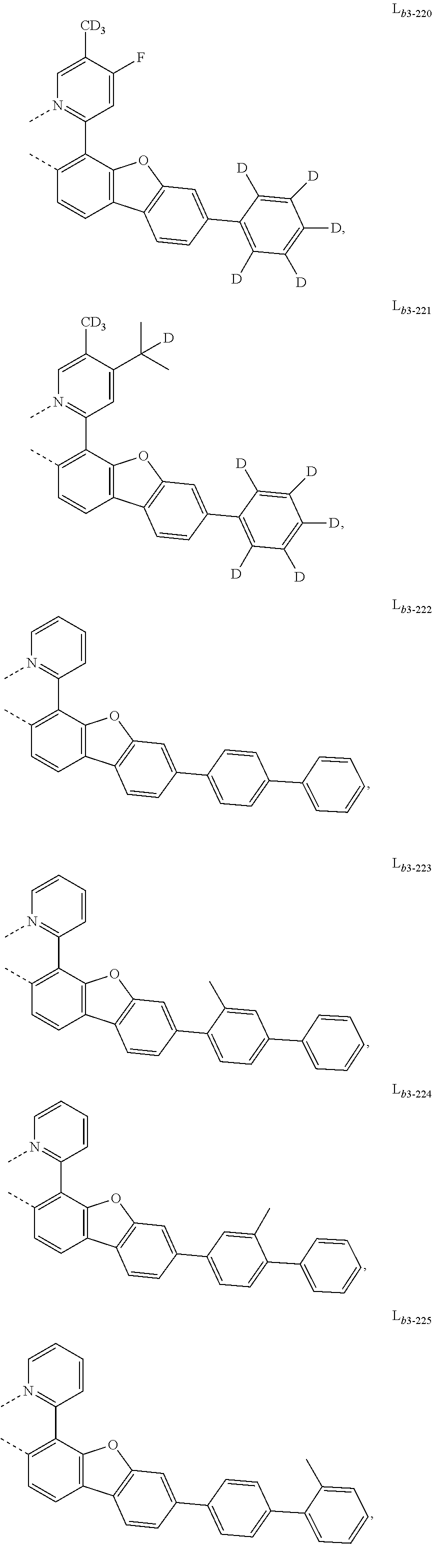 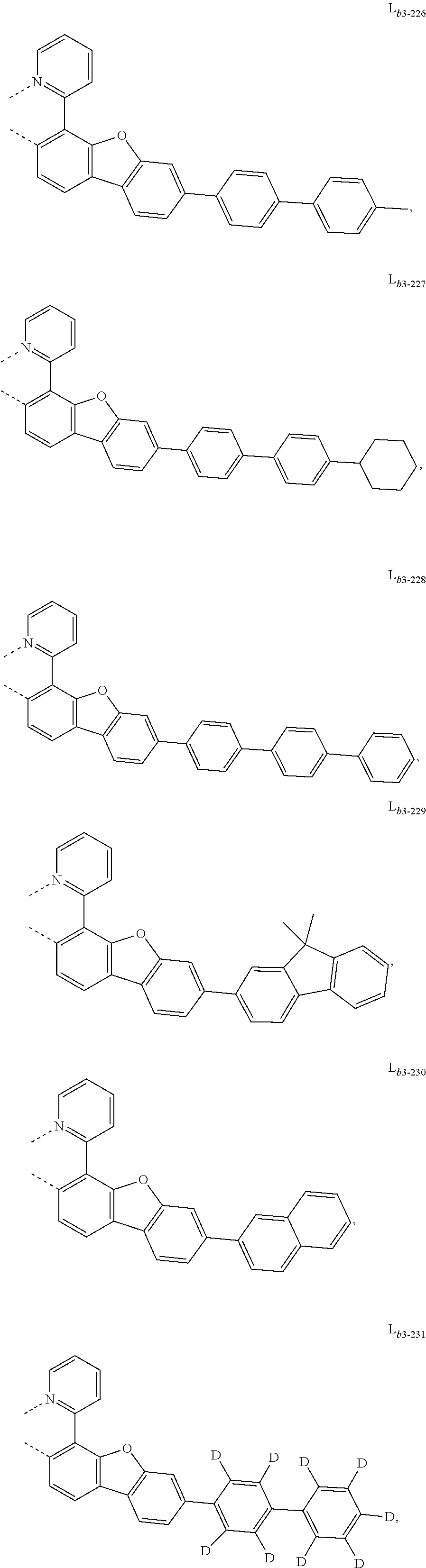 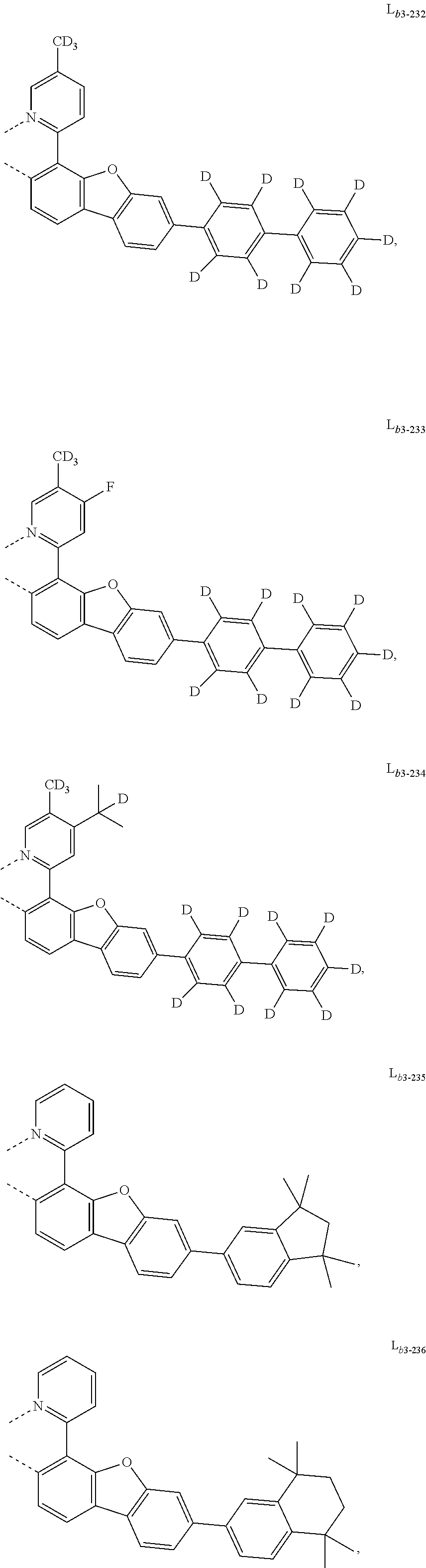 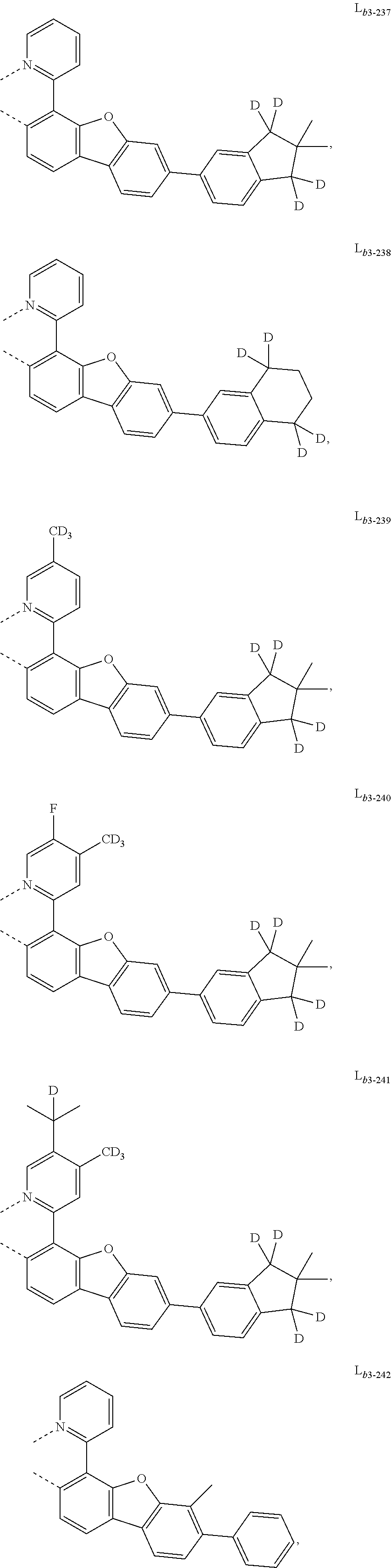 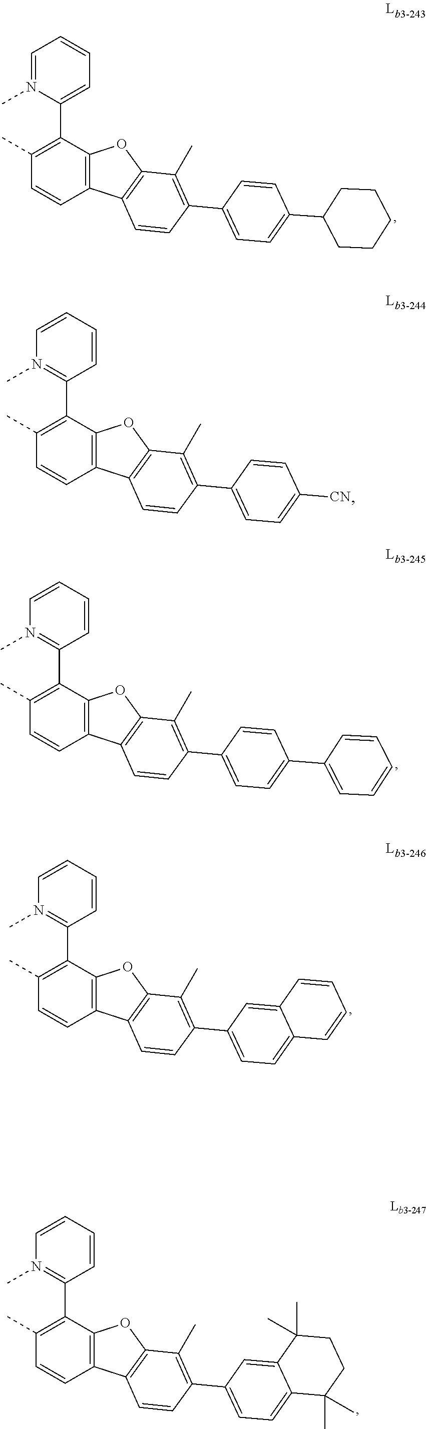 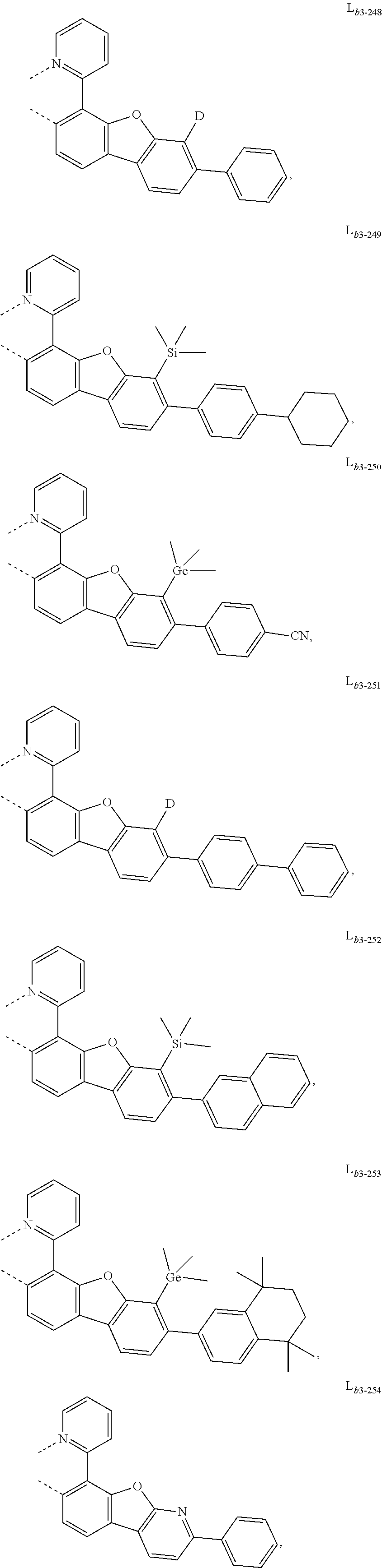 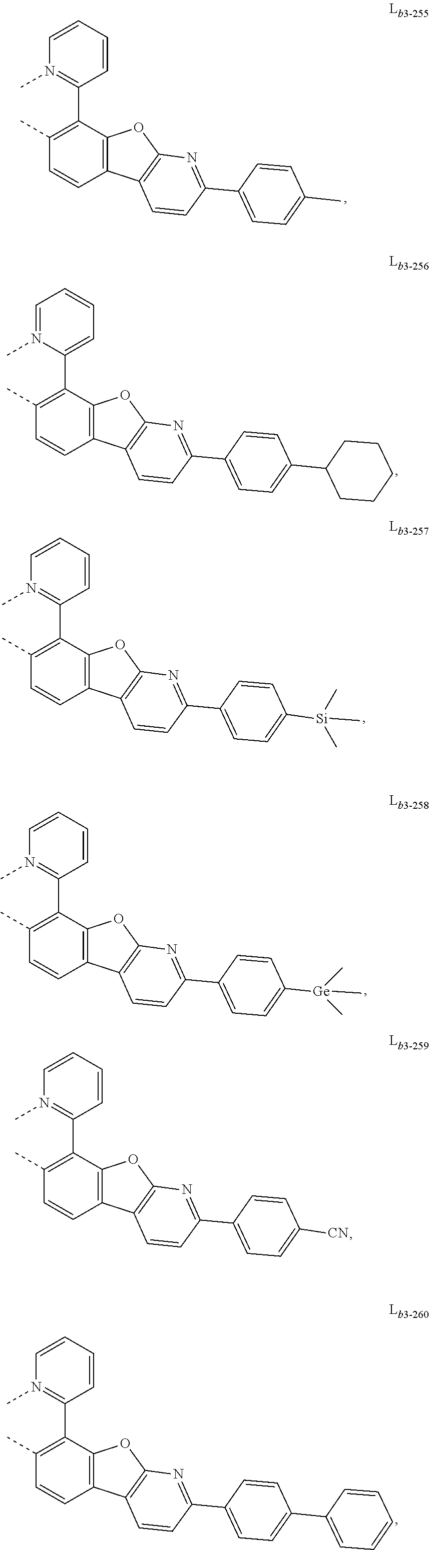 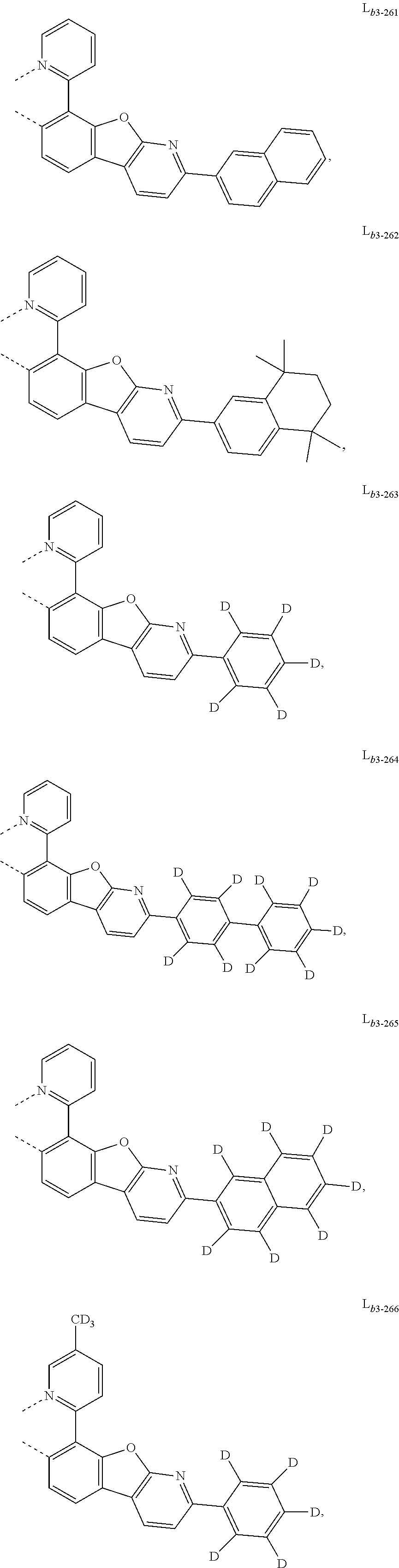 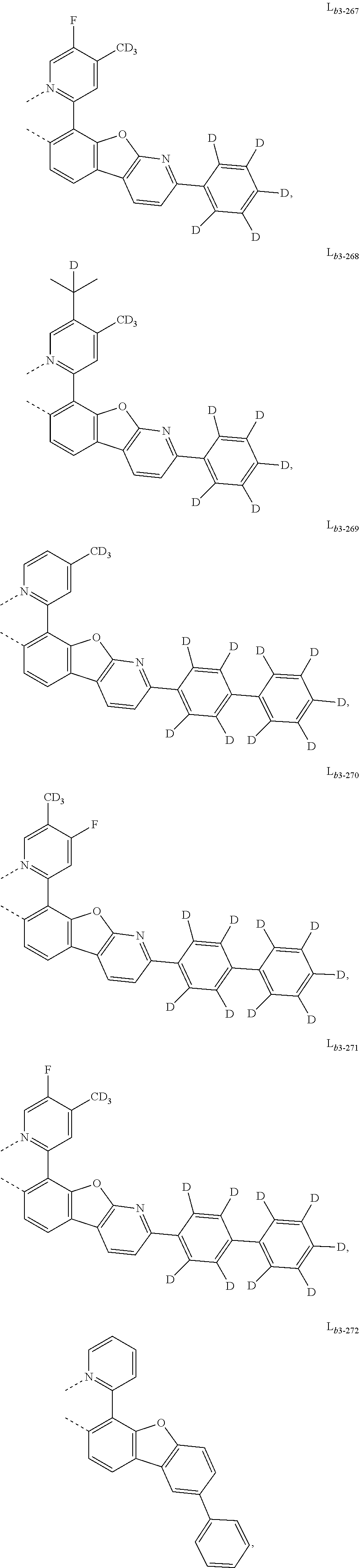 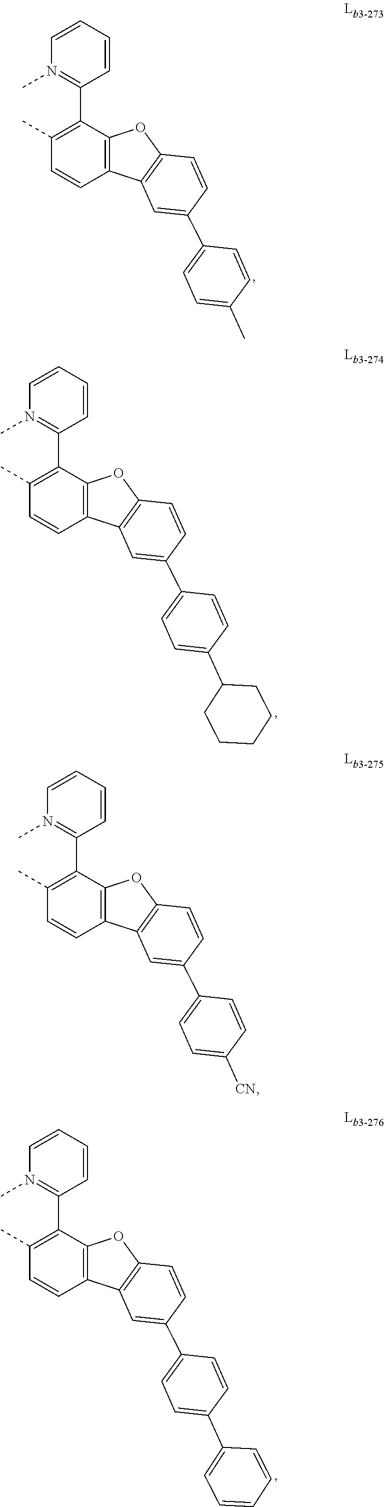 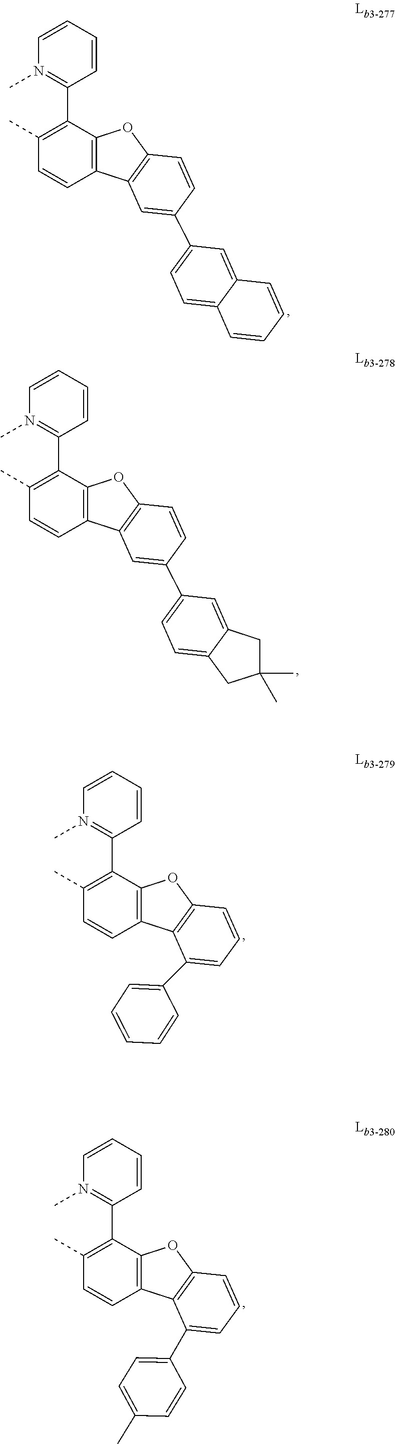 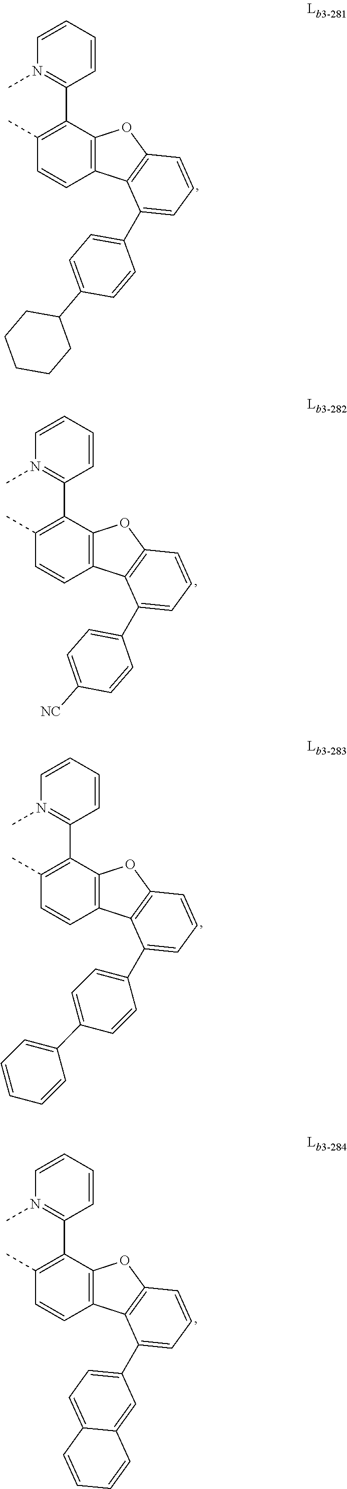 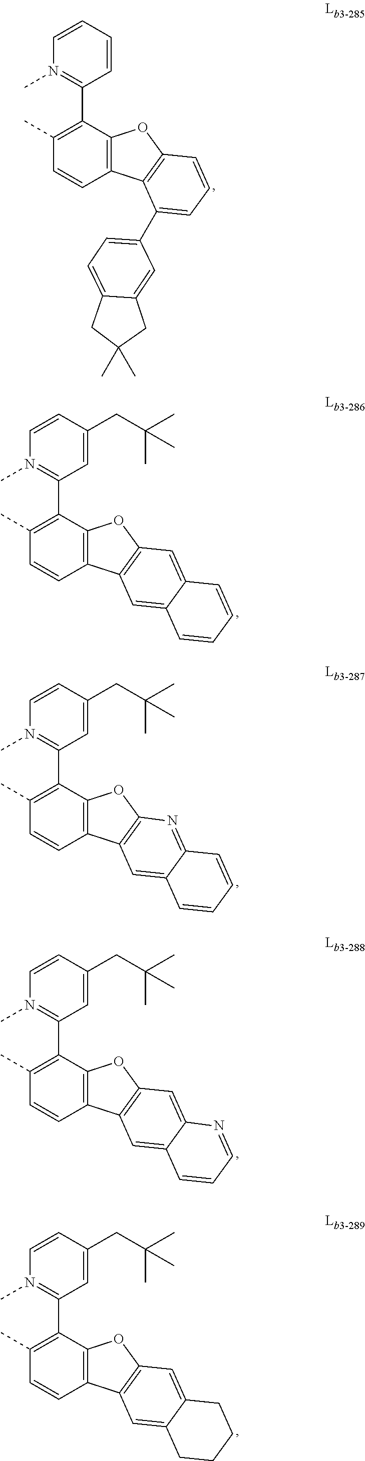 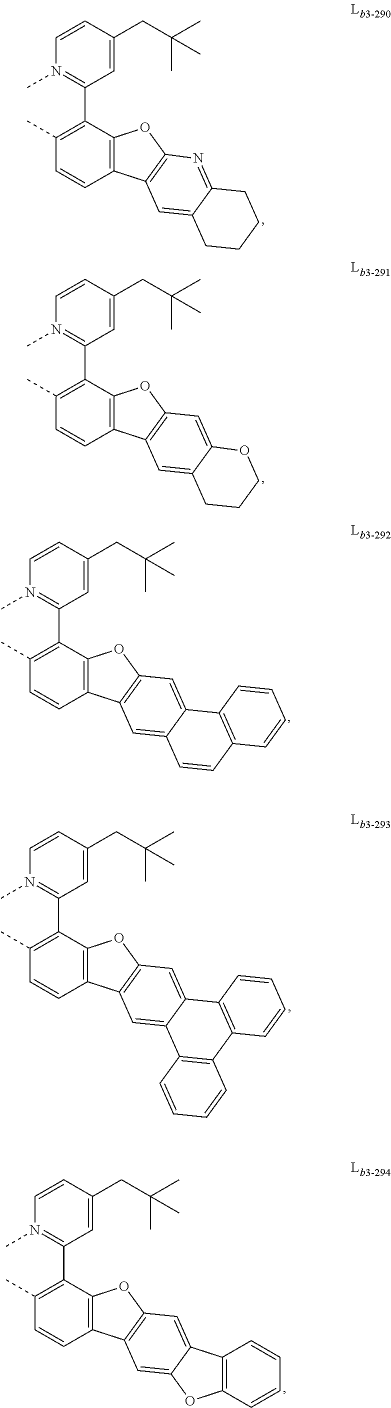 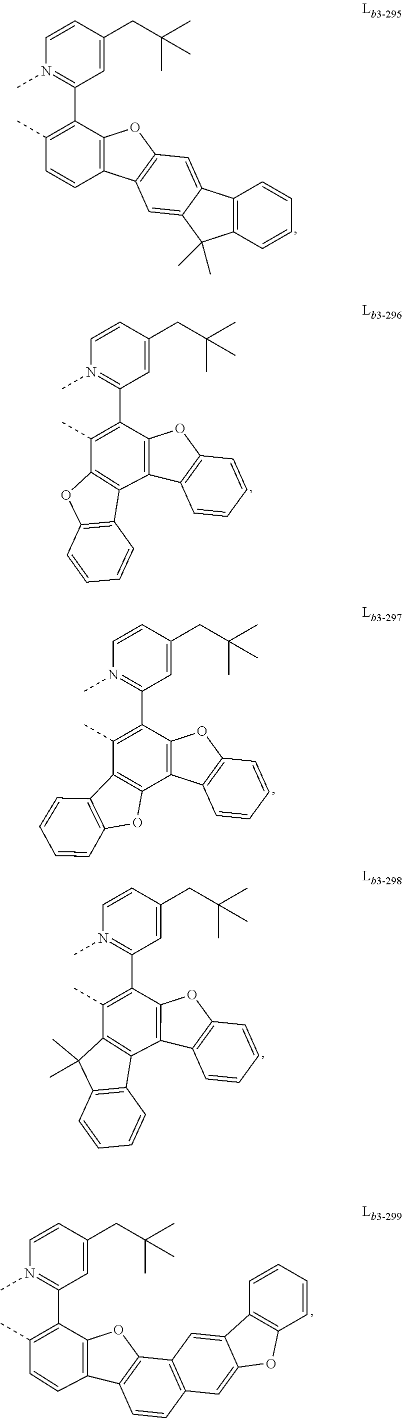 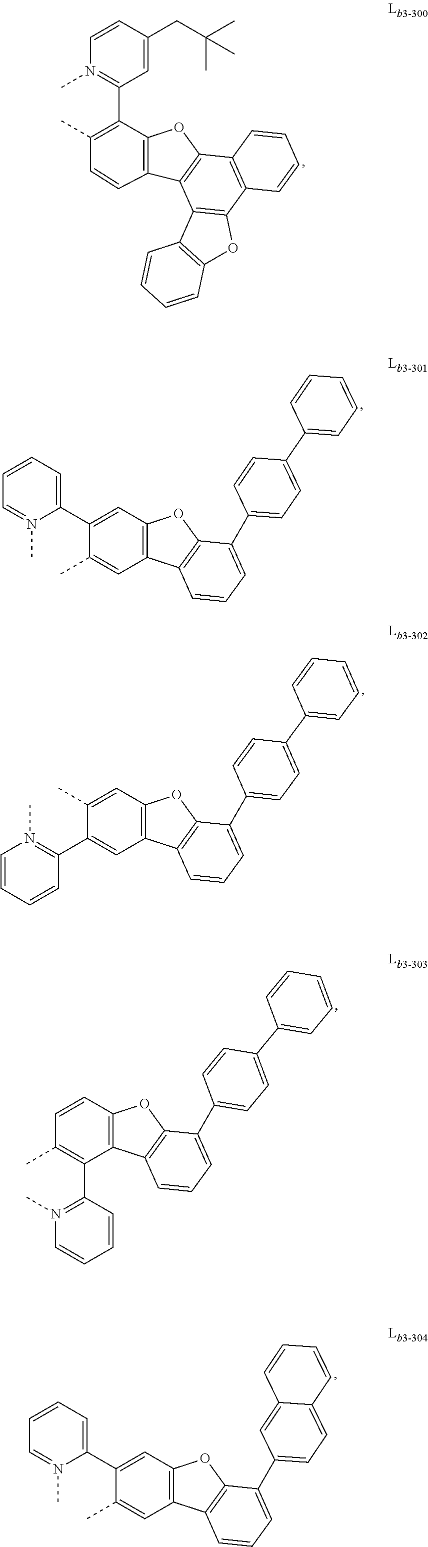 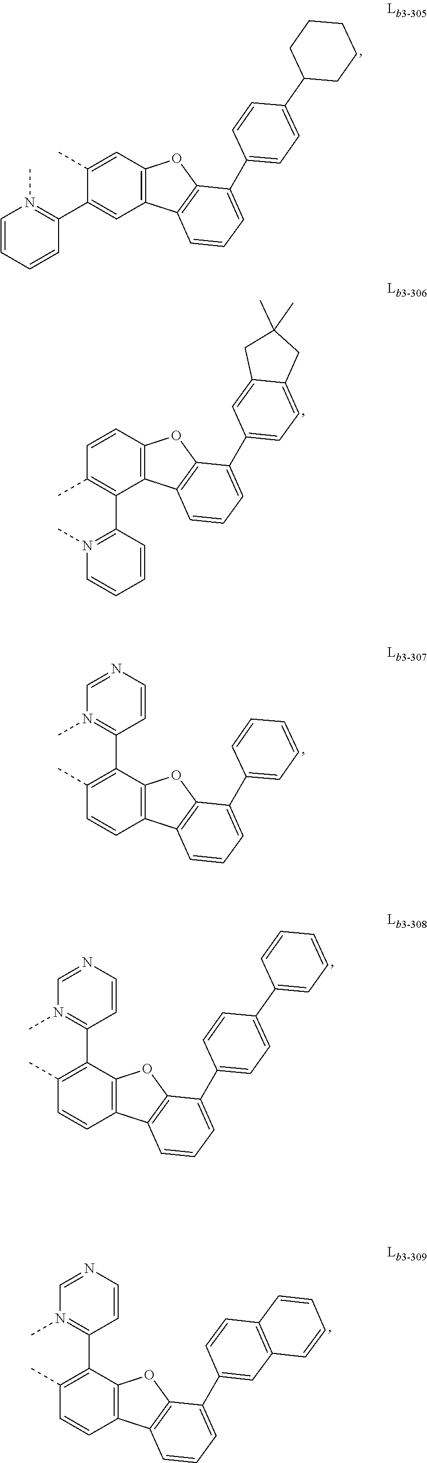 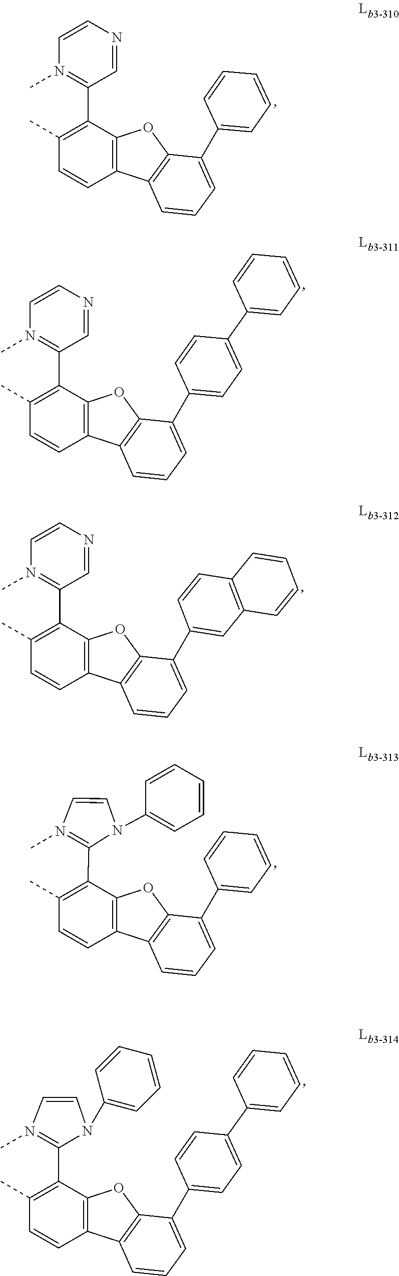 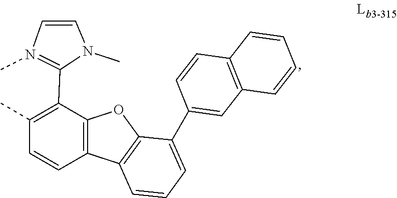 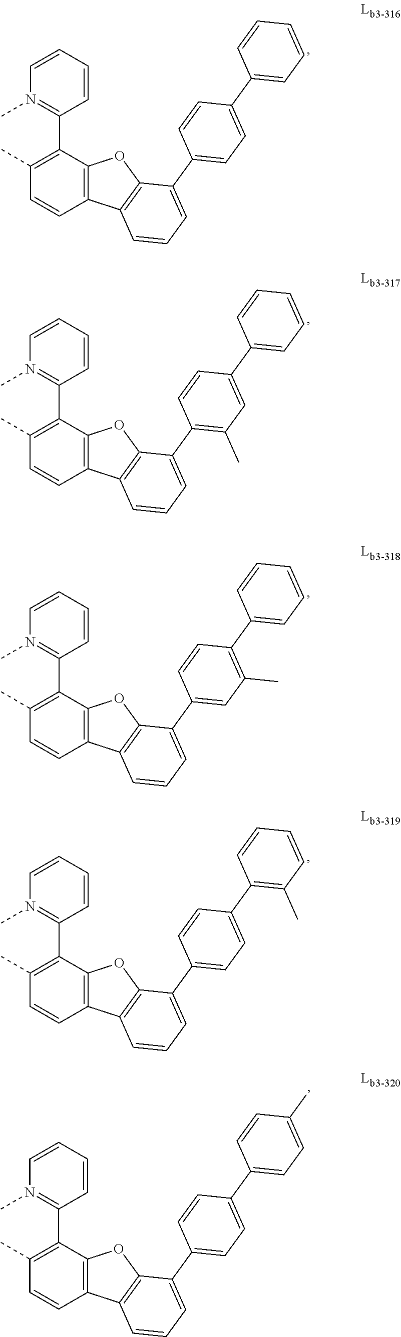 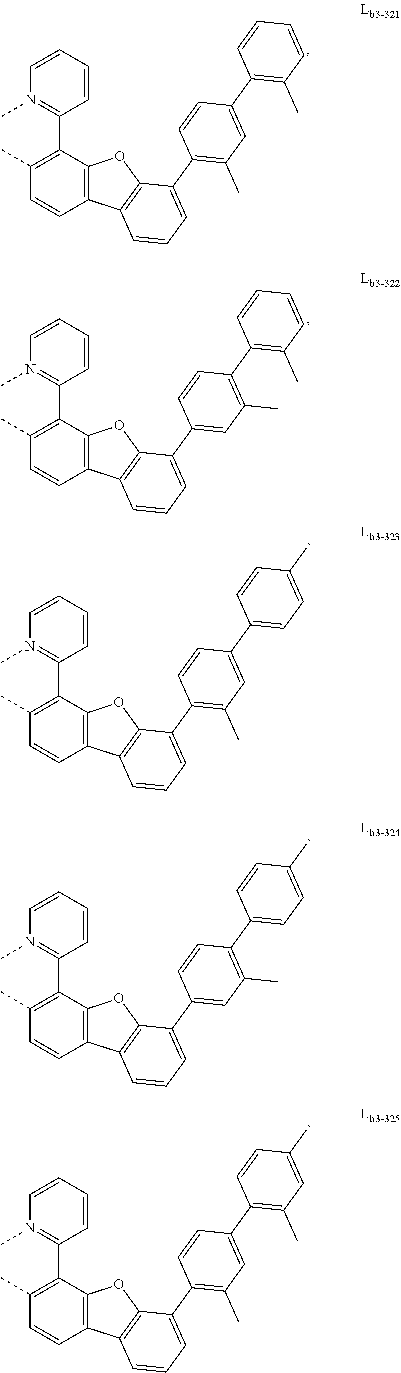 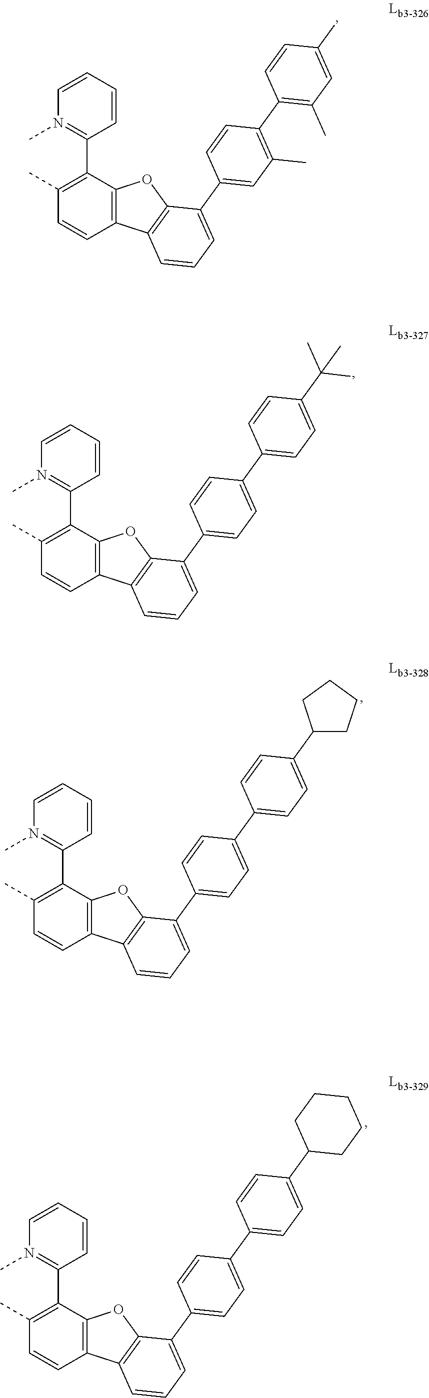 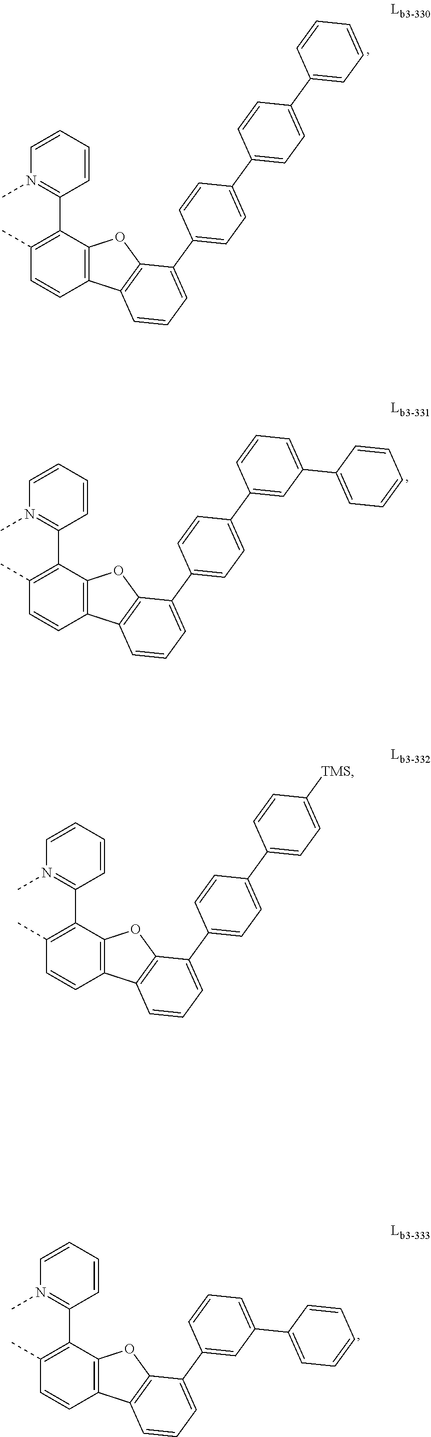 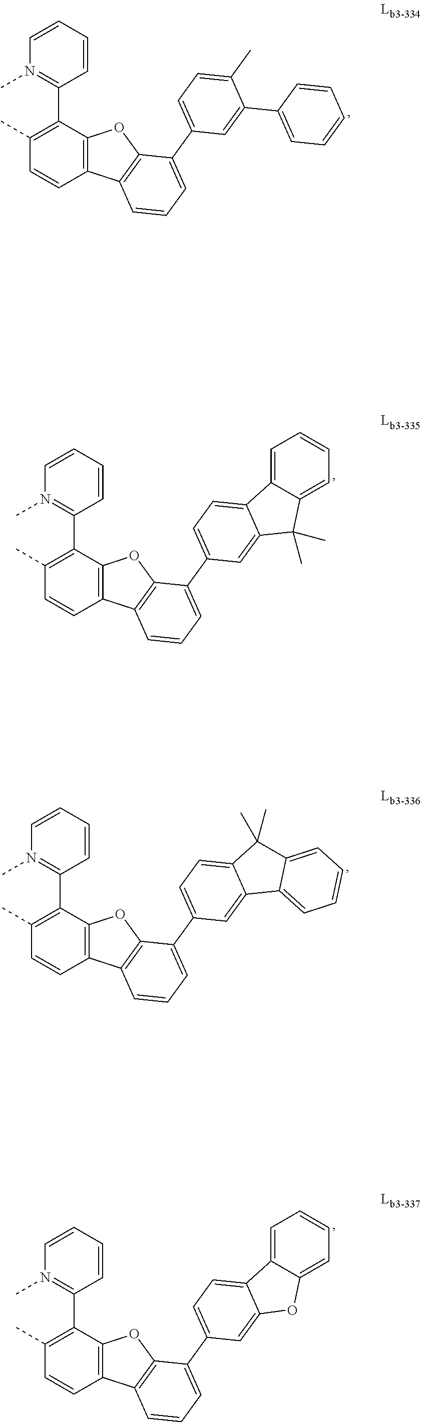 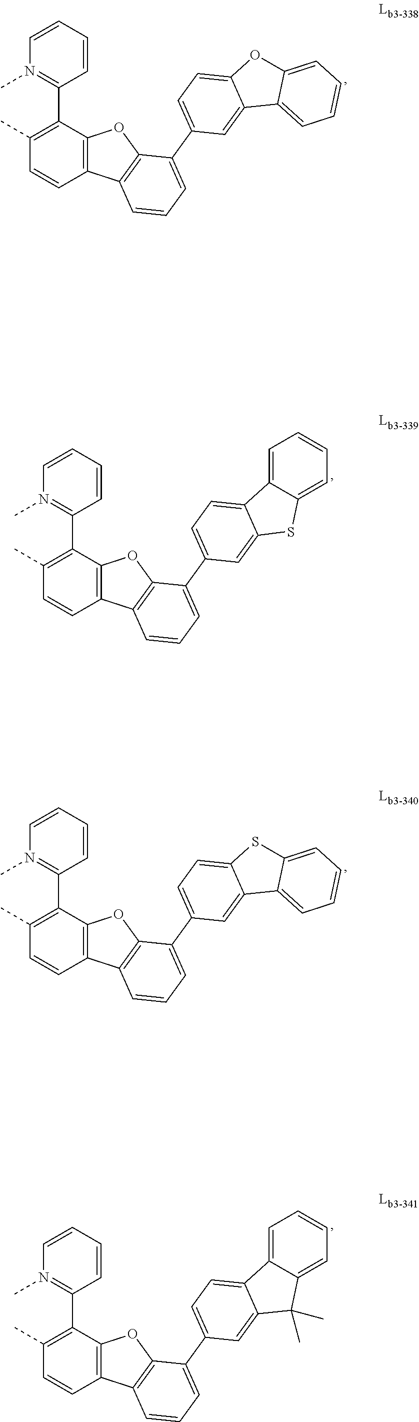 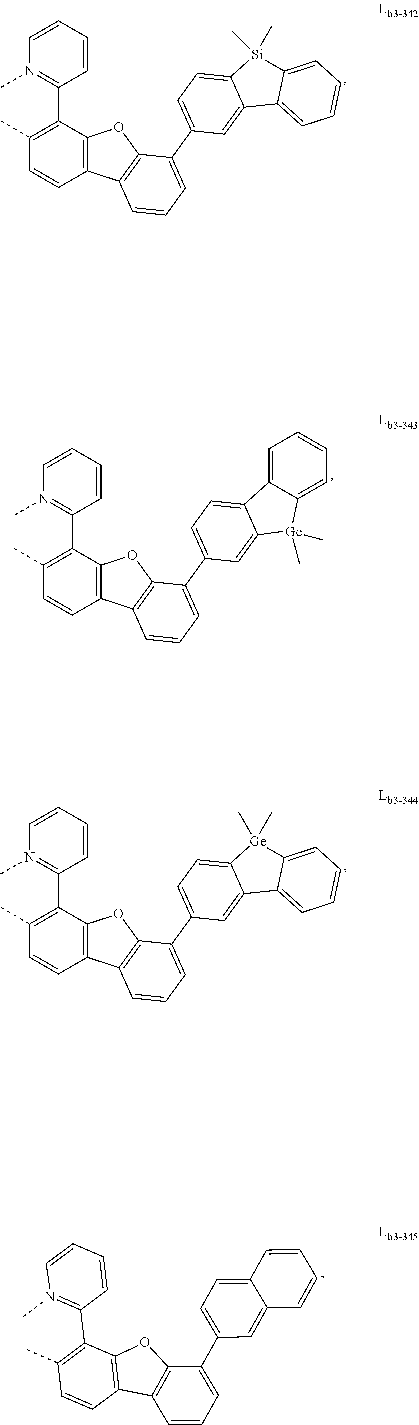 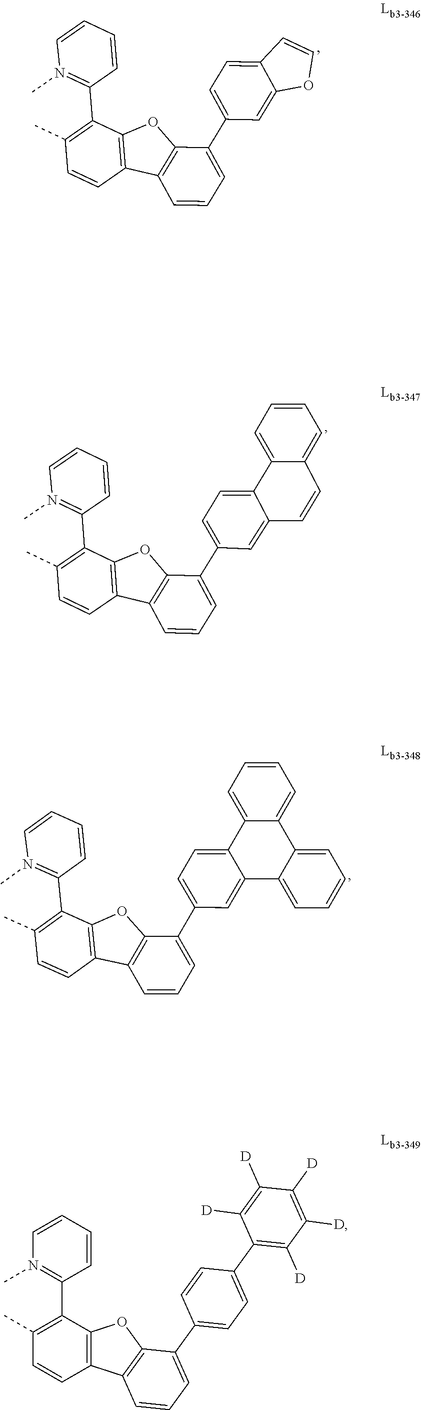 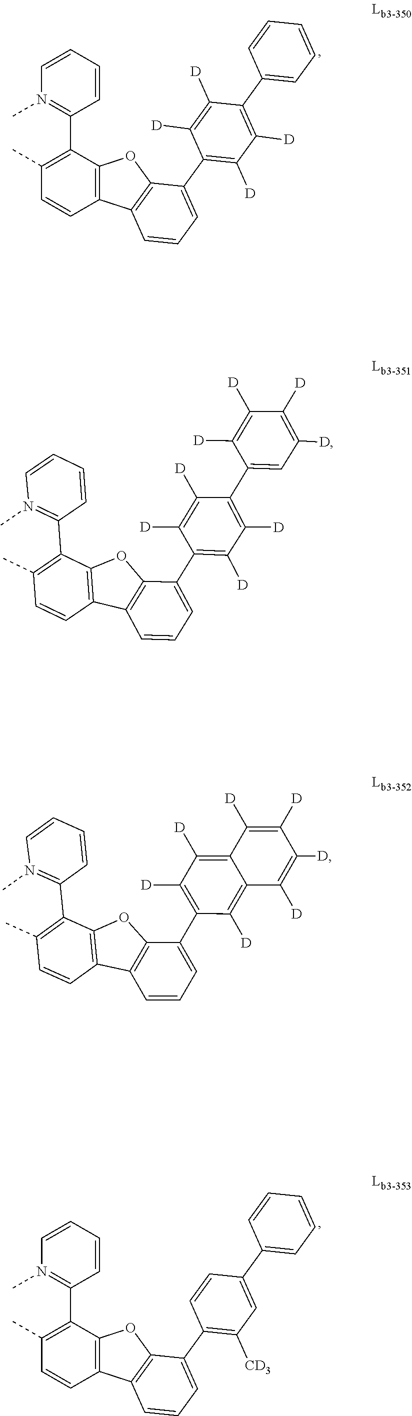 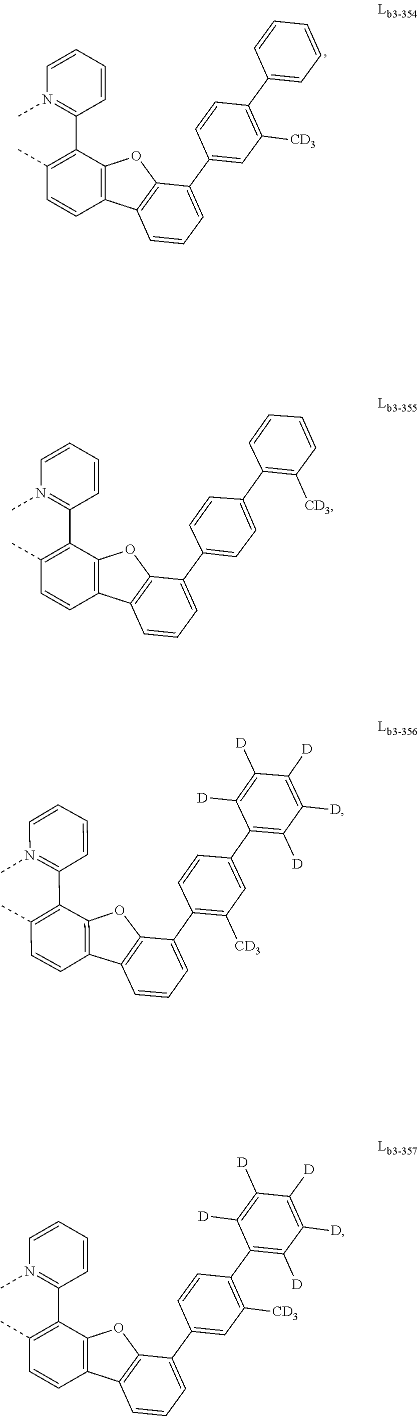 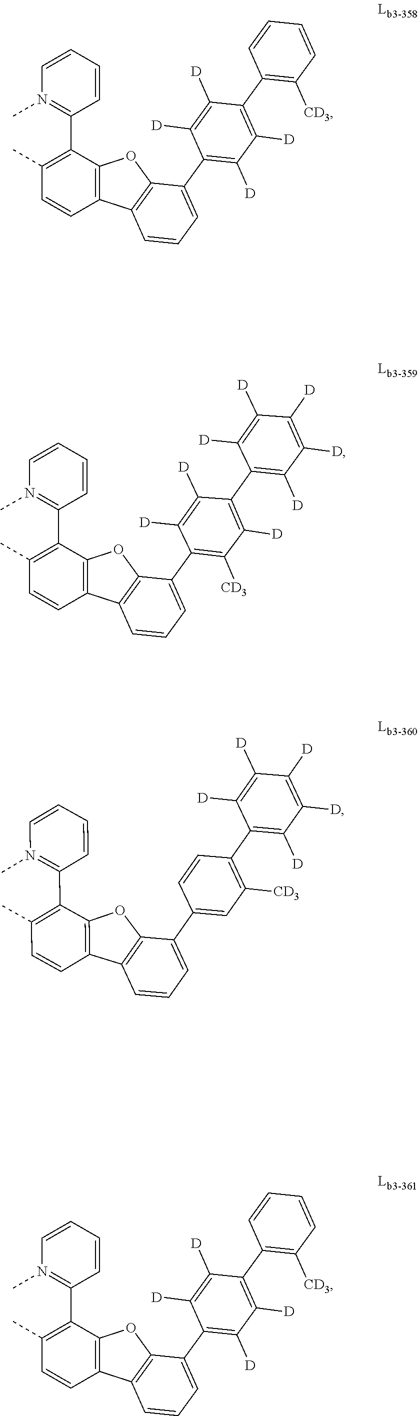 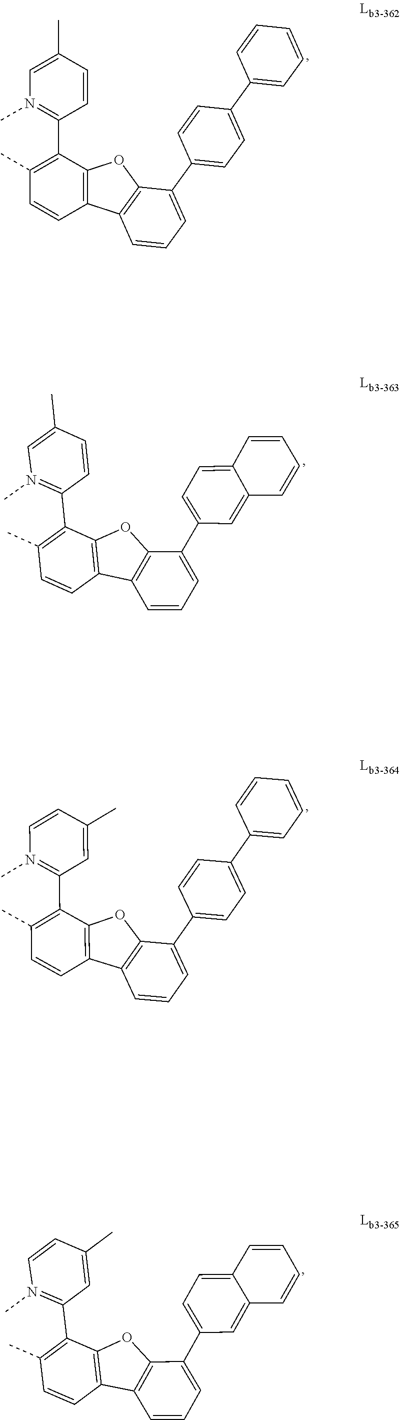 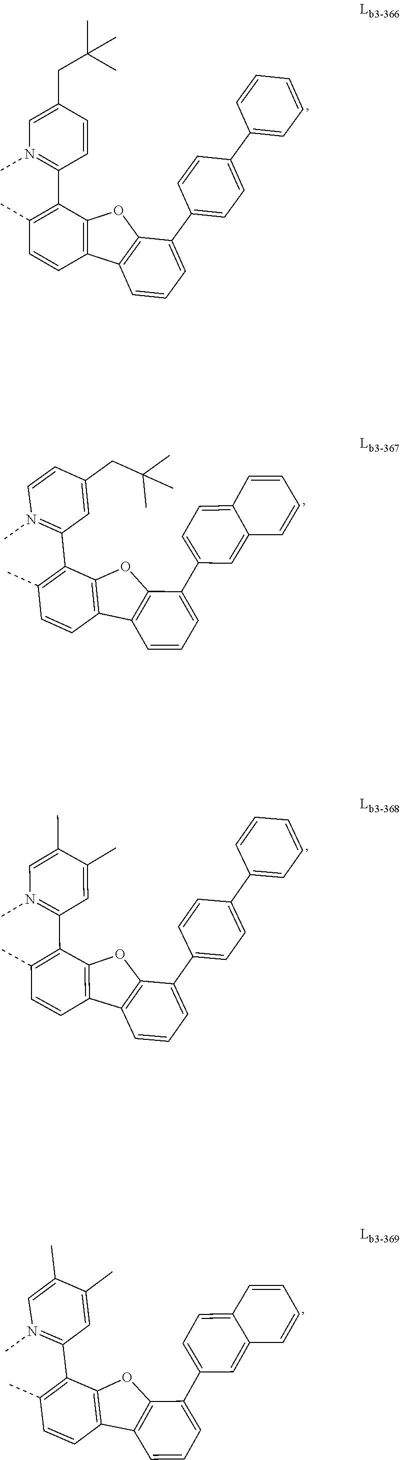 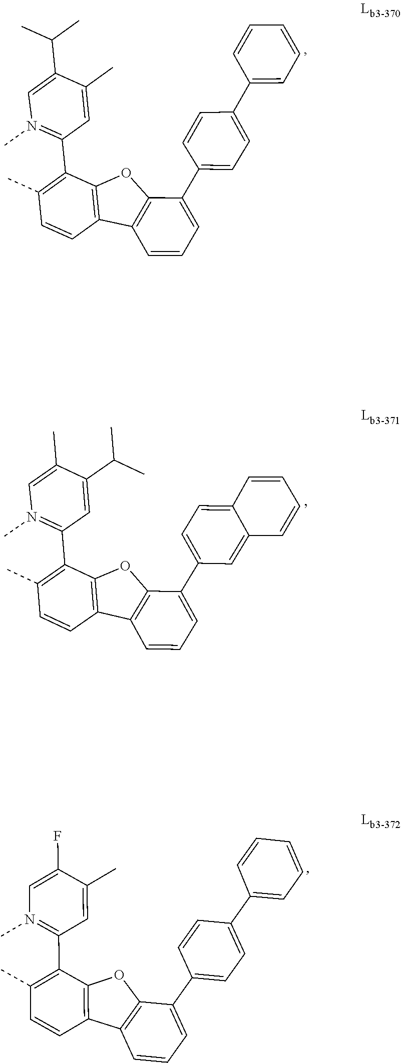 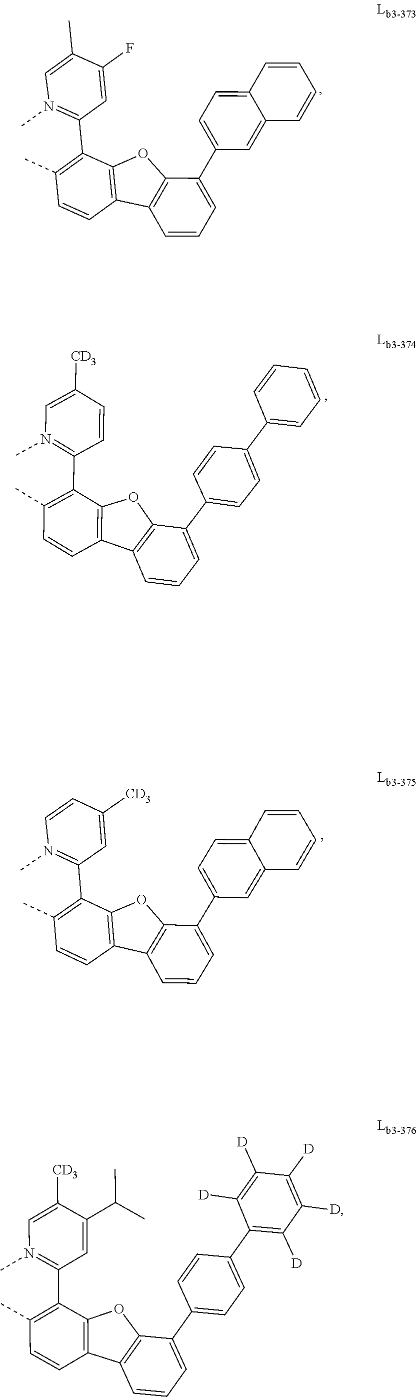 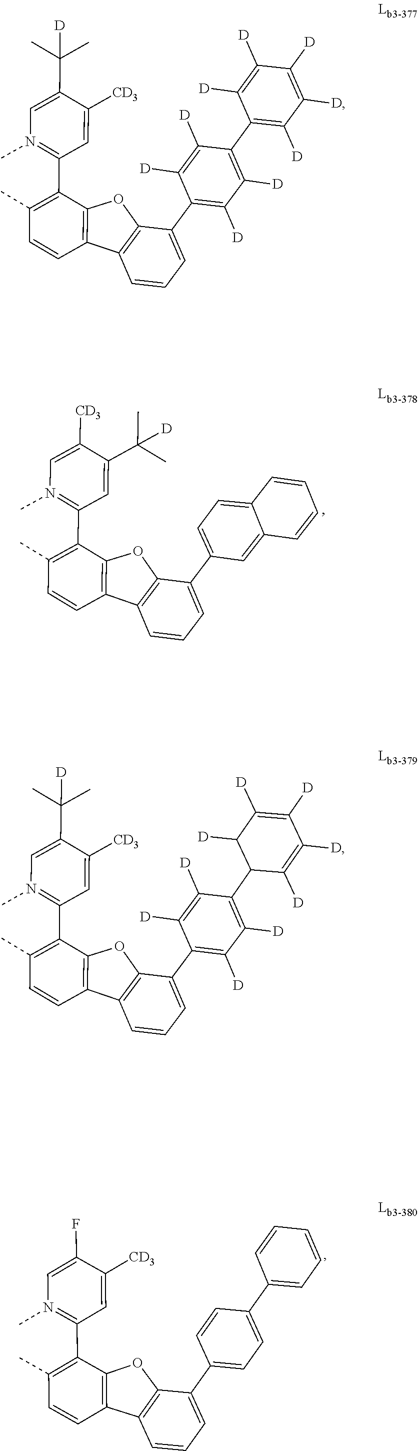 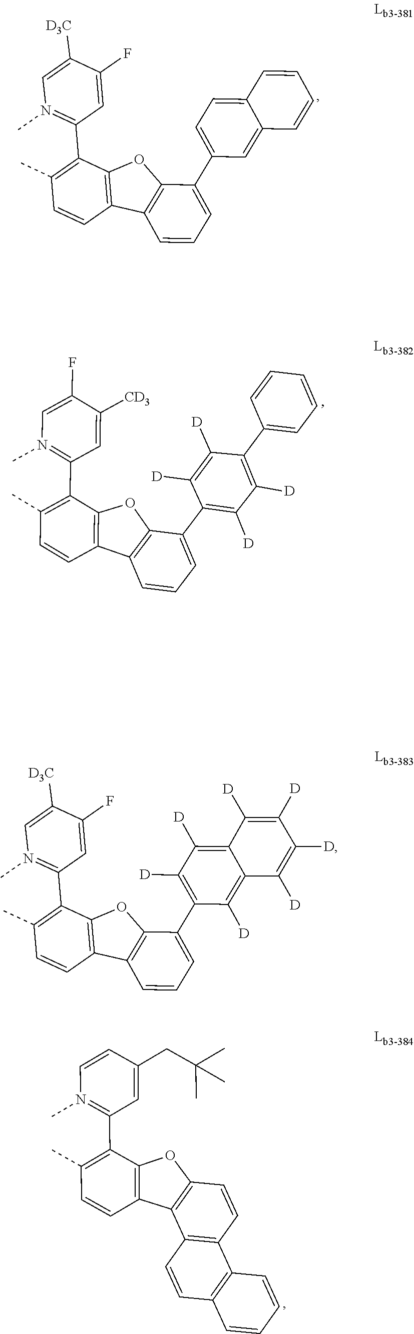 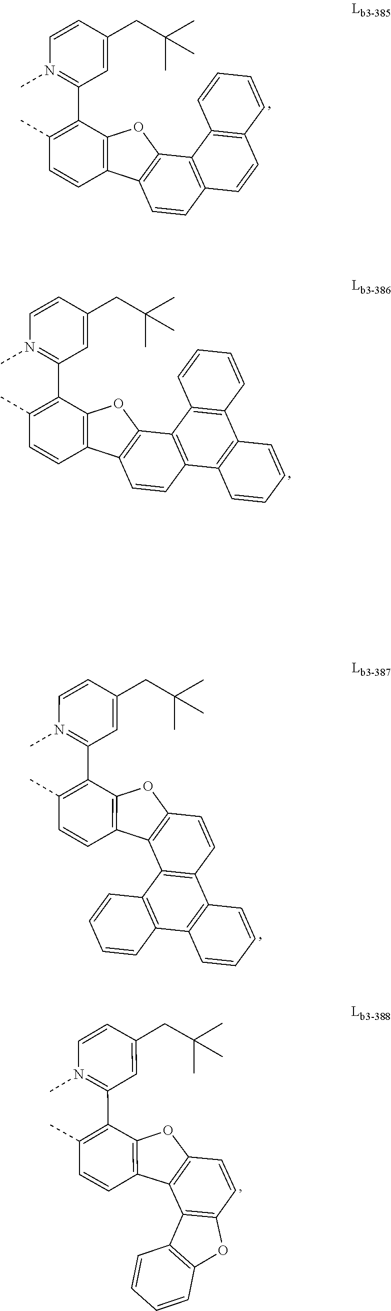 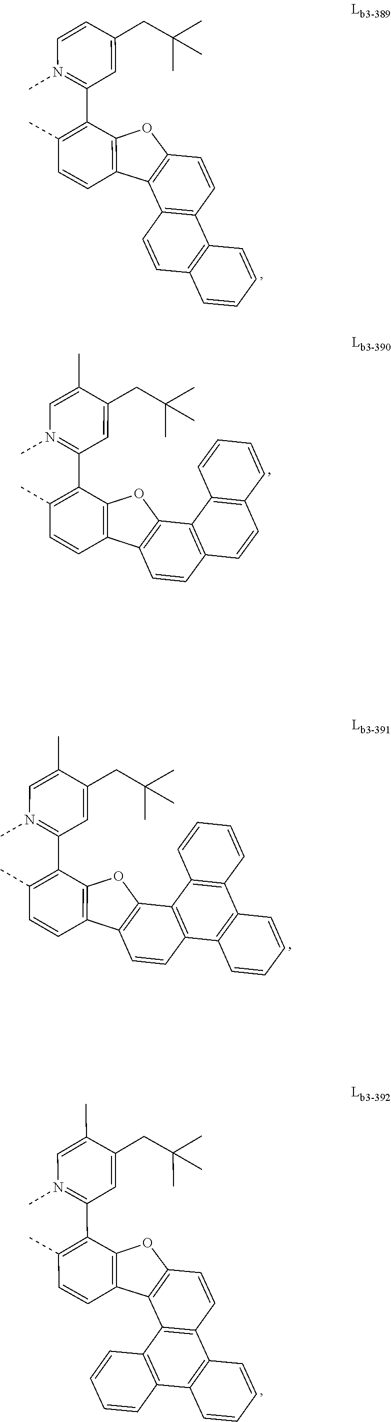 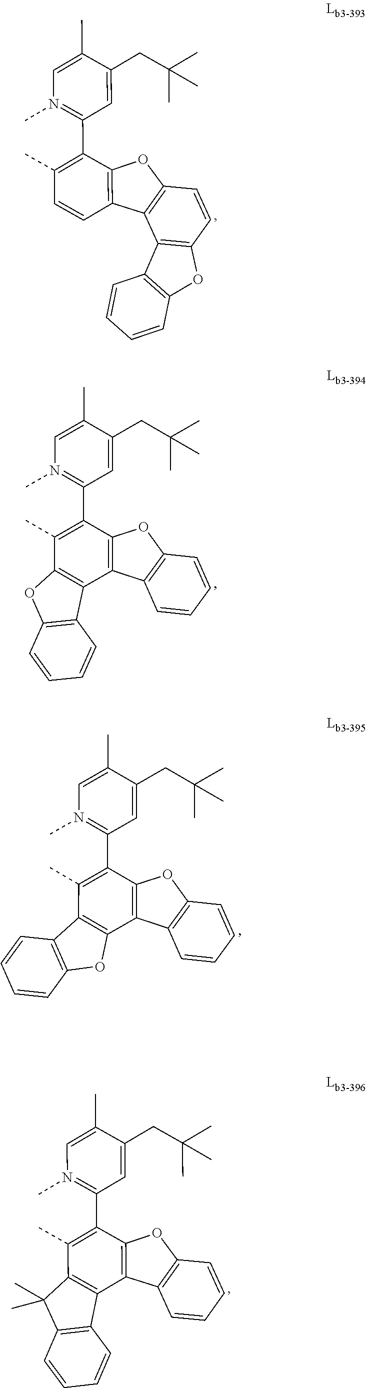 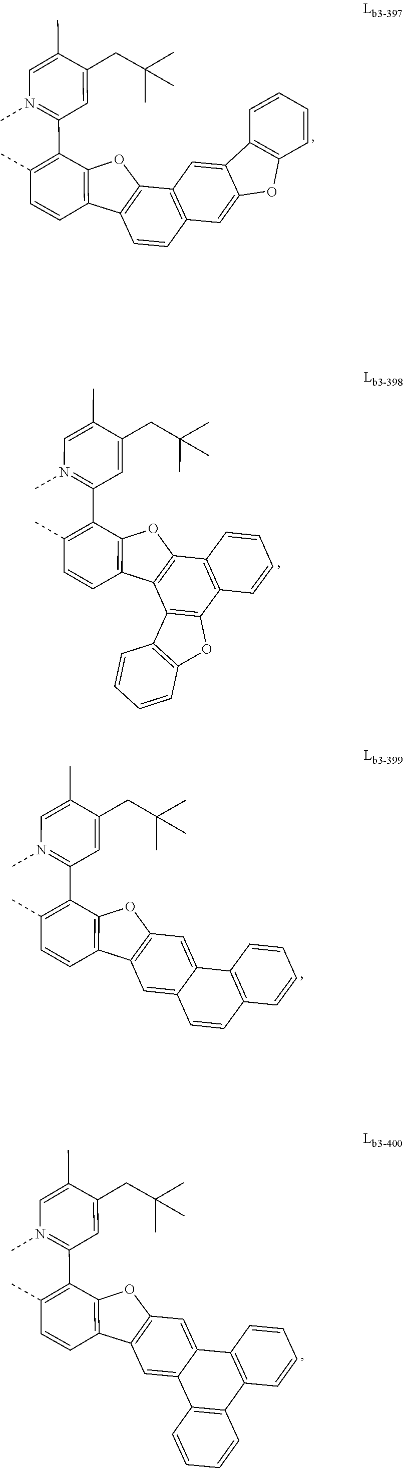 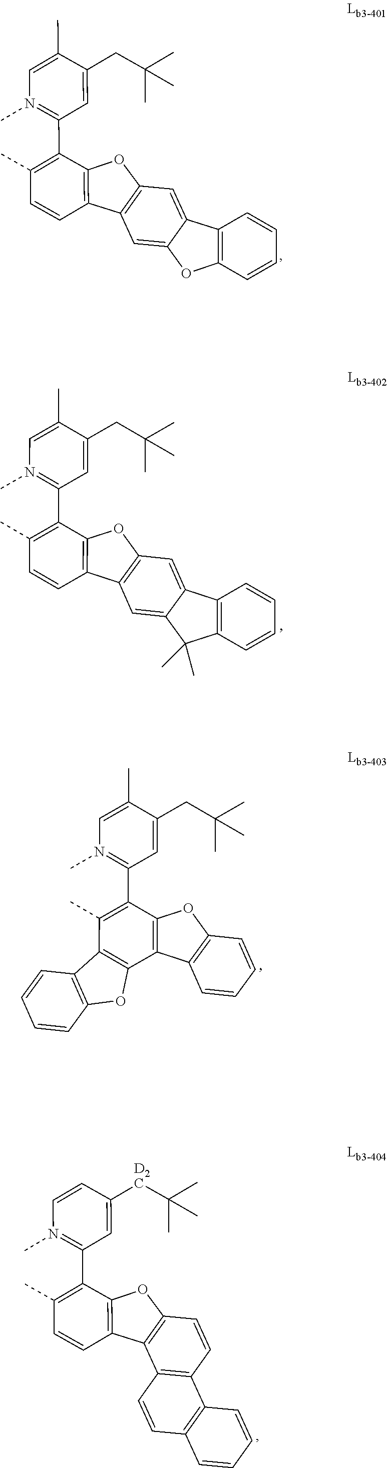 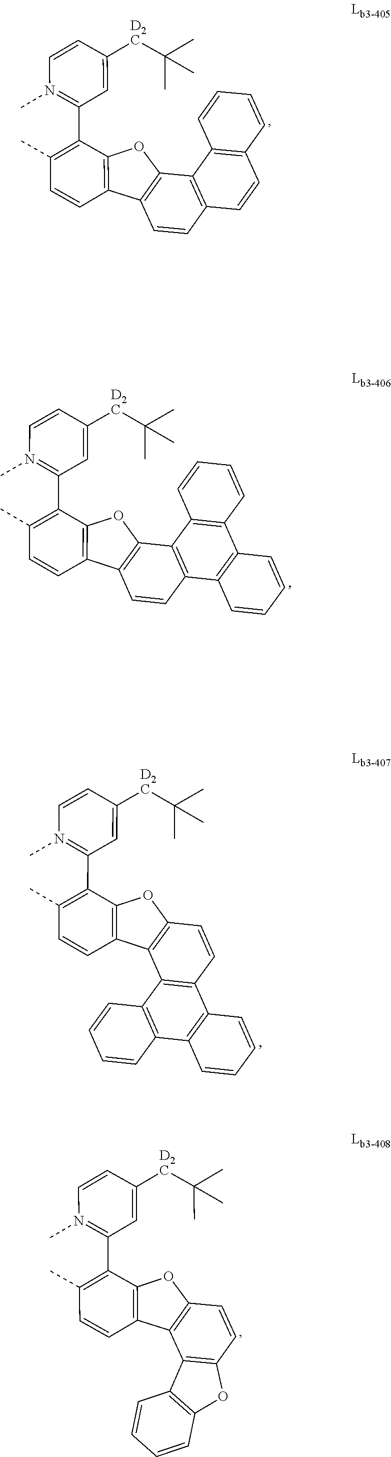 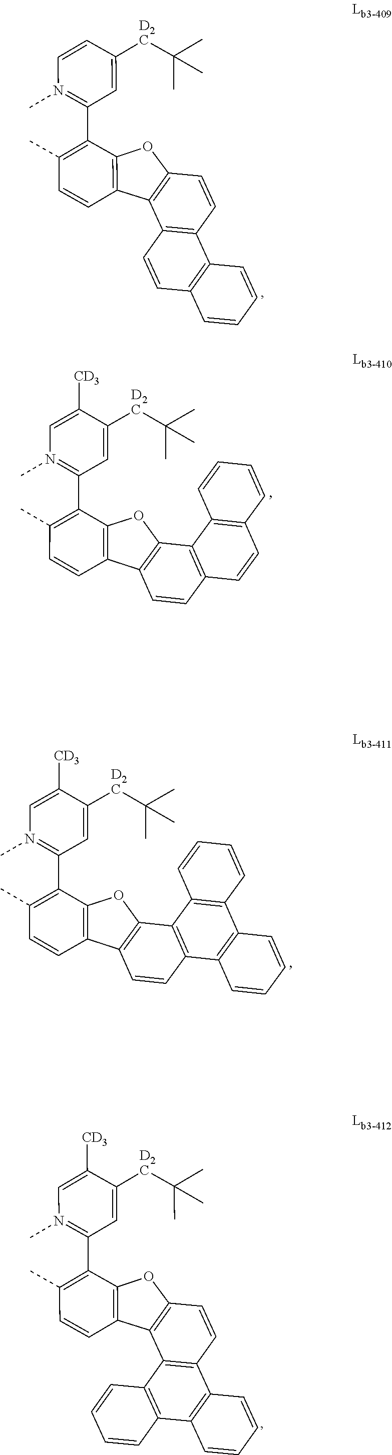 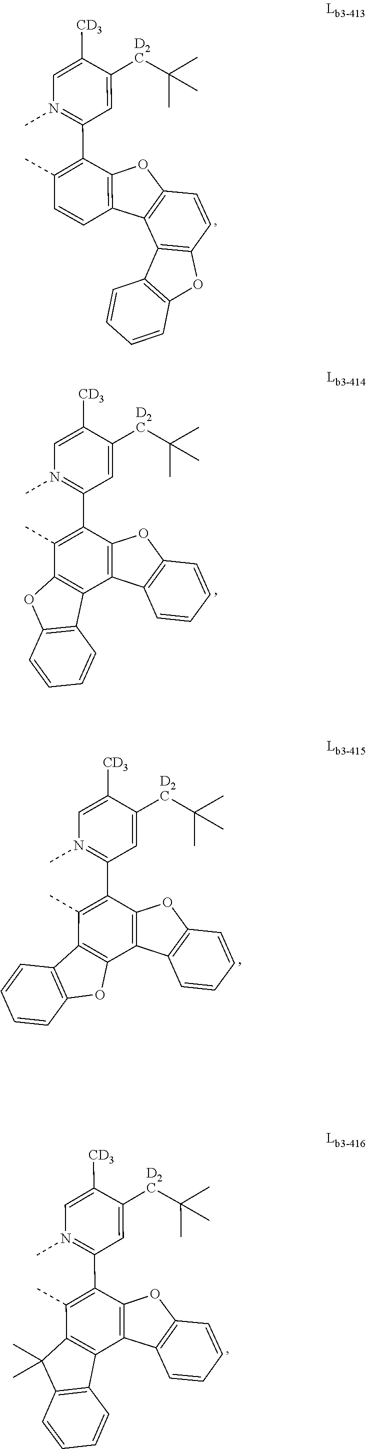 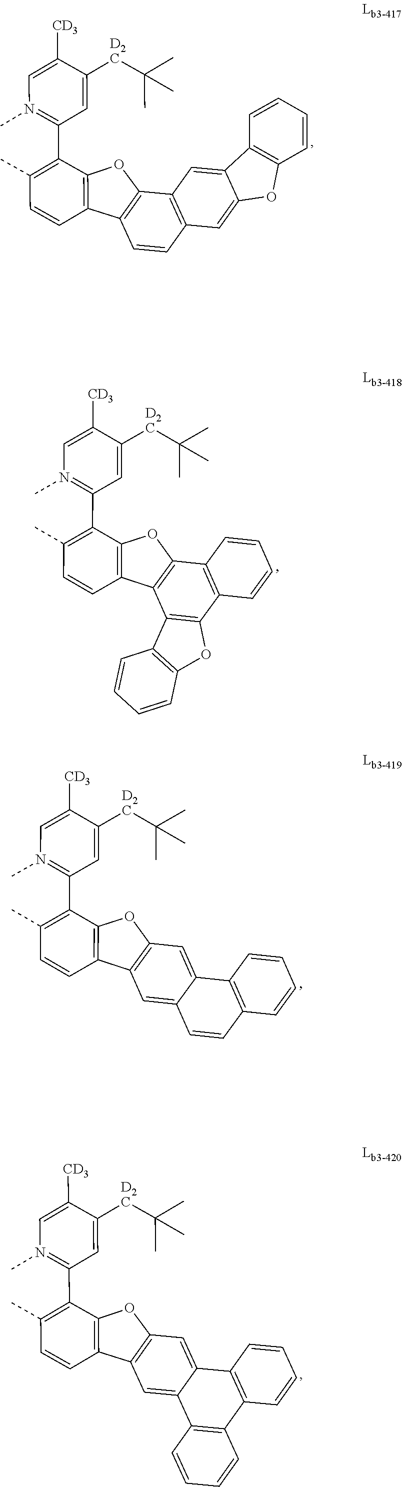 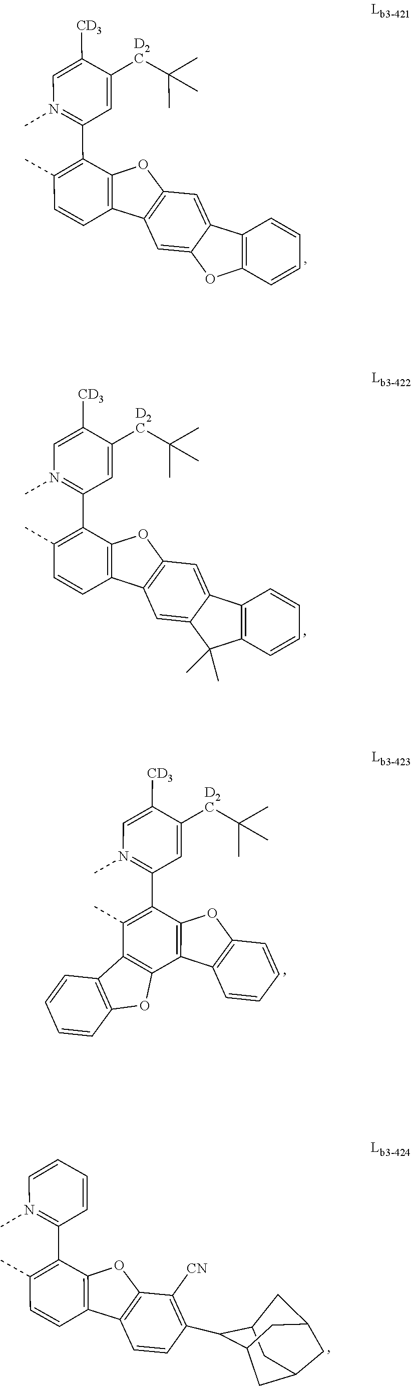 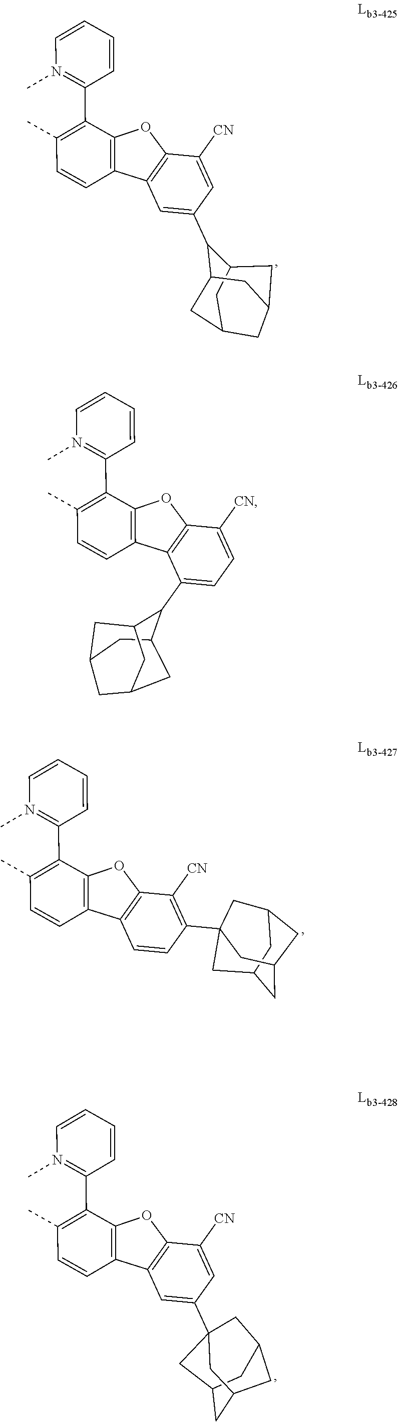 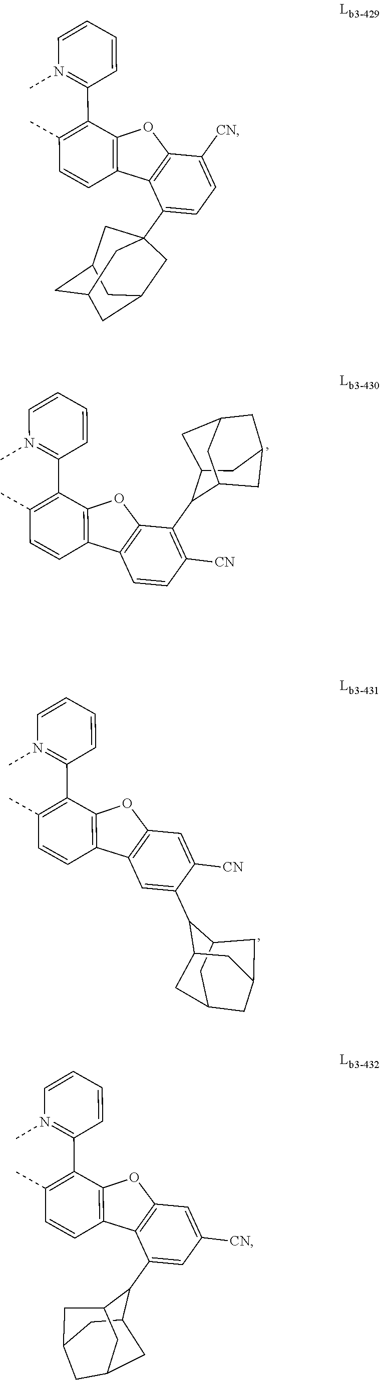 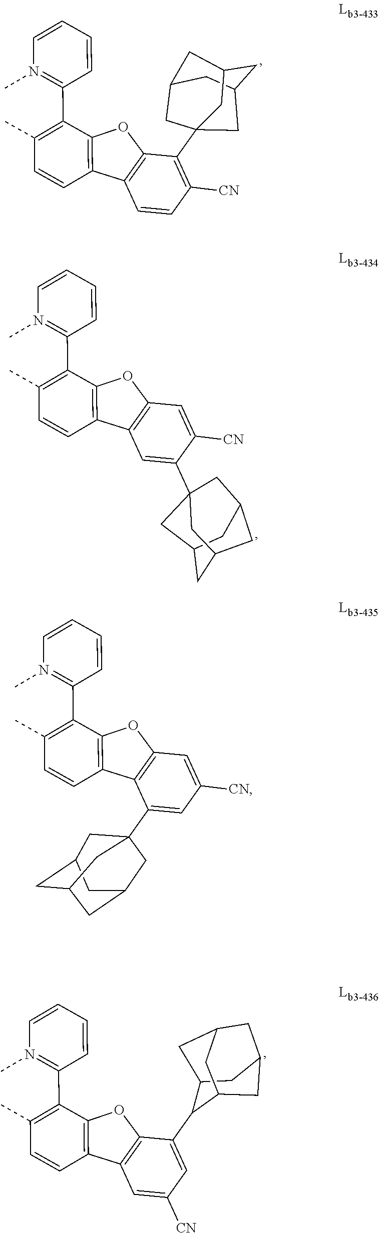 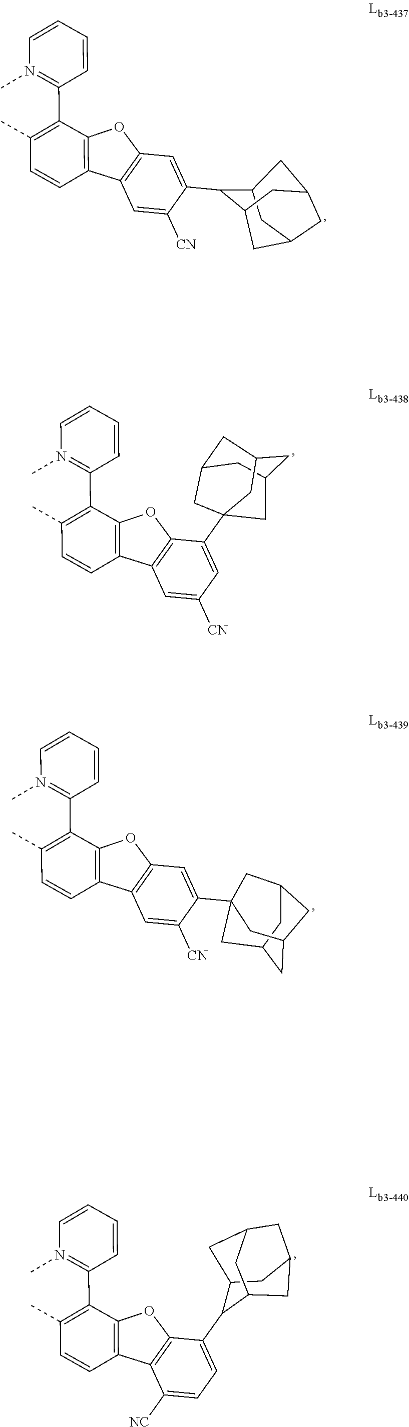 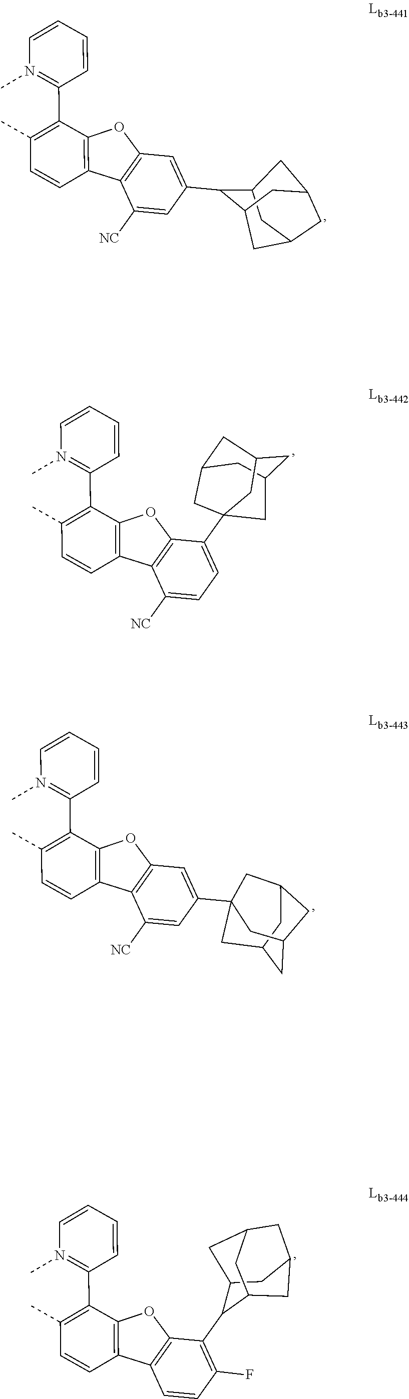 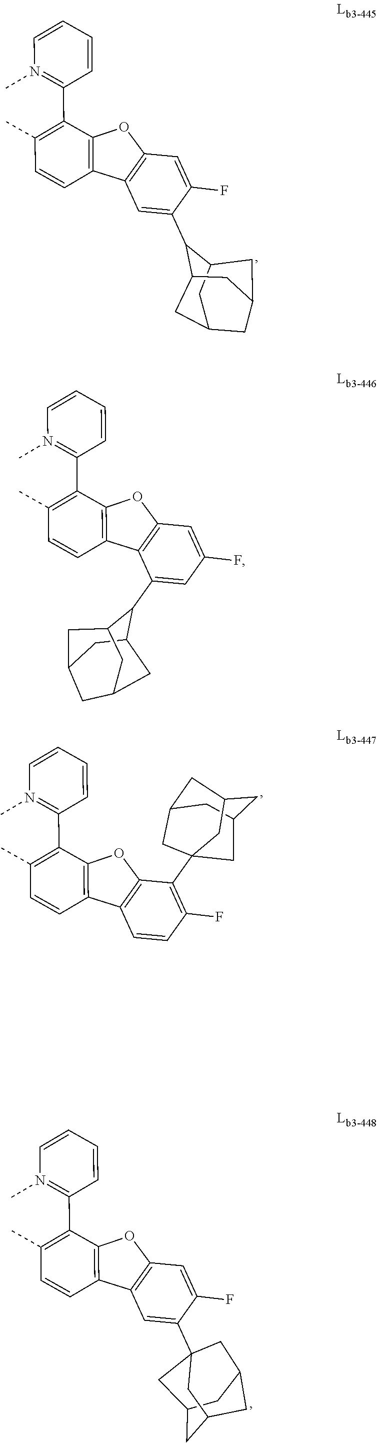 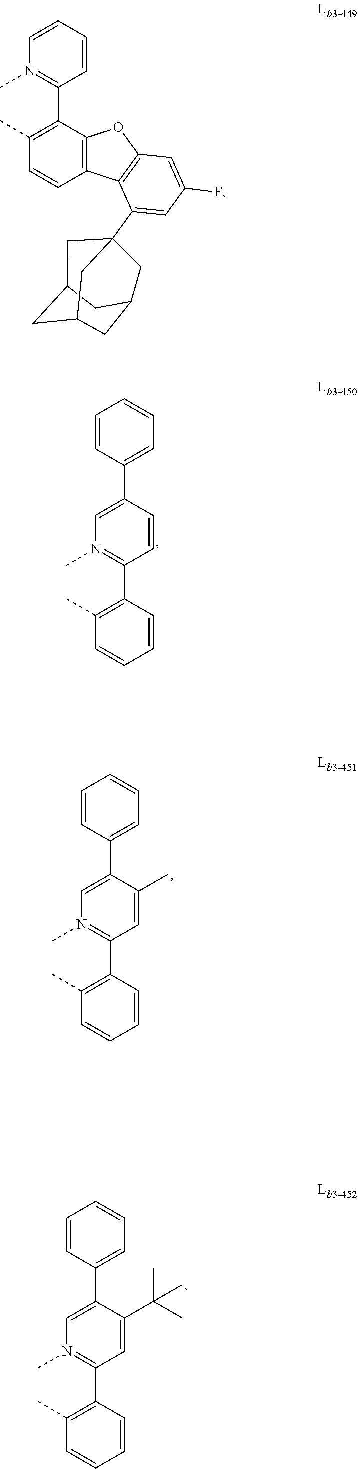 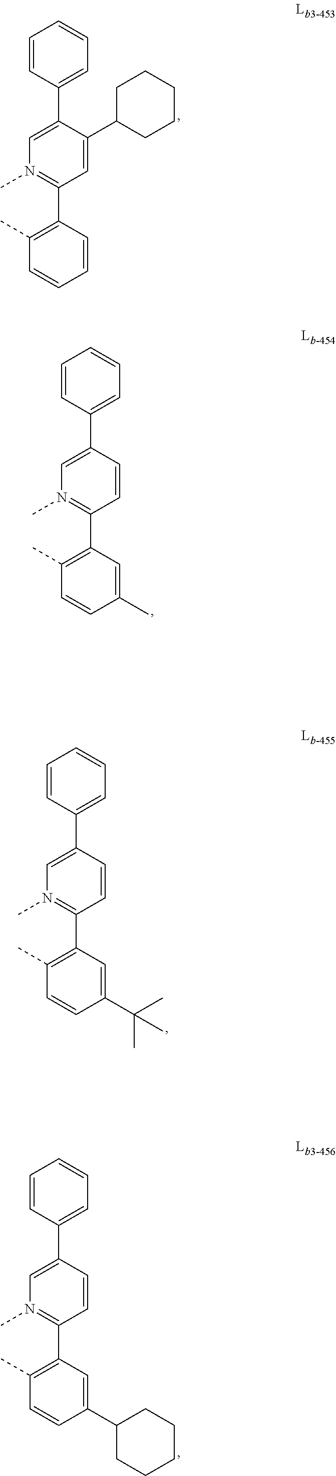 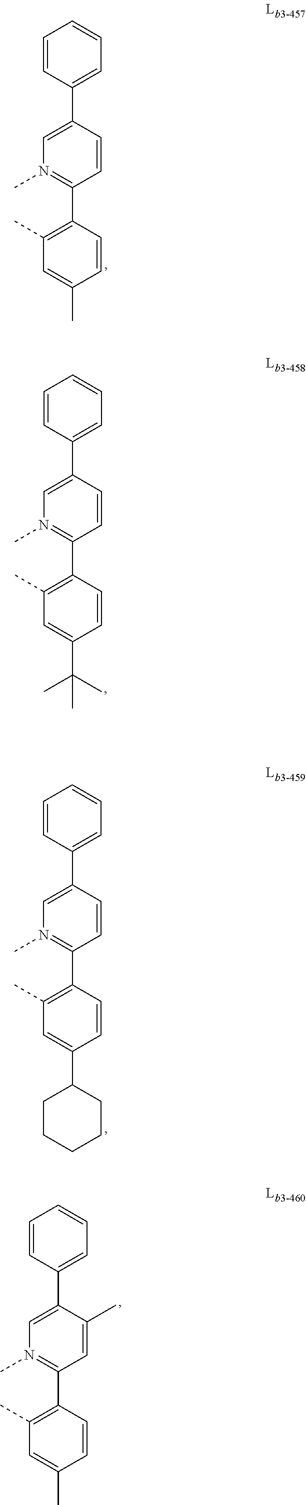 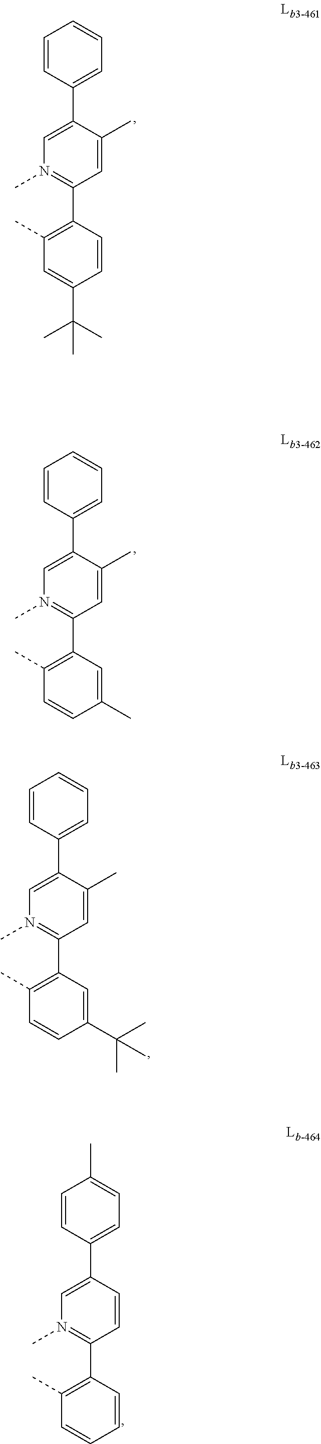 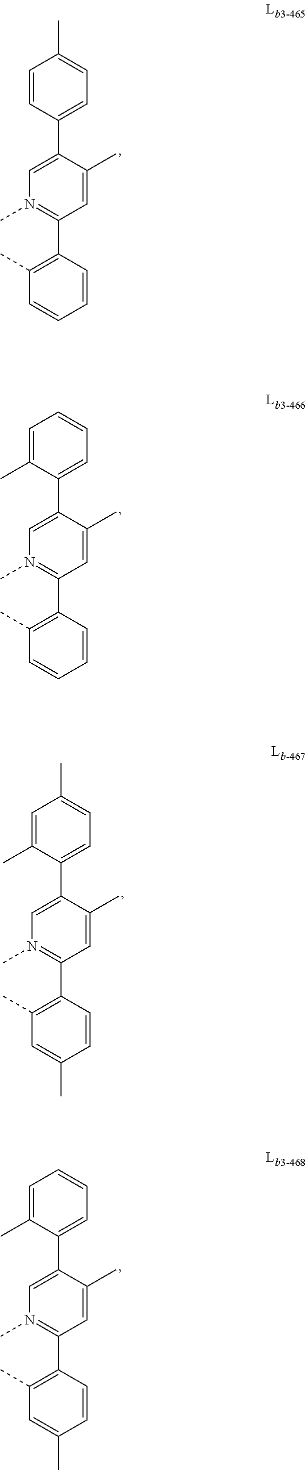 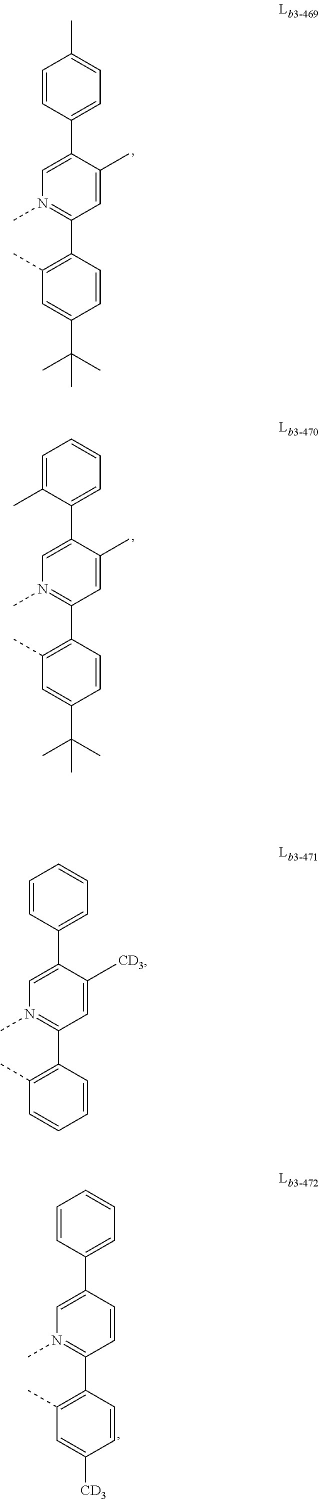 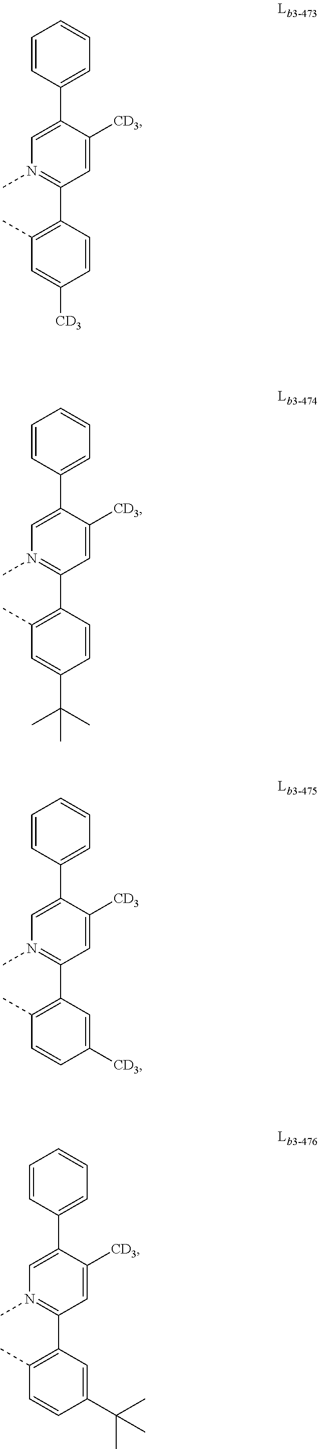 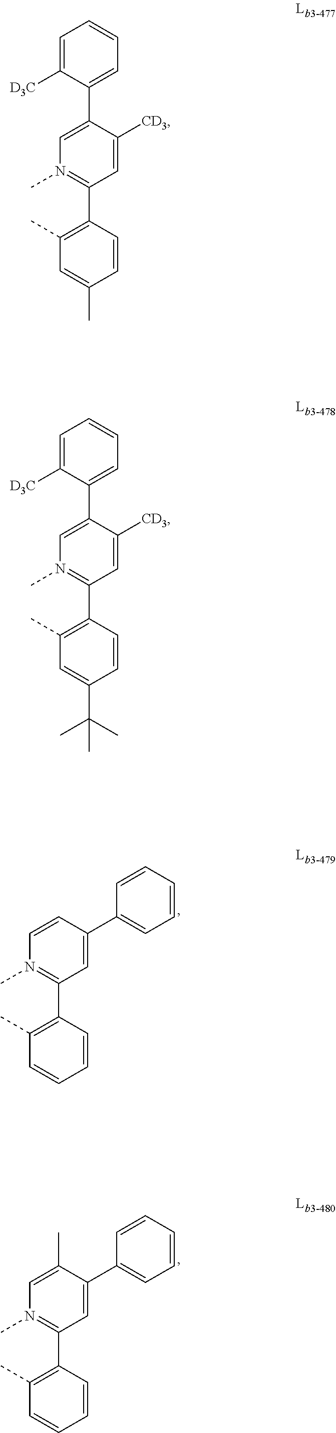 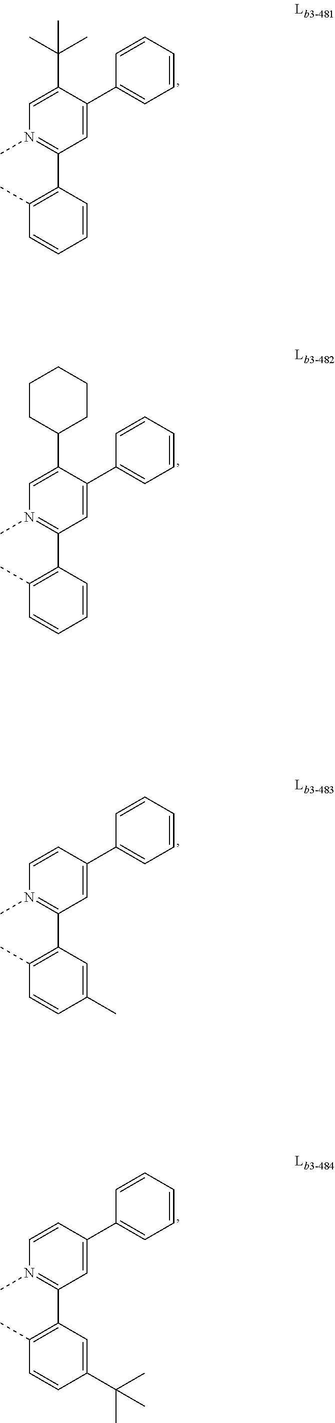 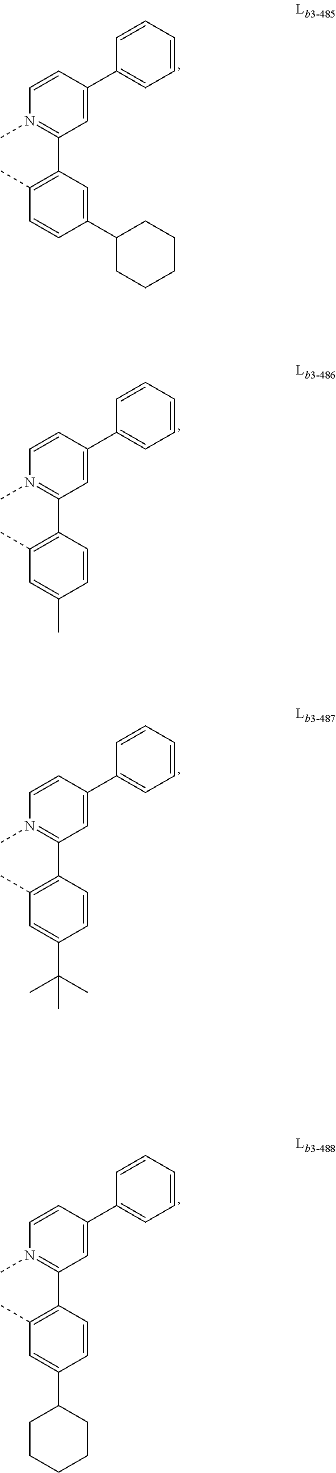 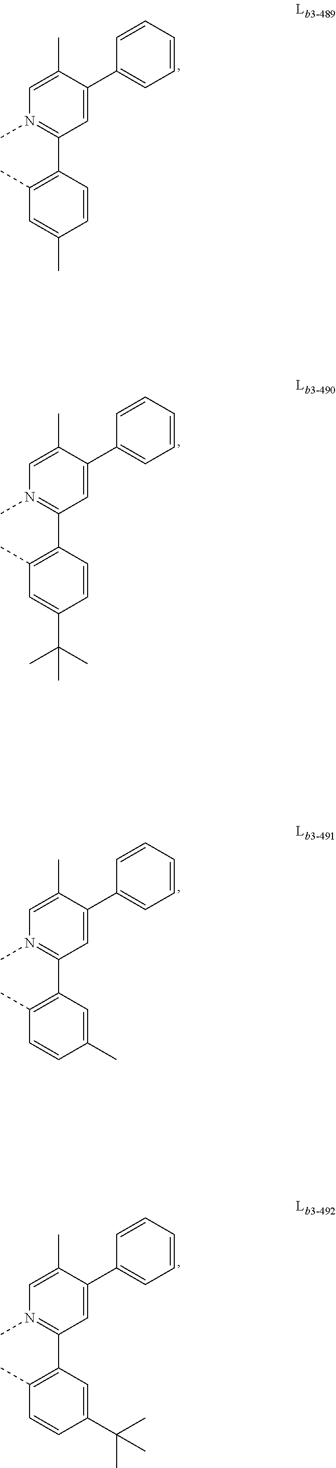 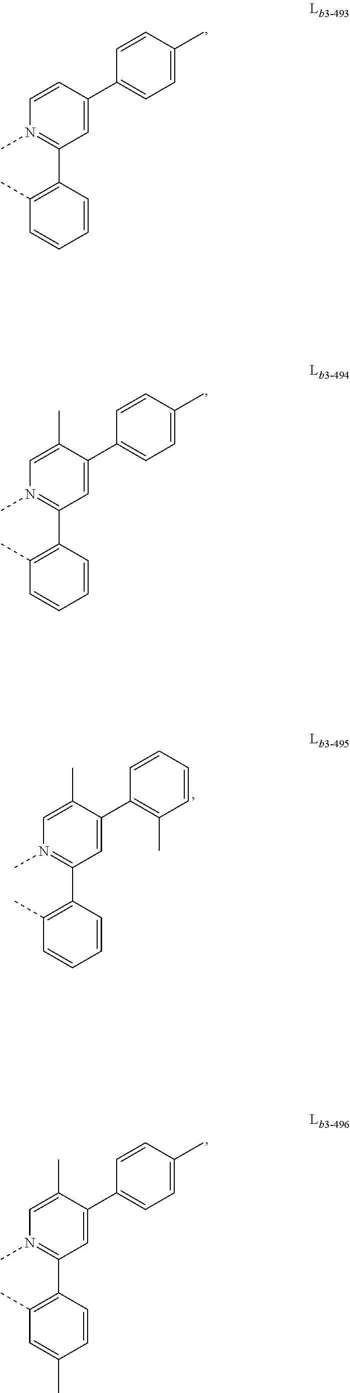 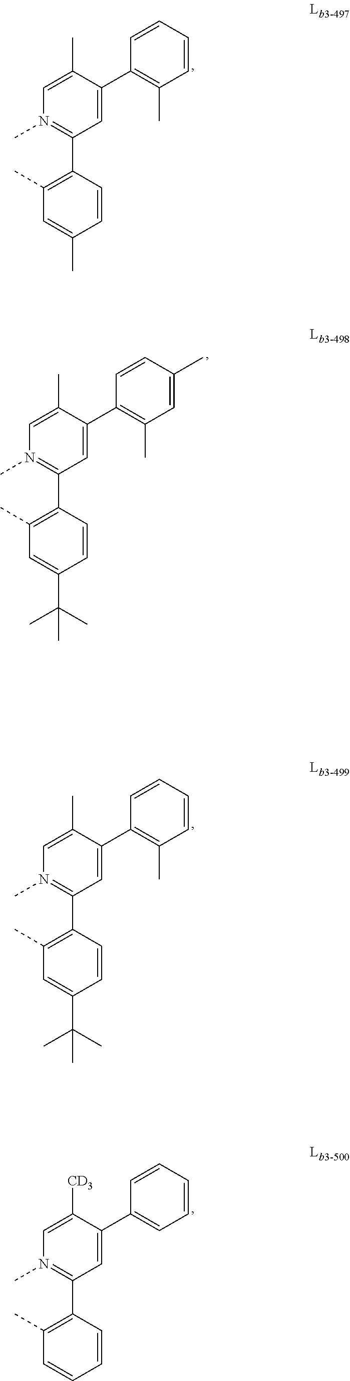 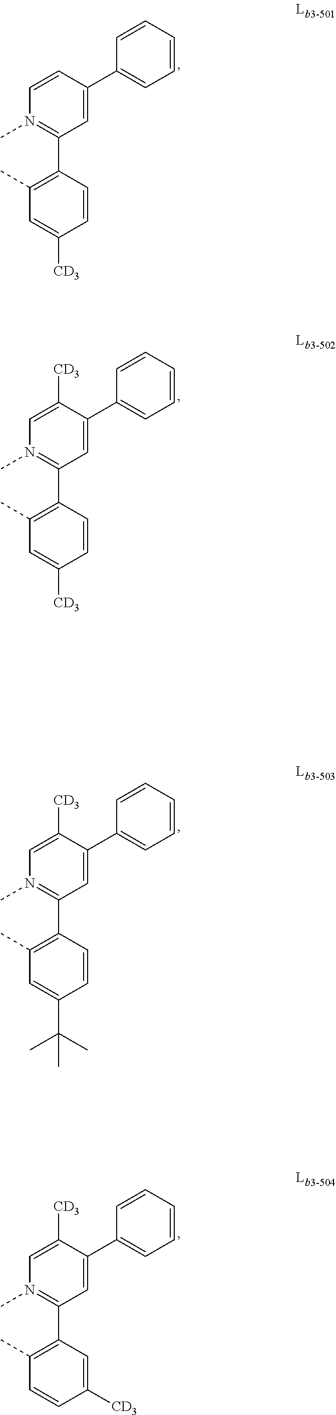 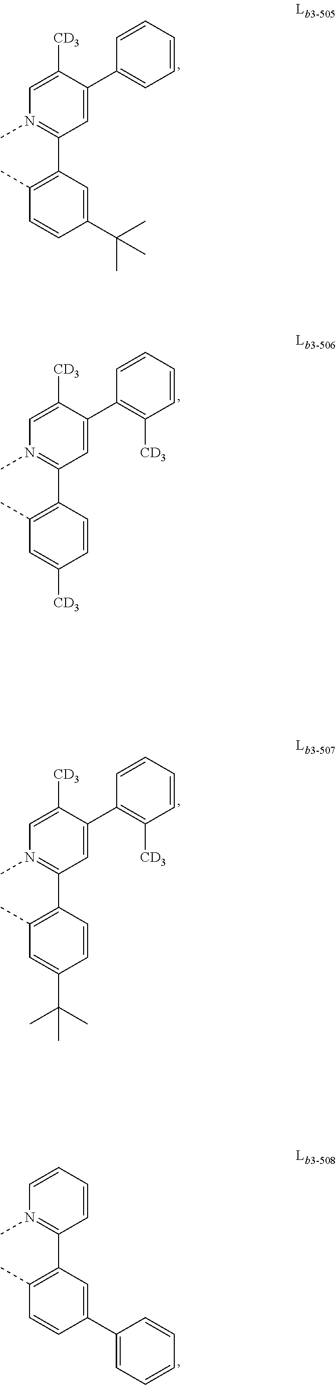 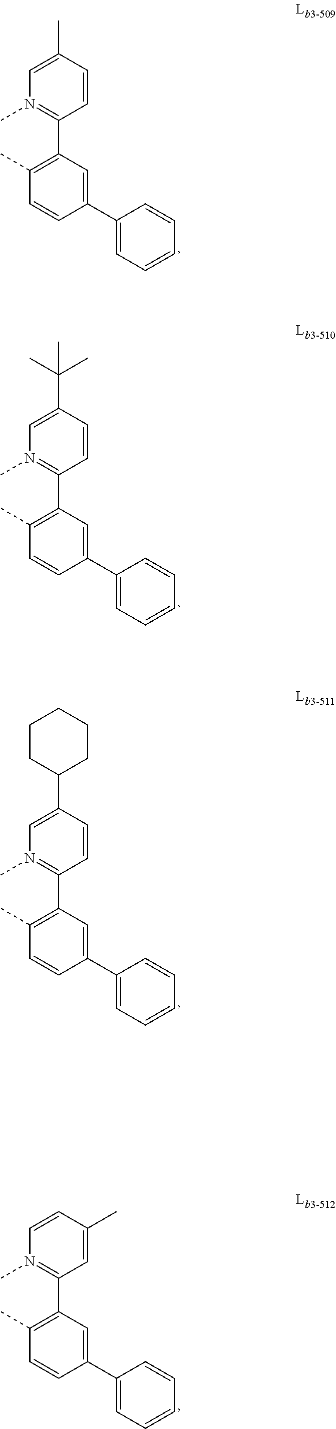 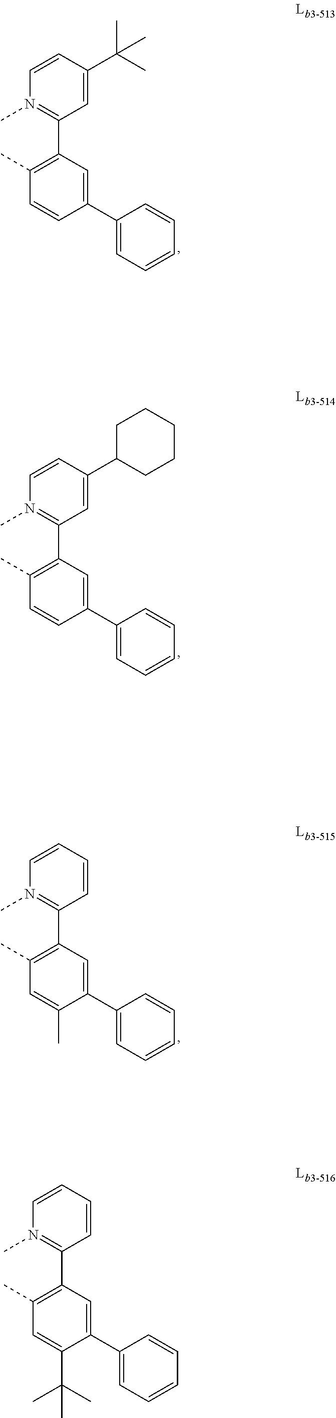 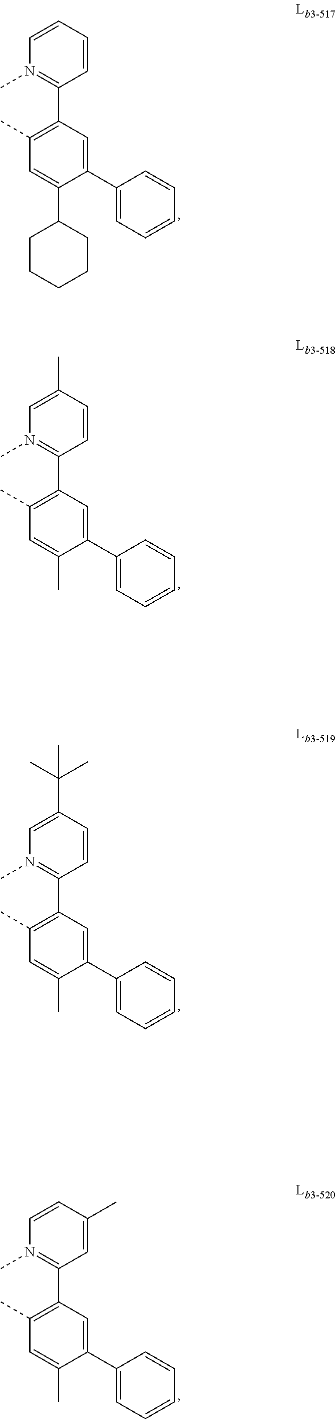 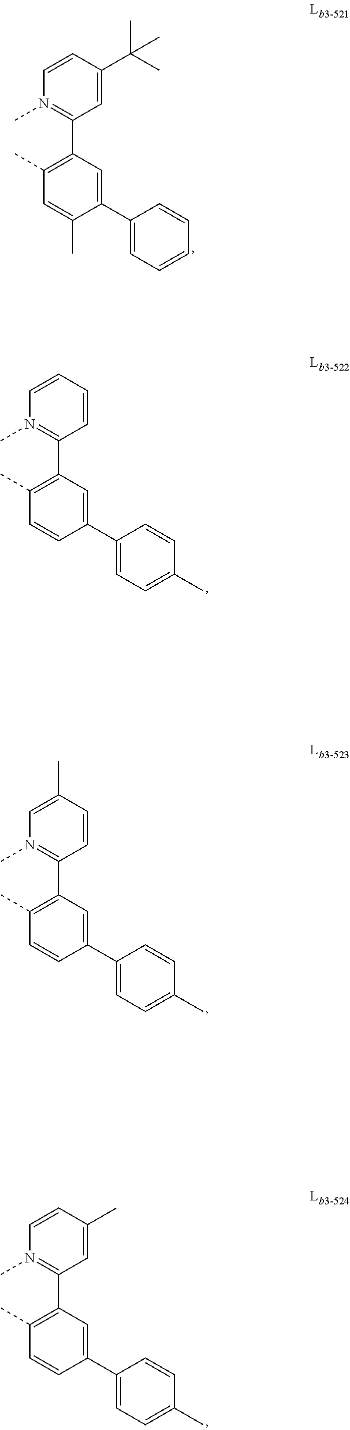 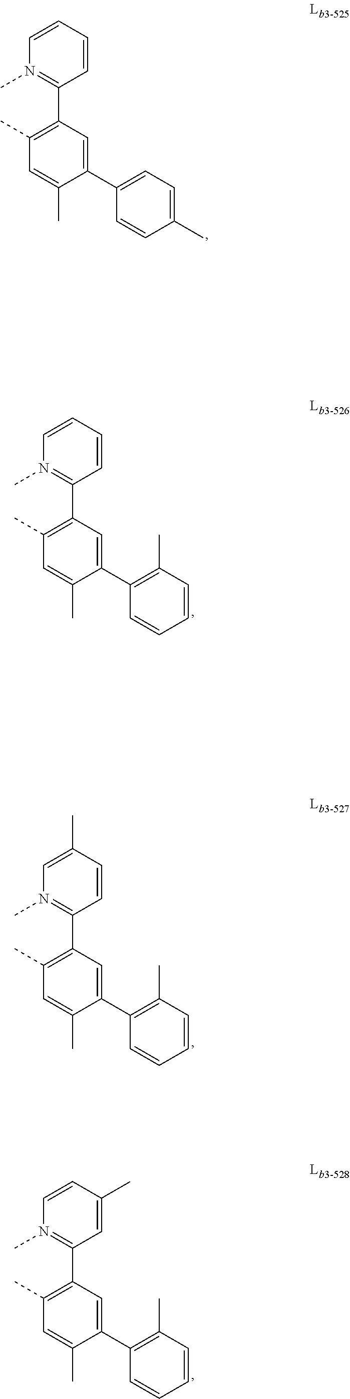 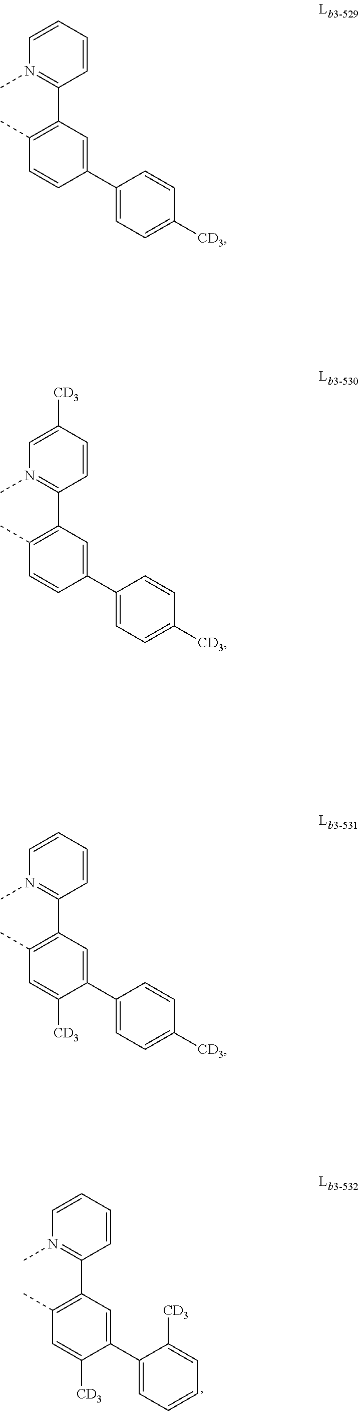 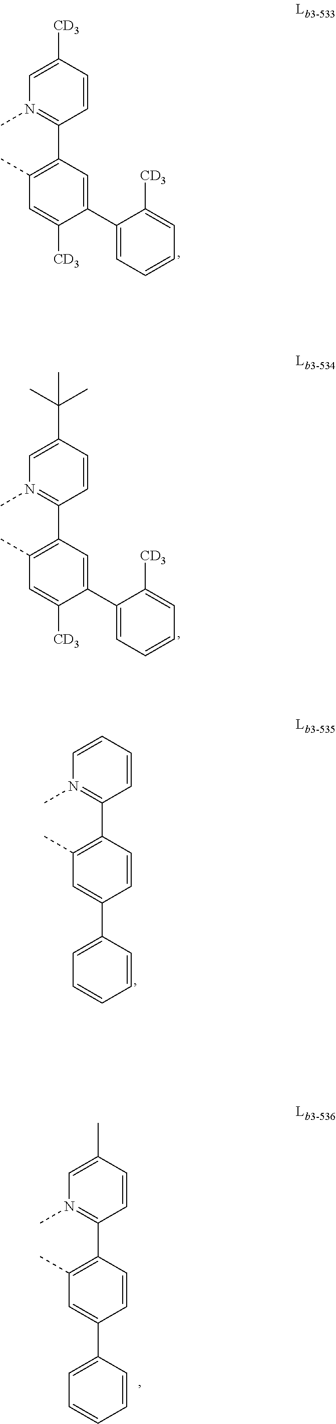 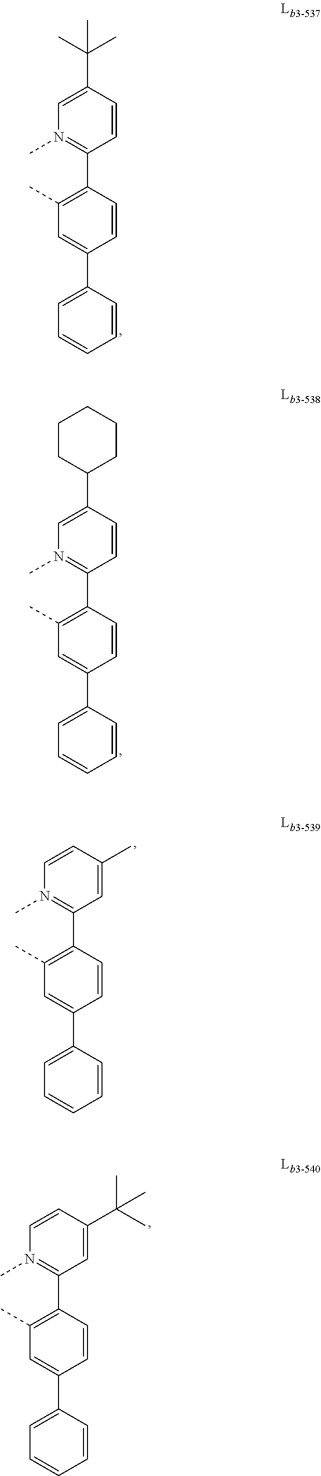 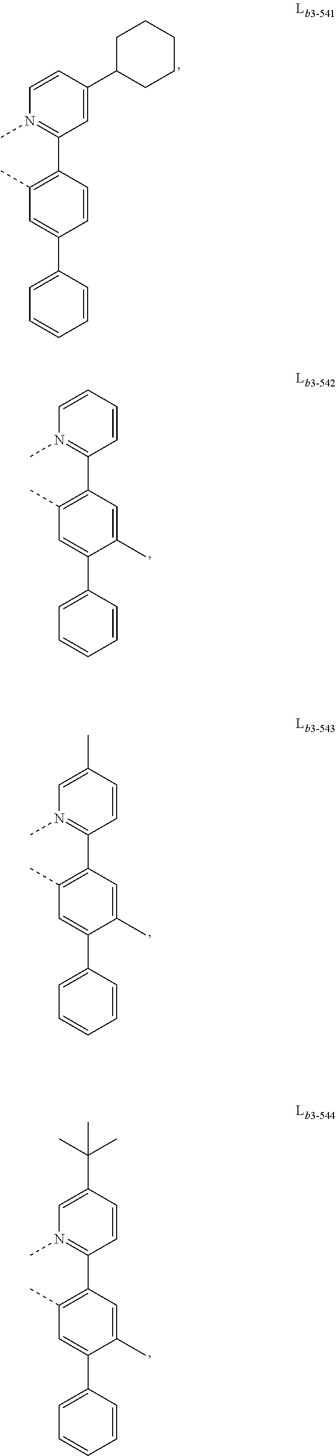 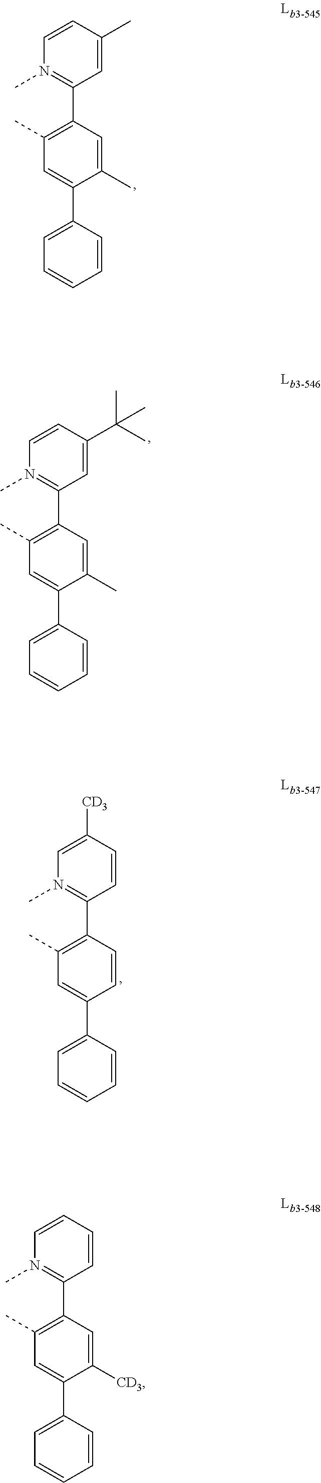 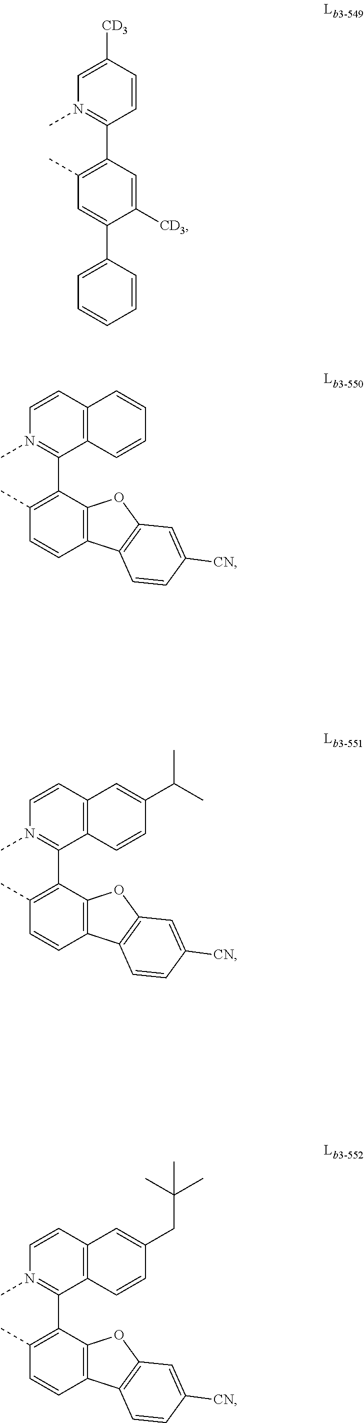 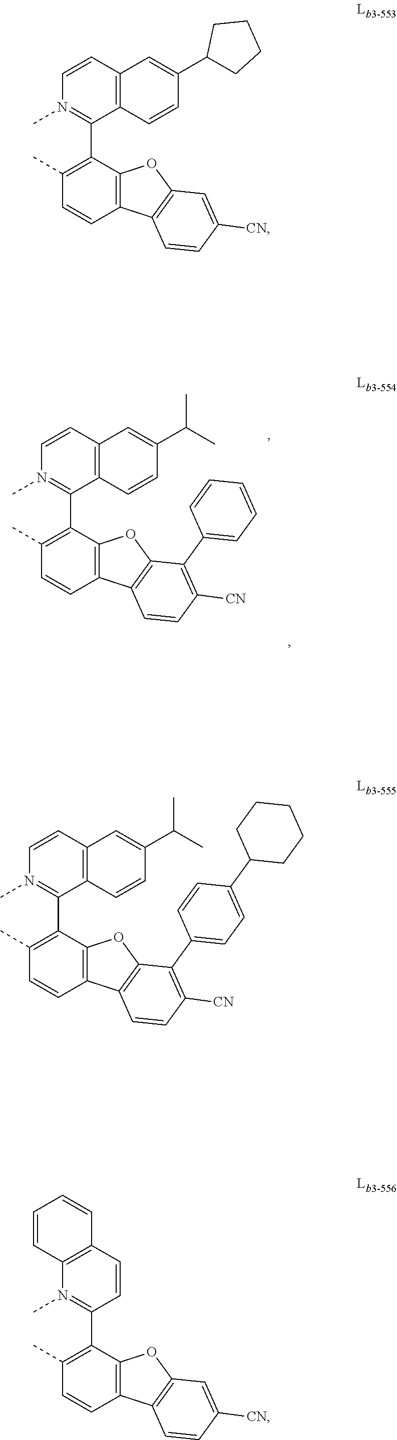 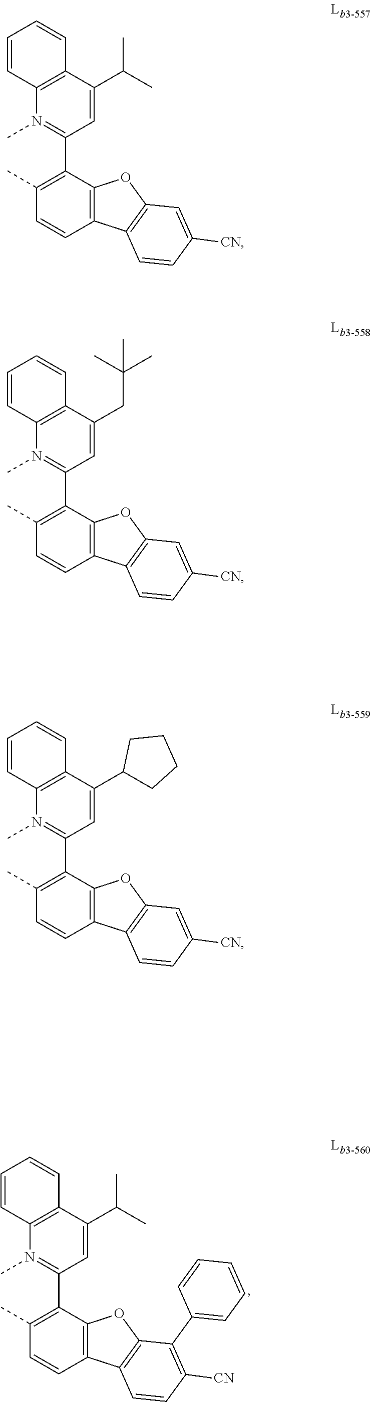 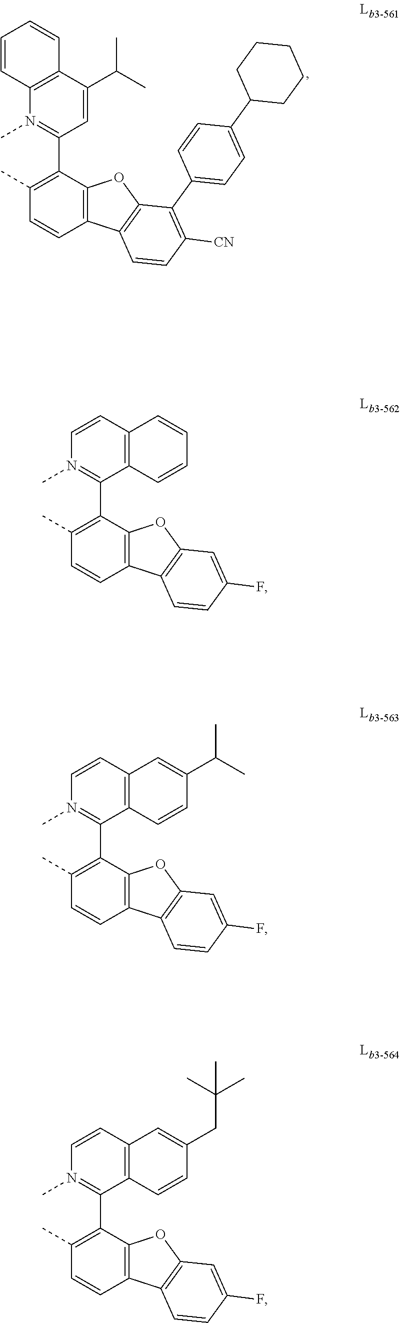 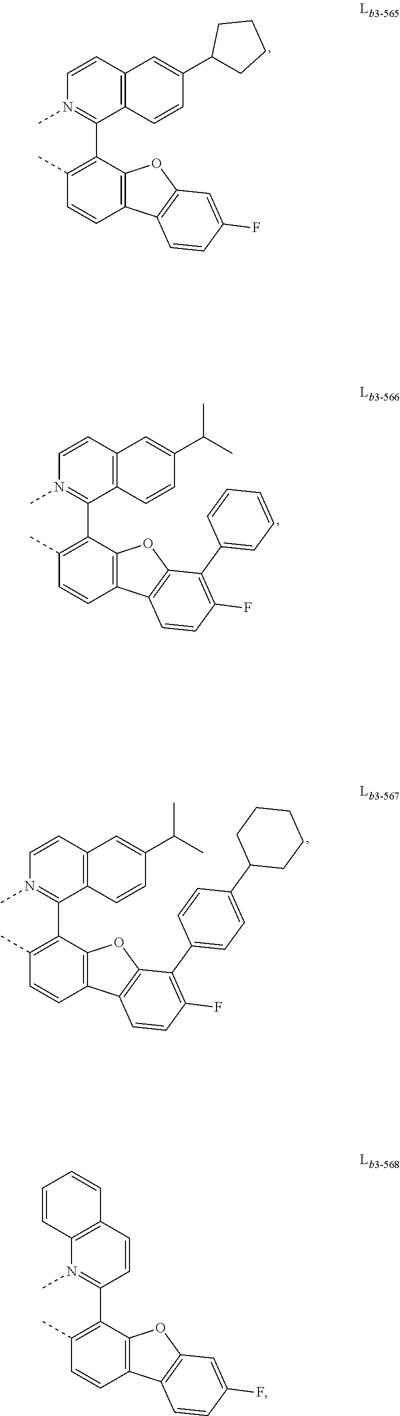 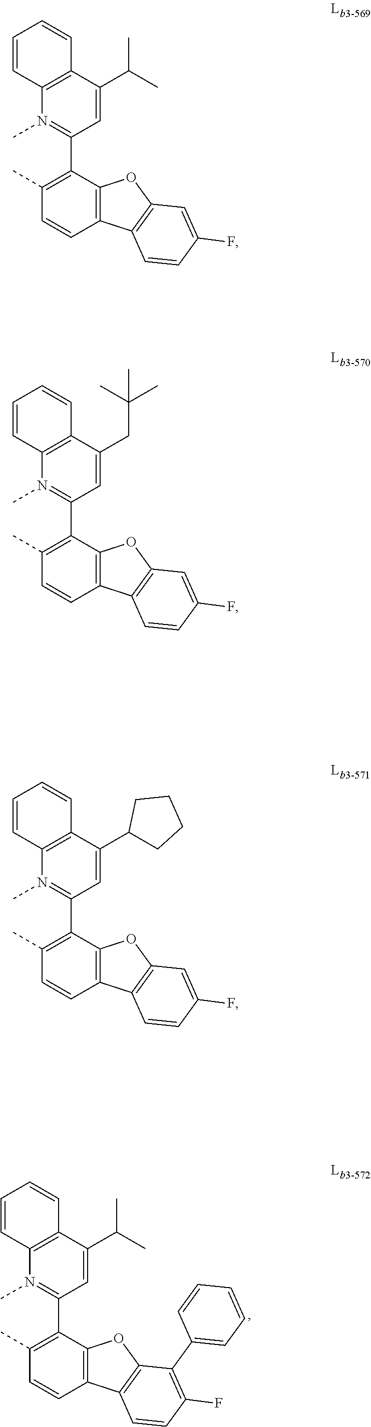 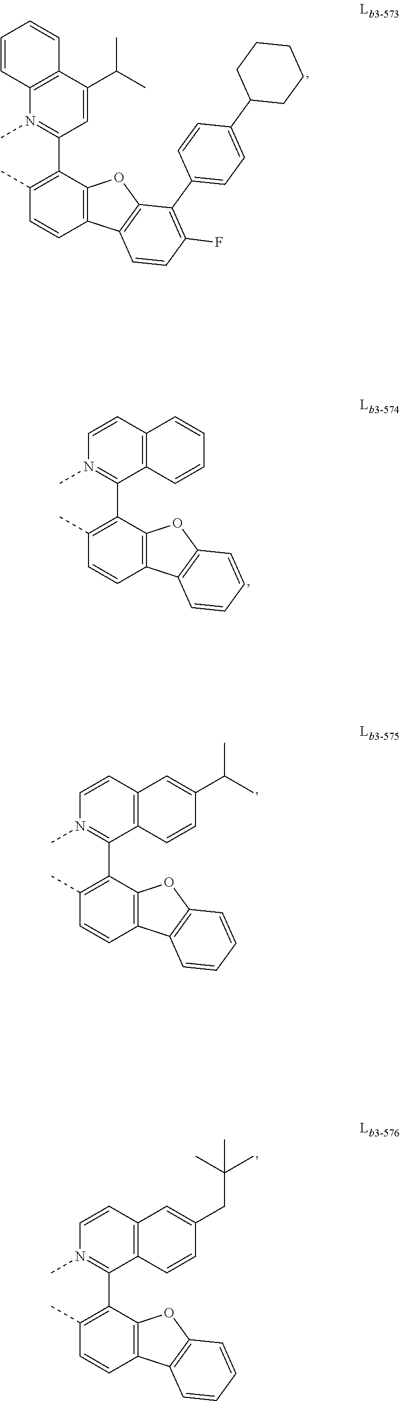 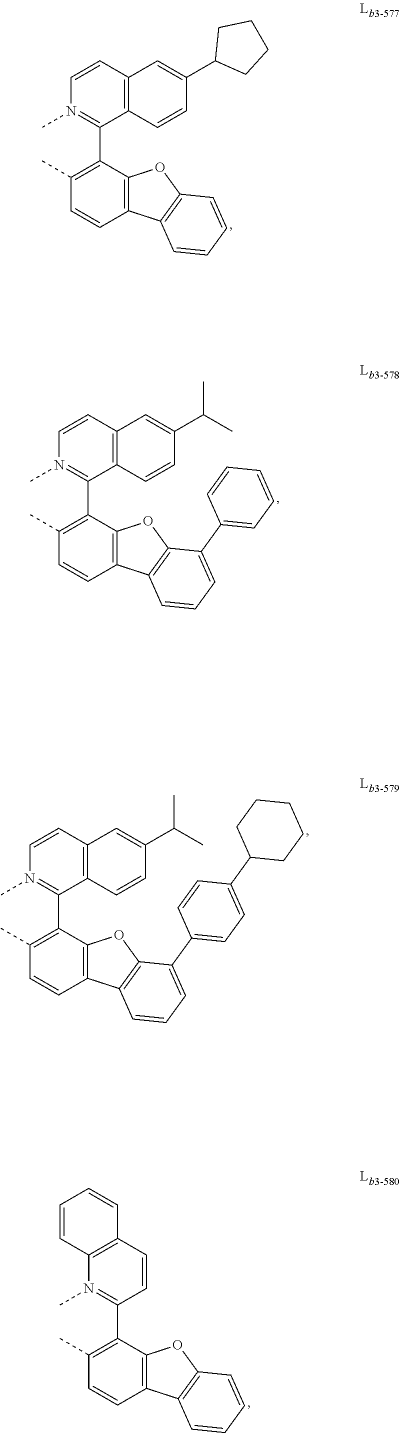 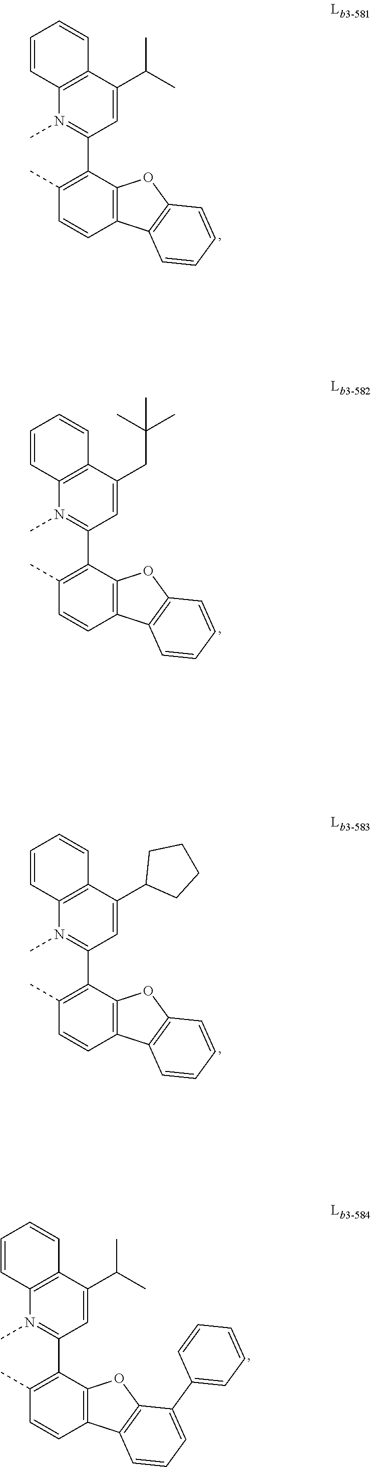 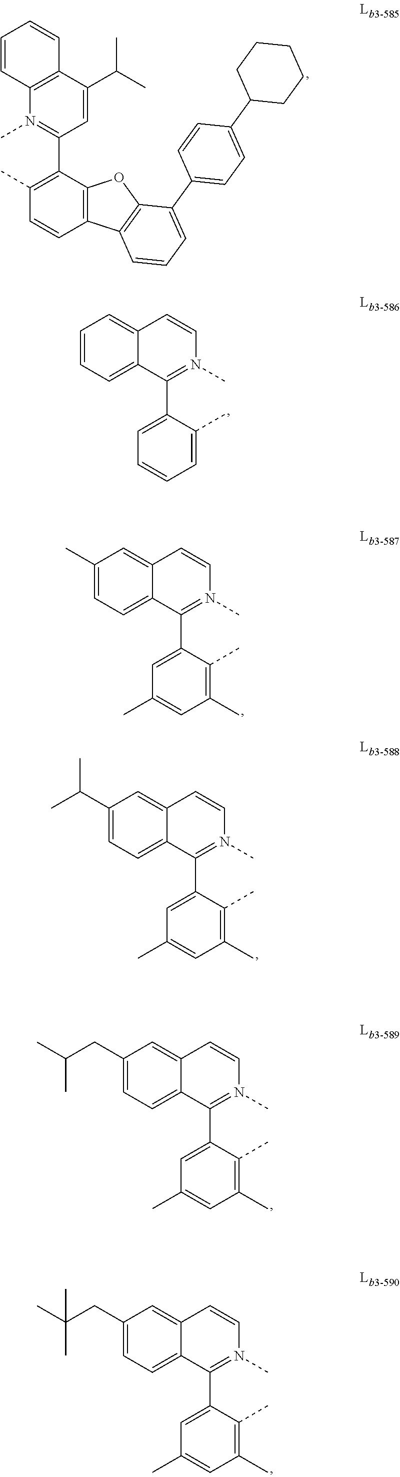 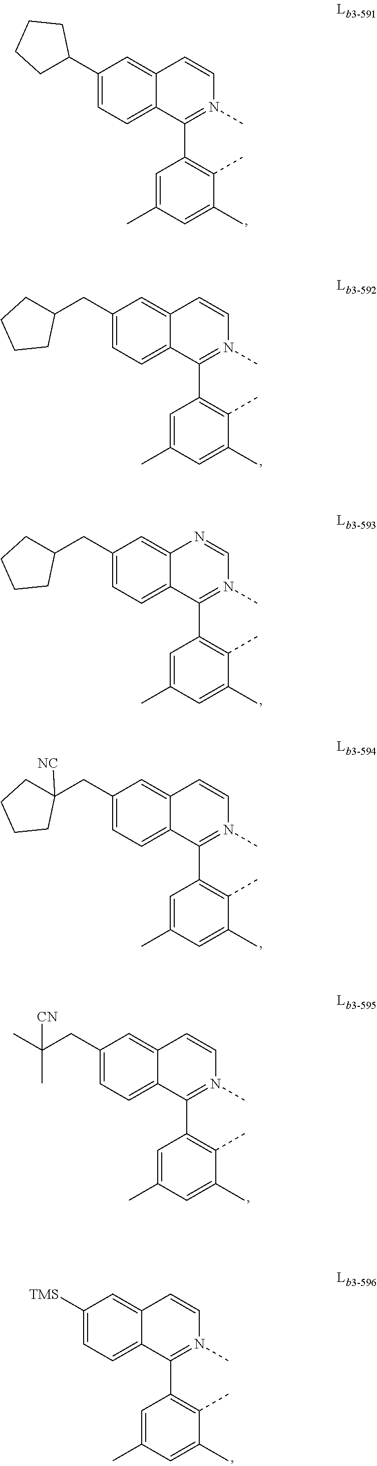 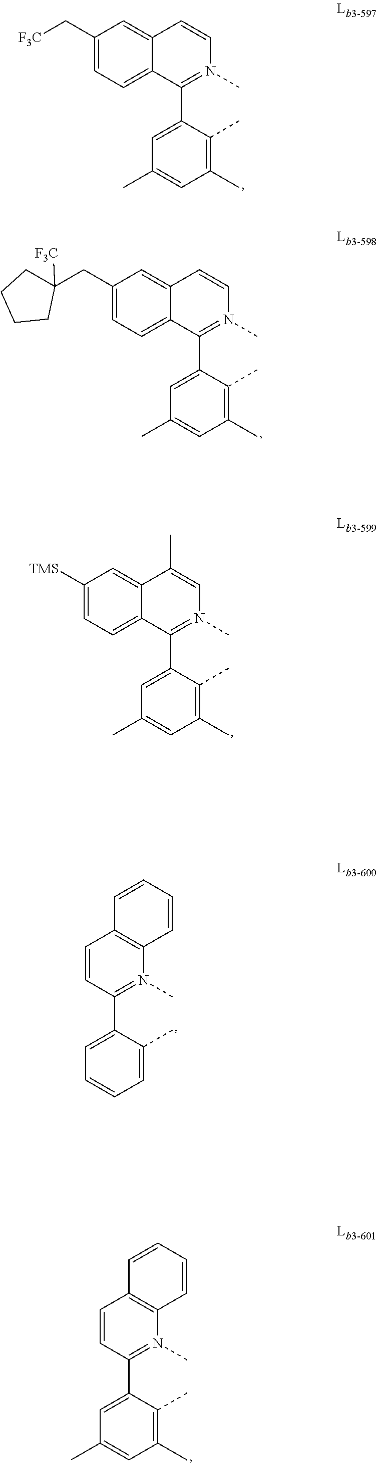 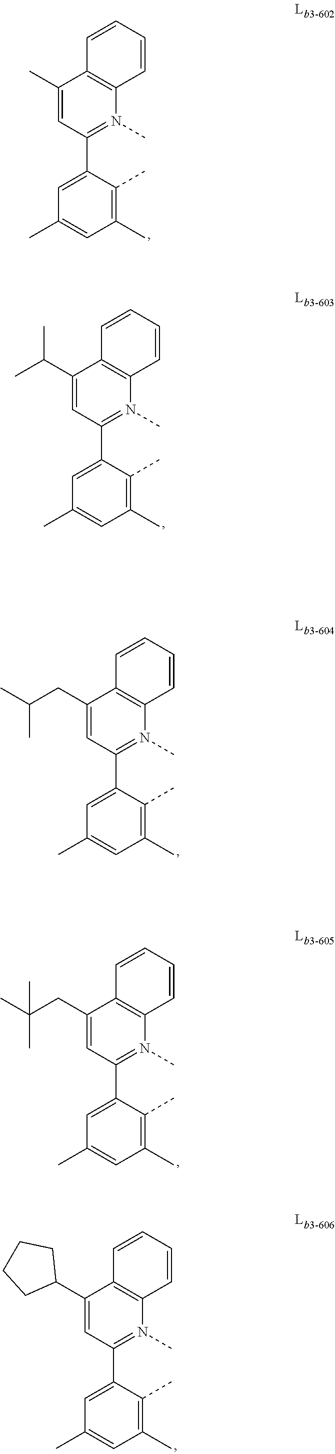 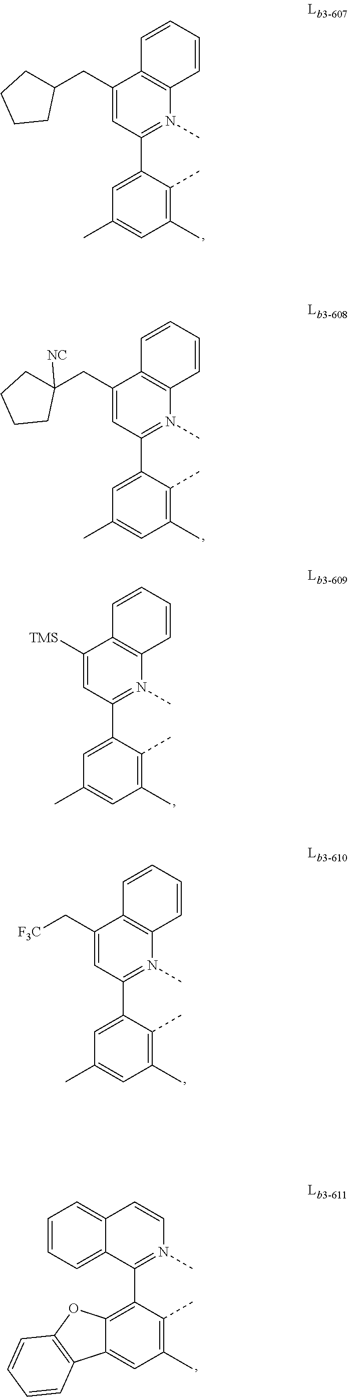 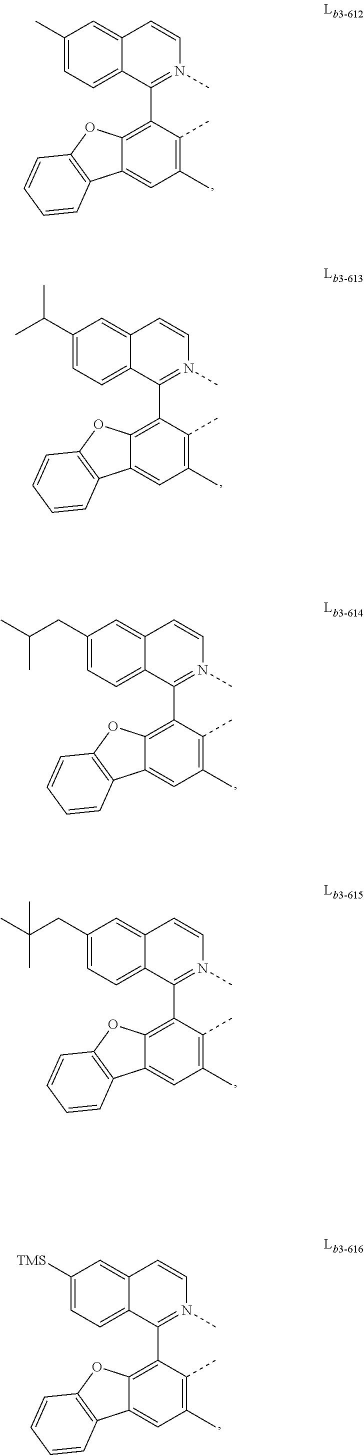 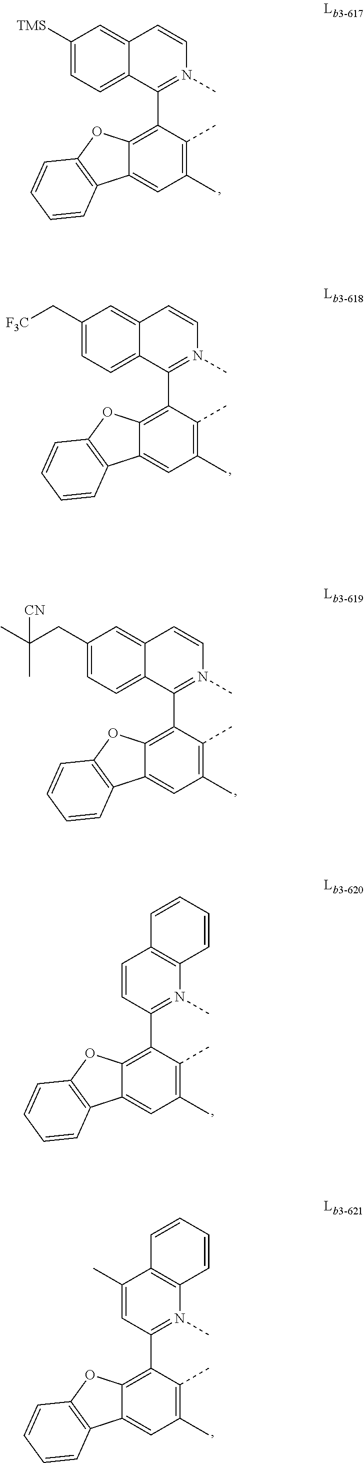 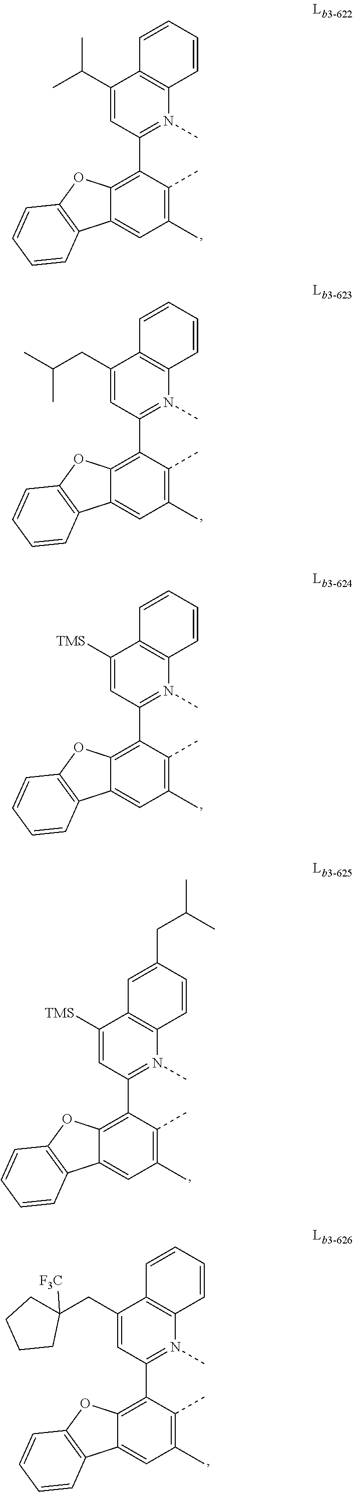 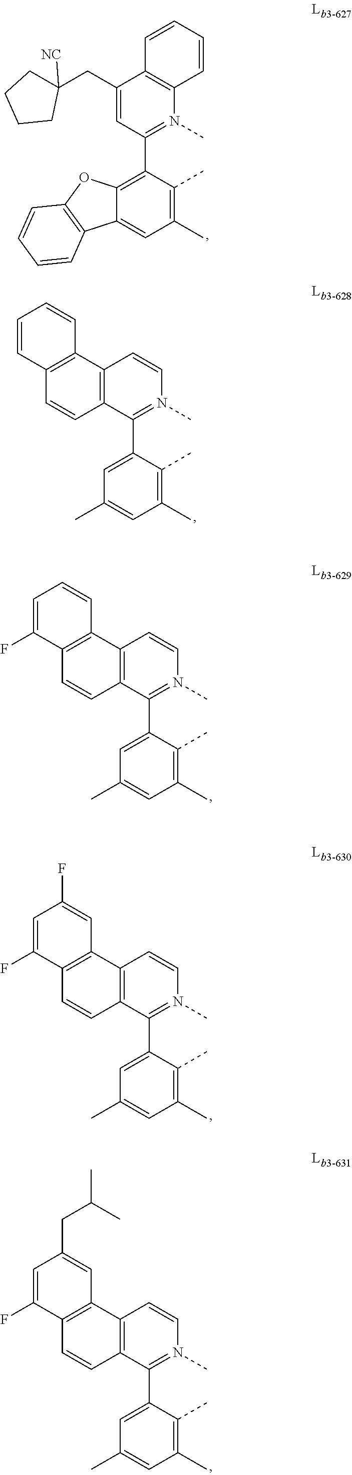 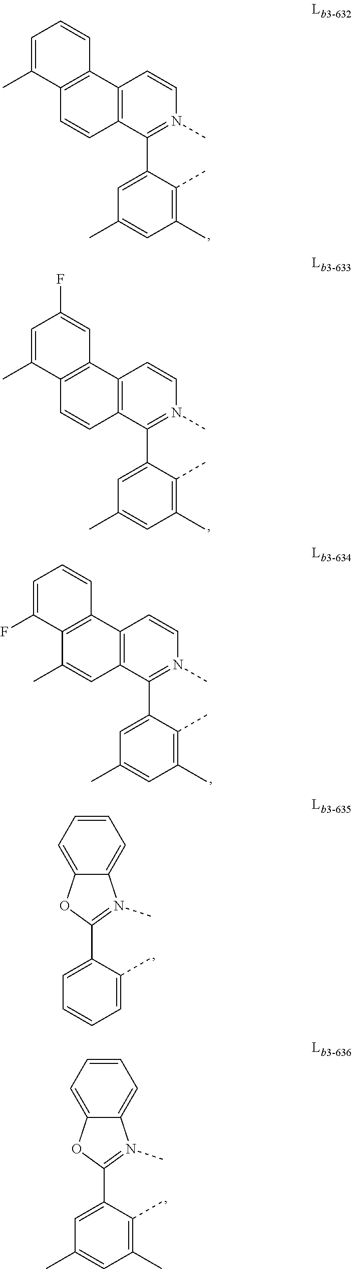 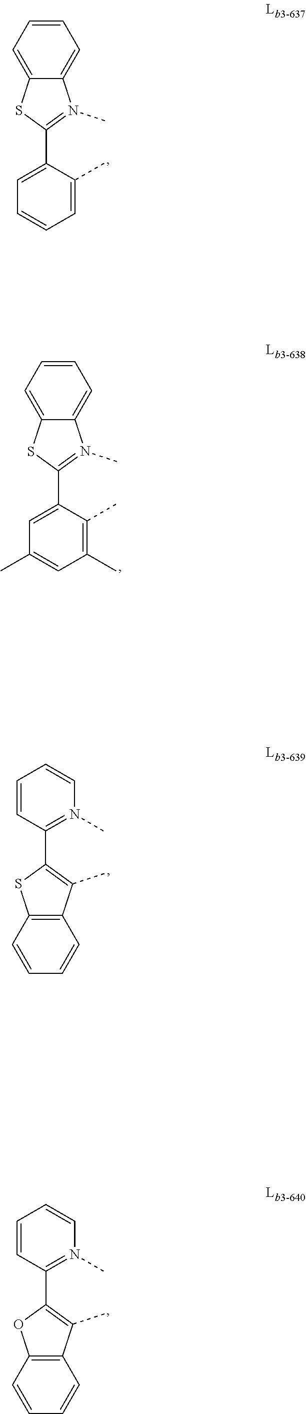 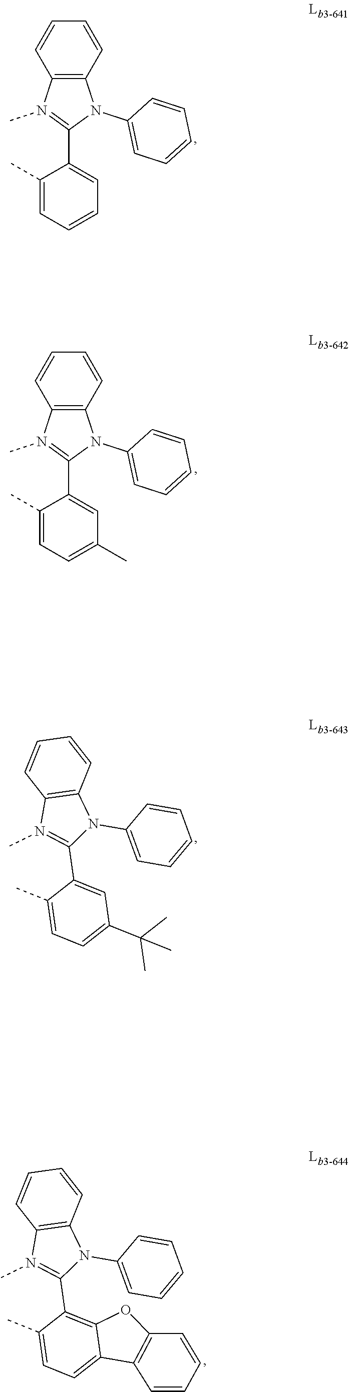 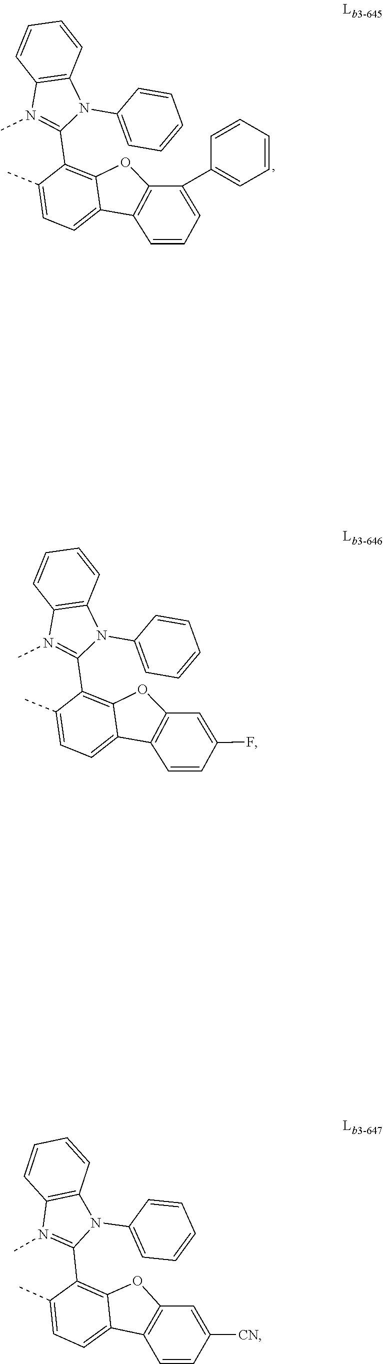 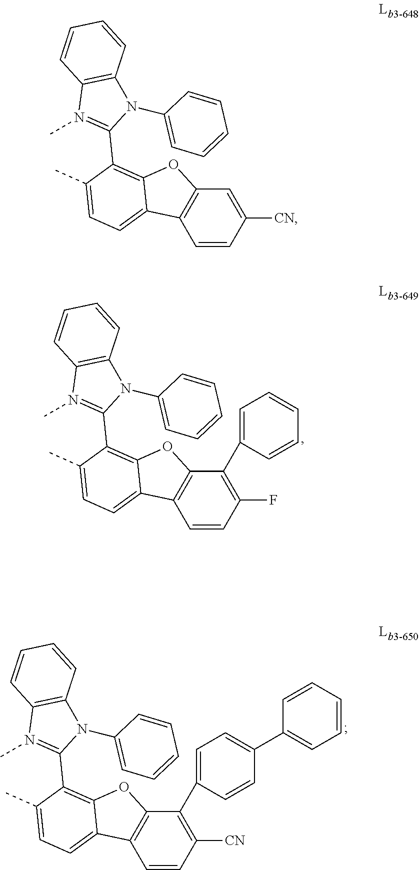
wherein optionally, hydrogen atoms in L_{b1-1} to L_{b1-355}, L_{b2-1} to L_{b2-261} and L_{b34} to L_{b3-650} can be partially or fully substituted with deuterium atoms.

19. The metal complex according to claim 18, wherein the metal complex has a structure of Ir(Lₐ)(L_{b})(L_{c}), wherein Lₐ is selected from the group consisting of Lₐ₁₋₁ to Lₐ₁₋₂₃₁, Lₐ₂₋₁ to Lₐ₂₋₁₆₁, Lₐ₃₋₁ to Lₐ₃₋₁₃₀, L_{b} is selected from the group consisting of L_{b1-1} to L_{b1-355}, L_{b24} to L_{b2-261}, L_{b3-1} to L_{b3-650}, and L_{c} is, at each occurrence identically or differently, selected from the group consisting of the following:

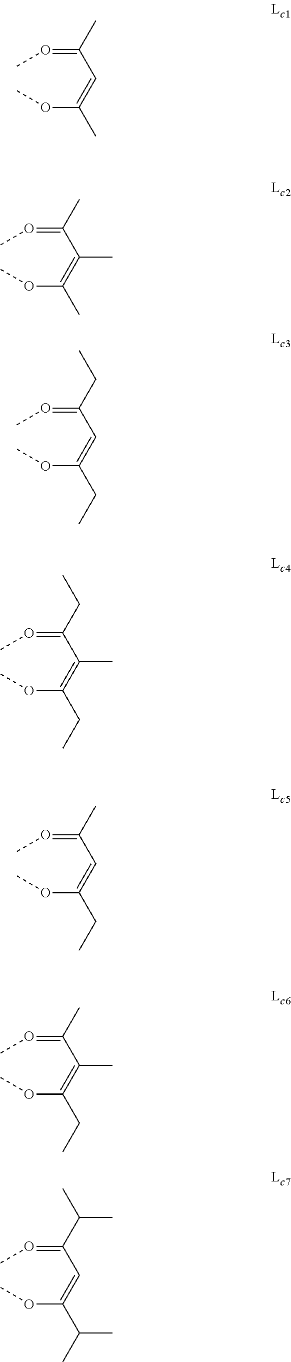

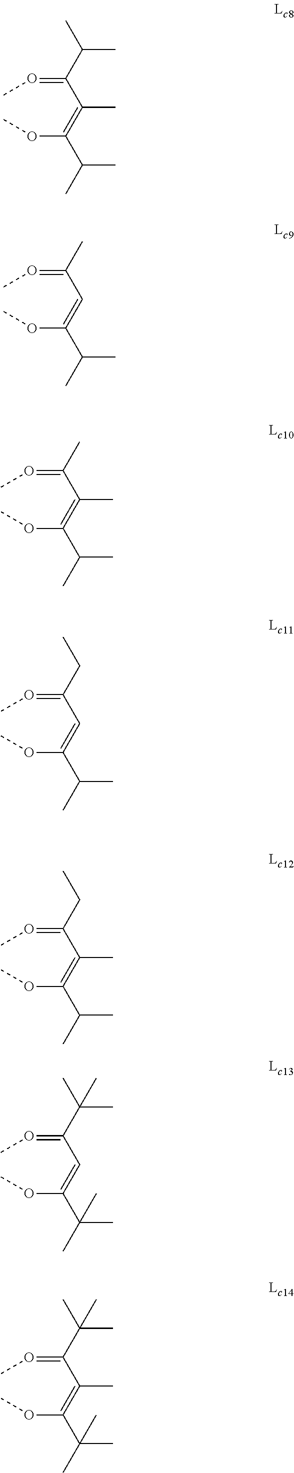

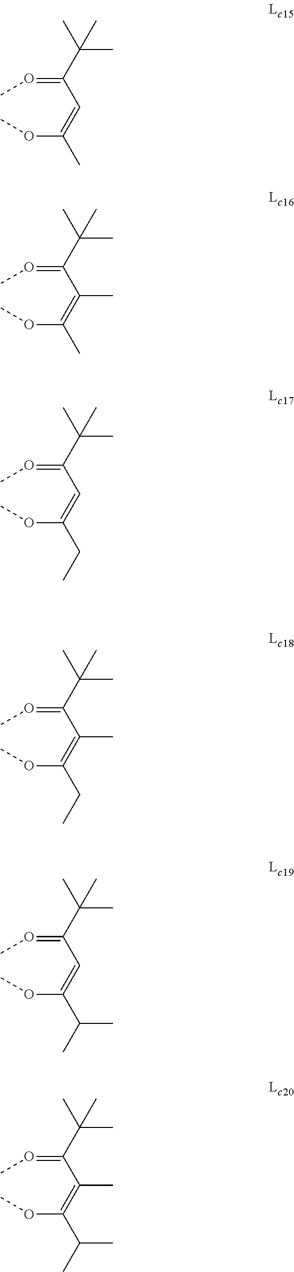

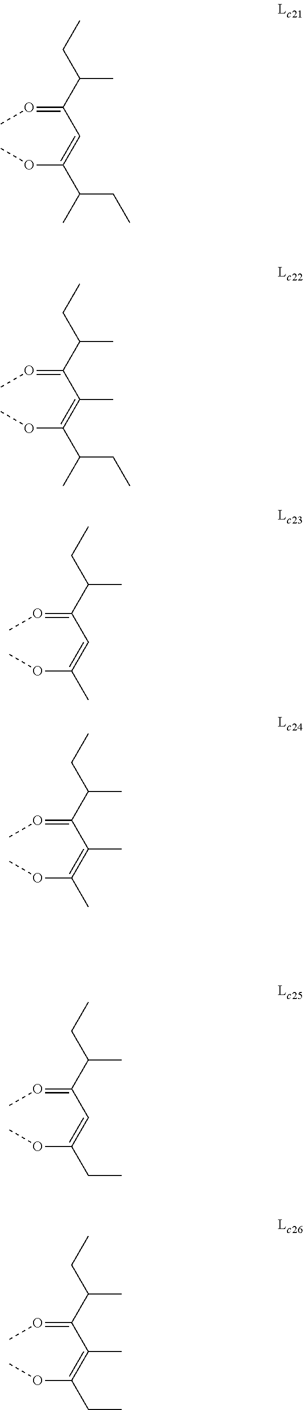

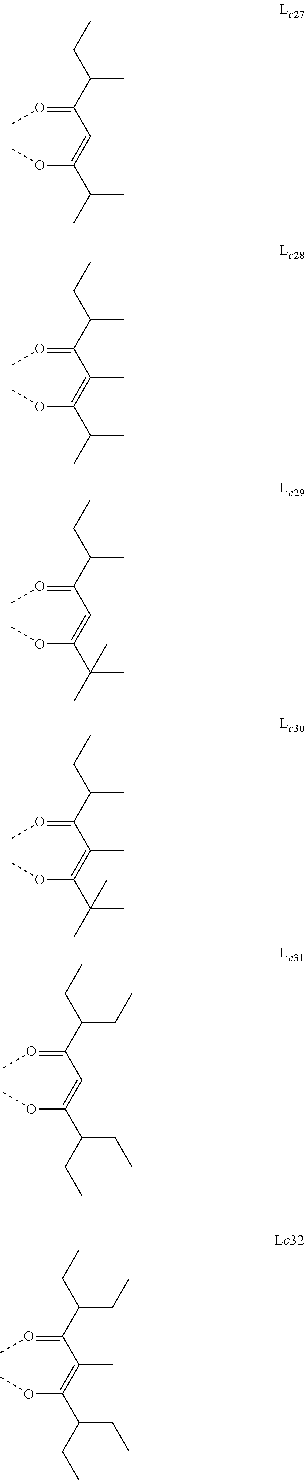

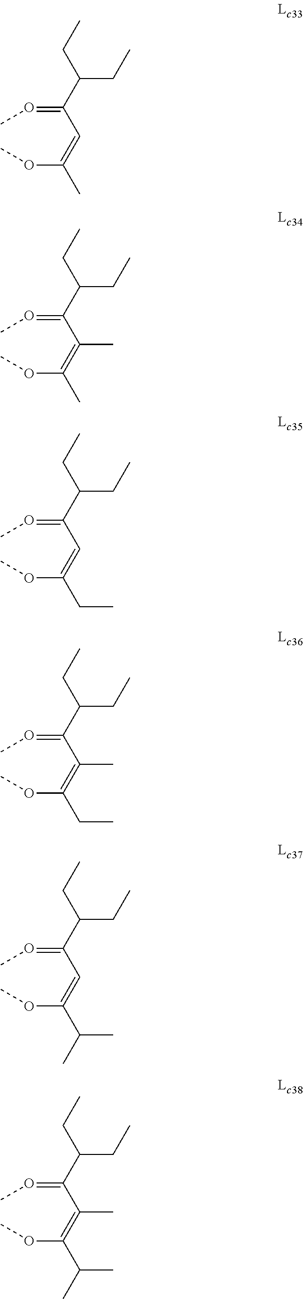

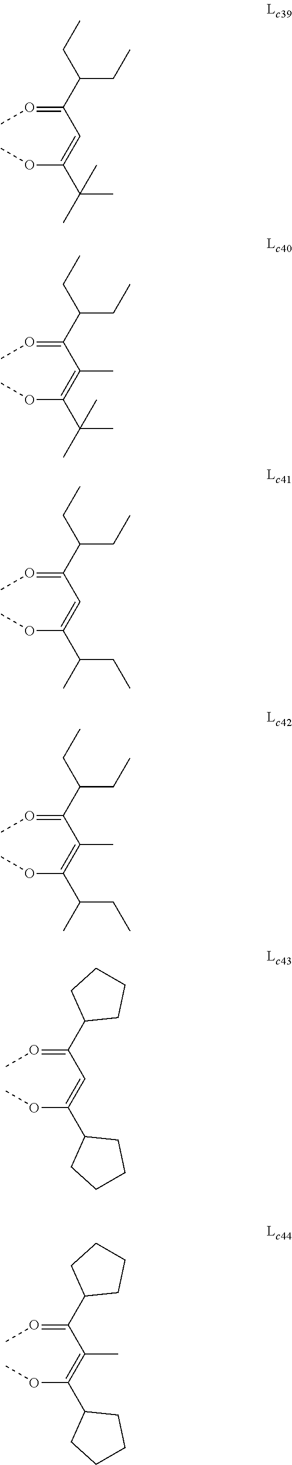

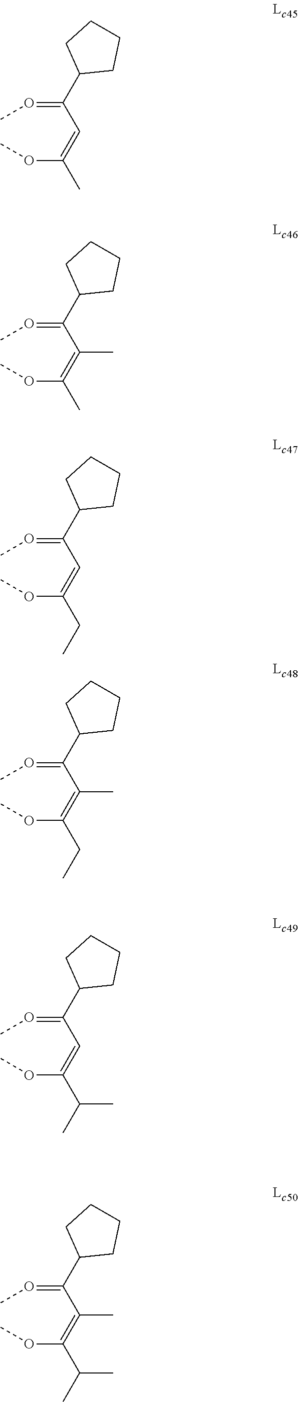

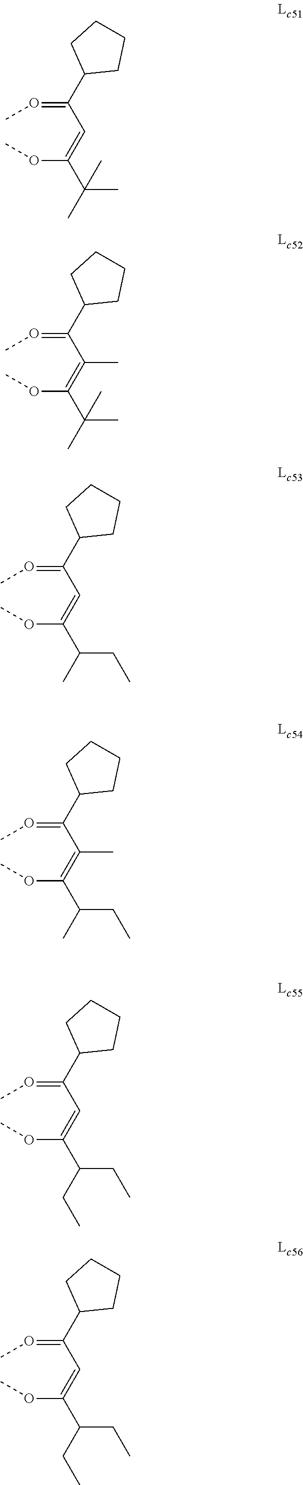

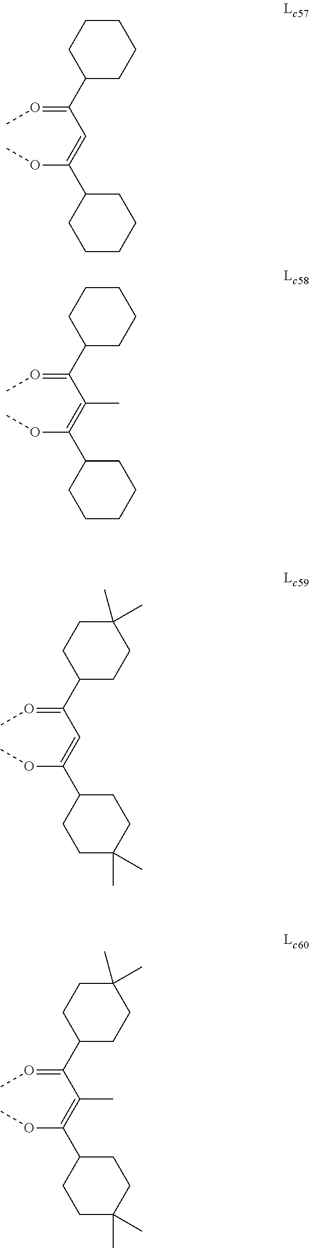

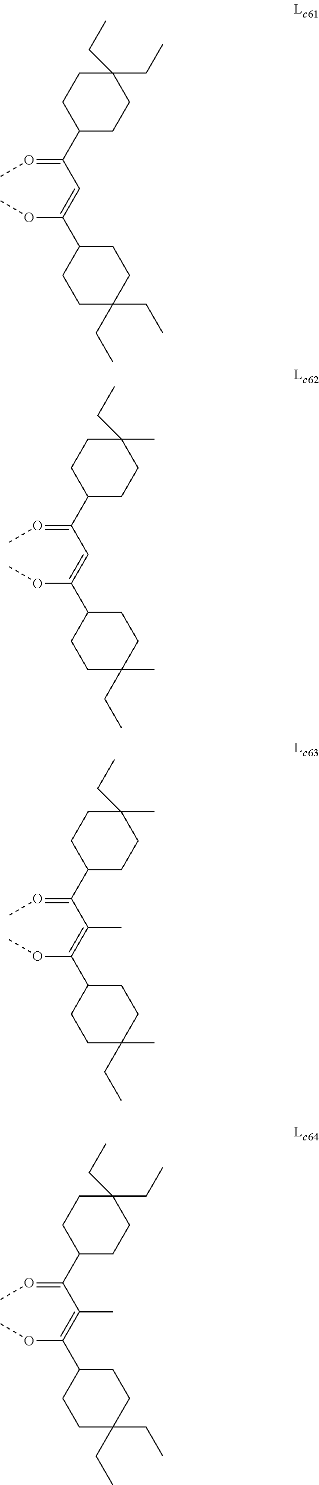

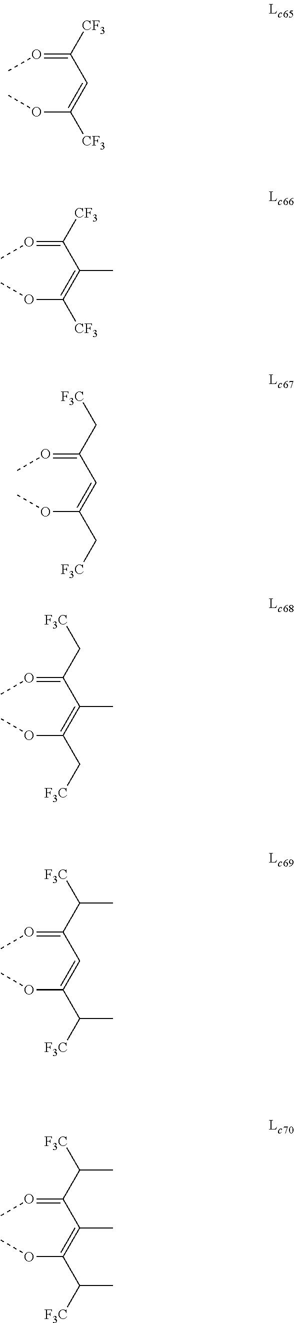

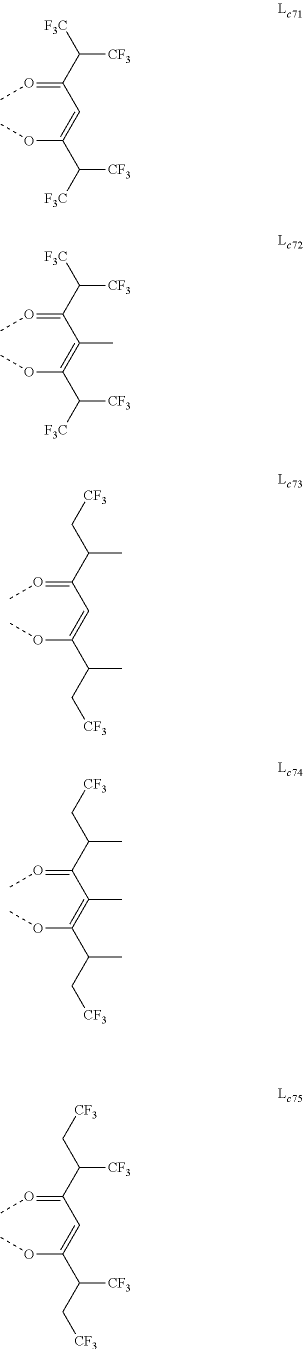

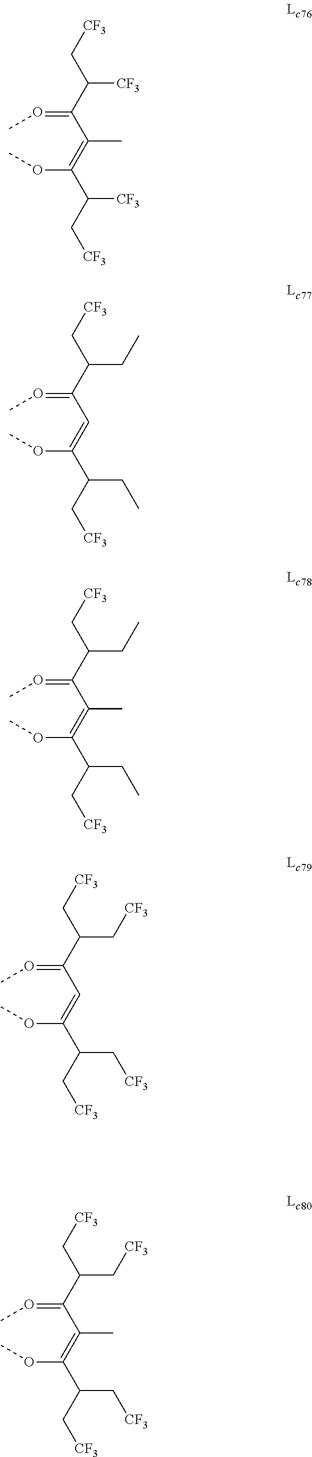

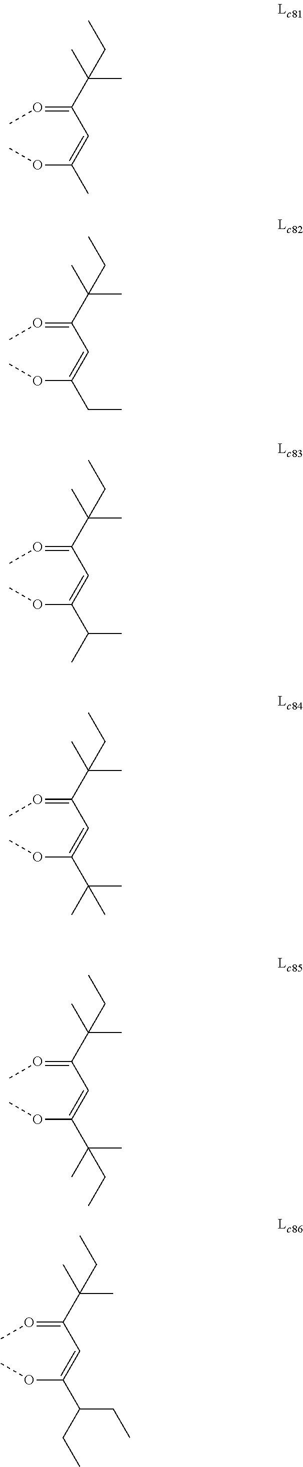

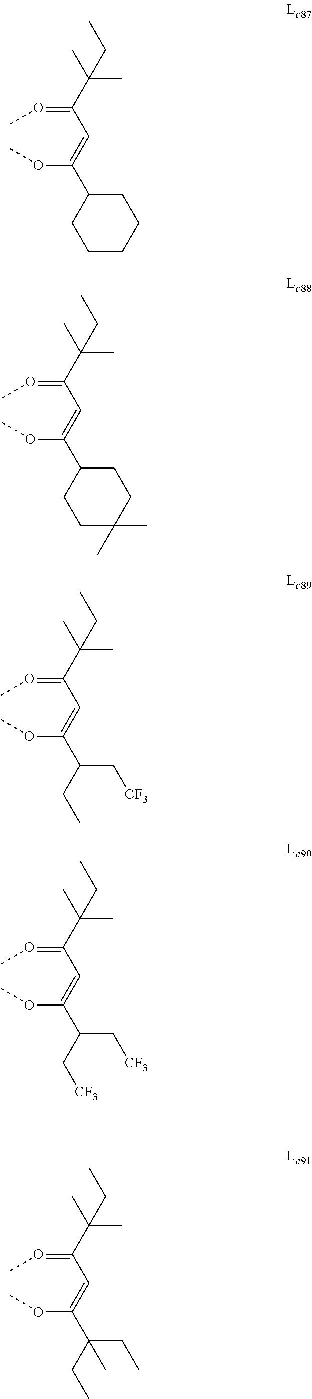

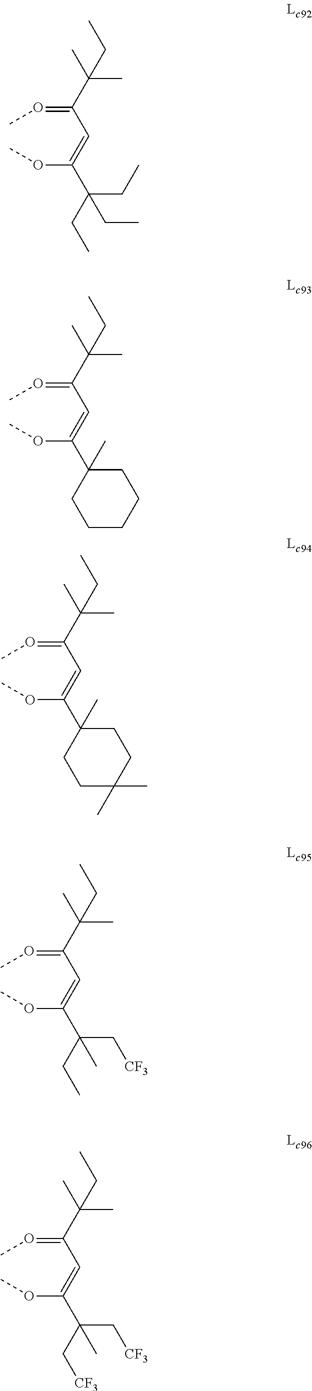

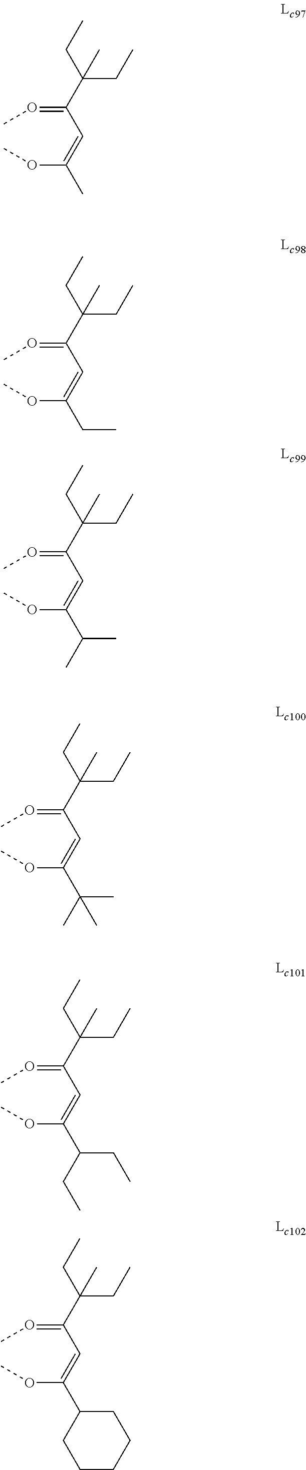

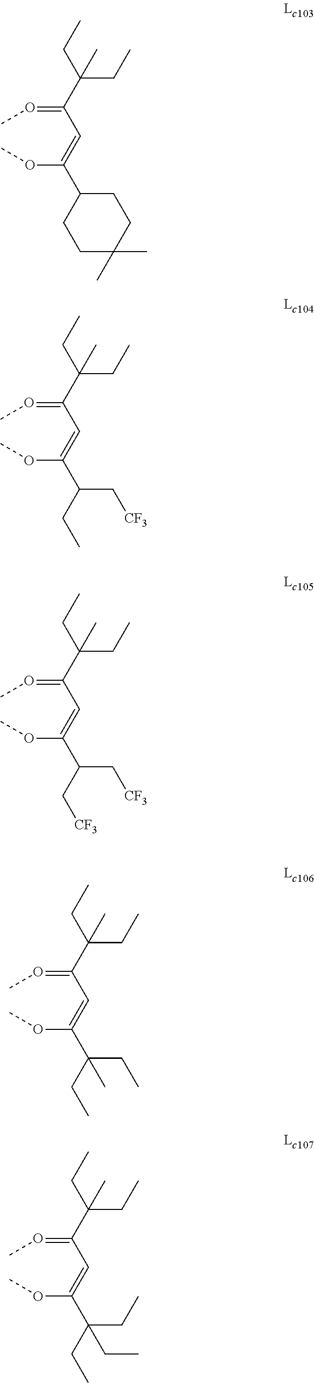

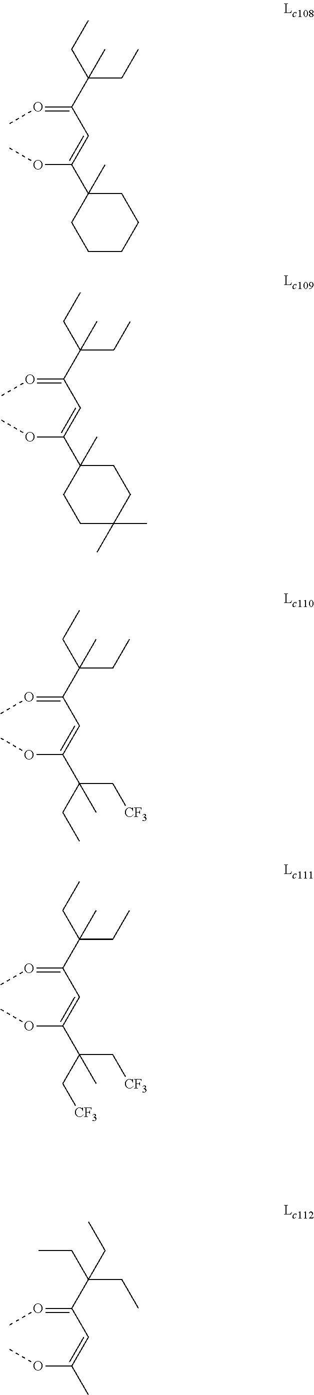

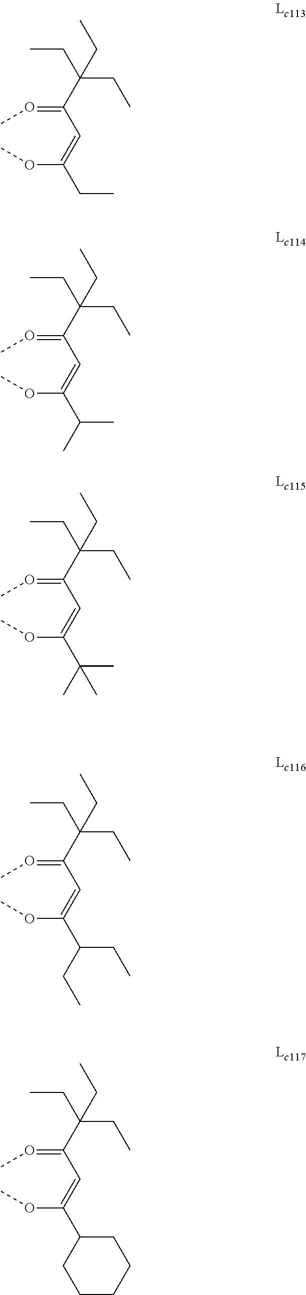

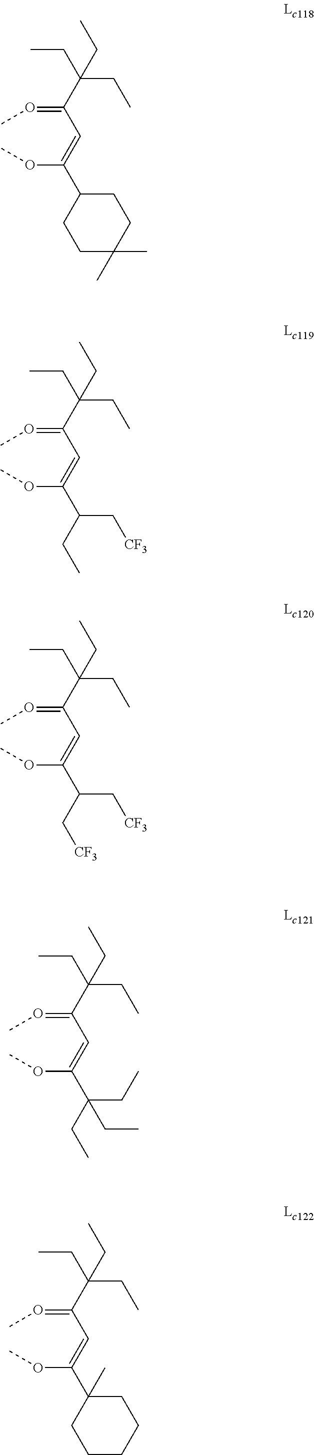

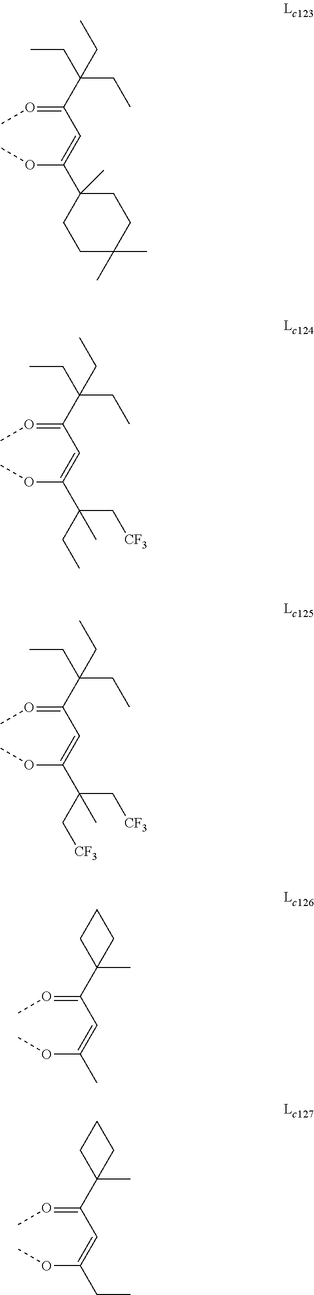

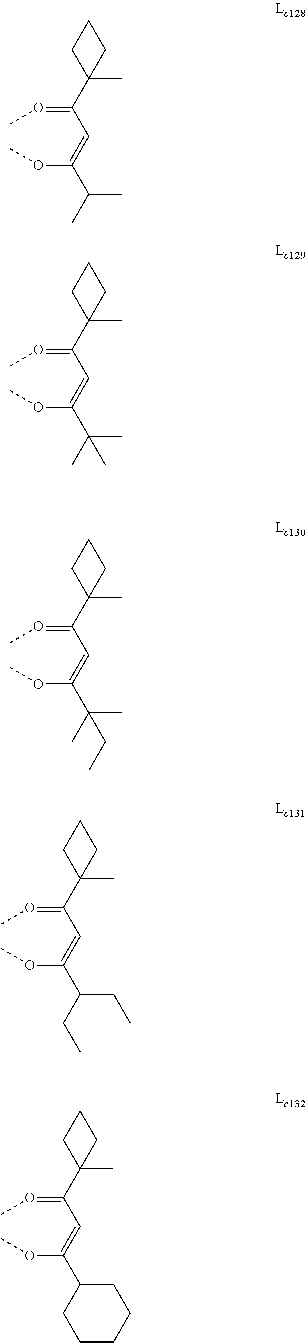

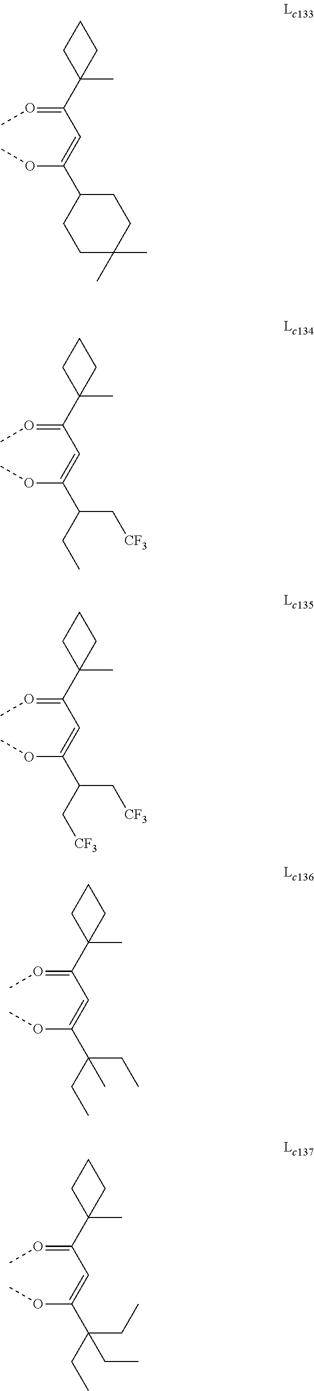

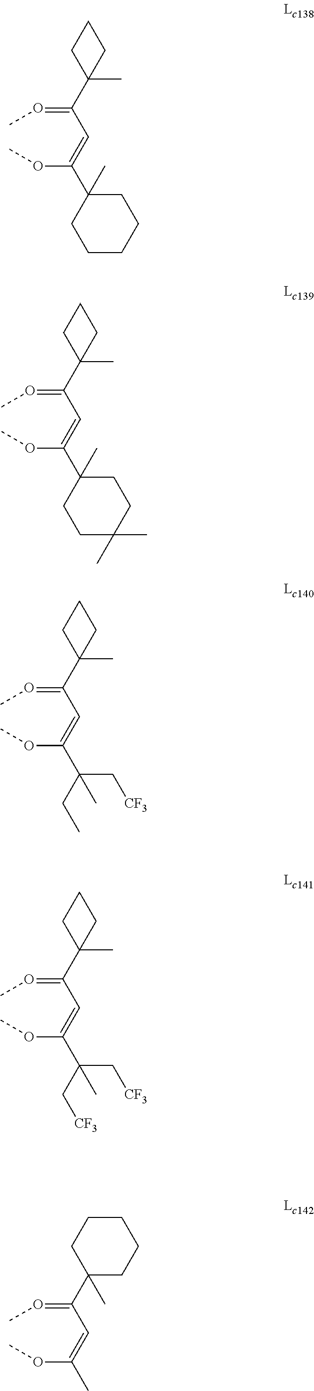

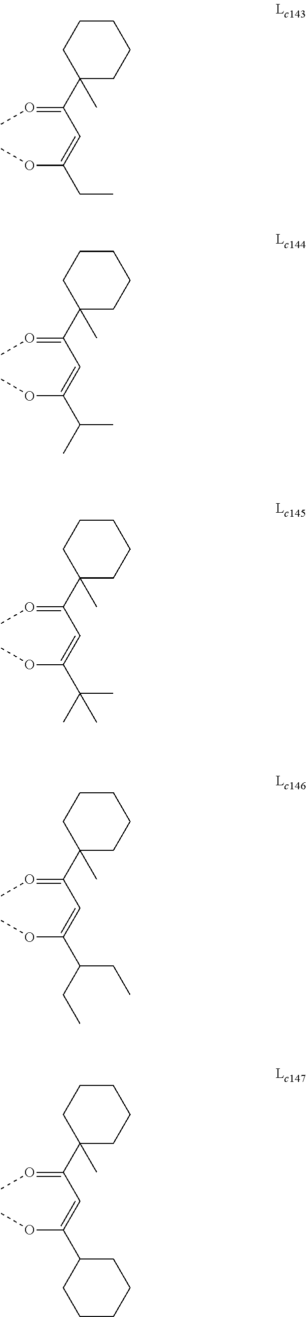

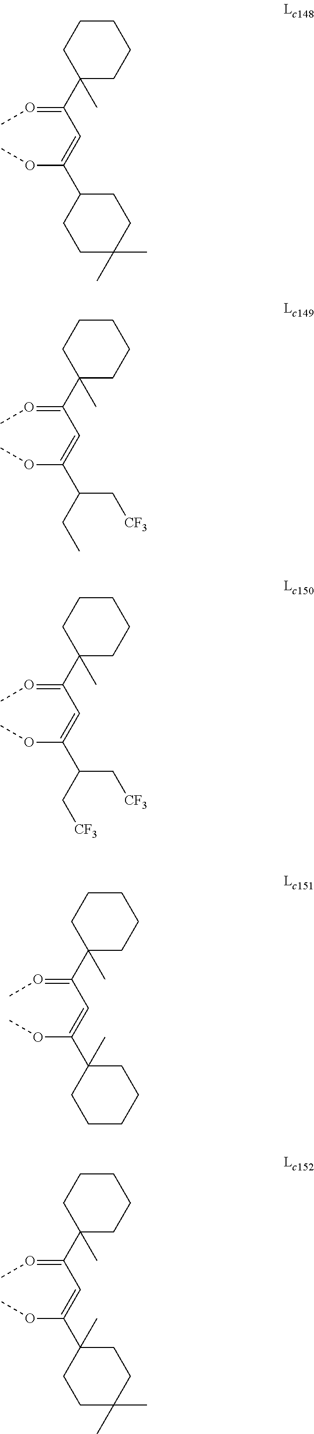

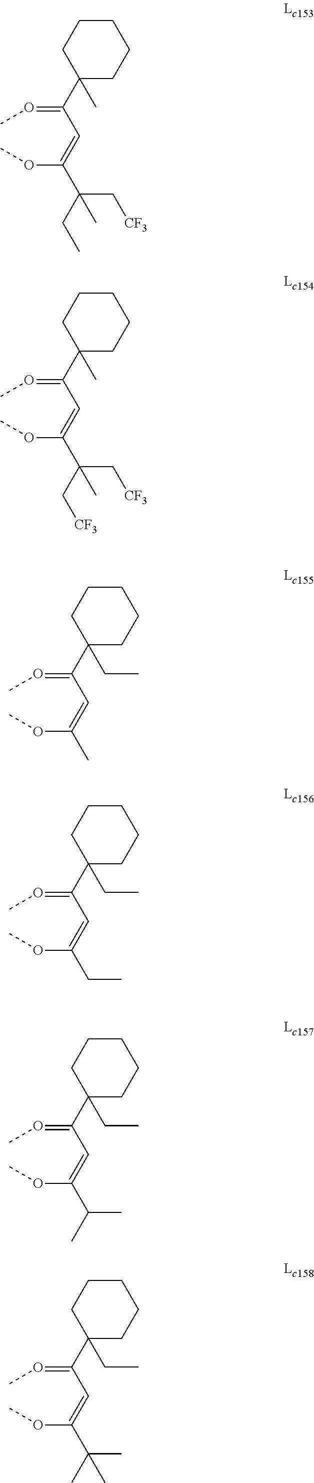

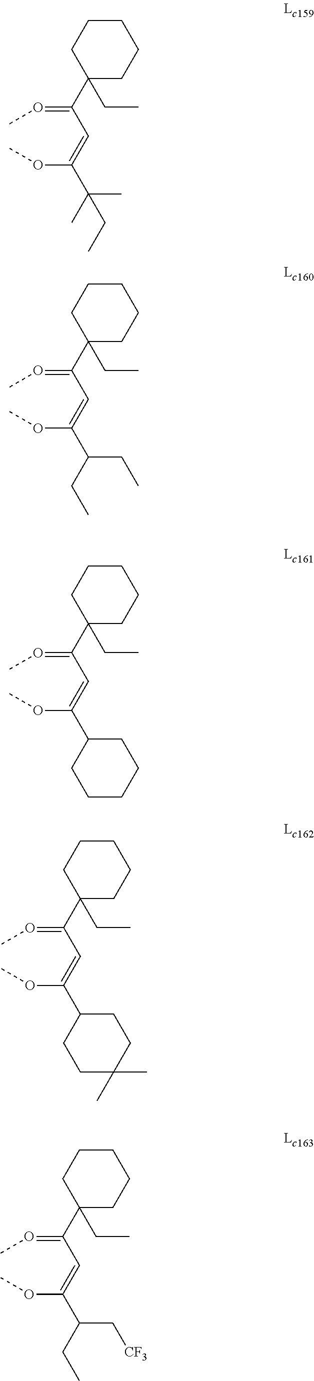

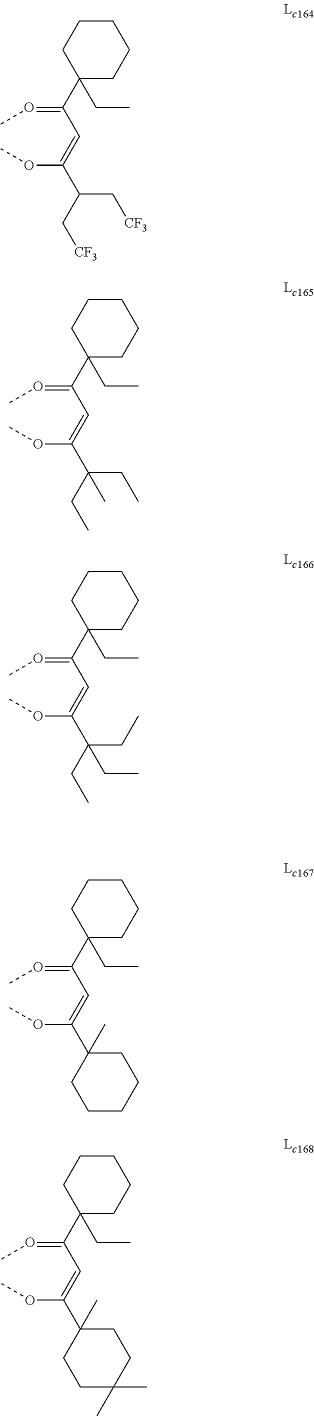

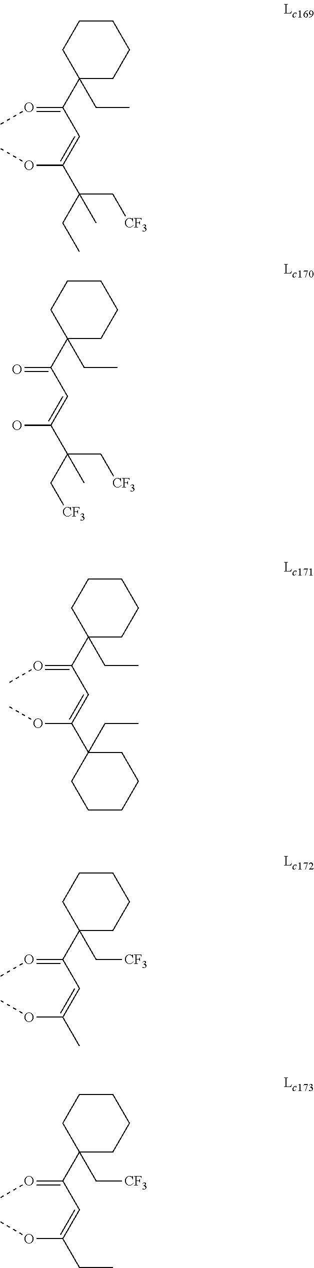

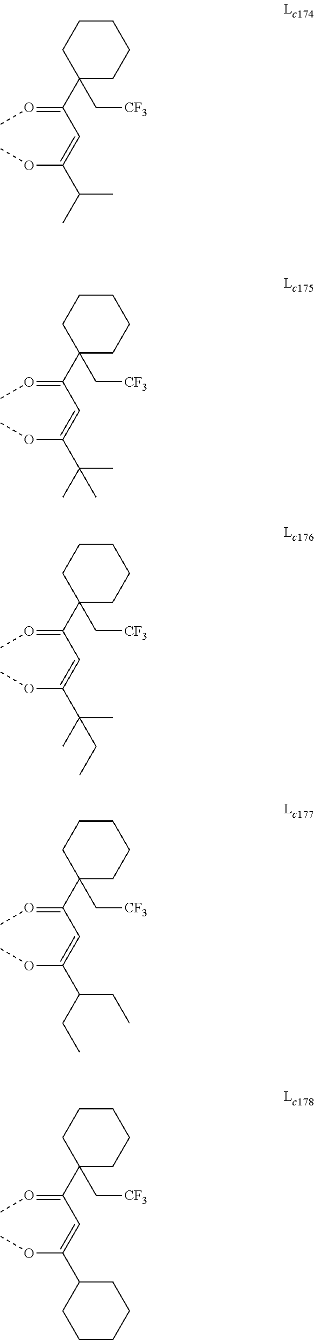

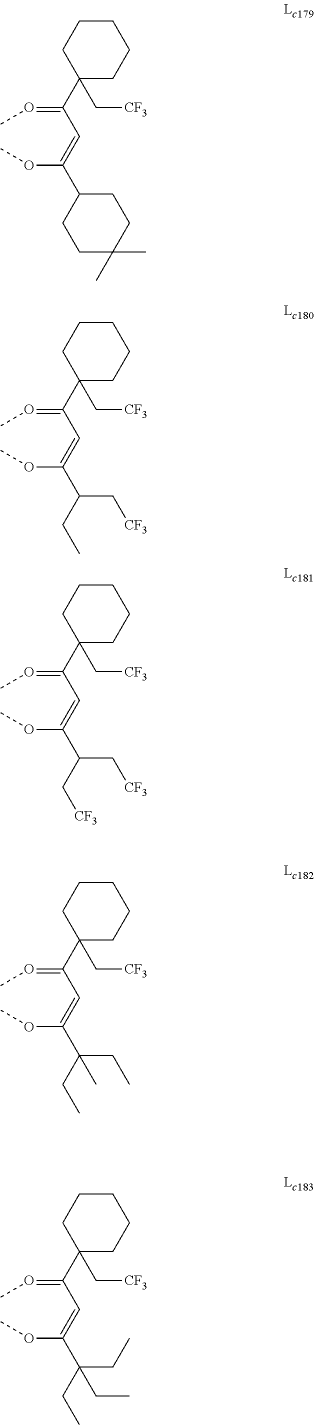

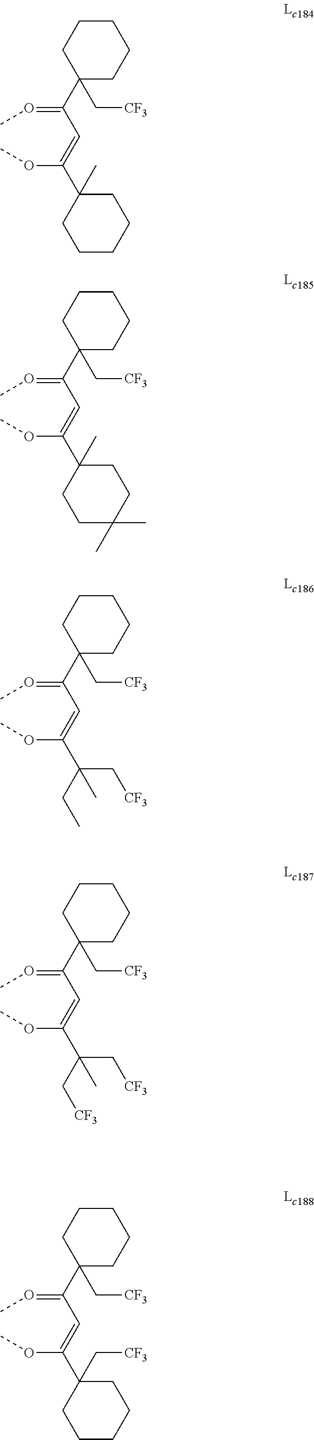

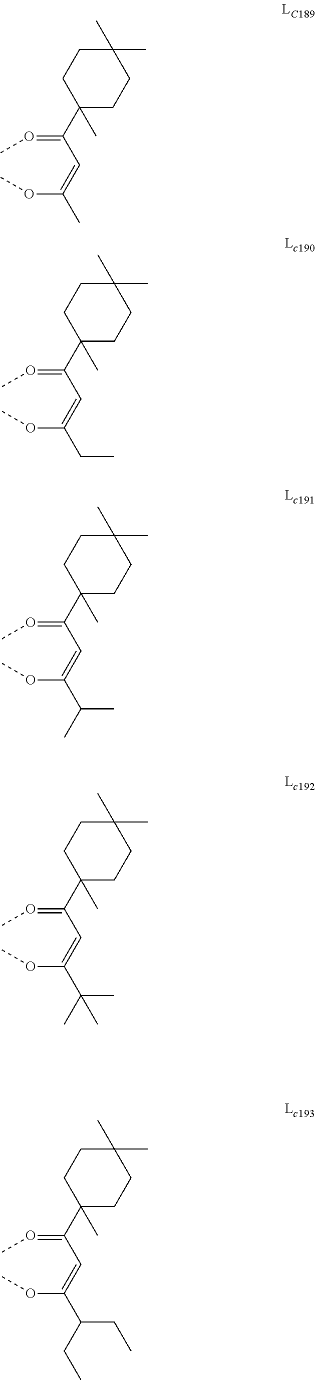

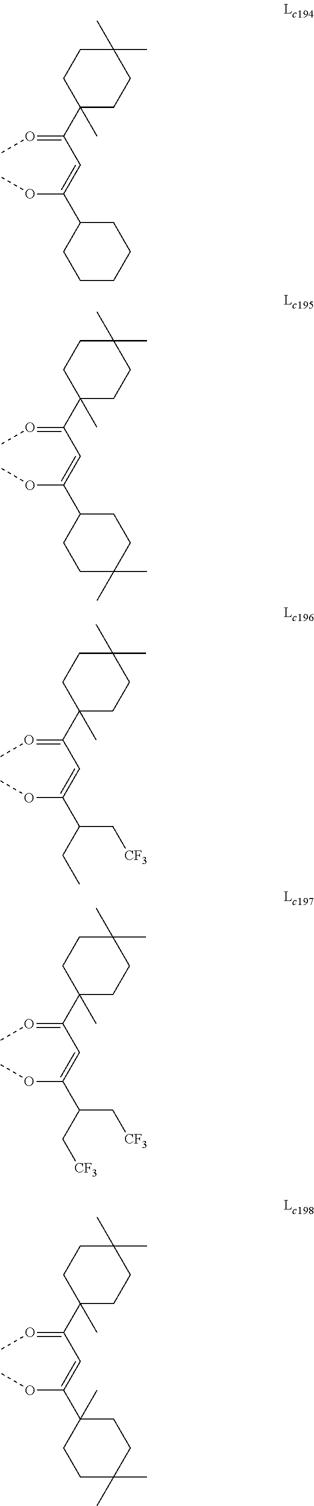

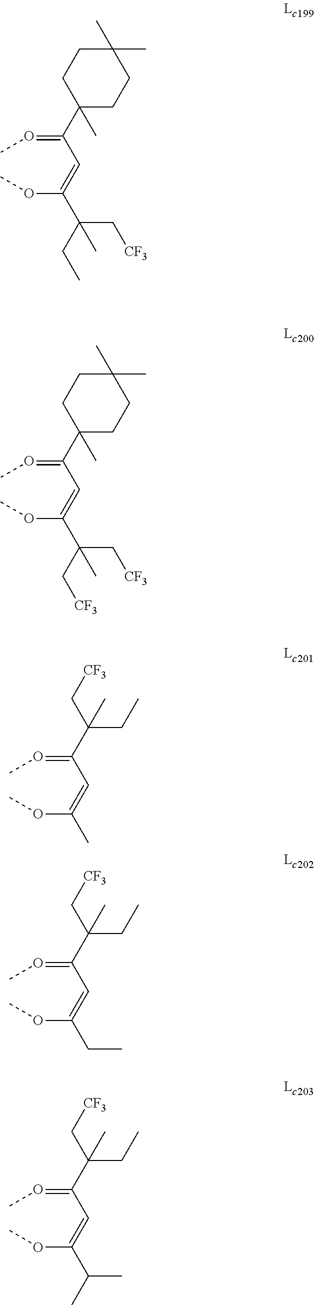

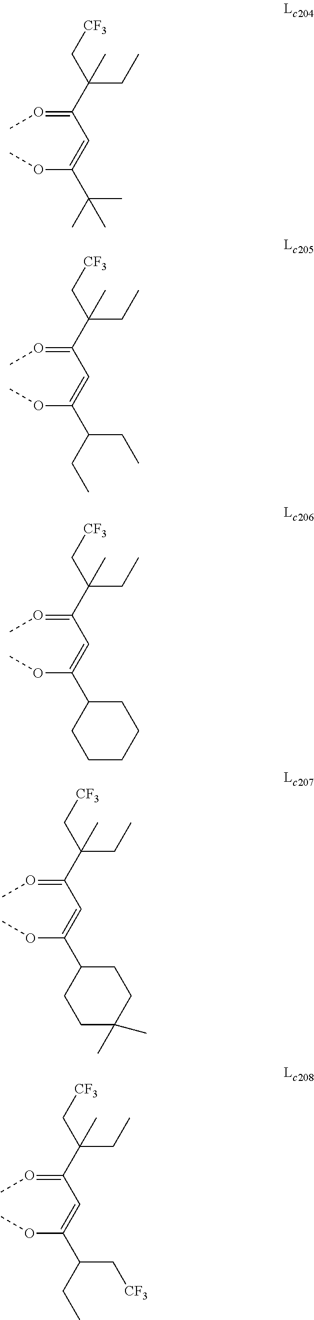

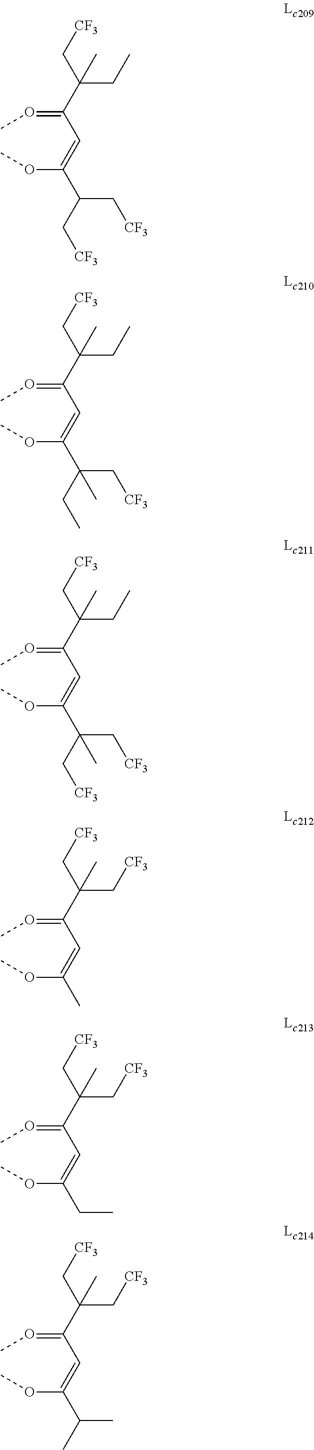

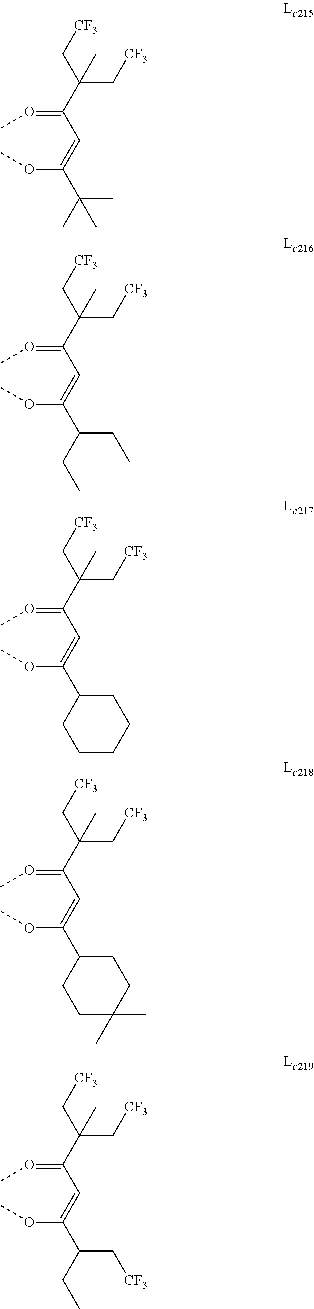

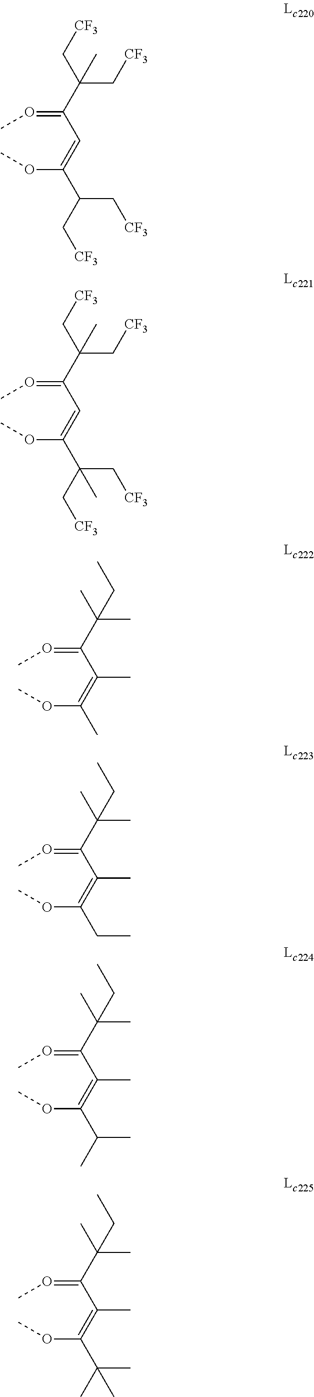

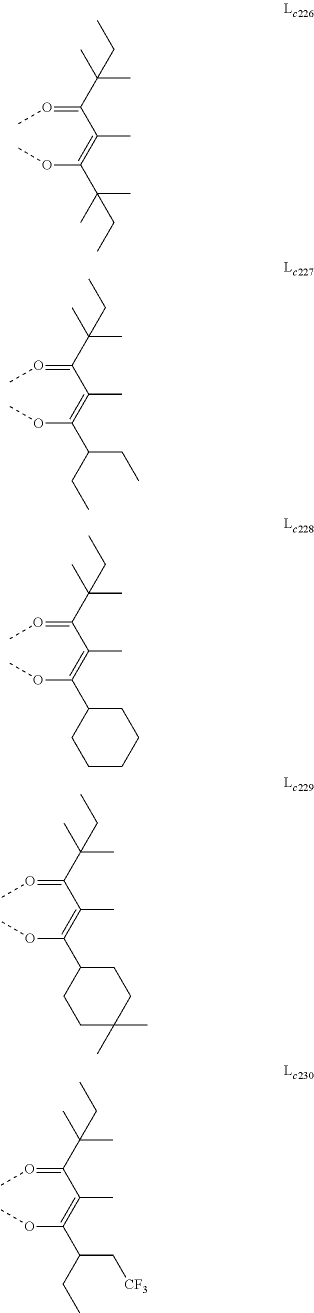

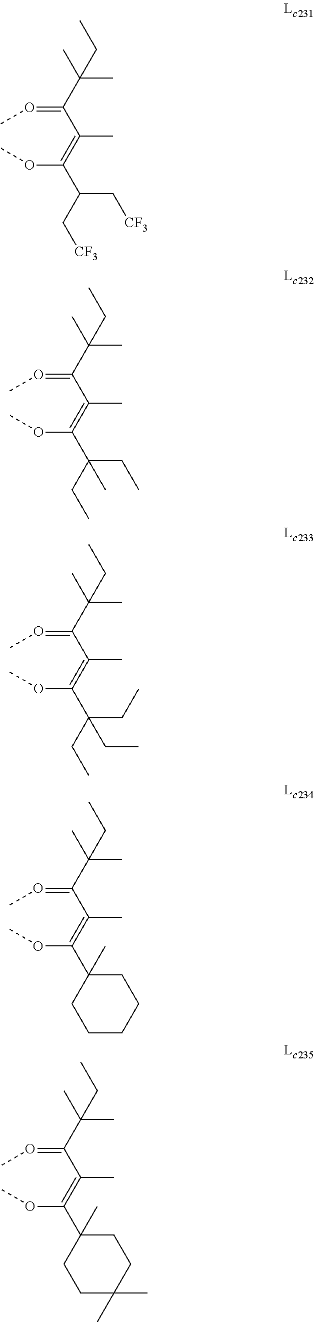

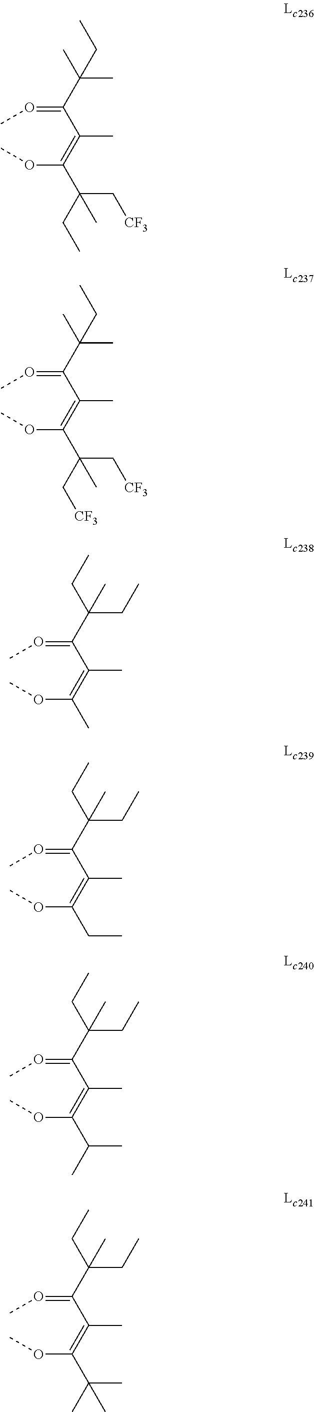

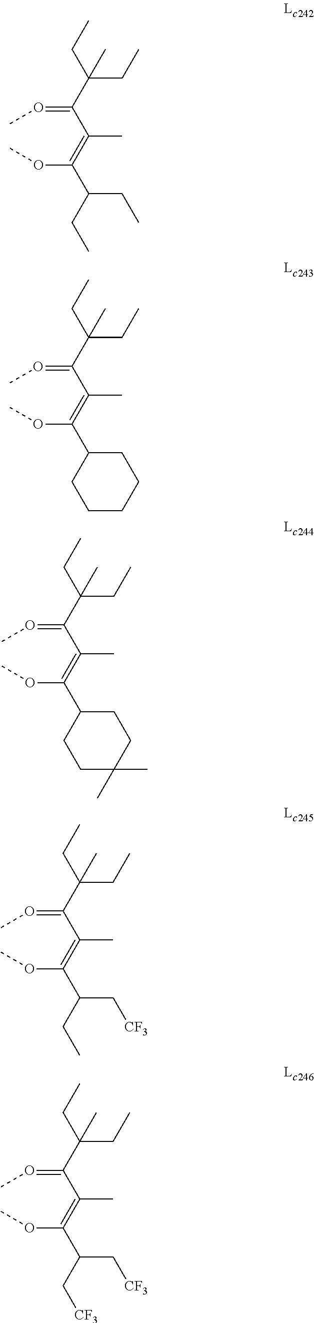

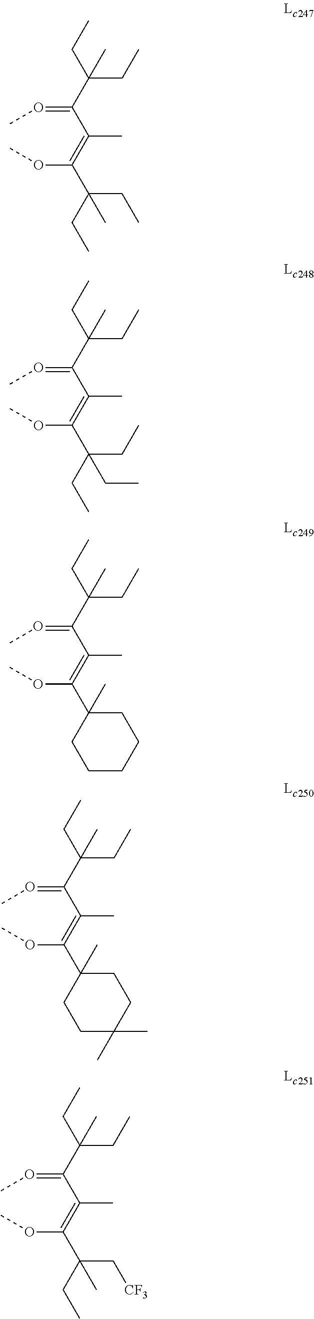

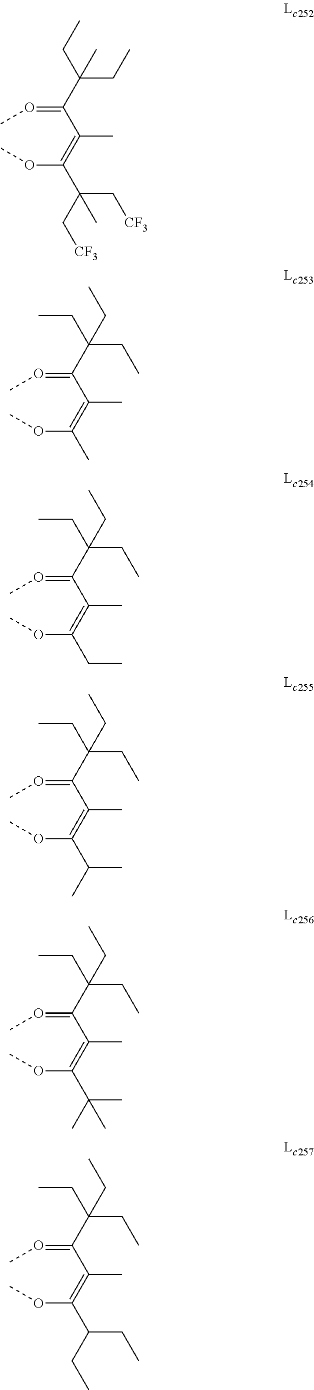

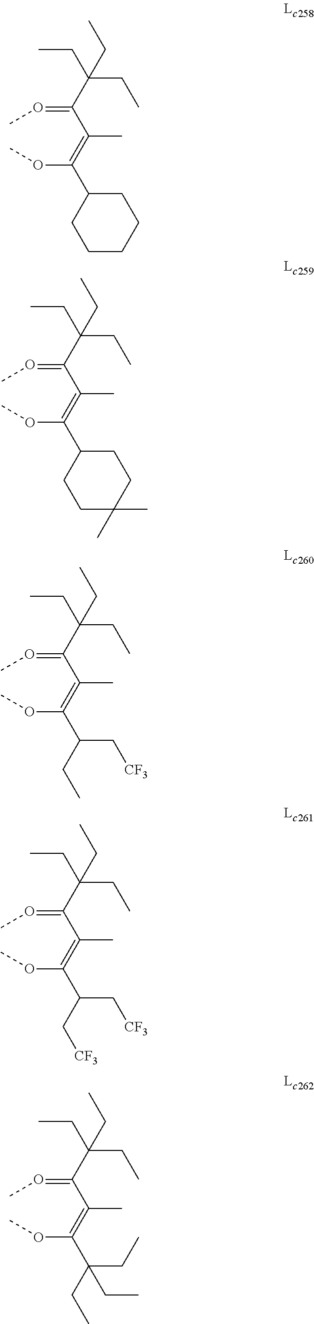

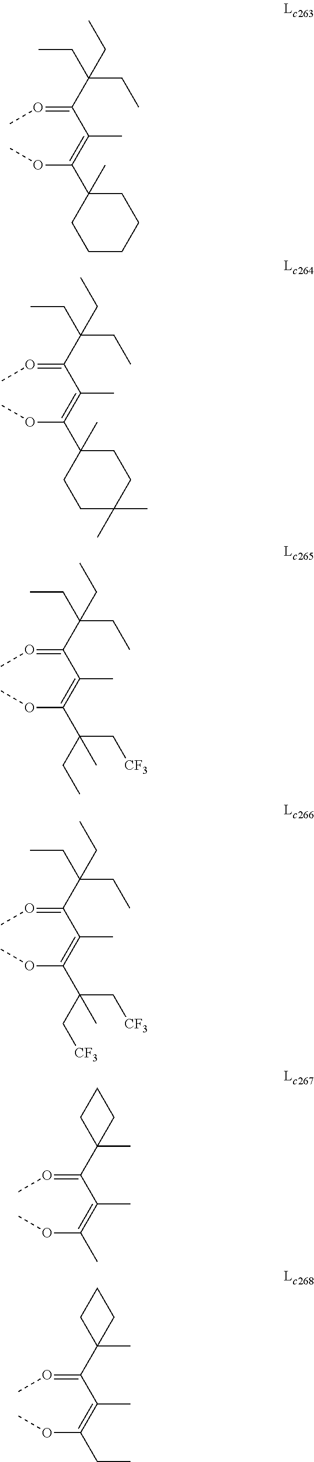

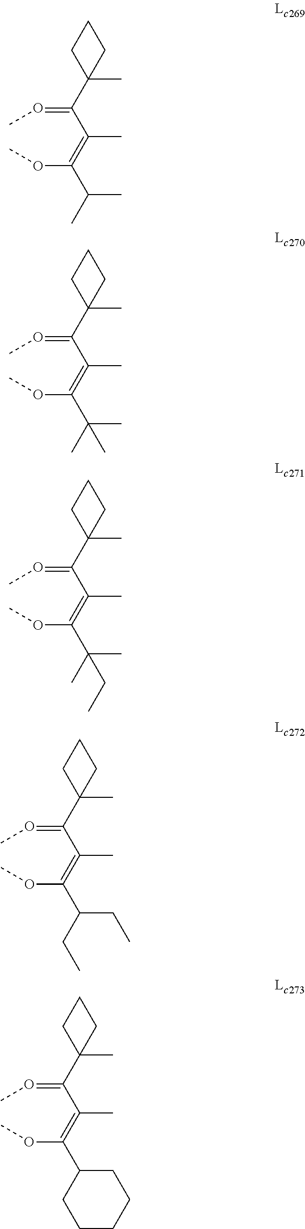

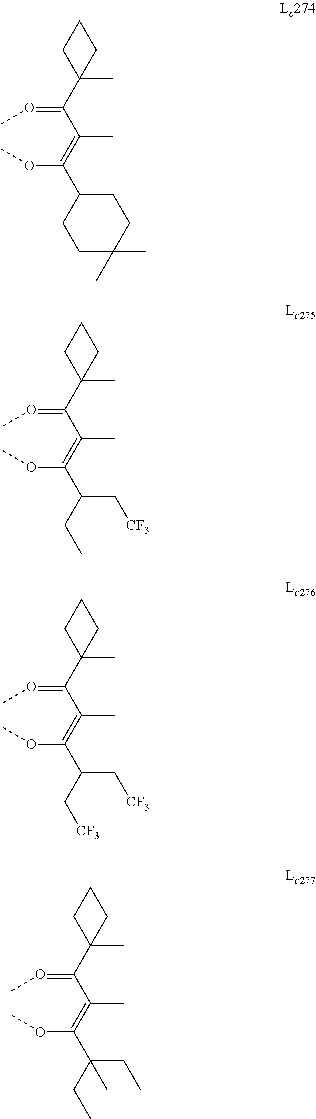

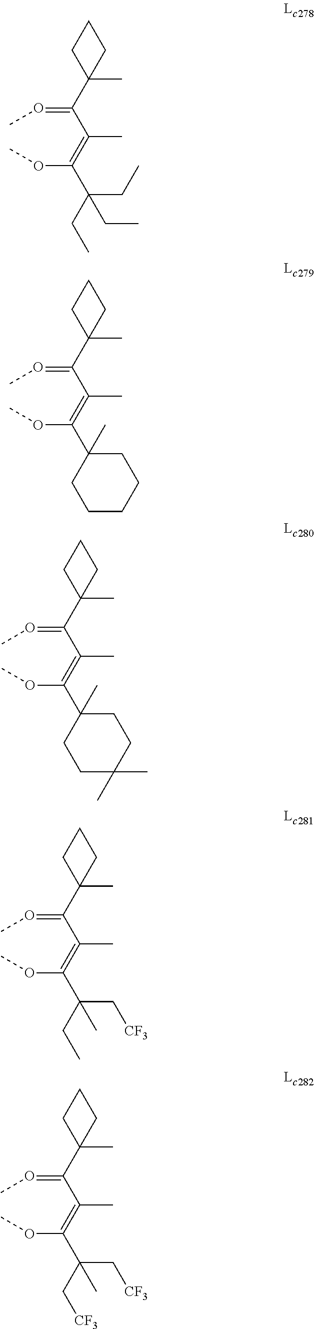

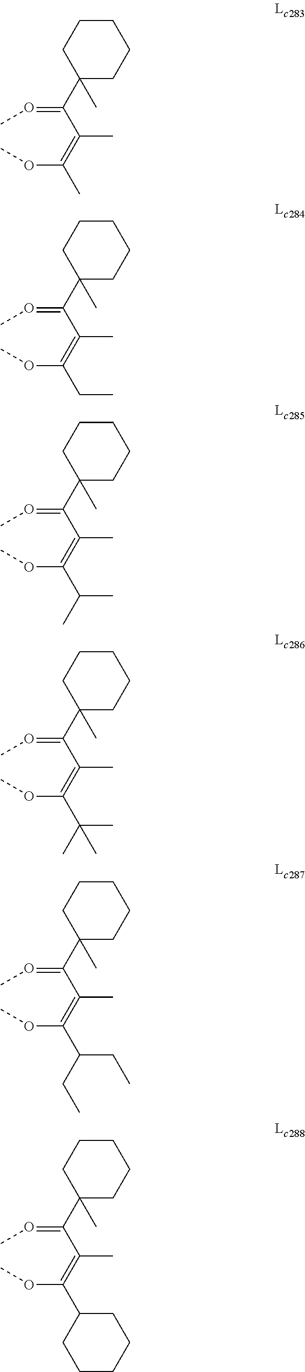

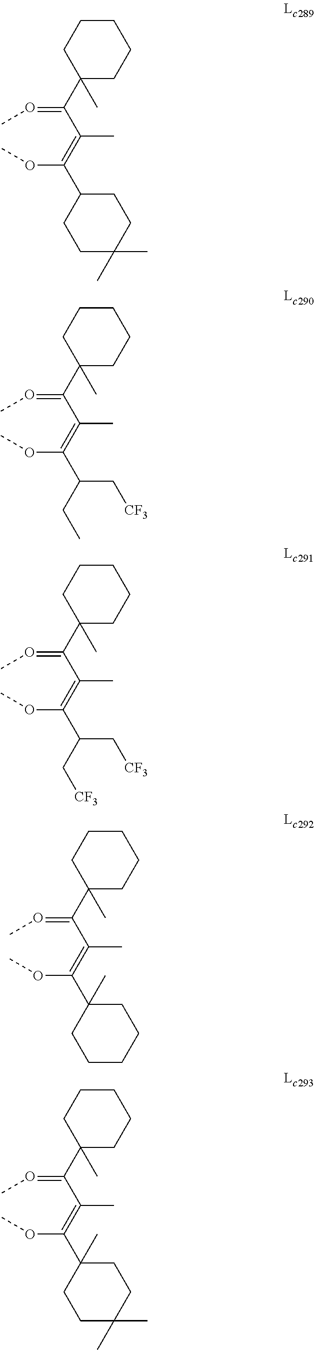

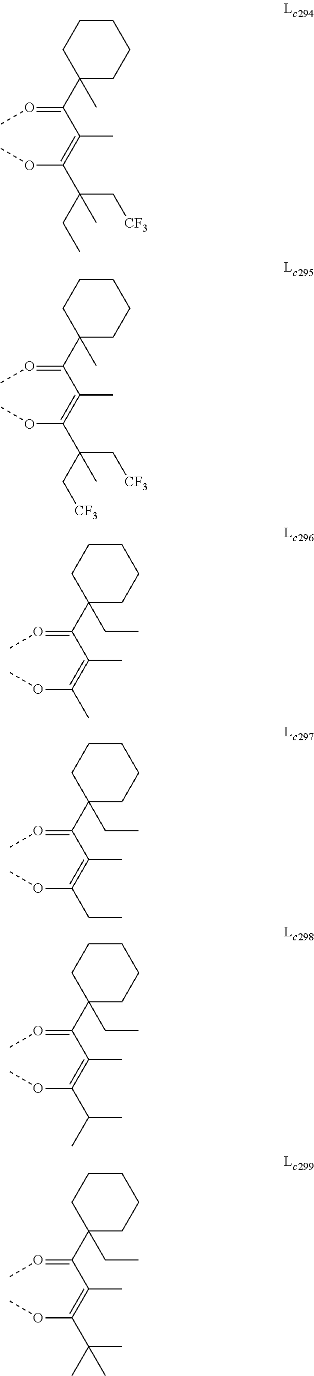

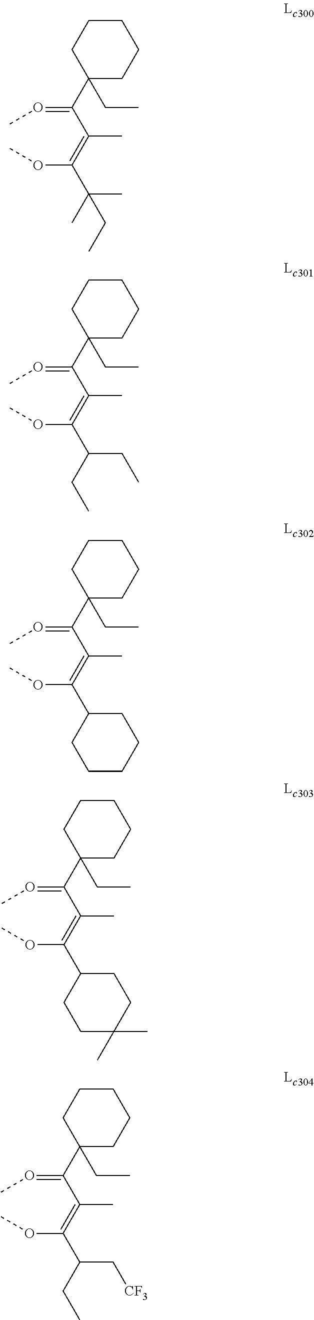

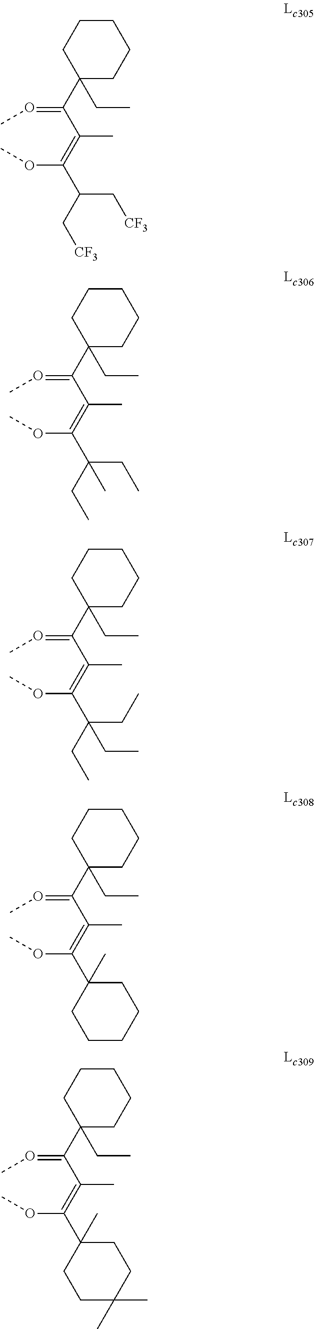

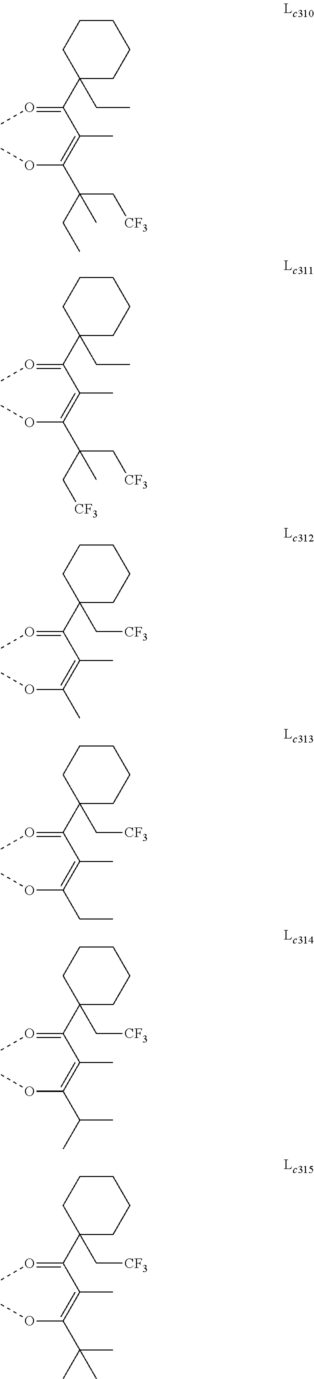

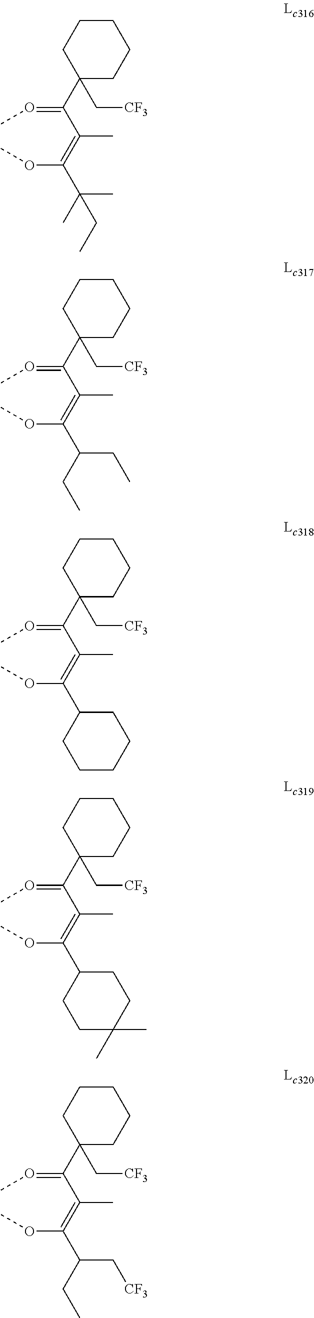

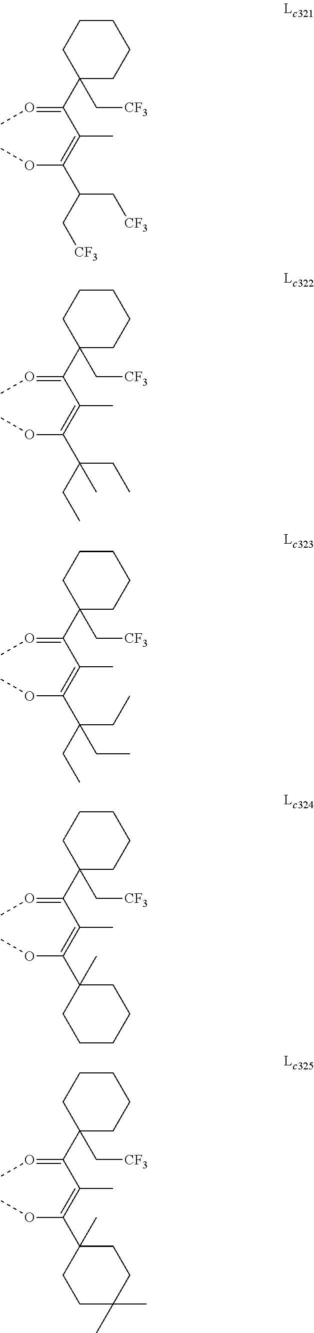

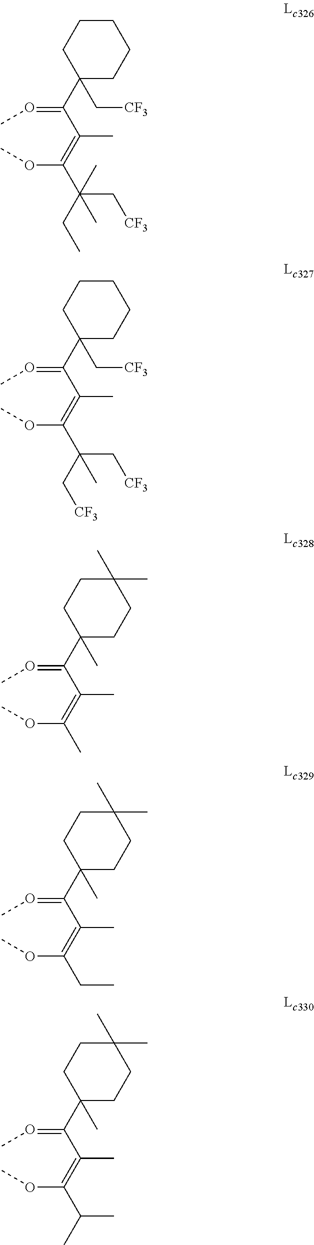

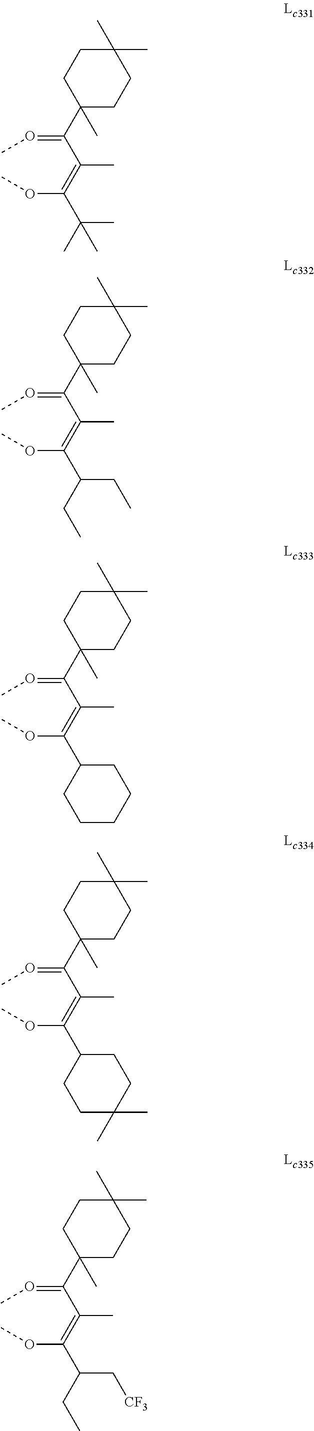

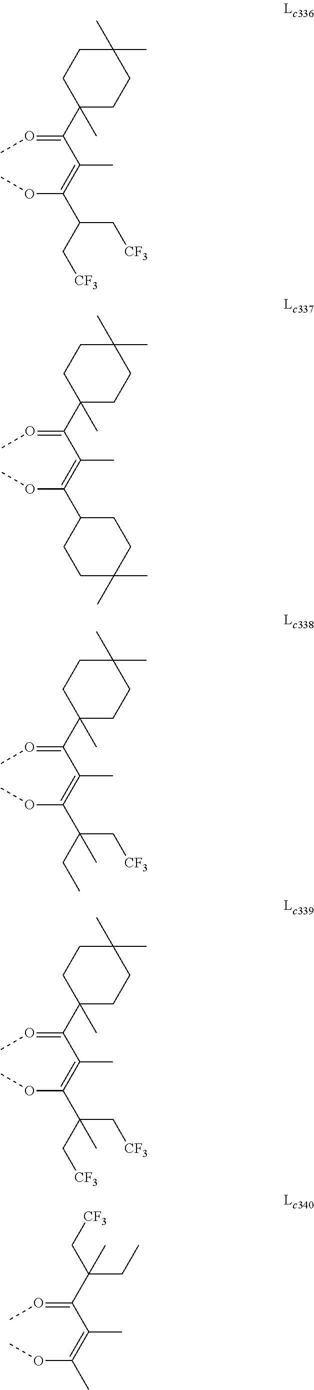

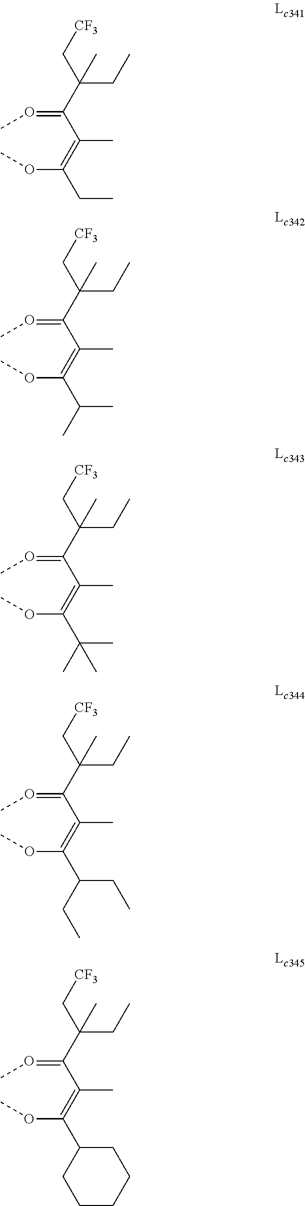

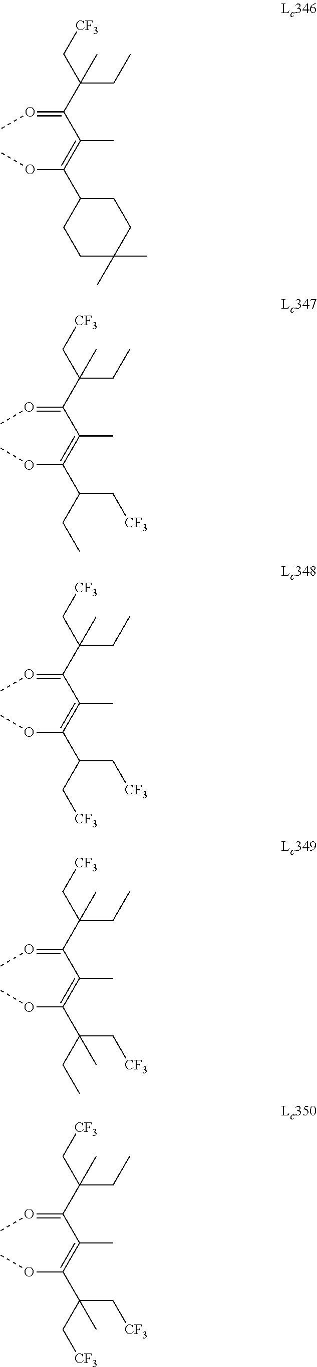

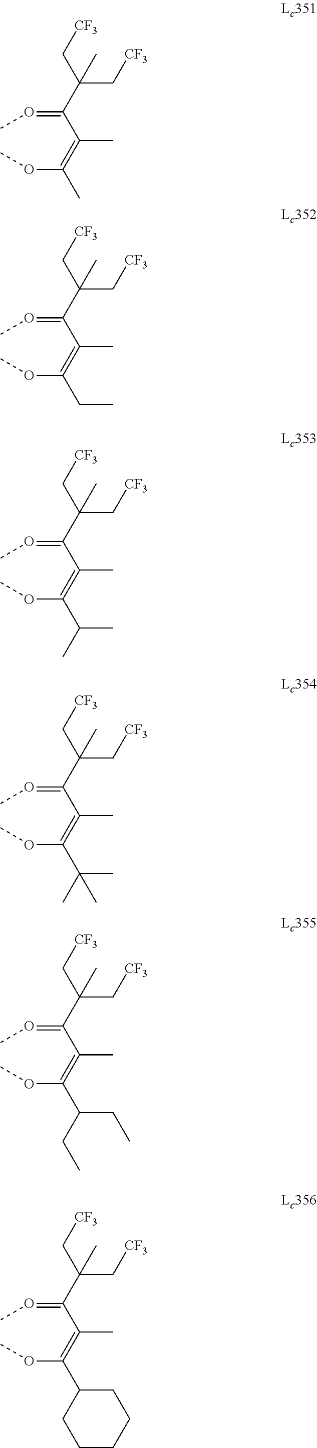

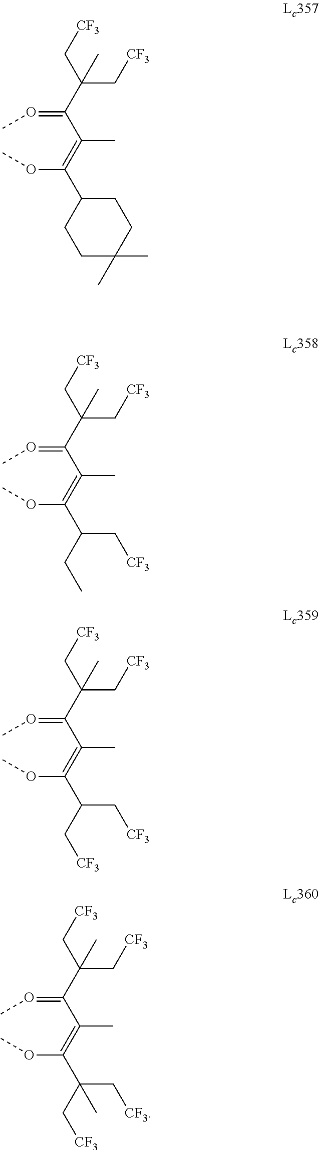

20. The metal complex according to claim 18, wherein the metal complex has a structure of Ir(La)₂Lb, wherein the two Lₐ are identical or different, Lₐ is selected from the group consisting of Lₐ₁₋₁ to Lₐ₁₋₂₃₁, Lₐ₂₋₁ to Lₐ₂₋₁₆₁ and Lₐ₃₋₁ to Lₐ₃₋₁₃₀, and L_{b} is selected from the group consisting of L_{b1-1} to L_{b1-355}, L_{b2-1} to L_{b2-261} and L_{b3-1} to L_{b3-650};
preferably, the metal complex is selected from the group consisting of Metal Complex 1 to Metal Complex 2128, wherein Metal Complex 1 to Metal Complex 2128 have the structure of Ir(Lₐ)₂L_{b}, wherein the two Lₐ are identical and Lₐ and L_{b} correspond to structures shown in the following table, respectively:

| Metal | | | Metal | | |
|---|---|---|---|---|---|
| Complex | | | Complex | | |
| No. | Lₐ | L_{b} | No. | Lₐ | L_{b} |
| 1 | Lₐ₁₋₁ | L_{b1-1} | 2 | Lₐ₁₋₁ | L_{b1-2} |
| 3 | Lₐ₁₋₁ | L_{b1-3} | 4 | Lₐ₁₋₁ | L_{b1-4} |
| 5 | Lₐ₁₋₁ | L_{b1-5} | 6 | Lₐ₁₋₁ | L_{b1-6} |
| 7 | Lₐ₁₋₁ | L_{b1-7} | 8 | Lₐ₁₋₁ | L_{b1-8} |
| 9 | Lₐ₁₋₁ | L_{b1-9} | 10 | Lₐ₁₋₁ | L_{b1-10} |
| 11 | Lₐ₁₋₁ | L_{b1-11} | 12 | Lₐ₁₋₁ | L_{b1-12} |
| 13 | Lₐ₁₋₁ | L_{b1-13} | 14 | Lₐ₁₋₁ | L_{b1-14} |
| 15 | Lₐ₁₋₁ | L_{b1-15} | 16 | Lₐ₁₋₁ | L_{b1-16} |
| 17 | Lₐ₁₋₁ | L_{b1-17} | 18 | Lₐ₁₋₁ | L_{b1-18} |
| 19 | Lₐ₁₋₁ | L_{b1-19} | 20 | Lₐ₁₋₁ | L_{b1-20} |
| 21 | Lₐ₁₋₁ | L_{b1-21} | 22 | Lₐ₁₋₁ | L_{b1-22} |
| 23 | Lₐ₁₋₁ | L_{b1-23} | 24 | Lₐ₁₋₁ | L_{b1-24} |
| 25 | Lₐ₁₋₁ | L_{b1-25} | 26 | Lₐ₁₋₁ | L_{b1-26} |
| 27 | Lₐ₁₋₁ | L_{b1-27} | 28 | Lₐ₁₋₁ | L_{b1-28} |
| 29 | Lₐ₁₋₁ | L_{b1-29} | 30 | Lₐ₁₋₁ | L_{b1-30} |
| 31 | Lₐ₁₋₁ | L_{b1-45} | 32 | Lₐ₁₋₁ | L_{b1-46} |
| 33 | Lₐ₁₋₁ | L_{b1-47} | 34 | Lₐ₁₋₁ | L_{b1-48} |
| 35 | Lₐ₁₋₁ | L_{b1-49} | 36 | Lₐ₁₋₁ | L_{b1-50} |
| 37 | Lₐ₁₋₁ | L_{b1-51} | 38 | Lₐ₁₋₁ | L_{b1-52} |
| 39 | Lₐ₁₋₁ | L_{b1-53} | 40 | Lₐ₁₋₁ | L_{b1-54} |
| 41 | Lₐ₁₋₁ | L_{b1-55} | 42 | Lₐ₁₋₁ | L_{b1-56} |
| 43 | Lₐ₁₋₁ | L_{b1-57} | 44 | Lₐ₁₋₁ | L_{b1-58} |
| 45 | Lₐ₁₋₁ | L_{b1-59} | 46 | Lₐ₁₋₁ | L_{b1-60} |
| 47 | Lₐ₁₋₁ | L_{b1-61} | 48 | Lₐ₁₋₁ | L_{b1-62} |
| 49 | Lₐ₁₋₁ | L_{b1-63} | 50 | Lₐ₁₋₁ | L_{b1-64} |
| 51 | Lₐ₁₋₁ | L_{b1-65} | 52 | Lₐ₁₋₁ | L_{b1-66} |
| 53 | Lₐ₁₋₁ | L_{b1-67} | 54 | Lₐ₁₋₁ | L_{b1-68} |
| 55 | Lₐ₁₋₁ | L_{b1-69} | 56 | Lₐ₁₋₁ | L_{b1-70} |
| 57 | Lₐ₁₋₁ | L_{b1-71} | 58 | Lₐ₁₋₁ | L_{b1-72} |
| 59 | Lₐ₁₋₁ | L_{b1-73} | 60 | Lₐ₁₋₁ | L_{b1-74} |
| 61 | Lₐ₁₋₁ | L_{b1-75} | 62 | Lₐ₁₋₁ | L_{b1-76} |
| 63 | Lₐ₁₋₁ | L_{b1-77} | 64 | Lₐ₁₋₁ | L_{b1-78} |
| 65 | Lₐ₁₋₁ | L_{b1-79} | 66 | Lₐ₁₋₁ | L_{b1-80} |
| 67 | Lₐ₁₋₁ | L_{b1-81} | 68 | Lₐ₁₋₁ | L_{b1-82} |
| 69 | Lₐ₁₋₁ | L_{b1-83} | 70 | Lₐ₁₋₁ | L_{b1-84} |
| 71 | Lₐ₁₋₁ | L_{b1-85} | 72 | Lₐ₁₋₁ | L_{b1-86} |
| 73 | Lₐ₁₋₁ | L_{b1-87} | 74 | Lₐ₁₋₁ | L_{b1-88} |
| 75 | Lₐ₁₋₁ | L_{b1-89} | 76 | Lₐ₁₋₁ | L_{b1-90} |
| 77 | Lₐ₁₋₁ | L_{b1-91} | 78 | Lₐ₁₋₁ | L_{b1-92} |
| 79 | Lₐ₁₋₁ | L_{b1-120} | 80 | Lₐ₁₋₁ | L_{b1-121} |
| 81 | Lₐ₁₋₁ | L_{b1-122} | 82 | Lₐ₁₋₁ | L_{b1-123} |
| 83 | Lₐ₁₋₁ | L_{b1-124} | 84 | Lₐ₁₋₁ | L_{b1-125} |
| 85 | Lₐ₁₋₁ | L_{b1-126} | 86 | Lₐ₁₋₁ | L_{b1-127} |
| 87 | Lₐ₁₋₁ | L_{b1-159} | 88 | Lₐ₁₋₁ | L_{b1-160} |
| 89 | Lₐ₁₋₁ | L_{b1-161} | 90 | Lₐ₁₋₁ | L_{b1-162} |
| 91 | Lₐ₁₋₁ | L_{b1-282} | 92 | Lₐ₁₋₁ | L_{b1-283} |
| 93 | Lₐ₁₋₁ | L_{b1-284} | 94 | Lₐ₁₋₁ | L_{b1-285} |
| 95 | Lₐ₁₋₁ | L_{b1-286} | 96 | Lₐ₁₋₁ | L_{b1-287} |
| 97 | Lₐ₁₋₁ | L_{b1-288} | 98 | Lₐ₁₋₁ | L_{b1-289} |
| 99 | Lₐ₁₋₁ | L_{b1-290} | 100 | Lₐ₁₋₁ | L_{b1-291} |
| 101 | Lₐ₁₋₁ | L_{b1-292} | 102 | Lₐ₁₋₁ | L_{b1-293} |
| 103 | Lₐ₁₋₁ | L_{b1-294} | 104 | Lₐ₁₋₁ | L_{b1-295} |
| 105 | Lₐ₁₋₁ | L_{b1-296} | 106 | Lₐ₁₋₁ | L_{b1-297} |
| 107 | Lₐ₁₋₁ | L_{b1-298} | 108 | Lₐ₁₋₁ | L_{b1-299} |
| 109 | Lₐ₁₋₁ | L_{b1-300} | 110 | Lₐ₁₋₁ | L_{b1-301} |
| 111 | Lₐ₁₋₁ | L_{b1-302} | 112 | Lₐ₁₋₁ | L_{b1-303} |
| 113 | Lₐ₁₋₁ | L_{b1-304} | 114 | Lₐ₁₋₁ | L_{b1-305} |
| 115 | Lₐ₁₋₁ | L_{b1-306} | 116 | Lₐ₁₋₁ | L_{b1-307} |
| 117 | Lₐ₁₋₁ | L_{b1-308} | 118 | Lₐ₁₋₁ | L_{b1-309} |
| 119 | Lₐ₁₋₁ | L_{b1-310} | 120 | Lₐ₁₋₁ | L_{b1-311} |
| 121 | Lₐ₁₋₁ | L_{b1-312} | 122 | Lₐ₁₋₁ | L_{b1-313} |
| 123 | Lₐ₁₋₁ | L_{b1-314} | 124 | Lₐ₁₋₁ | L_{b1-315} |
| 125 | Lₐ₁₋₁ | L_{b1-316} | 126 | Lₐ₁₋₁ | L_{b1-317} |
| 127 | Lₐ₁₋₁ | L_{b1-318} | 128 | Lₐ₁₋₁ | L_{b1-319} |
| 129 | Lₐ₁₋₁ | L_{b1-320} | 130 | Lₐ₁₋₁ | L_{b1-321} |
| 131 | Lₐ₁₋₁ | L_{b1-322} | 132 | Lₐ₁₋₁ | L_{b1-323} |
| 133 | Lₐ₁₋₁ | L_{b1-342} | 134 | Lₐ₁₋₁ | L_{b1-343} |
| 135 | Lₐ₁₋₁ | L_{b1-344} | 136 | Lₐ₁₋₁ | L_{b1-344} |
| 137 | Lₐ₁₋₁ | L_{b1-346} | 138 | Lₐ₁₋₁ | L_{b1-345} |
| 139 | Lₐ₁₋₁ | L_{b1-348} | 140 | Lₐ₁₋₁ | L_{b1-346} |
| 141 | Lₐ₁₋₁ | L_{b1-350} | 142 | Lₐ₁₋₁ | L_{b1-347} |
| 143 | Lₐ₁₋₁ | L_{b1-352} | 144 | Lₐ₁₋₁ | L_{b1-348} |
| 145 | Lₐ₁₋₁ | L_{b1-354} | 146 | Lₐ₁₋₁ | L_{b1-349} |
| 147 | Lₐ₁₋₁ | L_{b2-1} | 148 | Lₐ₁₋₁ | L_{b2-2} |
| 149 | Lₐ₁₋₁ | L_{b2-3} | 150 | Lₐ₁₋₁ | L_{b2-4} |
| 151 | Lₐ₁₋₁ | L_{b2-5} | 152 | Lₐ₁₋₁ | L_{b2-6} |
| 153 | Lₐ₁₋₁ | L_{b2-7} | 154 | Lₐ₁₋₁ | L_{b2-8} |
| 155 | Lₐ₁₋₁ | L_{b2-9} | 156 | Lₐ₁₋₁ | L_{b2-10} |
| 157 | Lₐ₁₋₁ | L_{b2-11} | 158 | Lₐ₁₋₁ | L_{b2-12} |
| 159 | Lₐ₁₋₁ | L_{b2-22} | 160 | Lₐ₁₋₁ | L_{b2-23} |
| 161 | Lₐ₁₋₁ | L_{b2-24} | 162 | Lₐ₁₋₁ | L_{b2-25} |
| 163 | Lₐ₁₋₁ | L_{b2-26} | 164 | Lₐ₁₋₁ | L_{b2-27} |
| 165 | Lₐ₁₋₁ | L_{b2-28} | 166 | Lₐ₁₋₁ | L_{b2-29} |
| 167 | Lₐ₁₋₁ | L_{b2-30} | 168 | Lₐ₁₋₁ | L_{b2-31} |
| 169 | Lₐ₁₋₁ | L_{b2-32} | 170 | Lₐ₁₋₁ | L_{b2-33} |
| 171 | Lₐ₁₋₁ | L_{b2-34} | 172 | Lₐ₁₋₁ | L_{b2-35} |
| 173 | Lₐ₁₋₁ | L_{b2-36} | 174 | Lₐ₁₋₁ | L_{b2-37} |
| 175 | Lₐ₁₋₁ | L_{b2-49} | 176 | Lₐ₁₋₁ | L_{b2-50} |
| 177 | Lₐ₁₋₁ | L_{b2-51} | 178 | Lₐ₁₋₁ | L_{b2-52} |
| 179 | Lₐ₁₋₁ | L_{b2-53} | 180 | Lₐ₁₋₁ | L_{b2-54} |
| 181 | Lₐ₁₋₁ | L_{b2-55} | 182 | Lₐ₁₋₁ | L_{b2-56} |
| 183 | Lₐ₁₋₁ | L_{b2-57} | 184 | Lₐ₁₋₁ | L_{b2-58} |
| 185 | Lₐ₁₋₁ | L_{b2-59} | 186 | Lₐ₁₋₁ | L_{b2-60} |
| 187 | Lₐ₁₋₁ | L_{b2-61} | 188 | Lₐ₁₋₁ | L_{b2-62} |
| 189 | Lₐ₁₋₁ | L_{b2-63} | 190 | Lₐ₁₋₁ | L_{b2-64} |
| 191 | Lₐ₁₋₁ | L_{b2-65} | 192 | Lₐ₁₋₁ | L_{b2-66} |
| 193 | Lₐ₁₋₁ | L_{b2-67} | 194 | Lₐ₁₋₁ | L_{b2-68} |
| 195 | Lₐ₁₋₁ | L_{b2-69} | 196 | Lₐ₁₋₁ | L_{b2-70} |
| 197 | Lₐ₁₋₁ | L_{b2-94} | 198 | Lₐ₁₋₁ | L_{b2-95} |
| 199 | Lₐ₁₋₁ | L_{b2-164} | 200 | Lₐ₁₋₁ | L_{b2-165} |
| 201 | Lₐ₁₋₁ | L_{b2-166} | 202 | Lₐ₁₋₁ | L_{b2-167} |
| 203 | Lₐ₁₋₁ | L_{b2-168} | 204 | Lₐ₁₋₁ | L_{b2-169} |
| 205 | Lₐ₁₋₁ | L_{b2-170} | 206 | Lₐ₁₋₁ | L_{b2-171} |
| 207 | Lₐ₁₋₁ | L_{b2-172} | 208 | Lₐ₁₋₁ | L_{b2-173} |
| 209 | Lₐ₁₋₁ | L_{b2-174} | 210 | Lₐ₁₋₁ | L_{b2-175} |
| 211 | Lₐ₁₋₁ | L_{b2-176} | 212 | Lₐ₁₋₁ | L_{b2-177} |
| 213 | Lₐ₁₋₁ | L_{b2-178} | 214 | Lₐ₁₋₁ | L_{b2-179} |
| 215 | Lₐ₁₋₁ | L_{b2-180} | 216 | Lₐ₁₋₁ | L_{b2-181} |
| 217 | Lₐ₁₋₁ | L_{b2-182} | 218 | Lₐ₁₋₁ | L_{b2-183} |
| 219 | Lₐ₁₋₁ | L_{b2-184} | 220 | Lₐ₁₋₁ | L_{b2-185} |
| 221 | Lₐ₁₋₁ | L_{b2-186} | 222 | Lₐ₁₋₁ | L_{b2-187} |
| 223 | Lₐ₁₋₁ | L_{b2-188} | 224 | Lₐ₁₋₁ | L_{b2-189} |
| 225 | Lₐ₁₋₁ | L_{b2-190} | 226 | Lₐ₁₋₁ | L_{b2-191} |
| 227 | Lₐ₁₋₁ | L_{b2-192} | 228 | Lₐ₁₋₁ | L_{b2-193} |
| 229 | Lₐ₁₋₁ | L_{b2-194} | 230 | Lₐ₁₋₁ | L_{b2-195} |
| 231 | Lₐ₁₋₁ | L_{b2-196} | 232 | Lₐ₁₋₁ | L_{b2-197} |
| 233 | Lₐ₁₋₁ | L_{b2-198} | 234 | Lₐ₁₋₁ | L_{b2-199} |
| 235 | Lₐ₁₋₁ | L_{b2-200} | 236 | Lₐ₁₋₁ | L_{b2-201} |
| 237 | Lₐ₁₋₁ | L_{b2-202} | 238 | Lₐ₁₋₁ | L_{b2-203} |
| 239 | Lₐ₁₋₁ | L_{b2-204} | 240 | Lₐ₁₋₁ | L_{b2-205} |
| 241 | Lₐ₁₋₁ | L_{b2-214} | 242 | Lₐ₁₋₁ | L_{b2-215} |
| 243 | Lₐ₁₋₁ | L_{b2-216} | 244 | Lₐ₁₋₁ | L_{b2-217} |
| 245 | Lₐ₁₋₁ | L_{b2-218} | 246 | Lₐ₁₋₁ | L_{b2-219} |
| 247 | Lₐ₁₋₁ | L_{b2-232} | 248 | Lₐ₁₋₁ | L_{b2-233} |
| 249 | Lₐ₁₋₁ | L_{b2-234} | 250 | Lₐ₁₋₁ | L_{b2-235} |
| 251 | Lₐ₁₋₁ | L_{b2-236} | 252 | Lₐ₁₋₁ | L_{b2-237} |
| 253 | Lₐ₁₋₁ | L_{b2-238} | 254 | Lₐ₁₋₁ | L_{b2-239} |
| 255 | Lₐ₁₋₁ | L_{b2-240} | 256 | Lₐ₁₋₁ | L_{b2-241} |
| 257 | Lₐ₁₋₁ | L_{b2-242} | 258 | Lₐ₁₋₁ | L_{b2-243} |
| 259 | Lₐ₁₋₁ | L_{b3-1} | 260 | Lₐ₁₋₁ | L_{b3-2} |
| 261 | Lₐ₁₋₁ | L_{b3-3} | 262 | Lₐ₁₋₁ | L_{b3-4} |
| 263 | Lₐ₁₋₁ | L_{b3-38} | 264 | Lₐ₁₋₁ | L_{b3-39} |
| 265 | Lₐ₁₋₁ | L_{b3-57} | 266 | Lₐ₁₋₁ | L_{b3-58} |
| 267 | Lₐ₁₋₁ | L_{b3-59} | 268 | Lₐ₁₋₁ | L_{b3-60} |
| 269 | Lₐ₁₋₁ | L_{b3-59} | 270 | Lₐ₁₋₁ | L_{b3-60} |
| 271 | Lₐ₁₋₁ | L_{b3-60} | 272 | Lₐ₁₋₁ | L_{b3-61} |
| 273 | Lₐ₁₋₁ | L_{b3-61} | 274 | Lₐ₁₋₁ | L_{b3-62} |
| 275 | Lₐ₁₋₁ | L_{b3-66} | 276 | Lₐ₁₋₁ | L_{b3-67} |
| 277 | Lₐ₁₋₁ | L_{b3-68} | 278 | Lₐ₁₋₁ | L_{b3-69} |
| 279 | Lₐ₁₋₁ | L_{b3-80} | 280 | Lₐ₁₋₁ | L_{b3-81} |
| 281 | Lₐ₁₋₁ | L_{b3-316} | 282 | Lₐ₁₋₁ | L_{b3-317} |
| 283 | Lₐ₁₋₁ | L_{b3-337} | 284 | Lₐ₁₋₁ | L_{b3-338} |
| 285 | Lₐ₁₋₁ | L_{b3-380} | 286 | Lₐ₁₋₁ | L_{b3-381} |
| 287 | Lₐ₁₋₁ | L_{b3-384} | 288 | Lₐ₁₋₁ | L_{b3-386} |
| 289 | Lₐ₁₋₁ | L_{b3-390} | 290 | Lₐ₁₋₁ | L_{b3-410} |
| 291 | Lₐ₁₋₁ | L_{b3-422} | 292 | Lₐ₁₋₁ | L_{b3-433} |
| 293 | Lₐ₁₋₁ | L_{b3-444} | 294 | Lₐ₁₋₁ | L_{b3-450} |
| 295 | Lₐ₁₋₁ | L_{b3-451} | 296 | Lₐ₁₋₁ | L_{b3-470} |
| 297 | Lₐ₁₋₁ | L_{b3-479} | 298 | Lₐ₁₋₁ | L_{b3-480} |
| 299 | Lₐ₁₋₁ | L_{b3-484} | 300 | Lₐ₁₋₁ | L_{b3-490} |
| 301 | Lₐ₁₋₁ | L_{b3-504} | 302 | Lₐ₁₋₁ | L_{b3-508} |
| 303 | Lₐ₁₋₁ | L_{b3-509} | 304 | Lₐ₁₋₁ | L_{b3-510} |
| 305 | Lₐ₁₋₁ | L_{b3-533} | 306 | Lₐ₁₋₁ | L_{b3-550} |
| 307 | Lₐ₁₋₁ | L_{b3-562} | 308 | Lₐ₁₋₁ | L_{b3-574} |
| 309 | Lₐ₁₋₁ | L_{b3-578} | 310 | Lₐ₁₋₁ | L_{b3-584} |
| 311 | Lₐ₁₋₈ | L_{b1-1} | 312 | Lₐ₁₋₈ | L_{b1-2} |
| 313 | Lₐ₁₋₈ | L_{b1-3} | 314 | Lₐ₁₋₈ | L_{b1-4} |
| 315 | Lₐ₁₋₈ | L_{b1-5} | 316 | Lₐ₁₋₈ | L_{b1-6} |
| 317 | Lₐ₁₋₈ | L_{b1-7} | 318 | Lₐ₁₋₈ | L_{b1-8} |
| 319 | Lₐ₁₋₈ | L_{b1-9} | 320 | Lₐ₁₋₈ | L_{b1-10} |
| 321 | Lₐ₁₋₈ | L_{b1-11} | 322 | Lₐ₁₋₈ | L_{b1-12} |
| 323 | Lₐ₁₋₈ | L_{b1-13} | 324 | Lₐ₁₋₈ | L_{b1-14} |
| 325 | Lₐ₁₋₈ | L_{b1-15} | 326 | Lₐ₁₋₈ | L_{b1-16} |
| 327 | Lₐ₁₋₈ | L_{b1-17} | 328 | Lₐ₁₋₈ | L_{b1-18} |
| 329 | Lₐ₁₋₈ | L_{b1-19} | 330 | Lₐ₁₋₈ | L_{b1-20} |
| 331 | Lₐ₁₋₈ | L_{b1-21} | 332 | Lₐ₁₋₈ | L_{b1-22} |
| 333 | Lₐ₁₋₈ | L_{b1-23} | 334 | Lₐ₁₋₈ | L_{b1-24} |
| 335 | Lₐ₁₋₈ | L_{b1-25} | 336 | Lₐ₁₋₈ | L_{b1-26} |
| 337 | Lₐ₁₋₈ | L_{b1-27} | 338 | Lₐ₁₋₈ | L_{b1-28} |
| 339 | Lₐ₁₋₈ | L_{b1-29} | 340 | Lₐ₁₋₈ | L_{b1-30} |
| 341 | Lₐ₁₋₈ | L_{b1-45} | 342 | Lₐ₁₋₈ | L_{b1-46} |
| 343 | Lₐ₁₋₈ | L_{b1-47} | 344 | Lₐ₁₋₈ | L_{b1-48} |
| 345 | Lₐ₁₋₈ | L_{b1-49} | 346 | Lₐ₁₋₈ | L_{b1-50} |
| 347 | Lₐ₁₋₈ | L_{b1-51} | 348 | Lₐ₁₋₈ | L_{b1-52} |
| 349 | Lₐ₁₋₈ | L_{b1-53} | 350 | Lₐ₁₋₈ | L_{b1-54} |
| 351 | Lₐ₁₋₈ | L_{b1-55} | 352 | Lₐ₁₋₈ | L_{b1-56} |
| 353 | Lₐ₁₋₈ | L_{b1-57} | 354 | Lₐ₁₋₈ | L_{b1-58} |
| 355 | Lₐ₁₋₈ | L_{b1-59} | 356 | Lₐ₁₋₈ | L_{b1-60} |
| 357 | Lₐ₁₋₈ | L_{b1-61} | 358 | Lₐ₁₋₈ | L_{b1-62} |
| 359 | Lₐ₁₋₈ | L_{b1-63} | 360 | Lₐ₁₋₈ | L_{b1-64} |
| 361 | Lₐ₁₋₈ | L_{b1-65} | 362 | Lₐ₁₋₈ | L_{b1-66} |
| 363 | Lₐ₁₋₈ | L_{b1-67} | 364 | Lₐ₁₋₈ | L_{b1-68} |
| 365 | Lₐ₁₋₈ | L_{b1-69} | 366 | Lₐ₁₋₈ | L_{b1-70} |
| 367 | Lₐ₁₋₈ | L_{b1-71} | 368 | Lₐ₁₋₈ | L_{b1-72} |
| 369 | Lₐ₁₋₈ | L_{b1-73} | 370 | Lₐ₁₋₈ | L_{b1-74} |
| 371 | Lₐ₁₋₈ | L_{b1-75} | 372 | Lₐ₁₋₈ | L_{b1-76} |
| 373 | Lₐ₁₋₈ | L_{b1-77} | 374 | Lₐ₁₋₈ | L_{b1-78} |
| 375 | Lₐ₁₋₈ | L_{b1-79} | 376 | Lₐ₁₋₈ | L_{b1-80} |
| 377 | Lₐ₁₋₈ | L_{b1-81} | 378 | Lₐ₁₋₈ | L_{b1-82} |
| 379 | Lₐ₁₋₈ | L_{b1-83} | 380 | Lₐ₁₋₈ | L_{b1-84} |
| 381 | Lₐ₁₋₈ | L_{b1-85} | 382 | Lₐ₁₋₈ | L_{b1-86} |
| 383 | Lₐ₁₋₈ | L_{b1-87} | 384 | Lₐ₁₋₈ | L_{b1-88} |
| 385 | Lₐ₁₋₈ | L_{b1-89} | 386 | Lₐ₁₋₈ | L_{b1-90} |
| 387 | Lₐ₁₋₈ | L_{b1-91} | 388 | Lₐ₁₋₈ | L_{b1-92} |
| 389 | Lₐ₁₋₈ | L_{b1-120} | 390 | Lₐ₁₋₈ | L_{b1-121} |
| 391 | Lₐ₁₋₈ | L_{b1-122} | 392 | Lₐ₁₋₈ | L_{b1-123} |
| 393 | Lₐ₁₋₈ | L_{b1-124} | 394 | Lₐ₁₋₈ | L_{b1-125} |
| 395 | Lₐ₁₋₈ | L_{b1-126} | 396 | Lₐ₁₋₈ | L_{b1-127} |
| 397 | Lₐ₁₋₈ | L_{b1-159} | 398 | Lₐ₁₋₈ | L_{b1-160} |
| 399 | Lₐ₁₋₈ | L_{b1-161} | 400 | Lₐ₁₋₈ | L_{b1-162} |
| 401 | Lₐ₁₋₈ | L_{b1-282} | 402 | Lₐ₁₋₈ | L_{b1-283} |
| 403 | Lₐ₁₋₈ | L_{b1-284} | 404 | Lₐ₁₋₈ | L_{b1-285} |
| 405 | Lₐ₁₋₈ | L_{b1-286} | 406 | Lₐ₁₋₈ | L_{b1-287} |
| 407 | Lₐ₁₋₈ | L_{b1-288} | 408 | Lₐ₁₋₈ | L_{b1-289} |
| 409 | Lₐ₁₋₈ | L_{b1-290} | 410 | Lₐ₁₋₈ | L_{b1-291} |
| 411 | Lₐ₁₋₈ | L_{b1-292} | 412 | Lₐ₁₋₈ | L_{b1-293} |
| 413 | Lₐ₁₋₈ | L_{b1-294} | 414 | Lₐ₁₋₈ | L_{b1-295} |
| 415 | Lₐ₁₋₈ | L_{b1-296} | 416 | Lₐ₁₋₈ | L_{b1-297} |
| 417 | Lₐ₁₋₈ | L_{b1-298} | 418 | Lₐ₁₋₈ | L_{b1-299} |
| 419 | Lₐ₁₋₈ | L_{b1-300} | 420 | Lₐ₁₋₈ | L_{b1-301} |
| 421 | Lₐ₁₋₈ | L_{b1-302} | 422 | Lₐ₁₋₈ | L_{b1-303} |
| 423 | Lₐ₁₋₈ | L_{b1-304} | 424 | Lₐ₁₋₈ | L_{b1-305} |
| 425 | Lₐ₁₋₈ | L_{b1-306} | 426 | Lₐ₁₋₈ | L_{b1-307} |
| 427 | Lₐ₁₋₈ | L_{b1-308} | 428 | Lₐ₁₋₈ | L_{b1-309} |
| 429 | Lₐ₁₋₈ | L_{b1-310} | 430 | Lₐ₁₋₈ | L_{b1-311} |
| 431 | Lₐ₁₋₈ | L_{b1-312} | 432 | Lₐ₁₋₈ | L_{b1-313} |
| 433 | Lₐ₁₋₈ | L_{b1-314} | 434 | Lₐ₁₋₈ | L_{b1-315} |
| 435 | Lₐ₁₋₈ | L_{b1-316} | 436 | Lₐ₁₋₈ | L_{b1-317} |
| 437 | Lₐ₁₋₈ | L_{b1-318} | 438 | Lₐ₁₋₈ | L_{b1-319} |
| 439 | Lₐ₁₋₈ | L_{b1-320} | 440 | Lₐ₁₋₈ | L_{b1-321} |
| 441 | Lₐ₁₋₈ | L_{b1-322} | 442 | Lₐ₁₋₈ | L_{b1-323} |
| 443 | Lₐ₁₋₈ | L_{b1-342} | 444 | Lₐ₁₋₈ | L_{b1-343} |
| 445 | Lₐ₁₋₈ | L_{b1-344} | 446 | Lₐ₁₋₈ | L_{b1-344} |
| 447 | Lₐ₁₋₈ | L_{b1-346} | 448 | Lₐ₁₋₈ | L_{b1-345} |
| 449 | Lₐ₁₋₈ | L_{b1-348} | 450 | Lₐ₁₋₈ | L_{b1-346} |
| 451 | Lₐ₁₋₈ | L_{b1-350} | 452 | Lₐ₁₋₈ | L_{b1-347} |
| 453 | Lₐ₁₋₈ | L_{b1-352} | 454 | Lₐ₁₋₈ | L_{b1-348} |
| 455 | Lₐ₁₋₈ | L_{b1-354} | 456 | Lₐ₁₋₈ | L_{b1-349} |
| 457 | Lₐ₁₋₈ | L_{b2-1} | 458 | Lₐ₁₋₈ | L_{b2-2} |
| 459 | Lₐ₁₋₈ | L_{b2-3} | 460 | Lₐ₁₋₈ | L_{b2-4} |
| 461 | Lₐ₁₋₈ | L_{b2-5} | 462 | Lₐ₁₋₈ | L_{b2-6} |
| 463 | Lₐ₁₋₈ | L_{b2-7} | 464 | Lₐ₁₋₈ | L_{b2-8} |
| 465 | Lₐ₁₋₈ | L_{b2-9} | 466 | Lₐ₁₋₈ | L_{b2-10} |
| 467 | Lₐ₁₋₈ | L_{b2-11} | 468 | Lₐ₁₋₈ | L_{b2-12} |
| 469 | Lₐ₁₋₈ | L_{b2-22} | 470 | Lₐ₁₋₈ | L_{b2-23} |
| 471 | Lₐ₁₋₈ | L_{b2-24} | 472 | Lₐ₁₋₈ | L_{b2-25} |
| 473 | Lₐ₁₋₈ | L_{b2-26} | 474 | Lₐ₁₋₈ | L_{b2-27} |
| 475 | Lₐ₁₋₈ | L_{b2-28} | 476 | Lₐ₁₋₈ | L_{b2-29} |
| 477 | Lₐ₁₋₈ | L_{b2-30} | 478 | Lₐ₁₋₈ | L_{b2-31} |
| 479 | Lₐ₁₋₈ | L_{b2-32} | 480 | Lₐ₁₋₈ | L_{b2-33} |
| 481 | Lₐ₁₋₈ | L_{b2-34} | 482 | Lₐ₁₋₈ | L_{b2-35} |
| 483 | Lₐ₁₋₈ | L_{b2-36} | 484 | Lₐ₁₋₈ | L_{b2-37} |
| 485 | Lₐ₁₋₈ | L_{b2-49} | 486 | Lₐ₁₋₈ | L_{b2-50} |
| 487 | Lₐ₁₋₈ | L_{b2-51} | 488 | Lₐ₁₋₈ | L_{b2-52} |
| 489 | Lₐ₁₋₈ | L_{b2-53} | 490 | Lₐ₁₋₈ | L_{b2-54} |
| 491 | Lₐ₁₋₈ | L_{b2-55} | 492 | Lₐ₁₋₈ | L_{b2-56} |
| 493 | Lₐ₁₋₈ | L_{b2-57} | 494 | Lₐ₁₋₈ | L_{b2-58} |
| 495 | Lₐ₁₋₈ | L_{b2-59} | 496 | Lₐ₁₋₈ | L_{b2-60} |
| 497 | Lₐ₁₋₈ | L_{b2-61} | 498 | Lₐ₁₋₈ | L_{b2-62} |
| 499 | Lₐ₁₋₈ | L_{b2-63} | 500 | Lₐ₁₋₈ | L_{b2-64} |
| 501 | Lₐ₁₋₈ | L_{b2-65} | 502 | Lₐ₁₋₈ | L_{b2-66} |
| 503 | Lₐ₁₋₈ | L_{b2-67} | 504 | Lₐ₁₋₈ | L_{b2-68} |
| 505 | Lₐ₁₋₈ | L_{b2-69} | 506 | Lₐ₁₋₈ | L_{b2-70} |
| 507 | Lₐ₁₋₈ | L_{b2-94} | 508 | Lₐ₁₋₈ | L_{b2-95} |
| 509 | Lₐ₁₋₈ | L_{b2-164} | 510 | Lₐ₁₋₈ | L_{b2-165} |
| 511 | Lₐ₁₋₈ | L_{b2-166} | 512 | Lₐ₁₋₈ | L_{b2-167} |
| 513 | Lₐ₁₋₈ | L_{b2-168} | 514 | Lₐ₁₋₈ | L_{b2-169} |
| 515 | Lₐ₁₋₈ | L_{b2-170} | 516 | Lₐ₁₋₈ | L_{b2-171} |
| 517 | Lₐ₁₋₈ | L_{b2-172} | 518 | Lₐ₁₋₈ | L_{b2-173} |
| 519 | Lₐ₁₋₈ | L_{b2-174} | 520 | Lₐ₁₋₈ | L_{b2-175} |
| 521 | Lₐ₁₋₈ | L_{b2-176} | 522 | Lₐ₁₋₈ | L_{b2-177} |
| 523 | Lₐ₁₋₈ | L_{b2-178} | 524 | Lₐ₁₋₈ | L_{b2-179} |
| 525 | Lₐ₁₋₈ | L_{b2-180} | 526 | Lₐ₁₋₈ | L_{b2-181} |
| 527 | Lₐ₁₋₈ | L_{b2-182} | 528 | Lₐ₁₋₈ | L_{b2-183} |
| 529 | Lₐ₁₋₈ | L_{b2-184} | 530 | Lₐ₁₋₈ | L_{b2-185} |
| 531 | Lₐ₁₋₈ | L_{b2-186} | 532 | Lₐ₁₋₈ | L_{b2-187} |
| 533 | Lₐ₁₋₈ | L_{b2-188} | 534 | Lₐ₁₋₈ | L_{b2-189} |
| 535 | Lₐ₁₋₈ | L_{b2-190} | 536 | Lₐ₁₋₈ | L_{b2-191} |
| 537 | Lₐ₁₋₈ | L_{b2-192} | 538 | Lₐ₁₋₈ | L_{b2-193} |
| 539 | Lₐ₁₋₈ | L_{b2-194} | 540 | Lₐ₁₋₈ | L_{b2-195} |
| 541 | Lₐ₁₋₈ | L_{b2-196} | 542 | Lₐ₁₋₈ | L_{b2-197} |
| 543 | Lₐ₁₋₈ | L_{b2-198} | 544 | Lₐ₁₋₈ | L_{b2-199} |
| 545 | Lₐ₁₋₈ | L_{b2-200} | 546 | Lₐ₁₋₈ | L_{b2-201} |
| 547 | Lₐ₁₋₈ | L_{b2-202} | 548 | Lₐ₁₋₈ | L_{b2-203} |
| 549 | Lₐ₁₋₈ | L_{b2-204} | 550 | Lₐ₁₋₈ | L_{b2-205} |
| 551 | Lₐ₁₋₈ | L_{b2-214} | 552 | Lₐ₁₋₈ | L_{b2-215} |
| 553 | Lₐ₁₋₈ | L_{b2-216} | 554 | Lₐ₁₋₈ | L_{b2-217} |
| 555 | Lₐ₁₋₈ | L_{b2-218} | 556 | Lₐ₁₋₈ | L_{b2-219} |
| 557 | Lₐ₁₋₈ | L_{b2-232} | 558 | Lₐ₁₋₈ | L_{b2-233} |
| 559 | Lₐ₁₋₈ | L_{b2-234} | 560 | Lₐ₁₋₈ | L_{b2-235} |
| 561 | Lₐ₁₋₈ | L_{b2-236} | 562 | Lₐ₁₋₈ | L_{b2-237} |
| 563 | Lₐ₁₋₈ | L_{b2-238} | 564 | Lₐ₁₋₈ | L_{b2-239} |
| 565 | Lₐ₁₋₈ | L_{b2-240} | 566 | Lₐ₁₋₈ | L_{b2-241} |
| 567 | Lₐ₁₋₈ | L_{b2-242} | 568 | Lₐ₁₋₈ | L_{b2-243} |
| 569 | Lₐ₁₋₈ | L_{b3-1} | 570 | Lₐ₁₋₈ | L_{b3-2} |
| 571 | Lₐ₁₋₈ | L_{b3-3} | 572 | Lₐ₁₋₈ | L_{b3-4} |
| 573 | Lₐ₁₋₈ | L_{b3-38} | 574 | Lₐ₁₋₈ | L_{b3-39} |
| 575 | Lₐ₁₋₈ | L_{b3-57} | 576 | Lₐ₁₋₈ | L_{b3-58} |
| 577 | Lₐ₁₋₈ | L_{b3-59} | 578 | Lₐ₁₋₈ | L_{b3-60} |
| 579 | Lₐ₁₋₈ | L_{b3-59} | 580 | Lₐ₁₋₈ | L_{b3-60} |
| 581 | Lₐ₁₋₈ | L_{b3-60} | 582 | Lₐ₁₋₈ | L_{b3-61} |
| 583 | Lₐ₁₋₈ | L_{b3-61} | 584 | Lₐ₁₋₈ | L_{b3-62} |
| 585 | Lₐ₁₋₈ | L_{b3-66} | 586 | Lₐ₁₋₈ | L_{b3-67} |
| 587 | Lₐ₁₋₈ | L_{b3-68} | 588 | Lₐ₁₋₈ | L_{b3-69} |
| 589 | Lₐ₁₋₈ | L_{b3-80} | 590 | Lₐ₁₋₈ | L_{b3-81} |
| 591 | Lₐ₁₋₈ | L_{b3-316} | 592 | Lₐ₁₋₈ | L_{b3-317} |
| 593 | Lₐ₁₋₈ | L_{b3-337} | 594 | Lₐ₁₋₈ | L_{b3-338} |
| 595 | Lₐ₁₋₈ | L_{b3-380} | 596 | Lₐ₁₋₈ | L_{b3-381} |
| 597 | Lₐ₁₋₈ | L_{b3-384} | 598 | Lₐ₁₋₈ | L_{b3-386} |
| 599 | Lₐ₁₋₈ | L_{b3-390} | 600 | Lₐ₁₋₈ | L_{b3-410} |
| 601 | Lₐ₁₋₈ | L_{b3-422} | 602 | Lₐ₁₋₈ | L_{b3-433} |
| 603 | Lₐ₁₋₈ | L_{b3-444} | 604 | Lₐ₁₋₈ | L_{b3-450} |
| 605 | Lₐ₁₋₈ | L_{b3-452} | 606 | Lₐ₁₋₈ | L_{b3-470} |
| 607 | Lₐ₁₋₈ | L_{b3-479} | 608 | Lₐ₁₋₈ | L_{b3-480} |
| 609 | Lₐ₁₋₈ | L_{b3-484} | 610 | Lₐ₁₋₈ | L_{b3-490} |
| 611 | Lₐ₁₋₈ | L_{b3-504} | 612 | Lₐ₁₋₈ | L_{b3-508} |
| 613 | Lₐ₁₋₈ | L_{b3-509} | 614 | Lₐ₁₋₈ | L_{b3-510} |
| 615 | Lₐ₁₋₈ | L_{b3-533} | 616 | Lₐ₁₋₈ | L_{b3-550} |
| 617 | Lₐ₁₋₈ | L_{b3-562} | 618 | Lₐ₁₋₈ | L_{b3-574} |
| 619 | Lₐ₁₋₈ | L_{b3-578} | 620 | Lₐ₁₋₈ | L_{b3-584} |
| 621 | Lₐ₁₋₁₃₇ | L_{b1-1} | 622 | Lₐ₁₋₁₃₇ | L_{b1-2} |
| 623 | Lₐ₁₋₁₃₇ | L_{b1-3} | 624 | Lₐ₁₋₁₃₇ | L_{b1-4} |
| 625 | Lₐ₁₋₁₃₇ | L_{b1-5} | 626 | Lₐ₁₋₁₃₇ | L_{b1-6} |
| 627 | Lₐ₁₋₁₃₇ | L_{b1-7} | 628 | Lₐ₁₋₁₃₇ | L_{b1-8} |
| 629 | Lₐ₁₋₁₃₇ | L_{b1-9} | 630 | Lₐ₁₋₁₃₇ | L_{b1-10} |
| 631 | Lₐ₁₋₁₃₇ | L_{b1-11} | 632 | Lₐ₁₋₁₃₇ | L_{b1-12} |
| 633 | Lₐ₁₋₁₃₇ | L_{b1-13} | 634 | Lₐ₁₋₁₃₇ | L_{b1-14} |
| 635 | Lₐ₁₋₁₃₇ | L_{b1-15} | 636 | Lₐ₁₋₁₃₇ | L_{b1-16} |
| 637 | Lₐ₁₋₁₃₇ | L_{b1-17} | 638 | Lₐ₁₋₁₃₇ | L_{b1-18} |
| 639 | Lₐ₁₋₁₃₇ | L_{b1-19} | 640 | Lₐ₁₋₁₃₇ | L_{b1-20} |
| 641 | Lₐ₁₋₁₃₇ | L_{b1-21} | 642 | Lₐ₁₋₁₃₇ | L_{b1-22} |
| 643 | Lₐ₁₋₁₃₇ | L_{b1-23} | 644 | Lₐ₁₋₁₃₇ | L_{b1-24} |
| 645 | Lₐ₁₋₁₃₇ | L_{b1-25} | 646 | Lₐ₁₋₁₃₇ | L_{b1-26} |
| 647 | Lₐ₁₋₁₃₇ | L_{b1-27} | 648 | Lₐ₁₋₁₃₇ | L_{b1-28} |
| 649 | Lₐ₁₋₁₃₇ | L_{b1-29} | 650 | Lₐ₁₋₁₃₇ | L_{b1-30} |
| 651 | Lₐ₁₋₁₃₇ | L_{b1-45} | 652 | Lₐ₁₋₁₃₇ | L_{b1-46} |
| 653 | Lₐ₁₋₁₃₇ | L_{b1-47} | 654 | Lₐ₁₋₁₃₇ | L_{b1-48} |
| 655 | Lₐ₁₋₁₃₇ | L_{b1-49} | 656 | Lₐ₁₋₁₃₇ | L_{b1-50} |
| 657 | Lₐ₁₋₁₃₇ | L_{b1-51} | 658 | Lₐ₁₋₁₃₇ | L_{b1-52} |
| 659 | Lₐ₁₋₁₃₇ | L_{b1-53} | 660 | Lₐ₁₋₁₃₇ | L_{b1-54} |
| 661 | Lₐ₁₋₁₃₇ | L_{b1-55} | 662 | Lₐ₁₋₁₃₇ | L_{b1-56} |
| 663 | Lₐ₁₋₁₃₇ | L_{b1-57} | 664 | Lₐ₁₋₁₃₇ | L_{b1-58} |
| 665 | Lₐ₁₋₁₃₇ | L_{b1-59} | 666 | Lₐ₁₋₁₃₇ | L_{b1-60} |
| 667 | Lₐ₁₋₁₃₇ | L_{b1-61} | 668 | Lₐ₁₋₁₃₇ | L_{b1-62} |
| 669 | Lₐ₁₋₁₃₇ | L_{b1-63} | 670 | Lₐ₁₋₁₃₇ | L_{b1-64} |
| 671 | Lₐ₁₋₁₃₇ | L_{b1-65} | 672 | Lₐ₁₋₁₃₇ | L_{b1-66} |
| 673 | Lₐ₁₋₁₃₇ | L_{b1-67} | 674 | Lₐ₁₋₁₃₇ | L_{b1-68} |
| 675 | Lₐ₁₋₁₃₇ | L_{b1-69} | 676 | Lₐ₁₋₁₃₇ | L_{b1-70} |
| 677 | Lₐ₁₋₁₃₇ | L_{b1-71} | 678 | Lₐ₁₋₁₃₇ | L_{b1-72} |
| 679 | Lₐ₁₋₁₃₇ | L_{b1-73} | 680 | Lₐ₁₋₁₃₇ | L_{b1-74} |
| 681 | Lₐ₁₋₁₃₇ | L_{b1-75} | 682 | Lₐ₁₋₁₃₇ | L_{b1-76} |
| 683 | Lₐ₁₋₁₃₇ | L_{b1-77} | 684 | Lₐ₁₋₁₃₇ | L_{b1-78} |
| 685 | Lₐ₁₋₁₃₇ | L_{b1-79} | 686 | Lₐ₁₋₁₃₇ | L_{b1-80} |
| 687 | Lₐ₁₋₁₃₇ | L_{b1-81} | 688 | Lₐ₁₋₁₃₇ | L_{b1-82} |
| 689 | Lₐ₁₋₁₃₇ | L_{b1-83} | 690 | Lₐ₁₋₁₃₇ | L_{b1-84} |
| 691 | Lₐ₁₋₁₃₇ | L_{b1-85} | 692 | Lₐ₁₋₁₃₇ | L_{b1-86} |
| 693 | Lₐ₁₋₁₃₇ | L_{b1-87} | 694 | Lₐ₁₋₁₃₇ | L_{b1-88} |
| 695 | Lₐ₁₋₁₃₇ | L_{b1-89} | 696 | Lₐ₁₋₁₃₇ | L_{b1-90} |
| 697 | Lₐ₁₋₁₃₇ | L_{b1-91} | 698 | Lₐ₁₋₁₃₇ | L_{b1-92} |
| 699 | Lₐ₁₋₁₃₇ | L_{b1-120} | 700 | Lₐ₁₋₁₃₇ | L_{b1-121} |
| 701 | Lₐ₁₋₁₃₇ | L_{b1-122} | 702 | Lₐ₁₋₁₃₇ | L_{b1-123} |
| 703 | Lₐ₁₋₁₃₇ | L_{b1-124} | 704 | Lₐ₁₋₁₃₇ | L_{b1-125} |
| 705 | Lₐ₁₋₁₃₇ | L_{b1-126} | 706 | Lₐ₁₋₁₃₇ | L_{b1-127} |
| 707 | Lₐ₁₋₁₃₇ | L_{b1-159} | 708 | Lₐ₁₋₁₃₇ | L_{b1-160} |
| 709 | Lₐ₁₋₁₃₇ | L_{b1-161} | 710 | Lₐ₁₋₁₃₇ | L_{b1-162} |
| 711 | Lₐ₁₋₁₃₇ | L_{b1-282} | 712 | Lₐ₁₋₁₃₇ | L_{b1-283} |
| 713 | Lₐ₁₋₁₃₇ | L_{b1-284} | 714 | Lₐ₁₋₁₃₇ | L_{b1-285} |
| 715 | Lₐ₁₋₁₃₇ | L_{b1-286} | 716 | Lₐ₁₋₁₃₇ | L_{b1-287} |
| 717 | Lₐ₁₋₁₃₇ | L_{b1-288} | 718 | Lₐ₁₋₁₃₇ | L_{b1-289} |
| 719 | Lₐ₁₋₁₃₇ | L_{b1-290} | 720 | Lₐ₁₋₁₃₇ | L_{b1-291} |
| 721 | Lₐ₁₋₁₃₇ | L_{b1-292} | 722 | Lₐ₁₋₁₃₇ | L_{b1-293} |
| 723 | Lₐ₁₋₁₃₇ | L_{b1-294} | 724 | Lₐ₁₋₁₃₇ | L_{b1-295} |
| 725 | Lₐ₁₋₁₃₇ | L_{b1-296} | 726 | Lₐ₁₋₁₃₇ | L_{b1-297} |
| 727 | Lₐ₁₋₁₃₇ | L_{b1-298} | 728 | Lₐ₁₋₁₃₇ | L_{b1-299} |
| 729 | Lₐ₁₋₁₃₇ | L_{b1-300} | 730 | Lₐ₁₋₁₃₇ | L_{b1-301} |
| 731 | Lₐ₁₋₁₃₇ | L_{b1-302} | 732 | Lₐ₁₋₁₃₇ | L_{b1-303} |
| 733 | Lₐ₁₋₁₃₇ | L_{b1-304} | 734 | Lₐ₁₋₁₃₇ | L_{b1-305} |
| 735 | Lₐ₁₋₁₃₇ | L_{b1-306} | 736 | Lₐ₁₋₁₃₇ | L_{b1-307} |
| 737 | Lₐ₁₋₁₃₇ | L_{b1-308} | 738 | Lₐ₁₋₁₃₇ | L_{b1-309} |
| 739 | Lₐ₁₋₁₃₇ | L_{b1-310} | 740 | Lₐ₁₋₁₃₇ | L_{b1-311} |
| 741 | Lₐ₁₋₁₃₇ | L_{b1-312} | 742 | Lₐ₁₋₁₃₇ | L_{b1-313} |
| 743 | Lₐ₁₋₁₃₇ | L_{b1-314} | 744 | Lₐ₁₋₁₃₇ | L_{b1-315} |
| 745 | Lₐ₁₋₁₃₇ | L_{b1-316} | 746 | Lₐ₁₋₁₃₇ | L_{b1-317} |
| 747 | Lₐ₁₋₁₃₇ | L_{b1-318} | 748 | Lₐ₁₋₁₃₇ | L_{b1-319} |
| 749 | Lₐ₁₋₁₃₇ | L_{b1-320} | 750 | Lₐ₁₋₁₃₇ | L_{b1-321} |
| 751 | Lₐ₁₋₁₃₇ | L_{b1-322} | 752 | Lₐ₁₋₁₃₇ | L_{b1-323} |
| 753 | Lₐ₁₋₁₃₇ | L_{b1-342} | 754 | Lₐ₁₋₁₃₇ | L_{b1-343} |
| 755 | Lₐ₁₋₁₃₇ | L_{b1-344} | 756 | Lₐ₁₋₁₃₇ | L_{b1-344} |
| 757 | Lₐ₁₋₁₃₇ | L_{b1-346} | 758 | Lₐ₁₋₁₃₇ | L_{b1-345} |
| 759 | Lₐ₁₋₁₃₇ | L_{b1-348} | 760 | Lₐ₁₋₁₃₇ | L_{b1-346} |
| 761 | Lₐ₁₋₁₃₇ | L_{b1-350} | 762 | Lₐ₁₋₁₃₇ | L_{b1-347} |
| 763 | Lₐ₁₋₁₃₇ | L_{b1-352} | 764 | Lₐ₁₋₁₃₇ | L_{b1-348} |
| 765 | Lₐ₁₋₁₃₇ | L_{b1-354} | 766 | Lₐ₁₋₁₃₇ | L_{b1-349} |
| 767 | Lₐ₁₋₁₃₇ | L_{b2-1} | 768 | Lₐ₁₋₁₃₇ | L_{b2-2} |
| 769 | Lₐ₁₋₁₃₇ | L_{b2-3} | 770 | Lₐ₁₋₁₃₇ | L_{b2-4} |
| 771 | Lₐ₁₋₁₃₇ | L_{b2-5} | 772 | Lₐ₁₋₁₃₇ | L_{b2-6} |
| 773 | Lₐ₁₋₁₃₇ | L_{b2-7} | 774 | Lₐ₁₋₁₃₇ | L_{b2-8} |
| 775 | Lₐ₁₋₁₃₇ | L_{b2-9} | 776 | Lₐ₁₋₁₃₇ | L_{b2-10} |
| 777 | Lₐ₁₋₁₃₇ | L_{b2-11} | 778 | Lₐ₁₋₁₃₇ | L_{b2-12} |
| 779 | Lₐ₁₋₁₃₇ | L_{b2-22} | 780 | Lₐ₁₋₁₃₇ | L_{b2-23} |
| 781 | Lₐ₁₋₁₃₇ | L_{b2-24} | 782 | Lₐ₁₋₁₃₇ | L_{b2-25} |
| 783 | Lₐ₁₋₁₃₇ | L_{b2-26} | 784 | Lₐ₁₋₁₃₇ | L_{b2-27} |
| 785 | Lₐ₁₋₁₃₇ | L_{b2-28} | 786 | Lₐ₁₋₁₃₇ | L_{b2-29} |
| 787 | Lₐ₁₋₁₃₇ | L_{b2-30} | 788 | Lₐ₁₋₁₃₇ | L_{b2-31} |
| 789 | Lₐ₁₋₁₃₇ | L_{b2-32} | 790 | Lₐ₁₋₁₃₇ | L_{b2-33} |
| 791 | Lₐ₁₋₁₃₇ | L_{b2-34} | 792 | Lₐ₁₋₁₃₇ | L_{b2-35} |
| 793 | Lₐ₁₋₁₃₇ | L_{b2-36} | 794 | Lₐ₁₋₁₃₇ | L_{b2-37} |
| 795 | Lₐ₁₋₁₃₇ | L_{b2-49} | 796 | Lₐ₁₋₁₃₇ | L_{b2-50} |
| 797 | Lₐ₁₋₁₃₇ | L_{b2-51} | 798 | Lₐ₁₋₁₃₇ | L_{b2-52} |
| 799 | Lₐ₁₋₁₃₇ | L_{b2-53} | 800 | Lₐ₁₋₁₃₇ | L_{b2-54} |
| 801 | Lₐ₁₋₁₃₇ | L_{b2-55} | 802 | Lₐ₁₋₁₃₇ | L_{b2-56} |
| 803 | Lₐ₁₋₁₃₇ | L_{b2-57} | 804 | Lₐ₁₋₁₃₇ | L_{b2-58} |
| 805 | Lₐ₁₋₁₃₇ | L_{b2-59} | 806 | Lₐ₁₋₁₃₇ | L_{b2-60} |
| 807 | Lₐ₁₋₁₃₇ | L_{b2-61} | 808 | Lₐ₁₋₁₃₇ | L_{b2-62} |
| 809 | Lₐ₁₋₁₃₇ | L_{b2-63} | 810 | Lₐ₁₋₁₃₇ | L_{b2-64} |
| 811 | Lₐ₁₋₁₃₇ | L_{b2-65} | 812 | Lₐ₁₋₁₃₇ | L_{b2-66} |
| 813 | Lₐ₁₋₁₃₇ | L_{b2-67} | 814 | Lₐ₁₋₁₃₇ | L_{b2-68} |
| 815 | Lₐ₁₋₁₃₇ | L_{b2-69} | 816 | Lₐ₁₋₁₃₇ | L_{b2-70} |
| 817 | Lₐ₁₋₁₃₇ | L_{b2-94} | 818 | Lₐ₁₋₁₃₇ | L_{b2-95} |
| 819 | Lₐ₁₋₁₃₇ | L_{b2-164} | 820 | Lₐ₁₋₁₃₇ | L_{b2-165} |
| 821 | Lₐ₁₋₁₃₇ | L_{b2-166} | 822 | Lₐ₁₋₁₃₇ | L_{b2-167} |
| 823 | Lₐ₁₋₁₃₇ | L_{b2-168} | 824 | Lₐ₁₋₁₃₇ | L_{b2-169} |
| 825 | Lₐ₁₋₁₃₇ | L_{b2-170} | 826 | Lₐ₁₋₁₃₇ | L_{b2-171} |
| 827 | Lₐ₁₋₁₃₇ | L_{b2-172} | 828 | Lₐ₁₋₁₃₇ | L_{b2-173} |
| 829 | Lₐ₁₋₁₃₇ | L_{b2-174} | 830 | Lₐ₁₋₁₃₇ | L_{b2-175} |
| 831 | Lₐ₁₋₁₃₇ | L_{b2-176} | 832 | Lₐ₁₋₁₃₇ | L_{b2-177} |
| 833 | Lₐ₁₋₁₃₇ | L_{b2-178} | 834 | Lₐ₁₋₁₃₇ | L_{b2-179} |
| 835 | Lₐ₁₋₁₃₇ | L_{b2-180} | 836 | Lₐ₁₋₁₃₇ | L_{b2-181} |
| 837 | Lₐ₁₋₁₃₇ | L_{b2-182} | 838 | Lₐ₁₋₁₃₇ | L_{b2-183} |
| 839 | Lₐ₁₋₁₃₇ | L_{b2-184} | 840 | Lₐ₁₋₁₃₇ | L_{b2-185} |
| 841 | Lₐ₁₋₁₃₇ | L_{b2-186} | 842 | Lₐ₁₋₁₃₇ | L_{b2-187} |
| 843 | Lₐ₁₋₁₃₇ | L_{b2-188} | 844 | Lₐ₁₋₁₃₇ | L_{b2-189} |
| 845 | Lₐ₁₋₁₃₇ | L_{b2-190} | 846 | Lₐ₁₋₁₃₇ | L_{b2-191} |
| 847 | Lₐ₁₋₁₃₇ | L_{b2-192} | 848 | Lₐ₁₋₁₃₇ | L_{b2-193} |
| 849 | Lₐ₁₋₁₃₇ | L_{b2-194} | 850 | Lₐ₁₋₁₃₇ | L_{b2-195} |
| 851 | Lₐ₁₋₁₃₇ | L_{b2-196} | 852 | Lₐ₁₋₁₃₇ | L_{b2-197} |
| 853 | Lₐ₁₋₁₃₇ | L_{b2-198} | 854 | Lₐ₁₋₁₃₇ | L_{b2-199} |
| 855 | Lₐ₁₋₁₃₇ | L_{b2-200} | 856 | Lₐ₁₋₁₃₇ | L_{b2-201} |
| 857 | Lₐ₁₋₁₃₇ | L_{b2-202} | 858 | Lₐ₁₋₁₃₇ | L_{b2-203} |
| 859 | Lₐ₁₋₁₃₇ | L_{b2-204} | 860 | Lₐ₁₋₁₃₇ | L_{b2-205} |
| 861 | Lₐ₁₋₁₃₇ | L_{b2-214} | 862 | Lₐ₁₋₁₃₇ | L_{b2-215} |
| 863 | Lₐ₁₋₁₃₇ | L_{b2-216} | 864 | Lₐ₁₋₁₃₇ | L_{b2-217} |
| 865 | Lₐ₁₋₁₃₇ | L_{b2-218} | 866 | Lₐ₁₋₁₃₇ | L_{b2-219} |
| 867 | Lₐ₁₋₁₃₇ | L_{b2-232} | 868 | Lₐ₁₋₁₃₇ | L_{b2-233} |
| 869 | Lₐ₁₋₁₃₇ | L_{b2-234} | 870 | Lₐ₁₋₁₃₇ | L_{b2-235} |
| 871 | Lₐ₁₋₁₃₇ | L_{b2-236} | 872 | Lₐ₁₋₁₃₇ | L_{b2-237} |
| 873 | Lₐ₁₋₁₃₇ | L_{b2-238} | 874 | Lₐ₁₋₁₃₇ | L_{b2-239} |
| 875 | Lₐ₁₋₁₃₇ | L_{b2-240} | 876 | Lₐ₁₋₁₃₇ | L_{b2-241} |
| 877 | Lₐ₁₋₁₃₇ | L_{b2-242} | 878 | Lₐ₁₋₁₃₇ | L_{b2-243} |
| 879 | Lₐ₁₋₁₃₇ | L_{b3-1} | 880 | Lₐ₁₋₁₃₇ | L_{b3-2} |
| 881 | Lₐ₁₋₁₃₇ | L_{b3-3} | 882 | Lₐ₁₋₁₃₇ | L_{b3-4} |
| 883 | Lₐ₁₋₁₃₇ | L_{b3-38} | 884 | Lₐ₁₋₁₃₇ | L_{b3-39} |
| 885 | Lₐ₁₋₁₃₇ | L_{b3-57} | 886 | Lₐ₁₋₁₃₇ | L_{b3-58} |
| 887 | Lₐ₁₋₁₃₇ | L_{b3-59} | 888 | Lₐ₁₋₁₃₇ | L_{b3-60} |
| 889 | Lₐ₁₋₁₃₇ | L_{b3-59} | 890 | Lₐ₁₋₁₃₇ | L_{b3-60} |
| 891 | Lₐ₁₋₁₃₇ | L_{b3-60} | 892 | Lₐ₁₋₁₃₇ | L_{b3-61} |
| 893 | Lₐ₁₋₁₃₇ | L_{b3-61} | 894 | Lₐ₁₋₁₃₇ | L_{b3-62} |
| 895 | Lₐ₁₋₁₃₇ | L_{b3-66} | 896 | Lₐ₁₋₁₃₇ | L_{b3-67} |
| 897 | Lₐ₁₋₁₃₇ | L_{b3-68} | 898 | Lₐ₁₋₁₃₇ | L_{b3-69} |
| 899 | Lₐ₁₋₁₃₇ | L_{b3-80} | 900 | Lₐ₁₋₁₃₇ | L_{b3-81} |
| 901 | Lₐ₁₋₁₃₇ | L_{b3-316} | 902 | Lₐ₁₋₁₃₇ | L_{b3-317} |
| 903 | Lₐ₁₋₁₃₇ | L_{b3-337} | 904 | Lₐ₁₋₁₃₇ | L_{b3-338} |
| 905 | Lₐ₁₋₁₃₇ | L_{b3-380} | 906 | Lₐ₁₋₁₃₇ | L_{b3-381} |
| 907 | Lₐ₁₋₁₃₇ | L_{b3-384} | 908 | Lₐ₁₋₁₃₇ | L_{b3-386} |
| 909 | Lₐ₁₋₁₃₇ | L_{b3-390} | 910 | Lₐ₁₋₁₃₇ | L_{b3-410} |
| 911 | Lₐ₁₋₁₃₇ | L_{b3-422} | 912 | Lₐ₁₋₁₃₇ | L_{b3-433} |
| 913 | Lₐ₁₋₁₃₇ | L_{b3-444} | 914 | Lₐ₁₋₁₃₇ | L_{b3-450} |
| 915 | Lₐ₁₋₁₃₇ | L_{b3-452} | 916 | Lₐ₁₋₁₃₇ | L_{b3-470} |
| 917 | Lₐ₁₋₁₃₇ | L_{b3-479} | 918 | Lₐ₁₋₁₃₇ | L_{b3-480} |
| 919 | Lₐ₁₋₁₃₇ | L_{b3-484} | 920 | Lₐ₁₋₁₃₇ | L_{b3-490} |
| 921 | Lₐ₁₋₁₃₇ | L_{b3-504} | 922 | Lₐ₁₋₁₃₇ | L_{b3-508} |
| 923 | Lₐ₁₋₁₃₇ | L_{b3-509} | 924 | Lₐ₁₋₁₃₇ | L_{b3-510} |
| 925 | Lₐ₁₋₁₃₇ | L_{b3-533} | 926 | Lₐ₁₋₁₃₇ | L_{b3-550} |
| 927 | Lₐ₁₋₁₃₇ | L_{b3-562} | 928 | Lₐ₁₋₁₃₇ | L_{b3-574} |
| 929 | Lₐ₁₋₁₃₇ | L_{b3-578} | 930 | Lₐ₁₋₁₃₇ | L_{b3-584} |
| 931 | Lₐ₂₋₁ | L_{b1-1} | 932 | Lₐ₂₋₁ | L_{b1-2} |
| 933 | Lₐ₂₋₁ | L_{b1-3} | 934 | Lₐ₂₋₁ | L_{b1-4} |
| 935 | Lₐ₂₋₁ | L_{b1-5} | 936 | Lₐ₂₋₁ | L_{b1-6} |
| 937 | Lₐ₂₋₁ | L_{b1-7} | 938 | Lₐ₂₋₁ | L_{b1-8} |
| 939 | Lₐ₂₋₁ | L_{b1-9} | 940 | Lₐ₂₋₁ | L_{b1-10} |
| 941 | Lₐ₂₋₁ | L_{b1-11} | 942 | Lₐ₂₋₁ | L_{b1-12} |
| 943 | Lₐ₂₋₁ | L_{b1-13} | 944 | Lₐ₂₋₁ | L_{b1-14} |
| 945 | Lₐ₂₋₁ | L_{b1-15} | 946 | Lₐ₂₋₁ | L_{b1-16} |
| 947 | Lₐ₂₋₁ | L_{b1-17} | 948 | Lₐ₂₋₁ | L_{b1-18} |
| 949 | Lₐ₂₋₁ | L_{b1-19} | 950 | Lₐ₂₋₁ | L_{b1-20} |
| 951 | Lₐ₂₋₁ | L_{b1-21} | 952 | Lₐ₂₋₁ | L_{b1-22} |
| 953 | Lₐ₂₋₁ | L_{b1-23} | 954 | Lₐ₂₋₁ | L_{b1-24} |
| 955 | Lₐ₂₋₁ | L_{b1-25} | 956 | Lₐ₂₋₁ | L_{b1-26} |
| 957 | Lₐ₂₋₁ | L_{b1-27} | 958 | Lₐ₂₋₁ | L_{b1-28} |
| 959 | Lₐ₂₋₁ | L_{b1-29} | 960 | Lₐ₂₋₁ | L_{b1-30} |
| 961 | Lₐ₂₋₁ | L_{b1-45} | 962 | Lₐ₂₋₁ | L_{b1-46} |
| 963 | Lₐ₂₋₁ | L_{b1-47} | 964 | Lₐ₂₋₁ | L_{b1-48} |
| 965 | Lₐ₂₋₁ | L_{b1-49} | 966 | Lₐ₂₋₁ | L_{b1-50} |
| 967 | Lₐ₂₋₁ | L_{b1-51} | 968 | Lₐ₂₋₁ | L_{b1-52} |
| 969 | Lₐ₂₋₁ | L_{b1-53} | 970 | Lₐ₂₋₁ | L_{b1-54} |
| 971 | Lₐ₂₋₁ | L_{b1-55} | 972 | Lₐ₂₋₁ | L_{b1-56} |
| 973 | Lₐ₂₋₁ | L_{b1-57} | 974 | Lₐ₂₋₁ | L_{b1-58} |
| 975 | Lₐ₂₋₁ | L_{b1-59} | 976 | Lₐ₂₋₁ | L_{b1-60} |
| 977 | Lₐ₂₋₁ | L_{b1-61} | 978 | Lₐ₂₋₁ | L_{b1-62} |
| 979 | Lₐ₂₋₁ | L_{b1-63} | 980 | Lₐ₂₋₁ | L_{b1-64} |
| 981 | Lₐ₂₋₁ | L_{b1-65} | 982 | Lₐ₂₋₁ | L_{b1-66} |
| 983 | Lₐ₂₋₁ | L_{b1-67} | 984 | Lₐ₂₋₁ | L_{b1-68} |
| 985 | Lₐ₂₋₁ | L_{b1-69} | 986 | Lₐ₂₋₁ | L_{b1-70} |
| 987 | Lₐ₂₋₁ | L_{b1-71} | 988 | Lₐ₂₋₁ | L_{b1-72} |
| 989 | Lₐ₂₋₁ | L_{b1-73} | 990 | Lₐ₂₋₁ | L_{b1-74} |
| 991 | Lₐ₂₋₁ | L_{b1-75} | 992 | Lₐ₂₋₁ | L_{b1-76} |
| 993 | Lₐ₂₋₁ | L_{b1-77} | 994 | Lₐ₂₋₁ | L_{b1-78} |
| 995 | Lₐ₂₋₁ | L_{b1-79} | 996 | Lₐ₂₋₁ | L_{b1-80} |
| 997 | Lₐ₂₋₁ | L_{b1-81} | 998 | Lₐ₂₋₁ | L_{b1-82} |
| 999 | Lₐ₂₋₁ | L_{b1-83} | 1000 | Lₐ₂₋₁ | L_{b1-84} |
| 1001 | Lₐ₂₋₁ | L_{b1-85} | 1002 | Lₐ₂₋₁ | L_{b1-86} |
| 1003 | Lₐ₂₋₁ | L_{b1-87} | 1004 | Lₐ₂₋₁ | L_{b1-88} |
| 1005 | Lₐ₂₋₁ | L_{b1-89} | 1006 | Lₐ₂₋₁ | L_{b1-90} |
| 1007 | Lₐ₂₋₁ | L_{b1-91} | 1008 | Lₐ₂₋₁ | L_{b1-92} |
| 1009 | Lₐ₂₋₁ | L_{b1-120} | 1010 | Lₐ₂₋₁ | L_{b1-121} |
| 1011 | Lₐ₂₋₁ | L_{b1-122} | 1012 | Lₐ₂₋₁ | L_{b1-123} |
| 1013 | Lₐ₂₋₁ | L_{b1-124} | 1014 | Lₐ₂₋₁ | L_{b1-125} |
| 1015 | Lₐ₂₋₁ | L_{b1-126} | 1016 | Lₐ₂₋₁ | L_{b1-127} |
| 1017 | Lₐ₂₋₁ | L_{b1-159} | 1018 | Lₐ₂₋₁ | L_{b1-160} |
| 1019 | Lₐ₂₋₁ | L_{b1-161} | 1020 | Lₐ₂₋₁ | L_{b1-162} |
| 1021 | Lₐ₂₋₁ | L_{b1-282} | 1022 | Lₐ₂₋₁ | L_{b1-283} |
| 1023 | Lₐ₂₋₁ | L_{b1-284} | 1024 | Lₐ₂₋₁ | L_{b1-285} |
| 1025 | Lₐ₂₋₁ | L_{b1-286} | 1026 | Lₐ₂₋₁ | L_{b1-287} |
| 1027 | Lₐ₂₋₁ | L_{b1-288} | 1028 | Lₐ₂₋₁ | L_{b1-289} |
| 1029 | Lₐ₂₋₁ | L_{b1-290} | 1030 | Lₐ₂₋₁ | L_{b1-291} |
| 1031 | Lₐ₂₋₁ | L_{b1-292} | 1032 | Lₐ₂₋₁ | L_{b1-293} |
| 1033 | Lₐ₂₋₁ | L_{b1-294} | 1034 | Lₐ₂₋₁ | L_{b1-295} |
| 1035 | Lₐ₂₋₁ | L_{b1-296} | 1036 | Lₐ₂₋₁ | L_{b1-297} |
| 1037 | Lₐ₂₋₁ | L_{b1-298} | 1038 | Lₐ₂₋₁ | L_{b1-299} |
| 1039 | Lₐ₂₋₁ | L_{b1-300} | 1040 | Lₐ₂₋₁ | L_{b1-301} |
| 1041 | Lₐ₂₋₁ | L_{b1-302} | 1042 | Lₐ₂₋₁ | L_{b1-303} |
| 1043 | Lₐ₂₋₁ | L_{b1-304} | 1044 | Lₐ₂₋₁ | L_{b1-305} |
| 1045 | Lₐ₂₋₁ | L_{b1-306} | 1046 | Lₐ₂₋₁ | L_{b1-307} |
| 1047 | Lₐ₂₋₁ | L_{b1-308} | 1048 | Lₐ₂₋₁ | L_{b1-309} |
| 1049 | Lₐ₂₋₁ | L_{b1-310} | 1050 | Lₐ₂₋₁ | L_{b1-311} |
| 1051 | Lₐ₂₋₁ | L_{b1-312} | 1052 | Lₐ₂₋₁ | L_{b1-313} |
| 1053 | Lₐ₂₋₁ | L_{b1-314} | 1054 | Lₐ₂₋₁ | L_{b1-315} |
| 1055 | Lₐ₂₋₁ | L_{b1-316} | 1056 | Lₐ₂₋₁ | L_{b1-317} |
| 1057 | Lₐ₂₋₁ | L_{b1-318} | 1058 | Lₐ₂₋₁ | L_{b1-319} |
| 1059 | Lₐ₂₋₁ | L_{b1-320} | 1060 | Lₐ₂₋₁ | L_{b1-321} |
| 1061 | Lₐ₂₋₁ | L_{b1-322} | 1062 | Lₐ₂₋₁ | L_{b1-323} |
| 1063 | Lₐ₂₋₁ | L_{b1-342} | 1064 | Lₐ₂₋₁ | L_{b1-343} |
| 1065 | Lₐ₂₋₁ | L_{b1-344} | 1066 | Lₐ₂₋₁ | L_{b1-344} |
| 1067 | Lₐ₂₋₁ | L_{b1-346} | 1068 | Lₐ₂₋₁ | L_{b1-345} |
| 1069 | Lₐ₂₋₁ | L_{b1-348} | 1070 | Lₐ₂₋₁ | L_{b1-346} |
| 1071 | Lₐ₂₋₁ | L_{b1-350} | 1072 | Lₐ₂₋₁ | L_{b1-347} |
| 1073 | Lₐ₂₋₁ | L_{b1-352} | 1074 | Lₐ₂₋₁ | L_{b1-348} |
| 1075 | Lₐ₂₋₁ | L_{b1-354} | 1076 | Lₐ₂₋₁ | L_{b1-349} |
| 1077 | Lₐ₂₋₁ | L_{b2-1} | 1078 | Lₐ₂₋₁ | L_{b2-2} |
| 1079 | Lₐ₂₋₁ | L_{b2-3} | 1080 | Lₐ₂₋₁ | L_{b2-4} |
| 1081 | Lₐ₂₋₁ | L_{b2-5} | 1082 | Lₐ₂₋₁ | L_{b2-6} |
| 1083 | Lₐ₂₋₁ | L_{b2-7} | 1084 | Lₐ₂₋₁ | L_{b2-8} |
| 1085 | Lₐ₂₋₁ | L_{b2-9} | 1086 | Lₐ₂₋₁ | L_{b2-10} |
| 1087 | Lₐ₂₋₁ | L_{b2-11} | 1088 | Lₐ₂₋₁ | L_{b2-12} |
| 1089 | Lₐ₂₋₁ | L_{b2-22} | 1090 | Lₐ₂₋₁ | L_{b2-23} |
| 1091 | Lₐ₂₋₁ | L_{b2-24} | 1092 | Lₐ₂₋₁ | L_{b2-25} |
| 1093 | Lₐ₂₋₁ | L_{b2-26} | 1094 | Lₐ₂₋₁ | L_{b2-27} |
| 1095 | Lₐ₂₋₁ | L_{b2-28} | 1096 | Lₐ₂₋₁ | L_{b2-29} |
| 1097 | Lₐ₂₋₁ | L_{b2-30} | 1098 | Lₐ₂₋₁ | L_{b2-31} |
| 1099 | Lₐ₂₋₁ | L_{b2-32} | 1100 | Lₐ₂₋₁ | L_{b2-33} |
| 1101 | Lₐ₂₋₁ | L_{b2-34} | 1102 | Lₐ₂₋₁ | L_{b2-35} |
| 1103 | Lₐ₂₋₁ | L_{b2-36} | 1104 | Lₐ₂₋₁ | L_{b2-37} |
| 1105 | Lₐ₂₋₁ | L_{b2-49} | 1106 | Lₐ₂₋₁ | L_{b2-50} |
| 1107 | Lₐ₂₋₁ | L_{b2-51} | 1108 | Lₐ₂₋₁ | L_{b2-52} |
| 1109 | Lₐ₂₋₁ | L_{b2-53} | 1110 | Lₐ₂₋₁ | L_{b2-54} |
| 1111 | Lₐ₂₋₁ | L_{b2-55} | 1112 | Lₐ₂₋₁ | L_{b2-56} |
| 1113 | Lₐ₂₋₁ | L_{b2-57} | 1114 | Lₐ₂₋₁ | L_{b2-58} |
| 1115 | Lₐ₂₋₁ | L_{b2-59} | 1116 | Lₐ₂₋₁ | L_{b2-60} |
| 1117 | Lₐ₂₋₁ | L_{b2-61} | 1118 | Lₐ₂₋₁ | L_{b2-62} |
| 1119 | Lₐ₂₋₁ | L_{b2-63} | 1120 | Lₐ₂₋₁ | L_{b2-64} |
| 1121 | Lₐ₂₋₁ | L_{b2-65} | 1122 | Lₐ₂₋₁ | L_{b2-66} |
| 1123 | Lₐ₂₋₁ | L_{b2-67} | 1124 | Lₐ₂₋₁ | L_{b2-68} |
| 1125 | Lₐ₂₋₁ | L_{b2-69} | 1126 | Lₐ₂₋₁ | L_{b2-70} |
| 1127 | Lₐ₂₋₁ | L_{b2-94} | 1128 | Lₐ₂₋₁ | L_{b2-95} |
| 1129 | Lₐ₂₋₁ | L_{b2-164} | 1130 | Lₐ₂₋₁ | L_{b2-165} |
| 1131 | Lₐ₂₋₁ | L_{b2-166} | 1132 | Lₐ₂₋₁ | L_{b2-167} |
| 1133 | Lₐ₂₋₁ | L_{b2-168} | 1134 | Lₐ₂₋₁ | L_{b2-169} |
| 1135 | Lₐ₂₋₁ | L_{b2-170} | 1136 | Lₐ₂₋₁ | L_{b2-171} |
| 1137 | Lₐ₂₋₁ | L_{b2-172} | 1138 | Lₐ₂₋₁ | L_{b2-173} |
| 1139 | Lₐ₂₋₁ | L_{b2-174} | 1140 | Lₐ₂₋₁ | L_{b2-175} |
| 1141 | Lₐ₂₋₁ | L_{b2-176} | 1142 | Lₐ₂₋₁ | L_{b2-177} |
| 1143 | Lₐ₂₋₁ | L_{b2-178} | 1144 | Lₐ₂₋₁ | L_{b2-179} |
| 1145 | Lₐ₂₋₁ | L_{b2-180} | 1146 | Lₐ₂₋₁ | L_{b2-181} |
| 1147 | Lₐ₂₋₁ | L_{b2-182} | 1148 | Lₐ₂₋₁ | L_{b2-183} |
| 1149 | Lₐ₂₋₁ | L_{b2-184} | 1150 | Lₐ₂₋₁ | L_{b2-185} |
| 1151 | Lₐ₂₋₁ | L_{b2-186} | 1152 | Lₐ₂₋₁ | L_{b2-187} |
| 1153 | Lₐ₂₋₁ | L_{b2-188} | 1154 | Lₐ₂₋₁ | L_{b2-189} |
| 1155 | Lₐ₂₋₁ | L_{b2-190} | 1156 | Lₐ₂₋₁ | L_{b2-191} |
| 1157 | Lₐ₂₋₁ | L_{b2-192} | 1158 | Lₐ₂₋₁ | L_{b2-193} |
| 1159 | Lₐ₂₋₁ | L_{b2-194} | 1160 | Lₐ₂₋₁ | L_{b2-195} |
| 1161 | Lₐ₂₋₁ | L_{b2-196} | 1162 | Lₐ₂₋₁ | L_{b2-197} |
| 1163 | Lₐ₂₋₁ | L_{b2-198} | 1164 | Lₐ₂₋₁ | L_{b2-199} |
| 1165 | Lₐ₂₋₁ | L_{b2-200} | 1166 | Lₐ₂₋₁ | L_{b2-201} |
| 1167 | Lₐ₂₋₁ | L_{b2-202} | 1168 | Lₐ₂₋₁ | L_{b2-203} |
| 1169 | Lₐ₂₋₁ | L_{b2-204} | 1170 | Lₐ₂₋₁ | L_{b2-205} |
| 1171 | Lₐ₂₋₁ | L_{b2-214} | 1172 | Lₐ₂₋₁ | L_{b2-215} |
| 1173 | Lₐ₂₋₁ | L_{b2-216} | 1174 | Lₐ₂₋₁ | L_{b2-217} |
| 1175 | Lₐ₂₋₁ | L_{b2-218} | 1176 | Lₐ₂₋₁ | L_{b2-219} |
| 1177 | Lₐ₂₋₁ | L_{b2-232} | 1178 | Lₐ₂₋₁ | L_{b2-233} |
| 1179 | Lₐ₂₋₁ | L_{b2-234} | 1180 | Lₐ₂₋₁ | L_{b2-235} |
| 1181 | Lₐ₂₋₁ | L_{b2-236} | 1182 | Lₐ₂₋₁ | L_{b2-237} |
| 1183 | Lₐ₂₋₁ | L_{b2-238} | 1184 | Lₐ₂₋₁ | L_{b2-239} |
| 1185 | Lₐ₂₋₁ | L_{b2-240} | 1186 | Lₐ₂₋₁ | L_{b2-241} |
| 1187 | Lₐ₂₋₁ | L_{b2-242} | 1188 | Lₐ₂₋₁ | L_{b2-243} |
| 1189 | Lₐ₂₋₁ | L_{b3-1} | 1190 | Lₐ₂₋₁ | L_{b3-2} |
| 1191 | Lₐ₂₋₁ | L_{b3-3} | 1192 | Lₐ₂₋₁ | L_{b3-4} |
| 1193 | Lₐ₂₋₁ | L_{b3-38} | 1194 | Lₐ₂₋₁ | L_{b3-39} |
| 1195 | Lₐ₂₋₁ | L_{b3-57} | 1196 | Lₐ₂₋₁ | L_{b3-58} |
| 1197 | Lₐ₂₋₁ | L_{b3-59} | 1198 | Lₐ₂₋₁ | L_{b3-60} |
| 1199 | Lₐ₂₋₁ | L_{b3-59} | 1200 | Lₐ₂₋₁ | L_{b3-60} |
| 1201 | Lₐ₂₋₁ | L_{b3-60} | 1202 | Lₐ₂₋₁ | L_{b3-61} |
| 1203 | Lₐ₂₋₁ | L_{b3-61} | 1204 | Lₐ₂₋₁ | L_{b3-62} |
| 1205 | Lₐ₂₋₁ | L_{b3-66} | 1206 | Lₐ₂₋₁ | L_{b3-67} |
| 1207 | Lₐ₂₋₁ | L_{b3-68} | 1208 | Lₐ₂₋₁ | L_{b3-69} |
| 1209 | Lₐ₂₋₁ | L_{b3-80} | 1210 | Lₐ₂₋₁ | L_{b3-81} |
| 1211 | Lₐ₂₋₁ | L_{b3-316} | 1212 | Lₐ₂₋₁ | L_{b3-317} |
| 1213 | Lₐ₂₋₁ | L_{b3-337} | 1214 | Lₐ₂₋₁ | L_{b3-338} |
| 1215 | Lₐ₂₋₁ | L_{b3-380} | 1216 | Lₐ₂₋₁ | L_{b3-381} |
| 1217 | Lₐ₂₋₁ | L_{b3-384} | 1218 | Lₐ₂₋₁ | L_{b3-386} |
| 1219 | Lₐ₂₋₁ | L_{b3-390} | 1220 | Lₐ₂₋₁ | L_{b3-410} |
| 1221 | Lₐ₂₋₁ | L_{b3-422} | 1222 | Lₐ₂₋₁ | L_{b3-433} |
| 1223 | Lₐ₂₋₁ | L_{b3-444} | 1224 | Lₐ₂₋₁ | L_{b3-450} |
| 1225 | Lₐ₂₋₁ | L_{b3-452} | 1226 | Lₐ₂₋₁ | L_{b3-470} |
| 1227 | Lₐ₂₋₁ | L_{b3-479} | 1228 | Lₐ₂₋₁ | L_{b3-480} |
| 1229 | Lₐ₂₋₁ | L_{b3-484} | 1230 | Lₐ₂₋₁ | L_{b3-490} |
| 1231 | Lₐ₂₋₁ | L_{b3-504} | 1232 | Lₐ₂₋₁ | L_{b3-508} |
| 1233 | Lₐ₂₋₁ | L_{b3-509} | 1234 | Lₐ₂₋₁ | L_{b3-510} |
| 1235 | Lₐ₂₋₁ | L_{b3-533} | 1236 | Lₐ₂₋₁ | L_{b3-550} |
| 1237 | Lₐ₂₋₁ | L_{b3-562} | 1238 | Lₐ₂₋₁ | L_{b3-574} |
| 1239 | Lₐ₂₋₁ | L_{b3-578} | 1240 | Lₐ₂₋₁ | L_{b3-584} |
| 1241 | Lₐ₁₋₂ | L_{b1-57} | 1242 | Lₐ₁₋₃ | L_{b1-57} |
| 1243 | Lₐ₁₋₄ | L_{b1-57} | 1244 | Lₐ₁₋₅ | L_{b1-57} |
| 1245 | Lₐ₁₋₆ | L_{b1-57} | 1246 | Lₐ₁₋₇ | L_{b1-57} |
| 1247 | Lₐ₁₋₉ | L_{b1-57} | 1248 | Lₐ₁₋₁₀ | L_{b1-57} |
| 1249 | Lₐ₁₋₁₁ | L_{b1-57} | 1250 | Lₐ₁₋₁₂ | L_{b1-57} |
| 1251 | Lₐ₁₋₁₃ | L_{b1-57} | 1252 | Lₐ₁₋₁₄ | L_{b1-57} |
| 1253 | Lₐ₁₋₁₅ | L_{b1-57} | 1254 | Lₐ₁₋₁₆ | L_{b1-57} |
| 1255 | Lₐ₁₋₁₇ | L_{b1-57} | 1256 | Lₐ₁₋₁₈ | L_{b1-57} |
| 1257 | Lₐ₁₋₁₉ | L_{b1-57} | 1258 | Lₐ₁₋₂₀ | L_{b1-57} |
| 1259 | Lₐ₁₋₂₁ | L_{b1-57} | 1260 | Lₐ₁₋₂₂ | L_{b1-57} |
| 1261 | Lₐ₁₋₂₃ | L_{b1-57} | 1262 | Lₐ₁₋₂₄ | L_{b1-57} |
| 1263 | Lₐ₁₋₂₅ | L_{b1-57} | 1264 | Lₐ₁₋₂₆ | L_{b1-57} |
| 1265 | Lₐ₁₋₂₇ | L_{b1-57} | 1266 | Lₐ₁₋₂₈ | L_{b1-57} |
| 1267 | Lₐ₁₋₂₉ | L_{b1-57} | 1268 | Lₐ₁₋₃₀ | L_{b1-57} |
| 1269 | Lₐ₁₋₃₁ | L_{b1-57} | 1270 | Lₐ₁₋₃₂ | L_{b1-57} |
| 1271 | Lₐ₁₋₃₃ | L_{b1-57} | 1272 | Lₐ₁₋₃₄ | L_{b1-57} |
| 1273 | Lₐ₁₋₃₅ | L_{b1-57} | 1274 | Lₐ₁₋₃₆ | L_{b1-57} |
| 1275 | Lₐ₁₋₃₇ | L_{b1-57} | 1276 | Lₐ₁₋₃₈ | L_{b1-57} |
| 1277 | Lₐ₁₋₃₉ | L_{b1-57} | 1278 | Lₐ₁₋₄₀ | L_{b1-57} |
| 1279 | Lₐ₁₋₄₁ | L_{b1-57} | 1280 | Lₐ₁₋₄₂ | L_{b1-57} |
| 1281 | Lₐ₁₋₄₃ | L_{b1-57} | 1282 | Lₐ₁₋₄₄ | L_{b1-57} |
| 1283 | Lₐ₁₋₄₅ | L_{b1-57} | 1284 | Lₐ₁₋₄₆ | L_{b1-57} |
| 1285 | Lₐ₁₋₄₇ | L_{b1-57} | 1286 | Lₐ₁₋₄₈ | L_{b1-57} |
| 1287 | Lₐ₁₋₄₉ | L_{b1-57} | 1288 | Lₐ₁₋₅₀ | L_{b1-57} |
| 1289 | Lₐ₁₋₅₁ | L_{b1-57} | 1290 | Lₐ₁₋₅₂ | L_{b1-57} |
| 1291 | Lₐ₁₋₉₀ | L_{b1-57} | 1292 | Lₐ₁₋₉₁ | L_{b1-57} |
| 1293 | Lₐ₁₋₉₂ | L_{b1-57} | 1294 | Lₐ₁₋₉₃ | L_{b1-57} |
| 1295 | Lₐ₁₋₉₄ | L_{b1-57} | 1296 | Lₐ₁₋₉₅ | L_{b1-57} |
| 1297 | Lₐ₁₋₉₆ | L_{b1-57} | 1298 | Lₐ₁₋₉₇ | L_{b1-57} |
| 1299 | Lₐ₁₋₉₈ | L_{b1-57} | 1300 | Lₐ₁₋₉₉ | L_{b1-57} |
| 1301 | Lₐ₁₋₁₀₀ | L_{b1-57} | 1302 | Lₐ₁₋₁₀₁ | L_{b1-57} |
| 1303 | Lₐ₁₋₁₀₂ | L_{b1-57} | 1304 | Lₐ₁₋₁₀₃ | L_{b1-57} |
| 1305 | Lₐ₁₋₁₀₄ | L_{b1-57} | 1306 | Lₐ₁₋₁₀₅ | L_{b1-57} |
| 1307 | Lₐ₁₋₁₀₆ | L_{b1-57} | 1308 | Lₐ₁₋₁₀₇ | L_{b1-57} |
| 1309 | Lₐ₁₋₁₀₈ | L_{b1-57} | 1310 | Lₐ₁₋₁₀₉ | L_{b1-57} |
| 1311 | Lₐ₁₋₁₁₀ | L_{b1-57} | 1312 | Lₐ₁₋₁₁₁ | L_{b1-57} |
| 1313 | Lₐ₁₋₁₁₂ | L_{b1-57} | 1314 | Lₐ₁₋₁₁₃ | L_{b1-57} |
| 1315 | Lₐ₁₋₁₁₄ | L_{b1-57} | 1316 | Lₐ₁₋₁₁₅ | L_{b1-57} |
| 1317 | Lₐ₁₋₁₁₆ | L_{b1-57} | 1318 | Lₐ₁₋₁₁₇ | L_{b1-57} |
| 1319 | Lₐ₁₋₁₃₉ | L_{b1-57} | 1320 | Lₐ₁₋₁₄₀ | L_{b1-57} |
| 1321 | Lₐ₁₋₁₄₁ | L_{b1-57} | 1322 | Lₐ₁₋₁₄₂ | L_{b1-57} |
| 1323 | Lₐ₁₋₁₄₃ | L_{b1-57} | 1324 | Lₐ₁₋₁₄₄ | L_{b1-57} |
| 1325 | Lₐ₁₋₁₄₅ | L_{b1-57} | 1326 | Lₐ₁₋₁₄₆ | L_{b1-57} |
| 1327 | Lₐ₁₋₁₄₇ | L_{b1-57} | 1328 | Lₐ₁₋₁₄₈ | L_{b1-57} |
| 1329 | Lₐ₁₋₁₄₉ | L_{b1-57} | 1330 | Lₐ₁₋₁₅₀ | L_{b1-57} |
| 1331 | Lₐ₁₋₁₅₁ | L_{b1-57} | 1332 | Lₐ₁₋₁₅₂ | L_{b1-57} |
| 1333 | Lₐ₁₋₁₅₃ | L_{b1-57} | 1334 | Lₐ₁₋₁₅₄ | L_{b1-57} |
| 1335 | Lₐ₁₋₁₅₅ | L_{b1-57} | 1336 | Lₐ₁₋₁₅₆ | L_{b1-57} |
| 1337 | Lₐ₁₋₁₅₇ | L_{b1-57} | 1338 | Lₐ₁₋₁₅₈ | L_{b1-57} |
| 1339 | Lₐ₁₋₁₅₉ | L_{b1-57} | 1340 | Lₐ₁₋₁₆₀ | L_{b1-57} |
| 1341 | Lₐ₁₋₁₆₁ | L_{b1-57} | 1342 | Lₐ₁₋₁₆₂ | L_{b1-57} |
| 1343 | Lₐ₁₋₁₆₃ | L_{b1-57} | 1344 | Lₐ₁₋₁₆₄ | L_{b1-57} |
| 1345 | Lₐ₁₋₁₆₅ | L_{b1-57} | 1346 | Lₐ₁₋₁₆₆ | L_{b1-57} |
| 1347 | Lₐ₁₋₁₆₇ | L_{b1-57} | 1348 | Lₐ₁₋₁₆₈ | L_{b1-57} |
| 1349 | Lₐ₁₋₁₆₉ | L_{b1-57} | 1350 | Lₐ₁₋₁₇₀ | L_{b1-57} |
| 1351 | Lₐ₁₋₁₇₁ | L_{b1-57} | 1352 | Lₐ₁₋₁₇₂ | L_{b1-57} |
| 1353 | Lₐ₁₋₁₇₃ | L_{b1-57} | 1354 | Lₐ₁₋₁₇₄ | L_{b1-57} |
| 1355 | Lₐ₁₋₁₇₅ | L_{b1-57} | 1356 | Lₐ₁₋₁₇₆ | L_{b1-57} |
| 1357 | Lₐ₁₋₁₇₇ | L_{b1-57} | 1358 | Lₐ₁₋₁₇₈ | L_{b1-57} |
| 1359 | Lₐ₁₋₁₇₉ | L_{b1-57} | 1360 | Lₐ₁₋₁₈₀ | L_{b1-57} |
| 1361 | Lₐ₁₋₁₈₁ | L_{b1-57} | 1362 | Lₐ₁₋₁₈₂ | L_{b1-57} |
| 1363 | Lₐ₁₋₁₈₃ | L_{b1-57} | 1364 | Lₐ₁₋₁₈₄ | L_{b1-57} |
| 1365 | Lₐ₁₋₁₈₅ | L_{b1-57} | 1366 | Lₐ₁₋₁₈₆ | L_{b1-57} |
| 1367 | Lₐ₂₋₂ | L_{b1-57} | 1368 | Lₐ₂₋₃ | L_{b1-57} |
| 1369 | Lₐ₂₋₄ | L_{b1-57} | 1370 | Lₐ₂₋₅ | L_{b1-57} |
| 1371 | Lₐ₂₋₆ | L_{b1-57} | 1372 | Lₐ₂₋₇ | L_{b1-57} |
| 1373 | Lₐ₂₋₈ | L_{b1-57} | 1374 | Lₐ₂₋₉ | L_{b1-57} |
| 1375 | Lₐ₂₋₁₆ | L_{b1-57} | 1376 | Lₐ₂₋₁₇ | L_{b1-57} |
| 1377 | Lₐ₂₋₁₈ | L_{b1-57} | 1378 | Lₐ₂₋₁₉ | L_{b1-57} |
| 1379 | Lₐ₂₋₂₀ | L_{b1-57} | 1380 | Lₐ₂₋₂₁ | L_{b1-57} |
| 1381 | Lₐ₂₋₂₂ | L_{b1-57} | 1382 | Lₐ₂₋₂₃ | L_{b1-57} |
| 1383 | Lₐ₂₋₃₁ | L_{b1-57} | 1384 | Lₐ₂₋₃₃ | L_{b1-57} |
| 1385 | Lₐ₂₋₄₆ | L_{b1-57} | 1386 | Lₐ₂₋₄₇ | L_{b1-57} |
| 1387 | Lₐ₂₋₄₈ | L_{b1-57} | 1388 | Lₐ₂₋₄₉ | L_{b1-57} |
| 1389 | Lₐ₂₋₅₀ | L_{b1-57} | 1390 | Lₐ₂₋₅₁ | L_{b1-57} |
| 1391 | Lₐ₂₋₅₂ | L_{b1-57} | 1392 | Lₐ₂₋₅₃ | L_{b1-57} |
| 1393 | Lₐ₂₋₅₄ | L_{b1-57} | 1394 | Lₐ₂₋₅₅ | L_{b1-57} |
| 1395 | Lₐ₂₋₅₆ | L_{b1-57} | 1396 | Lₐ₂₋₅₇ | L_{b1-57} |
| 1397 | Lₐ₂₋₅₈ | L_{b1-57} | 1398 | Lₐ₂₋₅₉ | L_{b1-57} |
| 1399 | Lₐ₂₋₆₀ | L_{b1-57} | 1400 | Lₐ₂₋₆₁ | L_{b1-57} |
| 1401 | Lₐ₂₋₆₂ | L_{b1-57} | 1402 | Lₐ₂₋₆₃ | L_{b1-57} |
| 1403 | Lₐ₂₋₆₄ | L_{b1-57} | 1404 | Lₐ₂₋₆₅ | L_{b1-57} |
| 1405 | Lₐ₂₋₆₆ | L_{b1-57} | 1406 | Lₐ₂₋₆₇ | L_{b1-57} |
| 1407 | Lₐ₂₋₁₄₉ | L_{b1-57} | 1408 | Lₐ₂₋₁₅₀ | L_{b1-57} |
| 1409 | Lₐ₂₋₁₅₁ | L_{b1-57} | 1410 | Lₐ₂₋₁₅₂ | L_{b1-57} |
| 1411 | Lₐ₂₋₁₅₃ | L_{b1-57} | 1412 | Lₐ₂₋₁₅₄ | L_{b1-57} |
| 1413 | Lₐ₂₋₁₅₅ | L_{b1-57} | 1414 | Lₐ₂₋₁₅₆ | L_{b1-57} |
| 1415 | Lₐ₂₋₁₅₇ | L_{b1-57} | 1416 | Lₐ₂₋₁₅₈ | L_{b1-57} |
| 1417 | Lₐ₂₋₁₅₉ | L_{b1-57} | 1418 | Lₐ₂₋₁₆₀ | L_{b1-57} |
| 1419 | Lₐ₃₋₁ | L_{b1-57} | 1420 | L₃₋₆ | L_{b1-57} |
| 1421 | Lₐ₃₋₇ | L_{b1-57} | 1422 | Lₐ₃₋₂₀ | L_{b1-57} |
| 1423 | Lₐ₃₋₂₁ | L_{b1-57} | 1424 | Lₐ₃₋₂₃ | L_{b1-57} |
| 1425 | Lₐ₃₋₂₂ | L_{b1-57} | 1426 | Lₐ₃₋₂₅ | L_{b1-57} |
| 1427 | Lₐ₃₋₄₁ | L_{b1-57} | 1428 | Lₐ₃₋₄₂ | L_{b1-57} |
| 1429 | Lₐ₃₋₄₃ | L_{b1-57} | 1430 | Lₐ₃₋₄₄ | L_{b1-57} |
| 1431 | Lₐ₃₋₅₄ | L_{b1-57} | 1432 | Lₐ₃₋₅₅ | L_{b1-57} |
| 1433 | Lₐ₃₋₇₃ | L_{b1-57} | 1434 | Lₐ₃₋₇₄ | L_{b1-57} |
| 1435 | Lₐ₃₋₇₅ | L_{b1-57} | 1436 | Lₐ₃₋₇₆ | L_{b1-57} |
| 1437 | Lₐ₃₋₇₇ | L_{b1-57} | 1438 | Lₐ₃₋₇₈ | L_{b1-57} |
| 1439 | Lₐ₃₋₇₉ | L_{b1-57} | 1440 | Lₐ₃₋₈₀ | L_{b1-57} |
| 1441 | Lₐ₃₋₈₁ | L_{b1-57} | 1442 | Lₐ₃₋₈₂ | L_{b1-57} |
| 1443 | Lₐ₃₋₈₃ | L_{b1-57} | 1444 | Lₐ₃₋₈₄ | L_{b1-57} |
| 1445 | Lₐ₃₋₈₅ | L_{b1-57} | 1446 | Lₐ₃₋₈₆ | L_{b1-57} |
| 1447 | Lₐ₃₋₈₇ | L_{b1-57} | 1448 | Lₐ₃₋₈₈ | L_{b1-57} |
| 1449 | Lₐ₃₋₉₉ | L_{b1-57} | 1450 | Lₐ₃₋₁₀₀ | L_{b1-57} |
| 1451 | Lₐ₃₋₁₀₁ | L_{b1-57} | 1452 | Lₐ₃₋₁₀₂ | L_{b1-57} |
| 1453 | Lₐ₃₋₁₀₃ | L_{b1-57} | 1454 | Lₐ₃₋₁₀₄ | L_{b1-57} |
| 1455 | Lₐ₃₋₁₀₅ | L_{b1-57} | 1456 | Lₐ₃₋₁₀₆ | L_{b1-57} |
| 1457 | Lₐ₃₋₁₀₇ | L_{b1-57} | 1458 | Lₐ₃₋₁₀₈ | L_{b1-57} |
| 1459 | Lₐ₃₋₁₀₉ | L_{b1-57} | 1460 | Lₐ₃₋₁₁₀ | L_{b1-57} |
| 1461 | Lₐ₃₋₁₁₁ | L_{b1-57} | 1462 | Lₐ₃₋₁₂₇ | L_{b1-57} |
| 1463 | Lₐ₁₋₂ | L_{b1-282} | 1464 | Lₐ₁₋₃ | L_{b1-282} |
| 1465 | Lₐ₁₋₄ | L_{b1-282} | 1466 | Lₐ₁₋₅ | L_{b1-282} |
| 1467 | Lₐ₁₋₆ | L_{b1-282} | 1468 | Lₐ₁₋₇ | L_{b1-282} |
| 1469 | Lₐ₁₋₉ | L_{b1-282} | 1470 | Lₐ₁₋₁₀ | L_{b1-282} |
| 1471 | Lₐ₁₋₁₁ | L_{b1-282} | 1472 | Lₐ₁₋₁₂ | L_{b1-282} |
| 1473 | Lₐ₁₋₁₃ | L_{b1-282} | 1474 | Lₐ₁₋₁₄ | L_{b1-282} |
| 1475 | Lₐ₁₋₁₅ | L_{b1-282} | 1476 | Lₐ₁₋₁₆ | L_{b1-282} |
| 1477 | Lₐ₁₋₁₇ | L_{b1-282} | 1478 | Lₐ₁₋₁₈ | L_{b1-282} |
| 1479 | Lₐ₁₋₁₉ | L_{b1-282} | 1480 | Lₐ₁₋₂₀ | L_{b1-282} |
| 1481 | Lₐ₁₋₂₁ | L_{b1-282} | 1482 | Lₐ₁₋₂₂ | L_{b1-282} |
| 1483 | Lₐ₁₋₂₃ | L_{b1-282} | 1484 | Lₐ₁₋₂₄ | L_{b1-282} |
| 1485 | Lₐ₁₋₂₅ | L_{b1-282} | 1486 | Lₐ₁₋₂₆ | L_{b1-282} |
| 1487 | Lₐ₁₋₂₇ | L_{b1-282} | 1488 | Lₐ₁₋₂₈ | L_{b1-282} |
| 1489 | Lₐ₁₋₂₉ | L_{b1-282} | 1490 | Lₐ₁₋₃₀ | L_{b1-282} |
| 1491 | Lₐ₁₋₃₁ | L_{b1-282} | 1492 | Lₐ₁₋₃₂ | L_{b1-282} |
| 1493 | Lₐ₁₋₃₃ | L_{b1-282} | 1494 | Lₐ₁₋₃₄ | L_{b1-282} |
| 1495 | Lₐ₁₋₃₅ | L_{b1-282} | 1496 | Lₐ₁₋₃₆ | L_{b1-282} |
| 1497 | Lₐ₁₋₃₇ | L_{b1-282} | 1498 | Lₐ₁₋₃₈ | L_{b1-282} |
| 1499 | Lₐ₁₋₃₉ | L_{b1-282} | 1500 | Lₐ₁₋₄₀ | L_{b1-282} |
| 1501 | Lₐ₁₋₄₁ | L_{b1-282} | 1502 | Lₐ₁₋₄₂ | L_{b1-282} |
| 1503 | Lₐ₁₋₄₃ | L_{b1-282} | 1504 | Lₐ₁₋₄₄ | L_{b1-282} |
| 1505 | Lₐ₁₋₄₅ | L_{b1-282} | 1506 | Lₐ₁₋₄₆ | L_{b1-282} |
| 1507 | Lₐ₁₋₄₇ | L_{b1-282} | 1508 | Lₐ₁₋₄₈ | L_{b1-282} |
| 1509 | Lₐ₁₋₄₉ | L_{b1-282} | 1510 | Lₐ₁₋₅₀ | L_{b1-282} |
| 1511 | Lₐ₁₋₅₁ | L_{b1-282} | 1512 | Lₐ₁₋₅₂ | L_{b1-282} |
| 1513 | Lₐ₁₋₉₀ | L_{b1-282} | 1514 | Lₐ₁₋₉₁ | L_{b1-282} |
| 1515 | Lₐ₁₋₉₂ | L_{b1-282} | 1516 | Lₐ₁₋₉₃ | L_{b1-282} |
| 1517 | Lₐ₁₋₉₄ | L_{b1-282} | 1518 | Lₐ₁₋₉₅ | L_{b1-282} |
| 1519 | Lₐ₁₋₉₆ | L_{b1-282} | 1520 | Lₐ₁₋₉₇ | L_{b1-282} |
| 1521 | Lₐ₁₋₉₈ | L_{b1-282} | 1522 | Lₐ₁₋₉₉ | L_{b1-282} |
| 1523 | Lₐ₁₋₁₀₀ | L_{b1-282} | 1524 | Lₐ₁₋₁₀₁ | L_{b1-282} |
| 1525 | Lₐ₁₋₁₀₂ | L_{b1-282} | 1526 | Lₐ₁₋₁₀₃ | L_{b1-282} |
| 1527 | Lₐ₁₋₁₀₄ | L_{b1-282} | 1528 | Lₐ₁₋₁₀₅ | L_{b1-282} |
| 1529 | Lₐ₁₋₁₀₆ | L_{b1-282} | 1530 | Lₐ₁₋₁₀₇ | L_{b1-282} |
| 1531 | Lₐ₁₋₁₀₈ | L_{b1-282} | 1532 | Lₐ₁₋₁₀₉ | L_{b1-282} |
| 1533 | Lₐ₁₋₁₁₀ | L_{b1-282} | 1534 | Lₐ₁₋₁₁₁ | L_{b1-282} |
| 1535 | Lₐ₁₋₁₁₂ | L_{b1-282} | 1536 | Lₐ₁₋₁₁₃ | L_{b1-282} |
| 1537 | Lₐ₁₋₁₁₄ | L_{b1-282} | 1538 | Lₐ₁₋₁₁₅ | L_{b1-282} |
| 1539 | Lₐ₁₋₁₁₆ | L_{b1-282} | 1540 | Lₐ₁₋₁₁₇ | L_{b1-282} |
| 1541 | Lₐ₁₋₁₃₉ | L_{b1-282} | 1542 | Lₐ₁₋₁₄₀ | L_{b1-282} |
| 1543 | Lₐ₁₋₁₄₁ | L_{b1-282} | 1544 | Lₐ₁₋₁₄₂ | L_{b1-282} |
| 1545 | Lₐ₁₋₁₄₃ | L_{b1-282} | 1546 | Lₐ₁₋₁₄₄ | L_{b1-282} |
| 1547 | Lₐ₁₋₁₄₅ | L_{b1-282} | 1548 | Lₐ₁₋₁₄₆ | L_{b1-282} |
| 1549 | Lₐ₁₋₁₄₇ | L_{b1-282} | 1550 | Lₐ₁₋₁₄₈ | L_{b1-282} |
| 1551 | Lₐ₁₋₁₄₉ | L_{b1-282} | 1552 | Lₐ₁₋₁₅₀ | L_{b1-282} |
| 1553 | Lₐ₁₋₁₅₁ | L_{b1-282} | 1554 | Lₐ₁₋₁₅₂ | L_{b1-282} |
| 1555 | Lₐ₁₋₁₅₃ | L_{b1-282} | 1556 | Lₐ₁₋₁₅₄ | L_{b1-282} |
| 1557 | Lₐ₁₋₁₅₅ | L_{b1-282} | 1558 | Lₐ₁₋₁₅₆ | L_{b1-282} |
| 1559 | Lₐ₁₋₁₅₇ | L_{b1-282} | 1560 | Lₐ₁₋₁₅₈ | L_{b1-282} |
| 1561 | Lₐ₁₋₁₅₉ | L_{b1-282} | 1562 | Lₐ₁₋₁₆₀ | L_{b1-282} |
| 1563 | Lₐ₁₋₁₆₁ | L_{b1-282} | 1564 | Lₐ₁₋₁₆₂ | L_{b1-282} |
| 1565 | Lₐ₁₋₁₆₃ | L_{b1-282} | 1566 | Lₐ₁₋₁₆₄ | L_{b1-282} |
| 1567 | Lₐ₁₋₁₆₅ | L_{b1-282} | 1568 | Lₐ₁₋₁₆₆ | L_{b1-282} |
| 1569 | Lₐ₁₋₁₆₇ | L_{b1-282} | 1570 | Lₐ₁₋₁₆₈ | L_{b1-282} |
| 1571 | Lₐ₁₋₁₆₉ | L_{b1-282} | 1572 | Lₐ₁₋₁₇₀ | L_{b1-282} |
| 1573 | Lₐ₁₋₁₇₁ | L_{b1-282} | 1574 | Lₐ₁₋₁₇₂ | L_{b1-282} |
| 1575 | Lₐ₁₋₁₇₃ | L_{b1-282} | 1576 | Lₐ₁₋₁₇₄ | L_{b1-282} |
| 1577 | Lₐ₁₋₁₇₅ | L_{b1-282} | 1578 | Lₐ₁₋₁₇₆ | L_{b1-282} |
| 1579 | Lₐ₁₋₁₇₇ | L_{b1-282} | 1580 | Lₐ₁₋₁₇₈ | L_{b1-282} |
| 1581 | Lₐ₁₋₁₇₉ | L_{b1-282} | 1582 | Lₐ₁₋₁₈₀ | L_{b1-282} |
| 1583 | Lₐ₁₋₁₈₁ | L_{b1-282} | 1584 | Lₐ₁₋₁₈₂ | L_{b1-282} |
| 1585 | Lₐ₁₋₁₈₃ | L_{b1-282} | 1586 | Lₐ₁₋₁₈₄ | L_{b1-282} |
| 1587 | Lₐ₁₋₁₈₅ | L_{b1-282} | 1588 | Lₐ₁₋₁₈₆ | L_{b1-282} |
| 1589 | Lₐ₂₋₂ | L_{b1-282} | 1590 | Lₐ₂₋₃ | L_{b1-282} |
| 1591 | Lₐ₂₋₄ | L_{b1-282} | 1592 | Lₐ₂₋₅ | L_{b1-282} |
| 1593 | Lₐ₂₋₆ | L_{b1-282} | 1594 | Lₐ₂₋₇ | L_{b1-282} |
| 1595 | Lₐ₂₋₈ | L_{b1-282} | 1596 | Lₐ₂₋₉ | L_{b1-282} |
| 1597 | Lₐ₂₋₁₆ | L_{b1-282} | 1598 | Lₐ₂₋₁₇ | L_{b1-282} |
| 1599 | Lₐ₂₋₁₈ | L_{b1-282} | 1600 | Lₐ₂₋₁₉ | L_{b1-282} |
| 1601 | Lₐ₂₋₂₀ | L_{b1-282} | 1602 | Lₐ₂₋₂₁ | L_{b1-282} |
| 1603 | Lₐ₂₋₂₂ | L_{b1-282} | 1604 | Lₐ₂₋₂₃ | L_{b1-282} |
| 1605 | Lₐ₂₋₃₁ | L_{b1-282} | 1606 | Lₐ₂₋₃₃ | L_{b1-282} |
| 1607 | Lₐ₂₋₄₆ | L_{b1-282} | 1608 | Lₐ₂₋₄₇ | L_{b1-282} |
| 1609 | Lₐ₂₋₄₈ | L_{b1-282} | 1610 | Lₐ₂₋₄₉ | L_{b1-282} |
| 1611 | Lₐ₂₋₅₀ | L_{b1-282} | 1612 | Lₐ₂₋₅₁ | L_{b1-282} |
| 1613 | Lₐ₂₋₅₂ | L_{b1-282} | 1614 | Lₐ₂₋₅₃ | L_{b1-282} |
| 1615 | Lₐ₂₋₅₄ | L_{b1-282} | 1616 | Lₐ₂₋₅₅ | L_{b1-282} |
| 1617 | Lₐ₂₋₅₆ | L_{b1-282} | 1618 | Lₐ₂₋₅₇ | L_{b1-282} |
| 1619 | Lₐ₂₋₅₈ | L_{b1-282} | 1620 | Lₐ₂₋₅₉ | L_{b1-282} |
| 1621 | Lₐ₂₋₆₀ | L_{b1-282} | 1622 | Lₐ₂₋₆₁ | L_{b1-282} |
| 1623 | Lₐ₂₋₆₂ | L_{b1-282} | 1624 | Lₐ₂₋₆₃ | L_{b1-282} |
| 1625 | Lₐ₂₋₆₄ | L_{b1-282} | 1626 | Lₐ₂₋₆₅ | L_{b1-282} |
| 1627 | Lₐ₂₋₆₆ | L_{b1-282} | 1628 | Lₐ₂₋₆₇ | L_{b1-282} |
| 1629 | Lₐ₂₋₁₄₉ | L_{b1-282} | 1630 | Lₐ₂₋₁₅₀ | L_{b1-282} |
| 1631 | Lₐ₂₋₁₅₁ | L_{b1-282} | 1632 | Lₐ₂₋₁₅₂ | L_{b1-282} |
| 1633 | Lₐ₂₋₁₅₃ | L_{b1-282} | 1634 | Lₐ₂₋₁₅₄ | L_{b1-282} |
| 1635 | Lₐ₂₋₁₅₅ | L_{b1-282} | 1636 | Lₐ₂₋₁₅₆ | L_{b1-282} |
| 1637 | Lₐ₂₋₁₅₇ | L_{b1-282} | 1638 | Lₐ₂₋₁₅₈ | L_{b1-282} |
| 1639 | Lₐ₂₋₁₅₉ | L_{b1-282} | 1640 | Lₐ₂₋₁₆₀ | L_{b1-282} |
| 1641 | Lₐ₃₋₁ | L_{b1-282} | 1642 | L₃₋₆ | L_{b1-282} |
| 1643 | Lₐ₃₋₇ | L_{b1-282} | 1644 | Lₐ₃₋₂₀ | L_{b1-282} |
| 1645 | Lₐ₃₋₂₁ | L_{b1-282} | 1646 | Lₐ₃₋₂₃ | L_{b1-282} |
| 1647 | Lₐ₃₋₂₂ | L_{b1-282} | 1648 | Lₐ₃₋₂₅ | L_{b1-282} |
| 1649 | Lₐ₃₋₄₁ | L_{b1-282} | 1650 | Lₐ₃₋₄₂ | L_{b1-282} |
| 1651 | Lₐ₃₋₄₃ | L_{b1-282} | 1652 | Lₐ₃₋₄₄ | L_{b1-282} |
| 1653 | Lₐ₃₋₅₄ | L_{b1-282} | 1654 | Lₐ₃₋₅₅ | L_{b1-282} |
| 1655 | Lₐ₃₋₇₃ | L_{b1-282} | 1656 | Lₐ₃₋₇₄ | L_{b1-282} |
| 1657 | Lₐ₃₋₇₅ | L_{b1-282} | 1658 | Lₐ₃₋₇₆ | L_{b1-282} |
| 1659 | Lₐ₃₋₇₇ | L_{b1-282} | 1660 | Lₐ₃₋₇₈ | L_{b1-282} |
| 1661 | Lₐ₃₋₇₉ | L_{b1-282} | 1662 | Lₐ₃₋₈₀ | L_{b1-282} |
| 1663 | Lₐ₃₋₈₁ | L_{b1-282} | 1664 | Lₐ₃₋₈₂ | L_{b1-282} |
| 1665 | Lₐ₃₋₈₃ | L_{b1-282} | 1666 | Lₐ₃₋₈₄ | L_{b1-282} |
| 1667 | Lₐ₃₋₈₅ | L_{b1-282} | 1668 | Lₐ₃₋₈₆ | L_{b1-282} |
| 1669 | Lₐ₃₋₈₇ | L_{b1-282} | 1670 | Lₐ₃₋₈₈ | L_{b1-282} |
| 1671 | Lₐ₃₋₉₉ | L_{b1-282} | 1672 | Lₐ₃₋₁₀₀ | L_{b1-282} |
| 1673 | Lₐ₃₋₁₀₁ | L_{b1-282} | 1674 | Lₐ₃₋₁₀₂ | L_{b1-282} |
| 1675 | Lₐ₃₋₁₀₃ | L_{b1-282} | 1676 | Lₐ₃₋₁₀₄ | L_{b1-282} |
| 1677 | Lₐ₃₋₁₀₅ | L_{b1-282} | 1678 | Lₐ₃₋₁₀₆ | L_{b1-282} |
| 1679 | Lₐ₃₋₁₀₇ | L_{b1-282} | 1680 | Lₐ₃₋₁₀₈ | L_{b1-282} |
| 1681 | Lₐ₃₋₁₀₉ | L_{b1-282} | 1682 | Lₐ₃₋₁₁₀ | L_{b1-282} |
| 1683 | Lₐ₃₋₁₁₁ | L_{b1-282} | 1684 | Lₐ₃₋₁₂₇ | L_{b1-282} |
| 1685 | Lₐ₁₋₂ | L_{b2-192} | 1686 | L_{b2-192} | L_{b2-192} |
| 1687 | Lₐ₁₋₄ | L_{b2-192} | 1688 | L_{b2-192} | L_{b2-192} |
| 1689 | Lₐ₁₋₆ | L_{b2-192} | 1690 | L_{b2-192} | L_{b2-192} |
| 1691 | Lₐ₁₋₉ | L_{b2-192} | 1692 | L_{b2-192} | L_{b2-192} |
| 1693 | Lₐ₁₋₁₁ | L_{b2-192} | 1694 | L_{b2-192} | L_{b2-192} |
| 1695 | Lₐ₁₋₁₃ | L_{b2-192} | 1696 | L_{b2-192} | L_{b2-192} |
| 1697 | Lₐ₁₋₁₅ | L_{b2-192} | 1698 | L_{b2-192} | L_{b2-192} |
| 1699 | Lₐ₁₋₁₇ | L_{b2-192} | 1700 | L_{b2-192} | L_{b2-192} |
| 1701 | Lₐ₁₋₁₉ | L_{b2-192} | 1702 | L_{b2-192} | L_{b2-192} |
| 1703 | Lₐ₁₋₂₁ | L_{b2-192} | 1704 | L_{b2-192} | L_{b2-192} |
| 1705 | Lₐ₁₋₂₃ | L_{b2-192} | 1706 | L_{b2-192} | L_{b2-192} |
| 1707 | Lₐ₁₋₂₅ | L_{b2-192} | 1708 | L_{b2-192} | L_{b2-192} |
| 1709 | Lₐ₁₋₂₇ | L_{b2-192} | 1710 | L_{b2-192} | L_{b2-192} |
| 1711 | Lₐ₁₋₂₉ | L_{b2-192} | 1712 | L_{b2-192} | L_{b2-192} |
| 1713 | Lₐ₁₋₃₁ | L_{b2-192} | 1714 | L_{b2-192} | L_{b2-192} |
| 1715 | Lₐ₁₋₃₃ | L_{b2-192} | 1716 | L_{b2-192} | L_{b2-192} |
| 1717 | Lₐ₁₋₃₅ | L_{b2-192} | 1718 | L_{b2-192} | L_{b2-192} |
| 1719 | Lₐ₁₋₃₇ | L_{b2-192} | 1720 | L_{b2-192} | L_{b2-192} |
| 1721 | Lₐ₁₋₃₉ | L_{b2-192} | 1722 | L_{b2-192} | L_{b2-192} |
| 1723 | Lₐ₁₋₄₁ | L_{b2-192} | 1724 | L_{b2-192} | L_{b2-192} |
| 1725 | Lₐ₁₋₄₃ | L_{b2-192} | 1726 | L_{b2-192} | L_{b2-192} |
| 1727 | Lₐ₁₋₄₅ | L_{b2-192} | 1728 | L_{b2-192} | L_{b2-192} |
| 1729 | Lₐ₁₋₄₇ | L_{b2-192} | 1730 | L_{b2-192} | L_{b2-192} |
| 1731 | Lₐ₁₋₄₉ | L_{b2-192} | 1732 | L_{b2-192} | L_{b2-192} |
| 1733 | Lₐ₁₋₅₁ | L_{b2-192} | 1734 | L_{b2-192} | L_{b2-192} |
| 1735 | Lₐ₁₋₉₀ | L_{b2-192} | 1736 | L_{b2-192} | L_{b2-192} |
| 1737 | Lₐ₁₋₉₂ | L_{b2-192} | 1738 | L_{b2-192} | L_{b2-192} |
| 1739 | Lₐ₁₋₉₄ | L_{b2-192} | 1740 | L_{b2-192} | L_{b2-192} |
| 1741 | Lₐ₁₋₉₆ | L_{b2-192} | 1742 | L_{b2-192} | L_{b2-192} |
| 1743 | Lₐ₁₋₉₈ | L_{b2-192} | 1744 | L_{b2-192} | L_{b2-192} |
| 1745 | Lₐ₁₋₁₀₀ | L_{b2-192} | 1746 | L_{b2-192} | L_{b2-192} |
| 1747 | Lₐ₁₋₁₀₂ | L_{b2-192} | 1748 | L_{b2-192} | L_{b2-192} |
| 1749 | Lₐ₁₋₁₀₄ | L_{b2-192} | 1750 | L_{b2-192} | L_{b2-192} |
| 1751 | Lₐ₁₋₁₀₆ | L_{b2-192} | 1752 | L_{b2-192} | L_{b2-192} |
| 1753 | Lₐ₁₋₁₀₈ | L_{b2-192} | 1754 | L_{b2-192} | L_{b2-192} |
| 1755 | Lₐ₁₋₁₁₀ | L_{b2-192} | 1756 | L_{b2-192} | L_{b2-192} |
| 1757 | Lₐ₁₋₁₁₂ | L_{b2-192} | 1758 | L_{b2-192} | L_{b2-192} |
| 1759 | Lₐ₁₋₁₁₄ | L_{b2-192} | 1760 | L_{b2-192} | L_{b2-192} |
| 1761 | Lₐ₁₋₁₁₆ | L_{b2-192} | 1762 | L_{b2-192} | L_{b2-192} |
| 1763 | Lₐ₁₋₁₃₉ | L_{b2-192} | 1764 | L_{b2-192} | L_{b2-192} |
| 1765 | Lₐ₁₋₁₄₁ | L_{b2-192} | 1766 | L_{b2-192} | L_{b2-192} |
| 1767 | Lₐ₁₋₁₄₃ | L_{b2-192} | 1768 | L_{b2-192} | L_{b2-192} |
| 1769 | Lₐ₁₋₁₄₅ | L_{b2-192} | 1770 | L_{b2-192} | L_{b2-192} |
| 1771 | Lₐ₁₋₁₄₇ | L_{b2-192} | 1772 | L_{b2-192} | L_{b2-192} |
| 1773 | Lₐ₁₋₁₄₉ | L_{b2-192} | 1774 | L_{b2-192} | L_{b2-192} |
| 1775 | Lₐ₁₋₁₅₁ | L_{b2-192} | 1776 | L_{b2-192} | L_{b2-192} |
| 1777 | Lₐ₁₋₁₅₃ | L_{b2-192} | 1778 | L_{b2-192} | L_{b2-192} |
| 1779 | Lₐ₁₋₁₅₅ | L_{b2-192} | 1780 | L_{b2-192} | L_{b2-192} |
| 1781 | Lₐ₁₋₁₅₇ | L_{b2-192} | 1782 | L_{b2-192} | L_{b2-192} |
| 1783 | Lₐ₁₋₁₅₉ | L_{b2-192} | 1784 | L_{b2-192} | L_{b2-192} |
| 1785 | Lₐ₁₋₁₆₁ | L_{b2-192} | 1786 | L_{b2-192} | L_{b2-192} |
| 1787 | Lₐ₁₋₁₆₃ | L_{b2-192} | 1788 | L_{b2-192} | L_{b2-192} |
| 1789 | Lₐ₁₋₁₆₅ | L_{b2-192} | 1790 | L_{b2-192} | L_{b2-192} |
| 1791 | Lₐ₁₋₁₆₇ | L_{b2-192} | 1792 | L_{b2-192} | L_{b2-192} |
| 1793 | Lₐ₁₋₁₆₉ | L_{b2-192} | 1794 | L_{b2-192} | L_{b2-192} |
| 1795 | Lₐ₁₋₁₇₁ | L_{b2-192} | 1796 | L_{b2-192} | L_{b2-192} |
| 1797 | Lₐ₁₋₁₇₃ | L_{b2-192} | 1798 | L_{b2-192} | L_{b2-192} |
| 1799 | Lₐ₁₋₁₇₅ | L_{b2-192} | 1800 | L_{b2-192} | L_{b2-192} |
| 1801 | Lₐ₁₋₁₇₇ | L_{b2-192} | 1802 | L_{b2-192} | L_{b2-192} |
| 1803 | Lₐ₁₋₁₇₉ | L_{b2-192} | 1804 | L_{b2-192} | L_{b2-192} |
| 1805 | Lₐ₁₋₁₈₁ | L_{b2-192} | 1806 | L_{b2-192} | L_{b2-192} |
| 1807 | Lₐ₁₋₁₈₃ | L_{b2-192} | 1808 | L_{b2-192} | L_{b2-192} |
| 1809 | Lₐ₁₋₁₈₅ | L_{b2-192} | 1810 | L_{b2-192} | L_{b2-192} |
| 1811 | Lₐ₂₋₂ | L_{b2-192} | 1812 | L_{b2-192} | L_{b2-192} |
| 1813 | Lₐ₂₋₄ | L_{b2-192} | 1814 | L_{b2-192} | L_{b2-192} |
| 1815 | Lₐ₂₋₆ | L_{b2-192} | 1816 | L_{b2-192} | L_{b2-192} |
| 1817 | Lₐ₂₋₈ | L_{b2-192} | 1818 | L_{b2-192} | L_{b2-192} |
| 1819 | Lₐ₂₋₁₆ | L_{b2-192} | 1820 | L_{b2-192} | L_{b2-192} |
| 1821 | Lₐ₂₋₁₈ | L_{b2-192} | 1822 | L_{b2-192} | L_{b2-192} |
| 1823 | Lₐ₂₋₂₀ | L_{b2-192} | 1824 | L_{b2-192} | L_{b2-192} |
| 1825 | Lₐ₂₋₂₂ | L_{b2-192} | 1826 | L_{b2-192} | L_{b2-192} |
| 1827 | Lₐ₂₋₃₁ | L_{b2-192} | 1828 | L_{b2-192} | L_{b2-192} |
| 1829 | Lₐ₂₋₄₆ | L_{b2-192} | 1830 | L_{b2-192} | L_{b2-192} |
| 1831 | Lₐ₂₋₄₈ | L_{b2-192} | 1832 | L_{b2-192} | L_{b2-192} |
| 1833 | Lₐ₂₋₅₀ | L_{b2-192} | 1834 | L_{b2-192} | L_{b2-192} |
| 1835 | Lₐ₂₋₅₂ | L_{b2-192} | 1836 | L_{b2-192} | L_{b2-192} |
| 1837 | Lₐ₂₋₅₄ | L_{b2-192} | 1838 | L_{b2-192} | L_{b2-192} |
| 1839 | Lₐ₂₋₅₆ | L_{b2-192} | 1840 | L_{b2-192} | L_{b2-192} |
| 1841 | Lₐ₂₋₅₈ | L_{b2-192} | 1842 | L_{b2-192} | L_{b2-192} |
| 1843 | Lₐ₂₋₆₀ | L_{b2-192} | 1844 | L_{b2-192} | L_{b2-192} |
| 1845 | Lₐ₂₋₆₂ | L_{b2-192} | 1846 | L_{b2-192} | L_{b2-192} |
| 1847 | Lₐ₂₋₆₄ | L_{b2-192} | 1848 | L_{b2-192} | L_{b2-192} |
| 1849 | Lₐ₂₋₆₆ | L_{b2-192} | 1850 | L_{b2-192} | L_{b2-192} |
| 1851 | Lₐ₂₋₁₄₉ | L_{b2-192} | 1852 | L_{b2-192} | L_{b2-192} |
| 1853 | Lₐ₂₋₁₅₁ | L_{b2-192} | 1854 | L_{b2-192} | L_{b2-192} |
| 1855 | Lₐ₂₋₁₅₃ | L_{b2-192} | 1856 | L_{b2-192} | L_{b2-192} |
| 1857 | Lₐ₂₋₁₅₅ | L_{b2-192} | 1858 | L_{b2-192} | L_{b2-192} |
| 1859 | Lₐ₂₋₁₅₇ | L_{b2-192} | 1860 | L_{b2-192} | L_{b2-192} |
| 1861 | Lₐ₂₋₁₅₉ | L_{b2-192} | 1862 | L_{b2-192} | L_{b2-192} |
| 1863 | Lₐ₃₋₁ | L_{b2-192} | 1864 | L_{b2-192} | L_{b2-192} |
| 1865 | Lₐ₃₋₇ | L_{b2-192} | 1866 | L_{b2-192} | L_{b2-192} |
| 1867 | Lₐ₃₋₂₁ | L_{b2-192} | 1868 | L_{b2-192} | L_{b2-192} |
| 1869 | Lₐ₃₋₂₂ | L_{b2-192} | 1870 | L_{b2-192} | L_{b2-192} |
| 1871 | Lₐ₃₋₄₁ | L_{b2-192} | 1872 | L_{b2-192} | L_{b2-192} |
| 1873 | Lₐ₃₋₄₃ | L_{b2-192} | 1874 | L_{b2-192} | L_{b2-192} |
| 1875 | Lₐ₃₋₅₄ | L_{b2-192} | 1876 | L_{b2-192} | L_{b2-192} |
| 1877 | Lₐ₃₋₇₃ | L_{b2-192} | 1878 | L_{b2-192} | L_{b2-192} |
| 1879 | Lₐ₃₋₇₅ | L_{b2-192} | 1880 | L_{b2-192} | L_{b2-192} |
| 1881 | Lₐ₃₋₇₇ | L_{b2-192} | 1882 | L_{b2-192} | L_{b2-192} |
| 1883 | Lₐ₃₋₇₉ | L_{b2-192} | 1884 | L_{b2-192} | L_{b2-192} |
| 1885 | Lₐ₃₋₈₁ | L_{b2-192} | 1886 | L_{b2-192} | L_{b2-192} |
| 1887 | Lₐ₃₋₈₃ | L_{b2-192} | 1888 | L_{b2-192} | L_{b2-192} |
| 1889 | Lₐ₃₋₈₅ | L_{b2-192} | 1890 | L_{b2-192} | L_{b2-192} |
| 1891 | Lₐ₃₋₈₇ | L_{b2-192} | 1892 | L_{b2-192} | L_{b2-192} |
| 1893 | Lₐ₃₋₉₉ | L_{b2-192} | 1894 | L_{b2-192} | L_{b2-192} |
| 1895 | Lₐ₃₋₁₀₁ | L_{b2-192} | 1896 | L_{b2-192} | L_{b2-192} |
| 1897 | Lₐ₃₋₁₀₃ | L_{b2-192} | 1898 | L_{b2-192} | L_{b2-192} |
| 1899 | Lₐ₃₋₁₀₅ | L_{b2-192} | 1900 | L_{b2-192} | L_{b2-192} |
| 1901 | Lₐ₃₋₁₀₇ | L_{b2-192} | 1902 | L_{b2-192} | L_{b2-192} |
| 1903 | Lₐ₃₋₁₀₉ | L_{b2-192} | 1904 | L_{b2-192} | L_{b2-192} |
| 1905 | Lₐ₃₋₁₁₁ | L_{b2-192} | 1906 | L_{b2-192} | L_{b2-192} |
| 1907 | Lₐ₁₋₂ | L_{b3-66} | 1908 | Lₐ₁₋₃ | L_{b3-66} |
| 1909 | Lₐ₁₋₄ | L_{b3-66} | 1910 | Lₐ₁₋₅ | L_{b3-66} |
| 1911 | Lₐ₁₋₆ | L_{b3-66} | 1912 | Lₐ₁₋₇ | L_{b3-66} |
| 1913 | Lₐ₁₋₉ | L_{b3-66} | 1914 | Lₐ₁₋₁₀ | L_{b3-66} |
| 1915 | Lₐ₁₋₁₁ | L_{b3-66} | 1916 | Lₐ₁₋₁₂ | L_{b3-66} |
| 1917 | Lₐ₁₋₁₃ | L_{b3-66} | 1918 | Lₐ₁₋₁₄ | L_{b3-66} |
| 1919 | Lₐ₁₋₁₅ | L_{b3-66} | 1920 | Lₐ₁₋₁₆ | L_{b3-66} |
| 1921 | Lₐ₁₋₁₇ | L_{b3-66} | 1922 | Lₐ₁₋₁₈ | L_{b3-66} |
| 1923 | Lₐ₁₋₁₉ | L_{b3-66} | 1924 | Lₐ₁₋₂₀ | L_{b3-66} |
| 1925 | Lₐ₁₋₂₁ | L_{b3-66} | 1926 | Lₐ₁₋₂₂ | L_{b3-66} |
| 1927 | Lₐ₁₋₂₃ | L_{b3-66} | 1928 | Lₐ₁₋₂₄ | L_{b3-66} |
| 1929 | Lₐ₁₋₂₅ | L_{b3-66} | 1930 | Lₐ₁₋₂₆ | L_{b3-66} |
| 1931 | Lₐ₁₋₂₇ | L_{b3-66} | 1932 | Lₐ₁₋₂₈ | L_{b3-66} |
| 1933 | Lₐ₁₋₂₉ | L_{b3-66} | 1934 | Lₐ₁₋₃₀ | L_{b3-66} |
| 1935 | Lₐ₁₋₃₁ | L_{b3-66} | 1936 | Lₐ₁₋₃₂ | L_{b3-66} |
| 1937 | Lₐ₁₋₃₃ | L_{b3-66} | 1938 | Lₐ₁₋₃₄ | L_{b3-66} |
| 1939 | Lₐ₁₋₃₅ | L_{b3-66} | 1940 | Lₐ₁₋₃₆ | L_{b3-66} |
| 1941 | Lₐ₁₋₃₇ | L_{b3-66} | 1942 | Lₐ₁₋₃₈ | L_{b3-66} |
| 1943 | Lₐ₁₋₃₉ | L_{b3-66} | 1944 | Lₐ₁₋₄₀ | L_{b3-66} |
| 1945 | Lₐ₁₋₄₁ | L_{b3-66} | 1946 | Lₐ₁₋₄₂ | L_{b3-66} |
| 1947 | Lₐ₁₋₄₃ | L_{b3-66} | 1948 | Lₐ₁₋₄₄ | L_{b3-66} |
| 1949 | Lₐ₁₋₄₅ | L_{b3-66} | 1950 | Lₐ₁₋₄₆ | L_{b3-66} |
| 1951 | Lₐ₁₋₄₇ | L_{b3-66} | 1952 | Lₐ₁₋₄₈ | L_{b3-66} |
| 1953 | Lₐ₁₋₄₉ | L_{b3-66} | 1954 | Lₐ₁₋₅₀ | L_{b3-66} |
| 1955 | Lₐ₁₋₅₁ | L_{b3-66} | 1956 | Lₐ₁₋₅₂ | L_{b3-66} |
| 1957 | Lₐ₁₋₉₀ | L_{b3-66} | 1958 | Lₐ₁₋₉₁ | L_{b3-66} |
| 1959 | Lₐ₁₋₉₂ | L_{b3-66} | 1960 | Lₐ₁₋₉₃ | L_{b3-66} |
| 1961 | Lₐ₁₋₉₄ | L_{b3-66} | 1962 | Lₐ₁₋₉₅ | L_{b3-66} |
| 1963 | Lₐ₁₋₉₆ | L_{b3-66} | 1964 | Lₐ₁₋₉₇ | L_{b3-66} |
| 1965 | Lₐ₁₋₉₈ | L_{b3-66} | 1966 | Lₐ₁₋₉₉ | L_{b3-66} |
| 1967 | Lₐ₁₋₁₀₀ | L_{b3-66} | 1968 | Lₐ₁₋₁₀₁ | L_{b3-66} |
| 1969 | Lₐ₁₋₁₀₂ | L_{b3-66} | 1970 | Lₐ₁₋₁₀₃ | L_{b3-66} |
| 1971 | Lₐ₁₋₁₀₄ | L_{b3-66} | 1972 | Lₐ₁₋₁₀₅ | L_{b3-66} |
| 1973 | Lₐ₁₋₁₀₆ | L_{b3-66} | 1974 | Lₐ₁₋₁₀₇ | L_{b3-66} |
| 1975 | Lₐ₁₋₁₀₈ | L_{b3-66} | 1976 | Lₐ₁₋₁₀₉ | L_{b3-66} |
| 1977 | Lₐ₁₋₁₁₀ | L_{b3-66} | 1978 | Lₐ₁₋₁₁₁ | L_{b3-66} |
| 1979 | Lₐ₁₋₁₁₂ | L_{b3-66} | 1980 | Lₐ₁₋₁₁₃ | L_{b3-66} |
| 1981 | Lₐ₁₋₁₁₄ | L_{b3-66} | 1982 | Lₐ₁₋₁₁₅ | L_{b3-66} |
| 1983 | Lₐ₁₋₁₁₆ | L_{b3-66} | 1984 | Lₐ₁₋₁₁₇ | L_{b3-66} |
| 1985 | Lₐ₁₋₁₃₉ | L_{b3-66} | 1986 | Lₐ₁₋₁₄₀ | L_{b3-66} |
| 1987 | Lₐ₁₋₁₄₁ | L_{b3-66} | 1988 | Lₐ₁₋₁₄₂ | L_{b3-66} |
| 1989 | Lₐ₁₋₁₄₃ | L_{b3-66} | 1990 | Lₐ₁₋₁₄₄ | L_{b3-66} |
| 1991 | Lₐ₁₋₁₄₅ | L_{b3-66} | 1992 | Lₐ₁₋₁₄₆ | L_{b3-66} |
| 1993 | Lₐ₁₋₁₄₇ | L_{b3-66} | 1994 | Lₐ₁₋₁₄₈ | L_{b3-66} |
| 1995 | Lₐ₁₋₁₄₉ | L_{b3-66} | 1996 | Lₐ₁₋₁₅₀ | L_{b3-66} |
| 1997 | Lₐ₁₋₁₅₁ | L_{b3-66} | 1998 | Lₐ₁₋₁₅₂ | L_{b3-66} |
| 1999 | Lₐ₁₋₁₅₃ | L_{b3-66} | 2000 | Lₐ₁₋₁₅₄ | L_{b3-66} |
| 2001 | Lₐ₁₋₁₅₅ | L_{b3-66} | 2002 | Lₐ₁₋₁₅₆ | L_{b3-66} |
| 2003 | Lₐ₁₋₁₅₇ | L_{b3-66} | 2004 | Lₐ₁₋₁₅₈ | L_{b3-66} |
| 2005 | Lₐ₁₋₁₅₉ | L_{b3-66} | 2006 | Lₐ₁₋₁₆₀ | L_{b3-66} |
| 2007 | Lₐ₁₋₁₆₁ | L_{b3-66} | 2008 | Lₐ₁₋₁₆₂ | L_{b3-66} |
| 2009 | Lₐ₁₋₁₆₃ | L_{b3-66} | 2010 | Lₐ₁₋₁₆₄ | L_{b3-66} |
| 2011 | Lₐ₁₋₁₆₅ | L_{b3-66} | 2012 | Lₐ₁₋₁₆₆ | L_{b3-66} |
| 2013 | Lₐ₁₋₁₆₇ | L_{b3-66} | 2014 | Lₐ₁₋₁₆₈ | L_{b3-66} |
| 2015 | Lₐ₁₋₁₆₉ | L_{b3-66} | 2016 | Lₐ₁₋₁₇₀ | L_{b3-66} |
| 2017 | Lₐ₁₋₁₇₁ | L_{b3-66} | 2018 | Lₐ₁₋₁₇₂ | L_{b3-66} |
| 2019 | Lₐ₁₋₁₇₃ | L_{b3-66} | 2020 | Lₐ₁₋₁₇₄ | L_{b3-66} |
| 2021 | Lₐ₁₋₁₇₅ | L_{b3-66} | 2022 | Lₐ₁₋₁₇₆ | L_{b3-66} |
| 2023 | Lₐ₁₋₁₇₇ | L_{b3-66} | 2024 | Lₐ₁₋₁₇₈ | L_{b3-66} |
| 2025 | Lₐ₁₋₁₇₉ | L_{b3-66} | 2026 | Lₐ₁₋₁₈₀ | L_{b3-66} |
| 2027 | Lₐ₁₋₁₈₁ | L_{b3-66} | 2028 | Lₐ₁₋₁₈₂ | L_{b3-66} |
| 2029 | Lₐ₁₋₁₈₃ | L_{b3-66} | 2030 | Lₐ₁₋₁₈₄ | L_{b3-66} |
| 2031 | Lₐ₁₋₁₈₅ | L_{b3-66} | 2032 | Lₐ₁₋₁₈₆ | L_{b3-66} |
| 2033 | Lₐ₂₋₂ | L_{b3-66} | 2034 | Lₐ₂₋₃ | L_{b3-66} |
| 2035 | Lₐ₂₋₄ | L_{b3-66} | 2036 | Lₐ₂₋₅ | L_{b3-66} |
| 2037 | Lₐ₂₋₆ | L_{b3-66} | 2038 | Lₐ₂₋₇ | L_{b3-66} |
| 2039 | Lₐ₂₋₈ | L_{b3-66} | 2040 | Lₐ₂₋₉ | L_{b3-66} |
| 2041 | Lₐ₂₋₁₆ | L_{b3-66} | 2042 | Lₐ₂₋₁₇ | L_{b3-66} |
| 2043 | Lₐ₂₋₁₈ | L_{b3-66} | 2044 | Lₐ₂₋₁₉ | L_{b3-66} |
| 2045 | Lₐ₂₋₂₀ | L_{b3-66} | 2046 | Lₐ₂₋₂₁ | L_{b3-66} |
| 2047 | Lₐ₂₋₂₂ | L_{b3-66} | 2048 | Lₐ₂₋₂₃ | L_{b3-66} |
| 2049 | Lₐ₂₋₃₁ | L_{b3-66} | 2050 | Lₐ₂₋₃₃ | L_{b3-66} |
| 2051 | Lₐ₂₋₄₆ | L_{b3-66} | 2052 | Lₐ₂₋₄₇ | L_{b3-66} |
| 2053 | Lₐ₂₋₄₈ | L_{b3-66} | 2054 | Lₐ₂₋₄₉ | L_{b3-66} |
| 2055 | Lₐ₂₋₅₀ | L_{b3-66} | 2056 | Lₐ₂₋₅₁ | L_{b3-66} |
| 2057 | Lₐ₂₋₅₂ | L_{b3-66} | 2058 | Lₐ₂₋₅₃ | L_{b3-66} |
| 2059 | Lₐ₂₋₅₄ | L_{b3-66} | 2060 | Lₐ₂₋₅₅ | L_{b3-66} |
| 2061 | Lₐ₂₋₅₆ | L_{b3-66} | 2062 | Lₐ₂₋₅₇ | L_{b3-66} |
| 2063 | Lₐ₂₋₅₈ | L_{b3-66} | 2064 | Lₐ₂₋₅₉ | L_{b3-66} |
| 2065 | Lₐ₂₋₆₀ | L_{b3-66} | 2066 | Lₐ₂₋₆₁ | L_{b3-66} |
| 2067 | Lₐ₂₋₆₂ | L_{b3-66} | 2068 | Lₐ₂₋₆₃ | L_{b3-66} |
| 2069 | Lₐ₂₋₆₄ | L_{b3-66} | 2070 | Lₐ₂₋₆₅ | L_{b3-66} |
| 2071 | Lₐ₂₋₆₆ | L_{b3-66} | 2072 | Lₐ₂₋₆₇ | L_{b3-66} |
| 2073 | Lₐ₂₋₁₄₉ | L_{b3-66} | 2074 | Lₐ₂₋₁₅₀ | L_{b3-66} |
| 2075 | Lₐ₂₋₁₅₁ | L_{b3-66} | 2076 | Lₐ₂₋₁₅₂ | L_{b3-66} |
| 2077 | Lₐ₂₋₁₅₃ | L_{b3-66} | 2078 | Lₐ₂₋₁₅₄ | L_{b3-66} |
| 2079 | Lₐ₂₋₁₅₅ | L_{b3-66} | 2080 | Lₐ₂₋₁₅₆ | L_{b3-66} |
| 2081 | Lₐ₂₋₁₅₇ | L_{b3-66} | 2082 | Lₐ₂₋₁₅₈ | L_{b3-66} |
| 2083 | Lₐ₂₋₁₅₉ | L_{b3-66} | 2084 | Lₐ₂₋₁₆₀ | L_{b3-66} |
| 2085 | Lₐ₃₋₁ | L_{b3-66} | 2086 | L₃₋₆ | L_{b3-66} |
| 2087 | Lₐ₃₋₇ | L_{b3-66} | 2088 | Lₐ₃₋₂₀ | L_{b3-66} |
| 2089 | Lₐ₃₋₂₁ | L_{b3-66} | 2090 | Lₐ₃₋₂₃ | L_{b3-66} |
| 2091 | Lₐ₃₋₂₂ | L_{b3-66} | 2092 | Lₐ₃₋₂₅ | L_{b3-66} |
| 2093 | Lₐ₃₋₄₁ | L_{b3-66} | 2094 | Lₐ₃₋₄₂ | L_{b3-66} |
| 2095 | Lₐ₃₋₄₃ | L_{b3-66} | 2096 | Lₐ₃₋₄₄ | L_{b3-66} |
| 2097 | Lₐ₃₋₅₄ | L_{b3-66} | 2098 | Lₐ₃₋₅₅ | L_{b3-66} |
| 2099 | Lₐ₃₋₇₃ | L_{b3-66} | 2100 | Lₐ₃₋₇₄ | L_{b3-66} |
| 2101 | Lₐ₃₋₇₅ | L_{b3-66} | 2102 | Lₐ₃₋₇₆ | L_{b3-66} |
| 2103 | Lₐ₃₋₇₇ | L_{b3-66} | 2104 | Lₐ₃₋₇₈ | L_{b3-66} |
| 2105 | Lₐ₃₋₇₉ | L_{b3-66} | 2106 | Lₐ₃₋₈₀ | L_{b3-66} |
| 2107 | Lₐ₃₋₈₁ | L_{b3-66} | 2108 | Lₐ₃₋₈₂ | L_{b3-66} |
| 2109 | Lₐ₃₋₈₃ | L_{b3-66} | 2110 | Lₐ₃₋₈₄ | L_{b3-66} |
| 2111 | Lₐ₃₋₈₅ | L_{b3-66} | 2112 | Lₐ₃₋₈₆ | L_{b3-66} |
| 2113 | Lₐ₃₋₈₇ | L_{b3-66} | 2114 | Lₐ₃₋₈₈ | L_{b3-66} |
| 2115 | Lₐ₃₋₉₉ | L_{b3-66} | 2116 | Lₐ₃₋₁₀₀ | L_{b3-66} |
| 2117 | Lₐ₃₋₁₀₁ | L_{b3-66} | 2118 | Lₐ₃₋₁₀₂ | L_{b3-66} |
| 2119 | Lₐ₃₋₁₀₃ | L_{bS-66} | 2120 | Lₐ₃₋₁₀₄ | L_{b3-66} |
| 2121 | Lₐ₃₋₁₀₅ | L_{b3-66} | 2122 | Lₐ₃₋₁₀₆ | L_{b3-66} |
| 2123 | Lₐ₃₋₁₀₇ | L_{b3-66} | 2124 | Lₐ₃₋₁₀₈ | L_{b3-66} |
| 2125 | Lₐ₃₋₁₀₉ | L_{b3-66} | 2126 | Lₐ₃₋₁₁₀ | L_{b3-66} |
| 2127 | Lₐ₃₋₁₁₁ | L_{b3-66} | 2128 | Lₐ₃₋₁₂₇ | L_{b3-66} |

21. An electroluminescent device, comprising:
an anode,
a cathode, and
an organic layer disposed between the anode and the cathode, wherein at least one layer of the organic layer comprises the metal complex according to claim 1.

22. The electroluminescent device according to claim 21, wherein the organic layer comprising the metal complex is an emissive layer.

23. The electroluminescent device according to claim 22, wherein the emissive layer further comprises a first host compound;
preferably, the emissive layer further comprises a second host compound; and
more preferably, the first host compound and/or the second host compound comprises at least one chemical group selected from the group consisting of: benzene, pyridine, pyrimidine, triazine, carbazole, azacarbazole, indolocarbazole, dibenzothiophene, azadibenzothiophene, dibenzofuran, azadibenzofuran, dibenzoselenophene, triphenylene, azatriphenylene, fluorene, silafluorene, naphthalene, quinoline, isoquinoline, quinazoline, quinoxaline, phenanthrene, azaphenanthrene and combinations thereof.

24. The electroluminescent device according to claim 23, wherein the metal complex is doped in the first host compound and the second host compound, and the weight of the metal complex accounts for 1% to 30% of the total weight of the emissive layer; and
preferably, the weight of the metal complex accounts for 3% to 13% of the total weight of the emissive layer.

25. A compound composition, comprising the metal complex according to claim 1.
